SEQUENCE LISTING (1065 sequences)

SEQ ID NO: 1 - protein, 232 aa, Homo sapiens
NSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIEHKIPVDADFLYAYSTAPGYYSWRNSKDGSWFIQSLCAMLKQYADKLEFMHILTRVNRKVATEFESFSFDATFHAKKQIPCIVSMLTKELYFYH

SEQ ID NO: 2 - protein, 183 aa, Homo sapiens
GDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKNLLVCSVNGFYPGSIEVRWFRNGQEEKTGVVSTGLIQNGDWTFQTLVMLETVPRSGEVYTCQVEHPSLTSPLTVEWRA

SEQ ID NO: 3 - protein, 302 aa, Homo sapiens
SATYDKGYQFEEENPLRDHPQPFEEGLRRLQEGDLPNAVLLFEAAVQQDPKHMEAWQYLGTTQAENEQELLAISALRRCLELKPDNQTALMALAVSFTNESLQRQACEILRDWLRYTPAYAHLVTRILGSLLSDSLFLEVKELFLAAVRLDPTSIDPDVQCGLGVLFNLSGEYDKAVDCFTAALSVRPNDYLLWNKLGATLANGNQSEEAVAAYRRALELQPGYIRSRYNLGISCINLGAHREAVEHFLEALNMQRKSGGAMSENIWSTLRLALSMLGQSDAYGAADARDLSTLLTMFGLPQ

SEQ ID NO: 4 - protein, 298 aa, Homo sapiens
MEMEKEFEQIDKSGSWAAIYQDIRHEASDFPCRVAKLPKNKNRNRYRDVSPFDHSRIKLHQEDNDYINASLIKMEEAQRSYILTQGPLPNTCGHFWEMVWEQKSRGVVMLNRVMEKGSLKCAQYWPQKEEKEMIFEDTNLKLTLISEDIKSYYTVRQLELENLTTQETREILHFHYTTWPDFGVPESPASFLNFLFKVRESGSLSPEHGPVVVHASAGIGRSGTFCLADTCLLLMDKRKDPSSVDIKKVLLEMRKFRMGLIQTADQLRFSYLAVIEGAKFIMGDSSVQDQWKELSHED

SEQ ID NO: 5 - protein, 107 aa, Homo sapiens
MDSIQAEEWYFGKITRRESERLLLNAENPRGTFLVRESETTKGAYCLSVSDFDNAKGLNVKHYKIRKLDSGGFYITSRTQFNSLQQLVAYYSKHADGLCHRLTTVCP

SEQ ID NO: 6 - protein, 426 aa, Homo sapiens
  VARIANT 426: note = Any amino acid
NPFQFYLTRVSGVKPKYNSGALHIKDILSPLFGTLVSSAQFNYCFDVDWLVKQYPPEFRKKPILLVHGDKREAKAHLHAQAKPYENISLCQAKLDIAFGTHHTKMMLLLYEEGLRVVIHTSNLIHADWHQKTQGIWLSPLYPRIADGTHKSGESPTHFKANLISYLTAYNAPSLKEWIDVIHKHDLSETNVYLIGSTPGRFQGSQKDNWGHFRLKKLLKDHASSMSWPVVGQFSSVGSLGADESKWLCSEFKESMLTLGVPLYLIYPSVENVRTSLEGYPAGGSLPYSIQTAEKQNWLHSYFHKWSAETSGRSNAMPHIKTYMRPSPDFSKIAWFLVTSANLSKAAWGALEKNGTQLMIRSYELGVLFLPSALGLDSFKVKQKFATFPVPYDLPPELYGSKDRPWIWNIPYVKAPDTHGNMWVPSX

SEQ ID NO: 7 - protein, 265 aa, Homo sapiens
  VARIANT 265: note = Any amino acid
VPFVEDWDLVQTLGEGAYGEVQLAVNRVTEEAVAVKIVDMKNIKKEICINKMLNHENVVKFYGHRREGNIQYLFLEYCSGGELFDRIEPDIGMPEPDAQRFFHQLMAGVVYLHGIGITHRDIKPENLLLDERDNLKISDFGLATVFRYNNRERLLNKMCGTLPYVAPELLKRREFHAEPVDVWSCGIVLTAMLAGELPWDQPSDSCQEYSDWKEKKTYLNPWKKIDSAPLALLHKILVENPSARITIPDIKKDRWYNKPLKKGAX

SEQ ID NO: 8 - protein, 112 aa, Homo sapiens
GSPIPHHDEKTWNVGSSNRNKAENLLRGKRDGTFLVRESSKQGCYACSVVVDGEVKHCVINKTATGYGFAEPYNLYSSLKELVLHYQHTSLVQHNDSLNVTLAYPVYAQQRR

SEQ ID NO: 9 - protein, 166 aa, Homo sapiens
  VARIANT 166: note = Any amino acid
VCPNIIKRSAWEARETHCPKMNLPAKYVIIIHTAGTSCTVSTDCQTVVRNIQSFHMDTRNFCDIGYHFLVGQDGGVYEGVGWHIQGSHTYGFNDIALGIAFIGYFVEKPPNAAALEAAQDLIQCAVVEGYLTPNYLLMGHSDVVNILSPGQALYNIISTWPHFKHX

SEQ ID NO: 10 - protein, 322 aa, Homo sapiens
KEPLINYLDMEYFGTISIGSPPQNFTVIFDTGSSNLWVPSVYCTSPACKTHSRFQPSQSSTYSQPGQSFSIQYGTGSLSGIIGADQVSVEGLTVVGQQFGESVTEPGQTFVDAEFDGILGLGYPSLAVGGVTPVFDNMMAQNLVDLPMFSVYMSSNPGAGSELIFGGYDHSHFSGSLNWVPVTKQAYWQIALDNIQVGGTVMFCSEGCQAIVDTGTSLITGPSDKIKQLQNAIGAAPVDGEYAVECANLNVMPDVTFTINGVPYTLSPTAYTLLDQFCSSGFQGLDIHPPAGPLWILGDVFIRQFYSVFDRGNNRVGLAPAV

SEQ ID NO: 11 - protein, 164 aa, Homo sapiens
VNPTVFFDIAVDGEPLGRVSFELFADKVPKTAENFRALSTGEKGFGYKGSCFHRIIPGFMCQGGDFTRHNGTGGKSIYGEKFEDENFILKHTGPGILSMANAGPNTNGSQFFICTAKTEWLDGKHVVFGKVKEGMNIVEAMERFGSRNGKTSKKITIADCGQLE

SEQ ID NO: 12 - protein, 164 aa, Homo sapiens; identical to SEQ ID NO: 11

SEQ ID NO: 13 - protein, 272 aa, Homo sapiens
  VARIANT 272: note = Any amino acid
VPFVEDWDLVQTLGEGAYGEVQLAVNRVTEEAVAVKIVDMKRAVDCPENIKKEICINAMLNHENVVKFYGHRREGNIQYLFLEYCSGGELFDRIEPDIGMPEPDAQRFFHQLMAGVVYLHGIGITHRDIKPENLLLDERDNLKISDFGLATVFRYNNRERLLNKMCGTLPYVAPELLKRREFHAEPVDVWSCGIVLTAMLAGELPWDQPSDSCQEYSDWKEKKTYLNPWKKIDSAPLALLHKILVENPSARITIPDIKKDRWYNKPLKKGAX

SEQ ID NO: 14 - protein, 304 aa, Homo sapiens
PVKPNYALKFTLAGHTKAVSSVKFSPNGEWLASSSADKLIKIWGAYDGKFEKTISGHKLGISDVAWSSDSNLLVSASDDKTLKIWDVSSGKCLKTLKGHSNYVFCCNFNPQSNLIVSGSFDESVRIWDVKTGKCLKTLPAHSDPVSAVHFNRDGSLIVSSSYDGLCRIWDTASGQCLKTLIDDDNPPVSFVKFSPNGKYILAATLDNTLKLWDYSKGKCLKTYTGHKNEKYCIFANFSVTGGKWIVSGSEDNLVYIWNLQTKEIVQKLQGHTDVVISTACHPTENIIASAALENDKTIKLWKSD

SEQ ID NO: 15 - protein, 303 aa, Homo sapiens
KPNYALKFTLAGHTKAVSSVKFSPNGEWLASSSADKLIKIWGAYDGKFEKTISGHKLGISDVAWSSDSNLLVSASDDKTLKIWDVSSGKCLKTLKGHSNYVFCCNFNPQSNLIVSGSFDESVRIWDVKTGKCLKTLPAHSDPVSAVHFNRDGSLIVSSSYDGLCRIWDTASGQCLKTLIDDDNPPVSFVKFSPNGKYILAATLDNTLKLWDYSKGKCLKTYTGHKNEKYCIFANFSVTGGKWIVSGSEDNLVYIWNLQTKEIVQKLQGHTDVVISTACHPTENIIASAALENDKTIKLWKSDC

SEQ ID NO: 16 - protein, 117 aa, Homo sapiens
GIDPFTMLPRLCCLEKGPNGYGFHLHGEKGKLGQYIRLVEPGSPAEKAGLLAGDRLVEVNGENVEKETHQQVVSRIRAALNAVRLLVVDPETDEQLQKLGVQVREELLRAQEAPGQA

SEQ ID NO: 17 - protein, 128 aa, Homo sapiens
GIDPFTMLRPRLCTMKKGPSGYGFNLHSDKSKPGQFIRSVDPDSPAEASGLRAQDRIVEVNGVCMEGKQHGDVVSAIRAGGDETKLLVVDRETDEFFKKCRVIPSQEHLNGPLPVPFTNGEIQKENSR

SEQ ID NO: 18 - protein, 303 aa, Homo sapiens
VKPNYALKFTLAGHTKAVSSVKFSPNGEWLASSSADKLIKIWGAYDGKFEKTISGHKLGISDVAWSSDSNLLVSASDDKTLKIWDVSSGKCLKTLKGHSNYVFCCNFNPQSNLIVSGSFDESVRIWDVKTGKCLKTLPAHSDPVSAVHFNRDGSLIVSSSYDGLCRIWDTASGQCLKTLIDDNPPVSFVKFSPNGKYILAATLDNTLKLWDYSKGKCLKTYTGHKNEKYCIFANFSVTGGKWIVSGSEDNLVYIWNLQTKEIVQKLQGHTDVVISTACHPTENIIASAALENDKTIKLWKSDC

SEQ ID NO: 19 - protein, 350 aa, Homo sapiens
  VARIANT 348: note = Any amino acid
  VARIANT 349: note = Any amino acid
  VARIANT 350: note = Any amino acid
ASESETLNPSARIMTFYPTMEEFRNFSRYIAYIESQGAHRAGLAKVVPPKEWKPRASYDDIDDLVIPAPIQQLVTGQSGLFTQYNIQKKAMTVREFRKIANSDKYCTPRYSEFEELERKYWKNLTFNPPIYGADVNGTLYEKHVDEWNIGRLRTILDLVEKESGITIEGVNTPYLYFGMWKTSFAWHTEDMDLYSINYLHFGEPKSWYSVPPEHGKRLERLAKGFFPGSAQSCEAFLRHKMTLISPLMLKKYGIPFDKVTQEAGEFMITFPYGYHAGFNHGFNCAESTNFATRRWIEYGKQAVLCSCRKDMVKISMDVFVRKFQPERYKLWKAGKDNTVIDHTLPTPXXX

SEQ ID NO: 20 - protein, 146 aa, Homo sapiens
  VARIANT 146: note = Any amino acid
LPPGWEKAMSRSSGRVYYFNHITNASQWERPSEPARVRCSHLLVKHSQSRRPSSWRQEKITRTKEEALELINGYIQKIKSGEEDFESLASQFSDCSSAKARGDLGAFSRGQMQKPFEDASFALRTGEMSGPVFTDSGIHIILRTEX

SEQ ID NO: 21 - protein, 233 aa, Homo sapiens
DCHLSDMLQQLHSVNASKPSERGLVRQEEAEDPACIPIFWVSKWVDYSDKYGLGYQLCDNSVGVLFNDSTRLILYNDGDSLQYIERDGTESYLTVSSHPNSLMKKITLLKYFRNYMSEHLLKAGANITPREGDELARLPYLRTWFRTRSAIILHLSNGSVQINFFQDHTKLILCPLMAAVTYIDEKRDFRTYRLSLLEEYGCCKELASRLRYARTMVDKLLSSRSASNRLKAS

SEQ ID NO: 22 - protein, 229 aa, Homo sapiens
DKVYQMKSKPRGYCLIINNHNFAKAREKVPKLHSIRDRNGTHLDAGALTTTFEELHFEIKPHDDCTVEQIYEILKIYQLMDHSNMDCFICCILSHGDKGIIYGTDGQEAPIYELTSQFTGLKCPSLAGKPKVFFIQACQGDNYQQTRYIPDEADFLLGMATVNNCVSYRNPAEGTWYIQSLCQSLRERCPRGDDILTILTEVNYEVSNKGKQMPQPTFTLRKKLVFPSD

SEQ ID NO: 23 - protein, 213 aa, Homo sapiens [sequence omitted]

SEQ ID NO: 24 - protein, 98 aa, Homo sapiens
MKPHPWFFGKIPRAKAEEMLSKQRHDGAFLIRESESAPGDFSLSVKFGNDVQHFKVLRDGAGKYFLWVVKFNSLNELVDYHRSTSVSRNQQIFLRDIE

SEQ ID NO: 25 - protein, 84 aa, Homo sapiens
FKFHSGEKVLCFEPDPTKARVLYDAKIVDVIVGKDEKGRKIPEYLIHFNGWNRSWDRWAAEDHVLRDTDENRRLQRKLARKAVA

SEQ ID NO: 26 - protein, 212 aa, Homo sapiens
HLSDMLQQLHSVNASKPSERGLVRQEEAEDPACIPIFWVSKWVDYSDKYGLGYQLCDNSVGVLFNDSTRLILYNDGDSLQYIERDGTESYLTVSSHPNSLMKKITLLKYFRNYMSEHLLKAGAPRLPYLRTWFRTRSAIILHLSNGSVQINFFQDHTKLILCPLMAAVTYIDEKRDFRTYRLSLLEEYGCCKELASRLRYARTMVDKLLSSR

SEQ ID NO: 27 - protein, 221 aa, Homo sapiens
  VARIANT 220: note = Any amino acid
  VARIANT 221: note = Any amino acid
HLSDMLQQLHSVNASKPSERGLVRQEEAEDPACIPIFWVSKWVDYSDKYGLGYQLCDNSVGVLFNDSTRLILYNDGDSLQYIERDGTESYLTVSPNSLMKKITLLKYFRNYMSEHLLKAGANITPREGDELARLPYLRTWFRTRSAIILHLSNGSVQINFFQDHTKLILCPLMAAVTYIDEKRDFRTYRLSLLEEYGCCKELASRLRYARTMVDKLLSSXX

SEQ ID NO: 28 - protein, 223 aa, Homo sapiens [sequence omitted]

SEQ ID NO: 29 - protein, 309 aa, Homo sapiens
TQSKPTPVKPNYALKFTLAGHTKAVSSVKFSPNGEWLASSSADKLIKIWGAYDGKFEKTISGHKLGISDVAWSSDSNLLVSASDDKTLKIWDVSSGKCLKTLKGHSNYVFCCNFNPQSNLIVSGSFDESVRIWDVKTGKCLKTLPAHSDPVSAVHFNRDGSLIVSSSYDGLCRIWDTASGQCLKTLIDNPPVSFVKFSPNGKYILAATLDNTLKLWDYSKGKCLKTYTGHKNEKYCIFANFSVTGGKWIVSGSEDNLVYIWNLQTKEIVQKLQGHTDVVISTACHPTENIIASAALENDKTIKLWKSDC

SEQ ID NO: 30 - protein, 60 aa, Homo sapiens
  VARIANT 56: note = Any amino acid
  VARIANT 57: note = Any amino acid
  VARIANT 58: note = Any amino acid
  VARIANT 59: note = Any amino acid
  VARIANT 60: note = Any amino acid
LLQAQALYPWRAKKDNHLNFNKNDVITVLEQQDMWWFGEVQGQKGWFPKSYVKLIXXXXX

SEQ ID NO: 31 - protein, 87 aa, Homo sapiens
GPIRKVLLLKEDHEGLGISITGGKEHGVPILISEIHPGQPADRCGGLHVGDAILAVNGVNLRDTKHKEAVTILSQQRGEIEFEVVYV

SEQ ID NO: 32 - protein, 227 aa, Homo sapiens
  VARIANT 227: note = Any amino acid
ADNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTKIPVEADFLYAYSTAPGYYSWRNSKDGSWFIQSLCAMLKQYADKLEFMHILTRVNRKVATEFESFSFDATFHAKKQIPCIVSMLTKELYFYHX

SEQ ID NO: 33 - protein, 365 aa, Homo sapiens
  VARIANT 365: note = Any amino acid
HHPPSYVAHLASDFGVRVFQQVAQASKDRNVVFSPYGVASVLAMLQLTTGGETQQQIQAAMGFKIDDKGMAPALRHLYKELMGPWNKDEISTTDAIFVQRDLKLVQGFMPHFFRLFRSTVKQVDFSEVERARFIINDWVKTHTKGMISNLLGKGAVDQLTRLVLVNALYFNGQWKTPFPDSSTHRRLFHKSDGSTVSVPMMAQTNKFNYTEFTTPDGHYYDILELPYHGDTLSMFIAAPYEKEVPLSALTNILSAQLISHWKGNMTRLPRLLVLPKFSLETEVDLRKPLENLGMTDMFRQFQADFTSLSDQEPLHVAQALQKVKIEVNESGTAPEEIIMDRPFLFVVRHNPTGTVLFMGQVMEPX

SEQ ID NO: 34 - protein, 164 aa, Homo sapiens; identical to SEQ ID NO: 11

SEQ ID NO: 35 - protein, 98 aa, Homo sapiens; identical to SEQ ID NO: 24

SEQ ID NO: 36 - protein, 96 aa, Homo sapiens
  VARIANT 96: note = Any amino acid
PWAVKPEDKAKYDAIFDSLSPVNGFLSGDKVKPVLLNSKLPVDILGRVWELSDIDHDGMLDRDEFAVAMFLVYCALEKEPVPMSLPPALVPPSKRX

SEQ ID NO: 37 - protein, 306 aa, Homo sapiens
  VARIANT 178: note = Any amino acid
  VARIANT 182: note = Any amino acid
  VARIANT 183: note = Any amino acid
  VARIANT 304: note = Any amino acid
  VARIANT 305: note = Any amino acid
  VARIANT 306: note = Any amino acid
SSVFVPDEWEVSREKITLLRELGQGSFGMVYEGNARDIIKGEAETRVAVKTVNESASLRERIEFLNEASVMKGFTCHHVVRLLGVVSKGQPTLVVMELMAHGDLKSYLRSLRPEAENNPGRPPPTLQEMIQMAAEIADGMAYLNAKKFVHRDLAARNCMVAHDFTVKIGDFGMTRDIXETDXXRKGGKGLLPVRWMAPESLKDGVFTTSSDMWSFGVVLWEITSLAEQPYQGLSNEQVLKFVMDGGYLDQPDNCPERVTDLMRMCWQFNPKMRPTFLEIVNLLKDDLHPSFPEVSFFHSEENKXXX

SEQ ID NO: 38 - protein, 223 aa, Homo sapiens
MERASLIQKAKLAEQAERYEDMAAFMKGAVEKGEELSCEERNLLSVAYKNVVGGQRAAWRVLSSIEQKSNPEVREYREKVETELQGVCDTVLGLLDSHLIKEAGDAESRVFYLKMKGDYYRYLAEVATGDDKKRIIDSARSAYQEAMDISKKEMPPTNPIRLGLALNFSVFHYEIANSPEEAISLAKTTFDEAMADLHTLSEDSYKDSTLIMQLLRDNLTLWT

SEQ ID NO: 39 - protein, 92 aa, Homo sapiens
MEIKLIKGPKGLGFSIAGGVGNQHIPGDNSIYVTKIIEGGAAHKDGKLQIGDKLLAVNNVCLEEVTHEEAVTALKNTSDFVYLKVAKPTGSH

SEQ ID NO: 40 - protein, 132 aa, Homo sapiens
GAHMAGGRSWCLRRVGMSAGWLLLEDGCEVTVGRGFGVTYQLVSKICPLMISRNHCVLKQNPEGQWTIMDNKSLNGVWLNRARLEPLRVYSIHQGDYIQLGVPLENKENAEYEYEVTEEDWETIYPCLSPKN

SEQ ID NO: 41 - protein, 76 aa, Homo sapiens
  VARIANT 72: note = Any amino acid
  VARIANT 73: note = Any amino acid
  VARIANT 74: note = Any amino acid
  VARIANT 75: note = Any amino acid
  VARIANT 76: note = Any amino acid
GSMATVPVYCVCRLPYDVTRFMIECDACKDWFHGSCVGVEEEEAPDIDIYHCPNCEKTHGKSTLKKKRTWHXXXXX

SEQ ID NO: 42 - protein, 100 aa, Homo sapiens
RRWFHPNITGVEAENLLLTRGVDGSFLARPSKSNPGDFTLSVRRNGAVTHIKIQNTGDYYDLYGGEKFATLAELVQYYMEHHGQLKEKNGDVIELKYPLN

SEQ ID NO: 43 - protein, 234 aa, Homo sapiens
DQPLAKDKVALLIGNMNYREHPKLKAPLVDVYELTNLLRQLDFKVVSLLDLTEYEMRNAVDEFLLLLDKGVYGLLYYAGHGYENFGNSFMVPVDAPNPYRSENCLCVQNILKLMQEKETGLNVFLLDMCRKRNDYDDTIPILDALKVTANIVFGYATCQGAEAFEIQHSGLANGIFMKFLKDRLLEDKKITVLLDEVAEDMGKCHLTKGKQALEIRSSLSEKRALTDPIQGTEY

SEQ ID NO: 44 - protein, 293 aa, Homo sapiens
  VARIANT 175: note = Any amino acid
  VARIANT 291: note = Any amino acid
  VARIANT 292: note = Any amino acid
  VARIANT 293: note = Any amino acid
SHEQFRAALQLVVDPGDPRSYLDNFIKIGEGSTGIVCIATVRSSGKLVAVKKMDLRKQQRRELLFNEVVIMRDYQHENVVEMYNSYLVGDELWVVMEFLEGGALTDIVTHTRMNEEQIAAVCLAVLQALSVLHAQGVIHRDIKSDSILLTHDGRVKLSDFGFCAQVSKEVPRRKXLVGTPYWMAPELISRLPYGPEVDIWSLGIMVIEMVDGEPPYFNEPPLKAMKMIRDNLPPRLKNLHKVSPSLKGFLDRLLVRDPAQRATAAELLKHPFLAKAGPPASIVPLMRQNRXXX

SEQ ID NO: 45 - protein, 87 aa, Homo sapiens; identical to SEQ ID NO: 31

SEQ ID NO: 46 - protein, 111 aa, Homo sapiens
TAVVQRVEIHKLRQGENLILGFSIGGGIDQDPSQNPFSEDKTDKGIYVTRVSEGGPAEIAGLQIGDKIMQVNGWDMTMVTHDQARKRLTKRSEEVVRLLVTRQSLQKAVQQ

SEQ ID NO: 47 - protein, 88 aa, Homo sapiens
  VARIANT 88: note = Any amino acid
GPIRKVLLLKEDHEGLGISITGGKEHGVPILISEIHPGQPADRCGGLHVGDAILAVNGVNLRDTKHKEAVTILSQQRGEIEFEVVYVX

SEQ ID NO: 48 - protein, 155 aa, Homo sapiens
GSHGMADEEKLPPGWEKRMSRSSGRVYYFNHITNASQWERPSEPARVRCSHLLVKHSQSRRPSSWRQEKITRTKEEALELINGYIQKIKSGEEDFESLASQFSDCSSAKARGDLGAFSRGQMQKPFEDASFALRTGEMSGPVFTDSGIHIILRTE

SEQ ID NO: 49 - protein, 146 aa, Homo sapiens
  VARIANT 143: note = Any amino acid
  VARIANT 144: note = Any amino acid
  VARIANT 145: note = Any amino acid
  VARIANT 146: note = Any amino acid
QLTEEQIAEFKEAFSLFDKDGDGTITTKELGTVMRSLGQNPTEAELQDMINEVDADGNGTIDFPEFLTMMARKMKDSEEEIREAFRVFDKDGNGYISAAELRHVMTNLGEKLTDEEVDEMIREADIDGDGQVNYEEFVQMMTXXXX

SEQ ID NO: 50 - protein, 110 aa, Homo sapiens
LQSITAGQKVISKHKNGRFYQCEVVRLTTETFYEVNFDDGSFSDNLYPEDIVSGPPAEGEVVQVRWTDGQVYGAKFVASHPIQMYQVEFEDGSQLVVKRDDVYTLDEELP

SEQ ID NO: 51 - protein, 442 aa, Homo sapiens
TQVKHMMQVIEPQFQRDFISLLPKELALYVLSFLEPKDLLQAAQTCRYWRILAEDNLLWREKCKEEGIDEPLHIKRVIKPGFIHSPWKSAYIRQHRIDTNWRRGELKSPKVLKGHDDHVITCLQFCGNRIVSGSDDNTLKVWSAVTGKCLRTLVGHTGGVWSSQMRDNIIISGSTDRTLKVWNAETGECIHTLYGHTSTVRCMHLHEKRVVSGSRDATLRVWDIETGQCLHVLMGHVAAVRCVQYDGRRVVSGAYDFMVKVWDPETETCLHTLQGHTNRVYSLQFDGIHVVSGSLDTSIRVWDVETGNCIHTLTGHQSLTSGMELKDNILVSGNADSTVKIWDIKTGQCLQTLQGPNKHQSAVTCLQFNKNFVITSSDDGTVKLWDLKTGEFIRNLVTLESGGSGGVVWRIRASNTKLVCAVGSRNGTEETKLLVLDFDVDM

SEQ ID NO: 52 - protein, 274 aa, Homo sapiens
  VARIANT 274: note = Any amino acid
PLESQYQVGPLLGSGGFGSVYSGIRVSDNLPVAIKHVEKDRISDWGELPNGTRVPMEVVLLKKVSSGFSGVIRLLDWFERPDSFVLILERPEPVQDLFDFITERGALQEELARSFFWQVLEAVRHCHNCGVLHRDIKDENILIDLNRGELKLIDFGSGALLKDTVYTDFDGTRVYSPPEWIRYHRYHGRSAAVWSLGILLYDMVCGDIPFEHDEEIIGGQVFFRQRVSSECQHLIRWCLALRPSDRPTFEEIQNHPWMQDVLLPQETAEIHLHX

SEQ ID NO: 53 - protein, 87 aa, Homo sapiens; identical to SEQ ID NO: 31

SEQ ID NO: 54 - protein, 228 aa, Homo sapiens
ENVQDLLLLDVAPLSLGLETAGGVMTALIKRNSTIPTKQTQIFTTYSDNQPGVLIQVYEGERAMTKDNNLLGRFELSGIPPAPRGVPQIEVTFDIDANGILNVTATDKSTGKANKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISGLYQG

SEQ ID NO: 55 - protein, 98 aa, Homo sapiens
GSWFFKNLSRKDAERQLLAPGNTHGSFLIRESESTAGSFSLSVRDFDQNQGEVVKHYKIRNLDNGGFYISPRITFPGLHELVRHYTNASDGLCTRLSR

SEQ ID NO: 56 - protein, 189 aa, Homo sapiens
SPELGFSISGGVGGRGNPFRPDDDGIFVTRVQPEGPASKLLQPGDKIIQANGYSFINIEHGQAVSLLKTFQNTVELIIVREVGNGAKQEIRVRVEKDSSVPTNLEVVAATPTSLLISWDASYYGVSYYRITYGETGGNSPVQEFTVPYSSSTATISGLKPGVDYTITVYAYSDYYGSHHYSPISINYRT

SEQ ID NO: 57 - protein, 191 aa, Homo sapiens
  VARIANT 191: note = Any amino acid
SPELGFSISGGVGGRGNPFRPDDDGIFVTRVQPEGPASKLLQPGDKIIQANGYSFINIEHGQAVSLLKTFQNTVELIIVREGAKQEIRVRVEKDGGSGGVSSVPTNLEVVAATPTSLLISWDAYRELPVSYYRITYGETGGNSPVQEFTVPGSKSTATISGLKPGVDYTITVYAHYNYHYYSSPISINYRX

SEQ ID NO: 58 - protein, 285 aa, Homo sapiens
  VARIANT 281: note = Any amino acid
  VARIANT 282: note = Any amino acid
  VARIANT 283: note = Any amino acid
  VARIANT 284: note = Any amino acid
  VARIANT 285: note = Any amino acid
GSNSQVWSALQMSKALPSPVERIVSRDIARGYERIPIPCVNAVDSEPCPSNYKYVSQNCVTSPMNIDRNITHLQYCVCIDDCSSSNCMCGQLSMRCWYDKDGRLLPEFNMAEPPLIFECNHACSCWRNCRNRVVQNGLRARLQLYRTRDMGWGVRSLQDIPPGTFVCEYVGELISDSEADVREEDSYLFDLDNKDGEVYCIDARFYGNVSRFINHHCEPNLVPVRVFMAHQDLRFPRIAFFSTRLIEAGEQLGFDYGERFWDIKGKLFSCRCGSPKCRHSXXXXX

SEQ ID NO: 59 - protein, 102 aa, Homo sapiens
AVVQRVEIHKLRQGENLILGFSIGGGIDQDPSQNPFSEDKTDKGIYVTRVSEGGPAEIAGLQIGDKIMQVNGWDMTMVTHDQARKRLTKRSEEVVRLLVTRQ

SEQ ID NO: 60 - protein, 240 aa, Homo sapiens
  VARIANT 240: note = Any amino acid
PIFLNVLEAIEPGVVCAGHDNNQPDSFAALLSSLNELGERQLVHVVKWAKALPGFRNLHVDDQMAVIQYSLMGLMVFAMGWRSFTNVNSAMLYFAPDLVFNEYRMHKSRMYSQCVRMRHLSQEFGWLQITPQEFLCMKALLLFSIIPVDGLKNQKFFDELRMNYIKELDRIIACSRRFYQLTKLLDSVQPIARELHQFTFDLLIKSHMVSVDFPEMMAEIISVQVPKILSGKVKPIYFHX

SEQ ID NO: 61 - protein, 277 aa, Homo sapiens
GSHSMRYFYTAMSRPGRGEPRFIAVGYVDDTQFVRFDSDAASPRTEPRAPWIEQEGPEYWDRNTQIFKTNTQTYRESLRNLRGYYNQSEAGSHIIQRMYGCDLGPDGRLLRGHDQSAYDGKDYIALNEDLSSWTAADTAAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEPS

SEQ ID NO: 62 - protein, 138 aa, Homo sapiens
GWNAYIDNLMADGTCQDAAIVGYKDSPSVWAAVPGKTFVNITPAEVGVLVGKDRSSFYVNGLTLGGQKCSVIRDSLLQDGEFSMDLRTKSTGGAPTFNVTVTKTDKTLVLLMGKEGVHGGLINKKCYEMASHLRRSQY

SEQ ID NO: 63 - protein, 298 aa, Homo sapiens
  VARIANT 298: note = Any amino acid
EMEKEFEQIDKSGSWAAIYQDIRHEASDFPCRVAKLPKNKNRNRYRDVSPFDHSRIKLHQEDNDYINASLIKMEEAQRSYILTQGPLPNTCGHFWEMVWEQKSRGVVMLNRVMEKGSLKCAQYWPQKEEKEMIFEDTNLKLTLISEDIKSYYTVRQLELENLTTQETREILHFHYTTWPDFGVPESPASFLNFLFKVRESGSLSPEHGPVVVHSSAGIGRSGTFCLADTCLLLMDKRKDPSSVDIKKVLLEMRKFRMGLIQTADQLRFSYLAVIEGAKFIMGDSSVQDQWKELSHEDX

SEQ ID NO: 64 - protein, 276 aa, Homo sapiens
GSHSMRYFHTSVSRPGRGEPRFITVGYVDDTLFVRFDSDAASPREEPRAPWIEQEGPEYWDRETQICKAKAQTDREDLRTLLRYYNQSEAGSHTLQNMYGCDVGPDGRLLRGYHQDAYDGKDYIALNEDLSSWTAADTAAQITQRKWEAARVAEQLRAYLEGECVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 65 - protein, 83 aa, Homo sapiens
QVRPKLPLLKILHAAGAQGEMFTVKEVMHYLGQYIMVKQLYDQQEQHMVYCGGDLLGELLGRQSFSVKDPSPLYDMLRKNLVT

SEQ ID NO: 66 - protein, 276 aa, Homo sapiens
GSHSMRYFYTAMSRPGRGEPRFITVGYVDDTLFVRFDSDATSPRKEPRAPWIEQEGPEYWDRETQISKTNTQTYRENLRTALRYYNQSEAGSHIIQRMYGCDVGPDGRLLRGYDQYAYDGKDYIALNEDLSSWTAADTAAQITQRKWEAARVAEQDRAYLEGLCVESLRRYLENGKETLQRADPPKTHVTHHPISDHEVTLRCWALGFYPAEITLTWQRDGEDQTQDTELVETRPAGDRTFQKWAAVVVPSGEEQRYTCHVQHEGLPKPLTLRWEP

SEQ ID NO: 67 - protein, 172 aa, Homo sapiens
  VARIANT 165: note = Any amino acid
  VARIANT 166: note = Any amino acid
  VARIANT 167: note = Any amino acid
  VARIANT 168: note = Any amino acid
  VARIANT 169: note = Any amino acid
  VARIANT 170: note = Any amino acid
  VARIANT 171: note = Any amino acid
  VARIANT 172: note = Any amino acid
GFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVGYSLFLVAAHAFGHALGLDHSSVPEALMYPMYRFTEGPPLHKDDVNGIRHLYGXXXXXXXX

SEQ ID NO: 68 - protein, 121 aa, Homo sapiens
EDLIDGIIFAANYLGSTQLLSDKTPSKNVRQAQEAVSRIKAQKLTEVDLFILTQRIKVLNADTQETDHPLRTISYIADIGNIVVLARRRYKICHVFESEDAQLIAQSIGQAFSVAYQEFLR

SEQ ID NO: 69 - protein, 93 aa, Homo sapiens
  VARIANT 93: note = Any amino acid
EQETLVRPKPLLLKLLKSVGAQKDTYTMKEVLFYLGQYIMTKRLYDEKQQHIVYCSNDLLGDLFGVPSFSVKEHRKIYTMIYRNLVVVNQQEX

SEQ ID NO: 70 - protein, 231 aa, Homo sapiens
  VARIANT 231: note = Any amino acid
MDKNELVQKAKLAEQAERYDDMAACMKSVTEQGAELSNEERNLLSVAYKNVVGARRSSWRVVSSIEQKTEGAEKKQQMAREYREKIETELRDICNDVLSLLEKFLIPNASQAESKVFYLKMKGDYYRYLAEVAAGDDKKGIVDQSQQAYQEAFEISKKEMQPTHPIRLGLALNFSVFYYEILNSPEKACSLAKTAFDEAIAELDTLSEESYKDSTLIMQLLRDNLTLWTSX

SEQ ID NO: 71 - protein, 140 aa, Homo sapiens
AVSESQLKKMVSKYKYRDLTVRETVNVITLYKDLKPVLDSYGGSRELMNLTGTIPVPYRGNTYNIPICLWLLDTYPYNPPICFVKPTSSMTIKTGKHVDANGKIYLPYLHEWKHPQSDLLGLIQVMIVVFGDEPPVFSRP

SEQ ID NO: 72 - protein, 302 aa, Homo sapiens
  VARIANT 298: note = Any amino acid
  VARIANT 299: note = Any amino acid
  VARIANT 300: note = Any amino acid
  VARIANT 301: note = Any amino acid
  VARIANT 302: note = Any amino acid
GHMENFQKVEKIGEGTYGVVYKARNKLTGEVVALKKIRLDTETEGVPSTAIREISLLKELNHPNIVKLLDVIHTENKLYLVFEFLHQDLKKFMDASALTGIPLPLIKSYLFQLLQGLAFCHSHRVLHRDLKPQNLLINTEGAIKLADFGLARAFGVPVRTYHEVVTLWYRAPEILLGCKYYSTAVDIWSLGCIFAEMVTRRALFPGDSEIDQLFRIFRTLGTPDEVVWPGVTSMPDYKPSFPKWARQDFSKVVPPLDEDGRSLLSQMLHYDPNKRISAKAALAHPFFQDVTKPVPHLXXXXX

SEQ ID NO: 73 - protein, 103 aa, Homo sapiens
TAVVQRVEIHKLRQGENLILGFSIGGGIDQDPSQNPFSEDKTDKGIYVTRVSEGGPAEIAGLQIGDKIMQVNGWDMTMVTHDQARKRLTKRSEEVVRLLVTRQ

SEQ ID NO: 74 - protein, 60 aa, Homo sapiens
  VARIANT 59: note = Any amino acid
  VARIANT 60: note = Any amino acid
PNLFVALYDFVASGDNTLSITKGEKLRVLGYNQTGEWCEAQTKNGQGWVPSNYITPVNXX

SEQ ID NO: 75 - protein, 84 aa, Homo sapiens
  VARIANT 75: note = Any amino acid
NQVRPKLPLLKILHAAGAQGEMFTVKEVMHYLGQYIMVKQLYDQQEQHMVYCGGDLLGELLGRQSFSVKDPSPLXDMLRKNLVT

SEQ ID NO: 76 - protein, 127 aa, Homo sapiens
SPEFIILQTYRAIADYEKTSGSEMALSTGDVVEVVEKSESGWWFCQMKAKRGWIPASFLEPLDSPDEPNYAGEPYVAIKAYTAVEGDEVSLLEGEAVEVIHKLLDGWWVIRKDDVTGYFPSMYLQKS

SEQ ID NO: 77 - protein, 214 aa, Homo sapiens
ALQLTQSPSSLSASVGDRITITCRASQGVTSALAWYRQKPGSPPQLLIYDASSLESGVPSRFSGSGSGTEFTLTISTLRPEDFATYYCQQLHFYPHTFGGGTRVDVRRTVAAPSVFIFPPSDEQLKSGTASVVCLLNNFYPREAKVQWKVDNALQSGNSQESVTEQDSKDSTYSLSSTLTLSKADYEKHKVYECEVTHQGLSSPVTKSFNRGEC

SEQ ID NO: 78 - protein, 270 aa, Homo sapiens
  VARIANT 270: note = Any amino acid
HPICEVSKVASHLEVNCDKRQLTALPPDLPKDTTILHLSENLLYTFSLATLMPYTRLTQLNLDRCELTKLQVDGTLPVLGTLDLSHNQLQSLPLLGQTLPALTVLDVSFNRLTSLPLGALRGLGELQELYLKGNELKTLPPGLLTPTPKLEKLSLANNQLTELPAGLLNGLENLDTLLLQENSLYTIPKGFFGSHLLPFAFLHGNPWLCNCEILYFRRWLQDNAENVYVWKQGVDVKAMTSNVASVQCDNSDKFPVYKYPGKGCPLVPRX

SEQ ID NO: 79 - protein, 212 aa, Homo sapiens
  VARIANT 212: note = Any amino acid
RMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPX

SEQ ID NO: 80 - protein, 72 aa, Homo sapiens
  VARIANT 70: note = Any amino acid
  VARIANT 71: note = Any amino acid
  VARIANT 72: note = Any amino acid
SGPSCKHCKDDVNRLCRVCACHLCGGRQDPDKQLMCDECDMAFHIYCLDPPLSSVPSEDEWYCPECRNDXXX

SEQ ID NO: 81 - protein, 85 aa, Homo sapiens
  VARIANT 85: note = Any amino acid
NQVRPKLPLLKILHAAGAQGEMFTVKEVMHYLGQYIMVKQLYDAAAQHMVYCGGDLLGELLGRQSFSVKDPSPLYDMLRKNLVTX

SEQ ID NO: 82 - protein, 86 aa, Homo sapiens
GPLGSPGPNSHNSNTPGIREAGSEDIIVVALYDYEAIHHEDLSFQKGDQMVVLEESGEWWKARSLATRKEGYIPSNYVARVDSLET

SEQ ID NO: 83 - protein, 343 aa, Homo sapiens
  VARIANT 342: note = Any amino acid
  VARIANT 343: note = Any amino acid
VASGSGEPREEAGALGPAWDESQLRSYSFPTRPIPRLSQSDPRAEELIENEEPVVLTDTNLVYPALKWDLEYLQENIGNGDFSVYSASTHKFLYYDEKKMANFQNFKPRSNREEMKFHEFVEKLQDIQQRGGEERLYLQQTLNDTVGRKIVMDFLGFNWNWINKQQGKRGWGQLTSNLLLIGMEGNVTPAHYGEQQNFFAQIKGYKRCILFPPDQFECLYPYPVHHPCDRQSQVDFDNPDYERFPNFQNVVGYETVVGPGDVLYIPMYWWHHIESLLNGGITITVNFWYKGAPTPKRIEYPLKAHQKVAIMRNIEKMLGEALGNPQEVGPLLNTMIKGRYNXX

SEQ ID NO: 84 - protein, 640 aa, Homo sapiens
  VARIANT 632: note = Any amino acid
  VARIANT 633: note = Any amino acid
  VARIANT 634: note = Any amino acid
  VARIANT 635: note = Any amino acid
  VARIANT 636: note = Any amino acid
  VARIANT 637: note = Any amino acid
  VARIANT 638: note = Any amino acid
  VARIANT 639: note = Any amino acid
  VARIANT 640: note = Any amino acid
PMYSIITPNILRLESEETMVLEAHDAQGDVPVTVTVHDFPGKKLVLSSEKTVLTPATNHMGNVTFTIPANRGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSQPVPGQQMTLKIEGDHGARVVLVAVDKGVFVLNKKNKLTQSKIWDVVEKADIGCTPGSGKDYAGVFSDAGLTFTSSSGQQTAQRAELQCPQPXXXXXXXXX

SEQ ID NO: 85 - protein, 351 aa, Homo sapiens
  VARIANT 351: note = Any amino acid
AGPEMVRGQVFDVGPRYTNLSYIGEGAYGMVCSAYDNVNKVRVAIKKISPFEHQTYCQRTLREIKILLRFRHENIIGINDIIRAPTIEQMKDVYIVQDLMETDLYKLLKTQHLSNDHICYFLYQILRGLKYIHSANVLHRDLKPSNLLLNTTCDLKICDFGLARVADPDHDHTGFLTEYVATRWYRAPEIMLNSKGYTKSIDIWSVGCILAEMLSNRPIFPGKHYLDQLNHILGILGSPSQEDLNCIINLKARNYLLSLPHKNKVPWNRLFPNADSKALDLLDKMLTFNPHKRIEVEQALAHPYLEQYYDPSDEPIAEAPFKFDMELDDLPKEKLKELIFEETARFQPGYX

SEQ ID NO: 86 - protein, 94 aa, Homo sapiens
MEIKLIKGPKGLGFSIAGGVGNQHIPGDNSIYVTKIIEGGAAHKDGKLQIGDKLLAVNNVCLEEVTHEEAVTALKNTSDFVYLKVAKPTGSHHH

SEQ ID NO: 87 - protein, 88 aa, Homo sapiens; identical to SEQ ID NO: 47
  VARIANT 88: note = Any amino acid

SEQ ID NO: 88 - protein, 129 aa, Homo sapiens
GAMGKPFFTRNPSELKGKFIHTKLRKSSRGFGFTVVGGDEPDEFLQIKSLVLDGPAALDGKMETGDVIVSVNDTCVLGHTHAQVVKIFQSIPIGASVDLELCRGYPLPFDPDDPNTSLVTSVAILDKEP

SEQ ID NO: 89 - protein, 89 aa, Homo sapiens
EETCFDKYTGNTYRVGDTYERPKDSMIWDCTCIGAGRGRISCTIANRCHEGGQSYKIGDTWRRPHETGGYMLECVCLGNGKGEWTCKPI

SEQ ID NO: 90 - protein, 105 aa, Homo sapiens
GSAQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPE

SEQ ID NO: 91 - protein, 210 aa, Homo sapiens
DGKYAQKLFNDLFEDYSNALRPVEDTDKVLNVTLQITLSQIKDMDERNQILTAYLWIRQIWHDAYLTWDRDQYDGLDSIRIPSDLVWRPDIVLYNKADDEEPVNTNVVLRYDGLITWDAPAITKSSCVVDVTYFPFDNQQCNLTFGSWTYNGNQVDIFNALDSGDLSDFIEDVEWEVHGMPAVKNVISYGCCSEPYPDVTFTLLLKRRSH

SEQ ID NO: 92 - protein, 276 aa, Homo sapiens
  VARIANT 275: note = Any amino acid
  VARIANT 276: note = Any amino acid
GSHSMRYFYTAMSRPGRGEPRFIAVGYVDDTQFVRFDSDAASPRMAPRAPWIEQEGPEYWDRETQKYKRQAQTDRVSLRNLRGYYNQSEAGSHTLQRMYGCDVGPDGRLLRGHDQSAYDGKDYIALNEDLSSWTAADTAAQITQRKWEAAREAEQWRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWXX

SEQ ID NO: 93 - protein, 132 aa, Homo sapiens
KVVKFSYMWTINNFSFCREEMGEVIKSSTFSSKLKWCLRVNPKGLDEESKDYLSLYLLLVSCKSEVRAKFKFSILNAKGEETKAMESQRAYRFVQGKDWGFKKFIRRGFLLDEANGLLPDDKLTLFCEVSVV

SEQ ID NO: 94 - protein, 135 aa, Homo sapiens
KVVKFSYMWTINNFSFCREEMGEVIKSSTFSSGDKLKWCLRVNPKGLDEESKDYLSLYLLLVSCPSEVRAKFKFSILNAKGEETKAMESQRAYRFVQGKDWGFKKFIRRGFLLDEANGLLPDDKLTLFCEVSVVQ

SEQ ID NO: 95 - protein, 127 aa, Homo sapiens
  VARIANT 126: note = Any amino acid
  VARIANT 127: note = Any amino acid
EHPEFLKAGKEPGLQIWRVEKFDLVPVPTNLYGDFFTGDAYVILKTVQLRNGNLQYDLHYWLGNECSQDESGAAAIFTVQLDDYLNGRAVQHREVQGFESATFLGYFKSGLKYKKGGVASGFKHVXX

SEQ ID NO: 96 - protein, 204 aa, Homo sapiens
DYCKPTRLDLLLDMPPVSYDVQLLHSWNNNDRSLNVFVKEDDKLIFHRHPVAQSTDAIRGKVGYTRGLHVWQITWAMRQRGTHAVVGVATADAPLHSVGYTTLVGNNHESWGWDLGRNRLYHDGKNQPSKTYPAFLEPDETFIVPDSFLVALDMDDGTLSFIVDGQYMGVAFRGLKGKKLYPVVSAVWGHCEIRMRYLNGLDPE

SEQ ID NO: 97 - protein, 100 aa, Homo sapiens
  VARIANT 99: note = Any amino acid
  VARIANT 100: note = Any amino acid
SEWYYGNVTRHQAECALNERGVEGDFLIRDSESSPSDFSVSLKASGKNKHFKVQLVDNVYCIGQRRFHTMDELVEHYKKAPIFTSEHGEKLYLVRALQXX

SEQ ID NO: 98 - protein, 122 aa, Homo sapiens
SCIQFTRHASDVLLNLNRLRSRDILTDVVIVVSREQFRAHKTVLMACSGLFYSIFTDQLKCNLSVINLDPEINPEGFCILLDFMYTSRLNLREGNIMAVMATAMYLQMEHVVDTCRKFIKAS

SEQ ID NO: 99 - protein, 165 aa, Homo sapiens
  VARIANT 41: note = Any amino acid
DDWRAARSMHEFSAKDIDGHMVNLDKYRGFVSIVTNVASQXGKTEVNYTQLVDLHARYAERGLRILAFPSNQFGKQEPGSNEEIKEFAAGYNVKFDMFSKIEVNGDDAHPLWKWMKIQPKGKGILGNAIKWNFTKFLIDKNGVVVKRYGPMEEPLVIEKDLPHYF

SEQ ID NO: 100 - protein, 91 aa, Homo sapiens
PSQMEHAMETMMFTFHKFAGDKGYLTKEDLRVLMEKEFPGFLENQKDPLAVDKIMKDLDQCRDGKVGFQSFFSLIAGLTIACNDYFVVHMK

SEQ ID NO: 101 - protein, 245 aa, Homo sapiens
FDCGKPQVEPKKCPGVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDAGGPLVCFEKDKYILQGVTSWGLGCARPNKPGVYVRVSRFVTWIEGVMRNN

SEQ ID NO: 102 - protein, 311 aa, Homo sapiens
HIGNYRLLKTIGAKVKLARHILTGKEVAVKIIDKTQLNSSSLQKLFREVRIMKVLNHPNIVKLFEVIETEKTLYLVMEYASGGEVFDYLVAHGWMKEKEARAKFRQIVSAVQYCHQKFIVHRDLKAENLLLDADMNIKIADFGFSNEFTFGNKLDTFCGSPPYAAPELFQGKKYDGPEVDVWSLGVILYTLVSGSLPFDGQNLKELRERVLRGKYRIPFYMSTDCENLLKKFLILNPSKRGTLEQIMKDRWMNVGHEDDELKPYVEPLPDYKDPRRTELMVSMGYTREEIQDSLVGQRYNEVMATYLLLGY

SEQ ID NO: 103 - protein, 65 aa, Homo sapiens
  VARIANT 64: note = Any amino acid
  VARIANT 65: note = Any amino acid
GTGVTLFVALYDYEARTEDDLSFHKGEKFQILNSSEGDWWEARSLTTGETGYIPSNYVAPVDSXX

SEQ ID NO: 104 - protein, 167 aa, Homo sapiens
  VARIANT 167: note = Any amino acid
LRQAVMLPEGEDLNEWIAVNTVDFFNQINMLYGTITEFCTEASCPVMSAGPRYEYHWADPIKCSAPKYIDYLMTWVQDQLDDETLFPSKIGVPFPKNFMSVAKTILKRLFRVYAHIYHQHFDSVMQLQEEAHLNTSFKHFIFFVQEFNLIDRRELAPLQELIEKLGX

SEQ ID NO: 105 - protein, 260 aa, Homo sapiens
GSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVDHEATLRCWALSFYPAEILWQREDQTQDTEVETRPAGDGTFQKWAVVVPSGQEYTCHQHEGLPLTL

SEQ ID NO: 106 - protein, 211 aa, Homo sapiens
SYVLTQPPSVSVSPGQTARITCSGDKLGDKYASWYQQKPGQSPVLVIYQDNKRPSEIPARFSGSNSGNTATLTISGAQAMDEADYYCQAWDSNTGVFGTGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRG

SEQ ID NO: 107 - protein, 216 aa, Homo sapiens
  VARIANT 212: note = Any amino acid
  VARIANT 213: note = Any amino acid
  VARIANT 214: note = Any amino acid
  VARIANT 215: note = Any amino acid
  VARIANT 216: note = Any amino acid
ELTLTQSPATLSLSPGERATLSCRASQSVSSYLAWYQQKPGQAPRLLIYDASNRATGIPARFSGSGSGTDFTLTISSLEPEDFAVYYCQQRSNWITFGQGTRLEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGXXXXX

SEQ ID NO: 108 - protein, 124 aa, Homo sapiens
  VARIANT 124: note = Any amino acid
GSAPIPDLKVFEREGVQLNLSFIRPPENPALLLITITATNFSEGDVTHFICQAAVPKSLQLQLQAPSGNTVPARGGLPITQLFRILNPNKAPLRLKLRLTYDHFHQSVQEIFEVNNLPVESWQX

SEQ ID NO: 109 - protein, 250 aa, Homo sapiens
  VARIANT 250: note = Any amino acid
IVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKEGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFX

SEQ ID NO: 110 - protein, 252 aa, Homo sapiens
  VARIANT 250: note = Any amino acid
  VARIANT 251: note = Any amino acid
  VARIANT 252: note = Any amino acid
IVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKEGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFXXX

SEQ ID NO: 111 - protein, 252 aa, Homo sapiens
  VARIANT 252: note = Any amino acid
IVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFX

SEQ ID NO: 112 - protein, 253 aa, Homo sapiens
IVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 113 - protein, 253 aa, Homo sapiens; identical to SEQ ID NO: 112

SEQ ID NO: 114 - protein, 250 aa, Homo sapiens
  VARIANT 249: note = Any amino acid
  VARIANT 250: note = Any amino acid
IVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKEGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQXX

SEQ ID NO: 115 - protein, 277 aa, Homo sapiens
  VARIANT 277: note = Any amino acid
DCGLRPLFEKKSLEDKTERELLESYIIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKEGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGX

SEQ ID NO: 116 - protein, 251 aa, Homo sapiens
  VARIANT 251: note = Any amino acid
IVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKEGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGX

SEQ ID NO: 117 - protein, 147 aa, Homo sapiens
IVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKET

SEQ ID NO: 118 - protein, 109 aa, Homo sapiens
GSPIPHHDEKTWNVGSSNRNKAENLLRGKRDGTFLVRESSKQGCYACSVVVDGEVKHCVINKTATGYGFAEPYNLYSSLKELVLHYQHTSLVQHNDSLNVTLAYPVYAQ

SEQ ID NO: 119 - protein, 277 aa, Homo sapiens
  VARIANT 275: note = Any amino acid
  VARIANT 276: note = Any amino acid
  VARIANT 277: note = Any amino acid
LAHSKMVPIPAGVFTMGTDDPQIKQDGEAPARRVTIDAFYMDAYEVSNTEFEKFVNSTGYLTEAEKFGDSFVFEGMLAAAPWWLPVKGANWRHPEGPDSTILHRPDHPVLHVSWNDAVAYCTWAGKRLPTEAEWEYSCRGGLHNRLFPWGNKLQPKGQHYANIWQGEFPVTNTGEDGFQGTAPVDAFPPNGYGLYNIVGNAWEWTSDWWTVHHSVEETLNPKGPPSGKDRVKKGGSYMSHRSYCYRYRCAARSQNTPDSSASNLGFRCAADRLPXXX

SEQ ID NO: 120 - protein, 337 aa, Homo sapiens
DYFQGAMGSKPAYSFHVTADGQMQPVPFPPDALIGPGIPRHARQINTLNHGEVVCAVTISNPTRHVYTGGKGCVKVWDISHPGNKSPVSQLDCLNRDNYIRSCKLLPDGCTLIVGGEASTLSIWDLAAPTPRIKAELTSSAPACYALAISPDSKVCFSCCSDGNIAVWDLHNQTLVRQFQGHTDGASCIDISNDGTKLWTGGLDNTVRSWDLREGRQLQQHDFTSQIFSLGYCPTGEWLAVGMESSNVEVLHVNKPDKYQLHLHESCVLSLKFAYCGKWFVSTGKDNLLNAWRTPYGASIFQSKESSSVLSCDISVDDKYIVTGSGDKKATVYEVIY

SEQ ID NO: 121 - protein, 292 aa, Homo sapiens
  VARIANT 275: note = Any amino acid
  VARIANT 276: note = Any amino acid
  VARIANT 277: note = Any amino acid
  VARIANT 278: note = Any amino acid
  VARIANT 279: note = Any amino acid
  VARIANT 280: note = Any amino acid
  VARIANT 281: note = Any amino acid
  VARIANT 282: note = Any amino acid
  VARIANT 283: note = Any amino acid
  VARIANT 284: note = Any amino acid
  VARIANT 285: note = Any amino acid
  VARIANT 286: note = Any amino acid
  VARIANT 287: note = Any amino acid
  VARIANT 288: note = Any amino acid
  VARIANT 289: note = Any amino acid
  VARIANT 290: note = Any amino acid
  VARIANT 291: note = Any amino acid
  VARIANT 292: note = Any amino acid
LAHSKMVPIPAGVFTMGTDDPQIKQDGEAPARRVTIDAFYMDAYEVSNTEFEKFVNSTGYLTEAEKFGDSFVFEGMLAAAPWWLPVKGANWRHPEGPDSTILHRPDHPVLHVSWNDAVAYCTWAGKRLPTEAEWEYSCRGGLHNRLFPWGNKLQPKGQHYANIWQGEFPVTNTGEDGFQGTAPVDAFPPNGYGLYNIVGNAWEWTSDWWTVHHSVEETLNPKGPPSGKDRVKKGGSYMSHRSYCYRYRCAARSQNTPDSSASNLGFRCAADRLPXXXXXXXXXXXXXXXXXX

SEQ ID NO: 122 - protein, 240 aa, Homo sapiens
  VARIANT 240: note = Any amino acid
HGVCWIYYPDGGSLVGEVNEDGEMTGEKIAYVYPDERTALYGKFIDGEMIEGKLATLMSTEEGRPHFELMPGNSVYHFDKSTSSCISTNALLPDPYESERVYVAESLISSAGEGLFSKVAVGPNTVMSFYNGVRITHQEVDSRDWALNGNTLSLDEETVIDVPEPYNHVSKYCASLGHKANHSFTPNCIYDMFVHPRFGPIKCIRTLRAVEADEELTVAYGYEAPEWYQVELKAFQATQX

SEQ ID NO: 123 - protein, 962 aa, Homo sapiens
  VARIANT 962: note = Any amino acid
NNPAIKRIGNHITKSPEDKREYRGLELANGIKVLLISDPTTDKSSAALDVHIGSLSDPPNIAGLSHFCQHMLFLGTKKYPKENEYSQFLSEHAGSSNAFTSGEHTNYYFDVSHEHLEGALDRFAQFFLCPLFDESCKDREVNAVDSEHEKNVMNDAWRLFQLEKATGNPKHPFSKFGTGNKYTLETRPNQEGIDVRQELLKFHSAYYSSNLMAVCVLGRESLDDLTNLVVKLFSEVENKNVPLPEFPEHPFQEEHLKQLYKIVPIKDIRNLYVTFPIPDLQKYYKSNPGHYLGHLIGHEGPGSLLSELKSKGWVNTLVGGQKEGARGFMFFIINVDLTEEGLLHVEDIILHMFQYIQKLRAEGPQEWVFQECKDLNAVAFRFKDKERPRGYTSKIAGILHYYPLEEVLTAEYLLEEFRPDLIEMVLDKLRPENVRVAIVSKSFEGKTDRTEEWYGTQYKQEAIPDEVIKKWQNADLNGKFKLPTKNEFIPTNFEILPLEKEATPYPALIKDTAMSKLWFKQDDKFFLPKACLNFEFFSPFAYVDPLHCNMAYLYLELLKDSLNEYAYAAELAGLSYDLQNTIYGMYLSVKGYNDKQPILLKKIIEKMATFEIDEKRFEIIKEAYMRSLNNFRAEQPHQHAMYYLRLLMTEVAWTKDELKEALDDVTLPRLKAFIPQLLSRLHIEALLHGNITKQAALGIMQMVEDTLIEHAHTKPLLPSQLVRYREVQLPDRGWFVYQQRNEVHNNCGIEIYYQTDMQSTSENMFLELFCQIISEPCFNTLRTKEQLGYIVFSGPRRANGIQGLRFIIQSEKPPHYLESRVEAFLITMEKSIEDMTEEAFQKHIQALAIRRLDKPKKLSAECAKYWGEIISQQYNFDRDNTEVAYLKTLTKEDIIKFYKEMLAVDAPRRHKVSVHVLAREMDSCPVVGNLSQAPALPQPEVIQNMTEFKRGLPLFPLVKPHX

SEQ ID NO: 124 - protein, 233 aa, Homo sapiens
IIGGSSSLPGSHPWLAAIYIGDSFCAGSLVHTCWVVSAAHCFSHSPPRDSVSVVLGQHFFNRTTDVTQTFGIEKYIPYTLYSVFNPSDHDLVLIRLKKKGDRCATRSQFVQPICLPEPGSTFPAGHKCQIAGWGHLDVSGYSSSLREALVPLVADHKCSSPEVYGADISPNMLCAGYFDCKSDACQGDSGGPLACEKNGVAYLYGIISWGCLHKPGVYTRVANYVDWINDRIR

SEQ ID NO: 125 - protein, 180 aa, Homo sapiens
  VARIANT 179: note = Any amino acid
  VARIANT 180: note = Any amino acid
PNEDWCAVCQNGGELLCCEKCPKVFHLSCHVPTLTNFPSGEWICTFCRDLSKPEVEYDCDAEKKKTEGLVKLTPIDKRKCERLLLFLYCHEMSLAFQDPVPLTVPDYYKIIKNPMDLSTIKKRLQEDYSMYSKPEDFVADFRLIFQNCAEFNEPDSEVANAGIKLENYFEELLKNLYPXX

SEQ ID NO: 126 - protein, 92 aa, Homo sapiens
  VARIANT 91: note = Any amino acid
  VARIANT 92: note = Any amino acid
PHAFKPGDLVFAKMKGYPHWPARIDDIAAVKPPPNKYPIFFFGTHETAFLGPKDLFPYDKCKDKYGKPNKRKGFNEGLWEIQNNPHASYSXX

SEQ ID NO: 127 - protein, 165 aa, Homo sapiens
IGPEDVLGLQRITGDYLCSPEENIYKIDFVRFKIRDMDSGTVLFEIKKPPNAGRFVRYQFTPAFLRLRQVGATVEFTVGDKPVNNFRMIERHYFRNQLLKSFDFHFGFCIPSSKNTCEHIYDFPPLSEELISEMIRHPYETQSDSFYFVDDRLVMHNKADYSYSG

SEQ ID NO: 128 - protein, 92 aa, Homo sapiens
MYEYEEITLERGNSGLGFSIAGGTDNPHIGDDSSIFITKIITGGAAAQDGRLRVNDCILRVNEVDVRDVTHSKAVEALKEAGSIVRLYVKRR

SEQ ID NO: 129 - protein, 285 aa, Homo sapiens
  VARIANT 285: note = Any amino acid
EEARALGRAVRMLQRLEEQCVVSPPSLRDLLPRTAQLLREVAHSRRAGGPGGPGGSGDFLLIYLANLEAKSRQVAALLSRLRRQLAKLAIIFSHMHAELHALFPGGKYCGHMYQLTKAPAHTFWRESCGARCVLPWAEFESLLGTCHPVEPGCTALALRTTIDLTCSGHVSIFEFDVFTRLFQPWPTLLKNWQLLAVNHPGYMAFLTYDEVQERLQACRDKPGSYIFRPSCTRLGQWAIGYVSSDGSILQTIPANKPLSQVLLEGQKDGFYLYPDGKTHNPDLTX

SEQ ID NO: 130 - protein, 474 aa, Homo sapiens
  VARIANT 470: note = Any amino acid
  VARIANT 471: note = Any amino acid
  VARIANT 472: note = Any amino acid
  VARIANT 473: note = Any amino acid
  VARIANT 474: note = Any amino acid
NICTTRGVSSCQQCLAVSPMCAWCSDEALPLGSPRCDLKENLLKDNCAPESIEFPVSEARVLEDRPLSDKGSGDSSQVTQVSPQRIALRLRPDDSKNFSIQVRQVEDYPVDIYYLMDLSYSMKDDLWSIQNLGTKLATQMRKLTSNLRIGFGAFVDKPVSPYMYISPPEALENPCYDMKTTCLPMFGYKHVLTLTDQVTRFNEEVKKQSVSRNRDAPEGGFDAIMQATVCDEKIGWRNDASHLLVFTTDAKTHIALDGRLAGIVQPNDGQCHVGSDNHYSASTTMDYPSLGLMTEKLSQKNINLIFAVTENVVNLYQNYSELIPGTTVGVLSMDSSNVLQLIVDAYGKIRSKVELEVRDLPEELSLSFNATCLNNEVIPGLKSCMGLKIGDTVSFSIEAKVRGCPQEKEKSFTIKPVGFKDSLIVQVTFDCDCACQAQAEPNSHRCNNGNGTFECGVCRCGPGWLGSQCXXXXX

SEQ ID NO: 131 - protein, 70 aa, Homo sapiens
  VARIANT 66: note = Any amino acid
  VARIANT 67: note = Any amino acid
  VARIANT 68: note = Any amino acid
  VARIANT 69: note = Any amino acid
  VARIANT 70: note = Any amino acid
PSCKHCKDDVNRLCRVCACHLCGGRQDPDKQLMCDECDMAFHIYCLDPPLSSVPSEDEWYCPECRXXXXX

SEQ ID NO: 132 - protein, 186 aa, Homo sapiens
  VARIANT 185: note = Any amino acid
  VARIANT 186: note = Any amino acid
KKIFKPEELRQALMPTLEALYRQDPESLPFRQPVDPQLLGIPDYFDIVKNPMDLSTIKRKLDTGQYQEPWQYVDDVWLMFNNAWLYNRKTSRVYKFCSKLAEVFEQEIDPVMQSLGYCCGRKYEFSPQTLCDDPSQPQTTKKKNDTLDPEPFVDCKECGRKMHQICVLHYDIIWPSGFVCDNCLXX

SEQ ID NO: 133 - protein, 299 aa, Homo sapiens
NFIIPKKEIHTVPDMGKWKRSQAYADYIGFILTLNEGVKGKKLTFEYRVSEAIEKLVALLNTLDRWIDETPPVDQPSRFGNKAYRTWYAKLDEEAENLVATVVPTHLAAAVPEVAVYLKESVGNSTRIDYGTGHEAAFAAFLCCLCKIGVLRVDDQIAIVFKVFNRYLEVMRKLQKTYRMEPAGGVWGLDDFQFLPFIWGSSQLIDHPYLEPRHFVDEKAVNENHKDYMFLECILFITEMKTGPFAEHSNQLWNISAVPSWSKVNQGLIRMYKAECLEKFPVIQHFKFGSLLPIHPVTS

SEQ ID NO: 134 - protein, 252 aa, Homo sapiens
FEARLVQGSILKKVLEALKDLINEACWDISSSGVNLQSMDSSHVSLVQLTLRSEGFDTYRCDRNLAMGVNLTSMSKILKCAGNEDIITLRAEDNADTLALVFEAPNQEKVSDYEMKLMDLDVEQLGIPEQEYSCVVKMPSGEFARICRDLSHIGDAVVISCAKDGVKFSASGELGNGNIKLSQTSNVDKEEEAVTIEMNEPVQLTFALRYLNFFTKATPLSSTVTLSMSADVPLVVEYKIADMGHLKYYLAP

SEQ ID NO: 135 - protein, 275 aa, Homo sapiens
  VARIANT 163: note = Any amino acid
  VARIANT 164: note = Any amino acid
  VARIANT 275: note = Any amino acid
VYLDRKLLTLEDKELGSGNFGTVKKGYYQMKKVVKTVAVKILKNEANDPALKDELLAEANVMQQLDNPYIVRMIGICEAESWMLVMEMAELGPLNKYLQQNRHVKDKNIIELVHQVSMGMKYLEESNFVHRDLAARNVLLVTQHYAKISDFGLSKALRADENXXKAQTHGKWPVKWYAPECINYYKFSSKSDVWSFGVLMWEAFSYGQKPYRGMKGSEVTAMLEKGERMGCPAGCPREMYDLMNLCWTYDVENRPGFAAVELRLRNYYYDVVNHX

SEQ ID NO: 136 - protein, 227 aa, Homo sapiens
  VARIANT 226: note = Any amino acid
  VARIANT 227: note = Any amino acid
MDKNELVQKAKLAEQAERYDDMAACMKSVTEQGAELSNEERNLLSVAYKNVVGARRSSWRVVSSIEQKAEKKQQMAREYREKIETELRDICNDVLSLLEKFLIPNASQAESKVFYLKMKGDYYRYLAEVAADDKKGIVDQSQQAYQEAFEISKKEMQPTHPIRLGLALNFSVFYYEILNSPEKACSLAKTAFDEAIAELDTLSEESYKDSTLIMQLLRDNLTLWTXX

SEQ ID NO: 137 - protein, 147 aa, Homo sapiens
  VARIANT 147: note = Any amino acid
SGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIETX

SEQ ID NO: 138 - protein, 194 aa, Homo sapiens
ENLYFQSMPEGLEELLSAPPPDLGAQRRHGWNPKDCSENIEVKEGGLYFERRPVAQSTDGARGKRGYSRGLHAWEISWPLEQRGTHAVVGVATALAPLQTDHYAALLGSNSESWGWDIGRGKLYHQSKGPGAPQYPEVPERLLVVLDMEEGTLGYAIGGTYLGPAFRGLKGRTLYPAVSAVWGQCQVRIRYLGE

SEQ ID NO: 139 - protein, 234 aa, Homo sapiens
DEATWERMWKHVAKIHPDGEKVAQRIRGATDLPKIPIPSVPTFQPSTPVPERLEAVQRYIRELQYNHTGTQFFEIKKSRPLTGLMDLAKEMTKEALPIKCLEAVILGIYLTNSMPTLERFPISFKTYFSGNYFRHIVLGVNFAGRYGALGMSRREDLMYKPPAFRTLSELVLDFEAAYGRCWHVLKKVKLGQSVSHDPHSVEQIEWKHSVLDVERLGRDDFRKELERHARDMRL

SEQ ID NO: 140 - protein, 112 aa, Homo sapiens
GSRRASVGSHEKMPWFHGKISREESEQIVLIGSKTNGKFLIRARDNNGSYALCLLHEGKVLHYRIDKDKTGKLSIPEGKKFDTLWQLVEHYSYKADGLLRVLTVPCQKIGTQ

SEQ ID NO: 141 - protein, 98 aa, Homo sapiens
  VARIANT 96: note = Any amino acid
  VARIANT 97: note = Any amino acid
  VARIANT 98: note = Any amino acid
GPAFPGMGSEELRLASFYDWPLTAEVPPELLAAAGFFHTGHQDKVRCFFCYGGLQSWKRGDDPWTEHAKWFPGCQFLLRSKGQEYINNIHLTHSLXXX

SEQ ID NO: 142 - protein, 93 aa, Homo sapiens
MKEPEIITVTLKKQNGMGLSIVAAKGAGQDKLGIYVKSVVKGGAADVDGRLAAGDQLLSVDGRSLVGLSQERAAELMTRTSSVVTLEVAKQGA

SEQ ID NO: 143 - protein, 230 aa, Homo sapiens
DREDLVYQAKLAEQAERYDEMVESMKKVAGMDVELTVEERNLLSVAYKNVIGARRASWRIISSIEQKEENKGGEDKLKMIREYRQMVETELKLICCDILDVLDKHLIPAANTGESKVFYYKMKGDYHRYLAEFATGNDRKEAAENSLVAYKAASDIAMTELPPTHPIRLGLALNFSVFYYEILNSPDRACRLAKAAFDDAIAELDTLSEESYKDSTLIMQLLRDNLTLWT

SEQ ID NO: 144 - protein, 164 aa, Homo sapiens
CPGIVPRSVWGARETHCPRMTLPAKYGIIIHTAGRTCNISDECRLLVRDIQSFYIDRLKSCDIGYNFLVGQDGAIYEGVGWNVQGSSTPGYDDIALGITFMGTFTGIPPNAAALEAAQDLIQCAMVKGYLTPNYLLVGHSDVARTLSPGQALYNIISTWPHFKH

SEQ ID NO: 145 - protein, 171 aa, Homo sapiens
  VARIANT 169: note = Any amino acid
  VARIANT 170: note = Any amino acid
  VARIANT 171: note = Any amino acid
TKLYCICKTPEDESKFYIGCDRCQNWYHGRCVGILQSEAELIDEYVCPQCQSTEDAMTVLTPLTEKDYEGLKRVLRSLQAHKMAWPFLEPVDPNDAPDYYGVIKEPMDLATMEERVQRRYYEKLTEFVADMTKIFDNCRYYNPSDSPFYQCAEVLESFFVQKLKGFKAXXX

SEQ ID NO: 146 - protein, 240 aa, Homo sapiens
  VARIANT 43: note = Any amino acid
  VARIANT 237: note = Any amino acid
  VARIANT 238: note = Any amino acid
  VARIANT 239: note = Any amino acid
  VARIANT 240: note = Any amino acid
GAMGSMERASLIQKAKLAEQAERYEDMAAFMKGAVEKGEELSXEERNLLSVAYKNVVGGQRAAWRVLSSIEQKSNEEGSEEKGPEVREYREKVETELQGVCDTVLGLLDSHLIKEAGDAESRVFYLKMKGDYYRYLAEVATGDDKKRIIDSARSAYQEAMDISKKEMPPTNPIRLGLALNFSVFHYEIANSPEEAISLAKTTFDEAMADLHTLSEDSYKDSTLIMQLLRDNLTLWTXXXX

SEQ ID NO: 147 - protein, 95 aa, Homo sapiens
  VARIANT 95: note = Any amino acid
PKPGDIFEVELAKNDNSLGISVTGGVNTSVRHGGIYVKAVIPQGAAESDGRIHKGDRVLAVNGVSLEGATHKQAVETLRNTGQVVHLLLEKGQSX

SEQ ID NO: 148 - protein, 352 aa, Homo sapiens
  VARIANT 350: note = Any amino acid
  VARIANT 351: note = Any amino acid
  VARIANT 352: note = Any amino acid
ETLNPSARIMTFYPTMEEFRNFSRYIAYIESQGAHRAGLAKVVPPKEWKPRASYDDIDDLVIPAPIQQLVTGQSGLFTQYNIQKKAMTVREFRKIANSDKYCTPRYSEFEELERKYWKNLTFNPPIYGADVNGTLYEKHVDEWNIGRLRTILDLVEKESGITIEGVNTPYLYFGMWKTSFAWHTEDMDLYSINYLHFGEPKSWYSVPPEHGKRLERLAKGFFPGSAQSCEAFLRHKMTLISPLMLKKYGIPFDKVTQEAGEFMITFPYGYHAGFNHGFNCAESTNFATRRWIEYGKQAVLCSCRKDMVKISMDVFVRKFQPERYKLWKAGKDNTVIDHTLPTPEAAEFLXXX

SEQ ID NO: 149 - protein, 277 aa, Homo sapiens
  VARIANT 275: note = Any amino acid
  VARIANT 276: note = Any amino acid
  VARIANT 277: note = Any amino acid
GKLSLQDVAELIRARACQRVVVMVGAGISTPSGIPDFRSPGSGLYSNLQQYDLPYPEAIFELPFFFHNPKPFFTLAKELYPGNYKPNVTHYFLRLLHDKGLLLRLYTQNIDGLERVSGIPASKLVEAHGTFASATCTVCQRPFPGEDIRADVMADRVPRCPVCTGVVKPDIVFFGEPLPQRFLLHVVDFPMADLLLILGTSLEVEPFASLTEAVRSSVPRLLINRDLVGPLAWHPRSRDVAQLGDVVHGVESLVELLGWTEEMRDLVQRETGKLXXX

SEQ ID NO: 150 - protein, 231 aa, Homo sapiens
  VARIANT 42: note = Any amino acid
  VARIANT 229: note = Any amino acid
  VARIANT 230: note = Any amino acid
  VARIANT 231: note = Any amino acid
AMGSMERASLIQKAKLAEQAERYEDMAAFMKGAVEKGEELSXEERNLLSVAYKNVVGGQRAAWRVLSSIEQKSNKPEVREYREKVETELQGVCDTVLGLLDSHLIKEAGDAESRVFYLKMKGDYYRYLAEVATGDDKKRIIDSARSAYQEAMDISKKEMPPTNPIRLGLALNFSVFHYEIANSPEEAISLAKTTFDEAMADLHTLSEDSYKDSTLIMQLLRDNLTLWTXXX

SEQ ID NO: 151 - protein, 219 aa, Homo sapiens
YVVMTQSPLSLPVTPGEPASISCKSSQSLLDSDGKTYLNWLLQKPGQSPQRLIYLVSKLDSGVPDRFSGSGSGTDFTLKISRVEAEDVGVYYCWQGTHFPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 152 - protein, 65 aa, Homo sapiens
  VARIANT 58: note = Any amino acid
  VARIANT 59: note = Any amino acid
  VARIANT 60: note = Any amino acid
  VARIANT 61: note = Any amino acid
  VARIANT 62: note = Any amino acid
  VARIANT 63: note = Any amino acid
  VARIANT 64: note = Any amino acid
  VARIANT 65: note = Any amino acid
ENMYVNKVWVQCENENCLKWRLLSSEDSAKVDHDEPWYCFMNTDSRYNNCSISEEDFXXXXXXXX

SEQ ID NO: 153 - protein, 231 aa, Homo sapiens
  VARIANT 231: note = Any amino acid
ALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYDHIHRVLDKITDTLIHLMAKAGLTLQQQHQRLAQLLLILSHIRHMSNKGMEHLYSMKCKNVVPLYGLLLEMLDAHRLX

SEQ ID NO: 154 - protein, 280 aa, Homo sapiens
DTLEGSMAQLKKGLESGTVLIQFEQLYRKKPGLAITFAKLPQNLDKNRYKDVLPYDTTRVLLQGNEDYINASYVNMEIPAANLVNKYIATQGPLPHTCAQFWQVVWDQKLSLIVMLTTLTERGRTKCHQYWPDPPDVMNHGGFHIQCQSEDCTIAYVSREMLVTNTQTGEEHTVTHLQYVAWPAHGVPDDSSDFLEFVNYVRSLRVDSEPVLVHSSAGIGRTGVLVTMETAMCLTERNLPIYPLDIVRKMRDQRAMMVQTSSQYKFVCEAILRVYEEGLV

SEQ ID NO: 155 - protein, 430 aa, Homo sapiens
  VARIANT 428: note = Any amino acid
  VARIANT 429: note = Any amino acid
  VARIANT 430: note = Any amino acid
NRCLKANAKSCGECIQAGPNCGWCTNSTFLSARCDDLEALKKKGCPPDDIENPRGSKDIKKNKNVTNKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTTPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECXXX

SEQ ID NO: 156 - protein, 245 aa, Homo sapiens
  VARIANT 244: note = Any amino acid
  VARIANT 245: note = Any amino acid
GVCWIYYPDGGSLVGEVNEDGEMTGEKIAYVYPDERTALYGKFIDGEMIEGKLATLMSTEEGRPHFELMPGNSVYHFDKSTSSCISTNALLPDPYESERVYVAESLISSAGEGLFSKVAVGPNTVMSFYNGVRITHQEVDSRDWALNGATLSLDEETVIDVPEPYNHVSKYCASLGHKANHSFTPNCIYDMFVHPRFGPIKCIRTLRAVEADEELTVAYGYDHSPPEAPEWYQVELKAFQATQXX

SEQ ID NO: 157 - protein, 149 aa, Homo sapiens
SAKDERAREILRGFKLNWMNLRDAETGKILWQGTEDLSVPGVEHEARVPKKILKCKAVSRELNFSSTEQMEKFRLEQKVYFKGQCLEEWFFEFGFVIPNSTNTWQSLIEAAPESQMMPASVLTGNVIIETKFFDDDLLVSTSRVRLFYV

SEQ ID NO: 158 - protein, 152 aa, Homo sapiens
  VARIANT 151: note = Any amino acid
  VARIANT 152: note = Any amino acid
VLDVEVYSSRSKVYVAVDGTTVLEDEAREQGRGIHVIVLNQATGHVMAKRVFDTYSPHEDEAMVLFLNMVAPGRVLICTVKDEGSFHLKDTAKALLRSLGSQAGPALGWRDTWAFVGRKGGPVFGEKHSKSPALSSWGDPVLLKTDVPLSXX

SEQ ID NO: 159 - protein, 103 aa, Homo sapiens
AMGKCPTKVVLLRNMVGAGEVDEDLEVETKEECEKYGKVGKCVIFEIPGAPDDEAVRIFLEFERVESAIKAVVDLNGRYFGGRVVKACFYNLDKFRVLDLAEQ

SEQ ID NO: 160 - protein, 196 aa, Homo sapiens
GLVAEAEAVAAGWMLDFLCLSLCRAFRDGRSEDFRRTRNSAEAIIHGLSSLTACQLRTIYICQFLTRIAAGKTLDAQFENDERITPLESALMIWGSIEKEHDKLHEEIQNLIKIQAIAVCMENGNFKEAEEVFERIFHMPFKSKLLMIISQKDTFHSFFQHFSYNHMMEKIKSYVNYVLSEKSSTFLMKAAAKVVE

SEQ ID NO: 161 - protein, 191 aa, Homo sapiens
QDKIEALSSKVQQLERSIGLKDLAMADLEQKVLEMEASTYDGVFIWKISDFARKRQEAVAGRIPAIFSPAFYTSRYGYKMCLRIYLNGDGTGRGTHLSLFFVVMKGPNDALLRWPFNQKVTLMLLDQNNREHVIDAFRPDVTSSSFQRPVNDMNIASGCPLFCPVSKMEAKNSYVRDDAIFIKAIVDLTGL

SEQ ID NO: 162 - protein, 232 aa, Homo sapiens
MDKNELVQKAKLAEQAERYDDMAACMKSVTEQGAELSNEERNLLSVAYKNVVGARRSSWRVVSSIEQKTEGAEKKQQMAREYREKIETELRDICNDVLSLLEKFLIPNASQAESKVFYLKMKGDYYRYLAEVAAGDDKKGIVDQSQQAYQEAFEISKKEMQPTHPIRLGLALNFSVFYYEILNSPEKACSLAKTAFDEAIAELDTLSEESYKDSTLIMQLLRDNLTLWTSDT

SEQ ID NO: 163 - protein, 148 aa, Homo sapiens
  VARIANT 146: note = Any amino acid
  VARIANT 147: note = Any amino acid
  VARIANT 148: note = Any amino acid
EPAMEPETLEARINRATNPLNKELDWASINGFCEQLNEDFEGPPLATRLLAHKIQSPQEWEAIQALTVLETCMKSCGKRFHDEVGKFRFLNELIKVVSPKYLGSRTSEKVKNKILELLYSWTVGLPEEVKIAEAYQMLKKQGIVKXXX

SEQ ID NO: 164 - protein, 304 aa, Homo sapiens
PGTVDKKMVEKCWKLMDKVVRLCQNPKLALKNSPPYILDLLPDTYQHLRTILSRYEGKMETLGENEYFRVFMENLMKKTKQTISLFKEGKERMYEENSQPRRNLTKLSLIFSHMLAELKGIFPSGLFQGDTFRITKADAAEFWRKAFGEKTIVPWKSFRQALHEVHPISSGLEAMALKSTIDLTCNDYISVFEFDIFTRLFQPWSSLLRNWNSLAVTHPGYMAFLTYDEVKARLQKFIHKPGSYIFRLSCTRLGQWAIGYVTADGNILQTIPHNKPLFQALIDGFREGFYLFPDGRNQNPDLTG

SEQ ID NO: 165 - protein, 217 aa, Homo sapiens
  VARIANT 216: note = Any amino acid
  VARIANT 217: note = Any amino acid
LPALKLALEYIVPCMNKHGICVVDDFLGKETGQQIGDEVRALHDTGKFTDGQLVSQKSDSSKDIRGDKITWIEGKEPGCETIGLLMSSMDDLIRHCNGKLGSYKINGRTKAMVACYPGNGTGYVRHVDNPNGDGRCVTCIYYLNKDWDAKVSGGILRIFPEGKAQFADIEPKFDRLLFFWSDRRNPHEVQPAYATRYAITVWYFDADERARAKVKXX

SEQ ID NO: 166 - protein, 130 aa, Homo sapiens
  VARIANT 128: note = Any amino acid
  VARIANT 129: note = Any amino acid
  VARIANT 130: note = Any amino acid
GPLGSMGIVSCTACGQQVNHFQKDSIYRHPSLQVLICKNCFKYYMSDDISRDSDGMDEQCRWCAEGGNLICCDFCHNAFCKKCILRNLGRRELSTIMDENNQWYCYICHPEPLLDLVTACNSVYENLXXX

SEQ ID NO: 167 - protein, 71 aa, Homo sapiens
  VARIANT 68: note = Any amino acid
  VARIANT 69: note = Any amino acid
  VARIANT 70: note = Any amino acid
  VARIANT 71: note = Any amino acid
SGPSCKHCKDDVNRLCRVCACHLCGGRQDPDKQLMCDECDMAFHIYCLDPPLSSVPSEDEWYCPECRXXXX

SEQ ID NO: 168 - protein, 310 aa, Homo sapiens
  VARIANT 307: note = Any amino acid
  VARIANT 308: note = Any amino acid
  VARIANT 309: note = Any amino acid
  VARIANT 310: note = Any amino acid
APLLLYANRRDLRLVDATNGKENATIVVGGLEDAAAVDFVFSHGLIYWSDVSEEAIKRTEFNKTESVQNVVVSGLLSPDGLACDWLGEKLYWTDSETNRIEVSNLDGSLRKVLFWQELDQPRAIALDPSSGFMYWTDWGEVPKIERAGMDGSSRFIIINSEIYWPNGLTLDYEEQKLYWADAKLNFIHKSNLDGTNRQAVVKGSLPHPFALTLFEDILYWTDWSTHSILACNKYTGEGLREIHSDIFSPMDIHAFSQQRQPNATNPCGIDNGGCSHLCLMSPVKPFYQCACPTGVKLLENGKTCKDXXXX

SEQ ID NO: 169 - protein, 116 aa, Homo sapiens
  VARIANT 113: note = Any amino acid
  VARIANT 114: note = Any amino acid
  VARIANT 115: note = Any amino acid
  VARIANT 116: note = Any amino acid
HMEPIPICSFCLGTKEQNREKKPEELISCADCGNSGHPSCLKFSPELTVRVKALRWQCIECKTCSSCRDQGKNADNMLFCDSCDRGFHMECCDPPLTRMPKGMWICQICRPRXXXX

SEQ ID NO: 170 - protein, 304 aa, Homo sapiens
VKPNYALKFTLAGHTKAVSSVKFSPNGEWLASSSADKLIKIWGAYDGKFEKTISGHKLGISDVAWSSDSNLLVSASDDKTLKIWDVSSGKCLKTLKGHSNYVFCCNFNPQSNLIVSGSFDESVRIWDVKTGKCLKTLPAHSDPVSAVHFNRDGSLIVSSSYDGLCRIWDTASGQCLKTLIDDDNPPVSFVKFSPNGKYILAATLDNTLKLWDYSKGKCLKTYTGHKNEKYCIFANFSVTGGKWIVSGSEDNLVYIWNLQTKEIVQKLQGHTDVVISTACHPTENIIASAALENDKTIKLWKSDC

SEQ ID NO: 171 - protein, 134 aa, Homo sapiens
THMVSLPEELNRVRLSRHKLERWCHMPFFAKTVTGCFVRIGIGNHNSKPVYRVAEITGVVETAKVYQLGGTRTNKGLQLRHGNDQRVFRLEFVSNQEFTESEFMKWKEAMFSAGMQLPTLDEINKKELSIKEAL

SEQ ID NO: 172 - protein, 60 aa, Homo sapiens
  VARIANT 59: note = Any amino acid
  VARIANT 60: note = Any amino acid
GPLPNALYCICRQPHNNRFMICCDRCEEWFHGDCVGISEARGRLLERNGEDYICPNCTXX

SEQ ID NO: 173 - protein, 171 aa, Homo sapiens
LIPAPPLSKVPLQQNFQDNQFHGKWYVVGVAGNKSLREDKDPWKMYATIYELKEDKSYNVTSVGFGTKKCHYKIRTFVPGSQPGEFTLGRIKSRPGRTSALVRVVSTNYNQHAMVFFKVVQQNRESFNITLYGRTKELTSELKENFIRFSKSLGLPENHIVFPVPIDQCID

SEQ ID NO: 174 - protein, 203 aa, Homo sapiens
LSCPEGLEELLSAPPPDLGAQRRHGWNPKDCSENIEVKEGGLYFERRPVAQSTDGARGKRGYSRGLHAWEISWPLEQRGTHAVVGVATALAPLQTDHYAALLGSNSESWGWDIGRGKLYHQSKGPGAPQYPAGTQGEQLEVPERLLVVLDMEEGTLGYAIGGTYLGPAFRGLKGRTLYPAVSAVWGQCQVRIRYLGERGSHHH

SEQ ID NO: 175 - protein, 249 aa, Homo sapiens
  VARIANT 247: note = Any amino acid
  VARIANT 248: note = Any amino acid
  VARIANT 249: note = Any amino acid
IIGGEFTTIENQPWFAAIYRRHRGGSVTYVCGGSLMSPCWVISATHCFIDYPKKEDYIVYLGRSRLNSNTQGEMKFEVENLILHKDYSADTLAHHNDIALLKIRSKEGRCAQPSRTIQTICLPSMYNDPQFGTSCEITGFGKEASTDYLYPEQLKMTVVKLISHRECQQPHYYGSEVTTKMLCAADPQWKTDSCQGDSGGPLVCSLQGRMTLTGIVSWGRGCALKDKPGVYTRVSHFLPWIRSHTKXXX

SEQ ID NO: 176 - protein, 236 aa, Homo sapiens
  VARIANT 77: note = Any amino acid
  VARIANT 113: note = Any amino acid
  VARIANT 217: note = Any amino acid
  VARIANT 236: note = Any amino acid
SLALSLTADQMVSALLDAEPPILYSEYDPTRPFSEASMMGLLTNLADRELVHMINWAKRVPGFVDLTLHDQVHLLEXAWLEILMIGLVWRSMEHPGKLLFAPNLLLDRNQGKXVEGMVEIFDMLLATSSRFRMMNLQGEEFVCLKSIILLNSGVYTFEKDHIHRVLDKITDTLIHLMAKAGLTLQQQHQRLAQLLLILSHIRHMSNKGMEHLYSMKXKNVVPLSDLLLEMLDAHRX

SEQ ID NO: 177 - protein, 92 aa, Homo sapiens
KEKKVFISLVGSRGLGCSISSGPIQKPGIFISHVKPGSLSAEVGLEIGDQIVEVNGVDFSNLDHKEAVNVLKSSRSLTISIVAAAGRELFMT

SEQ ID NO: 178 - protein, 169 aa, Homo sapiens
VDMVQLLKKYPIVWQGLLALKNDTAAVQLHFVSGNNVLAHRSLPLSEGGPPLRIAQRMRLEATQLEGVARRMTVETDYCLLLALPCGRDQEDVVSQTESLKAAFITYLQAKQAAGIINVPNPGSNQPAYVLQIFPPCEFSESHLSRLAPDLLASISNISPHLMIVIASV

SEQ ID NO: 179 - protein, 102 aa, Homo sapiens
  VARIANT 101: note = Any amino acid
  VARIANT 102: note = Any amino acid
GRLWLESPPGEAPPIFLPSDGQALVLGRGPLTQVTDRKCSRTQVELVADPETRTVAVKQLGVNPSTTGTQELKPGLEGSLGVGDTLYLVNGEHPLTLRWEXX

SEQ ID NO: 180 - protein, 266 aa, Homo sapiens
  VARIANT 266: note = Any amino acid
DLRALAKHLYDSYIKSFPLTKAKARAILTGKTTDKSPFVIYDMNSLMMGEDKIKFKHIQEQSKEVAIRIFQGCQFRSVEAVQEITEYAKSIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIYKDLYX

SEQ ID NO: 181 - protein, 424 aa, Homo sapiens
  VARIANT 423: note = Any amino acid
  VARIANT 424: note = Any amino acid
SALNFDSPSSLFESLISPIKTETFFKEFWEQKPLLIQRDDPALATYYGSLFKLTDLKSLCSRGMYYGRDVNVCRCVNGKKKVLNKDGKAHFLQLRKDFDQKRATIQFHQPQRFKDELWRIQEKLECYFGSLVGSNVYITPAGSQGLPPHYDDVEVFILQLEGEKHWRLYHPTVPLARECSVEAEERIGRPVHEFMLKPGDLLYFPRGTIHQADTPAGLAHSTHVTISTYQNNSWGDFLLDTISGLVFDTAKEDVELRTGIPRQLLLQVESTTVATRRLSGFLRTLADRLEGTKELLSSDMKKDFIMHRLPPYSAGDGAELSTPGGKLPRLDSVVRLQFKDHIVLTVLPQEKMVYIYHSLKNSRETHMMTEFHGLRFPLSHLDALKQIWNSPAISVKDLKLTTDEEKESLVLSLWTECLIQVVXX

SEQ ID NO: 182 - protein, 114 aa, Homo sapiens
MPSQMEHAMETMMFTFHKFAGDKGYLTKEDLRVLMEKEFPGFLENQKDPLAVDKIMKDLDQCRDGKVGFQSFFSLIAGLTIACNDYFVVHMKQENLYFQGDSTVHEILSKLSLE

SEQ ID NO: 183 - protein, 332 aa, Homo sapiens
  VARIANT 330: note = Any amino acid
  VARIANT 331: note = Any amino acid
  VARIANT 332: note = Any amino acid
QNPSCKIMTFRPTMEEFKDFNKYVAYIESQGAHRAGLAKIIPPKEWKPRQTYDDIDDVVIPAPIQQVVTGQSGLFTQYNIQKKAMTVGEYRRLANSEKYCTPRHQDFDDLERKYWKNLTFVSPIYGADISGSLYDDDVAQWNIGSLRTILDMVERECGTIIEGVNTPYLYFGMWKTTFAWHTEDMDLYSINYLHFGEPKSWYAIPPEHGKRLERLAIGFFPGSSQGCDAFLRHKMTLISPIILKKYGIPFSRITQEAGEFMITFPYGYHAGFNHGFNCAESTNFATLRWIDYGKVATQCTCRKDMVKISMDVFVRILQPERYELWKQGKXXX

SEQ ID NO: 184 - protein, 246 aa, Homo sapiens
  VARIANT 246: note = Any amino acid
PYASLTEIEHLVQSVCKSYRETCQLRLEDLLRQRSNIFSREEVTGYQRKSMWEMWERCAHHLTEAIQYVVEFAKRLSGFMELCQNDQIVLLKAGAMEVVLVRMCRAYNADNRTVFFEGKYGGMELFRALGCSELISSIFDFSHSLSALHFSEDEIALYTALVLINAHRPGLQEKRKVEQLQYNLELAFHHHLCKTHRQSILAKLPPKGKLRSLCSQHVERLQIFQHLHPIVVQAAFPPLYKELFSX

SEQ ID NO: 185 - protein, 77 aa, Homo sapiens
  VARIANT 27: note = Any amino acid
EYIKLKVIGQDSSEIHFKVKMTTHLKXLKESYAQRQGVPMNSLRFLFEGQRIADNHTPKELGMEEEDVIEVYQEQTG

SEQ ID NO: 186 - protein, 244 aa, Homo sapiens
  VARIANT 244: note = Any amino acid
SKKNSLALSLTADQMVSALLDAEPPILYSEPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRX

SEQ ID NO: 187 - protein, 164 aa, Homo sapiens
DNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQT

SEQ ID NO: 188 - protein, 241 aa, Homo sapiens
GITQSPKYLFRKEGQNVTLSCEQNLNHDAMYWYRQDPGQGLRLIYYSQIVNDFQKGDIAEGYSVSREKKESFPLTVTSAQKNPTAFYLCASSSRSSYEQYFGPGTRLTVTEDLKNVFPPEVAVFEPSEAEISHTQKATLVCLATGFYPDHVELSWWVNGKEVHSGVSTDPQPLKEQPALNDSRYSLSSRLRVSATFWQNPRNHFRCQVQFYGLSENDEWTQDRAKPVTQIVSAEAWGRADQ

SEQ ID NO: 189 - protein, 275 aa, Homo sapiens [sequence omitted]

SEQ ID NO: 190 - protein, 277 aa, Homo sapiens
  VARIANT 277: note = Any amino acid
GSHSMRYFYTAMSRPGRGEPRFIAVGYVDDTQFVRFDSDAASPRMAPRAPWIEQEGPEYWDRETQISKTNTQTYRESLRNLRGYYNQSEAGSHTLQRMYGCDVGPDGRLLRGHDQSAYDGKDYIALNEDLSSWTAADTAAQITQRKWEAAREAEQWRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPX

SEQ ID NO: 191 - protein, 306 aa, Homo sapiens
  VARIANT 306: note = Any amino acid
PPGTVDKKMVEKCWKLMDKVVRLCQNPKLALKNSPPYILDLLPDTYQHLRTILSRYEGKMETLGENEYFRVFMENLMKKTKQTISLFKEGKERMYEENSQPRRNLTKLSLIFSHMLAELKGIFPSGLFQGDTFRITKADAAEFWRKAFGEKTIVPWKSFRQALHEVHPISSGLEAMALKSTIDLTCNDYISVFEFDIFTRLFQPWSSLLRNWNSLAVTHPGYMAFLTYDEVKARLQKFIHKPGSYIFRLSCTRLGQWAIGYVTADGNILQTIPHNKPLFQALIDGFREGFYLFPDGRNQNPDLTGX

SEQ ID NO: 192 - protein, 60 aa, Homo sapiens
NDLGITAVALYDYQAAGDDEISFDPDDIITNIEMIDDGWWRGVCKGRYGLFPANYVELRQ

SEQ ID NO: 193 - protein, 380 aa, Homo sapiens
  VARIANT 378: note = Any amino acid
  VARIANT 379: note = Any amino acid
  VARIANT 380: note = Any amino acid
DKKMVEKCWKLMDKVVRLCQNPKLALKNSPPYILDLLPDTYQHLRTILSRYEGKMETLGENEYFRVFMENLMKKTKQTISLFKEGKERMYEENSQPRRNLTKLSLIFSHMLAELKGIFPSGLFQGDTFRITKADAAEFWRKAFGEKTIVPWKSFRQALHEVHPISSGLEAMALKSTIDLTCNDYISVFEFDIFTRLFQPWSSLLRNWNSLAVTHPGYMAFLTYDEVKARLQKFIHKPGSYIFRLSCTRLGQWAIGYVTADGNILQTIPHNKPLFQALIDGFREGFYLFPDGRNQNPDLTGLCEKVTQEQYELYCEMGSTFQLCKICAENDKDVKIEPCGHLMCTSCLTSWQESEGQGCPFCRCEIKGTEPIVVDPFDXXX

SEQ ID NO: 194 - protein, 218 aa, Homo sapiens
KQYIFQLSSLNPQERIDYCHLIEKLGGLVIEKQCFDPTCTHIVVGHPLRNEKYLASVAAGKWVLHRSYLEACRTAGHFVQEEDYEWGSSSILDVLTGINVQQRRLALAAMRWRKKIQQRQESGIVEGAFSGWKVILHVDQSREAGFKRLLQSGGAKVLPGHSVPLFKEATHLFSDLGVNIAEAAAQNVYCLRTEYIADYLMQESPPHVENYCLPEAIS

SEQ ID NO: 195 - protein, 240 aa, Homo sapiens
  VARIANT 76: note = Any amino acid
  VARIANT 112: note = Any amino acid
  VARIANT 240: note = Any amino acid
LALSLTADQMVSALLDAEPPILYSEYDPTRPFSEASMMGLLTNLADRELVHMINWAKRVPGFVDLTLHDQVHLLEXAWLEILMIGLVWRSMEHPGKLLFAPNLLLDRNQGKXVEGMVEIFDMLLATSSRFRMMNLQGEEFVCLKSIILLNSGVYTFKSLEEKDHIHRVLDKITDTLIHLMAKAGLTLQQQHQRLAQLLLILSHIRHMSNKGMEHLYSMKCKNVVPLSDLLLEMLDAHRLX

SEQ ID NO: 196 - protein, 230 aa, Homo sapiens
  VARIANT 230: note = Any amino acid
VDREQLVQKARLAEQAERYDDMAAAMKNVTELNEPLSNEERNLLSVAYKNVVGARRSSWRVISSIEQKTSNEKIEMVRAYREKIEKELEAVCQDVLSLLDNYLIKNCSETQYESKVFYLKMKGDYYRYLAEVATGEKRATVVESSEKAYSEAHEISKEHMQPTHPIRLGLALNYSVFYYEIQNAPEQACHLAKTAFDDAIAELDTLNEDSYKDSTLIMQLLRDNLTLWTX

SEQ ID NO: 197 - protein, 287 aa, Homo sapiens
  VARIANT 287: note = Any amino acid
PKVMIVVGGQAPKAIRSVECYDFEEDRWDQIAELPSRRCRAGVVFMAGHVYAVGGFNGSLRVRTVDVYDGVKDQWTSIASMQERRSTLGAAVLNDLLYAVGGFDGSTGLASVEAYSYKTNEWFFVAPMNTRRSSVGVGVVEGKLYAVGGYDGASRQCLSTVEQYNPATNEWIYVADMSTRRSGAGVGVLSGQLYATGGHDGPLVRKSVEVYDPGTNTWKQVADMNMCRRNAGVCAVNGLLYVVGGDDGSCNLASVEYYNPVTDKWTLLPTNMSTGRSYAGVAVIHKX

SEQ ID NO: 198 - protein, 240 aa, Homo sapiens
  VARIANT 240: note = Any amino acid
SYEMTAELDDLTEKIRKAHQETFPSLCQLGKYTTNSSADHRVRLDLGLWDKFSELATKCIIKIVEFAKRLPGFTGLTIADQITLLKAACLDILILRICTRYTPEQDTMTFSDGLTLNRTQMHNAGFGPLTDLVFTFANQLLPLEMDDTETGLLSAICLICGDRQDLEEPTKVDKLQEPLLEALKIYIRKRRPSKPHMFPKILMKITDLRSISAKGAERVITLKMEIPGSMPPLIQEMMEX

SEQ ID NO: 199 - protein, 277 aa, Homo sapiens
  VARIANT 275: note = Any amino acid
  VARIANT 276: note = Any amino acid
  VARIANT 277: note = Any amino acid
GSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWXXX

SEQ ID NO: 200 - protein, 110 aa, Homo sapiens
GPLGSMSGGFELQPRDGGPRVALAPGETVIGRGPLLGITDKRVSRRHAILEVAGGQLRIKPIHTNPCFYQSSEKSQLLPLKPNLWCYLNPGDSFSMLVDKYIFRILSIPS

SEQ ID NO: 201 - protein, 228 aa, Homo sapiens
  VARIANT 228: note = Any amino acid
SLTEIEHLVQSVCKSYRETCQLRLEDLLRQRSNIFSREEVTGYQRKSMWEMWERCAHHLTEAIQYVVEFAKRLSGFMELCQNDQIVLLKAGAMEVVLVRMCRAYNADNRTVFFEGKYGGMELFRALGCSELISSIFDFSHSLSALHFSEDEIALYTALVLINAHRPGLQEKRKVEQLQYNLELAFHHHLCKTHRQSILAKLPPAGKLASLCSQHVERLQIFQHLHPIX

SEQ ID NO: 202 - protein, 120 aa, Homo sapiens
GSPASGTSLSAAIHRTQLWFHGRISREESQRLIGQQGLVDGLFLVRESQRNPQGFVLSLCHLQKVKHYLILPSEEEGRLYFSMDDGQTRFTDLLQLVEFHQLNRGILPCLLRHCCTRVAL

SEQ ID NO: 203 - protein, 142 aa, Homo sapiens
ASITVPLESIKPSNILPVTVYDQHGFRILFHFARDPLPGRSDVLVVVVSMLSTAPQPIRNIVFQSAVPKVMKVKLQPPSGTELPAFNPIVHPSAITQVLLLANPQKEKVRLRYKLTFTMGDQTYNEMGDVDQFPPPETWGSL

SEQ ID NO: 204 - protein, 67 aa, Homo sapiens [sequence omitted]

SEQ ID NO: 205 - protein, 242 aa, Homo sapiens
  VARIANT 242: note = Any amino acid
PIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIISCSRRFYQLTKLLDSVQPIARELHQFTFDLLIKSHMVSVDFPEMMAEIISVQVPKILSGKVKPIYFHTQX

SEQ ID NO: 206 - protein, 240 aa, Homo sapiens
  VARIANT 240: note = Any amino acid
IIGGSSSLPGSHPWLAAIYIGDSFCAGSLVHTCWVVSAAHCFSHSPPRDSVSVVLGQHFFNRTTDVTQTFGIEKYIPYTLYSVFNPSDHDLVLIRLKKKGDRCATRSQFVQPICLPEPGSTFPAGHKCQIAGWGHLDENVSGYSSSLREALVPLVADHKCSSPEVYGADISPNMLCAGYFDCKSDACQGDSGGPLACEKNGVAYLYGIISWGDGCGRLHKPGVYTRVANYVDWINDRIRX

SEQ ID NO: 207 - protein, 70 aa, Homo sapiens
  VARIANT 70: note = Any amino acid
GSSGSSGEISGFGQCLVWVQCSFPNCGKWRRLCGNIDPSVLPDNWSCDQNTDVQYNRCDIPEETWTGLEX

SEQ ID NO: 208 - protein, 236 aa, Homo sapiens
  VARIANT 225: note = Any amino acid
  VARIANT 236: note = Any amino acid
ALSPEQLVLTLLEAEPPHVLISRPSAPFTEASMMMSLTKLADKELVHMISWAKKIPGFVELSLFDQVRLLESCWMEVLMMGLMWRSIDHPGKLIFAPDLVLDRDEGKCVEGILEIFDMLLATTSRFRELKLQHKEYLCVKAMILLNSSMYPLVTATADSSRKLAHLLNAVTDALVWVIAKSGISSQQQSMRLANLLMLLSHVRHASNKGMEHLLNMKCKNVVPVXDLLLEMLNAHX

SEQ ID NO: 209 - protein, 305 aa, Homo sapiens
  VARIANT 303: note = Any amino acid
  VARIANT 304: note = Any amino acid
  VARIANT 305: note = Any amino acid
RTVEKTWKLMDKVVRLCQNPKLQLKNSPPYILDILPDTYQHLRLILSKYDDNQKLAQLSENEYFKIYIDSLMKKSKRAIRLFKEGKERMYEEQSQDRRNLTKLSLIFSHMLAEIKAIFPNGQFQGDNFRITKADAAEFWRKFFGDKTIVPWKVFRQCLHEVHQISSGLEAMALKSTIDLTCNDYISVFEFDIFTRLFQPWGSILRNWNFLAVTHPGYMAFLTYDEVKARLQKYSTKPGSYIFRLSCTRLGQWAIGYVTGDGNILQTIPHNKPLFQALIDGSREGFYLYPDGRSYNPDLTGLAXXX

SEQ ID NO: 210 - protein, 323 aa, Homo sapiens
RREIRVLHLLEQIRAYCETCWEWQEAHNPMPAPVEHQICPAVCVLMKLSFDEEHRHAMNELGGLQAIAELLQVDCEMYGLTNDHYSITLRRYAGMALTNLTFGDVANKATLCSMKGCMRALVAQLKSESEDLQQVIASVLRNLSWRADVNSKKTLREVGSVKALMECALEVKKESTLKSVLSALWNLSAHCTENKADICAVDGALAFLVGTLTYRSQTNTLAIIESGGGILRNVSSLIATNEDHRQILRENNCLQTLLQHLKSHSLTIVSNACGTLWNLSARNPKDQEALWDMGAVSMLKNLIHSKHKMIAMGSAAALRNLMA

SEQ ID NO: 211 - protein, 85 aa, Homo sapiens
KAVIKNADMSEEMQQDSVECATQALEKYNIEKDIAAHIKKEFDKKYNPTWHCIVGRNFGSYVTHETKHFIYFYLGQVAILLFKSG

SEQ ID NO: 212 - protein, 221 aa, Homo sapiens
SQTVASHVPFADLCSTLERIQKSKGRAEKIRHFREFLDSWRKFHDALHKNHKDVTDSFYPAMRLILPQLERERMAYGIKETMLAKLYIELLNLPRDGKDALKLLNYGDFAMIAYFVLKPRCLQKGSLTIQQVNDLLDSIASNNSAKRKDLIKKSLLQLITQSSALEQKWLIRMIIKDLKLGVSQQTIFSVFHNDAAELHNVTTDLEKVCRQLHDPSVGLSD

SEQ ID NO: 213 - protein, 368 aa, Homo sapiens
  VARIANT 368: note = Any amino acid
SLFPPGLHAIYGECRRLYPDQPNPLQVTAIVKYWLGGPDPLDYVSMYRNVGSPSANIPEHWHYISFGLSDLYGDNRVHEFTGTDGPSGFGFELTFRLKRETGESAPPTWPAELMQGLARYVFQSENTFCSGDHVSWHSPLDNSESRIQHMLLTEDPQMQPVQTPFGVVTFLQIVGVCTEELHSAQQWNGQGILELLRTVPIAGGPWLITDMRRGETIFEIDPHLQERVDKGIETDGSNLSGVSAKCAWDDLELIRTRQLESVHLKFNQESGALIPLCLRGRLLHGRHFTYKSITGDMAITFVSTGVEGAFATEEHPYAAHGPWLQILLTEEFVEKMLEDLEDLKLPKEYSWPEKKLKVSILPDVVFDX

SEQ ID NO: 214 - protein, 698 aa, Homo sapiens
  VARIANT 698: note = Any amino acid
CPTHADSLNNLANIKREQGNIEEAVRLYRKALEVFPEFAAAHSNLASVLQQQGKLQEALMHYKEAIRISPTFADAYSNMGNTLKEMQDVQGALQCYTRAIQINPAFADAHSNLASIHKDSGNIPEAIASYRTALKLKPDFPDAYCNLAHCLQIVCDWTDYDERMKKLVSIVADQLEKNRLPSVHPHHSMLYPLSHGFRKAIAERHGNLCLDKINVLHKPPYEHPKDLKLSDGRLRVGYVSSDFGNHPTSHLMQSIPGMHNPDKFEVFCYALSPDDGTNFRVKVMAEANHFIDLSQIPCNGKAADRIHQDGIHILVNMNGYTKGARNELFALRPAPIQAMWLGYPGTSGALFMDYIITDQETSPAEVAEQYSEKLAYMPHTFFIGDHANMFPHLKKKAVIDFKIYDNRIVLNGIDLKAFLDSLPDVKIVKMLNMPVIPMNTIAEAVIEMINRGQIQITINGFSISNGLATTQINNKAATGEEVPRTIIVTTRSQYGLPEDAIVYCNFNQLYKIDPSTLQMWANILKRVPNSVLWLLRFPAVGEPNIQQYAQNMGLPQNRIIFSPVAPKEEHVRRGQLADVCLDTPLCNGHTTGMDVLWAGTPMVTMPGETLASRVAASQLTCLGCLELIAKNRQEYEDIAVKLGTDLEYLKKVRGKVWKQRISSPLFNTKQYTMELERLYLQMWEHYAAGNKPDHMIKX

SEQ ID NO: 215 - protein, 258 aa, Homo sapiens
  VARIANT 258: note = Any amino acid
SADLRALAKHLYDSYIKSFPLTKAKARAILTGKTTDKSPFVIYDMNSLMMGEDKIKEVAIRIFQGCQFRSVEAVQEITEYAKSIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIYKDLX

SEQ ID NO: 216 - protein, 336 aa, Homo sapiens
HHHMLHLLEQIRAYCETCWEWQEAHEPGMDQDKNPMPAPVEHQICPAVCVLMKLSFDEEHRHAMNELGGLQAIAELLQVDCEMYGLTNDHYSITLRRYAGMALTNLTFGDVANKATLCSMKGCMRALVAQLKSESEDLQQVIASVLRNLSWRADVNSKKTLREVGSVKALMECALEVKKESTLKSVLSALWNLSAHCTENKADICAVDGALAFLVGTLTYRSQTNTLAIIESGGGILRNVSSLIATNEDHRQILRENNCLQTLLQHLKSHSLTIVSNACGTLWNLSARNPKDQEALWDMGAVSMLKNLIHSKHKMIAMGSAAALRNLMANRPAKYK

SEQ ID NO: 217 - protein, 169 aa, Homo sapiens
NRLIVDEAINEDNSVVSLSQPKMDELQLFRGDTVLLKGKKRREAVCIVLSDDTCSDEKIRMNRVVRNNLRVRLGDVISIQPCPDVKYGKRIHVLPIDDTVEGITGNLFEVYLKPYFLEAYRPIRKGDIFLVRGGMRAVEFKVVETDPSPYCIVAPDTVIHCEGEPIKRE

SEQ ID NO: 218 - protein, 428 aa, Homo sapiens
NQGTVNWSVDDIVKGINSSNVENQLQATQAARKLLSREKQPPIDNIIRAGLIPKFVSFLGRTDCSPIQFESAWALTNIASGTSEQTKAVVDGGAIPAFISLLASPHAHISEQAVWALGNIAGDGSVFRDLVIKYGAVDPLLALLAVPDMSSLACGYLRNLTWTLSNLCRNKNPAPPIDAVEQILPTLVRLLHHDDPEVLADTCWAISYLTDGPNERIGMVVKTGVVPQLVKLLGASELPIVTPALRAIGNIVTGTDEQTQVVIDAGALAVFPSLLTNPKTNIQKEATWTMSNITAGRQDQIQQVVNHGLVPFLVSVLSKADFKTQKEAVWAVTNYTSGGTVEQIVYLVHCGIIEPLMNLLTAKDTKIILVILDAISNIFQAAEKLGETEKLSIMIEECGGLDKIEALQNHENESVYKASLSLIEKYFS

SEQ ID NO: 219 - protein, 254 aa, Homo sapiens
  VARIANT 253: note = Any amino acid
  VARIANT 254: note = Any amino acid
ENLYFQGASLTEIEHLVQSVCKSYRETCQLRLEDLLRQRSNIFSREEVTGYQRKSMWEMWERCAHHLTEAIQYVVEFAKRLSGFMELCQNDQIVLLKAGAMEVVLVRMCRAYNADNRTVFFEGKYGGMELFRALGCSELISSIFDFSHSLSALHFSEDEIALYTALVLINAHRPGLQEKRKVEQLQYNLELAFHHHLCKTHRQSILAKLPPKGKLRSLCSQHVERLQIFQHLHPIVVQAAFPPLYKELFSGGXX

SEQ ID NO: 220 - protein, 228 aa, Homo sapiens
  VARIANT 228: note = Any amino acid
LTPDQQTLLHFIMDSYNKQRMPQEITNKILKEEFSAEENFLILTEMATNHVQVLVEFTKKLPGFQTLDHEDQIALLKGSAVEAMFLRSAEIFNKKLPSGHSDLLEERIRNSGISDEYITPMFSFYKSIGELKMTQEEYALLTAIVILSPDRQYIKDREAVEKLQEPLLDVLQKLCKIHQPENPQHFACLLGRLTELRTFNHHHAEMLMSWRVNDHKFTPLLCEIWDVX

SEQ ID NO: 221 - protein, 62 aa, Homo sapiens [sequence omitted]

SEQ ID NO: 222 - protein, 62 aa, Homo sapiens
  VARIANT 61: note = Any amino acid
  VARIANT 62: note = Any amino acid
LIKHMRAEALFDFTGNSKLELNFKAGDVIFLLSRINKDWLEGTVRGATGIFPLSFVKILKXX

SEQ ID NO: 223 - protein, 81 aa, Homo sapiens
DSVIVVDNVPQVGPDRLEKLKNVIHKIFSKFGKITNDFYPEEDGKTKGYIFLEYASPAHAVDAVKNADGYKLDKQHTFRVN

SEQ ID NO: 224 - protein, 363 aa, Homo sapiens
DNVLPVDSDLSPNISTNTSEPNQFVQPAWQIVLWAAAYTVIVVTSVVGNVVVMWIILAHKRMRTVTNYFLVNLAFAEASMAAFNTVVNFTYAVHNEWYYGLFYCKFHNFFPIAAVFASIYSMTAVAFDRYMAIIHPLQPRLSATATKVVICVIWVLALLLAFPQGYYSTTETMPSRVVCMIEWPEHPNKIYEKVYHICVTVLIYFLPLLVIGYAYTVVGITLWASEIPGDSSDRYHEQVSAKRKVVKMMIVVVCTFAICWLPFHIFFLLPYINPDLYLKKFIQQVYLAIMWLAMSSTMYNPIIYCCLNDRFRLGFKHAFRCCPFISAGDYEGLEMKSTRYLQTQGSVYKVSRLETTISTVVGA

SEQ ID NO: 225 - protein, 250 aa, Homo sapiens
  VARIANT 250: note = Any amino acid
APTLISLLEVIEPEVLYSGYDSTLPDTSTRLMSTLNRLGGRQVVSAVKWAKALPGFRNLHLDDQMTLLQYSWMSLMAFSLGWRSYKQSNGNMLCFAPDLVINEERMQLPYMYDQCQQMLKISSEFVRLQVSYDEYLCMKVLLLLSTVPKDGLKSQAVFDEIRMTYIKELGKAIVKREGNSSQNWQRFYQLTKLLDSMHEMVGGLLQFCFYTFVNKSLSVEFPEMLAEIISNQLPKFKAGSVKPLLFHQKX

SEQ ID NO: 226 - protein, 359 aa, Homo sapiens
  VARIANT 358: note = Any amino acid
  VARIANT 359: note = Any amino acid
TDRVVKAVPFPPSHRLTAKEVFDNDGKPRVDILKAHLMKEGRLEESVALRIITEGASILRQEKNLLDIDAPVTVCGDIHGQFFDLMKLFEVGGSPANTRYLFLGDYVDRGYFSIECVLYLWALKILYPKTLFLLRGNHECRHLTEYFTFKQECKIKYSERVYDACMDAFDCLPLAALMNQQFLCVHGGLSPEINTLDDIRKLDRFKEPPAYGPMCDILWSDPLEDFGNEKTQEHFTHNTVRGCSYFYSYPAVCEFLQHNNLLSILRAHEAQDAGYRMYRKSQTTGFPSLITIFSAPNYLDVYNNKAAVLKYENNVMNIRQFNCSPHPYWLPNFMDVFTWSLPFVGEKVTEMLVNVLNXX

SEQ ID NO: 227 - protein, 79 aa, Homo sapiens
GEYIKLKVIGQDSSEIHFKVKMTTHLKKLKESYAQRQGVPMNSLRFLFEGQRIADNHTPKELGMEEEDVIEVYQEQTGG

SEQ ID NO: 228 - protein, 339 aa, Homo sapiens
HHHMLHLLEQIRAYCETCWEWQEAHEPGMDQDKNPMPAPVEHQICPAVCVLMKLSFDEEHRHAMNELGGLQAIAELLQVDCEMYGLTNDHYSITLRRYAGMALTNLTFGDVANKATLCSMKGCMRALVAQLKSESEDLQQVIASVLRNLSWRADVNSKKTLREVGSVKALMECALEVKKESTLKSVLSALWNLSAHCTENKADICAVDGALAFLVGTLTYRSQTNTLAIIESGGGILRNVSSLIATNEDHRQILRENNCLQTLLQHLKSHSLTIVSNACGTLWNLSARNPKDQEALWDMGAVSMLKNLIHSKHKMIAMGSAAALRNLMANRPAKYKDAN

SEQ ID NO: 229 - protein, 64 aa, Homo sapiens
GAMGTKYAVPDTSTYQYDESSGYYYDPTTGLYYDPNSQYYYNSLTQQYLYWDGEKETYVPAAES

SEQ ID NO: 230 - protein, 123 aa, Homo sapiens
GHMNSFVGLRVVAKWSSNGYFYSGKITRDVGAGKYKLLFDDGYECDVLGKDILLCDPIPLDTEVTALSEDEYFSAGVVKGHRKESGELYYSIEKEGQRKWYKRMAVILSLEQGNRLREQYGLG

SEQ ID NO: 231 - protein, 64 aa, Homo sapiens
GSMGTAGKWVLCNYDFQARNSSELSVKQRDVLEVLDDSRKWWKVRDPAGQEGYVPYNILTPYPG

SEQ ID NO: 232 - protein, 180 aa, Homo sapiens
IKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEF

SEQ ID NO: 233 - protein, 232 aa, Homo sapiens
  VARIANT 232: note = Any amino acid
SIPHLILELLKCEPDEPQVQAKIMAYLQQEKLSTFGLMCKMADQTLFSIVEWARSSIFFRELKVDDQMKLLQNCWSELLILDHIYRQVVHGKEGSIFLVTGQQVDYSIIASQAGATLNNLMSHAQELVAKLRSLQFDQREFVCLKFLVLFSLDVKNLENFQLVEGVQEQVNAALLDYTMCNYPQQTEKFGQLLLRLPEIRAISMQAEEYLYYKHLNGDVPYNNLLIEMLHAX

SEQ ID NO: 234 - protein, 94 aa, Homo sapiens
  VARIANT 93: note = Any amino acid
  VARIANT 94: note = Any amino acid
GSHMSDRKAVIKNADMSEDMQQDAVDCATQAMEKYNIEKDIAAYIKKEFDKKYNPTWHCIVGRNFGSYVTHETKHFIYFYLGQVAILLFKSGXX

SEQ ID NO: 235 - protein, 235 aa, Homo sapiens
  VARIANT 230: note = Any amino acid
  VARIANT 231: note = Any amino acid
  VARIANT 232: note = Any amino acid
  VARIANT 233: note = Any amino acid
  VARIANT 234: note = Any amino acid
  VARIANT 235: note = Any amino acid
HQLTAAQELIQQLVAAQLQCNKRSFSKVTPWPLSRDARQQRFAHFTELAIISVQEIVDFAKQVPGFLQLGREDQIALLKASTIEILLETARRYNHETECITFLKDFTYSKDDFHRAGLQVEFINPIFEFSRARRLGLDDAEYALLIAINIFSADRPNVQEPGRVEALQQPYVEALLSYTRIKRPQDQLRFPRLKLVSLRTLSSVHSEQVFALRLQDKKLPPLLSEIWDVXXXXXX

SEQ ID NO: 236 - protein, 88 aa, Homo sapiens
KKIDILLKAVGDTPIMKTKKWAVERTRTIQGLIDFIKKFLKLVASEQLFIYVNQSFAPSPDQEVGTLYECFGSDGKLVLHYCKSQAWG

SEQ ID NO: 237 - protein, 253 aa, Homo sapiens
  VARIANT 253: note = Any amino acid
PQLTPTLVSLLEVIEPEVLYAGYDSSVPDSTWRIMTTLNMLGGRQVIAAVKWAKAIPGFRNLHLDDQMTLLQYSWMALMAFALGWRSYRQSSANLLYFAPDLIINEQRMTLPCMYDQCKHMLYVSSELHRLQVSYEEYLCMKVLLLLSTIPKDGLKSQELFDEIRMTYIKELGAAIVAREGNSSQNWQRFYQLTKLLDSMHEVVENLLNYCFQTFLDKTMSIEFPEMLAEIITNQIPKYSNGNIKKLLFHQKX

SEQ ID NO: 238 - protein, 245 aa, Homo sapiens
  VARIANT 245: note = Any amino acid
SGGPNVPELILQLLQLEPDEDQVRARILGSLQEPTKSRPDQPAAFGLLCRMADQTFISIVDWARRCMVFKELEVADQMTLLQNCWSELLVFDHIYRQVQHGKEGSILLVTGQEVELTTVATQAGSLLHSLVLRAQELVLQLLALQLDRQEFVCLKFIILFSLDLKFLNNHILVKDAQEKANAALLDYTLCHYPHSGDKFQQLLLCLVEVRALSMQAKEYLYHKHLGNEMPRNNLLIEMLQAKQTX

SEQ ID NO: 239 - protein, 65 aa, Homo sapiens
  VARIANT 58: note = Any amino acid
  VARIANT 59: note = Any amino acid
  VARIANT 60: note = Any amino acid
  VARIANT 61: note = Any amino acid
  VARIANT 62: note = Any amino acid
  VARIANT 63: note = Any amino acid
  VARIANT 64: note = Any amino acid
  VARIANT 65: note = Any amino acid
ENMYVNKVWVQCENENCLKWRLLSSEDSAKVDHDEPWYCFMNTDSRYNNCSISEEDRXXXXXXXX

SEQ ID NO: 240 - protein, 198 aa, Homo sapiens
  VARIANT 186: note = Any amino acid
  VARIANT 187: note = Any amino acid
  VARIANT 188: note = Any amino acid
  VARIANT 189: note = Any amino acid
  VARIANT 190: note = Any amino acid
  VARIANT 191: note = Any amino acid
  VARIANT 192: note = Any amino acid
  VARIANT 193: note = Any amino acid
  VARIANT 194: note = Any amino acid
  VARIANT 195: note = Any amino acid
  VARIANT 196: note = Any amino acid
  VARIANT 197: note = Any amino acid
  VARIANT 198: note = Any amino acid
ENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEXXXXXXXXXXXXX

SEQ ID NO: 241 - protein, 187 aa, Homo sapiens
RPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRA

SEQ ID NO: 242 - protein, 105 aa, Homo sapiens
MVKQIESKTAFQEALDAAGDKLVVVDFSATWCGPAKMIKPFFHSLSEKYSNVIFLEVDVDDAQDVASEAEVKATPTFQFFKKGQKVGEFSGANKEKLEATINELV

SEQ ID NO: 243 - protein, 104 aa, Homo sapiens
GHPTEVLCLMNMVLPEELLDDEEYEEIVEDVRDECSKYGLVKSIEIPRPVDGVEVPGCGKIFVEFTSVFDCQKAMQGLTGRKFANRVVVTKYCDPDSYHRRDFW

SEQ ID NO: 244 - protein, 326 aa, Homo sapiens
HHHMLHLLEQIRAYCETCWEWQEAHNPMPAPVEHQICPAVCVLMKLSFDEEHRHAMNELGGLQAIAELLQVDCEMYGLTNDHYSITLRRYAGMALTNLTFGDVANKATLCSMKGCMRALVAQLKSESEDLQQVIASVLRNLSWRADVNSKKTLREVGSVKALMECALEVKKESTLKSVLSALWNLSAHCTENKADICAVDGALAFLVGTLTYRSQTNTLAIIESGGGILRNVSSLIATNEDHRQILRENNCLQTLLQHLKSHSLTIVSNACGTLWNLSARNPKDQEALWDMGAVSMLKNLIHSKHKMIAMGSAAALRNLMANRPAK

SEQ ID NO: 245 - protein, 171 aa, Homo sapiens
  VARIANT 168: note = Any amino acid
  VARIANT 169: note = Any amino acid
  VARIANT 170: note = Any amino acid
  VARIANT 171: note = Any amino acid
QSFLWNVFQRVDKDRSGVISDTELQQALSNGTWTPFNPVTVRSIISMFDRENKAGVNFSEFTGVWKYITDWQNVFRTYDRDNSGMIDKNELKQALSGFGYRLSDQFHDILIRKFDRQGRGQIAFDDFIQGCIVLQRLTDIFRRYDTDQDGWIQVSYEQYLSMVFSIVXXXX

SEQ ID NO: 246 - protein, 195 aa, Homo sapiens
ARLEEAVNRWVLKFYFHEALRAFRGSRYGDFRQIRDIMQALLVRPLGKEHTVSRLLRVMQCLSRIEEGENLDCSFDMEELTPLESAINVLEMIKTEFTLTEAVVESSRKLVKEAAVIICIKNKEFEKASKILKKHMPTTQKLRNDLLNIIREKNLAHPVIQNFSYETFQQKMLRFLESHLDDAEPYLLTMAKKAL

SEQ ID NO: 247 - protein, 357 aa, Homo sapiens
ATFISVQLKKTSEVDLAKPLVKFIQQTYPSGGEEQAQYCRAAEELSKLRRAAVGRPLDKHEGALETLLRYYDQICSIEPKFPFSENQICLTFTWKDAFDKGSLFGGSVKLALASLGYEKSCVLFNCAALASQIAAEQNLDNDEGLKIAAKHYQFASGAFLHIKETVLSALSREPTVDISPDTVGTLSLIMLAQAQEVFFLKATRDKMKDAIIAKLANQAADYFGDAFKQCQYKDTLPKEVFPVLAAKHCIMQANAEYHQSILAKQQKKFGEEIARLQHAAELIKTVASRYDEYVNVKDFSDKINRALAAAKKDNDFIYHDRVPDLKDLDPIGKATLVKSTPVNVPISQKFTDLFEKM

SEQ ID NO: 248 - protein, 197 aa, Homo sapiens
RQDLNFGQVVADVLCEFLEVAVHLILYVREVYPVGIFQKRKKYNVPVQMSCHPELNQYIQDTLHCVKPLLEKNDVEKVVVVILDKEHRPVEKFVFEITQPPLLSISSDSLLSHVEQLLAAFILKISVCDAVLDHNPPGCTFTVLVHTREAIQVIKDFPWILADEQDVHMHDPRLIPLKTMTSDILKMQLYVEERAHK

SEQ ID NO: 249 - protein, 83 aa, Homo sapiens
SEEEDKCKPMSYEEKRQLSLDINKLPGEKLGRVVHIIQSREPSLKNSNPDEIEIDFETLKPSTLRELERYVTSCLRKKRKPQA

SEQ ID NO: 250 - protein, 81 aa, Homo sapiens
GRLDLPPGFMFKVQAQHDYTATDTDELQLKAGDVVLVIPFQNPEEQDEGWLMGVKESDWNQHKKLEKCRGVFPENFTERVP

SEQ ID NO: 251 - protein, 147 aa, Homo sapiens
MSTAADLLRQGAACSVLYLTSVETESLTGPQAVARASSAALSCSPRPTPAVVHFKVSAQGITLTDNQRKLFFRRHYPVNSITFSSTDPQDRRWTNPDGTTSKIFGFVAKKPGSPWENVCHLFAELDPDQPAGAIVTFITKVLLGQRK

SEQ ID NO: 252 - protein, 437 aa, Homo sapiens
  VARIANT 433: note = Any amino acid
  VARIANT 434: note = Any amino acid
  VARIANT 435: note = Any amino acid
  VARIANT 436: note = Any amino acid
  VARIANT 437: note = Any amino acid
SVPVYCLCRLPYDVTRFMIECDMCQDWFHGSCVGVEEEKAADIDLYHCPNCEVLHGPSIMKKKPVKTGSPTFVRELRSRTFDSSDEVILKPTGNQLTVEFLEENSFSVPILVLKKDGLGMTLPSPSFTVRDVEHYVGSDKEIDVIDVTRQADCKMKLGDFVKYYYSGKREKVLNVISLEFSDTRLSNLVETPKIVRKLSWVENLWPEECVFERPNVQKYCLMSVRDSYTDFHIDFGGTSVWYHVLKGEKIFYLIRPTNANLTLFECWSSSSNQNEMFFGDQVDKCYKCSVKQGQTLFIPTGWIHAVLTPVDCLAFGGNFLHSLNIEMQLKAYEIEKRLSTADLFRFPNFETICWYVGKHILDIFRGLRENRRHPASYLVHGGKALNLAFRAWTRKEALPDHEDEIPETVRTVQLIKDLAREIRLVEDIFQQNXXXXX

SEQ ID NO: 253 - protein, 149 aa, Homo sapiens
  VARIANT 146: note = Any amino acid
  VARIANT 147: note = Any amino acid
  VARIANT 148: note = Any amino acid
  VARIANT 149: note = Any amino acid
LTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAKXXXX

SEQ ID NO: 254 - protein, 229 aa, Homo sapiens
  VARIANT 229: note = Any amino acid
SLTEIEHLVQSVCKSYRETCQLRLEDLLRQRSNIFSREEVTGYQRKSMWEMWERCAHHLTEAIQYVVEFAKRLSGFMELCQNDQIVLLKAGAMEVVLVRMCRAYNADNRTVFFEGKYGGMELFRALGCSELISSIFDFSHSLSALHFSEDEIALYTALVLINAHRPGLQEKRKVEQLQYNLELAFHHHLCKTHRQSILAKLPPAGKLASLCSQHVERLQIFQHLHPIVX

SEQ ID NO: 255 - protein, 151 aa, Homo sapiens
AELCESLLTWIQTFNVDAPCQTVEDLTNGVVMAQVLQKIDPAYFDENWLNRIKTEVGDNWRLKISNLKKILKGILDYNHEILGQQINDFTLPDVNLIGEHSDAAELGRMLQLILGCAVNCEQKQEYIQAIMMMEESVQHVVMTAIQELMSK

SEQ ID NO: 256 - protein, 187 aa, Homo sapiens
GDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQHHNLLVCSVSGFYPGSIEVRWFRNGQEEKAGVVSTGLIQNGDWTFQTLVMLETVPRSGEVYTCQVEHPSVTSPLTVEWRA

SEQ ID NO: 257 - protein, 145 aa, Homo sapiens
  VARIANT 143: note = Any amino acid
  VARIANT 144: note = Any amino acid
  VARIANT 145: note = Any amino acid
GAMADKRGDGLHGIVSCTACGQQVNHFQKDSIYRHPSLQVLICKNCFKYYMSDDISRDSDGMDEQCRWCAEGGNLICCDFCHNAFCKKCILRNLGRKELSTIMDENNQWYCYICHPEPLLDLVTACNSVFENLEQLLQQNKKXXX

SEQ ID NO: 258 - protein, 148 aa, Homo sapiens
ADQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAK

SEQ ID NO: 259 - protein, 89 aa, Homo sapiens
QINQVRPKLPLLKILHAAGAQGEMFTVKEVMHYLGQYIMVKQLYDQQEQHMVYCGGDLLGELLGRQSFSVKDPSPLYDMLRKNLVTLAT

SEQ ID NO: 260 - protein, 147 aa, Homo sapiens
  VARIANT 144: note = Any amino acid
  VARIANT 145: note = Any amino acid
  VARIANT 146: note = Any amino acid
  VARIANT 147: note = Any amino acid
TEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAXXXX

SEQ ID NO: 261 - protein, 79 aa, Homo sapiens
GPLGSLTASMLASAPPQEQKQMLGERLFPLIQAMHPTLAGKITGMLLEIDNSELLHMLESPESLRSKVDEAVAVLQAHQ

SEQ ID NO: 262 - protein, 384 aa, Homo sapiens
  VARIANT 384: note = Any amino acid
FEDTVEERVINEEYKIWKKNTPFLYDLVMTHALQWPSLTVQWLPEVTKPEGKDYALHWLVLGTHTSDEQNHLVVARVHIPNDDTGKIECEIKINHEGEVNRARYMPQNPHIIATKTPSSDVLVFDYTKHPAKPDPSGECNPDLRLRGHQKEGYGLSWNSNLSGHLLSASDDHTVCLWDINAGPKEGKIVDAKAIFTGHSAVVEDVAWHLLHESLFGSVADDQKLMIWDTRSNTTSKPSHLVDAHTAEVNCLSFNPYSEFILATGSADKTVALWDLRNLKLKLHTFESHKDEIFQVHWSPHNETILASSGTDRRLNVWDLSKIGEEQSAEDAEDGPPELLFIHGGHTAKISDFSWNPNEPWVICSVSEDNIMQIWQMAENIYNDX

SEQ ID NO: 263 - protein, 178 aa, Homo sapiens
EEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKQWEF

SEQ ID NO: 264 - protein, 110 aa, Homo sapiens
QSALTQPASVSGSPGQSITISCTGTSSDIGAYNYVSWYQQYPGKAPKLLIYDVSNRPSGISNRFSGSKSGDTASLTISGLQAEDEADYYCSSFANSGPLFGGGTKVTVLG

SEQ ID NO: 265 - protein, 272 aa, Homo sapiens
  VARIANT 272: note = Any amino acid
ESADLRALAKHLYDSYIKSFPLTKAKARAILTGKTTDKSPFVIYDMNSLMMGEDKIKFKHITPLQEQSKEVAIRIFQGCQFRSVEAVQEITEYAKSIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIYKDLYX

SEQ ID NO: 266 - protein, 278 aa, Homo sapiens
  VARIANT 277: note = Any amino acid
  VARIANT 278: note = Any amino acid
GSHSMRYFYTAMSRPGRGEPRFIAVGYVDDTQFVRFDSDAASPRTEPRAPWIEQEGPEYWDRNTQIFKTNTQTYRESLRNLRGYYNQSEAGSHIIQRMYGCDLGPDGRLLRGHDQSAYDGKDYIALNEDLSSWTAADTAAQITQRKWEAARVAEQRRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEPXX

SEQ ID NO: 267 - protein, 274 aa, Homo sapiens
GSHSMRYFYTAMSRPGRGEPRFIAVGYVDDTQFVRFDSDAASPRMAPRAPWIEQEGPEYWDGETRNMKASAQTYRENLRIALRYYNQSEAGSHIIQVMYGCDVGPDGRLLRGHNQYAYDGKDYIALNEDLSSWTAADTAAQITQRKWEAARVAEQLRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRW

SEQ ID NO: 268 - protein, 232 aa, Homo sapiens
RITLKESGPPLVKPTQTLTLTCSFSGFSLSDFGVGVGWIRQPPGKALEWLAIIYSDDDKRYSPSLNTRLTITKDTSKNQVVLVMTRVSPVDTATYFCAHRRGPTTLFGVPIARGPVNAMDVWGQGITVTISSTSTKGPSVFPLAPSSAGGAAALGCLVKDYFPEPVTVSWNSGALTSGVHTFPAVLQSSGLYSLSSVVTVPSSSLGTQTYTCNVNHKPSNTKVDKRVEPKSC

SEQ ID NO: 269 - protein, 276 aa, Homo sapiens
GSHSMRYFYTAMSRPGRGEPRFIAVGYVDDTQFVRFDSDAASPRTEPRPPWIEQEGPEYWDRNTQIFKTNTQTYRESLRNLRGYYNQSEAGSHIIQRMYGCDLGPDGRLLRGHDQSAYDGKDYIALNEDLSSWTAADTAAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEP

SEQ ID NO: 270 - protein, 276 aa, Homo sapiens
GSHSMRYFDTAMSRPGRGEPRFISVGYVDDTQFVRFDSDAASPREEPRAPWIEQEGPEYWDRNTQIFKTNTQTDRESLRNLRGYYNQSEAGSHTLQSMYGCDVGPDGRLLRGHNQYAYDGKDYIALNEDLRSWTAADTAAQITQRKWEAARVAEQDRAYLEGTCVEWLRRYLENGKDTLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 271 - protein, 165 aa, Homo sapiens
MVNPTVFFDIAVDGEPLGRVSFELFADKVPKTAENFRALSTGEKGFGYKGSCFHRIIPGFMCQGGDFTRHNGTGGKSIYGEKFEDENFILKHTGPGILSMANAGPNTNGSQFFICTAKTEWLDGKHVVFGKVKEGMNIVEAMERFGSRNGKTSKKITIADCGQLE

SEQ ID NO: 272 - protein, 336 aa, Homo sapiens
NTIQQLMMILNSASDQPSENLISYFNNCTVNPKESILKRVKDIGYIFKEKFAKAVGQGCVEIGSQRYKLGVRLYYRVMESMLKSEEERLSIQNFSKLLNDNIFHMSLLACALEVVMATYSRSTGTDLSFPWILNVLNLKAFDFYKVIESFIKAEGNLTREMIKHLERCEHRIMESLAWLSDSPLFDLIKQSKDKSTSLSLFYKKVYRLAYLRLNTLCERLLSEHPELEHIIWTLFQHTLQNEYELMRDRHLDQIMMCSMYGICKVKNIDLKFKIIVTAYKDLPHAVQETFKRVLIKEEEYDSIIVFYNSVFMQRLKTNILQYASTRPPTLSPIPHI

SEQ ID NO: 273 - protein, 141 aa, Homo sapiens
TSLSLFYKKVYRLAYLRLNTLCERLLSEHPELEHIIWTLFQHTLQNEYELMRDRHLDQIMMCSMYGICKVKNIDLKFKIIVTAYKDLPHAVQETFKRVLIKEEEYDSIIVFYNSVFMQRLKTNILQYASTRPPTLSPIPHI

SEQ ID NO: 274 - protein, 322 aa, Homo sapiens
  VARIANT 306: note = Any amino acid
  VARIANT 307: note = Any amino acid
  VARIANT 308: note = Any amino acid
  VARIANT 309: note = Any amino acid
  VARIANT 310: note = Any amino acid
  VARIANT 311: note = Any amino acid
  VARIANT 312: note = Any amino acid
  VARIANT 313: note = Any amino acid
  VARIANT 314: note = Any amino acid
  VARIANT 315: note = Any amino acid
  VARIANT 316: note = Any amino acid
  VARIANT 317: note = Any amino acid
  VARIANT 318: note = Any amino acid
  VARIANT 319: note = Any amino acid
  VARIANT 320: note = Any amino acid
  VARIANT 321: note = Any amino acid
  VARIANT 322: note = Any amino acid
PVKPNYALKFTLAGHTKAVSSVKFSPNGEWLASSSADKLIKIWGAYDGKFEKTISGHKLGISDVAWSSDSNLLVSASDDKTLKIWDVSSGKCLKTLKGHSNYVFCCNFNPQSNLIVSGSFDESVRIWDVKTGKCLKTLPAHSDPVSAVHFNRDGSLIVSSSYDGLCRIWDTASGQCLKTLIDDDNPPVSFVKFSPNGKYILAATLDNTLKLWDYSKGKCLKTYTGHKNEKYCIFANFSVTGGKWIVSGSEDNLVYIWNLQTKEIVQKLQGHTDVVISTACHPTENIIASAALENDKTIKLWKSDCXXXXXXXXXXXXXXXXX

SEQ ID NO: 275 - protein, 183 aa, Homo sapiens
  VARIANT 174: note = Any amino acid
  VARIANT 175: note = Any amino acid
  VARIANT 176: note = Any amino acid
  VARIANT 177: note = Any amino acid
  VARIANT 178: note = Any amino acid
  VARIANT 179: note = Any amino acid
  VARIANT 180: note = Any amino acid
  VARIANT 181: note = Any amino acid
  VARIANT 182: note = Any amino acid
  VARIANT 183: note = Any amino acid
EFETIERFMDCRIGRKGATGATTTIYAVEADGDPNAGFEKNKEPGEIQYLIKWKGWSHIHNTWETEETLKQQNVRGMKKLDNYKKKDQETKRWLKNASPEDVEYYNCQQELTDDLHKQYQIVERIIAHSNQKSAAGYPDYYCKWQGLPYSECSWEDGALISKKFQACIDEYFSXXXXXXXXXX

SEQ ID NO: 276 - protein, 179 aa, Homo sapiens
EHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDA

SEQ ID NO: 277 - protein, 106 aa, Homo sapiens
SIQAEEWYFGKLGRKDAERQLLSFGNPRGTFLIRESETTKGAYSLSIRDWDDMKGDHVKHYKIRKLDNGGYYITTRAQFETLQQLVQHYSERAAGLSSRLVVPSHK

SEQ ID NO: 278 - protein, 298 aa, Homo sapiens
  VARIANT 296: note = Any amino acid
  VARIANT 297: note = Any amino acid
  VARIANT 298: note = Any amino acid
DEKVFTKELDQWIEQLNECKQLSESQVKSLCEKAKEILTKESNVQEVRCPVTVCGDVHGQFHDLMELFRIGGKSPDTNYLFMGDYVDRGYYSVETVTLLVALKVRYRERITILRGNHESRQITQVYGFYDECLRKYGNANVWKYFTDLFDYLPLTALVDGQIFCLHGGLSPSIDTLDHIRALDRLQEVPHEGPMCDLLWSDPDDRGGWGISPRGAGYTFGQDISETFNHANGLTLVSRAHQLVMEGYNWCHDRNVVTIFSAPNYCYRCGNQAAIMELDDTLKYSFLQFDPAPRRGXXX

SEQ ID NO: 279 - protein, 123 aa, Homo sapiens
  VARIANT 122: note = Any amino acid
  VARIANT 123: note = Any amino acid
LYSSSDDVIELTPSNFNREVIQSDSLWLVEFYAPWCGHAQRLTPEWKKAATALKDVVKVGAVDADKHHSLGGQYGVQGFPTIKIFGSNKNRPEDYQGGRTGEAIVDAALSALRQLVKDRLGXX

SEQ ID NO: 280 - protein, 210 aa, Homo sapiens
  VARIANT 210: note = Any amino acid
NEQEKELQRRLKRLYPAVDEQETPLPRSWSPKDKFSYIGLSQNNLRVHYKGHGKTPKDAASVRATHPIPAACGIYYFEVKIVSKGRDGYMGIGLSAQGVNMNRLPGWDKHSYGYHGDDGHSFCSSGTGQPYGPTFTTGDVIGCCVNLINNTCFYTKNGHSLGIAFTDLPPNLYPTVGLQTPGEVVDANFGQHPFVFDIEDYMREWRTKIX

SEQ ID NO: 281 - protein, 122 aa, Homo sapiens
MPSEKTFKQRRTFEQRVEDVRLIREQHPTKIPVIIERYKGEKQLPVLDKTKFLVPDHVNMSELIKIIRRRLQLNANQAFFLLVNGHSMVSVSTPISEVYESEKDEDGFLYMVYASQETFGMK

SEQ ID NO: 282 - protein, 236 aa, Homo sapiens
  VARIANT 113: note = Any amino acid
  VARIANT 235: note = Any amino acid
  VARIANT 236: note = Any amino acid
SLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKXVEGMVEIFDMLLATSSRFRMMNLQGEEFVCLKSIILLNSGVYLEEKDHIHRVLDKITDTLIHLMAKAGLTLQQQHQRLAQLLLILSHIRHMSNKGMEHLYSMKCKNVVPLYDLLLEMLDAHXX

SEQ ID NO: 283 - protein, 239 aa, Homo sapiens
  VARIANT 239: note = Any amino acid
SLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLEEKDHIHRVLDKITDTLIHLMAKAGLTLQQQHQRLAQLLLILSHIRHMSNKGMEHLYSMKAKNVVPLYDLLLEMLDAHRLX

SEQ ID NO: 284 - protein, 362 aa, Homo sapiens
  VARIANT 362: note = Any amino acid
DFQGMLEYKKEDEQKLVKNLILELKPRGVAVNLIPGLPAYILFMCVRHADYLNDDQKVRSLLTSTINSIKKVLKKRGDDFETVSFWLSNTCRFLHCLKQYSGEEGFMKHNTSRQNEHCLTNFDLAEYRQVLSDLAIQIYQQLVRVLENILQPMIVSGMLEHEGTYTLDSILRQLNSFHSVMCQHGMDPELIKQVVKQMFYIIGAITLNNLLLRKDMCSWSKGMQIRYNVSQLEEWLRDKNLMNSGAKETLEPLIQAAQLLQVKKKTDDDAEAICSMCNALTTAQIVKVLNLYTPVNEFEERVSVSFIRTIQMRLRDRKDSPQLLMDAKHIFPVTFPFNPSSLALETIQIPASLGLGFISRVX

SEQ ID NO: 285 - protein, 263 aa, Homo sapiens
VPTEEVSLEVLLSNGQKVLVNVLTSDQTEDVLEAVAAKLDLPDDLIGYFSLFLVREKEDGAFSFVRKLQEFELPYVSVTSLRSQEYKIVLRKSYWDSAYDDDVMENRVGLNLLYAQTVSDIERGWILVTKEQHRQLKSLQEKVSKKEFLRLAQTLRHYGYLRFDACVADFPEKDCPVVVSAGNSELSLQLREGSFRVTRMRCWRVTSSVPLPSGGRGEVRLELAFEYLMSKDRLQWVTITSPQAIMMSICLQSMVDELMVKKS

SEQ ID NO: 286 - protein, 95 aa, Homo sapiens
QIPASEQETLVRPKPLLLKLLKSVGAQKDTYTMKEVLFYLGQYIATKRLYDEKQQHIVYCSNDLLGDLFGVPSFSVKEHRKIYTMIYRNLVVVNQ

SEQ ID NO: 287 - protein, 252 aa, Homo sapiens
IVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 288 - protein, 275 aa, Homo sapiens [sequence omitted]

SEQ ID NO: 289 - protein, 275 aa, Homo sapiens [sequence omitted]

SEQ ID NO: 290 - protein, 96 aa, Homo sapiens
GGSMRPPIIIHRAGKKYGFTLRAIRVYMGDSDVYTVHHMVWHVEDGGPASEAGLRQGDLITHVNGEPVHGLVHTEVVELILKSGNKVAISTTPLEN

SEQ ID NO: 291 - protein, 305 aa, Homo sapiens
  VARIANT 305: note = Any amino acid
PGTVDKKMVEKCWKLMDKVVRLCQNPKLALKNSPPYILDLLPDTYQHLRTILSRYEGKMETLGENEYFRVFMENLMKKTKQTISLFKEGKERMYEENSQPRRNLTKLSLIFSHMLAELKGIFPSGLFQGDTFRITKADAAEFWRKAFGEKTIVPWKSFRQALHEVHPISSGLEAMALKSTIDLTCNDYISVFEFDIFTRLFQPWSSLLRNWNSLAVTHPGYMAFLTYDEVKARLQKFIHKPGSYIFRLSCTRLGQWAIGYVTADGNILQTIPHNKPLFQALIDGFREGFYLFPDGRNQNPDLTGX

SEQ ID NO: 292 - protein, 233 aa, Homo sapiens
GGYVAPKAVWLPAVKAKGLEISGTFTHRQGHIYMEMNFTNKALQHMTDFAIQFNKNSFGVIPSTPLAIHTPLMPNQSIDVSLPLNTLGPVMKMEPLNNLQVAVKNNIDVFYFSCLIPLNVLFVEDGKMERQVFLATWKDIPNENELQFQIKECHLNADTVSSKLQNNNVYTIAKRNVEGQDMLYQSLKLTNGIWILAELRIQPGNPNYTLSLKCRAPEVSQYIYQVYDSILKN

SEQ ID NO: 293 - protein, 143 aa, Homo sapiens
  VARIANT 140: note = Any amino acid
  VARIANT 141: note = Any amino acid
  VARIANT 142: note = Any amino acid
  VARIANT 143: note = Any amino acid
EEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNKLTDEEVDEMIREADIDGDGQVNYEEFVQMMTAXXXX

SEQ ID NO: 294 - protein, 101 aa, Homo sapiens
  VARIANT 101: note = Any amino acid
EMKPHPWFFGKIPRAKAEEMLSKQRHDGAFLIRESESAPGDFSLSVKFGNDVQHFKVLRDGAGKYFLWVVKFNSLNELVDYHRSTSVSRNQQIFLRDIEQX

SEQ ID NO: 295 - protein, 373 aa, Homo sapiens
  VARIANT 363: note = Any amino acid
  VARIANT 364: note = Any amino acid
  VARIANT 365: note = Any amino acid
  VARIANT 366: note = Any amino acid
  VARIANT 367: note = Any amino acid
  VARIANT 368: note = Any amino acid
  VARIANT 369: note = Any amino acid
  VARIANT 370: note = Any amino acid
  VARIANT 371: note = Any amino acid
  VARIANT 372: note = Any amino acid
  VARIANT 373: note = Any amino acid
KYSFKCVNSLKEDHNQPLFGVQFNWHSKEGDPLVFATVGSNRVTLYECHSQGEIRLLQSYVDADADENFYTCAWTYDSNTSHPLLAVAGSRGIIRIINPITMQCIKHYVGHGNAINELKFHPRDPNLLLSVSKDHALRLWNIQTDTLVAIFGGVEGHRDEVLSADYDLLGEKIMSCGMDHSLKLWRINSKRMMNAIKESYDYNPNKTNRPFISQKIHFPDFSTRDIHRNYVDCVRWLGDLILSKSCENAIVCWKPGKMEDDIDKIKPSESNVTILGRFDYSQCDIWYMRFSMDFWQKMLALGNQVGKLYVWDLEVEDPHKAKCTTLTHHKCGAAIRQTSFSRDSSILIAVCDDASIWRWDRLXXXXXXXXXXX

SEQ ID NO: 296 - protein, 95 aa, Homo sapiens
HKIPVEADFLYAYSTAPGYYSWRNSKDGSWFIQSLCAMLKQYADKLEFMHILTRVNRKVATEFESFSFDATFHAKKQIPCIVSMLTKELYFYHLE

SEQ ID NO: 297 - protein, 226 aa, Homo sapiens
LALSLTADQMVSALLDAEPPILYSEYDPTRPSEASMMGLLTNLADRELVHMINWAKRVPGFVDLTLHDQVHLLECAWLEILMIGLVWRSMEHPGKLLFAPNLLLDRNQGKCVGMVEIFDMLLATSSRFRMMNLQGEEFVCLKSIILLNSGVYTDHIHRVLDKITDTLIHLMAKAGLTLQQQHQRLAQLLLILSHIRHMSNKGMEHLYSMKCKNLYGLLLEMLDAHR

SEQ ID NO: 298 - protein, 473 aa, Homo sapiens
  VARIANT 470: note = Any amino acid
  VARIANT 471: note = Any amino acid
  VARIANT 472: note = Any amino acid
  VARIANT 473: note = Any amino acid
YQEPTDPKFPQQWYLSGVTQRDLNVKAAWAQGYTGHGIVVSILDDGIEKNHPDLAGNYDPGASFDVNDQDPDPQPRYTQMNDNRHGTRCAGEVAAVANNGVCGVGVAYNARIGGVRMLDGEVTDAVEARSLGLNPNHIHIYSASWGPEDDGKTVDGPARLAEEAFFRGVSQGRGGLGSIFVWASGNGGREHDSCNCDGYTNSIYTLSISSATQFGNVPWYSEACSSTLATTYSSGNQNEKQIVTTDLRQKCTESHTGTSASAPLAAGIIALTLEANKNLTWRDMQHLVVQTSKPAHLNANDWATNGVGRKVSHSYGYGLLDAGAMVALAQNWTTVAPQRKCIIDILTEPKDIGKRLEVRKTVTACLGEPNHITRLEHAQARLTLSYNRRGDLAIHLVSPMGTRSTLLAARPHDYSADGFNDWAFMTTHSWDEDPSGEWVLEIENTSEANNYGTLTKFTLVLYGTASGSLXXXX

SEQ ID NO: 299 - protein, 204 aa, Homo sapiens
  VARIANT 202: note = Any amino acid
  VARIANT 203: note = Any amino acid
  VARIANT 204: note = Any amino acid
AAALEGPYPVEEIRLPRAGGPLGLSIVGGSDHSSHPFGVQEPGVFISKVLPRGLAARSGLRVGDRILAVNGQDVRDATHQEAVSALLRPCLELSLLVRRDPAPPGLRELCIQKAPGERLGISIRGGARGHAGNPRDPTDEGIFISKVSPTGAAGRDGRLRVGLRLLEVNQQSLLGLTHGEAVQLLRSVGDTLTVLVCDGFEXXX

SEQ ID NO: 300 - protein, 244 aa, Homo sapiens
  VARIANT 244: note = Any amino acid
ASLTEIEHLVQSVCKSYRETCQLRLEDLLRQRSNIFSREEVTGYQRKSMWEMWERCAHHLTEAIQYVVEFAKRLSGFMELCQNDQIVLLKAGAMEVVLVRMCRAYNADNRTVFFEGKYGGMELFRALGCSELISSIFDFSHSLSALHFSEDEIALYTALVLINAHRPGLQEKRKVEQLQYNLELAFHHHLCKTHRQSILAKLPPKGKLRSLCSQHVERLQIFQHLHPIVVQAAFPPLYKELFSX

SEQ ID NO: 301 - protein, 275 aa, Homo sapiens
  VARIANT 275: note = Any amino acid
GKLSLQDVAELIRARACQRVVVMVGAGISTPSGIPDFRSPGSGLYSNLQQYDLPYPEAIFELPFFFHNPKPFFTLAKELYPGNYKPNVTHYFLRLLHDKGLLLRLYTQNIDGLERVSGIPASKLVEAHGTFASATCTVCQRPFPGEDIRADVMADRVPRCPVCTGVVKPDIVFFGEPLPQRFLLHVVDFPMADLLLILGTSLEVEPFASLTEAVRSSVPRLLINRDLVGPLAWHPRSRDVAQLGDVVHGVESLVELLGWTEEMRDLVQRETGKLX

SEQ ID NO: 302 - protein, 101 aa, Homo sapiens
  VARIANT 101: note = Any amino acid
QLWFHGRISREESQRLIGQQGLVDGLFLVRESQRNPQGFVLSLCHLQKVKHYLILPSEEEGRLYFSMDDGQTRFTDLLQLVEFHQLNRGILPCLLRHCCTX

SEQ ID NO: 303 - protein, 247 aa, Homo sapiens
IIGGEFTTIENQPWFAAIYRRHRGGSVTYVCGGSLISPCWVISATHCFIDYPKKEDYIVYLGRSRLNSNTQGEMKFEVENLILHKDYSADTLAYHNDIALLKIRSKEGRCAQPSRTIQTIALPSMYNDPQFGTSCEITGFGKEQSTDYLYPEQLKMTVVKLISHRECQQPHYYGSEVTTKMLCAADPQWKTDSCQGDSGGPLVCSLQGRMTLTGIVSWGRGCALKDKPGVYTRVSHFLPWIRSHTKE

SEQ ID NO: 304 - protein, 161 aa, Homo sapiens
GMWDETELGLYKVNEYVDARDTNMGAWFEAQVVRVTRKAPSRDEPCSSTSRPALEEDVIYHVKYDDYPENGVVQMNSRDVRARARTIIKWQDLEVGQVVMLNYNPDNPKERGFWYDAEISRKRETRTARELYANVVLGDDSLNDCRIIFVDEVFKIERPGE

SEQ ID NO: 305 - protein, 239 aa, Homo sapiens
IVGGTNSSWGEWPWQVSLQVKLTAQRHLCGGSLIGHQWVLTAAHCFDGLPLQDVWRIYSGILNLSDITKDTPFSQIKEIIIHQNYKVSEGNHDIALIKLQAPLNYTEFQKPISLPSKGDTSTIYTNCWVTGWGFSKEKGEIQNILQKVNIPLVTNEECQKRYQDYKITQRMVCAGYKEGGKDACKGDSGGPLVCKHNGMWRLVGITSWGEGCARREQPGVYTKVAEYMDWILEKTQSSD

SEQ ID NO: 306 - protein, 149 aa, Homo sapiens
  VARIANT 146: note = Any amino acid
  VARIANT 147: note = Any amino acid
  VARIANT 148: note = Any amino acid
  VARIANT 149: note = Any amino acid
ADQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMXXXX

SEQ ID NO: 307 - protein, 108 aa, Homo sapiens
GDKNFPRTVMVNLNIHNSDYYDRSTSPWNLHRNEDPERYPSVIWEAKCRHLGCINADGNVDYHMNSVPIQQEILVLRREPPHSPNSFRLEKILVSVGCTCVTPIVHHV

SEQ ID NO: 308 - protein, 165 aa, Homo sapiens; identical to SEQ ID NO: 271

SEQ ID NO: 309 - protein, 228 aa, Homo sapiens
EVKLVESGGGLVQPGGSLKLSCATSGFTFSDYYMYWVRQNSEKRLEWVAFISNGGGSAFYADIVKGRFTISRDNAKNTLYLQMSRLKSEDTAMYYCTRHTLYDTLYGNYPVWFADWGQGTLVTVSAAKTTPPSVYPLAPGSAAQTNSMVTLGCLVKGYFPEPVTVTWNSGSLSSGVHTFPAVLQSDLYTLSSSVTVPSSSRPSETVTCNVAHPASSTKVDKKIVPRDC

SEQ ID NO: 310 - protein, 304 aa, Homo sapiens; identical to SEQ ID NO: 170

SEQ ID NO: 311 - protein, 4 aa, unidentified
  SITE 1: note = Acetylated residue
  SITE 4: note = Fluoromethylketone modified residue
DVAD

SEQ ID NO: 312 - skipped

SEQ ID NO: 313 - protein, 5 aa, unidentified
YQSKL

SEQ ID NO: 314 - protein, 5 aa, unidentified
  VARIANT 4: note = Any amino acid
TDYXR

SEQ ID NO: 315 - protein, 5 aa, unidentified
  SITE 1: note = Acetylated phosphotyrosine
YEEIE

SEQ ID NO: 316 - protein, 5 aa, unidentified
KLNYL

SEQ ID NO: 317 - protein, 5 aa, unidentified
ASVSA

SEQ ID NO: 318 - protein, 5 aa, unidentified
  VARIANT 1: note = Any amino acid
XVPML

SEQ ID NO: 319 - skipped

SEQ ID NO: 320 - protein, 5 aa, unidentified
PAMAM

SEQ ID NO: 321-322 - skipped (2 entries)

SEQ ID NO: 323 - protein, 5 aa, unidentified
ASVSA

SEQ ID NO: 324 - protein, 5 aa, unidentified
  VARIANT 4: note = Any amino acid
ARTXQ

SEQ ID NO: 325 - protein, 5 aa, unidentified
  VARIANT 4: note = Any amino acid
ARTXQ

SEQ ID NO: 326 - protein, 5 aa, unidentified
QDTRL

SEQ ID NO: 327 - protein, 5 aa, unidentified
QDTRL

SEQ ID NO: 328 - protein, 5 aa, unidentified
  VARIANT 4: note = Any amino acid
ARTXQ

SEQ ID NO: 329 - protein, 5 aa, unidentified
  VARIANT 4: note = Any amino acid
GGVXK

SEQ ID NO: 330 - skipped

SEQ ID NO: 331 - protein, 4 aa, unidentified
  SITE 4: note = Amidated residue
LHSA

SEQ ID NO: 332 - protein, 5 aa, unidentified
  VARIANT 5: note = Any amino acid
AIETX

SEQ ID NO: 333 - protein, 5 aa, unidentified
ALDKW

SEQ ID NO: 334 - skipped

SEQ ID NO: 335 - protein, 5 aa, unidentified
  VARIANT 3: note = Any amino acid
HRXVL

SEQ ID NO: 336-339 - skipped (4 entries)

SEQ ID NO: 340 - protein, 5 aa, unidentified
VMGPW

SEQ ID NO: 341 - protein, 5 aa, unidentified
PTSII

SEQ ID NO: 342 - protein, 4 aa, unidentified
  SITE 1: note = Acetylated residue
  SITE 4: note = Chloromethylketone modified residue
DEVD

SEQ ID NO: 343 - protein, 6 aa, unidentified
  VARIANT 6: note = Any amino acid
TVASSX

SEQ ID NO: 344 - protein, 6 aa, unidentified
HAGPIA

SEQ ID NO: 345 - protein, 6 aa, unidentified
  VARIANT 1: note = Any amino acid
  VARIANT 2: note = Any amino acid
XXVNVP

SEQ ID NO: 346 - protein, 6 aa, unidentified
STNPFR

SEQ ID NO: 347 - protein, 6 aa, unidentified
GDYMNM

SEQ ID NO: 348 - protein, 6 aa, unidentified
  VARIANT 3: note = Any amino acid
SHXYPA

SEQ ID NO: 349 - protein, 6 aa, unidentified
RRETQV

SEQ ID NO: 350 - protein, 6 aa, unidentified
ELKERY

SEQ ID NO: 351 - protein, 6 aa, unidentified
  VARIANT 4: note = Any amino acid
ARTXQT

SEQ ID NO: 352 - protein, 6 aa, unidentified
  SITE 3: note = Phosphotyrosine
VIYFVP

SEQ ID NO: 353 - protein, 6 aa, unidentified
  VARIANT 1: note = Any amino acid
  VARIANT 6: note = Any amino acid
XVRPRX

SEQ ID NO: 354 - protein, 6 aa, unidentified
RRRRSW

SEQ ID NO: 355 - protein, 6 aa, unidentified
  VARIANT 3: note = Any amino acid
WPXSII

SEQ ID NO: 356 - protein, 6 aa, unidentified
YPTSII

SEQ ID NO: 357 - protein, 6 aa, unidentified
VQDTRL

SEQ ID NO: 358 - protein, 7 aa, unidentified
  SITE 2: note = Phosphoserine
  SITE 5: note = Phosphoserine
YSPTSPS

SEQ ID NO: 359 - protein, 7 aa, unidentified
  VARIANT 7: note = Any amino acid
GGKLSKX

SEQ ID NO: 360 - protein, 7 aa, unidentified
  VARIANT 4: note = Any amino acid
ARTXQTA

SEQ ID NO: 361 - protein, 7 aa, unidentified
SLIPPDK

SEQ ID NO: 362 - protein, 7 aa, unidentified
KRRRHPS

SEQ ID NO: 363 - protein, 7 aa, unidentified
RWPTSIL

SEQ ID NO: 364 - protein, 7 aa, unidentified
NRLLLTG

SEQ ID NO: 365 - protein, 8 aa, unidentified
  SITE 4: note = 2-amino-3-(4-phosphonomethyl-phenyl)-propionic acid
EGDXEEVL

SEQ ID NO: 366 - protein, 8 aa, unidentified
PQPVDSWV

SEQ ID NO: 367 - protein, 8 aa, unidentified
PQPVDSWV

SEQ ID NO: 368 - protein, 8 aa, unidentified
  VARIANT 6: note = Any amino acid
KQTARXST

SEQ ID NO: 369 - protein, 8 aa, unidentified
SRWPTSIL

SEQ ID NO: 370 - protein, 8 aa, unidentified
DTLFSQHY

SEQ ID NO: 371 - protein, 9 aa, unidentified
LPPLDITPY

SEQ ID NO: 372 - protein, 9 aa, unidentified
  SITE 9: note = 3-Iodotyrosine
PPPPPPPPY

SEQ ID NO: 373 - protein, 8 aa, unidentified
  SITE 4: note = Phosphotyrosine
  SITE 8: note = Amidated residue
LEFYMDYE

SEQ ID NO: 374 - protein, 9 aa, unidentified
ARAAAAAAA

SEQ ID NO: 375 - skipped

SEQ ID NO: 376 - protein, 9 aa, unidentified
EEYLKAWTF

SEQ ID NO: 377 - protein, 8 aa, unidentified
  SITE 1: note = (7-methoxy-2-oxo-2H-chromen-4-yl) acetic acid
  SITE 5: note = Iodo-phenylalanine
  SITE 6: note = 3-[(2,4-dinitrophenyl)amino]-L-alanine
  SITE 8: note = Amidated residue
XPLGXXAR

SEQ ID NO: 378 - protein, 10 aa, unidentified
GYENPTYKFF

SEQ ID NO: 379 - protein, 10 aa, unidentified
  VARIANT 4: note = Any amino acid
  VARIANT 10: note = Any amino acid
PFEXLDWEFX

SEQ ID NO: 380 - protein, 10 aa, unidentified
GLLDALDLAS

SEQ ID NO: 381 - protein, 10 aa, unidentified
  VARIANT 3: note = Any amino acid
  VARIANT 10: note = Any amino acid
PEXTAPPEEX

SEQ ID NO: 382 - protein, 10 aa, unidentified
PKTPKKAKKL

SEQ ID NO: 383 - protein, 10 aa, unidentified
ANSRWPTSII

SEQ ID NO: 384 - protein, 10 aa, unidentified
APTYSPPLPP

SEQ ID NO: 385 - protein, 10 aa, unidentified
TSFAEYWNLL

SEQ ID NO: 386 - protein, 11 aa, unidentified
QPPSNPPPRPP

SEQ ID NO: 387 - protein, 11 aa, unidentified
ELLELDKWASL

SEQ ID NO: 388 - protein, 11 aa, unidentified
CTERMALHNLC

SEQ ID NO: 389 - protein, 12 aa, unidentified
  VARIANT 6: note = Any amino acid
PTRVSXPVFGAT

SEQ ID NO: 390 - protein, 12 aa, unidentified
  VARIANT 9: note = Any amino acid
ARTKQTARXSTG

SEQ ID NO: 391 - protein, 12 aa, unidentified
TSFAEYWNLLSP

SEQ ID NO: 392 - protein, 13 aa, unidentified
  VARIANT 13: note = Any amino acid
HSKYPLPPLPSLX

SEQ ID NO: 393 - protein, 13 aa, unidentified
QLTSYDCEVNAPI

SEQ ID NO: 394 - protein, 14 aa, unidentified
  VARIANT 14: note = Any amino acid
ICVWQDWGAHRCTX

SEQ ID NO: 395 - protein, 15 aa, unidentified
LSSLAASSLAKRRQQ

SEQ ID NO: 396 - protein, 11 aa, Alphapapillomavirus 5
  VARIANT 1: note = Any amino acid
XRTRQRNETQV

SEQ ID NO: 397 - protein, 6 aa, Alphapapillomavirus 7
RRETQV

SEQ ID NO: 398 - protein, 11 aa, Alphapapillomavirus 9
RSSRTRRETQV

SEQ ID NO: 399 - protein, 14 aa, unidentified
SISYTDEIEEEDYD

SEQ ID NO: 400 - protein, 7 aa, Bos taurus
  VARIANT 4: note = Any amino acid
DEFXADE

SEQ ID NO: 401 - protein, 13 aa, Conus regius
  VARIANT 13: note = Any amino acid
GCCSDPRCRYRCX

SEQ ID NO: 402 - protein, 9 aa, Dengue virus
FTMRLLSPV

SEQ ID NO: 403 - protein, 7 aa, Drosophila melanogaster
DEVTSTT

SEQ ID NO: 404 - protein, 7 aa, Drosophila melanogaster
PDVSSST

SEQ ID NO: 405 - protein, 11 aa, Drosophila melanogaster
ETNEKNPLPDK

SEQ ID NO: 406 - protein, 15 aa, Drosophila melanogaster
DINNNNNIVEDVERK

SEQ ID NO: 407 - protein, 9 aa, Escherichia coli
  VARIANT 3: note = Any amino acid
HIXDEVAAD

SEQ ID NO: 408 - protein, 13 aa, Escherichia virus T7
LWYTDIRMSWRVP

SEQ ID NO: 409 - protein, 14 aa, Escherichia virus T7
  VARIANT 14: note = Any amino acid
CRVDLQGWRRCRRX

SEQ ID NO: 410 - protein, 11 aa, Gallus gallus
STVHEILSKLS

SEQ ID NO: 411 - protein, 14 aa, Helianthus annuus
GRCYKSRPPICFPN

SEQ ID NO: 412 - protein, 14 aa, Helicobacter pylori
FPLKRHDKVDDLSK

SEQ ID NO: 413 - protein, 8 aa, Hepacivirus C
APPIPPPR

SEQ ID NO: 414 - protein, 9 aa, Hepacivirus C
ALPAWARPD

SEQ ID NO: 415 - protein, 10 aa, Hepacivirus C
KLVALGINAV

SEQ ID NO: 416 - protein, 11 aa, Hepacivirus C
GWLAGLFYQHK

SEQ ID NO: 417 - protein, 15 aa, Hepacivirus C
CQLINTNGSWHINCK

SEQ ID NO: 418 - protein, 7 aa, Hepatitis B virus
  VARIANT 7: note = Any amino acid
PDWDFNX

SEQ ID NO: 419 - protein, 7 aa, Hirudo medicinalis
GDFEEIP

SEQ ID NO: 420 - protein, 9 aa, Hirudo medicinalis
  VARIANT 9: note = Any amino acid
DFEEIPEEX

SEQ ID NO: 421 - protein, 10 aa, Hirudo medicinalis
  VARIANT 9: note = Any amino acid
DFEEIPEEXL

SEQ ID NO: 422 - protein, 10 aa, Hirudo medicinalis
  VARIANT 9: note = Any amino acid
DFEEIPEEXL

SEQ ID NO: 423 - protein, 10 aa, Hirudo medicinalis
  VARIANT 9: note = Any amino acid
DFEEIPEEXL

SEQ ID NO: 424 - protein, 10 aa, Hirudo medicinalis
DFEEIPGEYL

SEQ ID NO: 425 - protein, 10 aa, Hirudo medicinalis
  VARIANT 9: note = Any amino acid
DFEEIPEEXL

SEQ ID NO: 426 - protein, 11 aa, Hirudo medicinalis
  VARIANT 9: note = Any amino acid
DFEEIPEEXLQ

SEQ ID NO: 427 - protein, 5 aa, Homo sapiens
DFEEI

SEQ ID NO: 428 - protein, 5 aa, Homo sapiens
  VARIANT 1: note = Any amino acid
XVPML

SEQ ID NO: 429 - protein, 5 aa, Homo sapiens
CTPSR

SEQ ID NO: 430 - protein, 5 aa, Homo sapiens
MWRPW

SEQ ID NO: 431 - protein, 5 aa, Homo sapiens
CTPSR

SEQ ID NO: 432 - protein, 5 aa, Homo sapiens
  VARIANT 4: note = Any amino acid
KRSXK

SEQ ID NO: 433 - protein, 5 aa, Homo sapiens
VVSHF

SEQ ID NO: 434 - protein, 5 aa, Homo sapiens
ACGRR

SEQ ID NO: 435 - protein, 5 aa, Homo sapiens
  VARIANT 3: note = Any amino acid
ARXSA

SEQ ID NO: 436 - protein, 5 aa, Homo sapiens
  VARIANT 4: note = Any amino acid
QDFXT

SEQ ID NO: 437 - protein, 5 aa, Homo sapiens
  VARIANT 1: note = Any amino acid
XGAGA

SEQ ID NO: 438 - protein, 5 aa, Homo sapiens
YLVTS

SEQ ID NO: 439 - protein, 5 aa, Homo sapiens
  VARIANT 3: note = Any amino acid
NEXTA

SEQ ID NO: 440 - protein, 5 aa, Homo sapiens
GRGDS

SEQ ID NO: 441 - protein, 5 aa, Homo sapiens
ARTKQ

SEQ ID NO: 442 - protein, 5 aa, Homo sapiens
  VARIANT 4: note = Any amino acid
RHRXV

SEQ ID NO: 443 - protein, 5 aa, Homo sapiens
PDYFL

SEQ ID NO: 444 - protein, 5 aa, Homo sapiens
AGAGA

SEQ ID NO: 445 - protein, 5 aa, Homo sapiens
EVYES

SEQ ID NO: 446 - skipped

SEQ ID NO: 447 - protein, 4 aa, Homo sapiens
  SITE 1: note = Acetylated residue
  SITE 4: note = Chloromethylketone modified residue
DEVD

SEQ ID NO: 448 - protein, 5 aa, Homo sapiens
  SITE 5: note = Gamma-amino-butanoic acid
INNNX

SEQ ID NO: 449 - protein, 5 aa, Homo sapiens
EGECY

SEQ ID NO: 450 - protein, 5 aa, Homo sapiens
  SITE 1: note = Acetylated residue
  SITE 2: note = Phosphotyrosine
  SITE 5: note = Amidated residue
TYETL

SEQ ID NO: 451 - protein, 6 aa, Homo sapiens
AVPIAQ

SEQ ID NO: 452 - protein, 6 aa, Homo sapiens
LFSTEV

SEQ ID NO: 453 - protein, 6 aa, Homo sapiens
  VARIANT 4: note = Any amino acid
RQRXAP

SEQ ID NO: 454 - skipped

SEQ ID NO: 455 - protein, 6 aa, Homo sapiens
  VARIANT 4: note = Any amino acid
ARTXQT

SEQ ID NO: 456 - protein, 6 aa, Homo sapiens
  VARIANT 5: note = Any amino acid
QRSTXT

SEQ ID NO: 457 - protein, 6 aa, Homo sapiens
EQVSAV

SEQ ID NO: 458 - protein, 6 aa, Homo sapiens
  VARIANT 3: note = Any amino acid
ARXSCG

SEQ ID NO: 459 - protein, 6 aa, Homo sapiens
  VARIANT 3: note = Any amino acid
SGXVMR

SEQ ID NO: 460 - protein, 6 aa, Homo sapiens
  VARIANT 5: note = Any amino acid
KRRKXV

SEQ ID NO: 461 - protein, 6 aa, Homo sapiens
DAEFRH

SEQ ID NO: 462 - protein, 6 aa, Homo sapiens
  VARIANT 4: note = Any amino acid
ARTXQT

SEQ ID NO: 463 - protein, 6 aa, Homo sapiens
ILHRLL

SEQ ID NO: 464 - protein, 6 aa, Homo sapiens
  VARIANT 4: note = Any amino acid
FSAXPS

SEQ ID NO: 465 - protein, 6 aa, Homo sapiens
GRGDSP

SEQ ID NO: 466 - protein, 6 aa, Homo sapiens
  VARIANT 4: note = Any amino acid
SKSXDR

SEQ ID NO: 467 - protein, 6 aa, Homo sapiens
  VARIANT 5: note = Any amino acid
  VARIANT 6: note = Any amino acid
SSTIXX

SEQ ID NO: 468 - protein, 6 aa, Homo sapiens
  VARIANT 6: note = Any amino acid
TPTPVX

SEQ ID NO: 469 - protein, 6 aa, Homo sapiens
KSRWDE

SEQ ID NO: 470 - protein, 6 aa, Homo sapiens
ILLTPR

SEQ ID NO: 471 - protein, 7 aa, Homo sapiens
PFSKEEC

SEQ ID NO: 472 - protein, 7 aa, Homo sapiens
  VARIANT 4: note = Any amino acid
RSHXYPA

SEQ ID NO: 473 - protein, 7 aa, Homo sapiens
  VARIANT 4: note = Any amino acid
DDIXLLK

SEQ ID NO: 474 - protein, 7 aa, Homo sapiens
  VARIANT 3: note = Any amino acid
NDXIDNP

SEQ ID NO: 475 - protein, 7 aa, Homo sapiens
MDDDFQL

SEQ ID NO: 476 - protein, 7 aa, Homo sapiens
ARTKQTA

SEQ ID NO: 477 - protein, 7 aa, Homo sapiens
ARTKQTA

SEQ ID NO: 478 - protein, 7 aa, Homo sapiens
LPNAIGR

SEQ ID NO: 479 - protein, 7 aa, Homo sapiens
ARTKQTA

SEQ ID NO: 480 - protein, 7 aa, Homo sapiens
GCARSEG

SEQ ID NO: 481 - protein, 7 aa, Homo sapiens
GSRPMYG

SEQ ID NO: 482 - protein, 7 aa, Homo sapiens
  VARIANT 4: note = Any amino acid
ARTXQTA

SEQ ID NO: 483 - protein, 6 aa, Homo sapiens
  SITE 6: note = Amidated residue
VFFAED

SEQ ID NO: 484 - protein, 7 aa, Homo sapiens
RGDINNN

SEQ ID NO: 485 - protein, 8 aa, Homo sapiens
LKFQCGQK

SEQ ID NO: 486 - protein, 8 aa, Homo sapiens
KILHRLLQ

SEQ ID NO: 487 - protein, 8 aa, Homo sapiens
TPLEITEL

SEQ ID NO: 488 - protein, 8 aa, Homo sapiens
  VARIANT 5: note = Any amino acid
  VARIANT 7: note = Any amino acid
YETLXDXE

SEQ ID NO: 489 - protein, 8 aa, Homo sapiens
  VARIANT 4: note = Any amino acid
  VARIANT 8: note = Any amino acid
YAGXDENX

SEQ ID NO: 490 - protein, 8 aa, Homo sapiens
HKILHRLL

SEQ ID NO: 491 - protein, 8 aa, Homo sapiens
GCLHHHRI

SEQ ID NO: 492 - protein, 8 aa, Homo sapiens
MKIPKFTF

SEQ ID NO: 493 - protein, 8 aa, Homo sapiens
  VARIANT 3: note = Any amino acid
ARXSTGGK

SEQ ID NO: 494 - protein, 8 aa, Homo sapiens
KILHRLLQ

SEQ ID NO: 495 - protein, 8 aa, Homo sapiens
AEERIIVL

SEQ ID NO: 496 - protein, 9 aa, Homo sapiens
KILHRLLQD

SEQ ID NO: 497 - protein, 9 aa, Homo sapiens
  SITE 4: note = Phosphotyrosine
EGQYQPQPA

SEQ ID NO: 498 - protein, 9 aa, Homo sapiens
GILGFVFTL

SEQ ID NO: 499 - protein, 9 aa, Homo sapiens
FLWGPRALV

SEQ ID NO: 500 - protein, 9 aa, Homo sapiens
ILGPPGSVY

SEQ ID NO: 501 - protein, 9 aa, Homo sapiens
  VARIANT 4: note = Any amino acid
SDGXTPEPA

SEQ ID NO: 502 - protein, 9 aa, Homo sapiens
KRPPPPPPG

SEQ ID NO: 503 - protein, 9 aa, Homo sapiens
  VARIANT 4: note = Any amino acid
SDGXTPEPA

SEQ ID NO: 504 - protein, 9 aa, Homo sapiens
SIYFPELYD

SEQ ID NO: 505 - protein, 9 aa, Homo sapiens
KILHRLLQE

SEQ ID NO: 506 - protein, 9 aa, Homo sapiens
  VARIANT 3: note = Any amino acid
SPXLPNITL

SEQ ID NO: 507 - protein, 9 aa, Homo sapiens
EPEEPEADQ

SEQ ID NO: 508 - protein, 9 aa, Homo sapiens
HKILHRLLQ

SEQ ID NO: 509 - protein, 9 aa, Homo sapiens
  VARIANT 4: note = Any amino acid
RQAXLSISV

SEQ ID NO: 510 - protein, 9 aa, Homo sapiens
  VARIANT 5: note = Any amino acid
  VARIANT 9: note = Any amino acid
AYDEXDEEX

SEQ ID NO: 511 - protein, 9 aa, Homo sapiens
EAIIRKALM

SEQ ID NO: 512 - protein, 10 aa, Homo sapiens
  VARIANT 5: note = Any amino acid
PQPEXVNQPD

SEQ ID NO: 513 - protein, 10 aa, Homo sapiens
DFGGFEAAET

SEQ ID NO: 514 - protein, 10 aa, Homo sapiens
VPPPVPPPPS

SEQ ID NO: 515 - protein, 10 aa, Homo sapiens
GAFQNLFQSV

SEQ ID NO: 516 - protein, 10 aa, Homo sapiens
PGRQACGRRH

SEQ ID NO: 517 - protein, 10 aa, Homo sapiens
  VARIANT 4: note = Any amino acid
ARTXQTARKS

SEQ ID NO: 518 - protein, 10 aa, Homo sapiens
RHKILHRLLQ

SEQ ID NO: 519 - protein, 10 aa, Homo sapiens
  VARIANT 4: note = Any amino acid
LQRXSSDPTG

SEQ ID NO: 520 - protein, 10 aa, Homo sapiens
SYEGYEGYYS

SEQ ID NO: 521 - protein, 10 aa, Homo sapiens
GMHSKGTQTA

SEQ ID NO: 522 - protein, 10 aa, Homo sapiens
DVPQWEVFFK

SEQ ID NO: 523 - protein, 10 aa, Homo sapiens
GSYGHLSIGT

SEQ ID NO: 524 - protein, 10 aa, Homo sapiens
ETGTTNTATT

SEQ ID NO: 525 - protein, 10 aa, Homo sapiens
KSLLQQLLTE

SEQ ID NO: 526 - protein, 10 aa, Homo sapiens
LTGCGDIIAE

SEQ ID NO: 527 - protein, 10 aa, Homo sapiens
HNWGQGFRLG

SEQ ID NO: 528 - protein, 10 aa, Homo sapiens
RNQKRKHDEI

SEQ ID NO: 529 - protein, 10 aa, Homo sapiens
KILHRLLQDS

SEQ ID NO: 530 - protein, 10 aa, Homo sapiens
ENLLLRYLLD

SEQ ID NO: 531 - protein, 11 aa, Homo sapiens
SHRPPPPGHRV

SEQ ID NO: 532 - protein, 11 aa, Homo sapiens
KPQPAVPPRPS

SEQ ID NO: 533 - protein, 11 aa, Homo sapiens
DEDVKDNWDDD

SEQ ID NO: 534 - protein, 11 aa, Homo sapiens
RPKPQQFFGLM

SEQ ID NO: 535 - protein, 11 aa, Homo sapiens
NALLRYLLDKD

SEQ ID NO: 536 - protein, 11 aa, Homo sapiens
EPIAIIITDTE

SEQ ID NO: 537 - protein, 11 aa, Homo sapiens
  VARIANT 10: note = Any amino acid
EAEERIIVLXD

SEQ ID NO: 538 - protein, 11 aa, Homo sapiens
YQGGGEEMALP

SEQ ID NO: 539 - protein, 11 aa, Homo sapiens
GMRPPPPGIRG

SEQ ID NO: 540 - protein, 12 aa, Homo sapiens
  VARIANT 7: note = Any amino acid
STSRHKXLMFKT

SEQ ID NO: 541 - protein, 12 aa, Homo sapiens
PPVPNPDYEPIR

SEQ ID NO: 542 - protein, 12 aa, Homo sapiens
AYMRADAAAGGA

SEQ ID NO: 543 - protein, 12 aa, Homo sapiens
KENALLRYLLDK

SEQ ID NO: 544 - protein, 12 aa, Homo sapiens
QPKDDKNTMTDS

SEQ ID NO: 545 - protein, 12 aa, Homo sapiens
DHQLLRYLLDKD

SEQ ID NO: 546 - protein, 12 aa, Homo sapiens
AADMEEYEESGL

SEQ ID NO: 547 - protein, 12 aa, Homo sapiens
ANALLRYLLDKD

SEQ ID NO: 548 - protein, 12 aa, Homo sapiens
PSLLKKLLLAPA

SEQ ID NO: 549 - protein, 12 aa, Homo sapiens
  VARIANT 4: note = Any amino acid
  VARIANT 11: note = Any amino acid
  VARIANT 12: note = Any amino acid
ARTXQTARKSXX

SEQ ID NO: 550 - protein, 12 aa, Homo sapiens
FSLDDLQPWHSF

SEQ ID NO: 551 - protein, 13 aa, Homo sapiens
SDWRFLRGYHQYA

SEQ ID NO: 552 - protein, 13 aa, Homo sapiens
PATLKICSWNVDG

SEQ ID NO: 553 - protein, 13 aa, Homo sapiens
PSKKRKRSRWNQD

SEQ ID NO: 554 - protein, 13 aa, Homo sapiens
GGGEQLAINELIS

SEQ ID NO: 555 - protein, 13 aa, Homo sapiens
  VARIANT 13: note = Any amino acid
NPPPPGFIMHGNX

SEQ ID NO: 556 - protein, 13 aa, Homo sapiens
SRGLEVSHRLAPW

SEQ ID NO: 557 - protein, 13 aa, Homo sapiens
EDDDIKQLAAWAT

SEQ ID NO: 558 - protein, 13 aa, Homo sapiens
SGPWKPAKPAPSV

SEQ ID NO: 559 - protein, 13 aa, Homo sapiens
KWTLERLKRKYRN

SEQ ID NO: 560 - protein, 14 aa, Homo sapiens
LLPTPPLSPSRRSG

SEQ ID NO: 561 - protein, 14 aa, Homo sapiens
EDHKPGTFPKALTN

SEQ ID NO: 562 - protein, 14 aa, Homo sapiens
  VARIANT 4: note = Any amino acid
  VARIANT 9: note = Any amino acid
ARTXQTARXSTGGK

SEQ ID NO: 563 - protein, 14 aa, Homo sapiens
LNKYQGFIFDIVTK

SEQ ID NO: 564 - protein, 14 aa, Homo sapiens
NMGLEAIIRKALMG

SEQ ID NO: 565 - protein, 14 aa, Homo sapiens
EGVLANFFNSLLSK

SEQ ID NO: 566 - protein, 15 aa, Homo sapiens
GELIGILNAAKVPAD

SEQ ID NO: 567 - protein, 15 aa, Homo sapiens
  VARIANT 9: note = Any amino acid
ARTKQTARXSTGGKA

SEQ ID NO: 568 - protein, 15 aa, Homo sapiens
FKEVANAVKISASLM

SEQ ID NO: 569 - protein, 15 aa, Homo sapiens
SQETFSDLWKLLPEN

SEQ ID NO: 570 - protein, 15 aa, Homo sapiens
IRNKIRAIGKMARVF

SEQ ID NO: 571 - protein, 15 aa, Homo sapiens
WPPEFHPGVPWKGLQ

SEQ ID NO: 572 - protein, 15 aa, Homo sapiens
QGITKPAIRRLARRG

SEQ ID NO: 573 - protein, 15 aa, Homo sapiens
  VARIANT 9: note = Any amino acid
APPAYEKLXAEQSPP

SEQ ID NO: 574 - protein, 15 aa, Homo sapiens
TLEKLMKAFESLKSF

SEQ ID NO: 575 - protein, 15 aa, Homo sapiens
GNTKNHPMLMNLLKD

SEQ ID NO: 576 - protein, 10 aa, Human gammaherpesvirus 4
APQPAPENAY

SEQ ID NO: 577 - protein, 9 aa, Human immunodeficiency virus
ISPRTLDAW

SEQ ID NO: 578 - protein, 7 aa, Human immunodeficiency virus 1
ELDKWAS

SEQ ID NO: 579 - protein, 8 aa, Human immunodeficiency virus 1
VPLRPMTY

SEQ ID NO: 580 - protein, 8 aa, Human immunodeficiency virus 1
GGRKKYKL

SEQ ID NO: 581 - protein, 12 aa, Human immunodeficiency virus 1
  VARIANT 11: note = Any amino acid
VHAGPIAPGQXR

SEQ ID NO: 582 - protein, 9 aa, Human mastadenovirus C
PPTLHELYD

SEQ ID NO: 583 - protein, 9 aa, Human papillomavirus
DLYCYEQLN

SEQ ID NO: 584 - protein, 5 aa, Influenza A virus
ARTAR

SEQ ID NO: 585 - protein, 8 aa, Influenza A virus
  VARIANT 6: note = Any amino acid
  VARIANT 8: note = Any amino acid
RTARSXVX

SEQ ID NO: 586 - protein, 9 aa, Influenza A virus
  SITE 7: note = N-methyl-L-alanine
FVKQNAAAL

SEQ ID NO: 587 - protein, 11 aa, Mesocricetus auratus polyomavirus 1
  VARIANT 4: note = Any amino acid
EPQXEEIPIYL

SEQ ID NO: 588 - skipped

SEQ ID NO: 589 - protein, 6 aa, Mus musculus
EVCPAG

SEQ ID NO: 590 - protein, 6 aa, Mus musculus
ESSDSQ

SEQ ID NO: 591 - protein, 7 aa, Mus musculus
DDWTHLS

SEQ ID NO: 592 - protein, 10 aa, Mus musculus
HKILHRLLQD

SEQ ID NO: 593 - protein, 11 aa, Mus musculus
HKILHRLLQEG

SEQ ID NO: 594 - protein, 12 aa, Mus musculus
KKRLLSFRDVDF

SEQ ID NO: 595 - protein, 15 aa, Mus musculus
GTYGVFTNAAYDPTP

SEQ ID NO: 596 - protein, 11 aa, synthetic construct
  SITE 1: note = N-methylcarbonylthreonine
  SITE 4: note = (2R)-2-amino-2-methylnonanoic acid
  SITE 7: note = 6-chloro-L-tryptophan
  SITE 11: note = Amidated 2-methyl-L-norleucine
TSFXEYXALLX

SEQ ID NO: 597 - protein, 13 aa, unidentified
SDFEEFSLDDIEQ

SEQ ID NO: 598 - protein, 9 aa, Primate T-lymphotropic virus 1
LLFGYPVYV

SEQ ID NO: 599 - protein, 9 aa, Primate T-lymphotropic virus 1
LLFGYPVYV

SEQ ID NO: 600 - protein, 13 aa, Rabies lyssavirus
SWESHKSGGETRL

SEQ ID NO: 601 - protein, 9 aa, Rattus norvegicus
  VARIANT 7: note = Any amino acid
RAVENQXSF

SEQ ID NO: 602 - protein, 9 aa, Rattus norvegicus
SFGDGFADF

SEQ ID NO: 603 - protein, 15 aa, Rattus norvegicus
EVCAIKIQRAYRRHL

SEQ ID NO: 604-606 - skipped (3 entries)

SEQ ID NO: 607 - protein, 6 aa, synthetic construct
ILHRLL

SEQ ID NO: 608 - skipped

SEQ ID NO: 609 - protein, 7 aa, synthetic construct
SWFQTDL

SEQ ID NO: 610 - protein, 8 aa, synthetic construct
KILHRLLQ

SEQ ID NO: 611 - protein, 9 aa, synthetic construct
  VARIANT 4: note = Any amino acid
TARXSTGGW

SEQ ID NO: 612 - protein, 9 aa, synthetic construct
  SITE 9: note = {[(2R)-2,3-diamino-3-oxopropyl]sulfanyl}acetic acid
KFEGYDNEX

SEQ ID NO: 613 - protein, 10 aa, synthetic construct
CPAYSRYLDC

SEQ ID NO: 614 - protein, 11 aa, synthetic construct
  VARIANT 9: note = Any amino acid
ARTKQTARXST

SEQ ID NO: 615 - protein, 10 aa, synthetic construct
  DISULFID 1..10: note = Intrachain Disulfide Bond
CPARFAALFC

SEQ ID NO: 616 - protein, 14 aa, synthetic construct
SKYITTIAGVMTLS

SEQ ID NO: 617 - protein, 15 aa, synthetic construct
CWVLEYDMFGALHCR

SEQ ID NO: 618-619 - skipped (2 entries)

SEQ ID NO: 620 - protein, 5 aa, Xenopus tropicalis
ARTKQ

SEQ ID NO: 621 - protein, 86 aa, synthetic construct
MIKEEHVIIQAEFYLNPDQSGEFMFDFDGDEIFHVDMAKKETVWRLEEFGRFASFEAQGALANIAVDKANLEIMTKRSNYTPITNV

SEQ ID NO: 622 - protein, 32 aa, synthetic construct
QSEAGSHTVQRMYGCDVGSDWRFLRGYHQYAY

SEQ ID NO: 623 - protein, 183 aa, synthetic construct
MIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDA

SEQ ID NO: 624 - protein, 47 aa, synthetic construct
GSHSMRYFFTSVSRPGRGEPRFIAVGYVDDTQFVRFDSDAASQRMEP

SEQ ID NO: 625 - protein, 140 aa, synthetic construct
ADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWE

SEQ ID NO: 626 - protein, 189 aa, synthetic construct
QSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWE

SEQ ID NO: 627 - protein, 94 aa, synthetic construct
MGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSH

SEQ ID NO: 628 - protein, 55 aa, synthetic construct
SDVGEYRAVTELGRPDAEYWNSQKDLLEQRRAAVDTYCRHNYGVGESFTVQRRVE

SEQ ID NO: 629 - protein, 96 aa, synthetic construct
GDTRPRFLWQLKFECHFFNGTERVRLLERCIYNQEESVRFDSDVGEYRAVTELGRPDAEYWNSQKDLLEQRRAAVDTYCRHNYGVGESFTVQRRVE

SEQ ID NO: 630 - protein, 97 aa, synthetic construct
MGDTRPRFLWQLKFECHFFNGTERVRLLERCIYNQEESVRFDSDVGEYRAVTELGRPDAEYWNSQKDLLEQRRAAVDTYCRHNYGVGESFTVQRRVE

SEQ ID NO: 631 - protein, 97 aa, synthetic construct; identical to SEQ ID NO: 630

SEQ ID NO: 632 - protein, 97 aa, synthetic construct; identical to SEQ ID NO: 630

SEQ ID NO: 633 - protein, 190 aa, synthetic construct
GDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRA

SEQ ID NO: 634 - protein, 49 aa, synthetic construct
GDTRPRFLWQLKFECHFFNGTERVRLLERCIYNQEESVRFDSDVGEYRA

SEQ ID NO: 635 - protein, 190 aa, synthetic construct; identical to SEQ ID NO: 633

SEQ ID NO: 636 - protein, 173 aa, synthetic construct
MIKEEHVIIQAEFYLNPDQSGEFMFDFDGDEIFHVDMAKKETVWRLEEFGRFASFEAQGALANIAVDKANLEIMTKRSNYTPITNVQSEAGSHTVQRMYGCDVGSDWRFLRGYHQYAYSDVGEYRAVTELGRPDAEYWNSQKDLLEQRRAAVDTYCRHNYGVGESFTVQRRVE

SEQ ID NO: 637 - protein, 197 aa, synthetic construct
MIKEEHVIIQAEFYLNPDQSGEFMFDFDGDEIFHVDMAKKETVWRLEEFGRFASFEAQGALANIAVDKANLEIMTKRSNYTPITNVGGGGSGGGGSGGGGSGDTRPRFLWQLKFECHFFNGTERVRLLERCIYNQEESVRFDSDVGEYRAVTELGRPDAEYWNSQKDLLEQRRAAVDTYCRHNYGVGESFTVQRRVE

SEQ ID NO: 638 - protein, 190 aa, synthetic construct
MIKEEHVIIQAEFYLNPDQSGEFMFDFDGDEIFHVDMAKKETVWRLEEFGRFASFEAQGALANIAVDKANLEIMTKRSNYTPITNVGGGGGGGGGDTRPRFLWQLKFECHFFNGTERVRLLERCIYNQEESVRFDSDVGEYRAVTELGRPDAEYWNSQKDLLEQRRAAVDTYCRHNYGVGESFTVQRRVE

SEQ ID NO: 639 - protein, 188 aa, synthetic construct
MIKEEHVIIQAEFYLNPDQSGEFMFDFDGDEIFHVDMAKKETVWRLEEFGRFASFEAQGALANIAVDKANLEIMTKRSNYTPITNVGGGGGGGDTRPRFLWQLKFECHFFNGTERVRLLERCIYNQEESVRFDSDVGEYRAVTELGRPDAEYWNSQKDLLEQRRAAVDTYCRHNYGVGESFTVQRRVE

SEQ ID NO: 640 - protein, 187 aa, synthetic construct
MIKEEHVIIQAEFYLNPDQSGEFMFDFDGDEIFHVDMAKKETVWRLEEFGRFASFEAQGALANIAVDKANLEIMTKRSNYTPITNVGGGGSGDTRPRFLWQLKFECHFFNGTERVRLLERCIYNQEESVRFDSDVGEYRAVTELGRPDAEYWNSQKDLLEQRRAAVDTYCRHNYGVGESFTVQRRVE

SEQ ID NO: 641 - protein, 184 aa, synthetic construct
MIKEEHVIIQAEFYLNPDQSGEFMFDFDGDEIFHVDMAKKETVWRLEEFGRFASFEAQGALANIAVDKANLEIMTKRSNYTPITNVLEGDTRPRFLWQLKFECHFFNGTERVRLLERCIYNQEESVRFDSDVGEYRAVTELGRPDAEYWNSQKDLLEQRRAAVDTYCRHNYGVGESFTVQRRVE

SEQ ID NO: 642 - protein, 192 aa, synthetic construct
MIKEEHVIIQAEFYLNPDQSGEFMFDFDGDEIFHVDMAKKETVWRLEEFGRFASFEAQGALANIAVDKANLEIMTKRSNYTPITNVGGGGSGGGGSGDTRPRFLWQLKFECHFFNGTERVRLLERCIYNQEESVRFDSDVGEYRAVTELGRPDAEYWNSQKDLLEQRRAAVDTYCRHNYGVGESFTVQRRVE

SEQ ID NO: 643 - protein, 185 aa, synthetic construct
MGDTRPRFLWQLKFECHFFNGTERVRLLERCIYNQEESVRFDSDVGEYRAVTELGRPDAEYWNSQKDLLEQRRAAVDTYCRHNYGVGESFTVQRRVELEMIKEEHVIIQAEFYLNPDQSGEFMFDFDGDEIFHVDMAKKETVWRLEEFGRFASFEAQGALANIAVDKANLEIMTKRSNYTPITNV

SEQ ID NO: 644 - protein, 190 aa, synthetic construct
MGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEQSEAGSHMIKEEHVIIQAEFYLNPDQSGEFMFDFDGDEIFHVDMAKKETVWRLEEFGRFASFEAQGALANIAVDKANLEIMTKRSNYTPITNV

SEQ ID NO: 645 - protein, 287 aa, synthetic construct
MGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEQSEAGSHMIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDA

SEQ ID NO: 646 - protein, 278 aa, synthetic construct
MIKEEHVIIQAEFYLNPDQSGEFMFDFDGDEIFHVDMAKKETVWRLEEFGRFASFEAQGALANIAVDKANLEIMTKRSNYTPITNVLEGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRA

SEQ ID NO: 647 - protein, 252 aa, synthetic construct
MIKEEHVIIQAEFYLNPDQSGEFMFDFDGDEIFHVDMAKKETVWRLEEFGRFASFEAQGALANIAVDKANLEIMTKRSNYTPITNVLEGDTRPRFLWQLKFECHFFNGTERVRLLERCIYNQEESVRFDSDVGEYRAVTELGRPDAEYWNSQKDLLEQRRAAVDTYCRHNYGVGESFTVQRRVESSGMKAIFVLNAQHDEAVDANSLAEAKVLANRELDKYGVSDYYKNLINNAKTVEGVKALIDEILAALP

SEQ ID NO: 648 - protein, 280 aa, synthetic construct
GSHSMRYFFTSVSRPGRGEPRFIAVGYVDDTQFVRFDSDAASQRMEPRLEEFGRFASFEAQGALANIAVDKANLEIMTKRSNYTPITNVLEGDTRPRFLWQLKFECHFFNGTERVRLLERCIYNQEESVRFDSDVGEYRAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWE

SEQ ID NO: 649 - protein, 286 aa, synthetic construct
MGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWE

SEQ ID NO: 650 - protein, 284 aa, synthetic construct
MGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRA

SEQ ID NO: 651 - protein, 188 aa, synthetic construct
MIKEEHVIIQAEFYLNPDQSGEFMFDFDGDEIFHVDMAKKETVWRLEEFGRFASFEAQGALANIAVDKANLEIMTKRSNYTPLEMGDTRPRFLWQLKFECHFFNGTERVRLLERCIYNQEESVRFDSDVGEYRAVTELGRPDAEYWNSQKDLLEQRRAAVDTYCRHNYGVGESFTVQGGSHHHHHHLE

SEQ ID NO: 652 - protein, 189 aa, synthetic construct
MIKEEHVIIQAEFYLNPDQSGEFMFDFDGDEIFHVDMAKKETVWRLEEFGRFASFEAQGALANIAVDKANLEIMTKRSNYTPGGSMGDTRPRFLWQLKFECHFFNGTERVRLLERCIYNQEESVRFDSDVGEYRAVTELGRPDAEYWNSQKDLLEQRRAAVDTYCRHNYGVGESFTVQGGSHHHHHHLE

SEQ ID NO: 653 - protein, 189 aa, synthetic construct
MIKEEHVIIQAEFYLNPDQSGEFMFDFDGDEIFHVDMAKKETVWRLEEFGRFASFEAQGALANIAVDKANLEIMTKRSNYTPSSGMGDTRPRFLWQLKFECHFFNGTERVRLLERCIYNQEESVRFDSDVGEYRAVTELGRPDAEYWNSQKDLLEQRRAAVDTYCRHNYGVGESFTVQGGSHHHHHHLE

SEQ ID NO: 654 - protein, 191 aa, synthetic construct
MIKEEHVIIQAEFYLNPDQSGEFMFDFDGDEIFHVDMAKKETVWRLEEFGRFASFEAQGALANIAVDKANLEIMTKRSNYTPGGGGSMGDTRPRFLWQLKFECHFFNGTERVRLLERCIYNQEESVRFDSDVGEYRAVTELGRPDAEYWNSQKDLLEQRRAAVDTYCRHNYGVGESFTVQGGSHHHHHHLE

SEQ ID NO: 655 - protein, 186 aa, synthetic construct
MIKEEHVIIQAEFYLNPDQSGEFMFDFDGDEIFHVDMAKKETVWRLEEFGRFASFEAQGALANIAVDKANLEIMTKRSNYTPMGDTRPRFLWQLKFECHFFNGTERVRLLERCIYNQEESVRFDSDVGEYRAVTELGRPDAEYWNSQKDLLEQRRAAVDTYCRHNYGVGESFTVQGGSHHHHHHLE

SEQ ID NO: 656 - protein, 203 aa, synthetic construct
MIKEEHVIIQAEFYLNPDQSGEFMFDFDGDEIFHVDMAKKETVWRLEEFGRFASFEAQGALANIAVDKANLEIMTKRSNYTPPVSKMRMATPLLMQALPMGDTRPRFLWQLKFECHFFNGTERVRLLERCIYNQEESVRFDSDVGEYRAVTELGRPDAEYWNSQKDLLEQRRAAVDTYCRHNYGVGESFTVQGGSHHHHHHLE

SEQ ID NO: 657 - protein, 289 aa, synthetic construct
MIKEEHVIIQAEFYLNPDQSGEFMFDFDGDEIFHVDMAKKETVWRLEEFGRFASFEAQGALANIAVDKANLEIMTKRSNYTPMGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGSHHHHHHLE

SEQ ID NO: 658 - protein, 256 aa, synthetic construct
MIKEEHVIIQAEFYLNPDQSGEFMFDFDGDEIFHVDMAKKETVWRLEEFGRFASFEAQGALANIAVDKANLEIMTKRSNYTPLEMGDTRPRFLWQLKFECHFFNGTERVRLLERCIYNQEESVRFDSDVGEYRAVTELGRPDAEYWNSQKDLLEQRRAAVDTYCRHNYGVGESFTVQGGSMKAIFVLNAQHDEAVDANSLAEAKVLANRELDKYGVSDYYKNLINNAKTVEGVKALIDEILAALPGGSHHHHHHLE

SEQ ID NO: 659 - protein, 249 aa, synthetic construct
MIKEEHVIIQAEFYLNPDQSGEFMFDFDGDEIFHVDMAKKETVWRLEEFGRFASFEAQGALANIAVDKANLEIMTKRSNYTPLEMGDTRPRFLWQLKFECHFFNGTERVRLLERCIYNQEESVRFDSDVGEYRAVTELGRPDAEYWNSQKDLLEQRRAAVDTYCRHNYGVGESFTVQGGSKTKNVVCNEELTELAEAKVLANRELDKYGVSDYHKNLINNAKTVEGVKDLQAQVVESAGGSHHHHHHLE

SEQ ID NO: 660 - protein, 253 aa, synthetic construct
MIKEEHVIIQAEFYLNPDQSGEFMFDFDGDEIFHVDMAKKETVWRLEEFGRFASFEAQGALANIAVDKANLEIMTKRSNYTPLEMGDTRPRFLWQLKFECHFFNGTERVRLLERCIYNQEESVRFDSDVGEYRAVTELGRPDAEYWNSQKDLLEQRRAAVDTYCRHNYGVGESFTVQGGSKTKNVVCNEELTALAKAKVLANRELDKYGVSDYYKKLVNKAKAAETVATLQAQVVESAKKARGGSHHHHHHLE

SEQ ID NO: 661 - protein, 260 aa, synthetic construct
MIKEEHVIIQAEFYLNPDQSGEFMFDFDGDEIFHVDMAKKETVWRLEEFGRFASFEAQGALANIAVDKANLEIMTKRSNYTPLEMGDTRPRFLWQLKFECHFFNGTERVRLLERCIYNQEESVRFDSDVGEYRAVTELGRPDAEYWNSQKDLLEQRRAAVDTYCRHNYGVGESFTVQGGSMKAIFVLNAQHDEAVDANSALAKAKVLANRELDKYGVSDYYKKLVNNAKTVEGVKDLQAQVVESAKKARGGSHHHHHHLE

SEQ ID NO: 662 - protein, 366 aa, synthetic construct
MIKEEHVIIQAEFYLNPDQSGEFMFDFDGDEIFHVDMAKKETVWRLEEFGRFASFEAQGALANIAVDKANLEIMTKRSNYTPMGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGSMKAIFVLNAQHDEAVDANSLAEAKVLANRELDKYGVSDYYKNLINNAKTVEGVKALIDEILAALPGGSENLYFQGGSHHHHHHLE

SEQ ID NO: 663 - protein, 361 aa, synthetic construct
MIKEEHVIIQAEFYLNPDQSGEFMFDFDGDEIFHVDMAKKETVWRLEEFGRFASFEAQGALANIAVDKANLEIMTKRSNYTPMGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGSMKAIFVLNAQHDEAVDANSALAKAKVLANRELDKYGVSDYYKKLVNNAKTVEGVKDLQAQVVESAKKARGGSHHHHHHLE

SEQ ID NO: 664 - protein, 357 aa, synthetic construct
MIKEEHVIIQAEFYLNPDQSGEFMFDFDGDEIFHVDMAKKETVWRLEEFGRFASFEAQGALANIAVDKANLEIMTKRSNYTPMGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPMKAIFVLNAQHDEAVDANSLAEAKVLANRELDKYGVSDYYKNLINNAKTVEGVKALIDEILAALPGGSQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGSHHHHHHLE

SEQ ID NO: 665 - protein, 361 aa, synthetic construct
MIKEEHVIIQAEFYLNPDQSGEFMFDFDGDEIFHVDMAKKETVWRLEEFGRFASFEAQGALANIAVDKANLEIMTKRSNYTPMGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPMKAIFVLNAQHDEAVDANSALAKAKVLANRELDKYGVSDYYKKLVNNAKTVEGVKDLQAQVVESAKKARGGSQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGSHHHHHHLE

SEQ ID NO: 666 - protein, 357 aa, synthetic construct
MKAIFVLNAQHDEAVDANSLAEAKVLANRELDKYGVSDYYKNLINNAKTVEGVKALIDEILAALPGGSMIKEEHVIIQAEFYLNPDQSGEFMFDFDGDEIFHVDMAKKETVWRLEEFGRFASFEAQGALANIAVDKANLEIMTKRSNYTPMGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGSHHHHHHLE

SEQ ID NO: 667 - protein, 361 aa, synthetic construct
MKAIFVLNAQHDEAVDANSALAKAKVLANRELDKYGVSDYYKKLVNNAKTVEGVKDLQAQVVESAKKARGGSMIKEEHVIIQAEFYLNPDQSGEFMFDFDGDEIFHVDMAKKETVWRLEEFGRFASFEAQGALANIAVDKANLEIMTKRSNYTPMGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGSHHHHHHLE

SEQ ID NO: 668 - protein, 360 aa, synthetic construct
MIKEEHVIIQAEFYLNPDQSGEFMFDFDGDEIFHVDMAKKETVWRLEEFGRFASFEAQGALANIAVDKANLEIMTKRSNYTPMGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKGGSMKAIFVLNAQHDEAVDANSLAEAKVLANRELDKYGVSDYYKNLINNAKTVEGVKALIDEILAALPGGSHHHHHHLE

SEQ ID NO: 669 - protein, 364 aa, synthetic construct
MIKEEHVIIQAEFYLNPDQSGEFMFDFDGDEIFHVDMAKKETVWRLEEFGRFASFEAQGALANIAVDKANLEIMTKRSNYTPMGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKGGSMKAIFVLNAQHDEAVDANSALAKAKVLANRELDKYGVSDYYKKLVNNAKTVEGVKDLQAQVVESAKKARGGSHHHHHHLE

SEQ ID NO: 670 - protein, 361 aa, synthetic construct
MIKEEHVIIQAEFYLNPDQSGEFMFDFDGDEIFHVDMAKKETVWRLEEFGRFASFEAQGALANIAVDKANLEIMTKRSNYTPLEMGDTRPRFLWQLKFECHFFNGTERVRLLERCIYNQEESVRFDSDVGEYRAVTELGRPDAEYWNSQKDLLEQRRAAVDTYCRHNYGVGESFTVQGGSMKAIFVLNAQHDEAVDANSALAKAKVLANRELDKYGVSDYYKKLVNNAKTVEGVKDLQAQVVESAKKARGGSQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGSHHHHHHLE

SEQ ID NO: 671 - protein, 361 aa, synthetic construct
MIKEEHVIIQAEFYLNPDQSGEFMFDFDGDEIFHVDMAKKETVWRLEEFGRFASFEAQGALANIAVDKANLEIMTKRSNYTPLEMGDTRPRFLWQLKFECHFFNGTERVRLLERCIYNQEESVRFDSDVGEYRAVTELGRPDAEYWNSQKDLLEQRRAAVDTYCRHNYGVGESFTVQGGSQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGSMKAIFVLNAQHDEAVDANSALAKAKVLANRELDKYGVSDYYKKLVNNAKTVEGVKDLQAQVVESAKKARGGSHHHHHHLE

SEQ ID NO: 672 - protein, 361 aa, synthetic construct
MKAIFVLNAQHDEAVDANSALAKAKVLANRELDKYGVSDYYKKLVNNAKTVEGVKDLQAQVVESAKKARGGSMIKEEHVIIQAEFYLNPDQSGEFMFDFDGDEIFHVDMAKKETVWRLEEFGRFASFEAQGALANIAVDKANLEIMTKRSNYTPLEMGDTRPRFLWQLKFECHFFNGTERVRLLERCIYNQEESVRFDSDVGEYRAVTELGRPDAEYWNSQKDLLEQRRAAVDTYCRHNYGVGESFTVQGGSQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGSHHHHHHLE

SEQ ID NO: 673 - protein, 286 aa, synthetic construct
MSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGMIKEEHVIIQAEFYLNPDQSGEFMFDFDGDEIFHVDMAKKETVWRLEEFGRFASFEAQGALANIAVDKANLEIMTKRSNYTPLEMGDTRPRFLWQLKFECHFFNGTERVRLLERCIYNQEESVRFDSDVGEYRAVTELGRPDAEYWNSQKDLLEQRRAAVDTYCRHNYGVGESFTVQGGSHHHHHHLE

SEQ ID NO: 674 - protein, 200 aa, synthetic construct
MSEDDEEKEEDGMIKEEHVIIQAEFYLNPDQSGEFMFDFDGDEIFHVDMAKKETVWRLEEFGRFASFEAQGALANIAVDKANLEIMTKRSNYTPLEMGDTRPRFLWQLKFECHFFNGTERVRLLERCIYNQEESVRFDSDVGEYRAVTELGRPDAEYWNSQKDLLEQRRAAVDTYCRHNYGVGESFTVQGGSHHHHHHLE

SEQ ID NO: 675 - protein, 361 aa, synthetic construct
MSEDDEEKEEDGLVGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTAGGSHHHHHH

SEQ ID NO: 676 - protein, 348 aa, synthetic construct
MGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTAGGSHHHHHH

SEQ ID NO: 677 - protein, 193 aa, synthetic construct
MGSHSMRYFYTSVSRPGRGEPRFISVGYVDDDQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADGGSHHHHHH

SEQ ID NO: 678 - protein, 193 aa, synthetic construct
MGSHSMRYFYTSVSRPGRGEPRFISVGYVDGDQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADGGSHHHHHH

SEQ ID NO: 679 - protein, 193 aa, synthetic construct
MGSHSMRYFYTSVSRPGRGEPRFISVGYVDGDEIFHVDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADGGSHHHHHH

SEQ ID NO: 680 - protein, 193 aa, synthetic construct
MGSHSMRYFYTSVSRPGRGEPRFISVGYVDGDEIFHVDSDAASPREEPRAPWIEQEGPSYGDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADGGSHHHHHH

SEQ ID NO: 681 - protein, 193 aa, synthetic construct
MGSHSMRYFYTSVSRPGRGEPRFISVGYVDGDEIFHVDSDAASPREEPRAPWIEQEGPSYGDRNTQIYKAQAQTDRESLRNLRGYYNQSEAGSHTLQSMYGCDVGPDGRLLRGHDQYAYDGKDYIALNEDLRSWTAADTAAQITQRKWETNREAEQRRAYLEGECVEWLRRYLENGKDKLERADGGSHHHHHH

SEQ ID NO: 682 - protein, 193 aa, synthetic construct
MGSHSMRYFYTSVSRPGRGEPRFISVGYVDGDEIFHVDSDAASPREEPRAPWIEQEGPSYGDRNTQIYKAQAQTDRESLRNLRGYYNQSEAGSHTLQSMYGCDVGPDGRLLRGHDQYAYDGKDYIALNEDLRSWTAADTAAQITQRKWETQKEAEQRRAYLEGECVEWLRRYLENGKDKLERADGGSHHHHHH

SEQ ID NO: 683 - protein, 193 aa, synthetic construct
MGSHSMRYFYTSVSRPGRGEPRFISVGYVDGDEIFHVDSDAASPREEPRAPWIEQEGPSYGDRNTQIYKAQAQTDRESLRNLRGYYNQSEAGSHTLQSMYGCDVGPDGRLLRGHDQYAYDGKDYIALNEDLRSWTAADTAAQITQRKWETQKDLLQRRAYLEGECVEWLRRYLENGKDKLERADGGSHHHHHH

SEQ ID NO: 684 - protein, 193 aa, synthetic construct
MGSHSMRYFYTSVSRPGRGEPRFISVGYVDGDEIFHVDSDAASPREEPRLEEFEQEGPSYGDRNTQIYKAQAQTDRESLRNLRGYYNQSEAGSHTLQSMYGCDVGPDGRLLRGHDQYAYDGKDYIALNEDLRSWTAADTAAQITQRKWETQKEAEQRRAYLEGECVEWLRRYLENGKDKLERADGGSHHHHHH

SEQ ID NO: 685 - protein, 370 aa, synthetic construct
MGSHSMRYFYTSVSRPGRGEPRFISVGYVDGDEIFHVDSDAASPREEPRLEEFEQEGPSYGDRNTQIYKAQAQTDRESLRNLRGYYNQSEAGSHTLQSMYGCDVGPDGRLLRGHDQYAYDGKDYIALNEDLRSWTAADTAAQITQRKWETQKEAEQRRAYLEGECVEWLRRYLENGKDKLERADQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGSMKAIFVLNAQHDEAVDANSLAEAKVLANRELDKYGVSDYYKNLINNAKTVEGVKALIDEILAALPGGSENLYFQGGSHHHHHHLE

SEQ ID NO: 686 - protein, 293 aa, synthetic construct
MGSHSMRYFYTSVSRPGRGEPRFISVGYVDGDEIFHVDSDAASPREEPRLEEFEQEGPSYGDRNTQIYKAQAQTDRESLRNLRGYYNQSEAGSHTLQSMYGCDVGPDGRLLRGHDQYAYDGKDYIALNEDLRSWTAADTAAQITQRKWETQKEAEQRRAYLEGECVEWLRRYLENGKDKLERADQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGSHHHHHHLE

SEQ ID NO: 687 - protein, 260 aa, synthetic construct
MGSHSMRYFYTSVSRPGRGEPRFISVGYVDGDEIFHVDSDAASPREEPRLEEFEQEGPSYGDRNTQIYKAQAQTDRESLRNLRGYYNQSEAGSHTLQSMYGCDVGPDGRLLRGHDQYAYDGKDYIALNEDLRSWTAADTAAQITQRKWETQKEAEQRRAYLEGECVEWLRRYLENGKDKLERADMKAIFVLNAQHDEAVDANSLAEAKVLANRELDKYGVSDYYKNLINNAKTVEGVKALIDEILAALPGGSHHHHHHLE

SEQ ID NO: 688 - protein, 271 aa, synthetic construct
MGSHSMRYFYTSVSRPGRGEPRFISVGYVDGDEIFHVDSDAASPREEPRLEEFEQEGPSYGDRNTQIYKAQAQTDRESLRNLRGYYNQSEAGSHTLQSMYGCDVGPDGRLLRGHDQYAYDGKDYIALNEDLRSWTAADTAAQITQRKWETQKEAEQRRAYLEGECVEWLRRYLENGKDKLERADMKAIFVLNAQHDEAVDANSLAEAKVLANRELDKYGVSDYYKNLINNAKTVEGVKALIDEILAALPGGSHHHHHHLESEDDEEKEEDG

SEQ ID NO: 689 - protein, 313 aa, synthetic construct
MSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGLVMAPRTVLLLLSAALALTETWAGSHSMRYFYTSVSRPGRGEPRFISVGYVDDDQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADGGSHHHHHH

SEQ ID NO: 690 - protein, 359 aa, synthetic construct
MGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTAGGSHHHHHHSEDDEEKEEDG

SEQ ID NO: 691 - protein, 346 aa, synthetic construct
MIKEEHVIIQAEFYLNPDQSGEFMFDFDGDEIFHVDMAKKETVWRLEEFEQEGPSYGDRNTQIYKAQAQTDRESLRNLRGYYNQSEAGSHTLQSMYGCDVGPDGRLLRGHDQYAYDGKDYIALNEDLRSWTAADTAAQITQRKWETQKEAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTAGGSHHHHHHLE

SEQ ID NO: 692 - protein, 347 aa, synthetic construct
MIKEEHVIIQAEFYLNPDQSGEFMFDFDGDEIFHVDMAKKETVWRLEEFEQEGPSYGDRNTQIYKAQAQTDRESLRNLRGYYNQSEAGSHFLWQLKFECHFFNGTERVRLLERCIYDGKDYIALNEDLRSWTAADTAAQITQRKWETQKEAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTAGGSHHHHHHLE

SEQ ID NO: 693 - protein, 192 aa, synthetic construct
MIKEEHVIIQAEFYLNPDQSGEFMFDFDGDEIFHVDMAKKETVWRLEEFEQEGPSYGDRNTQIYKAQAQTDRESLRNLRGYYNQSEAGSHFLWQLKFECHFFNGTERVRLLERCIYDGKDYIALNEDLRSWTAADTAAQITQRKWETQKEAEQRRAYLEGECVEWLRRYLENGKDKLERADGGSHHHHHHLE

SEQ ID NO: 694 - protein, 290 aa, synthetic construct
MIKEEHVIIQAEFYLNPDQSGEFMFDFDGDEIFHVDMAKKETVWRLEEFEQEGPSYGDRNTQIYKAQAQTDRESLRNLRGYYNQSEAGSHFLWQLKFECHFFNGTERVRLLERCIYDGKDYIALNEDLRSWTAADTAAQITQRKWETQKEAEQRRAYLEGECVEWLRRYLENGKDKLERADQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGSHHHHHHLE

SEQ ID NO: 695 - protein, 397 aa, synthetic construct
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGSGGSGGSGGSGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEGGSHHHHHHLE

SEQ ID NO: 696 - protein, 409 aa, synthetic construct
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMTSGGGGSGGGGSGGGGSGGGGSSSGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEGGSHHHHHHLE

SEQ ID NO: 697 - protein, 405 aa, synthetic construct
GSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWESGGGGSGGGGSGGGGSGGGGIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGSHHHHHHLE

SEQ ID NO: 698 - protein, 415 aa, synthetic construct
GSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWESGGGGSGGGGSGGGGSGGGGSGGGGSGGGGIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGSHHHHHHLE

SEQ ID NO: 699 - protein, 308 aa, synthetic construct
GSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADSGGGGSGGGGSGGGGIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGSHHHHHHLE

SEQ ID NO: 700 - protein, 317 aa, synthetic construct
GSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADTSGGGGSGGGGSGGGGSGGGGSSSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGSHHHHHHLE

SEQ ID NO: 701 - protein, 308 aa, synthetic construct
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMSGGGGSGGGGSGGGGGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADGGSHHHHHHLE

SEQ ID NO: 702 - protein, 317 aa, synthetic construct
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMTSGGGGSGGGGSGGGGSGGGGSSSGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADGGSHHHHHHLE

SEQ ID NO: 703 - protein, 200 aa, synthetic construct
CGGGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADGGCGGSHHHHHHLE

SEQ ID NO: 704 - protein, 194 aa, synthetic construct
GSHSMRYYYTSVSRPGRGEPRFNSTGYVDDTQFVRFDSDAASPREEPRAPWIEQEGPEYWDRNTQIYKAQAQTDRESLRNLRGYYNQSEAGSHTLQSKYGCDVGPDGRLLRGHDQYAYDGKDYIALNEDLRSWTAADTAAQITQRKWEAAREAEQRRAYLEGECVEWLRRYLENGKDKLERADGGSHHHHHHLE

SEQ ID NO: 705 - protein, 194 aa, synthetic construct
GSHSMRYNYTSVSRPGRGEPRFLSEGHVDDTQFVRFDSDAASPREEPRAPWIEQEGPEYWDRNTQIYKAQAQTDRESLRNLRGYYNQSEAGSHTLQSIYGCDVGPDGRLLRGHDQYYYDGKDYISLNEDLRSWTAADTAAQITQRKWEAAREAEQRRAYLEGECVEWLRRYLENGKDKLERADGGSHHHHHHLE

SEQ ID NO: 706 - protein, 194 aa, synthetic construct
GSHSMRYNYTSVSRPGRGEPRFLSEGHVDGQPFVRFDSDAASPREEPRAPWIEQEGPEYWDRNTQIYKAQAQTDRESLRNLRGYYNQSEAGLHSLNSIYVCDVGPDGRLLRGHDHYYYDGELYISLNEDLRSWTAADTAAQITQRKWEAAREAEQRRAYLEGECVEWLRRYLENGKDKLERADGGSHHHHHHLE

SEQ ID NO: 707 - protein, 196 aa, synthetic construct
EPHSLRYNLTVLSWDGSVQSGFLTEVHLDGQPFLRCDRQKCRAKPQGQWAEDVLGNKTWDRNTQIYKAQAQTDRESLRNLRGYYNQKEGLHSLQEIRVCEIHEDNSTRSSQHFYYDGELFLSQNLETKEWTMPQSSRADTAAQITQRKWEAAREAEQRRAYLEGECVEWLRRYLENGKDKLERADGGSHHHHHHLE

SEQ ID NO: 708 - protein, 194 aa, synthetic construct
GSHSMRYNYTSVSRPGRGEPRFISEGYVDDTQFVRFDSDAASPREEPRAPWIEQEGPEYWDRNTQIYKAQAQTDRESLRNLRGYYNQSEAGSHTLQSMYGCDVGPDGRLLRGHDQYYYDGKDYISLNEDLRSWTAADTAAQITQRKWEAAREAEQRRAYLEGECVEWLRRYLENGKDKLERADGGSHHHHHHLE

SEQ ID NO: 709 - protein, 194 aa, synthetic construct
GSHSMRYNYMSLSRPGRGEPRFLSEGHVDGTPFVRFDSDAASPREEPRAPWIEQEGPEYWDRNTQIYKAQAQTDRESLRNLRGYYNQSEAGLHSLQSIYVCEVGPDGRLLRGSDHYYYNGKLYISLNEDLRSWTAADTAAQITQRKWEAAREAEQRRAYLEGECVEWLRRYLENGKDKLERADGGSHHHHHHLE

SEQ ID NO: 710 - protein, 191 aa, synthetic construct
EPHSLRYNLMVLSQDGSVQSGFLAEGHLDGQPFLRYDRQKRRAKPRAPWIEQEGPEYWDRNTQIYKAQAQTDRESLRNLRGYYNQSEAGSHSLQEIRVCEIHEDSSTRGSRHFYYNGELFLSQNLETQESTAADTAAQITQRKWEAAREAEQRRAYLEGECVEWLRRYLENGKDKLERADGGSHHHHHHLE

SEQ ID NO: 711 - protein, 194 aa, synthetic construct
GSHSMRYFYTSVSRPGRGEPRFISVGYVDDTQFVRFDSDAASPREEPRAPWIEQEGPESWDRNTQIYKAQAQTDRESLRNLRGRYNQSEAGSHTLQSMYGCDVGPDGRLLRGHDQYAYDGKDYIALNEDLRSWTAADTAAQPTQRYWEAAREAEQRRAYLEGECVEHLRRYLENGKDKLERADGGSHHHHHHLE

SEQ ID NO: 712 - protein, 194 aa, synthetic construct
GSHSMRYFYTSVSRPGRGEPRFISVGYVDDTQFVRFDSDAASPREEPRAPWIEQSGPESWDRNTQIYKAQAQTDRESLRTLRGRYNQSEAGSHTLQSMYGCDVGPDGRLLRGHDQYAYDGKDFIALNEDLRSWTAADTAAQPTQRYWEAAREAEQRRAYLEGECVEHLRLYLENGKDKLERADGGSHHHHHHLE

SEQ ID NO: 713 - protein, 194 aa, synthetic construct
GSHSMRYFYTSVSRPGRGEPRFISVGYVDDTQFVRFDSDAASPREEPRAPWIEQEGPEGWDRNTQGYKAQAQTDRESLRNMRGYYNQSEAGSHTLQSMYGCDVGPDGRLLRGHDQYAYDGKDYIALNEDLRSWTAADTAAQIDQRKWEAAREAEQRRAYLEGECVEWLRRYLENGKDKLERADGGSHHHHHHLE

SEQ ID NO: 714 - protein, 194 aa, synthetic construct
GSHSMRYFYTSVSRPGRGEPRFISVGYVDDTQFVRFDSDAASPREEPRAPWIEQEGPEGWDRNPQGYKAQAQTDRESLRNMRGRYNQSEAGSHTLQSMYGCDVGPDGRLLRGHDQYAYDGKDYIALNEDLRSWTAADTAAQIDQRYWEAAREAEQRRAYLEGECVEWLRRYLENGKDKLERADGGSHHHHHHLE

SEQ ID NO: 715 - protein, 194 aa, synthetic construct
GSHSMRYFYTSVSRPGRGEPRFISVGYVDDTQFVRFDSDAASPREEPRAPWIEQEGPEAWDRNTQIYKAQAQTDRESLRNLRGYYNQSEAGSHTLQSMYGCDVGPDGRLLRGHDQYAYDGKDSIALNEDLRSWTAADTAAQITQRYWEAAREAEQRRAYLEGECVEHLRRVLENGKDKLERADGGSHHHHHHLE

SEQ ID NO: 716 - protein, 194 aa, synthetic construct
GSHSMRYFYTSVSRPGRGEPRFISVGYVDDTQIVRFDSDAASPREEPRAPFIEQAGPEAWDRNTQIYKAQAQTDRASLRILRGYYNQSEAGSHTLQSMYGCDVGPDGRLLRGHDQYAYDGKDSIALNEDLRSWTAADTAAQITQRYWEAAREAEQRRAYLEGECVEHLRGVLENGKDKLERADGGSHHHHHHLE

SEQ ID NO: 717 - protein, 428 aa, synthetic construct
KPVSKMRMATPLLMQALPMGGGGSGGGGGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRASGGGGSGGGGSGGGGIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSGGSHHHHHHLE

SEQ ID NO: 718 - protein, 400 aa, synthetic construct
GDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRASGGGGSGGGGSGGGGIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSGGSHHHHHHLE

SEQ ID NO: 719 - protein, 409 aa, synthetic construct
GDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRATSGGGGSGGGGSGGGGSGGGGSSSIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSGGSHHHHHHLE

SEQ ID NO: 720 - protein, 400 aa, synthetic construct
IKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSSGGGGSGGGGSGGGGGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRAGGSHHHHHHLE

SEQ ID NO: 721 - protein, 409 aa, synthetic construct
IKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSTSGGGGSGGGGSGGGGSGGGGSSSGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRAGGSHHHHHHLE

SEQ ID NO: 722 - protein, 304 aa, synthetic construct
IKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSSGGGGSGGGGSGGGGGDTRPRFLWQLKFECHFFNGTERVRLLERCIYNQEESVRFDSDVGEYRAVTELGRPDAEYWNSQKDLLEQRRAAVDTYCRHNYGVGESFTVQRRGGSHHHHHHLE

SEQ ID NO: 723 - protein, 329 aa, synthetic construct
IKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSSGGGGSGGGGSGGGGGDTRPRFLWQLKFECHFFNGTERVRLLERCIYNQEESVRFDSDVGEYRAVTELGRPDAEYWNSQKDLLEQRRAAVDTYCRHNYGVGESFTVQRRGGSGGSKPVSKMRMATPLLMQALPMGGSHHHHHHLE

SEQ ID NO: 724 - protein, 299 aa, synthetic construct
GDTRPRFLWQLKFECHFFNGTERVRLLERCIYNQEESVRFDSDVGEYRAVTELGRPDAEYWNSQKDLLEQRRAAVDTYCRHNYGVGESFTVQRRASGGGGSGGGIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSGGSHHHHHHLE

SEQ ID NO: 725 - protein, 328 aa, synthetic construct
KPVSKMRMATPLLMQALPMASGGGGSGGGGDTRPRFLWQLKFECHFFNGTERVRLLERCIYNQEESVRFDSDVGEYRAVTELGRPDAEYWNSQKDLLEQRRAAVDTYCRHNYGVGESFTVQRRASGGGGSGGGIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSGGSHHHHHHLE

SEQ ID NO: 726 - protein, 299 aa, synthetic construct
GDTRPRFLWQLKFECHFFNGTERVRLLERCIYNQEESVRFDSDVGEYRAVTELGRPDAEYWNSQKDLLEQRRAAVDTYCRHNYGVGESFTVQTRSGGGGSGGGGIKEEHVIIQAEFYLNPDQSGEFMFDFDGTEFFHVDMAKKETVWRLEEWGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSGGSHHHHHHLE

SEQ ID NO: 727 - protein, 218 aa, synthetic construct
KPVSKMRMATPLLMQALPMASGGGGSGGGGDTRPRFLWQLKFECHFFNGTERVRLLERCIYNQEESVRFDSDVGEYRAVTELGRPDAEYWNSQKDLLEQRRAAVDTYCRHNYGVGESFTVQRRSGGGIKEEHVIIQAEFYLNPDQSGEFMFDFDGDEIFHVDMAKKETVWRLEEFGRFASFEAQGALANIAVDKANLEIMTKRSNYTGGSHHHHHHLE

SEQ ID NO: 728 - protein, 189 aa, synthetic construct
GDTRPRFLWQLKFECHFFNGTERVRLLERCIYNQEESVRFDSDVGEYRAVTELGRPDAEYWNSQKDLLEQRRAAVDTYCRHNYGVGESFTVQRRSGGGIKEEHVIIQAEFYLNPDQSGEFMFDFDGDEIFHVDMAKKETVWRLEEFGRFASFEAQGALANIAVDKANLEIMTKRSNYTGGSHHHHHHLE

SEQ ID NO: 729 - protein, 210 aa, synthetic construct
IKEEHVIIQAEFYLNPDQSGEFMFDFDGDEIFHVDMAKKETVWRLEEFGRFASFEAQGALANIAVDKANLEIMTKRSNYTGDTRPRFLWQLKFECHFFNGTERVRLLERCIYNQEESVRFDSDVGEYRAVTELGRPDAEYWNSQKDLLEQRRAAVDTYCRHNYGVGESFTVQRRGSGSGSKPVSKMRMATPLLMQALPMGGSHHHHHHLE

SEQ ID NO: 730 - protein, 183 aa, synthetic construct
IKEEHVIIQAEFYLNPDQSGEFMFDFDGDEIFHVDMAKKETVWRLEEFGRFASFEAQGALANIAVDKANLEIMTKRSNQSEAGSHLWQLKFECHFFNGTERVRLLERCIYNQEESVRFDSDVGEYRAVTELGRPDAEYWNSQKDLLEQRRAAVDTYCRHNYGVGESFTVQRRGGSHHHHHHLE

SEQ ID NO: 731 - protein, 183 aa, synthetic construct
IKEEHVIIQAEFYLNPDQSGEFMFDFDGDEIFHVDMAKKETVWRLEEFGRFASFEAQGALANIAVDKANLEIMTKRYNQSEAGSFLWQLKFECHFFNGTERVRLLERCIYNQEESVRFDSDVGEYRAVTELGRPDAEYWNSQKDLLEQRRAAVDTYCRHNYGVGESFTVQRRGGSHHHHHHLE

SEQ ID NO: 732 - protein, 182 aa, synthetic construct
IKEEHVIIQAEFYLNPDQSGEFMFDFDGDEIFHVDMAKKETVWRLEEFGRFASFEAQGALANIAVDKANLEIMTKRSNSGGGGFLWQLKFECHFFNGTERVRLLERCIYNQEESVRFDSDVGEYRAVTELGRPDAEYWNSQKDLLEQRRAAVDTYCRHNYGVGESFTVQRRGGSHHHHHHLE

SEQ ID NO: 733 - protein, 181 aa, synthetic construct
IKEEHVIIQAEFYLNPDQSGEFMFDFDGDEIFHVDMAKKETVWRLEEFGRFASFEAQGALANIAVDKANLEIMTKRSNYTPRFLWQLKFECHFFNGTERVRLLERCIYNQEESVRFDSDVGEYRAVTELGRPDAEYWNSQKDLLEQRRAAVDTYCRHNYGVGESFTVQRRGGSHHHHHHLE

SEQ ID NO: 734 - protein, 185 aa, synthetic construct
IKEEHVIQQSEYYTNPDQSGEFQFDFDGDEIFHVDMAKKETVWRLEEFGRFASFEAQGALANIAVDKANLEIMTKRSNYTGDTRPRFNWQLKFECHFFNGTERVRLLERCNYNQEESVRFDSDVGEYRAVTELGRPDAEYWNSQKDLLEQRRAAVDTYCRHNYGVGESFTVQRRGGSHHHHHHLE

SEQ ID NO: 735 - protein, 183 aa, synthetic construct
IKEEHVIIQAEFYLNPDQSGEFMFDFDGDEIFHVDMAKKETVWRLEEFGRFASFEAQGALANIAVDKANLEIMTKYYNQSEAGSHLWQLKFECHFFNGTERVRLLERYIYNQEEYIRFDSDVGEWRAVTELGRITAEYWNSQKDLLEQRRAAVDTYCRHNYGVGESFTVQRRGGSHHHHHHLE

SEQ ID NO: 736 - protein, 183 aa, synthetic construct
IKEEHVIIQAEFYLNPDQSGEFMFDFDGTEFFHVDMAKKETVWRLEEWGRFASFEAQGALANIAVDKANLEIMTKRSNQSEAGSHLWQLKFECHFFNGTERVRLLERCIYNQEESVRFDSDVGEYRAVTELGRPDAEYWNSQKDLLEQRRAAVDTYCRHNYGVYESWTVQRRGGSHHHHHHLE

SEQ ID NO: 737 - protein, 183 aa, synthetic construct
IKEEHVIRQNETYLNPDQSGLFEFHLDGDPIFRVDMAKKETRWRLEEFGRFASFEAQGALANIAVDKANLEIMTKRSNQSEAGSHSWQLIFVCEFHNGTERVRLSEHCYYNQELSVSFDSDVGEYRAVTELGRPDAEYWNSQKDLLEQRRAAVDTYCRHNYGVGESFTVQRRGGSHHHHHHLE

SEQ ID NO: 738 - protein, 183 aa, synthetic construct
IKEEHVIRQNETYLNPDQSGEFEFDLDGDEIFRVDMAKKETRWRLEEFGRFASFEAQGALANIAVDKANLEIMTKRSNQSEAGSHSWQLKFECEFHNGTERVRLSERCYYNQEESVRFDSDVGEYRAVTELGRPDAEYWNSQKDLLEQRRAAVDTYCRHNYGVGESFTVQRRGGSHHHHHHLE

SEQ ID NO: 739 - protein, 182 aa, synthetic construct
EPHSLRYNLTVLSWDGSVQSGFLTEVHLDGQEIFHVDMAKKETVWRLEEFGRFASFEAQGALANIAVDKANLEIMTKRDQKEGLHSLQEIRVCEIHEDNSTRSSQHFYYDGELFLSQNSDVGEYRAVTELGRPDAEYWNSQKDLLEQRRAAVDTYCRHNYGVGESFTVQRRGGSHHHHHHLE

SEQ ID NO: 740 - protein, 183 aa, synthetic construct
IKEEHSIRQNEMYLNPDQSGLFEFHLDGQPIFRVDMQKKRTKWQLEEFGRFASFEAQGALANIAVDKANLEIMTKRSNQSEAGSHSWQLIFVCEFHEGTESVRLSERCYYNQELSVSFNSDVGEYRAVTELGRPDAEYWNSQKDLLEQRRAAVDTYCRHNYGVGESFTVQRRGGSHHHHHHLE

SEQ ID NO: 741 - protein, 183 aa, synthetic construct
IKEEHSIRQNEFYLNPDQSGEFEFDLDGDEIFRVDMQKKETKWRLEEFGRFASFEAQGALANIAVDKANLEIMTKRSNQSEAGSHSWQLKFECEFHEGTERVRLSERCYYNQEESVRFDSDVGEYRAVTELGRPDAEYWNSQKDLLEQRRAAVDTYCRHNYGVGESFTVQRRGGSHHHHHHLE

SEQ ID NO: 742 - protein, 183 aa, synthetic construct
EPHSLRYNLMVLSQDGSVQSGFLAEGHLDGQPFLRYDMAKKETVWRLEEFGRFASFEAQGALANIAVDKANLEIMTKRSDQKGGLHSLQEIRVCEIHEDSSTRGSRHFYYNGELFLSQNSDVGEYRAVTELGRPDAEYWNSQKDLLEQRRAAVDTYCRHNYGVGESFTVQRRGGSHHHHHHLE

SEQ ID NO: 743 - protein, 185 aa, synthetic construct
IKEEHVIIQAEFYLNPDQSGEFMFDFDGHEIFHVDMAKKETVWRLEEFGRFASFEAQGALANIAVDKANLEIMTKRSNYTGDTRPRFLWQLKFECHFFNGTERVRLLERCIYNQEESVRFDSDVGEYRAVTELGRPDAEYWNSQKDLLEQRRAAVDTYCRHNYGVGESFFVQRRGGSHHHHHHLE

SEQ ID NO: 744 - protein, 305 aa, synthetic construct
GDTRPRFLWQLKFECHFFNGTERVRLLERCIYNQEESVRFDSDVGEYRAVTELGRPDAEYWNSQKDLLEQRRAAVDTYCRHNYGVGESFTVQRRGGYGGWSGGGGSGGGGIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSGGSHHHHHHLE

SEQ ID NO: 745 - protein, 222 aa, synthetic construct
VPTLKFVIFLDVKHFSPEDLTVKGRDIKEEHVIIQAEFYLNPDQSGEFMFDFDGDEIFHVDMAKKETVWRLEEFGRFASFEAQGALANIAVDKANLEIMTKRSNYTPGGSMGDTRPRFLWQLKFECHFFNGTERVRLLERCIYNQEESVRFDSDVGEYRAVTELGRPDAEYWNSQKDLLEQRRAAVDTYCRHNYGVGESFTVQGGSHHHHHHLECCPGCCLE

SEQ ID NO: 746 - protein, 196 aa, synthetic construct
SWDPFRDWIKEEHVIIQAEFYLNPDQSGEFMFDFDGDEIFHVDMAKKETVWRLEEFGRFASFEAQGALANIAVDKANLEIMTKRSNYTPGGSMGDTRPRFLWQLKFECHFFNGTERVRLLERCIYNQEESVRFDSDVGEYRAVTELGRPDAEYWNSQKDLLEQRRAAVDTYCRHNYGVGESFTVQGGSHHHHHHLE

SEQ ID NO: 747 - protein, 293 aa, synthetic construct
SHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPGGSHHHHHHLE

SEQ ID NO: 748 - protein, 164 aa, synthetic construct
PENYLFQGRQECNGTQFLERYIYNREEFVRFDEFRAVGRPDEEYWNSQKDILEEERAVPDRMCRHNYELGGPMTLQRRVQPRVNIKADHVSTYAAFVQRPFMFEFDEDEMFYVDLHLEEFGQAFSFEAQGGLANIAILNNNLNTRSNHTQATNDPPHHHHHHLE

SEQ ID NO: 749 - protein, 269 aa, synthetic construct
EDIVADHVASYGVNLYQSYGPSGQYTHEFDGDEQFYVDLGRKETVWCLPVLRQFRFDPQFALTNIAVLKHNLNSLIKRSNSTAATNEVPRDSPEDFVYQFKGMCYFTNGTERVRLVSRSIYNREEIVRFDSDVGEFRAVTLLGLPAAEYWNSQKDILERKRAAVDRVCRHNYQLELRTTLQRRVEPGGSMKAIFVLNAQHDEAVDANSALAKAKVLANRELDKYGVSDYYKKLVNNAKTVEGVKDLQAQVVESAKKARGGSHHHHHHLE

SEQ ID NO: 750 - protein, 202 aa, synthetic construct
HVLRYGYTGIFDDTSHMTLTVVGIFDGQHFFTYHVQSSDKASSRANGTISWMANVSAAYPTYLDGERAKGDLIFNQTEQNLLELEIALGYRSQSVLTWTHECNTTENGSFVAGYEGFGWDGETLMELKDNLTLWTGPNYEISWLKQQKTYIDGKIKNISEGDTTIQRNYLKGNCTQWSVIYSGFQPPVTHPGGSHHHHHHLE

SEQ ID NO: 751 - protein, 185 aa, synthetic construct
IKEEHVIIQAEFYLNPDQSGEFMFDFDGDEIFHVDMAKKETVWRLEEFGRFASFEAQGALANIAVDKANLEIMTKRSNYTPMGDTRPRFLWQLKFESHFFNGTERVRLLERCIYNQEESVRFDSDVGEYRAVTELGRPDAEYWNSQKDLLEQRRAAVDTYSRHNYGVGESFTVQGGSHHHHHHLE

SEQ ID NO: 752 - protein, 185 aa, synthetic construct
IKEEHVIIQAEFYLNPDQSGEFMFDFDGDEIFHVDMAKKETVWRLEEFGRFASFEAQGALANIAVDKANLEIMTKRSNYTPMGDTRPRFLWQLKFEKHFFNGTERVRLLERCIYNQEESVRFDSDVGEYRAVTELGRPDAEYWNSQKDLLEQRRAAVDTYDRHNYGVGESFTVQGGSHHHHHHLE

SEQ ID NO: 753 - protein, 184 aa, synthetic construct
DTRPRFLWQLKFEKHFFNGTERVRLLERCIYNQEESVRFDSDVGEYRAVTELGRPDAEYWNSQKDLLEQRRAAVDTYDRHNYGVGESFTVQMIKEEHVIIQAEFYLNPDQSGEFMFDFDGDEIFHVDMAKKETVWRLEEFGRFASFEAQGALANIAVDKANLEIMTKRSNYTPGGSHHHHHHLE

SEQ ID NO: 754 - protein, 184 aa, synthetic construct
DTRPRFLWQLKFESHFFNGTERVRLLERCIYNQEESVRFDSDVGEYRAVTELGRPDAEYWNSQKDLLEQRRAAVDTYSRHNYGVGESFTVQMIKEEHVIIQAEFYLNPDQSGEFMFDFDGDEIFHVDMAKKETVWRLEEFGRFASFEAQGALANIAVDKANLEIMTKRSNYTPGGSHHHHHHLE

SEQ ID NO: 755 - protein, 188 aa, synthetic construct
HVLRYGYTGIFDDTSHMTLTVVGIFDGQHFFTYHVQSSDKASSRANGLEEFGRFASFEAQGALANIAVDKANLEIMTKRSGYRSQSVLTWTHEKNTTENGSFVAGYEGFGWDGETLMELKDNLTLWTGPNYEIAVTELGRPDAEYWNSQKDLLEQRRAAVDTYDRHNYGVGESFTVQGGSHHHHHHLE

SEQ ID NO: 756 - protein, 188 aa, synthetic construct
HVLRYGYTGIFDDTSHMTLTVVGIFDGQHFFTYHVQSSDKASSRANGAVTELGRPDAEYWNSQKDLLEQRRAAVDTYCRHNYGVGESGYRSQSVLTWTHECNTTENGSFVAGYEGFGWDGETLMELKDNLTLWTGPNYEILEEFGRFASFEAQGALANIAVDKANLEIMTKRSFTVQGGSHHHHHHLE

SEQ ID NO: 757 - protein, 193 aa, synthetic construct
GGGSGGGGSDTRPRFLWQLKFEKHFFNGTERVRLLERCIYNQEESVRFDSDVGEYRAVTELGRPDAEYWNSQKDLLEQRRAAVDTYDRHNYGVGESFTVQMIKEEHVIIQAEFYLNPDQSGEFMFDFDGDEIFHVDMAKKETVWRLEEFGRFASFEAQGALANIAVDKANLEIMTKRSNYTPGGSHHHHHHLE

SEQ ID NO: 758 - protein, 164 aa, synthetic construct
PENYLFQGRQEKNGTQFLERYIYNREEFVRFDEFRAVGRPDEEYWNSQKDILEEERAVPDRMDRHNYELGGPMTLQRRVQPRVNIKADHVSTYAAFVQRPFMFEFDEDEMFYVDLHLEEFGQAFSFEAQGGLANIAILNNNLNTRSNHTQATNDPPHHHHHHLE

SEQ ID NO: 759 - protein, 195 aa, synthetic construct
SHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGKDVGPDGRLLRGHDQYAYDGKDYIALNEDLRSWTAADTAAQITQRKWEAAREAEQRRAYLEGEDVEWLRRYLENGKDKLERADPPGGSHHHHHHLE

SEQ ID NO: 760 - protein, 408 aa, synthetic construct
GSSHHHHHHGGGGSGGGGSGDTRPRFLWQLKFEKHFFNGTERVRLLERCIYNQEESVRFDSDVGEYRAVTELGRPDAEYWNSQKDLLEQRRAAVDTYDRHNYGVGESFTVQRRVEPKVTVYPSKTQPLQHHNLLVDSVSGFYPGSIEVRWFRNGQEEKAGVVSTGLIQNGDWTFQTLVMLETVPRSGEVYTKQVEHPSVTSPLTVEWRASGGGGSGGGGSGGGGIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLIDFIDKFTPPVVNVTWLRNGKPVTTGVSETVFLPREDHLFRKFHYLPFLPSTEDVYDKRVEHWGLDEPLLKHWEFDAPS

SEQ ID NO: 761 - protein, 287 aa, synthetic construct
GKEEHVIIQAEFYLNPDQSGEFMFDFDGDEIFHVDMAKKETVWRLEEFEQEGPSYGDRNTQIYKAQAQTDRESLRNLRGYYNQSEAGSHFLWQLKFEKHFFNGTERVRLLERCIYDGKDYIALNEDLRSWTAADTAAQITQRKWETQKEAEQRRAYLEGEDVEWLRRYLENGKDKLERADQRTPKIQVYSRHPAENGKSNFLNKYVSGFHPSDIEVDLLKNGERIEKVEHSDLSFSKDWSFYLLYYTEFTPTEKDEYADRVNHVTLSQPKIVKWDRDMGGSHHHHHH

SEQ ID NO: 762 - protein, 283 aa, synthetic construct
GKEEHVIIQAEFYLNPDQSGEFMFDFDGDEIFHVDMAKKETVWRLEEFGRFASFEAQGALANIAVDKANLEIMTKRSNYTPMGDTRPRFLWQLKFEKHFFNGTERVRLLERCIYNQEESVRFDSDVGEYRAVTELGRPDAEYWNSQKDLLEQRRAAVDTYDRHNYGVGESFTVQQRTPKIQVYSRHPAENGKSNFLNKYVSGFHPSDIEVDLLKNGERIEKVEHSDLSFSKDWSFYLLYYTEFTPTEKDEYADRVNHVTLSQPKIVKWDRDMGGSHHHHHHLE

SEQ ID NO: 763 - protein, 292 aa, synthetic construct
GSHSMRYYYTSVSRPGRGEPRFNSTGYVDDTQFVRFDSDAASPREEPRAPWIEQEGPEYWDRNTQIYKAQAQTDRESLRNLRGYYNQSEAGSHTLQSKYGCDVGPDGRLLRGHDQYAYDGKDYIALNEDLRSWTAADTAAQITQRKWEAAREAEQRRAYLEGECVEWLRRYLENGKDKLERADQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGSHHHHHHLE

SEQ ID NO: 764 - protein, 294 aa, synthetic construct
GSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGSHHHHHHLE

SEQ ID NO: 765 - protein, 292 aa, synthetic construct
GSHSMRYFYTSVSRPGRGEPRFISVGYVDDTQFVRFDSDAASPREEPRAPWIEQEGPEAWDRNTQIYKAQAQTDRESLRNLRGYYNQSEAGSHTLQSMYGCDVGPDGRLLRGHDQYAYDGKDSIALNEDLRSWTAADTAAQITQRYWEAAREAEQRRAYLEGECVEHLRRVLENGKDKLERADQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGSHHHHHHLE

SEQ ID NO: 766 - protein, 398 aa, synthetic construct
GDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRASGGGGSGGGGSGGGGIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSGGSHHHHHH

SEQ ID NO: 767 - protein, 398 aa, synthetic construct; identical to SEQ ID NO: 766

SEQ ID NO: 768 - protein, 404 aa, synthetic construct
MVKYQKGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRASGGGGSGGGGSGGGGIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSGGSHHHHHH

SEQ ID NO: 769 - protein, 403 aa, synthetic construct
MRRRIGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRASGGGGSGGGGSGGGGIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSGGSHHHHHH

SEQ ID NO: 770 - protein, 403 aa, synthetic construct
MRIRRGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRASGGGGSGGGGSGGGGIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSGGSHHHHHH

SEQ ID NO: 771 - protein, 403 aa, synthetic construct
MRRIRGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRASGGGGSGGGGSGGGGIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSGGSHHHHHH

SEQ ID NO: 772 - protein, 405 aa, synthetic construct
MRIRRGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRASGGGGSGGGGSGGGGIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSGGSHHHHHHLE

SEQ ID NO: 773 - protein, 403 aa, synthetic construct
MIRPRGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRASGGGGSGGGGSGGGGIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSGGSHHHHHH

SEQ ID NO: 774 - protein, 403 aa, synthetic construct
GSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWESGGGGSGGGGSGGGGSGGGGIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGSHHHHHH

SEQ ID NO: 775 - protein, 403 aa, synthetic construct; identical to SEQ ID NO: 774

SEQ ID NO: 776 - protein, 409 aa, synthetic construct
MVKYQKGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWESGGGGSGGGGSGGGGSGGGGIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGSHHHHHH

SEQ ID NO: 777 - protein, 408 aa, synthetic construct
MRRRIGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWESGGGGSGGGGSGGGGSGGGGIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGSHHHHHH

SEQ ID NO: 778 - protein, 408 aa, synthetic construct
MRIRRGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWESGGGGSGGGGSGGGGSGGGGIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGSHHHHHH

SEQ ID NO: 779 - protein, 408 aa, synthetic construct
MRRIRGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWESGGGGSGGGGSGGGGSGGGGIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGSHHHHHH

SEQ ID NO: 780 - protein, 410 aa, synthetic construct
MRIRRGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWESGGGGSGGGGSGGGGSGGGGIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGSHHHHHHLE

SEQ ID NO: 781 - protein, 408 aa, synthetic construct
MIRPRGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWESGGGGSGGGGSGGGGSGGGGIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGSHHHHHH

SEQ ID NO: 782 - protein, 180 aa, synthetic construct
MGDTRPRFLWQPKRECHFFNGTERVRFLDRYFYNQEESVRFDSDVGEFRAVTELGRPDAEYWNSQKDILEQARAAVDTYCRHNYGVVESFTVQRRVIKEEHVIIQAEFYLNPDQSGEFMFDFDGDEIFHVDMAKKETVWRLEEFGRFASFEAQGALANIAVDKANLEIMTKRSNYTPITN

SEQ ID NO: 783 - protein, 189 aa, synthetic construct
MGDTRPRFLWQPKRECHFFNGTERVRFLDRYFYNQEESVRFDSDVGEFRAVTELGRPDAEYWNSQKDILEQARAAVDTYCRHNYGVVESFTVQRRVIKEEHVIIQAEFYLNPDQSGEFMFDFDGDEIFHVDMAKKETVWRLEEFGRFASFEAQGALANIAVDKANLEIMTKRSNYTPITNGGSHHHHHH

SEQ ID NO: 784 - protein, 200 aa, synthetic construct
MVKYHHHHHHGGSENLYFQGMGDTRPRFLWQPKRECHFFNGTERVRFLDRYFYNQEESVRFDSDVGEFRAVTELGRPDAEYWNSQKDILEQARAAVDTYCRHNYGVVESFTVQRRVIKEEHVIIQAEFYLNPDQSGEFMFDFDGDEIFHVDMAKKETVWRLEEFGRFASFEAQGALANIAVDKANLEIMTKRSNYTPITN

SEQ ID NO: 785 - protein, 178 aa, synthetic construct
MGDTPENYLFQGRQECYAFNGTQRFLERYIYNREEFVRFDSDVGEFRAVTELGRPDEEYWNSQKDILEEERAVPDRMCRHNYELGGPMTLQRRVIKADHVSTYAAFVQTHRPTGEFMFEFDEDEMFYVDLDKKETVWHLEEFGQAFSFEAQGGLANIAILNNNLNTLIQRSNHTQATN

SEQ ID NO: 786 - protein, 191 aa, synthetic construct
MVKYHHHHHHGGSMGDTPENYLFQGRQECYAFNGTQRFLERYIYNREEFVRFDSDVGEFRAVTELGRPDEEYWNSQKDILEEERAVPDRMCRHNYELGGPMTLQRRVIKADHVSTYAAFVQTHRPTGEFMFEFDEDEMFYVDLDKKETVWHLEEFGQAFSFEAQGGLANIAILNNNLNTLIQRSNHTQATN

SEQ ID NO: 787 - protein, 178 aa, synthetic construct
MGDTPENYLFQGRQECYAFNGTQRFLERYIYNREEFARFDSDVGEFRAVTELGRPAAEYWNSQKDILEEKRAVPDRMCRHNYELGGPMTLQRRVIKADHVSTYAAFVQTHRPTGEFMFEFDEDEMFYVDLDKKETVWHLEEFGQAFSFEAQGGLANIAILNNNLNTLIQRSNHTQATN

SEQ ID NO: 788 - protein, 190 aa, synthetic construct
MVKYHHHHHHGGSGDTPENYLFQGRQECYAFNGTQRFLERYIYNREEFARFDSDVGEFRAVTELGRPAAEYWNSQKDILEEKRAVPDRMCRHNYELGGPMTLQRRVIKADHVSTYAAFVQTHRPTGEFMFEFDEDEMFYVDLDKKETVWHLEEFGQAFSFEAQGGLANIAILNNNLNTLIQRSNHTQATN

SEQ ID NO: 789 - protein, 180 aa, synthetic construct
MIKEEHVIIQAEFYLNPDQSGEFMFDFDGDEIFHVDMAKKETVWRLEEFGRFASFEAQGALANIAVDKANLEIMTKRSNYTPTINGDTRPRFLWQLKFECHFFNGTERVRLLERCIYNQEESVRFDSDVGEYRAVTELGRPDAEYWNSQKDLLEQRRAAVDTYCRHNYGVGESFTVQRRV

SEQ ID NO: 790 - protein, 192 aa, synthetic construct
MVKYHHHHHHGGSIKEEHVIIQAEFYLNPDQSGEFMFDFDGDEIFHVDMAKKETVWRLEEFGRFASFEAQGALANIAVDKANLEIMTKRSNYTPTINGDTRPRFLWQLKFECHFFNGTERVRLLERCIYNQEESVRFDSDVGEYRAVTELGRPDAEYWNSQKDLLEQRRAAVDTYCRHNYGVGESFTVQRRV

SEQ ID NO: 791 - protein, 180 aa, synthetic construct
MGDTRPRFLWQLKFECHFFNGTERVRLLERCIYNQEESVRFDSDVGEYRAVTELGRPDAEYWNSQKDLLEQRRAAVDTYCRHNYGVGESFTVQRRVIKEEHVIIQAEFYLNPDQSGEFMFDFDGDEIFHVDMAKKETVWRLEEFGRFASFEAQGALANIAVDKANLEIMTKRSNYTPTIN

SEQ ID NO: 792 - protein, 192 aa, synthetic construct
MVKYHHHHHHGGSGDTRPRFLWQLKFECHFFNGTERVRLLERCIYNQEESVRFDSDVGEYRAVTELGRPDAEYWNSQKDLLEQRRAAVDTYCRHNYGVGESFTVQRRVIKEEHVIIQAEFYLNPDQSGEFMFDFDGDEIFHVDMAKKETVWRLEEFGRFASFEAQGALANIAVDKANLEIMTKRSNYTPTIN

SEQ ID NO: 793 - protein, 189 aa, synthetic construct
MGDTRPRFLWQLKFECHFFNGTERVRLLERCIYNQEESVRFDSDVGEYRAVTELGRPDAEYWNSQKDLLEQRRAAVDTYCRHNYGVGESFTVQRRVIKEEHVIIQAEFYLNPDQSGEFMFDFDGDEIFHVDMAKKETVWRLEEFGRFASFEAQGALANIAVDKANLEIMTKRSNYTPTINGGSHHHHHH

SEQ ID NO: 794 - protein, 183 aa, synthetic construct
MRDSPEDFVYQFKGMCYFTNGTERVRLVSRSIYNREEIVRFDSDVGEFRAVTLLGLPAAEYWNSQKDILERKRAAVDRVCRHNYQLELRTTLQRRVVEDIVADHVASYGVNLYQSYGPSGQYTHEFDGDEQFYVDLGRKETVWCLPVLRQFRFDPQFALTNIAVLKHNLNSLIKRSNSTAATN

SEQ ID NO: 795 - protein, 195 aa, synthetic construct
MVKYHHHHHHGGSRDSPEDFVYQFKGMCYFTNGTERVRLVSRSIYNREEIVRFDSDVGEFRAVTLLGLPAAEYWNSQKDILERKRAAVDRVCRHNYQLELRTTLQRRVVEDIVADHVASYGVNLYQSYGPSGQYTHEFDGDEQFYVDLGRKETVWCLPVLRQFRFDPQFALTNIAVLKHNLNSLIKRSNSTAATN

SEQ ID NO: 796 - protein, 182 aa, synthetic construct
MGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEDIVADHVASYGVNLYQSYGPSGQYTHEFDGDEQFYVDLGRKETVWCLPVLRQFRFDPQFALTNIAVLKHNLNSLIKRSNSTAATN

SEQ ID NO: 797 - protein, 194 aa, synthetic construct
MVKYHHHHHHGGSGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEDIVADHVASYGVNLYQSYGPSGQYTHEFDGDEQFYVDLGRKETVWCLPVLRQFRFDPQFALTNIAVLKHNLNSLIKRSNSTAATN

SEQ ID NO: 798 - protein, 179 aa, synthetic construct
GIKEEHVIIQAEFYLNPDQSGEFMFDFDGDEIFHVDMAKKETVWRLEEFGRFASFEAQGALANIAVDKANLEIMTKRSNYTPITNGDTQPRFLEQAKCECHFLNGTERVWNLIRYIYNQEEYARYNSDLGEYQAVTELGRPDAEYWNSQKDLLERRRAEVDTYCRYNYGVVESFTVQRR

SEQ ID NO: 799 - protein, 178 aa, synthetic construct
GDTQPRFLEQAKCECHFLNGTERVWNLIRYIYNQEEYARYNSDLGEYQAVTELGRPDAEYWNSQKDLLERRRAEVDTYCRYNYGVVESFTVQRRIKEEHVIIQAEFYLNPDQSGEFMFDFDGDEIFHVDMAKKETVWRLEEFGRFASFEAQGALANIAVDKANLEIMTKRSNYTPITN

SEQ ID NO: 800 - protein, 181 aa, synthetic construct
GRDPPEDFVLQFKAMCYFTNGTERVRYVTRYIYNREEDVRFDSDVGVYRAVTPQGRPDAEYWNSQKDILERTRAELDTVCRHNYEVAFRGILQRREDIVADHVASYGVNLYQSYGPSGQFTHEFDGDEEFYVDLERKETVWKLPLFHRLRFDPQFALTNIAVLKHNLNILIKRSNSTAATN

SEQ ID NO: 801 - protein, 181 aa, synthetic construct
GEDIVADHVASYGVNLYQSYGPSGQFTHEFDGDEEFYVDLERKETVWKLPLFHRLRFDPQFALTNIAVLKHNLNILIKRSNSTAATNRDPPEDFVLQFKAMCYFTNGTERVRYVTRYIYNREEDVRFDSDVGVYRAVTPQGRPDAEYWNSQKDILERTRAELDTVCRHNYEVAFRGILQRR

SEQ ID NO: 802 - protein, 181 aa, synthetic construct
GEDIVADHVASYGVNLYQSYGPSGQYTHEFDGDEQFYVDLGRKETVWCLPVLRQFRFDPQFALTNIAVLKHNLNSLIKRSNSTAATNRDSPEDFVYQFKGMCYFTNGTERVRLVSRSIYNREEIVRFDSDVGEFRAVTLLGLPAAEYWNSQKDILERKRAAVDRVCRHNYQLELRTTLQRR

SEQ ID NO: 803 - protein, 177 aa, synthetic construct
GDTRPRFLWQLIFVCEFHEGTESVRLSERCYYNQELSVSFNSDVGEYRAVTELGRPDAEYWNSQKDLLEQRRAAVDTYCRHNYGVGESFTVQRRSGGGKEEHSIRQNEMYLNPDQSGLFEFHLDGQPIFRVDMQKKRTKWQLEEFGRFASFEAQGALANIAVDKANLEIMTKRSNYT

SEQ ID NO: 804 - protein, 173 aa, synthetic construct
GIKEEHSIRQNEMYLNPDQSGLFEFHLDGQPIFRVDMQKKRTKWQLEEFGRFASFEAQGALANIAVDKANLEIMTKRSNQSEAGSHSWQLIFVCEFHEGTESVRLSERCYYNQELSVSFNSDVGEYRAVTELGRPDAEYWNSQKDLLEQRRAAVDTYCRHNYGVGESFTVQRR

SEQ ID NO: 805 - protein, 181 aa, synthetic construct
GSHSMRYYYTSVSRPGRGEPRFNSTGYVDDTQFVRFDSDAASPRYEPRAPWIEQETPEYWDRNTQDYKAQAQTDREYLRNLRWYYNQSEAGSHTLQSKYGCDVGPDGRLLRGHDQYAYDGKDYIALNEDLRSWTAADTAAQYTQRLPEAARLAEQRRATLEGLCVEWLRRSLENGKDKLER

SEQ ID NO: 806 - protein, 181 aa, synthetic construct
GSHSMRYYDTSVSRPGRGEPRFNSTGYVDDTQFVRFDSDAASPREEPRAPWIEQEGPEYWDRNTQDYKAQAQDDRMYLRNLRGWYNQSEAGSHTLQSKDGCDVGPDGRLLRGHDQLAYDGKDYIALNEDLRSWTAADTAAQILQRLWEAARLAELIRAYLEGERVEWLRRDLENGKDKLER

SEQ ID NO: 807 - protein, 181 aa, synthetic construct
GSHSMRYYDTSVSRPGRGEPRFNSTGYVDDTQFVRFDSDAASPRMEPRAPWIEQEGPEPVDRNTQIYKAQAQTDRESLRNLRGYYNQSEAGSHTLQSKAGCDVGPDGRLLRGHDQWAYDGKDYIALNEDLRSWTAADTAAQITQRHWEAAREAEQRRAYLEGECVETLRRQLENGKDKLER

SEQ ID NO: 808 - protein, 181 aa, synthetic construct; identical to SEQ ID NO: 807

SEQ ID NO: 809 - protein, 181 aa, synthetic construct; identical to SEQ ID NO: 807

SEQ ID NO: 810 - protein, 183 aa, synthetic construct
GSHSMRYFYTSVSRPGRGEPRFISVGYVDDTQFVRFDSDAASPREEPRAPWIEQEGPEGWDRNTQGYKAQAQTDRESLRNMRGYYNQSEAGSHTLQSMYGCDVGPDGRLLRGHDQYAYDGKDYIALNEDLRSWTAADTAAQIDQRKWEAAREAEQRRAYLEGECVEWLRRYLENGKDKLERAD

SEQ ID NO: 811 - protein, 173 aa, synthetic construct
GIKEEHSIRQNEKYLNPDQSGNFEFHLDGQPIFRVDMQKKRTKWQLEEFGRFASFEAQGALANIAVDKANLEIMTKRSNQSEAGSHSWQLNFVCEFHEGTESVRLSERCYYNQENSVSFNSDVGEYRAVTELGRPDAEYWNSQKDLLEQRRAAVDTYCRHNYGVGESFTVQRR

SEQ ID NO: 812 - protein, 173 aa, synthetic construct
GIKEEHSIRQNEKYNNPDQSGNFEFHLDGQPIFRVDMQKKRTKWQLEEFGRFASFEAQGALANIAVDKANLEIMTKRSNQSEAGSHSWQLNFVCEFHEGTESVRLSERCYYNQENSVSFNSDVGEYRAVTELGRPDAEYWNSQKDLLEQRRAAVDTYCRHNYGVGESFTVQRR

SEQ ID NO: 813 - protein, 173 aa, synthetic construct
GIKEEHSIRQNEKYNNPDQSGNFEFHLDGQPIFRVDMQKKRTKWQLEEFGRFASFEAQGALANIAVDKANLEIMTKRSNQSEAGSHSWQLNFTCEFHEGTESVRLSERCYYNQENSVSFNSDVGEYRATTELGRPDAEYWNSQKDLLEQRRAAVDTYCRHNYGVGESFTVQRR

SEQ ID NO: 814 - protein, 173 aa, synthetic construct
GIKEEHSIRQNEKYNNPDQSGNFEFHLDGQPIFRVDMQKKRTKWQLEEFGRFASFEAQGALANIAVFKINLEIMTKRSNQSEAGSHSWQLNFTCEFHEGTESVRLSERSYYNQENFVSFNSDVGEYRATTELGRPMAEYVNSQKDTLEQRRAAVDTYCRHNYGVGEYFTVQRR

SEQ ID NO: 815 - protein, 173 aa, synthetic construct
GIKEEHSIRYNEKYNNPDQSGNFEFHLDGQPIWRVDMQKKRTKWQLEEFGRFASFEAQQALANIADDKYNLEIMTSRSNQSEAGSHSWQLNFTCEFHEGTESVRLSNRCYYNQENSVSFNSDVGEYRATTELGRPDAEYWNSQKDSLEQRRAIVDTYCRHNYGVGESFTVQRR

SEQ ID NO: 816 - protein, 173 aa, synthetic construct
GIKEEHSIRQNEKYNNPDQSGNFEFHLDGQPISRVDMQKKRTKWQLEEFGRYASFEAQEALAFIAVDKANLEIMTKRSNQSEAGSHSWQLNFTCEFHEGTESVRLSERCYYNQENSVSFNSDVGEYRATTELGRPIAEYGNSQKDLLEKRRAIVDTSCRHNYGVGEEFTVQRR

SEQ ID NO: 817 - protein, 173 aa, synthetic construct
GIKEEHSIRQNVKYNNPDQSGNFEFHLDGQPIFRVDMQKKRTKWQLEEFGREPSFEAQGYLANIAVDKYNLEIMTKNSNQSEAGSHSWQLNMTCEFHEGTESVRLSERDYYNQENSVSFNSDVGEYRATTELGRPDAEYRNSQKDLLEQRRAVVDTVCRHNYGVGESFTVNRR

SEQ ID NO: 818 - protein, 173 aa, synthetic construct
GIKEEHSYRTNEKYNNPDQSGNFEFHLDGQPIFRVDMQKKRTKWQLEEFGREASFEAQPYLANIAVDKANLEIMTKRSNQSEAGSHSWQINFTCEFHEGTESVRLSERCYYNQENSVSFNSDVGEYRATTELGRPDANYQNSQKDLLEQRRAWVDTYCRHNYGVGELFTVQRR

SEQ ID NO: 819 - protein, 179 aa, synthetic construct
GDTRPRFLWQLKFECHFYNGTERVRLNERCNFYNQEESVRFDSDVGEYRAVTELGRPDAEYWNSQKDLLEQRRAAVDTYCRHNYGVGESFTVQRRSGGGIKEEHVINQAEYYLNPDQSGEFKFDFDGDEIFHVDMAKKETVWRLEEFGRFASFEAQGALANIAVDKANLEIMTKRSNYT

SEQ ID NO: 820 - protein, 178 aa, synthetic construct
GDTRPRFLWQLKFECHFYNGTERVRLNERCNYNQEESVRFDSDVGEYRAVTELGRPVAEYVNSQKDLLEQHRAAVDTECRHNYGVGESFTVQRRSGGGIKEEHVINQAEYYLNPDQSGEFKFDFDGDEIFHVDMAKKETVWRLEEFGRFASTEAQGALYNIAVSKANLEIMTKRSNYT

SEQ ID NO: 821 - protein, 178 aa, synthetic construct
GDTRPRFLWQLKWECHFYNGTERVRLNERCNYNQEESVRFDSDVGEYRAVTELGRPDAEFSNSQKDNLEQRRAAVDTLCRHNYGVGESFTVQRRSGGGIKEEHVINQAEYYLNPDQSGEFKFDFDGDEIFHVDMAKKETVWRLEEFGRFAIIEAYGALANIAVDKANLEIMTKQSNYT

SEQ ID NO: 822 - protein, 178 aa, synthetic construct
GDTRPRFLWQLKFECHFYNGTERVRLNERCNYNQEESVRFDSDVGEYRAVTELGRPDAEDSNSQKDLLETRRAGVDTYCRHNYGVGESFTVQRRSGGGIKEEHVINQAEYYLNPDQSGEFKFDFDGDEWFHVDMAKKETVWRLEEFGRFASNEAQGALANIAVDVANLEIMLKRSNYT

SEQ ID NO: 823 - protein, 178 aa, synthetic construct
GDTRPRFLWQLKFECHFYNGTERVRLNERNNYNQEESVRFDSDVGEYRAVTELGRPDAEYTNSQKDALEQRRAWVDTYCRMNEGVGESFTVQRRSGGGIKEEHVINQAEYYLNPDQSGEFKTDFDGDEIFHVDMAKKETVWRLEEFGRFASFEAQGALENIAVDKANLEIMTKRSNYT

SEQ ID NO: 824 - protein, 178 aa, synthetic construct
GDTRPRFLWQLKFECHFYNGTERVRLNERSNYNQEESVRFDSDVGEYRAVTELGRPDAEYWNSGKDQLEQRRAAVDTICRFNYGVGESFTVQRRSGGGIKEEHVINVAEYYLNPDQSGEFKFDFDGDEIFHVDMAKKETVWRLEEFGRFNSFEAEGALANIAVDKANLEIMTKRSNYT

SEQ ID NO: 825 - protein, 184 aa, synthetic construct
MIKEEHSIRQNEKYLNPDQSGNFEFHLDGQPIFRVDMQKKRTKWQLEEFGRFASFPAQGALANIAVDKANLEIMTKRSNQSEAGSHSWQLNFVCEFHEGTESVRLSERCYYNQELSVSFNSDVGEYRAVTELGRPDAEYWNSQKDLLEQRRAAVDHVCRHNYGVGESFTVQRRGGSHHHHHHLE

SEQ ID NO: 826 - protein, 184 aa, synthetic construct
MIKEEHSIRWNEKYLNPDQSGNFEFHLDGQPIFRVDMQKKRTKWQLEEFGRFASFEAQFPLAIIAVKKANLEKMTKHSNQSEAGSHSWQHNFVCEFHEGTESVRLSERCYYNQELSVSFNSDVGEGRAVTELGRPDAEYHNSQKDLLEQRRAAVDHYCRYNYGVGESFTVQRRGGSHHHHHHLE

SEQ ID NO: 827 - protein, 186 aa, synthetic construct
MIKEEHVINQAEYYLNPDQSGEFKFDFDGDEIFHVDMAKKETVWRLEEFGRFASFEAQGALAHIAVDKANLEIMTKRSNYTPMGDTRPRFLWQLKYECHFFNGTERVRLNERCIYNQEESVRFDSDVGEYRAVTELGRPDAEQWNSQKDHLEQRRAAVDTKCRHNYGVGESFTVQGGSHHHHHHLE

SEQ ID NO: 828 - protein, 186 aa, synthetic construct
MIKEEHVINQAEYYLNPDQSGEFKFDFDGDEIFHVDMAKKETVWRLEEFGRFASMEAQGWLARIAVDKANLEIMTVPSNYTPMGDTRPRFLWQLKFECHFFNGTERVRLNERDIYNQEESVRFDSDVGENRAVTELGRPTAEDKNSQKDLLEQRRAAVDTYCRDNYGVGESFTVQGGSHHHHHHLE

SEQ ID NO: 829 - protein, 186 aa, synthetic construct
MIKEEHVINYAEYYLNPDQSGEFKFDFDGDEIFHVDMAKKETVWRLEEFGRFASFEAQGPLANIAVDKANLESMTKDSNYTPMGDTRPRFLYQLKFECHFFNGTERVRLNERCIYNQEESVRFDSDVGEYRAVTELGRKDAEHHNSQKDLLEQRRAAVDKACRHAYGSGESFTVQGGSHHHHHHLE

SEQ ID NO: 830 - protein, 189 aa, synthetic construct
MKEEHVIIQAEFYLNPDQSGEFMFDFDGDEIFHVDMAKKETVWRLEEFGRFASFEAQGALANIAVDKANLEIMTKRSNYTPITNTRPRFLWQPKRECHFFNGTERVRFLDRYFYNQEESVRFDSDVGEFRAVTELGRPDAEYWNSQKDILEQARAAVDTYCRHNYGVVESFTVQRRVQGGSHHHHHHLE

SEQ ID NO: 831 - protein, 194 aa, synthetic construct
MSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEGGSHHHHHHLE

SEQ ID NO: 832 - protein, 194 aa, synthetic construct
MSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADGGSHHHHHHLE

SEQ ID NO: 833 - protein, 191 aa, synthetic construct
MGSHSLRYFSTAVSRPGRGEPRYIAVEYVDDTQFLRFDSDAAIPRMEPREPWVEQEGPQYWEWTTGYAKANAQTDRVALRNLLRRYNQSEAGSHTLQGMNGCDMGPDGRLLRGYHQHAYDGKDYISLNEDLRSWTAADTVAQITQRFYEAEEYAEEFRTYLEGECLELLRRYLENGKETLGGSHHHHHHLE

SEQ ID NO: 834 - protein, 189 aa, synthetic construct
MGSHLSLLYHITAVTDPHGGTPSFFVSGWLGPQQYLSYSNRRSEAEPYGAWIWESQVSWYWEKETVDLKNKQQLFLEALEVLGEGSYTLQGLLGCELGPDNASVPTAKFALNGEDFMSFDPKQGSWAGEWPEALRISTRWQQQAEAVSKERTFLLTSCPQRLLGHLERGRGNLEWKEPGGSHHHHHHLE

SEQ ID NO: 835 - protein, 186 aa, synthetic construct
EPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVGGSHHHHHH

SEQ ID NO: 836 - protein, 187 aa, synthetic construct
MGRSHSLHYLFMGASEQDLGLSLFEALGYVDDQLFVFYDHESRRVEPRTPWVSSRISSQMWLQLSQSLKGWDHMFTVDFWTIMENHNHSKESHTLQVILGCEMQEDNSTEGYWKYGYDGQDHLEFCPDTLDWRAAEPRAWPTKLEWERHKIRARQNRAYLERDCPAQLQQLLELGRGVGGSHHHHHH

SEQ ID NO: 837 - protein, 184 aa, synthetic construct
MGRTHSLRYFRLGVSDPIHGVPEFISVGYVDSHPITTYDSVTRQKEPRAPWMAENLAPDHWERYTQLLRGWQQMFKVELKRLQRHYNHSGSHTYQRMIGCELLEDGSTTGFLQYAYDGQDFLIFNKDTLSWLAVDNVAHTIKQAWEANQHELLYQKNWLEEECIAWLKRFLEYGKGGSHHHHHH

SEQ ID NO: 838 - protein, 186 aa, synthetic construct
MGSDGLQRLHMLQISYFRDPYHVWYQGNASLGGHLTHVLEGPDTNTTIIQLQPLQEPESWARTQSGLQSYLLQFHGLVRLVHQERTLAFPLTIRCFLGCELPPEGSRAHVFFEVAVNGSSFVSFRPERALWQADTQVTSGVVTFTLQQLNAYNRTRYELREFLEDTCVQYVQKHISAGGSHHHHHH

SEQ ID NO: 839 - protein, 185 aa, synthetic construct
MGIKEEHVINQAEYCLNPDQSGEFKFDFDGDEIFHVDMAKKETVWRLEEFGRFASFEAQGALAHIAVDCANLEIMTKRSNYTPMGDTRPRFLWQLKYECHFFNGTERVRLNERCIYNQEESVRFDSDVGEYRAVTELGRPDAEQWNSQKDHLEQRRAAVDTKCRHNYGVGESFTVQGGSHHHHHH

SEQ ID NO: 840 - protein, 186 aa, synthetic construct
MGKEEHVIIQAEFCLNPDQSGEFMFDFDGDEIFHVDMAKKETVWRLEEFGRFASFEAQGALANIAVDCANLEIMTKRSNYTPITNTRPRFLWQPKRECHFFNGTERVRFLDRYFYNQEESVRFDSDVGEFRAVTELGRPDAEYWNSQKDILEQARAAVDTYCRHNYGVVESFTVQRRGGSHHHHHH

SEQ ID NO: 841 - protein, 209 aa, synthetic construct
MGSKARMATGALAQAGGGGSGGGGSMIKEEHVINQAEYYLNPDQSGEFKFDFDGDEIFHVDMAKKETVWRLEEFGRFASFEAQGALAHIAVDKANLEIMTKRSNYTPMGDTRPRFLWQLKYECHFFNGTERVRLNERCIYNQEESVRFDSDVGEYRAVTELGRPDAEQWNSQKDHLEQRRAAVDTKCRHNYGVGESFTVQGGSHHHHHH

SEQ ID NO: 842 - protein, 216 aa, synthetic construct
MGKPVSKMRMATPLLMQALPMASGGGGSGGGGMIKEEHVINQAEYYLNPDQSGEFKFDFDGDEIFHVDMAKKETVWRLEEFGRFASFEAQGALAHIAVDKANLEIMTKRSNYTPMGDTRPRFLWQLKYECHFFNGTERVRLNERCIYNQEESVRFDSDVGEYRAVTELGRPDAEQWNSQKDHLEQRRAAVDTKCRHNYGVGESFTVQGGSHHHHHH

SEQ ID NO: 843 - protein, 205 aa, synthetic construct
MGSKARMATGALAQAGGSGGSMIKEEHVINQAEYYLNPDQSGEFKFDFDGDEIFHVDMAKKETVWRLEEFGRFASFEAQGALAHIAVDKANLEIMTKRSNYTPMGDTRPRFLWQLKYECHFFNGTERVRLNERCIYNQEESVRFDSDVGEYRAVTELGRPDAEQWNSQKDHLEQRRAAVDTKCRHNYGVGESFTVQGGSHHHHHH

SEQ ID NO: 844 - protein, 207 aa, synthetic construct
MGIKEEHVINQAEYYLNPDQSGEFKFDFDGDEIFHVDMAKKETVWRLEEFGRFASFEAQGALAHIAVDKANLEIMTKRSNYTPMGGGSKARMATGALAQAGGSGGSGDTRPRFLWQLKYECHFFNGTERVRLNERCIYNQEESVRFDSDVGEYRAVTELGRPDAEQWNSQKDHLEQRRAAVDTKCRHNYGVGESFTVQGGSHHHHHH

SEQ ID NO: 845 - protein, 186 aa, synthetic construct
MGKEEHVINQAEYYLNPDQSGEFKFDFDGDEIFHVDMAKKETVWRLEEFGRFASFEAQGALANIAVDKANLEIMTKRSNYTPITNTRPRFLWQPKRECHFFNGTERVRFNDRYFYNQEESVRFDSDVGEFRAVTESGRPDAEYDNSQKDTLEVARAMVDTNCRHRYGHVESFTVQRRGGSHHHHHH

SEQ ID NO: 846 - protein, 186 aa, synthetic construct
MGKEEHVINQAEYYLNPDQSGEFKFDFDGDEIFHVDMAKKETVWRLEEFGRFASFEAQGALANIAVDKANLEIMTKRSNYTPITNTRPRFLWQPKRECHFFNGTERVRFNDRYFYNQEESVRFDSDVGEFRAVTEPGRPEAEYRNSQKDMLEAARAQVDTMCRHTYGQVESFTVQRRGGSHHHHHH

SEQ ID NO: 847 - protein, 198 aa, synthetic construct
MAPTPMWPDDLQNHTFLHTVYCQDGSPSVGLSEAYDEDQLFFFDFSQNTRVPRLPEFADWAQEQGDAPAILFDKEFCEWMIQQIGPKLDGKIPGGFVAHVESTCLLDDAGTPKDFTYCISFNKDLLTCWDPEENKMAPCEFGVLNSLANVLSQHLNQKDTLMQRLRNGLQNCATHTQPFWGSLTNRTRPGGSHHHHHH

SEQ ID NO: 848 - protein, 186 aa, synthetic construct
MVKEEHVINQAEYYLNPDQSGEFKFDFDGDEIFHVDMAKKETVWRLEEFGRFASFEAQGALANIAVDKANLEIMTKRSNYTPMGDTRPRFLWQLKFECHFFNGTERVRLNERCIYNQEESVRFDSDVGEYRAVTELGRPDAEYWNSQKDLLEQRRAAVDTYCRHNYGVGESFTVQGGSHHHHHHLE

SEQ ID NO: 849 - protein, 185 aa, synthetic construct
MGIKEEHSIRQNEKYLNPDQSGNFEFHLDGQPIFRVDMQKKRTKWQLEEFGRFASFEAQGALANIAVDKANLEIMTKRSNQSEAGSHSWQLNFVCEFHEGTESVRLSERCYYNQENSVSFNSDVGEYRAVTELGRPDAEYWNSQKDLLEQRRAAVDTYCRHNYGVGESFTVQRRGGSHHHHHHLE

SEQ ID NO: 850 - protein, 186 aa, synthetic construct; identical to SEQ ID NO: 848

SEQ ID NO: 851 - protein, 185 aa, synthetic construct; identical to SEQ ID NO: 849

SEQ ID NO: 852 - protein, 200 aa, synthetic construct
MGSHLSLLYHITAVTDPHGGTPSFFVSGWLGPQQYLSYSNRRSEAEPYGAWIWESQVSWYWEKETVDLKNKQQLFLEALEVLGEGSYTLQGLLGCELGPDNASVPTAKFALNGEDFMSFDPKQGSWAGEWPEALRISTRWQQQAEAVSKERTFLLTSCPQRLLGHLERGRGNLEWKEPVSGWRLFKKISGKQKGSGSGEK

SEQ ID NO: 853 - protein, 200 aa, synthetic construct
MGRSHSLHYLFMGASEQDLGLSLFEALGYVDDQLFVFYDHESRRVEPRTPWVSSRISSQMWLQLSQSLKGWDHMFTVDFWTIMENHNHSKESHTLQVILGCEMQEDNSTEGYWKYGYDGQDHLEFCPDTLDWRAAEPRAWPTKLEWERHKIRARQNRAYLERDCPAQLQQLLELGRGVVSGWRLFKKISGKQKGSGSGEK

SEQ ID NO: 854 - protein, 186 aa, synthetic construct
IKEEHVIIQAEFYLNPDQSGEFQFDFDGDEIFHVDMAKKETVWRLEEFGRFASFEAQEALANIAVDKAKLEIMTKRSNYTPMGDTRPRFLSQSKFECHFFNGTERVRLNERCIYNQEESVRFDSDVGEYRAVTELGRPDAEYWNSQKDLLEQRRAAVDEYCRHNYGVGESFTVQRGGSHHHHHHLE

SEQ ID NO: 855 - protein, 186 aa, synthetic construct
IKEEHVIIQAEFYLNPDQSGEFQGDFDGDEIFHVDMAKKETVWRLEEFGRQASFEAQGALANIAVDKANLEIMTKRSNYTPMGDTRPRFLWQLKFECHFFNGTERVRLNERCIYNQEESVRFDSDVGEYRAVTELGRPDAEKWNSQKDLLEQRRAAVDLHCRHNYGVGESFTVQRGGSHHHHHHLE

SEQ ID NO: 856 - protein, 189 aa, synthetic construct
IKEEHVIIQAEFYLNPDQSGEFKFDFDGDEIFHVDMAKKETVWRLEEFGRFASFEAQRALANIAVDKANLEIMTKRSNYTPMGDTRPRFLWQLKFECHFFNGTERVRLLERCIYNQEESVRFDSDVGEYRAVTELGRPDAEYWNSQKDLLEQRRAAVDTQCRHNYGVGESFTVQRRVEGGSHHHHHHLE

SEQ ID NO: 857 - protein, 189 aa, synthetic construct
IKEEHVIIQAEFYLNPDQSGEFKFDFDGDEIFHVDMAKKETVWRLEEFGRFASFEAQGALANIAVDKANLEKMTKRSNYTPMGDTRPRFLWQLKFECHFFNGTERVRLLERCIYNQEESVRFDSDVGEYRAVTELGRPDAEYSNSQKDLLEQRRAAVDTQCRHNYGVGESFTVQRRVEGGSHHHHHHLE

SEQ ID NO: 858 - protein, 193 aa, synthetic construct
SHSMRYNYTSVSRPGRGEPRFNSEGYVDDTQFVRFDSDAASPREEPRAPWIEQEGPEDWDRNTQIYKAQAQTDRESLRNLRGYYNQSEAGSHTLQSQYGCDVGPDGRLLRGHDQYYYDGKDYISLNEDLRSWTAADTAAQITQRKAEAARIAEQRRAYLEGECVEWLRRYLENGKDKLERADGGSHHHHHHLE

SEQ ID NO: 859 - protein, 193 aa, synthetic construct
SHSMRANYTSVSRPGRGEPRFNSEGYVDDTQFVRFDSDAASPREEPRAPWIEQEGPEYWDRNTQIYKAQAQTDRESLRNLRGYYNQSEAGSHTLQSQVGCDVGPDGRLLRGHDQVYYDGKDYISLNEDLRSWTAADTAAQITQRKNEAAREAEQRRAYLEGECVEWLRRYLENGKDKLERADGGSHHHHHHLE

SEQ ID NO: 860 - protein, 189 aa, synthetic construct
DTRPRFLWQLKFECHFFNGTERVRLLERCIYNQEESVRFDSDVGEYRAVTELGRPDAEKWNSQKDLLEQRRAAVDTYCRHNYGVGESFTVQRRSGGGIKEEHVIIQAEYYLNPDQSGEFQVDFDGDEIFHVDMAKKETVWRLEEFGRFASFEAQLALANIAVDKANLEIMTKRSNYTPGGSHHHHHHLE

SEQ ID NO: 861 - protein, 189 aa, synthetic construct
DTRPRFLWQLKFECHFFNGTERVRLLERCIYNQEESVRFDSDVGEYRAVTELGRPDAEYVNSQKDQLEQRRAAVDTYCRHNYGVGESFTVQRRSGGGIKEEHVIIQAEYYLNPDQSGEFQFDFDGDEIFHVDMAKKETVWRLEEFGRFASFEAQGALANIAQDKHNLEIMTKRSNYTPGGSHHHHHHLE

SEQ ID NO: 862 - protein, 183 aa, synthetic construct
IKEEHSIRQNEQYLNPDQSGNFEFHLDGQPIFRVDMQKKRTKWQLEEFGRFASFEAQEALAGIAVDKANLEIMTKRSNQSEAGSHSWQLNFVCEFHEGTESVRLSERCYYNQELSVSFNSDVGEYRAVTELGRPDAEYKNSQKDLLEQRRAAVDTYCRHNYGVGESFTVQRRGGSHHHHHHLE

SEQ ID NO: 863 - protein, 183 aa, synthetic construct
IKEEHSIRQNEQYLNPDQSGNFEFHLDGQPIFRVDMQKKRTKWQLEEFGRFASEEAQGALANIAHDKANLEIMTKRSNQSEAGSHSWQLNFVCEFHEGTESVRLSERCYYNQELSVSFNSDVGEYRAVTELGRPDAEKANSQKDNLEQRRAAVDTYCRHNYGVGESFTVQRRGGSHHHHHHLE

SEQ ID NO: 864 - protein, 190 aa, synthetic construct
PENYLFQGRQECYAFNGTQRFLERANYNREEFVRFDSDVGEFRAVTELGRPDEEYWNSQKDILEEERAVPDRACRHNYELGGPMTLQRRVQPRVNIKADHVSTYAAYVQTHRPTGEFKFEFDEDEMFYVDLDKKETVWHLEEFGQAFSFEAQGGLANIAILNNNLNTLIQRSNHTQATNDPPHHHHHHLE

SEQ ID NO: 865 - protein, 190 aa, synthetic construct
PENYLFQGRQECYAFNGTQRFLERYNYNREEIVRFDSDVGEFRAVTELGRPDEEYWNSQKDILEEERAVPDRMCRHNYELGGPMTLQRRVQPRVNIKADHVSTYAAYVQTHRPTGEFKFEFDEDEMFYVDLDKKETVWHLEEFGQAFSFEAQEGLALIAILNNNLNKLIQRSNHTQATNDPPHHHHHHLE

SEQ ID NO: 866 - protein, 186 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTERGRPTAEYYNSQKDVLESARATVDTYCRHGYGRVESFTVQRRVQGGSHHHHHH

SEQ ID NO: 867 - protein, 177 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTEGGRPQAEYQNSQKDVLEAARAGVDTTCRHRYGRVESFTVQRRVQ

SEQ ID NO: 868 - protein, 177 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTEKGRPQAEYRNSQKDLLEHARADVDTPCRHRYGEVESFTVQRRVQ

SEQ ID NO: 869 - protein, 177 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTEGGRPMAEYANSQKDLLEHARAVVDTACRHSYGVVESFTVQRRVQ

SEQ ID NO: 870 - protein, 177 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTEGGRPPAEYRNSQKDSLEKARASVDTLCRHPYGLVESFTVQRRVQ

SEQ ID NO: 871 - protein, 177 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTEFGRPLAEYSNSQKDALERARAMVDTSCRHRYGPVESFTVQRRVQ

SEQ ID NO: 872 - protein, 177 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTEMGRPVAEYANSQKDFLESARAKVDTTCRHRYGRVESFTVQRRVQ

SEQ ID NO: 873 - protein, 177 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTEPGRPIAEYRNSQKDSLELARARVDTSCRHRYGMVESFTVQRRVQ

SEQ ID NO: 874 - protein, 177 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTETGRPAAEYTNSQKDLLESARAIVDTVCRHLYGLVESFTVQRRVQ

SEQ ID NO: 875 - protein, 177 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTEDGRPYAEYHNSQKDYLEIARAYVDTMCRHFYGSVESFTVQRRVQ

SEQ ID NO: 876 - protein, 177 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTEKGRPAAEYLNSQKDLLEWARAFVDTDCRHFYGSVESFTVQRRVQ

SEQ ID NO: 877 - protein, 186 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTEFGRPYAEYGNSQKDLLEVARAFVDTVCRHQYGQVESFTVQRRVQGGSHHHHHH

SEQ ID NO: 878 - protein, 177 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTERGRPAAEYRNSQKDKLEPARAVVDTMCRHPYGLVESFTVQRRVQ

SEQ ID NO: 879 - protein, 177 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTEPGRPMAEYLNSQKDQLEVARAIVDTRCRHTYGTVESFTVQRRVQ

SEQ ID NO: 880 - protein, 177 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTEGGRPHAEYVNSQKDELEWARAQVDTTCRHLYGMVESFTVQRRVQ

SEQ ID NO: 881 - protein, 177 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTEVGRPIAEYWNSQKDTLELARAAVDTLCRHGYGLVESFTVQRRVQ

SEQ ID NO: 882 - protein, 186 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTEYGRPGAEYGNSQKDKLEEARALVDTQCRHLYGVVESFTVQRRVQGGSHHHHHH

SEQ ID NO: 883 - protein, 177 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTERGRPDAEYLNSQKDRLETARADVDTPCRHGYGSVESFTVQRRVQ

SEQ ID NO: 884 - protein, 177 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTETGRPDAEYGNSQKDALEDARAMVDTLCRHSYGVVESFTVQRRVQ

SEQ ID NO: 885 - protein, 177 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTEVGRPDAEYLNSQKDQLEAARAVVDTMCRHVYGWVESFTVQRRVQ

SEQ ID NO: 886 - protein, 186 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTEDGRPDAEYANSQKDALESARASVDTSCRHRYGYVESFTVQRRVQGGSHHHHHH

SEQ ID NO: 887 - protein, 177 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTEGGRPDAEYVNSQKDRLEEARARVDTLCRHGYGRVESFTVQRRVQ

SEQ ID NO: 888 - protein, 177 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTEFGRPDAEYVNSQKDFLEHARARVDTFCRHGYGHVESFTVQRRVQ

SEQ ID NO: 889 - protein, 177 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTEYGRPDAEYLNSQKDRLELARAGVDTGCRHYYGLVESFTVQRRVQ

SEQ ID NO: 890 - protein, 186 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTEFGRPDAEYLNSQKDWLEKARAFVDTACRHRYGAVESFTVQRRVQGGSHHHHHH

SEQ ID NO: 891 - protein, 177 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTEIGRPDAEYANSQKDILEAARALVDTGCRHGYGMVESFTVQRRVQ

SEQ ID NO: 892 - protein, 186 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTESGRPTAEYLNSQKDILEQARAAVDTHCRHPYGAVESFTVQRRVQGGSHHHHHH

SEQ ID NO: 893 - protein, 177 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTELGRPLAEYVNSQKDLLERARATVDTDCRHNYGVVESFTVQRRVQ

SEQ ID NO: 894 - protein, 177 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTEYGRPFAEYWNSQKDVLEQARAAVDTLCRHVYGLVESFTVQRRVQ

SEQ ID NO: 895 - protein, 177 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTELGRPSAEYYNSQKDPLEHARAAVDTICRHSYGVVESFTVQRRVQ

SEQ ID NO: 896 - protein, 177 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTELGRPFAEYKNSQKDFLEQARAIVDTQCRHWYGVVESFTVQRRVQ

SEQ ID NO: 897 - protein, 177 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTELGRPDAEYVNSQKDVLEWARAHVDTPCRHVYGVVESFTVQRRVQ

SEQ ID NO: 898 - protein, 177 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTELGRPDAEYRNSQKDFLEEARAAVDTVCRHGYGGVESFTVQRRVQ

SEQ ID NO: 899 - protein, 186 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTERGRPSAEYWNSQKDTLELARAAVDTGCRHMYGVVESFTVQRRVQGGSHHHHHH

SEQ ID NO: 900 - protein, 177 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTELGRPGAEYPNSQKDYLEWARAAVDTTCRHVYGVVESFTVQRRVQ

SEQ ID NO: 901 - protein, 177 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTELGRPNAEYWNSQKDHLEGARAAVDTRCRHQYGRVESFTVQRRVQ

SEQ ID NO: 902 - protein, 177 aa, synthetic construct
KEEHVIIQAEFYLNPDQSGEFMFDFDGDEIFHVDMAKKETVWRLEEFGRFASFEAQGALANIAVDKANLEIMTKRSNYTPITNTRPRFLWQPKRECHFFNGTERVRFLDRYFYNQEESVRFDSDVGEFRAVTELGRPDAEYWNSQKDILEQARAAVDTYCRHNYGVVESFTVQRRVQ

SEQ ID NO: 903 - protein, 177 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTELGRPDAEYWNSQKDILEQARAAVDTYCRHNYGVVESFTVQRRVQ

SEQ ID NO: 904 - protein, 177 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTEFGRPNAEYMNSQKDLLEVARAMVDTSCRHWYGQVESFTVQRRVQ

SEQ ID NO: 905 - protein, 177 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTEAGRPVAEYRNSQKDMLESARARVDTECRHYYGGVESFTVQRRVQ

SEQ ID NO: 906 - protein, 177 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTEYGRPAAEYLNSQKDWLEMARATVDTVCRHQYGIVESFTVQRRVQ

SEQ ID NO: 907 - protein, 177 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTELGRPFAEYSNSQKDALEPARAFVDTYCRHTYGRVESFTVQRRVQ

SEQ ID NO: 908 - protein, 186 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTEHGRPRAEYENSQKDTLEQARADVDTGCRHDYGPVESFTVQRRVQGGSHHHHHH

SEQ ID NO: 909 - protein, 177 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTERGRPSAEYFNSQKDHLEGARAHVDTICRHGYGAVESFTVQRRVQ

SEQ ID NO: 910 - protein, 177 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTEVGRPGAEYPNSQKDELELARAAVDTKCRHAYGLVESFTVQRRVQ

SEQ ID NO: 911 - protein, 177 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTESGRPEAEYWNSQKDSLEKARASVDTRCRHLYGMVESFTVQRRVQ

SEQ ID NO: 912 - protein, 177 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTEGGRPQAEYQNSQKDVLEAARAGVDTTCRHRYGHVESFTVQRRVQ

SEQ ID NO: 913 - protein, 177 aa, synthetic construct
KEEHVIIQAEYYLNPDQSGEFKFDFDGDEIFHVDMAKKETVWRLEEFGRFASFEAQGALANIAVDKANLEIMTKRSNYTPITNTRPRFLWQPKRECHFYNGTERVRFLDRYYYNQEESVRFDSDVGEFRAVTEKGRPWAEYANSQKDKLENARALVDTLCRHMYGNVESFTVQRRVQ

SEQ ID NO: 914 - protein, 246 aa, synthetic construct
  VARIANT 84: note = Any amino acid
  VARIANT 120: note = Any amino acid
  VARIANT 224: note = Any amino acid
IKRSKKNSLALSLTADQMVSALLDAEPPILYSEYDPTRPFSEASMMGLLTNLADRELVHMINWAKRVPGFVDLTLHDQVHLLEXAWLEILMIGLVWRSMEHPGKLLFAPNLLLDRNQGKXVEGMVEIFDMLLATSSRFRMMNLQGEEFVCLKSIILLNSGVYTFEKDHIHRVLDKITDTLIHLMAKAGLTLQQQHQRLAQLLLILSHIRHMSNKGMEHLYSMKXKNVVPLSDLLLEMLDAHRLHAP

SEQ ID NO: 915 - protein, 245 aa, synthetic construct
PYASLTEIEHLVQSVCKSYRETCQLRLEDLLRQRSNIFSREEVTGYQRKSMWEMWERCAHHLTEAIQYVVEFAKRLSGFMELCQNDQIVLLKAGAMEVVLVRMCRAYNADNRTVFFEGKYGGMELFRALGCSELISSIFDFSHSLSALHFSEDEIALYTALVLINAHRPGLQEKRKVEQLQYNLELAFHHHLCKTHRQSILAKLPPKGKLRSLCSQHVERLQIFQHLHPIVVQAAFPPLYKELFS

SEQ ID NO: 916 - protein, 115 aa, synthetic construct
SMPSQMEHAMETMMFTFHKFAGDKGYLTKEDLRVLMEKEFPGFLENQKDPLAVDKIMKDLDQCRDGKVGFQSFFSLIAGLTIACNDYFVVHMKQENLYFQGDSTVHEILSKLSLE

SEQ ID NO: 917 - protein, 189 aa, synthetic construct; identical to SEQ ID NO: 56

SEQ ID NO: 918 - protein, 178 aa, synthetic construct
EATLGSGNLRQAVMLPEGEDLNEWIAVNTVDFFNQINMLYGTITEFCTEASCPVMSAGPRYEYHWADPIKCSAPKYIDYLMTWVQDQLDDETLFPSKIGVPFPKNFMSVAKTILKRLFRVYAHIYHQHFDSVMQLQEEAHLNTSFKHFIFFVQEFNLIDRRELAPLQELIEKLGSKDR

SEQ ID NO: 919 - protein, 142 aa, synthetic construct; identical to SEQ ID NO: 203

SEQ ID NO: 920 - protein, 127 aa, synthetic construct
EDLIDGIIFAANYLGSTQLLSDKTPSKNVRQAQEAVSRIKAQKLTEVDLFILTQRIKVLNADTQETDHPLRTISYIADIGNIVVLARRRYKICHVFESEDAQLIAQSIGQAFSVAYQEFLRANGINP

SEQ ID NO: 921 - protein, 196 aa, synthetic construct
EARLEEAVNRWVLKFYFHEALRAFRGSRYGDFRQIRDIMQALLVRPLGKEHTVSRLLRVMQCLSRIEEGENLDCSFDMEELTPLESAINVLEMIKTEFTLTEAVVESSRKLVKEAAVIICIKNKEFEKASKILKKHMPTTQKLRNDLLNIIREKNLAHPVIQNFSYETFQQKMLRFLESHLDDAEPYLLTMAKKAL

SEQ ID NO: 922 - protein, 38 aa, synthetic construct
ITNATTAATTAATATTTASTEGSNSESEAESTENSPTP

SEQ ID NO: 923 - protein, 30 aa, synthetic construct
HKPLRHPGMPKGLAHQLASPGLTTLPEASD

SEQ ID NO: 924 - protein, 29 aa, synthetic construct
DMQPGHHETSTPAGPSQADLHTPHTEDGG

SEQ ID NO: 925 - protein, 11 aa, synthetic construct
ALVNYIFFKGK

SEQ ID NO: 926 - protein, 42 aa, synthetic construct
MERKFMSLQPSISVSEMEPNGTFSNNNSRNCTIENFKREFFP

SEQ ID NO: 927 - protein, 21 aa, synthetic construct
MERKFMSLQPSISVSEMEPNG

SEQ ID NO: 928 - protein, 21 aa, synthetic construct
SISVSEMEPNGTFSNNNSRNC

SEQ ID NO: 929 - protein, 21 aa, synthetic construct
TFSNNNSRNCTIENFKREFFP

SEQ ID NO: 930 - protein, 43 aa, synthetic construct
GMERKFMSLQPSISVSEMEPNGTFSNNNSRNCTIENFKREFFP

SEQ ID NO: 931 - protein, 21 aa, synthetic construct
QQQEQQPASGGGGQNGQQAMS

SEQ ID NO: 932 - protein, 24 aa, synthetic construct
LPPELRRQRSGEVENNSDNSGRYQ

SEQ ID NO: 933 - protein, 25 aa, synthetic construct
PPKPVSKMRMATPLLMQALPMGALP

SEQ ID NO: 934 - protein, 23 aa, synthetic construct
PGAAAAHLPRWPPPQLAASRREA

SEQ ID NO: 935 - protein, 25 aa, synthetic construct
NKEETKKENSFEMQKGDQNPQIAAH

SEQ ID NO: 936 - protein, 31 aa, synthetic construct
MANFTPVNGSSGNQSVRLVTSSSHNRYETVE

SEQ ID NO: 937 - protein, 33 aa, synthetic construct
MDPTTPAWGTESTTVNGNDQALLLLCGKETLIP

SEQ ID NO: 938 - protein, 35 aa, synthetic construct
MGSPWNGSDGPEGAREPPWPALPPCDERRCSPFPL

SEQ ID NO: 939 - protein, 37 aa, synthetic construct
MDSFNYTTPDYGHYDDKDTLDLNTPVDKTSNTLRVPD

SEQ ID NO: 940 - protein, 38 aa, synthetic construct
MEPLFPASTPSWNASSPGAASGGGDNRTLVGPAPSAGA

SEQ ID NO: 941 - protein, 39 aa, synthetic construct
MERAPPDGPLNASGALAGEAAAAGGARGFSAAWTAVLAA

SEQ ID NO: 942 - protein, 40 aa, synthetic construct
MASSWPPLELQSSNQSQLFPQNATACDNAPEAWDLLHRVL

SEQ ID NO: 943 - protein, 41 aa, synthetic construct
MAQRQPHSPNQTLISITNDTESSSSVVSNDNTNKGWSGDNS

SEQ ID NO: 944 - protein, 46 aa, synthetic construct
MEPSATPGAQMGVPPGSREPSPVPPDYEDEFLRYLWRDYLYPKQYE

SEQ ID NO: 945 - protein, 48 aa, synthetic construct
MFRQEQPLAEGSFAPMGSLQPDAGNASWNGTEAPGGGARATPYSLQVT

SEQ ID NO: 946 - protein, 49 aa, synthetic construct
MEEPGAQCAPPPPAGSETWVPQANLSSAPSQNCSAKDYIYQDSISLPWK

SEQ ID NO: 947 - protein, 52 aa, synthetic construct
MGPIGAEADENQTVEEMKVEQYGPQTTPRGELVPDPEPELIDSTKLIEVQVW

SEQ ID NO: 948 - protein, 52 aa, synthetic construct
MPANFTEGSFDSSGTGQTLDSSPVACTETVTFTEVVEGKEWGSFYYSFKTEQ

SEQ ID NO: 949 - protein, 52 aa, synthetic construct
MRLSAGPDAGPSGNSSPWWPLATGAGNTSREAEALGEGNGPPRDVRNEELAK

SEQ ID NO: 950 - protein, 53 aa, synthetic construct
MKETRGYGGDAPFCTRLNHSYTGMWAPERSAEARGNLTRPPGSGEDCGSVSVA

SEQ ID NO: 951 - protein, 54 aa, synthetic construct
MSGTKLEDSPPCRNWSSASELNETQEPFLNPTDYDDEEFLRYLWREYLHPKEYE

SEQ ID NO: 952 - protein, 58 aa, synthetic construct
MELLKLNRSVQGTGPGPGASLCRPGAPLLNSSSVGNLSCEPPRIRGAGTRELELAIRW

SEQ ID NO: 953 - protein, 68 aa, synthetic construct
MRLNSSAPGTPGTPAADPFQRAQAGLEEALLAPGFGNASGNASERVLAAPSSELDVNTDIYSKVLVTW

SEQ ID NO: 954 - protein, 46 aa, synthetic construct
MAAISTSIPVISQPQFTAMNEPQCFYNESIAFFYNRSGKHLATEWN

SEQ ID NO: 955 - protein, 25 aa, synthetic construct
MILNSSTEDGIKRIQDDCPKAGRHN

SEQ ID NO: 956 - protein, 31 aa, synthetic construct
MDPTISTLDTELTPINGTEETLCYKQTLSLT

SEQ ID NO: 957 - protein, 19 aa, synthetic construct
MSTPGVNSSASLSPDRLNS

SEQ ID NO: 958 - protein, 25 aa, synthetic construct
MDVLSPGQGNNTTSPPAPFETGGNT

SEQ ID NO: 959 - protein, 64 aa, synthetic construct
MDILCEENTSLSSTTNSLMQLNDDTRLYSNDFNSGEANTSDAFNWTVDSENRTNLSCEGCLSPS

SEQ ID NO: 960 - protein, 40 aa, synthetic construct
MASPALAAALAVAAAAGPNASGAGERGSGGVANASGASWG

SEQ ID NO: 961 - protein, 53 aa, synthetic construct
MGAGVLVLGASEPGNLSSAAPLPDGAATAARLLVPASPPASLLPPASESPEPL

SEQ ID NO: 962 - protein, 34 aa, synthetic construct
MGQPGNGSAFLLAPNGSHAPDHDVTQERDEVWVV

SEQ ID NO: 963 - protein, 21 aa, synthetic construct
MSQQNTSGDCLFDGVNELMKT

SEQ ID NO: 964 - protein, 32 aa, synthetic construct
MASLSQLSGHLNYTCGAENSTGASQARPHAYY

SEQ ID NO: 965 - protein, 116 aa, synthetic construct
MKSILDGLADTTFRTITTDLLYVGSNDIQYEDIKGDMASKLGYFPQKFPLTSFRGSPFQEKMTAGDNPQLVPADQVNITEFYNKSLSSFKENEENIQCGENFMDIECFMVLNPSQQ

SEQ ID NO: 966 - protein, 29 aa, synthetic construct
MQGNGSALPNASQPVLRGDGARPSWLASA

SEQ ID NO: 967 - protein, 24 aa, synthetic construct
MGNASNDSQSEDCETRQWLPPGES

SEQ ID NO: 968 - protein, 28 aa, synthetic construct
MQAVDNLTSAPGNTSLCTRDYKITQVLF

SEQ ID NO: 969 - protein, 35 aa, synthetic construct
METPNTTEDYDTTTEFDYGDATPCQKVNERAFGAQ

SEQ ID NO: 970 - protein, 28 aa, synthetic construct
MDETGNLTVSSATCHDTIDDFRNQVYST

SEQ ID NO: 971 - protein, 32 aa, synthetic construct
MDPTVPVFGTKLTPINGREETPCYNQTLSFTV

SEQ ID NO: 972 - protein, 54 aa, synthetic construct
MALTPESPSSFPGLAATGSSVPEPPGGPNATLNSSWASPTEPSSLEDLVATGTI

SEQ ID NO: 973 - protein, 25 aa, synthetic construct
  VARIANT 1..25: note = This sequence may encompass 1-5 GGGGS repeating units
GGGGSGGGGSGGGGSGGGGSGGGGS

SEQ ID NO: 974 - protein, 6 aa, synthetic construct
GGGGGG

SEQ ID NO: 975 - protein, 8 aa, synthetic construct
GGGGGGGG

SEQ ID NO: 976 - protein, 7 aa, synthetic construct
QSEAGSH

SEQ ID NO: 977-978 - skipped (2 entries)

SEQ ID NO: 979 - protein, 6 aa, synthetic construct
HHHHHH

SEQ ID NO: 980 - protein, 24 aa, synthetic construct
  VARIANT 1..24: note = This sequence may encompass 1-3 DYKDDDDK repeating units
DYKDDDDKDYKDDDDKDYKDDDDK

SEQ ID NO: 981 - protein, 8 aa, synthetic construct
WSHPQFEK

SEQ ID NO: 982 - protein, 7 aa, synthetic construct
ENLYFQS

SEQ ID NO: 983 - protein, 65 aa, synthetic construct
MKAIFVLNAQHDEAVDANSLAEAKVLANRELDKYGVSDYYKNLINNAKTVEGVKALIDEILAALP

SEQ ID NO: 984 - protein, 58 aa, synthetic construct
KTKNVVCNEELTELAEAKVLANRELDKYGVSDYHKNLINNAKTVEGVKDLQAQVVESA

SEQ ID NO: 985 - protein, 69 aa, synthetic construct
MKAIFVLNAQHDEAVDANSALAKAKVLANRELDKYGVSDYYKKLVNNAKTVEGVKDLQAQVVESAKKAR

SEQ ID NO: 986 - protein, 62 aa, synthetic construct
KTKNVVCNEELTALAKAKVLANRELDKYGVSDYYKKLVNKAKAAETVATLQAQVVESAKKAR

SEQ ID NO: 987 - protein, 15 aa, synthetic construct
  VARIANT 1..15: note = This sequence may encompass 1-5 GGS repeating units
GGSGGSGGSGGSGGS

SEQ ID NO: 988 - protein, 15 aa, synthetic construct
  VARIANT 1..15: note = This sequence may encompass 1-5 SSG repeating units
SSGSSGSSGSSGSSG

SEQ ID NO: 989 - protein, 5 aa, synthetic construct
ARVCF

SEQ ID NO: 990 - protein, 13 aa, synthetic construct
  SITE 5: note = Phosphotyrosine
ETDYYRKGGKGLL

SEQ ID NO: 991 - protein, 4 aa, synthetic construct
AAPF

SEQ ID NO: 992 - protein, 5 aa, synthetic construct
  SITE 1: note = Acetylated residue
  SITE 4: note = Phosphothreonine
  SITE 5: note = Amidated residue
LHSTA

SEQ ID NO: 993 - protein, 7 aa, synthetic construct
ALDKWNQ

SEQ ID NO: 994 - protein, 5 aa, synthetic construct
PYVNV

SEQ ID NO: 995 - protein, 10 aa, synthetic construct
ANSRHPTSII

SEQ ID NO: 996 - protein, 7 aa, synthetic construct
EPLVLHE

SEQ ID NO: 997 - protein, 6 aa, synthetic construct
  SITE 1: note = N-alpha-acetyl-L-thialysine
  SITE 2: note = Phosphotyrosine
XYVNVP

SEQ ID NO: 998 - protein, 11 aa, synthetic construct
PTGSSSTNPFR

SEQ ID NO: 999 - protein, 11 aa, synthetic construct
RVIPYFVPLNR

SEQ ID NO: 1000 - protein, 10 aa, synthetic construct
  SITE 7: note = Acetylated residue
ANSRWPKSII

SEQ ID NO: 1001 - protein, 10 aa, synthetic construct
ANSRWPTSIL

SEQ ID NO: 1002 - protein, 11 aa, synthetic construct
  SITE 11: note = Iodotyrosine
PPPPPPPPPPY

SEQ ID NO: 1003 - protein, 9 aa, synthetic construct
  SITE 1: note = Acetylated residue
  SITE 5: note = Phosphotyrosine
  SITE 9: note = Amidated residue
ELEFYMDYE

SEQ ID NO: 1004 - protein, 10 aa, synthetic construct
  SITE 5: note = Phosphotyrosine
TEGQYQPQPA

SEQ ID NO: 1005 - protein, 13 aa, synthetic construct
ELLELDKWASLWN

SEQ ID NO: 1006 - protein, 6 aa, synthetic construct
VFFAED

SEQ ID NO: 1007 - protein, 11 aa, synthetic construct
TEGQPYQPQPA

SEQ ID NO: 1008 - protein, 5 aa, unidentified
GGGGS

SEQ ID NO: 1009 - protein, 6 aa, synthetic construct
ENLYFS

SEQ ID NO: 1010 - protein, 4 aa, unidentified
WRPW

SEQ ID NO: 1011 - protein, 6 aa, unidentified
  SITE 2: note = Phosphoserine
  SITE 5: note = Phosphoserine
YSPTSS

SEQ ID NO: 1012 - protein, 14 aa, unidentified
ARKRRRHPSGPPTA

SEQ ID NO: 1013 - protein, 9 aa, unidentified
  SITE 5: note = 2-amino-3-(4-phosphonomethyl-phenyl)-propionic acid
PEGDXEEVL

SEQ ID NO: 1014 - protein, 9 aa, unidentified
RRRRRRRRR

SEQ ID NO: 1015 - protein, 7 aa, unidentified
RRRRRRR

SEQ ID NO: 1016 - protein, 21 aa, unidentified
TRSSRAGLQFPVGRVHRLLRK

SEQ ID NO: 1017 - protein, 16 aa, unidentified
RKKRRRESRKKRRRES

SEQ ID NO: 1018 - protein, 17 aa, unidentified
GRPRESGKKRKRKRLKP

SEQ ID NO: 1019 - protein, 15 aa, unidentified
GKRKKKGKLGKKRDP

SEQ ID NO: 1020 - protein, 17 aa, unidentified
GKRKKKGKLGKKRPRSR

SEQ ID NO: 1021 - protein, 18 aa, unidentified
RKKRRRESRRARRSPRHL

SEQ ID NO: 1022 - protein, 19 aa, unidentified
SRRARRSPRESGKKRKRKR

SEQ ID NO: 1023 - protein, 19 aa, unidentified
VKRGLKLRHVRPRVTRMDV

SEQ ID NO: 1024 - protein, 18 aa, unidentified
VKRGLKLRHVRPRVTRDV

SEQ ID NO: 1025 - protein, 14 aa, unidentified
SRRARRSPRHLGSG

SEQ ID NO: 1026 - protein, 16 aa, unidentified
LRRERQSRLRRERQSR

SEQ ID NO: 1027 - protein, 22 aa, unidentified
GAYDLRRRERQSRLRRRERQSR

SEQ ID NO: 1028 - protein, 30 aa, unidentified
WEAALAEALAEALAEHLAEALAEALEALAA

SEQ ID NO: 1029 - protein, 21 aa, unidentified
KGSWYSMRKMSMKIRPFFPQQ

SEQ ID NO: 1030 - protein, 20 aa, unidentified
KTRYYSMKKTTMKIIPFNRL

SEQ ID NO: 1031 - protein, 20 aa, unidentified
RGADYSLRAVRMKIRPLVTQ

SEQ ID NO: 1032 - protein, 24 aa, unidentified
LGTYTQDFNKFHTFPQTAIGVGAP

SEQ ID NO: 1033 - protein, 12 aa, unidentified
TSPLNIHNGQKL

SEQ ID NO: 1034 - protein, 12 aa, unidentified
NSAAFEDLRVLS

SEQ ID NO: 1035 - protein, 30 aa, unidentified
WEAKLAKALAKALAKHLAKALAKALKACEA

SEQ ID NO: 1036 - protein, 16 aa, unidentified
AAVALLPAVLLALLAP

SEQ ID NO: 1037 - protein, 10 aa, unidentified
VPMLKPMLKE

SEQ ID NO: 1038 - protein, 18 aa, unidentified
KLALKLALKALKAALKLA

SEQ ID NO: 1039 - protein, 27 aa, unidentified
GALFLGFLGAAGSTMGAWSQPKKKRKV

SEQ ID NO: 1040 - protein, 16 aa, unidentified
AAVALLPAVLLALLAP

SEQ ID NO: 1041 - protein, 9 aa, unidentified
VQRKRQKLM

SEQ ID NO: 1042 - protein, 16 aa, unidentified
RVIRVWFQNKRCKDKK

SEQ ID NO: 1043 - protein, 28 aa, unidentified
MANLGYWLLALFVTMWTDVGLCKKRPKP

SEQ ID NO: 1044 - protein, 18 aa, unidentified
LLIILRRRIRKQAHAHSK

SEQ ID NO: 1045 - protein, 18 aa, unidentified
VRLPPPVRLPPPVRLPPP

SEQ ID NO: 1046 - protein, 7 aa, unidentified
PKKKRKV

SEQ ID NO: 1047 - protein, 18 aa, unidentified
RGGRLSYSRRRFSTSTGR

SEQ ID NO: 1048 - protein, 10 aa, unidentified
RRLSYSRRRF

SEQ ID NO: 1049 - protein, 17 aa, unidentified
AWSFRVSYRGISYRRSR

SEQ ID NO: 1050 - protein, 14 aa, unidentified
YGRKKRRQRRRPPQ

SEQ ID NO: 1051 - protein, 11 aa, unidentified
YGRKKRRQRRR

SEQ ID NO: 1052 - protein, 9 aa, unidentified
RKKRRQRRR

SEQ ID NO: 1053 - protein, 16 aa, unidentified
RQIKIWFQNRRMKWKK

SEQ ID NO: 1054 - protein, 7 aa, unidentified
RRMKWKK

SEQ ID NO: 1055 - protein, 21 aa, unidentified
KETWWETWWTEWSQPKKKRKV

SEQ ID NO: 1056 - protein, 15 aa, unidentified
SDLWEMMMVSLACQY

SEQ ID NO: 1057 - protein, 27 aa, unidentified
GWTLNSAGYLLGKINLKALAALAKKIL

SEQ ID NO: 1058 - protein, 21 aa, unidentified
AGYLLGKINLKALAALAKKIL

SEQ ID NO: 1059 - protein, 12 aa, unidentified
GWTLNSAGYLLG

SEQ ID NO: 1060 - protein, 14 aa, unidentified
INLKALAALAKKIL

SEQ ID NO: 1061 - protein, 34 aa, unidentified
DAATATRGRSAASRPTERPRAPARSASRPRRPVD

SEQ ID NO: 1062 - protein, 26 aa, unidentified
DPKGDPKGVTVTVTVTVTGKGDPKPD

SEQ ID NO: 1063 - protein, 5 aa, unidentified
  MOD_RES 1: note = X is 2',6'-dimethyltyrosine
XDRFK

SEQ ID NO: 1064 - protein, 8 aa, unidentified
SKKKKIKV

SEQ ID NO: 1065 - protein, 8 aa, unidentified
GRKRKKRT

(19) United States
(12) Patent Application Publication
(10) Pub. No.: US 2025/0387450 A1
(43) Pub. Date: Dec. 25, 2025

(54) PROTEIN SCAFFOLDS FOR DISORDERED REGIONS

(71) Applicant: Aikium Inc., Oakland, CA (US)

(72) Inventors: Eswar IYER, Oakland, CA (US); Shankar SHASTRY, Pleasanton, CA (US); Venkatesh Pranesh MYSORE, Castro Valley, CA (US)

(21) Appl. No.: 19/182,278

(22) Filed: Apr. 17, 2025

Related U.S. Application Data
(60) Provisional application No. 63/635,567, filed on Apr. 17, 2024.

(51) Int. Cl. A61K 38/17; A61K 38/45; C07K 14/74; C12N 9/10

(52) U.S. Cl. CPC A61K 38/1774; A61K 38/45; C07K 14/70539; C12N 9/104; C07K 2319/30; C07K 2319/50

(57) ABSTRACT

The disclosure provides proteins that can be used as scaffolds to bind a specific target, including specific proteins or peptides of interest, as well as fusion proteins with the scaffolds. Such proteins or peptides of interest may include disordered regions. The invention also relates to various uses of the scaffold, including diagnostics, therapeutics, detection reagents, and vaccines. The protein scaffolds can be tailored to specifically bind a target using the methods described herein.

### CROSS-REFERENCE TO RELATED APPLICATIONS

This application claims the benefit of U.S. Provisional Patent Application No. 63/635,567 filed Apr. 17, 2024, the entire contents of which are incorporated herein by reference.

### INCORPORATION BY REFERENCE OF SEQUENCE LISTING

The instant application contains a Sequence Listing, which has been submitted via Patent Center. The Sequence Listing titled 211977-704102_US_SL.xml, which was created on Apr. 17, 2025 and is 1,236,022 bytes in size, is hereby incorporated by reference in its entirety.

### FIELD

The present invention relates to protein scaffolds that specifically bind to a disordered region of a target protein, and methods for making, screening, and using such protein scaffolds.

### BACKGROUND

The targeting of proteins based on their three-dimensional structure, determined experimentally or modeled computationally, has become one of the foundational techniques in modern drug discovery. This has led to a great deal of focus on the secondary structures (e.g., alpha-helices or beta-sheets) for targeting interaction with the protein based on those secondary structures. However, while most proteins or segments thereof fold in a defined three-dimensional structure, studies over the last few decades have discovered that there are numerous segments of polypeptides that do not fold into a defined structure. In fact, some proteins can still carry out their function in an unstructured/disordered state. Utilization of these disordered regions of a target protein have been underutilized in drug development. Thus, there is a need for targeted protein discovery that focuses on these disordered regions. The embodiments described herein satisfy this need and provide related advantages.

### SUMMARY

The present disclosure provides engineered scaffold proteins, as well as fusion proteins comprising such scaffolds, that bind a specific target protein or peptide of interest, as well as various uses of the engineered scaffold proteins and fusion proteins.

Provided herein is an engineered scaffold protein that can specifically bind to a disordered region in a target peptide; wherein the engineered scaffold protein comprises: one or more binding domain comprising one or more binding units and one or more hinge units, wherein: the one or more binding units is capable of binding to the disordered region of the target peptide and comprises one or more amino acid alterations relative to a wildtype (WT) counterpart, and the one or more hinge units is capable of stabilizing the structure of the binding domain for binding; wherein binding activity of the target peptide by the engineered scaffold protein is increased relative to a WT counterpart of the engineered scaffold. In some embodiments, the engineered scaffold protein comprises one or more molecular transporters. In some embodiments, the molecular transporter is selected from the group consisting of cell penetrating peptides (CPPs) including cell penetrating poly(disulfides) (CPDs), protein-transduction domains (PTD) or membrane-translocation sequences (MTS), lipids, liposomes including cationic liposomes (e.g., as part of a cationic lipid and neutral co-lipid system), lipid nanoparticles, steroids, polyamines, nanotubes, nanoparticles including porous silicon nanoparticles (Si NPs) and polymeric nanoparticles including PEG-hydroxyethylene disulfide polymers), dendrimers (including guanidinium terminated dendrimers, peptoids (including N-substituted glycines), oligogcarbamates, HIV-binding peptides, HIV-1 Tat (HIV), Tat-derived peptides, Penetratin, VP22 derived or analog peptides, Pestivirus Erns, HSV, VP22 (Herpes simplex), MAP, KALA, PpT620, prolin-rich peptides, arginine-rich peptides, lysine-rich peptides, MPG-peptide(s), Pep-1, L-oligomers, Calcitonin peptide(s), Antennapedia-derived peptides including from Drosophila antennapedia, pAntp, pIsl, FGF, Lactoferrin, Transportan, Buforin-2, Bac715-24, SynB, SynB (1), pVEC, hCT-derived peptides, SAP, histones, modified or phosphorothioated single-stranded DNA (ssDNA), polymer-based materials including polyethylene glycol, polyethyleneimine, and polyboronic acids, protein nanocarriers including a Hex carrier, transmembrane antibodies, nanocapsules (NCs) including PEG coated hyaluronic acid nanoparticles (NPs), polymer NPs with glucose head groups, protein transduction domain mimics, anionic peptides, pyridylthiourea-modified polyethyleneimine (PEI), mutated lycotoxin, Sesbania mosaic virus like particles (VLPs), PEG- and arginine-capped lipids, microinjection and electroporation, poly(lactide-co-glycolic acid) (PLGA) NPs.

Also provided herein are engineered scaffold proteins wherein the increase of binding activity comprises an increase in binding frequency, binding rapidity, binding duration, binding affinity, or any combination thereof. Also provided herein are engineered scaffold proteins, wherein the engineered scaffold protein comprises: decreased immunogenicity relative to a corresponding WT; increased solubility relative to a corresponding WT; increased stability relative to a corresponding WT; increased or decreased hydrophobicity relative to a corresponding WT; increased or decreased hydrophilicity relative to a corresponding WT; increased or decreased surface charge relative to a corresponding WT; or any combination of the foregoing. Also provided herein are engineered scaffold proteins that can specifically bind to disordered regions in target peptides, wherein the disordered region is located in an internal loop, C-terminal tail or N-terminal tail of a target peptide. Also provided herein are engineered scaffold proteins that can specifically bind to disordered regions in target peptides, wherein the disordered region in the target peptide comprises a linear epitope which is bound by the binding unit. Also provided herein are engineered scaffold proteins that can specifically bind to disordered regions in target peptides, wherein the linear epitope comprises about 4 to about 40, about 4 to about 30, or about 4 to about 25 amino acids in length. Also provided herein are engineered scaffold proteins that can specifically bind to disordered regions in target peptides, wherein the target peptide is comprised in a polypeptide, protein, or protein complex. Also provided herein are engineered scaffold proteins that can specifically bind to disordered regions in target peptides, wherein the target peptide is comprised in a polypeptide, protein, or protein complex, and wherein the polypeptide, protein, or one protein within the protein complex is greater than about 30 amino acids in length. Also provided herein are engineered scaffold proteins that can specifically bind to disordered regions in target peptides, wherein the target peptide is comprised in a polypeptide, protein, or protein complex, wherein the polypeptide, protein, or one protein within the protein complex is greater than about 100 daltons in weight. Also provided herein are engineered scaffold proteins that can specifically bind to disordered regions in target peptides, wherein the target peptide is comprised in an extracellular protein. Also provided herein are engineered scaffold proteins that can specifically bind to disordered regions in target peptides, wherein the target peptide is comprised in a membrane protein. Also provided herein are engineered scaffold proteins that can specifically bind to disordered regions in target peptides, wherein the target peptide is comprised in an extracellular protein or a membrane protein comprising a receptor, an ion channel, or a secreted protein. Also provided herein are engineered scaffold proteins that can specifically bind to disordered regions in target peptides, wherein the target peptide is comprised in extracellular protein comprising GPCR. Also provided herein are engineered scaffold proteins, wherein the binding unit comprises an elongated configuration. Also provided herein are engineered scaffold proteins, wherein upon being folded in its tertiary conformation, the binding unit comprises a three-dimensional conformation comprising one or more amino acids which are anti-sense to one or more amino acids of the disordered region of the target peptide as determined by sense-antisense amino acid pairing. Also provided herein are engineered scaffold proteins, wherein the engineered scaffold protein comprises two binding units. Also provided herein are engineered scaffold proteins, wherein each of the one or more binding units comprises about 40 to about 200 amino acids, about 60 to about 150 amino acids, or about 80 to about 100 amino acids. Also provided herein are engineered scaffold proteins, wherein the one or more binding units each comprises one or more helices comprising alpha helices, 3.10 helices, and/or pi helices. Also provided herein are engineered scaffold proteins, wherein the one or more binding units each comprises one or more alpha helices. Also provided herein are engineered scaffold proteins, wherein the one or more binding units each comprises 2, 3, 4, 5, 6, 7, 8, 9 or 10 or more amino acid alterations relative to a WT counterpart. Also provided herein are engineered scaffold proteins, wherein each of the one or more hinge units comprises about 40 to about 200 amino acids, about 60 to about 150 amino acids, or about 80 to about 100 amino acids. Also provided herein are engineered scaffold proteins, wherein the one or more hinge units comprises a concave, or partially concave, configuration. Also provided herein are engineered scaffold proteins, wherein the one or more hinge units comprise a partially flexible conformation that can conform to the disordered region of the target peptide when the disordered region is bound by the engineered scaffold protein. Also provided herein are engineered scaffold proteins, wherein the one or more hinge units comprises one or more beta sheet strands, linear peptides, covalent interactions, non-covalent interactions, chemical agents, or any combination thereof. Also provided herein are engineered scaffold proteins, wherein the hinge unit comprises about 3 to about 12 beta sheet protein strands, or about 6 to about 10 beta sheet protein strands which form one or more beta sheets. Also provided herein are engineered scaffold proteins, wherein the hinge unit comprises one or two beta sheets. Also provided herein are engineered scaffold proteins, wherein the one or more hinge units are covalently attached or linked by one or more linking units to the one or more binding units, or combinations thereof. Also provided herein are engineered scaffold proteins, wherein the one or more linking units are one or more linkers. Also provided herein are engineered scaffold proteins, wherein the one or more linkers comprise a peptide linker. Also provided herein are engineered scaffold proteins, wherein the one or more binding units and one or more hinge units are connected as a monomer. Also provided herein are engineered scaffold proteins, wherein the one or more hinge unit is attached to the N terminus or C terminus of the one or more binding units. Also provided herein are engineered scaffold proteins, wherein the engineered scaffold protein comprises two binding units, and one hinge unit, wherein the first binding unit is attached to the N terminus of the hinge unit and the second binding unit is attached to the C terminus of the hinge unit. Also provided herein are engineered scaffold proteins, wherein the engineered scaffold protein comprises two binding units, and one hinge unit, wherein the first binding unit is attached to the N terminus of the second binding unit and the hinge unit is attached to the C terminus of the second binding unit. Also provided herein are engineered scaffold proteins, wherein the two or more binding units are in an anti-parallel configuration. Also provided herein are engineered scaffold proteins, wherein the two or more binding units are semi-symmetrical. Also provided herein are engineered scaffold proteins, wherein the one or more binding units and one or more hinge units are multimeric wherein the one or more binding units and one or more hinge units form as a binding domain in the presence of the target peptide. Also provided herein are engineered scaffold proteins, wherein the binding domain is derived from any one of the scaffold proteins set forth in TABLE 1. Also provided herein are engineered scaffold proteins, wherein the binding domain is in a binding groove architecture. Also provided herein are engineered scaffold proteins, wherein the binding domain comprises a Clamshell structure. Also provided herein are engineered scaffold proteins, wherein the engineered scaffold protein further comprises one or more immunoglobulin units. Also provided herein are engineered scaffold proteins, wherein the engineered scaffold protein comprises two immunoglobulin units. Also provided herein are engineered scaffold proteins, wherein the one or more immunoglobulin units is covalently attached or attached by a linking unit to the binding domain, hinge unit, binding unit, or combination thereof. Also provided herein are engineered scaffold proteins, wherein the engineered scaffold protein does not comprise an immunoglobulin unit. Also provided herein are engineered scaffold proteins, wherein the engineered scaffold protein comprises an isoelectric point of 3.5 to 9, 4 to 8.5, or 4.5 to 8, when measured in an electrophoresis assay. Also provided herein are engineered scaffold proteins, wherein the binding unit comprises an amino acid sequence that is at least 65% identical to any one of the sequences in TABLE 4. Also provided herein are engineered scaffold proteins, wherein the hinge unit comprises an amino acid sequence that is at least 65% identical to any one of the sequences in TABLE 5. Also provided herein are engineered scaffold proteins, wherein the disordered region in a target peptide comprises an amino acid sequence selected from the list of bound peptide sequences listed in TABLE 3 or variant thereof having one, two, three, four, five, six, seven, eight, nine, or ten amino acid alterations, or more. Also provided herein are engineered scaffold proteins, wherein said engineered protein scaffold is conjugated to a heterologous agent for extending the half-life of the engineered scaffold. Also provided herein are engineered scaffold proteins, wherein said heterologous agent is selected from the group consisting of polyethylene glycol (PEG), human serum albumin (HSA), and a variant Fc region of an antibody. Also provided herein are engineered scaffold proteins, wherein the half-life of the engineered protein scaffold is extended relative to a WT counterpart. Also provided herein are engineered scaffold proteins, wherein the disordered region in a target peptide comprises an amino acid sequence selected from the list of bound peptide sequences listed in TABLE 7 or variant thereof having one, two, three, four, five, six, seven, eight, nine, or ten amino acid alterations, or more. Also provided herein are engineered scaffold proteins, wherein the engineered scaffold protein further comprises one or more epitope masking units. Also provided herein are engineered scaffold proteins, wherein the epitope masking unit interacts with the binding unit of the engineered scaffold protein. Also provided herein are engineered scaffold proteins, wherein the epitope masking unit is linked to the N terminal, C terminal or in an intermediary loop of the engineered scaffold protein. Also provided herein are engineered scaffold proteins, wherein the epitope masking unit is linked to the N terminal, C terminal or in an intermediary loop of the engineered scaffold protein. Also provided herein are engineered scaffold proteins, wherein the epitope masking unit, wherein the epitope masking unit can partially occlude the binding unit, support the tertiary conformation of the engineered scaffold protein, improve the immunogenicity of the engineered scaffold protein, or any combination thereof. Also provided herein are engineered scaffold proteins, epitope masking unit is displaced by the disordered region of the target peptide upon binding by the binding unit. Also provided herein are engineered scaffold proteins, wherein the engineered scaffold protein comprises a functional classification selected from the list of functional classification of human protein scaffolds listed in TABLE 2. Also provided herein are engineered scaffold proteins, wherein the engineered scaffold protein comprises an amino acid sequence that is at least 65% identical to any one of the sequences set forth in TABLE 6. Also provided herein are engineered scaffold proteins, wherein the engineered scaffold protein comprises an amino acid sequence that is at least 75% identical to any one of the sequences set forth in TABLE 6. Also provided herein are engineered scaffold proteins, wherein the engineered scaffold protein comprises an amino acid sequence that is at least 80% identical to any one of the sequences set forth in TABLE 6. Also provided herein are engineered scaffold proteins, wherein the engineered scaffold protein comprises an amino acid sequence that is at least 85% identical to any one of the sequences set forth in TABLE 6. Also provided herein are engineered scaffold proteins, wherein the engineered scaffold protein comprises an amino acid sequence that is at least 95% identical to any one of the sequences set forth in TABLE 6. Also provided herein are engineered scaffold proteins, wherein the engineered scaffold protein comprises an amino acid sequence that is at least 97% identical to any one of the sequences set forth in TABLE 6. Also provided herein are engineered scaffold proteins, wherein the engineered scaffold protein comprises an amino acid sequence that is at least 98% identical to any one of the sequences set forth in TABLE 6. Also provided herein are engineered scaffold proteins, wherein the engineered scaffold protein comprises an amino acid sequence that is at least 99% identical to any one of the sequences set forth in TABLE 6. Also provided herein are engineered scaffold proteins, wherein the engineered scaffold protein comprises an amino acid sequence that is identical to any one of the sequences set forth in TABLE 6.

Provided herein are fusion proteins comprising an engineered scaffold provided herein and a fusion partner. Also provided herein are fusion proteins, wherein the fusion partner is an enzyme. Also provided herein are fusion proteins, wherein the enzyme catalyzes ubiquitination, post-translational modification, proteolytic cleavage, dephosphorylation, trans-cis isomerization, protein chaperone activity, nucleic acid modifying proteins, ATPase or GTPase activity. Also provided herein are fusion proteins, wherein the fusion partner selectively binds to a specific region on a target protein. Also provided herein are fusion proteins, wherein the specific region on a target protein is not a disordered region of said target protein.

Provided herein are methods of binding a disordered region of a target protein comprising contacting a target protein comprising said disordered region with the engineered scaffold protein provided herein.

Provided herein are methods of binding a disordered region of a target protein comprising contacting a target protein comprising said disordered region with the fusion protein provided herein.

Provided herein are methods of changing the conformation of a protein comprising a target peptide, the method comprising contacting said target peptide with the engineered scaffold protein provided herein.

Provided herein are methods of changing the conformation of a protein comprising a target peptide, the method comprising contacting said target peptide with the fusion protein provided herein.

Provided herein are methods of treating a disease comprising administering to a subject an effective amount of the engineered scaffold protein provided herein.

Provided herein are methods of treating a disease comprising administering to a subject an effective amount of the fusion protein provided herein.

Provided herein are methods of inducing an immune response in a subject in need thereof, said method comprising administering to said subject the engineered scaffold protein provided herein.

Provided herein are methods of inducing an immune response in a subject in need thereof, said method comprising administering to said subject the fusion protein provided herein.

Provided herein are methods of detecting a target protein comprising contacting a sample suspected of comprising a target protein with at least the engineered scaffold protein provided herein.

Provided herein are methods of detecting a target protein comprising contacting a sample suspected of comprising a target protein with at least the fusion protein provided herein.

Provided herein are kits comprising at least the engineered scaffold protein provided herein.

Provided herein are kits comprising at least the fusion protein provided herein.

Provided herein are devices comprising at least the engineered scaffold protein provided herein.

Provided herein are devices comprising at least the fusion protein provided herein.

Provided herein are methods of detecting a compound in a sample, said method comprising contacting said sample with the engineered scaffold protein provided herein.

Provided herein are methods of detecting a compound in a sample, said method comprising contacting said sample with the fusion protein provided herein.

Provided herein are pharmaceutical compositions comprising the engineered scaffold protein provided herein and a pharmaceutically acceptable excipient.

Provided herein are pharmaceutical compositions comprising the fusion protein provided herein and a pharmaceutically acceptable excipient.

Provided herein are methods of preventing, treating, or managing a disease in a subject in need thereof by administering an effective amount of the pharmaceutical composition provided herein.

Provided herein are methods of preventing, treating, or managing a disease in a subject in need thereof by administering an effective amount of the pharmaceutical composition provided herein. Provided herein are isolated nucleic acid molecules encoding the engineered scaffold protein provided herein.

Provided herein are isolated nucleic acid molecules encoding the fusion protein provided herein.

Provided herein are expression vectors operably linked to the nucleic acid provided herein.

Provided herein are expression vectors operably linked to the nucleic acid provided herein.

Provided herein are host cells comprising the vector provided herein.

Provided herein are host cells comprising the vector provided herein.

Provided herein are polypeptide display libraries comprising the engineered scaffold protein provided herein.

Provided herein are polypeptide display libraries comprising the fusion protein provided herein. Also provided herein are polypeptide display libraries, wherein said engineered scaffold protein is displayed on the surface of a virus, or yeast, or displayed as a ribsome or RNA conjugated protein molecule. Also provided herein are polypeptide display libraries, wherein said fusion protein is displayed on the surface of a virus, or yeast, or displayed as a ribsome or RNA conjugated protein molecule.

Provided herein are collections of isolated nucleic acid molecules encoding the libraries provided herein.

Provided herein are methods of obtaining an engineered scaffold that binds to a target, said method comprising (a) contacting a target ligand with a library provided herein under conditions that allow an engineered scaffold protein: target ligand complex to form, and (b) obtaining from the complex, the scaffold that binds the target ligand.

Provided herein are methods of obtaining a fusion protein that binds to a target, said method comprising (a) contacting a target ligand with a fusion protein library provided herein under conditions that allow a fusion protein: target ligand complex to form, and (b) obtaining from the complex, the fusion protein that binds the target ligand.

Provided herein are methods of obtaining at least two engineered scaffold proteins that bind to a target, said method comprising (a) contacting a target ligand with a library provided herein under conditions that allow an engineered scaffold: target ligand complex to form, (b) engaging said complex with a crosslinking agent wherein the crosslinking of said complex elicits a detectable response and (c) obtaining from the complex, said engineered scaffold proteins that bind the target. Also provided herein are methods of obtaining at least two engineered scaffold proteins that bind to a target, wherein said engineered scaffold proteins recognize the same epitope. Also provided herein are methods of obtaining at least two engineered scaffold proteins that bind to a target, wherein said engineered scaffold proteins recognize distinct epitopes. Also provided herein are methods of obtaining at least two engineered scaffold proteins that bind to a target, wherein said crosslinking agent is selected from the group consisting of an antibody, an antibody fragment, a binding peptide, or an epitope tag.

Provided herein are methods of obtaining at least two fusion proteins that bind to a target, said method comprising (a) contacting a target ligand with a fusion protein library provided herein under conditions that allow a fusion protein: target ligand complex to form, (b) engaging said complex with a crosslinking agent wherein the crosslinking of said complex elicits a detectable response and (c) obtaining from the complex, said fusion proteins that bind the target. Also provided herein are methods of obtaining at least two fusion proteins that bind to a target, wherein said fusion proteins recognize the same epitope. Also provided herein are methods of obtaining at least two fusion proteins that bind to a target, wherein said fusion proteins recognize distinct epitopes. Also provided herein are methods of obtaining at least two fusion proteins that bind to a target, wherein said crosslinking agent is selected from the group consisting of an antibody, an antibody fragment, a binding peptide, or an epitope tag.

Provided herein are methods of generating a sequence of an engineered scaffold protein that can bind to a disordered region of a target peptide, the method comprising: selecting one or more scaffold protein sequences; evaluating the one or more scaffold protein sequences for one or more desired characteristics comprising: ligand binding, immunogenicity, binding selectivity, binding frequency, binding speed, binding affinity, binding duration, function or biological activity, resistance to proteolytic cleavage, solubility, stability, half-life, or any combination thereof; engineering an amino acid sequence of an engineered scaffold protein based on the evaluation of the one or more scaffold protein sequences; wherein the engineered scaffold protein is predicted to have enhancement or improvement of the one or more desired characteristics relative to the one or more scaffold protein counterpart. Also provided herein are methods of generating a sequence of an engineered scaffold protein that can bind to a disordered region of a target peptide, wherein engineering an amino acid sequence of an engineered scaffold protein comprises engineering the amino acid sequence to bind to a linear epitope of a target peptide. Also provided herein are methods of generating a sequence of an engineered scaffold protein that can bind to a disordered region of a target peptide, wherein evaluating the one or more scaffold protein sequences for one or more desired characteristics comprises weighing one or more factors of the one or more scaffold protein sequences relevant to one or more desired characteristics, wherein the one or more weighed factors comprises: three-dimensional conformation, protein domain(s), amino acid sequence, amino acid charge, amino acid polarity, amino acid hydrophobicity/hydrophilicity, amino acid acidity/baseness, or any combination thereof. Also provided herein are methods of generating a sequence of an engineered scaffold protein that can bind to a disordered region of a target peptide, wherein weighing one or more factors comprises: assigning a value to the one or more weighed factors based on an estimated probability of enhancing one or more desired characteristics; assigning a value to the one or more weighed factors, measuring the deviation of said value relative to a target value or to a value assigned to said factor of a second scaffold protein; or both of the foregoing. Also provided herein are methods of generating a sequence of an engineered scaffold protein that can bind to a disordered region of a target peptide, wherein the method further comprises predicting: whether an engineered amino acid sequence represents an engineered scaffold protein exhibiting an enhanced characteristic; whether one or more alteration of the one or more weighed factors improves said value relative to a target value or threshold or to a value assigned to such a factor of a second scaffold protein; or both of the foregoing. Also provided herein are methods of generating a sequence of an engineered scaffold protein that can bind to a disordered region of a target peptide, wherein the one or more alteration comprises one or more amino acid alteration. Also provided herein are methods of generating a sequence of an engineered scaffold protein that can bind to a disordered region of a target peptide, wherein the engineered amino acid sequence of the engineered scaffold protein comprises one or more alterations relative to a counterpart scaffold protein. Also provided herein are methods of generating a sequence of an engineered scaffold protein that can bind to a disordered region of a target peptide, wherein evaluating the one or more scaffold protein sequences comprises manually evaluating the amino acid sequences of the one or more scaffold protein sequences or evaluating the amino acid sequences of the one or more scaffold protein sequences in a machine learning model. Also provided herein are methods of generating a sequence of an engineered scaffold protein that can bind to a disordered region of a target peptide wherein the method further comprises a second or more iteration of: evaluating one more desired characteristics of the engineered amino acid sequence of the engineered scaffold protein; weighing one or more weighed factors of the engineered amino acid sequence of the engineered scaffold protein; or both of the foregoing; and informing further engineering of the amino acid sequences of the of engineered scaffold proteins. Also provided herein are methods of generating a sequence of an engineered scaffold protein that can bind to a disordered region of a target peptide, wherein the method comprises generating the engineered scaffold protein. Also provided herein are methods of generating a sequence of an engineered scaffold protein that can bind to a disordered region of a target peptide, wherein the method comprises assaying the engineered scaffold protein. Also provided herein are methods of generating a sequence of an engineered scaffold protein that can bind to a disordered region of a target peptide, wherein assaying comprises one or more in vitro assay or in vivo assay. Also provided herein are methods of generating a sequence of an engineered scaffold protein that can bind to a disordered region of a target peptide, wherein assaying comprises in silico simulations and/or machine learning model simulations. Also provided herein are methods of generating a sequence of an engineered scaffold protein that can bind to a disordered region of a target peptide, wherein assaying the generated engineered scaffold protein comprises obtaining data and informing further generation of engineered scaffold proteins. In some embodiments, the methods herein comprise engineering the amino acid sequence of the engineered scaffold protein to target one or more post-translational modifications. In some embodiments, the methods comprise engineering the amino acid sequence of the engineered scaffold protein to target a therapeutic peptide. In some embodiments, there methods comprise engineering the amino acid sequence of the engineered scaffold protein to modulate one or more cell signalling pathways. Provided herein are systems comprising instructions capable of performing the methods provided herein.

Provided herein are engineered scaffold proteins comprising an amino acid sequence generated by the methods provided herein or the systems provided herein.

Provided herein are compositions comprising the engineered scaffold protein provided herein, or the fusion protein provided herein, and a therapeutic partner. Also provided herein are compositions, wherein the therapeutic partner is attached to the engineered scaffold protein or the fusion protein, wherein the therapeutic partner is covalently bonded, non-covalently bonded, fused, conjugated, and/or linked to the engineered scaffold protein or the fusion protein. Also provided herein are compositions, wherein the therapeutic partner comprises one or more of anti-cancer agents, anti-inflammatories, anti-bacterials, anti-virals, cytokines, toxins, enzymes, neuroprotective agents, soluble factor traps, or any combination thereof. Also provided herein are compositions, wherein the therapeutic partner can be unattached from the engineered scaffold protein or the fusion protein upon binding with the target peptide. Also provided herein are compositions, wherein the engineered scaffold protein or the fusion protein can be tissue or cell specific. Also provided herein are compositions, wherein the therapeutic partner can be unattached from the engineered scaffold protein or the fusion protein upon reaching the specific cell or tissue. Provided herein are methods of treating a disease or disorder comprising administering to a subject in need thereof the composition provided herein.

Provided herein are methods of chimeric antigen receptor (CAR) T-cell therapy comprising administering to a subject in need thereof: the engineered scaffold protein provided herein; or the fusion protein provided herein.

In some embodiments, the engineered scaffold proteins herein comprise a detectable moiety. Provided herein are methods of detecting a disease in a subject in need thereof. In some embodiments, the method comprises: obtaining a sample from the subject, contacting the sample with one or more engineered scaffold proteins herein, and detecting the signal emitted from a detectable moiety.

Other features and advantages of the invention will be apparent from the detailed description, and from the claims.

### BRIEF DESCRIPTION OF THE DRAWINGS

FIG. 1 shows a schematic of an engineered scaffold or fusion protein thereof acting as an inhibitor on a target mutant protein having a disordered region. (A) An engineered scaffold or fusion protein thereof that masks the mutant region with high specificity and binds to the domain for protein C. (B) An interacting protein that may be wild-type or mutant, and whose interaction causes disease. (C) A protein that preferentially interacts with the engineered scaffold or fusion protein thereof (A) once it is bound to the disordered region and does not cause disease.

FIG. 2 shows a schematic of a disease target protein without an engineered scaffold or fusion protein thereof vs. with the engineered scaffold or fusion protein thereof acting as an inhibitor. The engineered scaffold or fusion protein thereof (A) masks the mutant region (B) with high specificity and inhibits the interaction.

FIG. 3 shows a schematic of an engineered scaffold or fusion protein thereof (A) modulating DNA/RNA binding via binding to the disordered region of a disease target, thereby rendering the DNA/RNA unable to bind to the target.

FIG. 4 shows a schematic of a macromolecule with a disordered region that is unable to bind to a target vs. a macromolecule that is able to bind to a target following binding of an engineered scaffold or fusion protein thereof (A). Binding of the engineered scaffold or fusion protein thereof to the disordered region modulates DNA/RNA binding.

FIG. 5 shows a schematic of a fusion protein having an engineered scaffold fused to a fusion partner that catalyzes ubiquitination. The scaffold-E3 ligase fusion binds to the disease target having a disordered region and ubiquitinates the target, marking it for degradation.

FIG. 6 shows a schematic of a fusion protein having an engineered scaffold fused to a fusion partner that possesses certain enzymatic activity. The engineered scaffold or fusion protein thereof (A) binds a disease target having a disordered region, and the enzyme portion of the fusion protein modulates biological activity of the target or other proteins in the vicinity of the binding. Such enzymatic activity includes post-translational modification, proteolytic cleavage, dephosphorylation, trans-cis isomerization, protein chaperone activity, catalyzation of nucleic acid modifying proteins, ATPase or GTPase activity, etc.

FIG. 7 shows a schematic of a functional protein (left), a misfolded protein having at least one disordered region (middle), and a restored functional protein having two ordered domains (right) following binding of a fusion protein having two engineered scaffold proteins (A) fused together or a fusion protein having at least one engineered scaffold protein and a chaperone protein. A functional protein and a resulting restored functional protein are also displayed for reference.

FIG. 8 shows a schematic of local protein editing by reversing post-translational modifications (PTMs) in a misfolded protein (center) following binding of a fusion protein having an engineered scaffold protein and a fusion partner having enzymatic activity (A). A functional protein (left) and a resulting restored functional protein (right) are also displayed for reference.

FIG. 9 shows a schematic of local protein editing by sequence specific degradation in a misfolded protein using a fusion protein having an engineered scaffold protein and a fusion partner having degradation activity (A) on the misfolded protein (center). A functional protein (left) and the resulting restored functional protein (right) are also displayed for reference.

FIG. 10 shows a schematic of various forms an engineered scaffold protein that can be used for therapeutic developments, including a fusion protein having an engineered scaffold protein and a fusion partner, a PEG conjugated engineered scaffold protein, and a multispecific scaffold construct.

FIG. 11 shows a diagram of an exemplary engineered scaffold protein engineering process.

FIG. 12 shows a diagram overview of an exemplary stepwise engineered scaffold protein optimization process.

FIG. 13 shows a diagram of an exemplary engineered scaffold protein development pipeline.

FIG. 14 shows a schematic of a fusion protein having an engineered scaffold fused to a fusion partner that comprises an Fc region of an antibody. The engineered scaffold binds a target protein having a disordered region that is expressed on the surface of a target cell or binds a target antigen having a disordered region, and the Fc portion of the fusion protein activates a cascade of events resulting in the killing of the target cell and/or destruction of the target antigen.

FIG. 15A shows a representative SDS PAGE gel of an engineered scaffold protein or fusion protein thereof which is ˜22 kDa and runs at the expected size on the PAGE gel. Engineered scaffold proteins range in size from ˜ 20 kDa to ˜ 50 kDa.

FIG. 15B shows a size exclusion chromatogram of an engineered scaffold protein, which shows that much of the protein generated runs as a monomer (tallest peak) on the column, while a smaller fraction is split between dimers and multimer aggregates. X-axis represents the volume (mL) and y-axis represents absorbance (Abs, mAu).

FIG. 16A shows a graph depicting the binding affinity between an engineered protein scaffold (S005, S007, S090) and no scaffold control and target, CD74. X-axis represents concentration of target (nM) and y-axis represents fluorescence (AU).

FIG. 16B shows a graph depicting the binding affinity between an engineered protein scaffold (S005, S007, S090) and no scaffold control and target, KAAG1. X-axis represents concentration of target (nM) and y-axis represents fluorescence (AU).

FIG. 16C shows a graph depicting the binding affinity between an engineered protein scaffold (S005, S007, S090) and no scaffold control and target, CysLT2. X-axis represents concentration of target (nM) and y-axis represents fluorescence (AU).

FIG. 16D shows a graph depicting the binding affinity between an engineered protein scaffold (S005, S007, S090) and no scaffold control and target, CRACM. X-axis represents concentration of target (nM) and y-axis represents fluorescence (AU).

FIG. 16E shows a graph depicting the binding affinity between an engineered protein scaffold (S005, S007, S090) and no scaffold control and target, Kv1.5. X-axis represents concentration of target (nM) and y-axis represents fluorescence (AU).

FIG. 17A shows a graph depicting the improvement in target binding after scaffold optimization, the points represent no scaffold control, and three versions of engineered scaffold proteins. X-axis represents concentration of target (nM) and y-axis represents fluorescence (AU).

FIG. 17B shows a bar graph depicting the improvement in affinity after scaffold optimization. Each bar represents no scaffold control, and three versions of engineered scaffold proteins, respectively and y-axis represents relative binding.

FIG. 17C shows a bar graph depicting the improvement in relative binding after scaffold optimization. Each bar represents no scaffold control, and three versions of engineered scaffold proteins, respectively and y-axis represents fold change.

FIG. 17D shows a bar graph depicting the improvement in specificity after scaffold optimization. Each bar represents an engineered scaffold protein, and y-axis represents fold change.

FIG. 18A shows a graph depicting the binding affinity between an engineered protein scaffold (S058, S065, S090) and no scaffold control and target, CD74. X-axis represents concentration of target (nM) and y-axis represents fluorescence (AU).

FIG. 18B shows a graph depicting the binding affinity between an engineered protein scaffold (S058, S065, S090) and no scaffold control and target, KAAG1. X-axis represents concentration of target (nM) and y-axis represents fluorescence (AU).

FIG. 18C shows a graph depicting the binding affinity between an engineered protein scaffold (S058, S065, S090) and no scaffold control and target, CysLT2r. X-axis represents concentration of target (nM) and y-axis represents fluorescence (AU).

FIG. 18D shows a graph depicting the binding affinity between an engineered protein scaffold (S058, S065, S090) and no scaffold control and target, CRACM. X-axis represents concentration of target (nM) and y-axis represents fluorescence (AU).

FIG. 18E shows a graph depicting the binding affinity between an engineered protein scaffold (S058, S065, S090) and no scaffold control and target, Kv1.5. X-axis represents concentration of target (nM) and y-axis represents fluorescence (AU).

FIG. 19A shows a graph depicting the binding affinity between an engineered protein scaffold (S090, S164, S171) and no scaffold control and target, CD74. X-axis represents concentration of target (nM) and y-axis represents fluorescence (AU).

FIG. 19B shows a graph depicting the binding affinity between an engineered protein scaffold (S090, S164, S171) and no scaffold control and target, KAAG1. X-axis represents concentration of target (nM) and y-axis represents fluorescence (AU).

FIG. 19C shows a graph depicting the binding affinity between an engineered protein scaffold (S090, S164, S171) and no scaffold control and target, CysLTR2. X-axis represents concentration of target (nM) and y-axis represents fluorescence (AU).

FIG. 19D shows a graph depicting the binding affinity between an engineered protein scaffold (S090, S164, S171) and no scaffold control and target, CRACM. X-axis represents concentration of target (nM) and y-axis represents fluorescence (AU).

FIG. 19E shows a graph depicting the binding affinity between an engineered protein scaffold (S090, S164, S171) and no scaffold control and target, CRACM. X-axis represents concentration of target (nM) and y-axis represents fluorescence (AU).

FIG. 20A shows a graph depicting the binding affinity between an engineered protein scaffold (S090, S177) and no scaffold control and target, MERKFMSLQPSISVSEMEPNG (SEQ ID NO: 927). X-axis represents concentration of target (nM) and y-axis represents fluorescence (AU).

FIG. 20A shows a graph depicting the binding affinity between an engineered protein scaffold (S090, S177) and no scaffold control and target, MERKFMSLQPSISVSEMEPNG (SEQ ID NO: 927). X-axis represents concentration of target (nM) and y-axis represents fluorescence (AU).

FIG. 20B shows a graph depicting the binding affinity between an engineered protein scaffold (S090, S177) and no scaffold control and target, SISVSEMEPNGTFSNNNSRNC (SEQ ID NO: 928). X-axis represents concentration of target (nM) and y-axis represents fluorescence (AU).

FIG. 20C shows a graph depicting the binding affinity between an engineered protein scaffold (S090, S177) and no scaffold control and target, TFSNNNSRNCTIENFKREFFP (SEQ ID NO: 929). X-axis represents concentration of target (nM) and y-axis represents fluorescence (AU).

FIG. 20D shows a diagram of a schematic to identify the epitope on CYSLTR2 that binds to the engineered scaffold, the N-terminal binding region was split into 3 N-terminally biotinylated peptides (Epitope 1 (SEQ ID NO: 927), Epitope 2 (SEQ ID NO: 929), Epitope 3 (SEQ ID NO: 928)) with a length of ˜21AA and an overlap of ˜10AA. Figure discloses “MERKFMSLQPSISVSEMEPNGTFSNNNSRNCTIENFKREFFP” as SEQ ID NO: 926.

FIG. 21 shows a graph depicting binding affinity improvement between an engineered protein scaffold (S005, S090, S177) and no scaffold control and target, CYSLT2R (SEQ ID NO: 928). X-axis represents concentration of target (nM) and y-axis represents fluorescence (AU).

FIG. 22A shows a graph depicting the binding affinity between an engineered protein scaffold (S177, S180, S189, S193, S194) and no scaffold control and target, CYSLTR2 Epitope (SISVSEMEPNGTFSNNNSRNC (SEQ ID NO: 928)). X-axis represents concentration of CYSLTR2 Peptide (nM) and y-axis represents fluorescence (AU).

FIG. 22B shows a graph depicting the binding affinity between an engineered protein scaffold (S177, S180, S189, S193, S194) and no scaffold control and target, CYSLTR2 Epitope (GMERKFMSLQPSISVSEMEPNGTFSNNNSRNCTIENFKREFFP (SEQ ID NO: 929)). X-axis represents concentration of CYSLTR2 Peptide (nM) and y-axis represents fluorescence (AU).

FIG. 23A shows a graph depicting the binding affinity between an engineered protein scaffold (S177, S193, S194) and no scaffold control and target, CYSLTR2 Epitope (SISVSEMEPNGTFSNNNSRNC (SEQ ID NO: 928)). X-axis represents concentration of CYSLTR2 Peptide (nM) and y-axis represents fluorescence (AU).

FIG. 23B shows a graph depicting the binding affinity between an engineered protein scaffold (S177, S193, S194) and no scaffold control and target, KCNA6 Epitope (QQQEQQPASGGGGQNGQQAMS (SEQ ID NO: 931)). X-axis represents concentration of KCNA6 Peptide (nM) and y-axis represents fluorescence (AU).

FIG. 23C shows a graph depicting the binding affinity between an engineered protein scaffold (S177, S193, S194) and no scaffold control and target, TNFRSF13B Epitope (LPPELRRQRSGEVENNSDNSGRYQ (SEQ ID NO: 932)). X-axis represents concentration of TNFRSF13B Peptide (nM) and y-axis represents fluorescence (AU).

FIG. 23D shows a graph depicting the binding affinity between an engineered protein scaffold (S177, S193, S194) and no scaffold control and target, CD74 Epitope (PPKPVSKMRMATPLLMQALPMGALP (SEQ ID NO: 933)). X-axis represents concentration of CD74 Peptide (nM) and y-axis represents fluorescence (AU).

FIG. 23E shows a graph depicting the binding affinity between an engineered protein scaffold (S177, S193, S194) and no scaffold control and target, KAAG1 Epitope (PGAAAAHLPRWPPPQLAASRREA (SEQ ID NO: 934)). X-axis represents concentration of KAAG1 Peptide (nM) and y-axis represents fluorescence (AU).

FIG. 23F shows a graph depicting the binding affinity between an engineered protein scaffold (S177, S193, S194) and no scaffold control and target, CD40LG Epitope (NKEETKKENSFEMQKGDQNPQIAAH (SEQ ID NO: 935)). X-axis represents concentration of CD40LG Peptide (nM) and y-axis represents fluorescence (AU).

FIG. 24A shows a graph depicting binding kinetics between an engineered protein scaffold (S070) and target, an 18 amino acid epitope of CLIP. Y-axis is the shift of interference pattern and is proportional to the number of CLIP Peptide molecules the engineered scaffold is binding (nm). X-axis is time(s).

FIG. 24B shows a graph depicting binding kinetics between an engineered protein scaffold (S090) and target, an 18 amino acid epitope of CLIP. Y-axis is the shift of interference pattern and is proportional to the number of CLIP Peptide molecules the engineered scaffold is binding (nm). X-axis time(s).

FIG. 25 shows a graph depicting binding kinetics between an engineered protein scaffold (S090) engineered to target an 18 amino acid epitope of CLIP and targets, an 18 amino acid epitope of CLIP and a 25 amino acid epitope of CLIP. Y-axis is the shift of interference pattern and is proportional to the number of CLIP Peptide molecules the engineered scaffold is binding (nm). X-axis is time(s).

FIG. 26 shows a graph depicting binding kinetics between an engineered protein scaffold (S005) and target, a 25 amino acid epitope of CLIP. Y-axis is the shift of interference pattern and is proportional to the number of CLIP Peptide molecules the engineered scaffold is binding (nm). X-axis is time(s).

FIG. 27A shows a graph depicting binding kinetics between an engineered protein scaffold (S098) and target, an 18 amino acid epitope of CLIP. Y-axis is the shift of interference pattern and is proportional to the number of CLIP Peptide molecules the engineered scaffold is binding (nm). X-axis is time(s).

FIG. 27B shows a graph depicting binding kinetics between an engineered protein scaffold (S098) and target, a 25 amino acid epitope of CLIP. Y-axis is the shift of interference pattern and is proportional to the number of CLIP Peptide molecules the engineered scaffold is binding (nm). X-axis is time(s).

FIG. 28 shows a graph depicting binding kinetics between an engineered protein scaffold (S058) and target, an 18 amino acid epitope of CLIP. Y-axis is the shift of interference pattern and is proportional to the number of CLIP Peptide molecules the engineered scaffold is binding (nm). X-axis is time(s).

FIG. 29A shows a graph depicting binding kinetics between an engineered protein scaffold (S068) and target, an 18 amino acid epitope of CLIP. Y-axis is the shift of interference pattern and is proportional to the number of CLIP Peptide molecules the engineered scaffold is binding (nm). X-axis is time(s).

FIG. 29B shows a graph depicting binding kinetics between an engineered protein scaffold (S068) and target, a 25 amino acid epitope of CLIP. Y-axis is the shift of interference pattern and is proportional to the number of CLIP Peptide molecules the engineered scaffold is binding (nm). X-axis is time(s).

FIG. 30A shows a graph depicting the binding affinity between an engineered protein scaffold (v1.0 is S007, v1.1 is S005, and v1.2 is S090) and no scaffold control and target, GPCR1 (CYSLTR2) (MERKFMSLQPSISVSEMEPNGTFSNNNSRNCTIENFKREFFP) (SEQ ID NO: 926)). X-axis represents concentration of the GPCR1 Peptide (nM) and y-axis represents fluorescence (AU).

FIG. 30B shows a bar graph depicting the improvement in relative binding after scaffold optimization. Each bar represents no scaffold control, and three versions of engineered scaffold proteins (v1.0 is S007, v1.1 is S005, and v1.2 is S090), respectively and y-axis represents relative binding.

FIG. 31 shows a graph depicting the binding affinity between an engineered protein scaffold (v1.0 is S007, v1.1 is S005, and v1.2 is S090) and two GPCR targets, GPCR1 (CYSLTR2) (MERKFMSLQPSISVSEMEPNGTFSNNNSRNCTIENFKREFFP (SEQ ID NO: 926)) and GPCR2 (Urotensin II) (MALTPESPSSFPGLAATGSSVPEPPGGPNATLNSSWASPTEPSSLEDLVATGTI (SEQ ID NO: 972)). X-axis represents concentration of the GPCR Peptide (nM) and y-axis represents relative fluorescent units (AU).

FIG. 32 demonstrates occlusion or inhibitor activity of an engineered scaffold protein described herein. In the present figure, Leukotriene C4's access to CysLTR2's binding site decreases after binding by an engineered scaffold protein provided herein.

FIG. 33 demonstrates top view and side view of a three-dimensional rendering of an engineered scaffold protein comprising a binding groove architecture as described herein.

FIG. 34 demonstrates cross-section views of a three-dimensional rendering of an engineered scaffold protein comprising a binding groove architecture as described herein.

FIG. 35 provides an exemplary schematic of a molecular transporter and engineered scaffold protein system, wherein the engineered scaffold protein and molecular transporter are linked via a linker.

FIG. 36 provides an exemplary schematic of a detectable moiety conjugated to an engineered scaffold protein that is bound to a target for diagnostic, sequencing, or fingerprinting applications.

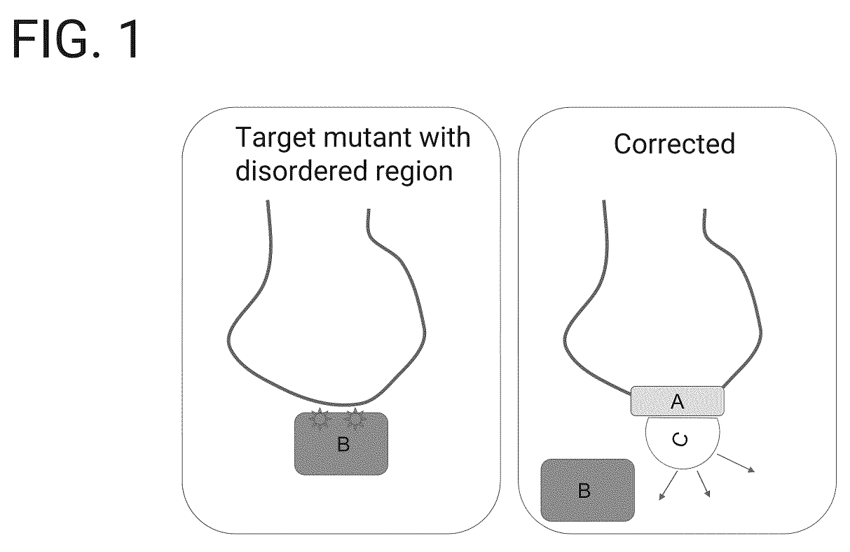

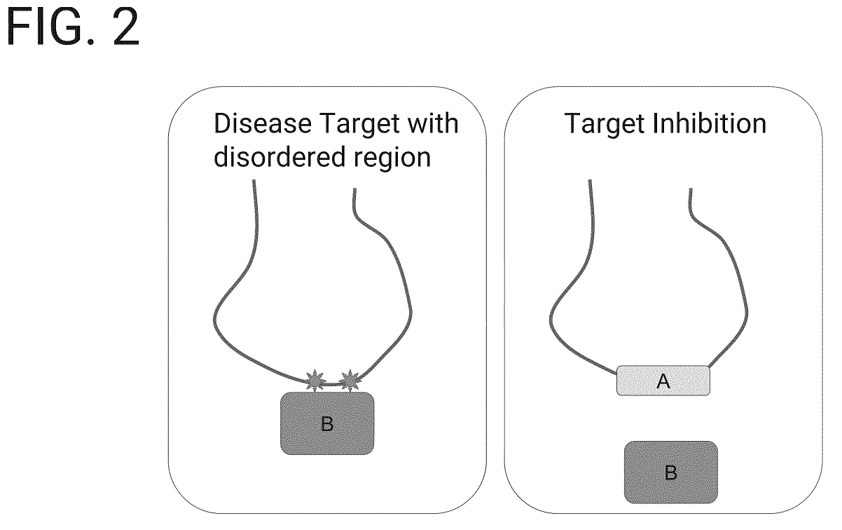

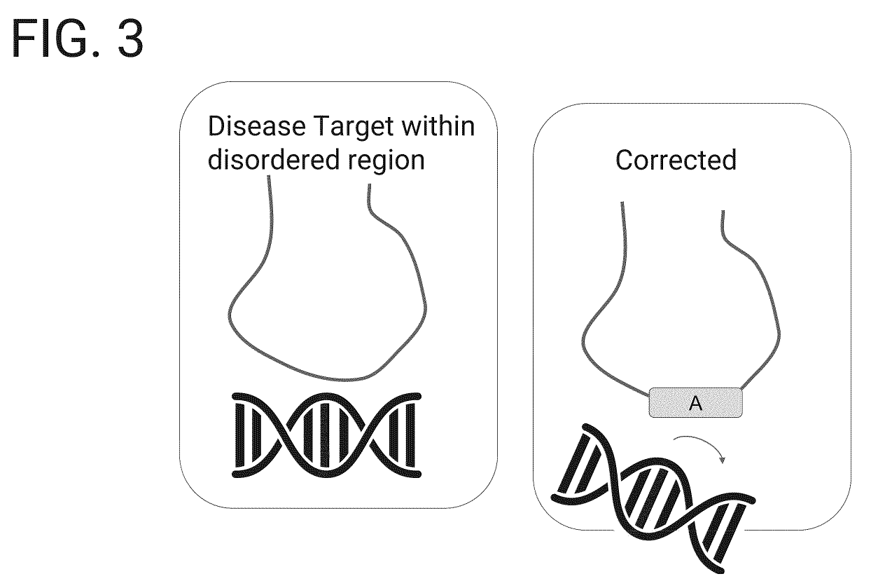

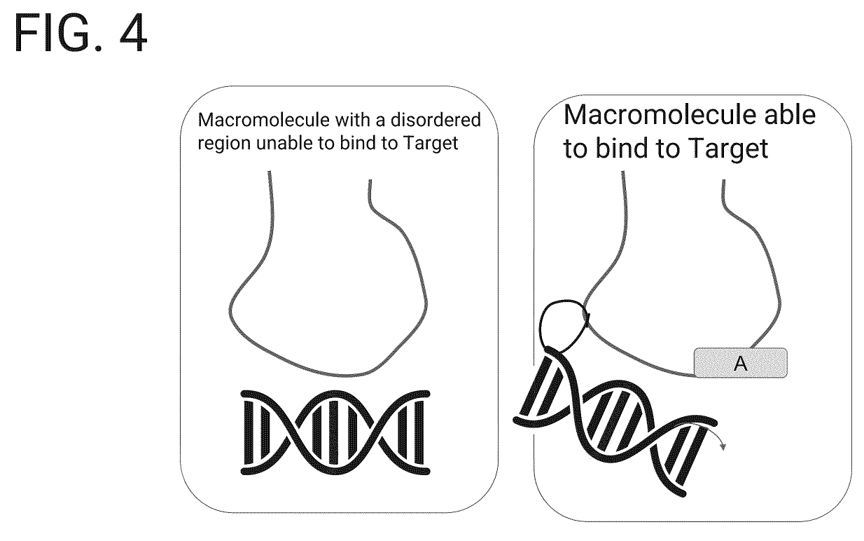

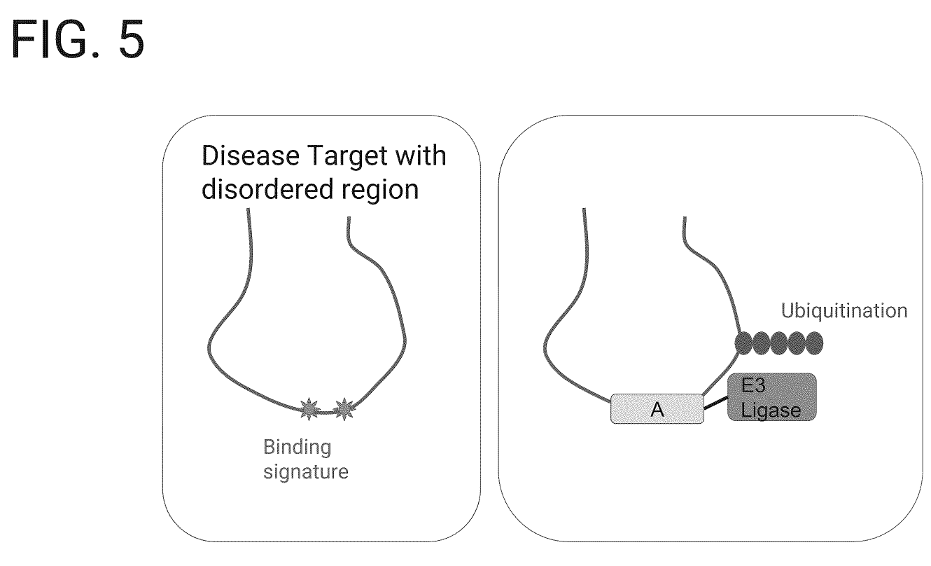

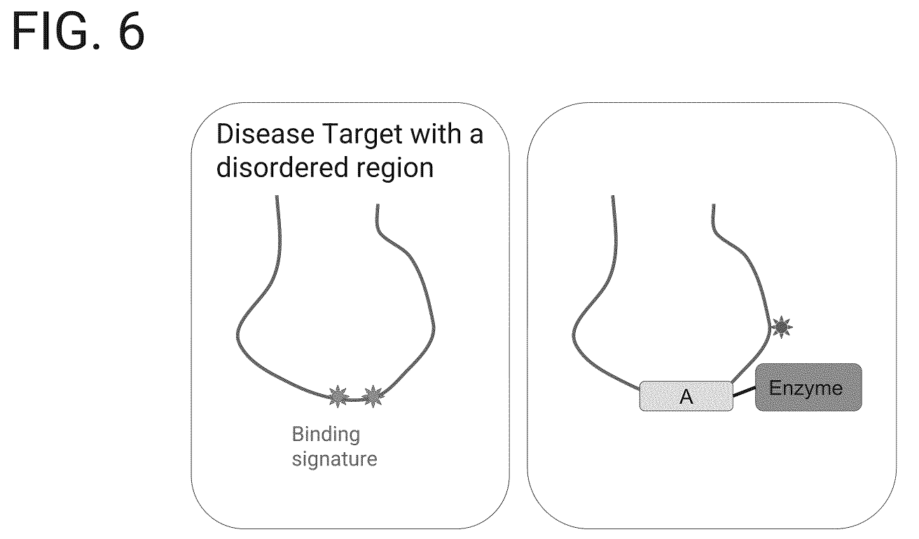

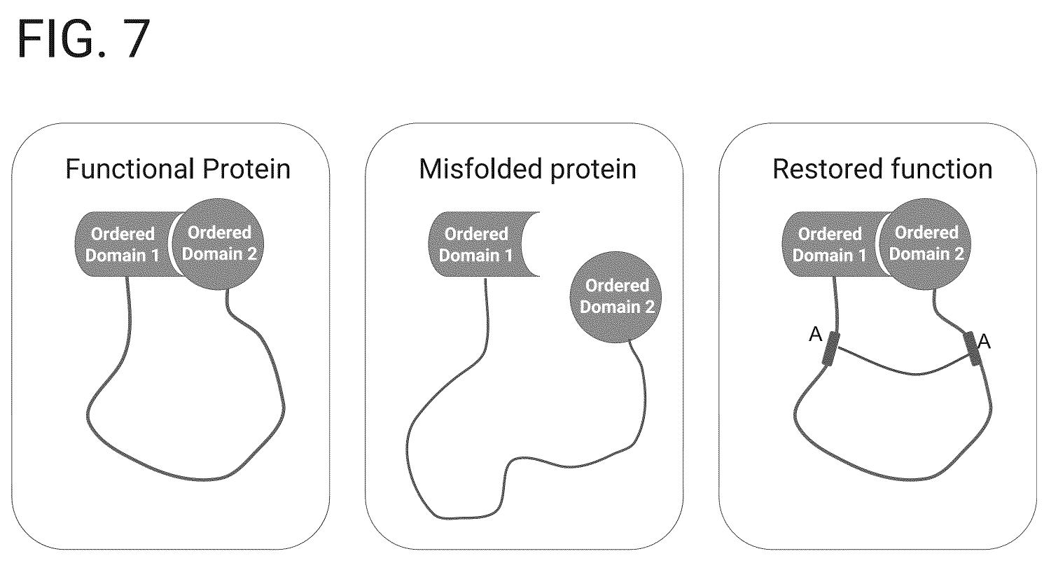

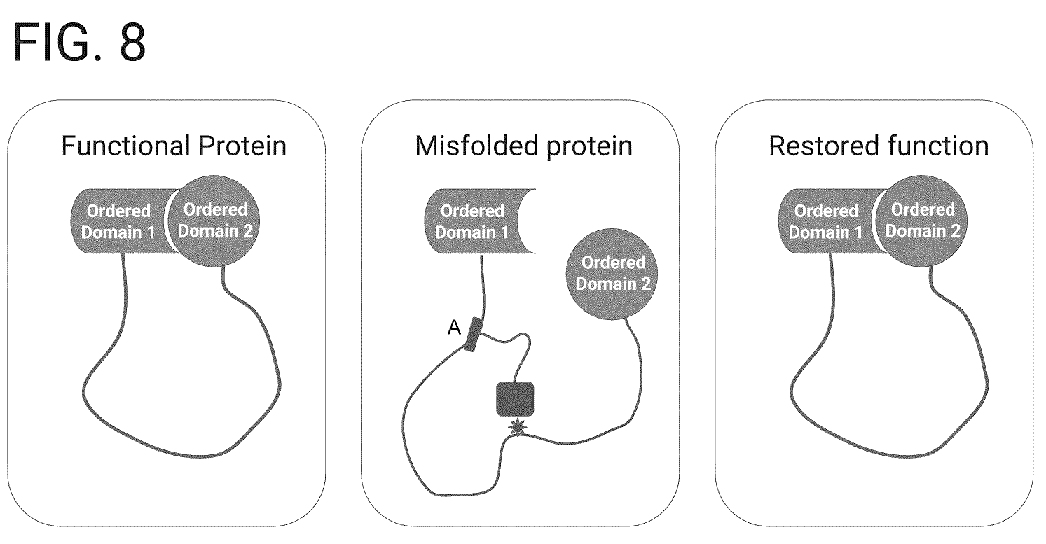

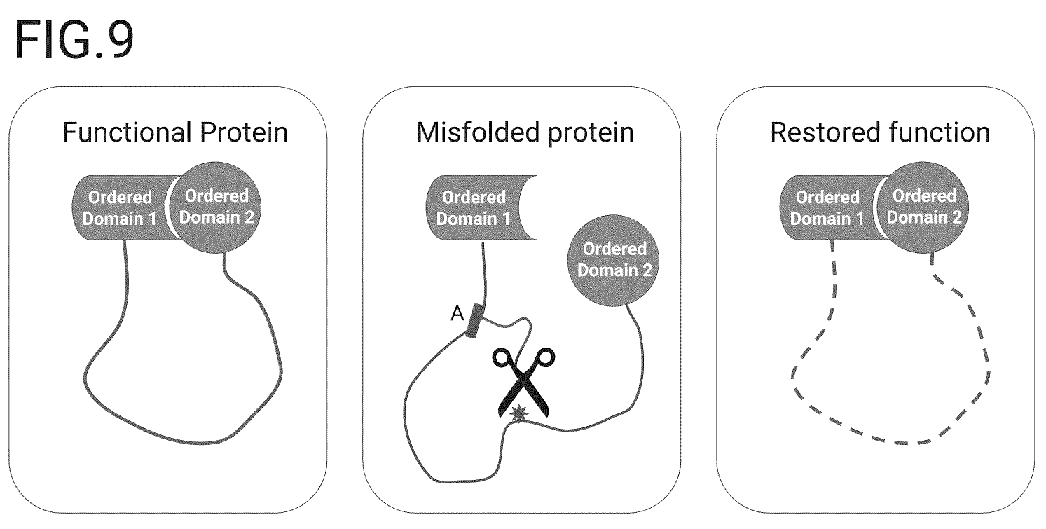

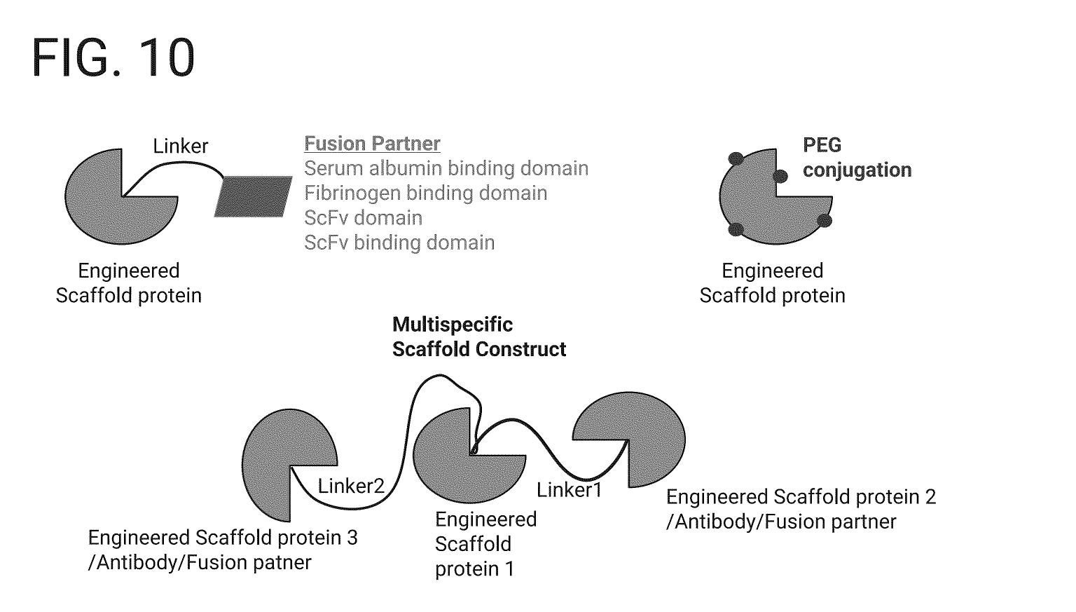

### DETAILED DESCRIPTION

Disclosed herein are compositions, systems and methods to precisely bind a known sequence of amino acids. Such compositions, systems and methods rely upon an engineered scaffold protein disclosed herein. Also disclosed herein are compositions, systems and methods to bind disordered regions of varying lengths found in target proteins. Such compositions, systems and methods bind to a protein in a sequence-specific manner based on an engineered scaffold protein. Still further disclosed herein are methods to locally edit protein structure or function in a sequence-specific manner using such an engineered scaffold protein. Yet further disclosed herein are methods to modulate protein-macromolecule interaction networks using an engineered scaffold protein disclosed herein. These engineered scaffold proteins can be used in methods to sense and detect bioanalytes, to induce local conformational changes, to specifically bind two or more targets simultaneously by fusing and linking multiple binding moieties together, and the like. to the compositions, systems and methods described herein can be used to bind macromolecular targets in the extracellular matrix and the intracellular environment. Further disclosed herein are kits, reagents, and instruments that can detect macromolecules in situ by using an engineered scaffold protein described. Such kits, reagents, and instruments can be used to detect proteins/peptides, such as those present on a bead or chip surface, for protein fingerprinting, protein sequencing, or protein detection.

As used in this application, including the appended claims, the singular forms “a,” “an,” and “the” include plural references, unless the content clearly dictates otherwise, and are used interchangeably with “at least one” and “one or more.”

The term “about,” particularly in reference to a given quantity, is meant to encompass deviations of plus or minus five percent.

The term “amino acid” as used herein refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an alpha carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. In certain instances, the use of non-natural amino acids can be utilized to enhance stability (e.g., alter the in vivo or in vitro half-life of an engineered scaffold protein) or reduce immunogenicity of an engineered scaffold protein described herein. Non-natural amino acids can also be used to facilitate certain chemical modifications (e.g., pegylation) of an engineered scaffold protein described herein. For instance, certain non-natural amino acids allow selective attachment of polymers such as PEG to an engineered scaffold protein, and thereby improving its pharmacokinetic properties.

The terms “comprises,” “comprising,” “includes,” “including,” “contains,” “containing,” and any variations thereof, as used herein, are intended to cover a non-exclusive inclusion, such that a process, method, product-by-process, or composition of matter that comprises, includes, or contains an element or list of elements does not include only those elements but can include other elements not expressly listed or inherent to such process, method, product-by-process, or composition of matter.

The terms, “bind,” “binding,” “interact” and “interacting,” as used herein, refer to an interaction between macromolecules (e.g., between two polypeptides or between a polypeptide and a nucleic acid) that can include non-covalent bonds, as well as, in some instances, convent bonds. While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Non-limiting examples of non-covalent interactions are ionic bonds, hydrogen bonds, van der Waals and hydrophobic interactions. Not all components of a binding interaction need be sequence-specific (e.g., contacts with phosphate residues in a DNA backbone), but some portions of a binding interaction may be sequence-specific. In some instances, while in a state of non-convent interaction, a covalent chemical bond between two molecules can be formed, which include, for example, cysteine-cysteine side chain bonds.

The term “binding unit,” as used herein, refers to one or more sites on a binding domain which non-covalently interacts with a target peptide so that the target peptide is associated with or bound to an engineered scaffold protein. In some instances, the portion of the target peptide which interacts with a binding unit is a disordered region. In some instances, the portion of the disordered region that interacts with a binding unit is a linear epitope. In some instances, once bound by an engineered scaffold protein, the bound portion is referred to as a “bound peptide”.

The term “binding groove architecture,” as used herein, refers to a three-dimensional configuration of a protein domain which is characterized by a concave interface in a polypeptide lobe or at least two connected polypeptide lobes with the concave interface between the two lobes, wherein binding of a target peptide induces a conformational change such that the lobe(s) fold, at least partially, together, and bury the bound portion of the target peptide in the protein.

The term “clamshell structure,” as used herein, refers to the three-dimensional architecture characteristic of a binding monomer or multimer which comprises two semi-symmetrical helices supported by a beta sheet. A clamshell structure is an example of a three-dimensional architecture characteristic having a binding groove architecture as described herein.

The term “disordered region,” as used herein, refers to a segment of a protein that assumes a linear conformation at least some of the time and/or under certain conditions. The linear segment of a protein may be a part of a larger protein that may have stable three-dimensional conformation under physiological conditions. A disordered region may also refer to segments that lack a unique 3-dimensional structure at least some of the time and/or under certain conditions. A few features associated with intrinsically disordered regions (IDRs) include, but are not limited to: higher percentage of polar or charged amino acids; known to have lower number of hydrophobic residues that prevent cooperative folding; sample a variety of 3-dimensional states that are in dynamic equilibrium under physiological conditions; and may exist in ordered states under certain physiological conditions, or in association with binding partners including proteins, nucleic acids and lipids. See, e.g., M. Madan Babu, The contribution of intrinsically disordered regions to protein function, cellular complexity, and human disease. Biochemical Society Transactions (2016). 44:1185-1200. MTOR, for instance, is a kinase that has a predicted structure consisting of alpha helices along with some regions that do not have a predicted structure (i.e., disordered regions); this is an example of a protein with an intrinsically disordered region (IDR). SDC-1 is another protein that is considered mostly “unstructured”, with a structure predicted for only a short region, and is also an example of an “intrinsically disordered protein” (IDP), whereby most or all residues do not fold into a rigid structure.

The term, “fused,” as used herein, refers to at least two sequences that are connected together, such as by a covalent bond (e.g., an amide bond or a phosphodiester bond) or by a linker. The covalent bond can be formed by a conjugation (e.g., chemical conjugation or enzymatic conjugation) reaction.

The term “fusion partner,” as used herein, refers to a molecule, an agent, a compound, a macromolecule (e.g., a protein) that is fused to an engineered scaffold protein as described herein. In some instances, the fusion partner can impart some function or activity to the fusion protein that is not provided by the engineered scaffold protein.

The term “fusion protein,” as used herein, refers to a protein comprising at least two heterologous polypeptides. In some instances, a fusion protein comprises one or more engineered scaffold proteins and fusion partners.

The term “hinge unit,” as used herein, refers to a component of an engineered scaffold protein which stabilizes the structural conformation of an engineered scaffold protein described herein and/or stabilizes the binding interaction between the target peptide and an engineered scaffold protein described herein. A hinge unit can also bind, at least partially, to a target peptide described herein.

The term “immunoglobulin unit,” as used herein, refers to an extracellular membrane-proximal peptide which is capable of recognizing antigens, supporting a binding domain, and/or to enhance structural integrity of the tertiary conformation of an engineered scaffold protein as described herein.

The term “linking unit,” as used herein, refers to a flexible peptide that connects one or more component of the binding domain provided herein and allows for the change in structural conformation of an engineered scaffold protein when a binding domain of the scaffold binds a target peptide.

The term “target peptide,” as used herein, refers to a peptide, a polypeptide, a protein domain, two or more protein domains, a peptide on the surface of a protein complex, or any combination thereof. The target peptide can be unmodified (without post-translational modifications) or modified either endogenously or exogenously.

The term “wild-type counterpart” or “WT counterpart,” as used herein, refers to a protein or portion of the protein from which an engineered protein is derived from. For example, the WT counterpart of an engineered scaffold protein described herein can be the WT protein the engineered scaffold protein is derived from prior to modification for use in accordance with the present disclosure. In another example, the WT counterpart of a binding domain described herein can be the domain or region of the WT protein the binding domain is derived from prior to modification for use in accordance with the present invention, which may be separate and/or different from the WT counterpart of an engineered scaffold protein described herein.

### Engineered Scaffold Proteins

Provided herein are compositions, systems and methods comprising an engineered scaffold protein or a use thereof. An engineered scaffold protein provided herein can bind a known sequence of amino acids. In some embodiments, the known sequence of amino acids can comprise or be a linear sequence of amino acids. A known sequence of amino acids, in some embodiments, may be or comprise an disordered region in a target peptide as described herein. Such a target peptide can have one or more disordered regions as described herein in combination with one or more ordered regions. Binding may be precise, specific, sequence-specific, or any combination thereof. Binding of a target peptide with an engineered scaffold protein described herein may induce one or more activity in vivo and/or in vitro. For example, engineered scaffold proteins and compositions comprising the same can locally edit protein structure or function in a sequence-specific manner, modulate protein-macromolecule interaction networks, sense and detect bioanalytes, induce local conformational changes, specifically bind two or more targets simultaneously by fusing and linking multiple binding moieties together, bind macromolecular targets in the extracellular matrix and the intracellular environment, sense and detect biomolecules, and the like.

In some embodiments, an engineered scaffold protein provided herein can be derived from a scaffold protein described herein. Scaffold proteins disclosed herein demonstrate binding affinity to a specific peptide sequence that can be contained in a disordered region and can be used as a starting point for designing the engineered scaffold proteins disclosed herein based on requirements of therapeutic modality, such as the size of binder, sequence to be bound, and the physiochemical properties such as solubility and temperature sensitivity. Thus, a “scaffold protein” refers to the protein used as a starting point for generation of an “engineered scaffold protein.” Such an engineered scaffold protein has modified binding properties over the starting scaffold protein, including specificity for a disordered region of a target protein. For example, scaffold proteins may be identified from any of the proteins listed in TABLE 1, based on the peptide sequence the scaffold protein is known to bind, such as those described in TABLE 3, and the disordered region of the target protein, and may be modified or engineered to provide for specific binding (e.g., high affinity binding) of the disordered region, the desired size of the engineered scaffold protein or a fusion protein thereof, or the desired specific physiochemical properties of the engineered scaffold protein or a fusion protein thereof (e.g., solubility and temperature). See, e.g., Martins, P. M., Santos, L. H., Mariano, D. et al. Propedia: a database for protein-peptide identification based on a hybrid clustering algorithm. BMC Bioinformatics 22, 1 (2021) for further examples of such proteins that have been known to complex with a peptide sequence, and protein classes that may serve as starting points for the scaffolds disclosed herein.

In some embodiments, engineered scaffold proteins provided herein comprise an amino acid sequence that is less than about 100 amino acids, about 100 amino acids to about 500 amino acids, or more than about 500 amino acids. In some embodiments, engineered scaffold proteins provided herein comprise an amino acid sequence that is at least about 100 amino acids, about 150 amino acids, about 200 amino acids, about 250 amino acids, about 300 amino acids, about 350 amino acids, about 400 amino acids, about 450 amino acids, about 500 amino acids, or greater than 500 amino acids. In some embodiments, engineered scaffold proteins provided herein comprise an amino acid sequence that is at least about 160 amino acids, about 180 amino acids, about 200 amino acids, about 220 amino acids, about 240 amino acids, about 260 amino acids, about 280 amino acids, about 300 amino acids, about 320 amino acids, about 340 amino acids, about 360 amino acids, about 380 amino acids, about 400 amino acids, about 420 amino acids, or about 440 amino acids. In certain embodiments, an engineered scaffold protein described herein is a size suitable for packaging into a vector, such as a viral vector. For example, in some embodiments, an engineered scaffold protein described herein is less than 250 amino acids.

In some embodiments, engineered scaffold proteins provided herein comprise a protein Isoelectric (pI) point of less than about 4 pI, about 4 pI to about 8 pI, or greater than 8 pI. In some embodiments, engineered scaffold proteins provided herein comprise a pI point of about 4 pI, about 4.2 pI, about 4.4 pI, about 4.6 pI, about 4.8 pI, about 5 pI, about 5.2 pI, about 5.4 pI, about 5.6 pI, about 5.8 pI, about 6 pI, about 6.2 pI, about 6.4 pI, about 6.6 pI, about 6.8 pI, about 7 pI, about 7.2 pI, about 7.4 pI, about 7.6 pI, about 7.8 pI, or about 8 pI. A person of ordinary skill in the art would understand how to determine the pI point of an engineered scaffold protein described herein.

Binding of an engineered scaffold protein or therapeutic molecule thereof may be characterized by describing its affinity for a target peptide. The affinity of an engineered scaffold protein or fusion protein thereof as described herein for its target protein, in some embodiments, is less than 5 μM, less than 1 μM, less than 500 nM, less than 200 nM, less than 100 nM, less than 50 nM, less than 20 nM, less than 10 nM, less than 1 nM, less than 100 pM, less than 10 pM, less than 1 pM, less than 100 fM, or less than 1 aM. Such affinities can be achieved by the engineered scaffold protein or fusion protein thereof being specific for a particular disordered region of a target protein.

In some embodiments, an engineered scaffold protein provided herein comprises one or more amino acid alterations relative to a WT counterpart. In some embodiments, the one or more amino acid alterations comprises 1 amino acid alteration, 2 amino acid alterations, 3 amino acid alterations, 4 amino acid alterations, 5 amino acid alterations, 6 amino acid alterations, 7 amino acid alterations, 8 amino acid alterations, 9 amino acid alterations, 10 amino acid alterations, 11 amino acid alterations, 12 amino acid alterations, 13 amino acid alterations, 14 amino acid alterations, 15 amino acid alterations, 16 amino acid alterations, 17 amino acid alterations, 18 amino acid alterations, 19 amino acid alterations, 20 amino acid alterations, or more, relative to a WT counterpart.

In some embodiments, the one or more amino acid alterations is one or more amino acid substitutions, deletions, insertions, or any combination thereof. In some embodiments, the one or more amino acid substitution is one or more conservative substitution, one or more non-conservative substitution, or combinations thereof. In some embodiments, the one or more amino acid alterations are one or more conservative substitutions. A conservative substitution as described herein, refers to the replacement of one amino acid for another such that the replacement takes place within a family of amino acids that are related in their side chains. Conversely, the term “non-conservative substitution” as used herein refers to the replacement of one amino acid residue for another that does not have a related side chain. Substitutions can be for naturally occurring or synthetic amino acids, as well as amino acid analogs and amino acid mimetics. Genetically encoded amino acids can be divided into four families having related side chains: (1) acidic (negatively charged): Asp (D), Glu (E); (2) basic (positively charged): Lys (K), Arg (R), His (H); (3) non-polar (hydrophobic): Cys (C), Ala (A), Val (V), Leu (L), Ile (I), Pro (P), Phe (F), Met (M), Trp (W), Gly (G), Tyr (Y), with non-polar also being subdivided into: (i) strongly hydrophobic: Ala (A), Val (V), Leu (L), Ile (I), Met (M), Phe (F); and (ii) moderately hydrophobic: Gly (G), Pro (P), Cys (C), Tyr (Y), Trp (W); and (4) uncharged polar: Asn (N), Gln (Q), Ser(S), Thr (T). Amino acids may be related by aliphatic side chains: Gly (G), Ala (A), Val (V), Leu (L), Ile (I), Ser(S), Thr (T), with Ser(S) and Thr (T) optionally being grouped separately as aliphatic-hydroxyl; Amino acids may be related by aromatic side chains: Phe (F), Tyr (Y), Trp (W). Amino acids may be related by amide side chains: Asn (N), Gln (Q). Amino acids may be related by sulfur-containing side chains: Cys (C) and Met (M). In some embodiments, the one or more amino acid alterations are one or more non-conservative substitutions. In some embodiments, the one or more amino acid alterations are substitutions of one or more hydrophobic residues with one or more charged residues or polar residues, or combinations thereof. For example, one or more of a Ala (A), Val (V), Leu (L), Ile (I), Pro (P), Phe (F), Met (M), Trp (W), Gly (G), or Tyr (Y) may be substituted with one or more of Asp (D), Glu (E), Lys (K), Arg (R), His (H), Asn (N), Gln (Q), Ser(S), Thr (T), or any combination thereof.

In some embodiments, one or more amino acid alterations in an engineered scaffold protein provided herein can result in an increase binding activity comprising binding more selectively, binding more frequently, more rapidly, with greater duration, with greater affinity, or with some combination of the foregoing relative to a corresponding WT. In some embodiments, one or more amino acid alterations in an engineered scaffold protein provided herein can result in a decrease of immunogenicity when administered to a subject relative to a corresponding WT. In some embodiments, one or more amino acid alterations in an engineered scaffold protein provided herein can result in an increase of solubility relative to a corresponding WT. In some embodiments, one or more amino acid alterations in an engineered scaffold protein provided herein can result in an increase of stability relative to a corresponding WT. In some embodiments, one or more amino acid alterations in an engineered scaffold protein provided herein can result in modification of hydrophobicity relative to a corresponding WT. Modification of hydrophobicity relative to a corresponding WT can be an increase in hydrophobicity or a decrease in hydrophobicity relative to a corresponding WT depending on the desired activity of the engineered scaffold protein. In some embodiments, one or more amino acid alterations in an engineered scaffold protein provided herein can result in modification of hydrophilicity relative to a corresponding WT. Modification of hydrophilicity relative to a corresponding WT can be an increase in hydrophilicity or a decrease in hydrophilicity relative to a corresponding WT depending on the desired activity of the engineered scaffold protein. In some embodiments, one or more amino acid alterations in an engineered scaffold protein provided herein can result in a modification of surface charge relative to a corresponding WT. Modification of surface charge relative to a corresponding WT can be an increase in surface charge or a decrease in surface charge relative to a corresponding WT depending on the desired activity of the engineered scaffold protein. Desired activity can be related to binding activity, solubility, and the like as described herein.

In some embodiments, an engineered scaffold protein provided herein comprises an amino acid sequence that is at least about 60%, about 65%, about 70%, about 75%, about 80%, about 85%, about 90%, about 95%, or 100% identical to any one of the amino acid sequences set forth in TABLE 6. In some embodiments, an engineered scaffold protein provided herein comprises an amino acid sequence that is at least about 60% identical to any one of the amino acid sequences set forth in TABLE 6. In some embodiments, an engineered scaffold protein provided herein comprises an amino acid sequence that is at least about 65% identical to any one of the amino acid sequences set forth in TABLE 6. In some embodiments, an engineered scaffold protein provided herein comprises an amino acid sequence that is at least about 70% identical to any one of the amino acid sequences set forth in TABLE 6. In some embodiments, an engineered scaffold protein provided herein comprises an amino acid sequence that is at least about 75% identical to any one of the amino acid sequences set forth in TABLE 6. In some embodiments, an engineered scaffold protein provided herein comprises an amino acid sequence that is at least about 80% identical to any one of the amino acid sequences set forth in TABLE 6. In some embodiments, an engineered scaffold protein provided herein comprises an amino acid sequence that is at least about 85% identical to any one of the amino acid sequences set forth in TABLE 6. In some embodiments, an engineered scaffold protein provided herein comprises an amino acid sequence that is at least about 90% identical to any one of the amino acid sequences set forth in TABLE 6. In some embodiments, an engineered scaffold protein provided herein comprises an amino acid sequence that is at least about 95% identical to any one of the amino acid sequences set forth in TABLE 6. In some embodiments, an engineered scaffold protein provided herein comprises an amino acid sequence that is 100% identical to any one of the amino acid sequences set forth in TABLE 6.

In some embodiments, an engineered scaffold protein provided herein comprises one or more amino acid alterations relative to any one of the amino acid sequences set forth in TABLE 6. In some embodiments, the one or more amino acid alterations comprises 1 amino acid alteration, 2 amino acid alterations, 3 amino acid alterations, 4 amino acid alterations, 5 amino acid alterations, 6 amino acid alterations, 7 amino acid alterations, 8 amino acid alterations, 9 amino acid alterations, 10 amino acid alterations, 11 amino acid alterations, 12 amino acid alterations, 13 amino acid alterations, 14 amino acid alterations, 15 amino acid alterations, 16 amino acid alterations, 17 amino acid alterations, 18 amino acid alterations, 19 amino acid alterations, 20 amino acid alterations, or more relative to any one of the amino acid sequences set forth in TABLE 6. In some embodiments, an engineered scaffold protein comprising one or more amino acid alterations is a variant of an engineered scaffold protein described herein. It is understood that reference to an engineered scaffold protein also refers to an engineered scaffold protein variant as described herein.

In some embodiments, one or more amino acid alterations in an engineered scaffold protein provided herein can result in an increase binding activity comprising binding more selectively, binding more frequently, more rapidly, with greater duration, with greater affinity, or some combination of the foregoing relative to any one of the amino acid sequences set forth in TABLE 1. In some embodiments, one or more amino acid alterations in an engineered scaffold protein provided herein can result in a decrease of immunogenicity when administered to a subject relative to any one of the amino acid sequences set forth in TABLE 1. In some embodiments, one or more amino acid alterations in an engineered scaffold protein provided herein can result in an increase of solubility relative to any one of the amino acid sequences set forth in TABLE 1. In some embodiments, one or more amino acid alterations in an engineered scaffold protein provided herein can result in an increase of stability relative to any one of the amino acid sequences set forth in TABLE 1. In some embodiments, one or more amino acid alterations in an engineered scaffold protein provided herein can result in modification of hydrophobicity relative to any one of the amino acid sequences set forth in TABLE 1. Modification of hydrophobicity can be an increase in hydrophobicity or a decrease in hydrophobicity relative to any one of the amino acid sequences set forth in TABLE 1 depending on the desired activity of the engineered scaffold protein. In some embodiments, one or more amino acid alterations in an engineered scaffold protein provided herein can result in modification of hydrophilicity relative to any one of the amino acid sequences set forth in TABLE 1. Modification of hydrophilicity can be an increase in hydrophilicity or a decrease in hydrophilicity relative to any one of the amino acid sequences set forth in TABLE 1 depending on the desired activity of the engineered scaffold protein. In some embodiments, one or more amino acid alterations in an engineered scaffold protein provided herein can result in a modification of surface charge relative to any one of the amino acid sequences set forth in TABLE 1. Modification of surface charge can be an increase in surface charge or a decrease in surface charge relative to any one of the amino acid sequences set forth in TABLE 1 depending on the desired activity of the engineered scaffold protein.

In some embodiments, compositions, systems and methods described herein comprise the use of one or more engineered scaffold protein. In such embodiments, the engineered scaffold protein may interact or bind with one target peptide or more than one target peptide. In some embodiments, compositions, systems and methods described herein comprise the use of two, three, or four or more engineered scaffold protein, wherein the engineered scaffold proteins target one, two, three, four, or more target peptides.

In some embodiments, engineered scaffold proteins provided herein comprise one or more of a: binding domain, immunoglobulin unit, linker, fusion partner, epitope masking unit, a molecular transporter, or any combination thereof. Each component, including molecular transporters, are further described herein.

In some embodiments, engineered scaffold proteins provided herein are also engineered to reduce immunogenicity of said engineered scaffold protein. For example, in some embodiments, a scaffold protein as described herein is selected by determining prevalent genes, such as prevalent HLA alleles, as the scaffold protein from which engineered scaffold proteins are derived from. In another example, where an engineered scaffold protein comprises one or more amino acid alterations, in some embodiments, such alterations are of amino acids that can be buried or distal from the surface of the binding domain, so that the binding unit can be potent and selective to a target peptide described herein. In yet another example, an engineered scaffold protein can comprise an epitope masking unit or a use thereof to hide the one or more amino acid alterations. In a further example, an engineered scaffold protein can exclude one or more immunoglobulin units which can function to deter direct binding and trigger an immune response.

### Binding Domain

Provided herein are compositions, systems and methods comprising an engineered scaffold proteins or a use thereof, wherein the engineered scaffold protein comprises one or more binding domains. In some embodiments, a binding domain described herein can be engineered to bind to a target peptide as described herein. In some embodiments, a binding domain described herein can be engineered to bind to a disordered region in a target peptide as described herein.

In some embodiments, an engineered scaffold protein described herein comprises 1, 2, 3, 4 or more binding domains. In some embodiments, where an engineered scaffold protein comprises 2 or more binding domains, such binding domains may be covalently attached, attached by one or more linkers, or combinations thereof. Examples of organization of binding domains in constructs described herein are set forth in Example 4.

In some embodiments, a binding domain comprises one or more: binding units, hinge units, linking units, linkers, or combinations thereof.

### Binding Unit

In some embodiments, an engineered scaffold protein provided herein comprises a binding domain comprising one or more binding units. In some embodiments, a binding unit non-covalently binds to a target peptide. In some embodiments, a binding unit non-covalently binds to one or more target peptides. In some embodiments, a binding unit binds to a disordered region in a target peptide. In some embodiments, a binding unit binds to a target peptide in a sequence-specific manner.

In some embodiments, a binding unit provided herein comprises one or more amino acids that are capable of binding to one or more amino acids of a target peptide. In some embodiments, a binding unit described herein comprises one or more amino acids that are capable of binding to one or more amino acids of a disordered region in a target peptide. In some embodiments, a binding unit described herein comprises one or more amino acids that are capable of binding to one or more amino acids of a disordered region in a target peptide in a sequence-specific manner. It is understood that not all of the one or more amino acid residues that are capable of binding to a target peptide need to bound by the one or more amino acids of the disordered region of the target peptide for sequence-specificity to occur. In some embodiments, the one or more amino acid residues that are capable of binding to a target peptide may not be contiguous in the primary structure of an engineered scaffold protein described herein, but rather become comprised in the binding unit of the engineered scaffold protein once the engineered scaffold protein is in its tertiary structure. In some embodiments, the one or more amino acids of the binding unit that are capable of binding to a target peptide are oriented in the binding unit to optimize binding ability to the disordered region in the target peptide. In some embodiments, the one or more amino acids of the binding unit that are capable of binding to a target peptide are antisense to one or more amino acids of a disordered region in a target peptide as determined by corresponding sense-antisense amino acid pairing. Such amino acid pairing is described in Stambuk et al., Theory in Biosciences, 123 (4): 265-275 (2005). In some embodiments, the one or more amino acids of the binding unit that are capable of binding to a target peptide form at least one non-covalent interaction with one or more amino acids of a disordered region in a target peptide as determined by corresponding sense-antisense amino acid pairing.

In some embodiments, upon folding into its tertiary structure, the binding unit can comprise a three-dimensional conformation comprising one or more amino acids which are antisense to one or more amino acids of a disordered region in a target peptide as determined by corresponding sense-antisense amino acid pairing. In some embodiments, upon folding into its tertiary structure, the binding unit can comprise a three-dimensional conformation comprising one or more amino acids that are capable of forming at least one non-covalent interaction with one or more amino acids of a disordered region in a target peptide as determined by corresponding sense-antisense amino acid pairing.

In some embodiments, binding unit provided herein comprises an elongated conformation. In some embodiments, a binding unit provided herein comprises one or more secondary structural motifs. In some embodiments, a binding unit comprises one or more helices. Suitable helices include alpha helices, 3.10 helices and pi helices. In some embodiments, a binding unit provided herein comprises one or more helices. In some embodiments, a binding unit provided herein comprises one or more, two or more, three or more, four to more, or five or more helices. In some embodiments, a binding domain described herein comprises two binding units wherein each binding unit is a helix. In some embodiments, a binding domain described herein comprises two helices. In some embodiments, a binding domain described herein comprises two helices, wherein the two helices are semi-symmetrical. In some embodiments where a binding domain comprises two helices, the helices run anti-parallel in the three-dimensional conformation of the binding domain.

In some embodiments, a binding unit provided herein comprises one or more alpha helices. In some embodiments, a binding unit provided herein comprises one or more, two or more, three or more, four to more, or five or more alpha helices. In some embodiments, a binding domain described herein comprises two binding units wherein each binding unit is an alpha helix. In some embodiments, a binding domain described herein comprises two alpha helices. In some embodiments where a binding domain comprises two alpha helices, the alpha helices run anti-parallel in the three-dimensional conformation of the binding domain.

In some embodiments, a binding unit comprises an amino acid sequence which is less than 40 amino acids, between 40 and 200 amino acids, or greater than 200 amino acids. In some embodiments, a binding unit provided herein comprises an amino acid sequence that is about 40 amino acids, 60 amino acids, 80 amino acids, 100 amino acids, 120 amino acids, 140 amino acids, 160 amino acids, 180 amino acids, or 200 amino acids in length. In some embodiments, a binding unit provided herein comprises an amino acid sequence that is about 70 amino acids, 75 amino acids, 80 amino acids, 85 amino acids, 90 amino acids, 95 amino acids, 100 amino acids, 105 amino acids, or about 110 amino acids.

In some embodiments, a binding unit provided herein comprises one or more amino acid alterations relative to a WT counterpart. In some embodiments, a binding unit comprising one or more amino acid alterations is a variant of a binding unit described herein. It is understood that reference to a binding unit also refers to a binding unit variant as described herein. In some embodiments, the one or more amino acid alterations comprises 1 amino acid alteration, 2 amino acid alterations, 3 amino acid alterations, 4 amino acid alterations, 5 amino acid alterations, 6 amino acid alterations, 7 amino acid alterations, 8 amino acid alterations, 9 amino acid alterations, 10 amino acid alterations, 11 amino acid alterations, 12 amino acid alterations, 13 amino acid alterations, 14 amino acid alterations, 15 amino acid alterations, 16 amino acid alterations, 17 amino acid alterations, 18 amino acid alterations, 19 amino acid alterations, 20 amino acid alterations, or more relative to a WT counterpart. In some embodiments, one or more amino acid alterations in a binding unit provided herein can result in an increase binding activity comprising binding more selectively, binding more frequently, more rapidly, with greater duration, with greater affinity, or with some combination of the foregoing relative to a WT counterpart.

In some embodiments, a binding unit comprises an amino acid sequence that is at least about 60%, about 65%, about 70%, about 75%, about 80%, about 85%, about 90%, about 95%, or about 100% identical to any one of the sequences set forth in TABLE 4. In some embodiments, a binding unit provided herein comprises an amino acid sequence that is at least about 60% identical to any one of the amino acid sequences set forth in TABLE 4. In some embodiments, a binding unit provided herein comprises an amino acid sequence that is at least about 65% identical to any one of the amino acid sequences set forth in TABLE 4. In some embodiments, a binding unit provided herein comprises an amino acid sequence that is at least about 70% identical to any one of the amino acid sequences set forth in TABLE 4. In some embodiments, a binding unit provided herein comprises an amino acid sequence that is at least about 75% identical to any one of the amino acid sequences set forth in TABLE 4. In some embodiments, a binding unit provided herein comprises an amino acid sequence that is at least about 80% identical to any one of the amino acid sequences set forth in TABLE 4. In some embodiments, a binding unit provided herein comprises an amino acid sequence that is at least about 85% identical to any one of the amino acid sequences set forth in TABLE 4. In some embodiments, a binding unit provided herein comprises an amino acid sequence that is at least about 90% identical to any one of the amino acid sequences set forth in TABLE 4. In some embodiments, a binding unit provided herein comprises an amino acid sequence that is at least about 95% identical to any one of the amino acid sequences set forth in TABLE 4. In some embodiments, a binding unit provided herein comprises an amino acid sequence that is 100% identical to any one of the amino acid sequences set forth in TABLE 4.

In some embodiments, a binding unit provided herein comprises one or more amino acid alterations relative to any one of the amino acid sequences set forth in TABLE 4. In some embodiments, the one or more amino acid alterations comprises 1 amino acid alteration, 2 amino acid alterations, 3 amino acid alterations, 4 amino acid alterations, 5 amino acid alterations, 6 amino acid alterations, 7 amino acid alterations, 8 amino acid alterations, 9 amino acid alterations, 10 amino acid alterations, 11 amino acid alterations, 12 amino acid alterations, 13 amino acid alterations, 14 amino acid alterations, 15 amino acid alterations, 16 amino acid alterations, 17 amino acid alterations, 18 amino acid alterations, 19 amino acid alterations, 20 amino acid alterations, or more relative to any one of the amino acid sequences set forth in TABLE 4. In some embodiments, one or more amino acid alterations in a binding unit provided herein can result in an increase binding activity comprising binding more selectively, binding more frequently, more rapidly, with greater duration, with greater affinity, or with some combination of the foregoing relative to to any one of the amino acid sequences set forth in TABLE 4.

### Hinge Unit

In some embodiments, an engineered scaffold protein provided herein comprises a binding domain comprising one or more hinge units. In some embodiments, the hinge unit stabilizes the structural conformation of an open-unbound engineered scaffold protein, open-bound engineered scaffold protein, closed-unbound engineered scaffold protein, closed-bound engineered scaffold protein, partially bound engineered scaffold protein, or any combination thereof. A hinge unit can also bind to one or more target peptides. In some embodiments, a hinge unit can also be engineered to non-covalently bind to one or more target peptides. In some embodiments, a hinge unit can bind to a disordered region in a target peptide. In some embodiments, a hinge unit can bind to a target peptide in a sequence-specific manner. In some embodiments, a hinge unit and a binding unit can work together cooperatively, collaboratively, and/or synergistically to bind to a target peptide.

In some embodiments, a binding domain provided herein comprises one or more hinge units. In some embodiments, a binding domain provided herein comprises 1, 2, 3, 4, 5, or more hinge units as described herein. In some embodiments, a hinge unit comprises a linear, or a partially linear, three-dimensional conformation. In some embodiments, a hinge unit comprises a concave, or a partially concave, three-dimensional conformation. In some embodiments, a hinge unit comprises a convex, or a partially convex, three-dimensional conformation. In some embodiments, a hinge unit comprises a flat, or partially flat, three-dimensional conformation. In some embodiments, a hinge unit described herein comprises a complementary, or partially complementary, conformation to a disordered region in a target peptide as determined by corresponding sense-antisense amino acid pairing. In some embodiments, a hinge unit described herein comprises a flexible, or partially flexible, configuration to conform to the three-dimensional structure of the disordered region in a target peptide described herein. In some embodiments, the hinge unit comprises non-covalent surface forces or charges that can drive binding of the target peptide to the binding domain. Non-covalent surface forces or charges include hydrophobicity, hydrophilicity, and polarity of the surface of the hinge unit.

In some embodiments, a hinge unit comprises one or more linear peptide strands, secondary structural motifs (e.g., beta sheets), one or more covalent interactions (e.g., disulfide bonds), one or more non-covalent interactions (e.g., hydrogen bonds, salt bridges, etc.), chemical agents (e.g., DMSO, PEG, starch, etc.), or combinations thereof.

In some embodiments, one or more hinge unit is connected to the one or more binding unit. In some embodiments, a binding domain comprises two binding units and one hinge unit, wherein the two binding units are connected by the hinge unit.

In some embodiments, a hinge unit provided herein comprises one or more secondary structural motifs. In some embodiments, a hinge unit provided herein comprises one or more β-sheet proteins. In some embodiments, the one or more β-sheet proteins form one or more β-sheets. In some embodiments, the one or more β-sheets each comprise about 2 to about 20 or more strands of β-sheet proteins. In some embodiments, the one or more β-sheets each comprise about 4 strands, 5 strands, 6 strands, 7 strands, 8 strands, 9 strands, 10 strands, 11 strands, or about 12 strands of β-sheet proteins. In some embodiments, the one or more β-sheets each comprise about 6 to about 10 strands of β-sheet proteins. In some embodiments, a hinge unit provided herein comprises one or more, two or more, three or more, four to more, or five or more β-sheets.

In some embodiments, a hinge unit comprises an amino acid sequence which is less than 40 amino acids, between 40 and 200 amino acids, or greater than 200 amino acids. In some embodiments, a hinge unit provided herein comprises an amino acid sequence that is about 40 amino acids, 60 amino acids, 80 amino acids, 100 amino acids, 120 amino acids, 140 amino acids, 160 amino acids, 180 amino acids, or 200 amino acids in length. In some embodiments, a hinge unit provided herein comprises an amino acid sequence that is about 70 amino acids, 75 amino acids, 80 amino acids, 85 amino acids, 90 amino acids, 95 amino acids, 100 amino acids, 105 amino acids, or about 110 amino acids.

In some embodiments, a hinge unit provided herein comprises one or more amino acid alterations relative to a WT counterpart. In some embodiments, a hinge unit comprising one or more amino acid alterations is a variant of a hinge unit described herein. It is understood that reference to a hinge unit also refers to a hinge unit variant as described herein. In some embodiments, the one or more amino acid alterations comprises 1 amino acid alteration, 2 amino acid alterations, 3 amino acid alterations, 4 amino acid alterations, 5 amino acid alterations, 6 amino acid alterations, 7 amino acid alterations, 8 amino acid alterations, 9 amino acid alterations, 10 amino acid alterations, 11 amino acid alterations, 12 amino acid alterations, 13 amino acid alterations, 14 amino acid alterations, 15 amino acid alterations, 16 amino acid alterations, 17 amino acid alterations, 18 amino acid alterations, 19 amino acid alterations, 20 amino acid alterations, or more relative to a WT counterpart. In some embodiments, one or more amino acid alterations in a hinge unit provided herein can result in an increase binding activity comprising binding more selectively, binding more frequently, more rapidly, with greater duration, with greater affinity, or with some combination of the foregoing relative to a WT counterpart.

In some embodiments, a hinge unit comprises an amino acid sequence that is at least about 60%, about 65%, about 70%, about 75%, about 80%, about 85%, about 90%, about 95%, or about 100% identical to any one of the sequences set forth in TABLE 5. In some embodiments, a hinge unit provided herein comprises an amino acid sequence that is at least about 60% identical to any one of the amino acid sequences set forth in TABLE 5. In some embodiments, a hinge unit provided herein comprises an amino acid sequence that is at least about 65% identical to any one of the amino acid sequences set forth in TABLE 5. In some embodiments, a hinge unit provided herein comprises an amino acid sequence that is at least about 70% identical to any one of the amino acid sequences set forth in TABLE 5. In some embodiments, a hinge unit provided herein comprises an amino acid sequence that is at least about 75% identical to any one of the amino acid sequences set forth in TABLE 5. In some embodiments, a hinge unit provided herein comprises an amino acid sequence that is at least about 80% identical to any one of the amino acid sequences set forth in TABLE 5. In some embodiments, a hinge unit provided herein comprises an amino acid sequence that is at least about 85% identical to any one of the amino acid sequences set forth in TABLE 5. In some embodiments, a hinge unit provided herein comprises an amino acid sequence that is at least about 90% identical to any one of the amino acid sequences set forth in TABLE 5. In some embodiments, a hinge unit provided herein comprises an amino acid sequence that is at least about 95% identical to any one of the amino acid sequences set forth in TABLE 5. In some embodiments, a hinge unit provided herein comprises an amino acid sequence that is 100% identical to any one of the amino acid sequences set forth in TABLE 5.

In some embodiments, a hinge unit provided herein comprises one or more amino acid alterations relative to any one of the amino acid sequences set forth in TABLE 5. In some embodiments, the one or more amino acid alterations comprises 1 amino acid alteration, 2 amino acid alterations, 3 amino acid alterations, 4 amino acid alterations, 5 amino acid alterations, 6 amino acid alterations, 7 amino acid alterations, 8 amino acid alterations, 9 amino acid alterations, 10 amino acid alterations, 11 amino acid alterations, 12 amino acid alterations, 13 amino acid alterations, 14 amino acid alterations, 15 amino acid alterations, 16 amino acid alterations, 17 amino acid alterations, 18 amino acid alterations, 19 amino acid alterations, 20 amino acid alterations, or more relative to any one of the amino acid sequences set forth in TABLE 5. In some embodiments, one or more amino acid alterations in a hinge unit provided herein can result in an increase binding activity comprising binding more selectively, binding more frequently, more rapidly, with greater duration, with greater affinity, or with some combination of the foregoing relative to to any one of the amino acid sequences set forth in TABLE 5.

In some embodiments, the one or more binding units and one or more hinge units may be covalently connected, connected by a linking unit, or linked by a linker as described herein. In some embodiments, the one or more binding units and one or more hinge units is a monomer. In some embodiments, the hinge unit is connected or attached to the N terminus of the binding unit. In some embodiments, the one or more binding units and one or more hinge units are connected as a monomer. In some embodiments, the one or more binding units and one or more hinge units are multimeric. In some embodiments, the one or more binding units and one or more hinge units are multimeric wherein the one or more binding units and one or more hinge units form as a binding domain in the presence of a target peptide.

### Linking Units

In some embodiments, an engineered scaffold protein provided herein comprises a binding domain comprising one or more linking units. some embodiments, a linking unit comprises one or more of a beta-sheet strand, an alpha helix, a linear peptide, or combinations thereof. In some embodiments, a linking unit described herein connects a set of binding domain components. For example, one or more linking unit connects a set each of binding domain components comprising a binding unit and a hinge unit where the one or more linking unit is located in between the set. In some embodiments, an engineered scaffold provided herein comprises 1, 2, 3, 4, 5, or more linking units. Linking units can also be referred to as linkers as described in detail herein.

### Immunoglobulin Unit

In some embodiments, an engineered scaffold protein provided herein comprises a binding domain comprising one or more immunoglobulin units. In some embodiments, an engineered scaffold comprising an immunoglobulin unit described herein may be useful in compositions or methods for inducing an immune response. In some embodiments, an engineered scaffold provided herein comprises one or two immunoglobulin units. An immunoglobulin unit described herein can be connected to one or more of a binding unit and/or a hinge unit covalently, by one or more linking unit, or by one or more linker as described herein.

In some embodiments, the immunogenicity of an engineered scaffold protein described herein is improved by removing the one or more immunoglobulin units, thereby reducing the risk of an autoimmune response.

### Binding Groove Architecture

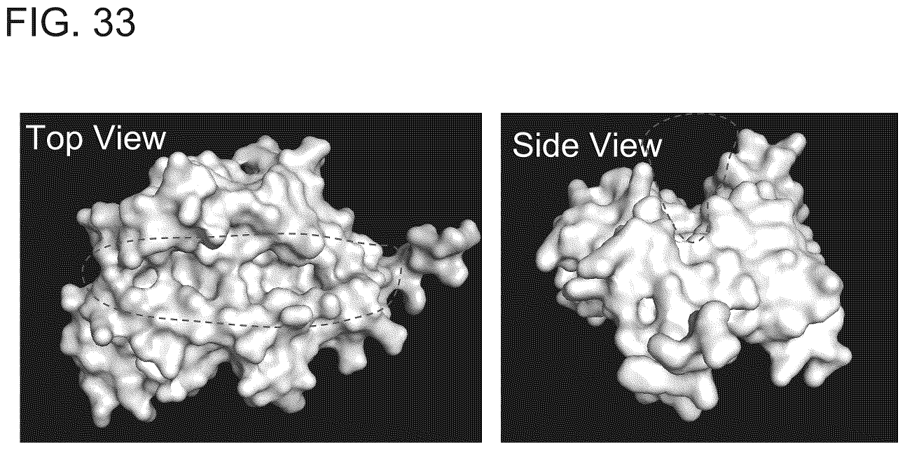

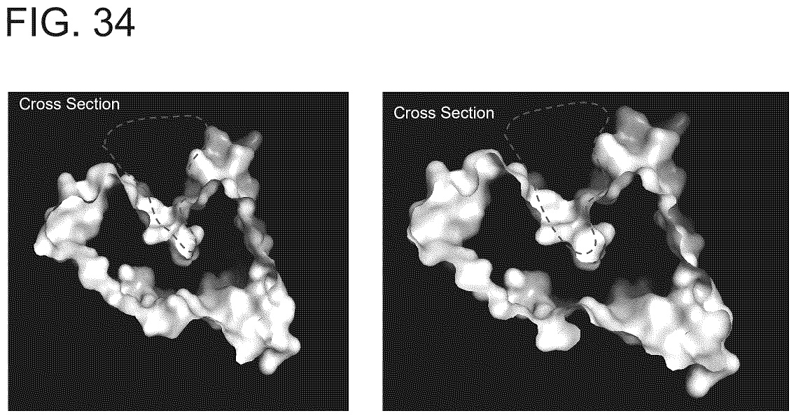

In some embodiments, a binding domain comprises a three-dimensional structure referred to herein as a binding-groove architecture. In some embodiments, a binding-groove architecture comprises one or more polypeptide lobes and a binding groove. In some embodiments, the polypeptide lobe of the binding-groove architecture each comprise one or more binding units as provided herein. In some embodiments, the binding groove of the binding-groove architecture comprises one or more hinge units as described herein. In some embodiments, the lobes, binding groove, or components thereof are connected by one or more linking units and/or linkers as described herein. Accordingly, in some embodiments, an engineered scaffold comprising a binding-groove architecture provided herein comprises one or more of: binding units, hinge units, linking units, linkers, or any combination thereof. Three-dimensional rendering of a binding groove architecture can be seen in FIGS. 33 and 34.

### Scaffold Proteins

An engineered scaffold protein provided herein, or a component thereof, can be derived from a scaffold protein. In some embodiments, each component of an engineered scaffold protein provided herein is independently derived from a scaffold protein. Therefore, each of a binding domain, binding unit, hinge unit, linking unit, immunoglobulin unit, epitope masking unit, and the like, is derived from a scaffold protein or a component thereof. Such a scaffold protein, or a component thereof, would serve as a wild-type counterpart or an experimental control for the corresponding component when describing comparable metrics herein.

In some embodiments, the engineered scaffold protein provided herein, or a component thereof, is derived from a scaffold protein described in TABLE 1. In some embodiments, the engineered scaffold protein is an engineered MHC II monomer able to bind to disordered regions in intact proteins. In some embodiments, the engineered scaffold protein is an engineered MHC I molecule able to bind disordered regions in intact proteins. In some embodiments, the engineered scaffold protein is an MHC-like protein engineered to bind disordered regions in intact proteins. In some embodiments, the engineered scaffold protein is an engineered zinc finger binding protein able to bind linear epitopes in intact proteins. In some embodiments, the engineered scaffold protein is an engineered caspase protein able to bind linear epitopes in intact proteins. In some embodiments, the engineered scaffold protein is an engineered protein ligase, including biotin ligase, sortase, subtilisin derived enzyme, etc. In some embodiments, the engineered scaffold protein is an engineered protease from TEV, enterokinase, thrombin, factor Xa, etc. In some embodiments, the engineered scaffold protein is an engineered ACT domain protein. In some embodiments, the engineered scaffold protein is an engineered kinase and/or phosphatase. In some embodiments, the engineered scaffold protein is an engineered chaperone protein.

### Genesis and Design

In some embodiments, an engineered scaffold protein is designed to bind to a target peptide described herein. As described herein, a scaffold protein may be selected as a binding protein, from which an engineered scaffold protein is derived from. Selection of the scaffold may be based on the type of molecule bound (e.g., DNA, RNA, or protein), known interactions (e.g., binding selectivity, strength, affinity, etc.) with the molecule, immunogenicity of the scaffold, function or biological activity of the scaffold, and the like. Upon selection of the scaffold protein, an amino acid sequence of an engineered scaffold protein may be generated to bind to a target peptide.

Accordingly, in some embodiments, also provided herein is a method of generating an amino acid sequence of an engineered scaffold protein which binds to a disordered region of a target peptide. In some embodiments, the method comprises selecting one or more scaffold protein sequences. Selection of one or more scaffold protein sequences may be through any suitable technique, such as mining from a metagenomic database. In some embodiments, to select more than one scaffold protein, a sequence homology search may be performed (e.g., protein domain searches, pairwise or HMM-based alignment).

In some embodiments, the method comprises evaluating the one or more scaffold protein sequences for a desired function, activity and/or characteristic. In some embodiments, evaluating the one or more scaffold protein sequences for a desired function, activity and/or characteristic comprises weighing factors relevant to ligand binding, immunogenicity, binding selectivity, binding frequency, binding speed, binding affinity, binding duration, function or biological activity, resistance to proteolytic cleavage, solubility, stability, half-life, and the like, or a combination thereof, of the scaffold protein(s) to generate amino acid sequence of an engineered scaffold protein predicted to have one or more enhanced or improved desired characteristic comprising: ligand binding, immunogenicity, binding selectivity, binding frequency, binding speed, binding affinity, binding duration, function or biological activity, resistance to proteolytic cleavage, solubility, stability, half-life, and the like, or combination thereof. Examples of such weighed factors comprises: the three-dimensional conformation, the individual protein domains, amino acid sequence, charge, polarity, hydrophobicity/hydrophilicity, and/or the acidity/baseness of certain amino acid residues (e.g., amino acid residues that interact or bind with the ligand), and the like, or any combination thereof, of the scaffold protein(s). In some embodiments, weighing one or more factors of a scaffold protein comprises: assigning a value to the one or more weighed factors based on an estimated probability of enhancing one or more desired characteristic, assigning a value to the one or more weighed factors and measuring the deviation of said value relative to a target value or threshold or to a value assigned to such a factor of a differing scaffold protein, or both. In some embodiments, the method further comprises predicting whether a generated amino acid sequence represents an engineered scaffold protein exhibiting an enhanced characteristic; whether one or more alteration of the one or more weighed factors improves said value relative to a target value or threshold or to a value assigned to such a factor of a differing scaffold protein; or both. A differing scaffold protein can be a naturally occurring scaffold protein, a WT counterpart, or a second scaffold protein where more than one scaffold protein is selected in methods described herein. In some embodiments, one or more alteration comprises one or more amino acid alteration.

In some embodiments, the method comprises selecting and evaluating one or more scaffold protein sequences as described herein, and engineering the scaffold protein sequence to bind to a linear epitope of a disordered region of a target peptide. In certain embodiments, an engineered scaffold protein sequence, such as the binding unit, or in some embodiments, the hinge unit, or both, of an engineered scaffold protein, is also engineered to have based on the estimated probability of having enhancements of one or more desired characteristics (e.g., enhanced ligand binding, immunogenicity, binding selectivity, binding frequency, binding speed, binding affinity, binding duration, function or biological activity, resistance to proteolytic cleavage, solubility, stability, half-life, and the like, or a combination thereof). In some embodiments, the method comprises selecting and evaluating one or more scaffold protein sequences as described herein, and engineering the scaffold protein sequence to bind to one or more post-translational modifications of a target peptide. Examples of post-translational modifications include: phosphorylation, glycosylation, acetylation, ubiquitination, proteolytic cleavage, nitrosylation, methylation, lipidation, etc. Further post-translation modifications are described throughout the present disclosure. In some embodiments, the method comprises selecting and evaluating one or more scaffold protein sequences as described herein, and engineering the scaffold protein sequence to bind to a target peptide wherein the target peptide is a therapeutic peptide. In some embodiments, the method comprises selecting and evaluating one or more scaffold protein sequences as described herein, and engineering the scaffold protein sequence to bind to a target peptide, wherein the target peptide is a ligand in one or more cell signalling pathways. In some embodiments, the method comprises selecting and evaluating one or more scaffold protein sequences as described herein, and engineering the scaffold protein sequence to be suitable or optimized for use with a molecular transporter as described herein.

Engineering of sequences of engineered scaffold proteins can be through suitable methods, such as computational, rational or directed evolution methods. Examples include: site directed mutagenesis, rational design, domain swapping, ancestral sequence reconstruction, relative to the corresponding scaffold protein(s). Accordingly, methods described herein generate engineered scaffold proteins which comprise one or more alterations or one or more amino acid alterations relative to a WT counterpart and/or the corresponding scaffold protein.

In some embodiments, the method comprising engineering and/or generating the amino acid sequence(s) of engineered scaffold protein(s) by performing the methods described herein. In some embodiments, the method further comprises engineering and/or generating an amino acid sequence based on the selected scaffold protein(s), the generated amino acid sequence representing an engineered scaffold protein predicted to have enhanced or improved ligand binding, immunogenicity, binding selectivity, binding frequency, binding speed, binding affinity, binding duration, function or biological activity, resistance to proteolytic cleavage, solubility, stability, half-life, and the like, or a combination thereof.

In some embodiments, evaluating the one or more scaffold protein sequences comprises manually evaluating the amino acid sequences of the one or more scaffold protein sequences. In some embodiments, the method comprises evaluating the one or more scaffold protein sequences in a machine learning model. In some embodiments, machine learning model used herein is trained to enhance the one or more desired characteristic of the scaffold protein. In some embodiments, a machine learning model used herein a generative model, a deep learning model, or the like.

In some embodiments, the method further comprises: a second iteration of evaluating one or more desired characteristics (e.g., ligand binding, immunogenicity, binding selectivity, binding frequency, binding speed, binding affinity, binding duration, function or biological activity, resistance to proteolytic cleavage, solubility, stability, half-life, and the like, or a combination thereof), of the generated engineered scaffold protein; weighing the one or more weighed factors (e.g., three-dimensional conformation, individual protein domains, charge, polarity, hydrophobicity/hydrophilicity, and/or the acidity/baseness of certain amino acid residues (e.g., amino acid residues that interact or bind with the ligand), and the like, or a combination thereof), of the generated engineered scaffold protein; or both; to inform further generation of engineered scaffold proteins. In some embodiments, the aforementioned method steps may be repeated more than once or multiple times.

In some embodiments, the method further comprises assaying the generated engineered scaffold protein. In some embodiments, the method further comprises assaying the generated engineered scaffold protein in silico, simulating data on ligand binding, immunogenicity, binding selectivity, binding frequency, binding speed, binding affinity, binding duration, function or biological activity, resistance to proteolytic cleavage, solubility, stability, half-life, and the like, or a combination thereof, and weighing the metrics against the prior predicted outcomes of the same. In some embodiments, the method further comprises assaying the generated engineered scaffold protein in an in vitro or in vivo assay, obtaining data on ligand binding, immunogenicity, binding selectivity, binding frequency, binding speed, binding affinity, binding duration, function or biological activity, resistance to proteolytic cleavage, solubility, stability, half-life, and the like, or a combination thereof, and weighing the data against the prior predicted outcomes of the same. In some embodiments, method further comprises evaluating the generated engineered scaffold protein in a machine learning model, predicting outcomes on ligand binding, immunogenicity, binding selectivity, binding frequency, binding speed, binding affinity, binding duration, function or biological activity, resistance to proteolytic cleavage, solubility, stability, half-life, and the like, or a combination thereof, and weighing the data against the prior predicted outcomes of the same.

Accordingly, in some embodiments, a method of obtaining an engineered scaffold protein or fusion protein thereof that binds to a target peptide is described herein. In some embodiments, said method comprises contacting a target peptide with an engineered scaffold protein, fusion protein thereof, nucleic acid encoding the same, host cell expressing the same, or library comprising any of the foregoing under conditions that allow an engineered scaffold protein: target peptide complex to form. In some embodiments, methods further comprise obtaining from the engineered scaffold protein: target peptide complex, the engineered scaffold protein scaffold that binds the target peptide, and preferably the engineered scaffold protein scaffold that has improved binding activity on the target peptide relative to the scaffold protein counterpart.

In some embodiments, a method of obtaining at least two engineered scaffold proteins that bind to one or more target peptides. In some embodiments, said method comprises contacting one or more target peptide with an engineered scaffold protein, fusion protein thereof, nucleic acid encoding the same, host cell expressing the same, or library comprising any of the foregoing under conditions that allow an engineered scaffold protein: target peptide complex to form. In some embodiments, the method further comprises engaging said engineered scaffold protein: target peptide complex with a crosslinking agent wherein the crosslinking of said engineered scaffold protein: target peptide complex elicits a detectable response. Examples of crosslinking agents include an antibody, an antibody fragment, a binding peptide, or an epitope tag. In some embodiments, the method further comprises obtaining from the complex, said engineered scaffold proteins that bind the one or more target peptide. In some embodiments, the two or more engineered scaffold proteins bind to the same epitope of a target peptide or to distinct epitopes of the target peptide. In some embodiments, methods of evaluating and/or methods of assaying described herein further comprise the evaluation of an engineered scaffold protein or analysis of an engineered scaffold protein as fused to a fusion partner and/or heterologous agent. Fusion partners and heterologous agents are further described herein.

In some embodiments, the results from in silico, in vitro, in vivo assays, and/or machine learning models can be used herein to inform further or other evaluation of scaffold protein(s) and/or generating future iterations of engineered scaffold proteins. In other embodiments, wherein the method comprises the use of a machine learning model, the method comprises utilizing the results from the in silico, in vitro, in vivo assays, and/or machine learning model described herein to retrain the machine learning model for further evaluations of scaffold proteins. In some embodiments, the machine learning model can be retrained in multiple iterations. In some embodiments, retraining the machine learning model comprises minimizing and/or optimizing one or more: functions, weights, parameters, variables, characteristics, and the like of the machine learning model. In some embodiments, also described herein are methods of generating an amino acid sequence of an engineered scaffold protein using the retrained machine learning model.

In some embodiments, the above method can be performed in a computer readable medium. Accordingly, in some embodiments, also described herein are systems for performing a method of generating an amino acid sequence of an engineered scaffold protein which binds to a disordered region of a target peptide. Such a system can comprise a computer readable medium comprising a computer readable memory that is capable of storing instructions for performing the methods described herein. Computer readable memory can be local (e.g., a hard drive) or online (e.g., cloud). For example, the computer readable memory can comprise instructions for evaluating scaffold protein(s), generating amino acid sequences of engineered scaffold protein(s), or both. In some embodiments, systems described herein are computer systems utilizing the computer readable medium as described herein, wherein the system further comprises a processor operatively coupled to the computer readable medium. In some embodiments, the processor is configured to execute the instructions to perform a method as described herein. The system can further comprise means for user input and output, such as a keyboard, monitor, and mouse.

In some embodiments, a system described herein can be configured to access a database, such as a local or online (e.g. cloud). Exemplary databases include protein structure databases, protein sequence databases, homology databases, nucleic acid sequence databases, and the like.

Upon execution of a method described herein, a system can further comprise information obtained by executing a method described herein. For example, upon execution of a method described herein, a system can comprise predicted outcomes from evaluating scaffold protein(s) and/or engineered scaffold protein(s). A system can comprise the amino acid sequences of engineered scaffold proteins generated from the performed methods as described herein. Further, a system can comprise the data and/or results generated from the in silico, in vitro and/or in vivo assays and/or machine learning models as described herein. Such systems can include means for transferring the information obtained by the methods described herein. In some instances, the systems can include means to transmit information obtained by the methods described herein into an external database (e.g. a local database or an online database).

### Epitope Masking Unit

In some embodiments, compositions, systems and methods provided herein further comprise an epitope masking unit or a use thereof. An epitope masking unit provided herein can at least partially occlude the binding domain of the engineered scaffold protein, thereby masking a binding epitope comprised on the surface of the binding domain. In certain embodiments, a binding domain comprises one or more amino acid alterations relative to the counterpart WT, in such embodiments, an epitope masking unit provided herein can harbor the one or more amino acid alterations from recognition by the immune system. Such an epitope masking unit can also function as an inhibitor of autoimmunity.

In some embodiments, an epitope masking unit can be any peptide which can non-covalently interact or bond with a binding domain as described herein. In some embodiments, an epitope masking unit is at least about 60%, about 65%, about 70%, about 75%, about 80%, about 85%, about 90%, about 95%, or about 100% antisense to one or more amino acids of a binding domain of an engineered scaffold protein as determined by corresponding sense-antisense amino acid pairing. In some embodiments, an epitope masking unit provided herein can be found in nature with the WT counterpart of an engineered scaffold protein. Examples are the use of a CLIP peptide, or a variant thereof, when using an MHC class II scaffold protein from which an engineered scaffold protein as provided herein is derived from.

In some embodiments, an epitope masking unit as described herein can also function to minimize off-target binding. An engineered scaffold protein provided herein, in some embodiments, be unbound by a target peptide. In such embodiments, an epitope masking unit has described herein can bind to a binding domain such that only peptides with a strong binding affinity can displace the epitope masking unit. In such embodiments, other off-target peptides would need to compete with the epitope masking unit in order to bind to the binding domain.

In some embodiments, an epitope masking unit can also function to stabilize a receptive conformation of an engineered scaffold protein provided herein such that the engineered scaffold protein maintains a conformation more suited for binding to a disordered region described herein.

An epitope masking unit as provided herein can be associated with an engineered scaffold protein described herein. An epitope masking unit can be attached to an engineered scaffold protein. For example, the epitope masking unit can be fused, linked, or covalently or non-covalently bound to an engineered scaffold protein. An epitope masking unit can be linked by a linker as described herein, such as a self-cleavable linker or a proteolytically cleavable. An epitope masking unit can be bonded to proximal cysteine or other proximal residues. An epitope masking unit can be attached at the C-terminal end, N-terminal end, or in an intermediary loop of the engineered scaffold protein.

### Fusion Partners

In some embodiments, compositions, systems and methods provided herein further comprise a fusion partner or a use thereof. Also provided herein is a fusion protein having an engineered scaffold protein as described herein and a fusion partner. In some embodiments, compositions, systems, and methods provided herein comprise two or more fusion partners, including 2, 3, 4, 5 or more fusion partners. In some embodiments, a fusion protein comprising multiple engineered scaffold proteins described herein is multispecific (e.g., bispecific). Such a multispecific fusion protein can include two, three, four, or more engineered scaffold proteins as well as one, two or more fusion partners.

Fusion partners provided herein comprise one or more of: a protein or a functional fragment thereof, an enzyme or a functional fragment thereof, an antibody or a functional fragment thereof, a compound or molecule which increases half-life of an engineered scaffold protein, a cytotoxic agent, a therapeutic agent, an immunologic agent, a detection agent, more than one of the foregoing, or any combination thereof. Examples of a fusion partner comprising a protein or a functional fragment thereof include a human serum albumin (HSA) or a domain thereof (e.g., a HSA binding domain), a fibrinogen or a domain thereof (e.g., a fibrinogen binding domain), complement component 1q (C1q), transferrin or a domain thereof (e.g., a transferrin binding domain), a molecular transporter (such as a cell penetrating peptide (CPP)), and the like. In some embodiments, a fusion partner comprises or is a molecular transporter. Examples of molecular transporters suitable as fusion partners or suitable to be comprised in a fusion partner are discussed in further detail en infra including in TABLE 13. Examples of a fusion partner comprising an enzyme or a functional fragment thereof include a ubiquitin ligase, trypsin, kinase, phosphatase, chaperone, and the like. Examples of a fusion partner comprising an antibody or a functional fragment thereof include a Fc region of an antibody. Examples of a fusion partner comprising a compound or molecule which increases half-life of an engineered scaffold protein include polymers (e.g., polyethylene glycol or PEGylation), carbohydrates, post-translational modifications (e.g., N-terminal glycosylation, and/or polymer mimetics), HSA binding domain, variant Fc domain, transferrin binding domain, fibrinogen binding domain, and the like. Examples of a fusion partner comprising a cytotoxic agent include a Fc region of an antibody, complement component 1q, a chemotherapeutic agent, and the like. Examples of a fusion partner comprising a therapeutic agent or therapeutic partner described herein include anti-cancer drugs, anti-inflammatories, anti-bacterials, anti-virals, cytokines, toxins, enzymes, neuroprotective agents, soluble factor traps, and the like. Examples of a fusion partner comprising an immunologic agent include a foreign antigen and the like. Examples of a fusion partner comprising a detection agent include a fluorescent molecule, a radioisotope, a reporter molecule, a detectable signal and the like. Other examples of suitable fusion partners are described in Zaman R, et. al., Current strategies in extending half-lives of therapeutic proteins. J Control Release. 2019 May 10; 301:176-189. doi: 10.1016/j.jconrel.2019.02.016. Epub 2019 Mar. 5. PMID: 30849445; and in Silver, et. al., Engineered antibody fusion proteins for targeted disease therapy, Trends in Pharmacological Sciences, Volume 42, Issue 12, 2021, Pages 1064-1081, ISSN 0165-6147, doi.org/10.1016/j.tips.2021.09.009. (sciencedirect.com/science/article/pii/S0165614721001899).

Exemplary fusion partners are set forth in TABLE 10.

Binding of the engineered scaffold protein or fusion protein thereof can modify specific biology/biochemistry of the target peptide, intracellular microenvironment comprising the target peptide, a cell expressing the target peptide, or extracellular environment the target peptide is localized to.

In some embodiments, the engineered scaffold protein or a fusion protein thereof as described herein, once bound to a target protein, acts as antagonist or inhibitor. An inhibitor as described herein, when used in reference to an engineered scaffold protein or fusion protein thereof refers to a protein that can hinder, restrain, or prevent the activity of a target protein once it binds to the target protein. Such an engineered scaffold protein or fusion protein thereof can function in various therapeutic modalities as described herein. In some embodiments, the engineered scaffold protein or fusion protein thereof, once bound to a target protein, acts as a degrader of the target protein by ubiquitination of the target protein. In some embodiments, the engineered scaffold protein or fusion protein thereof is conjugated to a drug that, once bound to a target protein, provides for the delivery of the drug to a cell expressing the target protein. In some embodiments, also provided herein is a fusion protein comprising an engineered scaffold protein as described herein and an Fc region of an antibody that, once bound to a target protein expressed by a cell, facilitates antibody-dependent cellular cytotoxicity (ADCC). In some embodiments, also provided herein is an engineered scaffold protein or a fusion protein thereof as described herein that, once bound to a target protein expressed by a cell, prevents ADCC. In some embodiments, also provided herein is a fusion protein comprising an engineered scaffold protein as described herein and a fusion partner that can bind complement component 1q (C1q) that, once bound to a target protein expressed by a cell, facilitates complement-dependent cytotoxicity (CDC). In some embodiments, also provided herein is an engineered scaffold protein or a fusion protein thereof as described herein that, once bound to a target protein expressed by a cell, prevents CDC. In some embodiments, also provided herein is an engineered scaffold protein or a fusion protein thereof as described herein that provides for the presentation of an antigen on an antigen presenting cell, thereby generating an immune response. Such an engineered scaffold protein or fusion protein thereof can be used as an infectious vaccine, a neurodegeneration vaccine, or a cancer neoepitope vaccine.

In some embodiments, the engineered scaffold protein or fusion protein thereof provides a target specific marker for use in the detection of a diagnostic marker of a disease. This includes the generation of or use of a panel of engineered scaffold proteins or fusion protein thereof for autoimmune diseases, different oncology indicators, and neurodegenerative diseases, etc. In some embodiments, the engineered scaffold protein or fusion protein thereof provides a target specific binder and/or reagent for in situ and alt-immunofluorescence detection. In some embodiments, the engineered scaffold protein or fusion protein thereof provides a target specific binder and/or reagent for protein fingerprinting and sequencing.

In some embodiments, an engineered scaffold protein or fusion protein thereof as provided herein is inhibitor or comprises inhibition activity, wherein the engineered scaffold protein or fusion protein thereof can function as an inhibitor of a target protein once it binds to the target protein and prevents a biological activity of that target protein from occurring. Such biological activity may include protein-protein interaction, protein-nucleic acid interaction, protein isomerization, protein post translational modification (e.g., phosphorylation, glycosylation, acetylation, ubiquitination, proteolytic cleavage, etc.), protein refolding, and protein aggregation, etc. Biological activity may also include a mechanism whereby one interaction partner may be inhibited from binding, thereby facilitating a different interacting partner to bind more efficiently and triggering a different biological activity. In this scenario, the therapeutic target can be a protein other than a target protein, either in the extracellular matrix or in the intracellular environment. By preventing biological activity, cell growth may be prevented, for example, by disruption of certain signaling pathways involved with apoptosis. By binding to targets that prevent immune cell activity, the immune system itself may destroy a diseased cell (e.g., a cancer cell) or a pathogenic cell.

In some embodiments, an engineered scaffold protein or a fusion protein thereof as provided herein, comprises an activity that is provided by a fusion partner as described herein.

In some embodiments, the engineered scaffold protein is fused to a trypsin enzyme and selectively binds to a specific disordered region on a target protein. Once bound, any Lysines or Arginines in the vicinity of the binding interphase of the target protein is cleaved by trypsin, thereby destroying the target protein. In some embodiments, the engineered scaffold protein is fused to a kinase or a phosphatase, and selectively binds to a specific disordered region on a target protein. Once bound, the kinase or phosphatase then preferentially phosphorylates residues in the vicinity of the binding site (in the case of a kinase) or de-phosphorylate (in the case of phosphatase). In some embodiments, the engineered scaffold protein is fused to an E3 ligase and selectively binds to a specific disordered region on a target protein. Once bound, the E3 ligase subsequently ubiquitinates the target protein in the vicinity of the binding site, thereby targeting the protein of interest for degradation via the proteasome. In some embodiments, the engineered scaffold protein is fused to an Fc region of an antibody, which acts as a fusion antibody molecule, where the engineered scaffold protein can serve the purpose of the antibody Fab domain that specifically binds to a known epitope of an antigen (i.e., the disordered region that the binder recognizes specifically). In each of the embodiments, the engineered scaffold protein acts as a homing agent that specifically targets a protein of interest and effects a biological change or activity in the protein of interest through the action of the fusion partner.

In some embodiments, provided herein is a fusion protein having an engineered scaffold protein as described herein and a fusion partner, wherein the fusion partner is a chaperone protein. Here, the engineered scaffold protein targets a specific disordered region of a misfolded protein, and the chaperone molecule acts in a directed manner to refold the misfolded protein. In emphysema, for instance, α1-antitrypsin is a protease inhibitor that is misfolded and accumulates in hepatocytes. α1-antitrypsin has several disordered loops in its predicted structure, one of which is ALVNYIFFKGK (SEQ ID NO: 925). By designing a binder to this disordered region and fusing a chaperone enzyme (e.g., Hsp70 or Hsp90), misfolded α1-anti-trypsin can be specifically targeted by the binder and the chaperone can fold the protein correctly.

In some embodiments, an engineered scaffold protein provided herein is a universal scaffold or a universal scaffold protein, wherein the engineered scaffold protein comprises a binding framework that encompasses a structural basis for designing and engineering multiple different artificial binding sites. The binding sites for such scaffold proteins can be loops or rigid structural domains in the scaffold protein. Such universal scaffold proteins may consist of a single polypeptide chain. Additionally, the universal scaffold protein can possess an intrinsic conformational stability and thermodynamic stability and be amenable to humanization. A universal scaffold protein can also be engineered to bind multiple different disordered regions in different proteins with high affinity and specificity. In other words, a universal scaffold protein can be modified to result in different engineered scaffold proteins with different binding properties (e.g., sequence specificity and/or affinity).

The half-lives of the engineered scaffold proteins or fusion proteins thereof disclosed herein may be extended by several techniques. In some embodiments, the half-lives of engineered scaffold proteins or fusion proteins thereof disclosed herein may be extended by fusion to a compound or molecule as described herein. For instance, the half-life of an engineered scaffold protein or a fusion protein thereof may be extended by chemically conjugating it to polymers, carbohydrates, post-translational modifications that include N-terminal glycosylation, and/or polymer mimetics, the overall protein size can be increased, thereby helping reduce renal clearance and prolonging the bioavailability of the biologic or protein. Such chemical modifications may also aid in preventing the action of other molecules or proteins that would otherwise degrade the engineered scaffold or fusion protein thereof.

Another strategy to improve half-life involves fusing the engineered scaffold or fusion protein thereof to an HSA binding domain, variant Fc domain, Transferrin binding domain, or fibrinogen domain. Fusion to any of these domains can increase the size of the protein, thereby also reducing the rate of renal clearance. Additionally, these fusion partners can all bind to the Fc-Rn receptor or CTLA and be subjected to receptor mediated endocytosis, therefore also increasing the half-life. See, e.g., Al-Qahatani et al (2019), Biomedicine & Pharmacotherapy, Zaman et al (2019), Journal of Controlled Release. Half-life may be also increased by fusion to an antibody ScFv region or PEGylation of the engineered scaffold or fusion protein thereof.

Multiple engineered scaffold proteins and/or fusion partners can be fused or linked together to make a multi-specific drug (e.g., a fusion protein). A multi-specific scaffold construct as described herein can refer to a construct encoding or containing an engineered scaffold protein described herein or a fusion protein thereof, which can target multiple proteins and inhibit all of the target proteins as an antagonist or work as an agonist for all of the target proteins or be an antagonist for a subset of target proteins and an agonist for a subset of the target proteins. Protein domains can be fused or linked together to generate an engineered scaffold protein. For example, one or more binding unit, hinge unit, immunoglobulin unit, epitope masking unit, molecular transporter, and/or linking unit may be fused or linked together. In another example, one or more alpha helix, beta sheet, immunoglobulin unit, or combinations thereof can be fused or linked together.

### Linkers

In some embodiments, an engineered scaffold protein described herein comprises one or more linkers. The linkers disclosed herein can be flexible, cleavable, or rigid, depending on the nature of the application. Further linkers can be used to modulate solubility, increase expression, improve biological activity, enable targeting, or alter Pk. See, e.g., Chen et al., Adv Drug Deliv Rev., 65 (10): 1357-69 (2013).

In some embodiments, an engineered scaffold protein provided herein comprises 1, 2, 3, 4, 5, 6, 7, 8, 9, 10 or more linkers. In some embodiments, an engineered scaffold protein provided herein does not comprise any linkers. In some embodiments, a peptide linker comprises 3-25 amino acids in length. In some embodiments, a linking unit described herein can be replaced by a linker as provided herein.

In some embodiments, a linker may be a peptide linker or a non-peptide linker. In some embodiments, a peptide linker comprises 1 to 30 amino acids in length. In some embodiments, a peptide linker comprises 1 amino acid, 2 amino acids, 3 amino acids, 4 amino acids, 5 amino acids, 6 amino acids, 7 amino acids, 8 amino acids, 9 amino acids, 10 amino acids, 11 amino acids, 12 amino acids, 13 amino acids, 14 amino acids, 15 amino acids, 16 amino acids, 17 amino acids, 18 amino acids, 19 amino acids, 20 amino acids, 21 amino acids, 22 amino acids, 23 amino acids, 24 amino acids, 25 amino acids, 26 amino acids, 27 amino acids, 28 amino acids, 29 amino acids, 30 amino acids in length or more.

Protein domains can be linked together by short peptide linkers that can be any one of several, but not limited to, the following: LE; (GGS)n n=1, 2, 3, 4, 5 (SEQ ID NO: 987); (GGGGS)n n=1, 2, 3, 4, 5-(SEQ ID NO: 973); (SSG)n n=1, 2, 3, 4, 5 (SEQ ID NO: 988); GGGGGG (SEQ ID NO: 974); GGGGGGGG (SEQ ID NO: 975); and Peptide1-nucleic acid-Peptide2. In some embodiments, Peptide1 is any peptide sequence of 5 or more amino acids with a functional group at the N terminus. In some embodiments, Peptide2 is any peptide sequence of 5 or more amino acids with a functional group at the C terminus of the peptide. In some embodiments, the two peptides are conjugated to each other by a nucleic acid.

In some embodiments, linkers described herein link a molecular transporter to an engineered scaffold protein, a fusion protein thereof, or any component of either. Examples of molecular transporters suitable to be linked to an engineered scaffold protein, a fusion protein thereof, or any component of either are discussed in further detail en infra, including at TABLE 13.

The constructs disclosed herein can be assembled in multiple ways. In some embodiments, the construct is Nterm-Binder1-Linker1-Binder2-Linker2-Binder3-Linker3 . . . -Linker (n−1)-Binder (n)-Cterm. Here, “Nterm” denotes the N terminus of the biologic (e.g., therapeutic protein), “Cterm” denotes the C terminus of the molecule, “Binder” denotes an engineered scaffold protein or other binding protein, but at least one engineered scaffold protein is present in the molecule. In some embodiments, the fusion protein containing two engineered scaffold proteins is bispecific. A bispecific is a particular example of two engineered scaffold proteins linked together in the above construct. Each linker can be any one of the linkers disclosed herein and any combination of linkers can be used to fuse the binder moieties together.

In some embodiments, described herein is a multi-specific scaffold construct or a use thereof. A multi-specific scaffold construct can target multiple proteins and inhibit all of the target proteins as an antagonist, work as an agonist for all of the target proteins or be an antagonist for a subset of target proteins and an agonist for a subset of the target proteins. A single domain or a subset of domains in the multispecific scaffold construct can also be used as a means of homing in on the target cell or target proteins, and the other domains in the therapeutic can work as inhibitors or agonists, or a combination of inhibitors and agonists.

A multi-specific scaffold construct can disrupt protein-protein interactions by simultaneously binding to multiple partners of the protein interaction network and disrupt critical cellular processes necessary for cellular growth and survival. A multi-specific therapeutic can also function to bring together several proteins together that may not be able to come together on their own and facilitate protein-protein interactions between all the binding partners by being a hub.

Exemplary linkers are set forth in TABLE 9.

### Molecular Transporters

In some embodiments, provided herein are systems of one or more engineered scaffold proteins and one or more molecular transporters. In some embodiments, compositions, systems and methods described herein comprise one or more molecular transporters or uses thereof. In some embodiments, engineered scaffold proteins provided herein comprise one or more molecular transporters. In some embodiments, a molecular transporter is an entity or moiety that is capable of passing through or crossing a physiological barrier, such as a biological membrane. In some embodiments, in systems of one or more engineered scaffold proteins and one or more molecular transporters the molecular transporter is capable of transporting the engineered scaffold protein through or across the physiological barrier. In some embodiments, a molecular transporter has the ability to translocate in vitro and/or in vivo the mammalian cell membranes and enter into cells, and directs an engineered scaffold protein or fusion protein thereof, to a desired cellular destination, e.g. into the cytoplasm (cytosol, endoplasmic reticulum, Golgi apparatus, etc.) or the nucleus. Accordingly, in some embodiments, molecular transporters can direct or facilitate penetration of an engineered scaffold protein or fusion protein thereof across a phospholipid, mitochondrial, endosomal or nuclear membrane. The molecular transporter can also direct an engineered scaffold protein or fusion protein thereof from outside the cell through the plasma membrane, and into the cytoplasm or to a desired location within the cell, e.g., the nucleus, the ribosome, the mitochondria, the endoplasmic reticulum, a lysosome, or a peroxisome. Alternatively, or in addition, the molecular transporter can direct an engineered scaffold protein or fusion protein thereof across the blood-brain, trans-mucosal, hematoretinal, skin, gastrointestinal and/or pulmonary barriers.

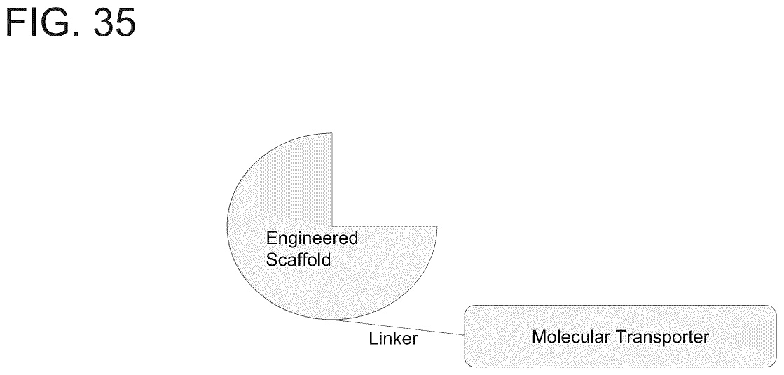

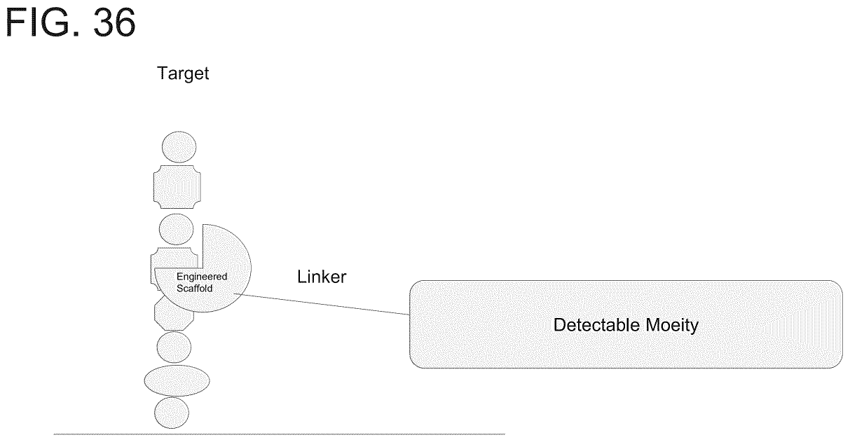

In some embodiments, compositions, systems and methods described herein comprise one or more, two or more, three or more, four or more, or five or more molecular transporters. In some embodiments, the molecular transporter is linked, conjugated or fused to the engineered scaffold protein, see for example FIG. 35. In some embodiments, the engineered scaffold protein is comprised in the molecular transporter.

Examples of molecular transporters include: cell penetrating peptides (CPPs) including cell penetrating poly(disulfides) (CPDs), protein-transduction domains (PTD) or membrane-translocation sequences (MTS), lipids, liposomes including cationic liposomes (e.g., as part of a cationic lipid and neutral co-lipid system), lipid nanoparticles, steroids, polyamines, nanotubes, nanoparticles including porous silicon nanoparticles (Si NPs) and polymeric nanoparticles (e.g., PEG-hydroxyethylene disulfide polymers), dendrimers (including guanidinium terminated dendrimers), peptoids (including N-substituted glycines), oligogcarbamates, HIV-binding peptides, HIV-1 Tat (HIV), Tat-derived peptides, Penetratin, VP22 derived or analog peptides, Pestivirus Erns, HSV, VP22 (Herpes simplex), MAP, KALA, PpT620, prolin-rich peptides, arginine-rich peptides, lysine-rich peptides, MPG-peptide(s), Pep-1, L-oligomers, Calcitonin peptide(s), Antennapedia-derived peptides (e.g., from Drosophila antennapedia), pAntp, pIsl, FGF, Lactoferrin, Transportan, Buforin-2, Bac715-24, SynB, SynB (1), pVEC, hCT-derived peptides, SAP, histones, modified or phosphorothioated single-stranded DNA (ssDNA), polymer-based materials (such as polyethylene glycol, polyethyleneimine, and polyboronic acids), protein nanocarriers (e.g., a Hex carrier), transmembrane antibodies, nanocapsules (NCs) including PEG coated hyaluronic acid nanoparticles (NPs), polymer NPs with glucose head groups, protein transduction domain mimics, anionic peptides, pyridylthiourea-modified polyethyleneimine (PEI), mutated lycotoxin, Sesbania mosaic virus like particles (VLPs), PEG- and arginine-capped lipids, microinjection and electroporation, poly(lactide-co-glycolic acid) (PLGA) NPs, etc. Further descriptions of molecular transporters can be found e.g., in Kayla C. Koch, Gregory N. Tew, Functional antibody delivery: Advances in cellular manipulation, Advanced Drug Delivery Reviews, Volume 192, 2023, 114586, the entire contents of which are incorporated herein by reference in its entirety.

Penetration across a biological membrane or a physiological barrier can be determined by various processes, for example by a cell penetration test having a first incubation step for the molecular transporter (e.g., a CPP) conjugated to an engineered scaffold protein and a marker in the presence of culture cells, followed by a fixating step, and then revelation of the presence of the marked engineered scaffold protein inside the cell. In another embodiment, the revelation step can be done with an incubation of the molecular transporter (e.g., a CPP) in the presence of labeled antibodies and directed against the molecular transporter (e.g., a CPP), followed by detection in the cytoplasm or in immediate proximity of the cell nucleus, or even within it, of the immunologic reaction between the molecular transporter's amino acid sequence and the labeled antibodies. Revelation can also be done by marking an amino acid sequence in the molecular transporter (e.g., a CPP) and detecting the presence of the marking in the cell compartments. Cell penetration tests are well known to those skilled in the art. However, for example a cell penetration test was described in the above-mentioned patent application No WO 97/02840.

The molecular transporter can be used in its natural or WT form or polymer form (dimer, trimer, etc.). In some embodiments, the molecular transporter comprises one or more modifications that can increase stability in vivo, bioavailability, and/or biological activity of the molecular transporter-engineered scaffold protein system. For example, in certain embodiments, amino acid based molecular transporters can have substitutions at 1, 2, 3, 4 or more residues. Modifications include,—N- and C-terminus modifications (e.g. to prevent exopeptidase degradation), C-terminal amidation and N-terminal acetylation increases peptide lipophilicity, cyclization by forming a disulfide bridge, alkylation of amide nitrogen to prevent endopeptidase degradation, introduction of non-natural amino acids to modify the recognition site of the endopeptidase (2-methylalanine, alpha-dialkylated glycine, oligocarbamate, oligourea, guanidino or amidino backbones), incorporation of non-genetically encoded amino acids (methylation, halogenation or chlorination of glycine or phenylalanine) into the amino acid sequence of the molecular transporter, replacement of some or even all the L-amino acids with their corresponding D-amino acid or beta-amino acid analogues, and the like. Exemplary molecular transporters can be seen in TABLE 13 below.

TABLE 13
Exemplary Molecular Transporters (e.g., CPPs)

| | | |
|---|---|---|
| Molecular | | SEQ |
| Transporter | Exemplary Molecular | ID |
| Type | Transporter | NO: |

Polyarginines

| | | |
|---|---|---|
| Poly Arginine-R₉ | RRRRRRRRR | 1014 |
| Poly Arginine-R₇ | RRRRRRR | 1015 |
| Buforin II | TRSSRAGLQFPVGRVHRLLRK | 1016 |
| DPV3 | RKKRRRESRKKRRRES | 1017 |
| DPV6 | GRPRESGKKRKRKRLKP | 1018 |
| DPV7 | GKRKKKGKLGKKRDP | 1019 |
| DPV7b | GKRKKKGKLGKKRPRSR | 1020 |
| DPV3/10 | RKKRRRESRRARRSPRHL | 1021 |
| DPV10/6 | SRRARRSPRESGKKRKRKR | 1022 |
| DPV1047 | VKRGLKLRHVRPRVTRMDV | 1023 |
| DPV1048 | VKRGLKLRHVRPRVTRDV | 1024 |
| DPV10 | SRRARRSPRHLGSG | 1025 |
| DPV15 | LRRERQSRLRRERQSR | 1026 |
| DPV15b | GAYDLRRRERQSRLRRRERQSR | 1027 |
| GALA | WEAALAEALAEALAEHLAEALA | 1028 |
| | EALEALAA | |

Haptotactic peptides

| | | |
|---|---|---|
| Cβ | KGSWYSMRKMSMKIRPFFPQQ | 1029 |
| preCγ | KTRYYSMKKTTMKIIPFNRL | 1030 |
| CaE | RGADYSLRAVRMKIRPLVTQ | 1031 |
| hCT(9-32) | LGTYTQDFNKFHTFPQTAIGVGAP | 1032 |
| HN-1 | TSPLNIHNGQKL | 1033 |
| Influenza virus | NSAAFEDLRVLS | 1034 |
| nucleoprotein | | |
| (NLS) | | |
| KALA | WEAKLAKALAKALAKHLAKALA | 1035 |
| | KALKACEA | |
| K-FGF | AAVALLPAVLLALLAP | 1036 |
| Ku70 | VPMLKPMLKE | 1037 |
| MAP | KLALKLALKALKAALKLA | 1038 |
| MPG | GALFLGFLGAAGSTMGAWSQP | 1039 |
| | KKKRKV | |
| MPM (IP/K-FGF) | AAVALLPAVLLALLAP | 1040 |
| N50 (NLS of | VQRKRQKLM | 1041 |
| NF-κB P50) | | |
| pISL | RVIRVWFQNKRCKDKK | 1042 |
| Prion mouse | MANLGYWLLALFVTMWTDVG | 1043 |
| PrPC₁₋₂₈ | LCKKRPKP | |
| pVEC | LLIILRRRIRKQAHAHSK | 1044 |
| SAP | VRLPPPVRLPPPVRLPPP | 1045 |
| SV-40 (NLS) | PKKKRKV | 1046 |
| SynB1 | RGGRLSYSRRRFSTSTGR | 1047 |
| SynB3 | RRLSYSRRRF | 1048 |
| SynB4 | AWSFRVSYRGISYRRSR | 1049 |
| Tat₄₇₋₆₀ | YGRKKRRQRRRPPQ | 1050 |
| Tat₄₇₋₅₇ | YGRKKRRQRRR | 1051 |
| Tat₄₉₋₅₇ | RKKRRQRRR | 1052 |
| Penetratin | RQIKIWFQNRRMKWKK | 1053 |
| (Antennapedia) | | |
| Short Penetratin | RRMKWKK | 1054 |
| Pep-1 | KETWWETWWTEWSQPKKKRKV | 1055 |
| Pep-7 | SDLWEMMMVSLACQY | 1056 |
| Transportan | GWTLNSAGYLLGKINLKALAALAKKIL | 1057 |
| Transportan 10 | AGYLLGKINLKALAALAKKIL | 1058 |
| Transportan | GWTLNSAGYLLG | 1059 |
| derivatives: | INLKALAALAKKIL | 1060 |
| VP22 | DAATATRGRSAASRPTERPRAPARS | 1061 |
| | ASRPRRPVD | |
| VT5 | DPKGDPKGVTVTVTVTVTGKGDPKPD | 1062 |
| [Dmt¹]DALDA | Dmt-DRFK | 1063 |
| Nuclear | SKKKKIKV | 1064 |
| Localization | | |
| Sequences | GRKRKKRT | 1065 |

### Vectors

Compositions, systems and methods described herein comprise one or more vectors or uses thereof. In some embodiments, one or more vectors used herein comprises one or more nucleotide sequences encoding one or more components of a composition or method described herein. In some embodiments, one or more vectors used herein is conjugated to or otherwise attached to one or more components of a composition or method described herein. In some embodiments, one or more components comprise one or more engineered scaffold proteins, fusion partners, linkers, fusion proteins, target peptides, molecular transporters, or functional fragments thereof. In some embodiments, where more than one vector is used, compositions, systems and methods described herein can comprise a library of vectors, each vector encoding or conjugated to one or more component of a composition or method as described herein. In some embodiments, components described herein are encoded by or conjugated to the same vector, or each component is encoded by or conjugated to a different vector, or combinations thereof.

In some embodiments, vectors described herein comprise or encode one or more regulatory elements. Such regulatory elements can be operably linked to a nucleotide sequence encoding an engineered scaffold protein, fusion partner, linker, fusion protein, target peptide, molecular transporter, or functional fragment thereof, or more than of the foregoing. Regulatory elements can include transcriptional and translational control sequences, such as promoters, enhancers, polyadenylation signals, terminators, and protein degradation signals, that provide for and/or regulate transcription of a non-coding sequence or a coding sequence and/or regulate translation of an encoded polypeptide. In some embodiments, a vector comprises or encodes for one or more additional elements, such as, for example, replication origins, antibiotic resistance (or a nucleic acid encoding the same), a tag (or a nucleic acid encoding the same), and selectable markers. In some embodiments, a vector comprises or encodes for one or more elements, such as, for example, ribosome binding sites, and RNA splice sites. In general, vectors provided herein comprise at least one promotor or a combination of promoters driving expression or transcription of engineered scaffold proteins, fusion partners, fusion proteins, target peptides or functional fragments thereof as described herein.

Vectors can be expression vectors. Vectors can be a viral vector, such as an AAV vector or a lentiviral vector, or a non-viral vector, such as a lipid, lipid particle, cell-penetrating peptide, or mRNA.

The engineered scaffold or fusion protein thereof disclosed herein may be used for therapeutic purposes by delivery of the engineered scaffold or fusion protein thereof to a target cell. Intracellular delivery of such therapeutics can be by AAV vector, lentiviral vector, cell-penetrating peptide or as mRNA.

The engineered scaffold protein or fusion protein thereof may be used for a polypeptide display library. In some embodiments, a polypeptide display library provided herein comprises an engineered scaffold protein or fusion thereof expressed and displayed by a vector described herein. In some embodiments, the engineered scaffold protein or fusion protein or fusion thereof is comprised in an expression vector and delivered to a host cell for expression. Accordingly, in some embodiments, provided herein is a host cell comprising an engineered scaffold protein or fusion protein described herein. In some embodiments, a polypeptide display library provided herein displays an engineered scaffold protein or fusion thereof on the surface of a virus or yeast, or displayed as a ribosome or RNA conjugated protein molecule.

In some embodiments, also provided herein are isolated nucleic acid molecules encoding an engineered scaffold protein described herein, a fusion thereof, one or more components of the following, or combinations thereof. In some embodiments, an expression vector can be operably linked to an isolated nucleic acid molecule provided herein. Accordingly, provided herein are compositions or methods comprising an isolated nucleic acid molecule encoding an engineered scaffold protein described herein, a fusion thereof, one or more components of the following, or combinations thereof, and in some embodiments, as operably linked to an expression vector.

### Target Peptides

Disclosed herein are compositions, systems and methods that comprise a target peptide or a use thereof. In some embodiments, an engineered scaffold protein or fusion protein thereof as described herein interacts with or binds to a target peptide. In some embodiments, interacting or binding with a target peptide results in the detection of the target peptide or results in inducing a biological or therapeutic activity. Accordingly, also disclosed herein are compositions, systems and methods for detecting a target peptide or inducing a biological or therapeutic effect.

A target peptide described herein can be part of a polypeptide, larger protein or protein complex. Accordingly, when referring to a target peptide that is comprised in or comprised with a polypeptide, protein or protein complex, reference is also made to the target polypeptide, target protein, and/or target protein complex. In some embodiments, reference to a target peptide also refers to a nucleic acid (i.e., a DNA or RNA molecule) which encodes the target peptide. Accordingly, in some embodiments, compositions, systems and methods described herein comprise a nucleic acid encoding a target peptide as described herein. In some embodiments, a target peptide may be referred to herein as a target ligand.

In some embodiments, a target peptide comprises 2 to 50, 3 to 40, or 4 to 30 amino acids in length. In some embodiments, a target peptide comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50 amino acids. In some embodiments, a target peptide comprises at least 4, at least 15, or at least 30 amino acids.

In some embodiments, a target peptide is comprised in a polypeptide, protein, or protein complex. In some embodiments, the polypeptide, protein or one protein within a protein complex comprising the target peptide can be greater than about 30 amino acids in length. In some embodiments, the target peptide is comprised in a polypeptide, protein, or protein complex, wherein the polypeptide, protein, or one protein within a protein complex is greater than about 30 amino acids in length. In some embodiments, the polypeptide, protein, or one protein of a protein complex comprising a target peptide is about 30 amino acids, 40 amino acids, 50 amino acids, 60 amino acids, 70 amino acids, 80 amino acids, 90 amino acids, 100 amino acids, 110 amino acids, 120 amino acids, 130 amino acids, 140 amino acids, 150 amino acids, 160 amino acids, 170 amino acids, 180 amino acids, 190 amino acids, 200 amino acids, 210 amino acids, 220 amino acids, 230 amino acids, 240 amino acids, 250 amino acids, 260 amino acids, 270 amino acids, 280 amino acids, 290 amino acids, 300 amino acids, 310 amino acids, 320 amino acids, 330 amino acids, 340 amino acids, 350 amino acids, 360 amino acids, 370 amino acids, 380 amino acids, 390 amino acids, 400 amino acids, 410 amino acids, 420 amino acids, 430 amino acids, 440 amino acids, 450 amino acids, 460 amino acids, 470 amino acids, 480 amino acids, 490 amino acids, 500 amino acids, or greater, in length.

In some embodiments, the polypeptide, protein or one protein of a protein complex comprising the target peptide can be greater than about 100 daltons in weight. In some embodiments, the target peptide is comprised in a polypeptide, protein, or protein complex, wherein the polypeptide, protein, or one protein of a protein complex is greater than about 100 daltons, 1000 daltons, 2000 daltons, 4000 daltons, 6000 daltons, 8000 daltons, 10000 daltons, 12000 daltons, 14000 daltons, 16000 daltons, 18000 daltons, 20000 daltons, 22000 daltons, 24000 daltons, 26000 daltons, 28000 daltons, 30000 daltons, 32000 daltons, 34000 daltons, 36000 daltons, 38000 daltons, 40000 daltons, 42000 daltons, 44000 daltons, 46000 daltons, 48000 daltons, 50000 daltons, 52000 daltons, 54000 daltons, 56000 daltons, 58000 daltons, 60000 daltons, 62000 daltons, 64000 daltons, 66000 daltons, 68000 daltons, 70000 daltons, 72000 daltons, 74000 daltons, 76000 daltons, 78000 daltons, 80000 daltons, 82000 daltons, 84000 daltons, 86000 daltons, 88000 daltons, 90000 daltons, 92000 daltons, 94000 daltons, 96000 daltons, 98000 daltons, 100000 daltons, or greater in weight.

In some embodiments, where a target peptide is comprised in a polypeptide, protein or protein complex, an engineered scaffold protein described herein can be engineered to be multispecific (e.g., bispecific or trispecific)-binding to multiple regions of the polypeptide, protein or protein complex. Regions can be disordered, ordered, or combinations thereof.

In some embodiments, a target peptide described herein is comprised in a cell. In some embodiments, a target peptide described herein is comprised in a population of cells. In some embodiments, a target peptide described herein is comprised in a naturally occurring cell, a eukaryotic cell, a prokaryotic cell, a plant cell, a fungal cell, an animal cell, cell of an invertebrate, a fly cell, a cell of a vertebrate, a mammalian cell, a primate cell, a non-human primate cell, a human cell, a living cell, a non-living cell, a modified cell, a derived cell, a non-naturally occurring cell, or any combination thereof. In some embodiments, a cell comprising a target peptide described herein is comprised in a eukaryotic cell. In some embodiments, a target peptide described herein is comprised in a population of: naturally-occurring cells, eukaryotic cells, prokaryotic cells, plant cells, fungal cells, animal cells, cells of an invertebrate, fly cells, cells of vertebrate, mammalian cells, primate cells, non-human primate cells, human cells, living cells, non-living cells, modified cells, derived cells, non-naturally occurring cells, or any combination thereof. In some embodiments, a cell comprising a target peptide described herein is comprised in a population of eukaryotic cells.

In some embodiments, a target peptide described herein is isolated from a cell. In some embodiments, a target peptide described herein is isolated from any one of: a naturally occurring cell, a eukaryotic cell, a prokaryotic cell, a plant cell, a fungal cell, an animal cell, cell of an invertebrate, a fly cell, a cell of a vertebrate, a mammalian cell, a primate cell, a non-human primate cell, a human cell, a living cell, a non-living cell, a modified cell, a derived cell, and a non-naturally occurring cell. In some embodiments, the target peptide is isolated from a population of cells. In some embodiments, a target peptide described herein is isolated from a population of: naturally-occurring cells, eukaryotic cells, prokaryotic cells, plant cells, fungal cells, animal cells, cells of an invertebrate, fly cells, cells of vertebrate, mammalian cells, primate cells, non-human primate cells, human cells, living cells, non-living cells, modified cells, derived cells, non-naturally occurring cells, or any combination thereof. In some embodiments, a target peptide described herein is comprised in a sample. In some embodiments, a target peptide described herein is comprised in a sample obtained or harvested from an organism. In some embodiments, a target peptide described herein is comprised in a sample obtained or harvested from: a eukaryotic organism, a prokaryotic organism, a plant, a fungal organism, a bacterium, a virus, an animal, an invertebrate, a fly, a vertebrate, a mammalian organism, a mouse, a primate, a non-human primate, a human.

In some embodiments, a target peptide described herein, or a cell or organism comprising the target peptide, is comprised in a system for detecting target peptides, such as a kit as described herein. In some embodiments, a target peptide described herein is comprised in a device for detecting target peptides. In some embodiments, a target peptide is naturally-occurring or comprises one or more modifications, for example modifications for use in a system or method described herein. Such modifications include fusion or conjugation to a detection agent as described herein.

In some embodiments, a target peptide described herein is comprised in an or is an extracellular protein. In some embodiments, a target peptide described herein is membrane protein. In some embodiments, a target peptide described herein is comprised in a or is a G protein-coupled receptor (GPCR), an ion channel, or a secreted protein. In some embodiments, a target peptide described herein is comprised in or is any of the proteins set forth in TABLE 7, or a portion thereof.

In some embodiments, a target peptide described herein is associated with a disease or disorder. In some embodiments, a target peptide described herein is encoded by a nucleic acid that is associated with a disease or disorder. In some embodiments, a target peptide described herein is comprised in a cell that is associated with a disease or disorder. In some embodiments, a target peptide described herein is comprised in an organism that is afflicted with a disease or disorder. In some embodiments, a disease or disorder described herein is a genetic disease or disorder, pathogenetic disease or disorder, infectious disease or disorder, communicable disease or disorder, immunologic disease or disorder, a mutagenic disease or disorder, or any combination thereof. In some embodiments, a disease or disorder described herein is associated with any of the target proteins set forth in TABLE 7. Also disclosed herein are methods of treating, preventing, or inhibiting a disease or disorder.

In some embodiments, an engineered scaffold protein or fusion protein thereof as described herein can interact with or bind a disordered region of a target peptide. In some embodiments, an engineered scaffold protein or fusion protein thereof as described herein interacts with or binds a linear epitope present in a disordered region of a target peptide. In some embodiments, a target peptide can be an intrinsically disordered peptide. In some embodiments, a target peptide may comprise an ordered structure, but still also comprise a disordered region.

In some embodiments, an engineered scaffold protein or fusion protein thereof as provided herein can interact with or bind to an ordered region of a target peptide. In some embodiments, an engineered scaffold protein or fusion protein thereof as provided herein can interact with or bind to an ordered region and a disordered region of a target peptide, either at the same binding domain or in different binding domains. Accordingly, engineered scaffold proteins or fusion proteins described herein that can be engineered as described herein to bind to disordered regions can also be engineered to bind to ordered regions.

### Disordered Region

In some embodiments, an engineered scaffold protein described herein can be engineered to interact with or bind to a disordered region of a target peptide as described herein. In some embodiments, the disordered region can be fully disordered or partially disordered. Once a target peptide is selected, disordered regions can be determined from Uniprot, or another sequencing database, and a disordered epitope comprising the complete region, or a subset of that disordered region, may be used for engineered scaffold protein development in accordance with any of the methods and examples disclosed herein.

In some embodiments, a disordered region of a target peptide can have an extended conformation that an engineered scaffold protein described herein can bind to. In some embodiments, an engineered scaffold protein described herein is engineered to interact with or bind to a disordered region of a target peptide comprising an extended conformation. In some embodiments, an extended conformation of a disordered region is a linear conformation or linear sequence.

In some embodiments, a disordered region is comprised in a loop, a C terminal tail, or an N terminal tail of a target peptide described herein. In some embodiments, an engineered scaffold protein described herein is engineered to interact with or bind to a loop, a C terminal tail, or an N terminal tail of a target peptide described herein. In some embodiments, an engineered scaffold protein described herein is engineered to interact with or bind to a disordered region comprised in a loop, a C terminal tail, or an N terminal tail of a target peptide described herein. In some embodiments, an engineered scaffold protein described herein is engineered to interact with or bind to a linear sequence of a disordered region comprised in a loop, a C terminal tail, or an N terminal tail of a target peptide described herein.

Disordered regions of a target peptide described herein can have known biological functions. In some embodiments, an engineered scaffold protein described herein is designed to bind to a target peptide thereby inducing, altering or inhibiting the biological functions of the disordered region.

In some embodiments, a disordered region of a target peptide described herein can function as a recognition site for enzyme active sites, or as a ligand site which is recognized by the binding surface of a protein partner or partners. A protein partner(s) as described herein refers to a polypeptide which can recognize and interact with a ligand site of a target peptide. In some embodiments, an engineered scaffold protein described herein is engineered to interact with or bind to a disordered region that functions as a recognition site for enzyme active sites, or as a ligand site which is recognized by the binding surface of a protein partner or partners. In some embodiments, binding of an engineered scaffold protein to a disordered region functioning as a recognition site can induce structural modification, proteolytic cleavage and post-translational modification removal or addition of the target peptide as described in further detail herein. In some embodiments, binding of an engineered scaffold protein to a disordered region functioning as a ligand site can induce complex promoting, docking, and targeting or trafficking of the protein partner(s) as described in further detail herein. In some embodiments, binding of an engineered scaffold protein to a target peptide can activate a disordered region functioning as a recognition site or as a ligand site thereby inducing the aforementioned activity. In some embodiments, binding of an engineered scaffold protein to a target peptide can occlude a disordered region functioning as a recognition site or as a ligand site thereby inhibiting the aforementioned activity.

In some embodiments, a disordered region of a target peptide can function as a promoter of protein-protein interactions. In some embodiments, an engineered scaffold protein described herein is engineered to interact with or bind to a disordered region that functions as a promoter of protein-protein interactions. In some embodiments, such disordered regions comprise preformed structural elements (PSEs), molecular recognition features (MoRFs), molecular recognition (MoREs), or prestructured motifs (PreSMos). In some embodiments, disordered regions comprising MoRFs undergo disorder-to-order transitions upon binding their interaction partners (i.e., folding upon binding). An interaction partner can be any protein that binds to a disordered region that is functioning as a promoter of protein-protein interactions. In some embodiments, disordered regions comprising PSEs or MoRFs can function as initial contact points for interaction events, wherein conformational selection of preformed elements and induced folding occur. In some embodiments, binding of an engineered scaffold protein to a target peptide can activate a disordered region functioning as a promoter of protein-protein interactions thereby inducing the aforementioned activity. In some embodiments, binding of an engineered scaffold protein to a target peptide can occlude a disordered region functioning as a promoter of protein-protein interactions thereby inhibiting the aforementioned activity.

In some embodiments, a disordered region of a target peptide can function as a DNA binder, RNA binder, or protein binder. In some embodiments, binding of an engineered scaffold protein to a target peptide can activate a disordered region functioning as a DNA, RNA, or protein binder thereby inducing the aforementioned activity. In some embodiments, binding of an engineered scaffold protein to a target peptide can occlude a disordered region functioning as a DNA, RNA, or protein binder thereby inhibiting the aforementioned activity.

In some embodiments, a disordered region comprises consecutive amino acids that comprise the complete sequence or a subset of the sequences enumerated that are at least 4, at least 5, at least 6, at least 7, at least 8, at least 9, at least 10, at least 12, at least 13, at least 14, at least 15, at least 16, at least 17, at least 18, at least 19, at least 20, at least 30, at least 40, or at least 50 consecutive amino acids of a target peptide.

From the selected target peptide sequences, disordered regions can be determined from Uniprot and a disordered epitope comprising the complete region, or a subset of that disordered region, may be used for engineered scaffold protein development in accordance with any of the methods and examples disclosed herein. Development of such an engineered scaffold protein may also be utilized for any of the applications disclosed herein, including vaccine development, therapeutic development, immunomodulation/suppression, detection, and diagnostics.

In embodiments where more than one engineered scaffold protein or fusion protein thereof is used, such engineered scaffold protein or fusion protein can recognize the same linear epitope or distinct linear epitopes.

Non-limiting examples of linear epitopes and disordered regions of target peptides are set forth in TABLE 7.

### Pharmaceutical Compositions

Disclosed herein, in certain embodiments, are pharmaceutical compositions comprising an engineered scaffold protein described herein and a carrier thereof for administration in a subject.

In certain embodiments, the pharmaceutically acceptable compositions comprise a therapeutically-effective amount of one or more of an engineered scaffold protein, formulated together with one or more pharmaceutically acceptable: carriers (additives) and/or diluents. In some embodiments, when an engineered scaffold protein herein are administered as pharmaceuticals, to humans and animals, they can be given per se or as a pharmaceutical composition containing, for example, 0.1 to 99%, or 10 to 30% of an engineered scaffold protein in combination with a pharmaceutically acceptable carrier.

A pharmaceutical composition of the present disclosure can be delivered, e.g., subcutaneously or intravenously with a standard needle and syringe or a pen delivery device. The injectable preparations may include dosage forms for intravenous, subcutaneous, intracutaneous and intramuscular injections, drip infusions, etc. The injectable preparations may be prepared, e.g., by dissolving, suspending or emulsifying an engineered scaffold protein herein in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant (e.g., polysorbate 80, HCO-50 (polyoxyethylene (50 mol) adduct of hydrogenated castor oil)), etc. As the oily medium, there are employed, e.g., sesame oil, soybean oil, etc., which may be used in combination with a solubilizing agent such as benzyl benzoate, benzyl alcohol, etc. The injection thus prepared can be filled in an appropriate ampoule.

Compositions of the present disclosure can be in the form of, for example, granules, powders, tablets, capsules, syrup, suppositories, injections, emulsions, elixirs, suspensions or solutions. The amount of an engineered scaffold protein disclosed herein contained can be about 5 to about 500 mg per dosage form in a unit dose. In one embodiment, an engineered scaffold protein can be contained in about in about 5 to about 100 mg, for example for a parental dosage form. In other embodiments, an engineered scaffold protein can be contained in about 10 to about 250 mg for the other dosage forms.

For example, oral, buccal, and sublingual administration, powders, suspensions, granules, tablets, pills, capsules, gelcaps, and caplets can be used as solid dosage forms. These can be prepared, for example, by mixing the engineered scaffold protein, with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. In some embodiments, pharmaceutical formulations and medicaments may be prepared as liquid suspensions or aqueous solutions, for example, using a sterile liquid, such as, but not limited to, an oil, water, an alcohol, or any combination of these. In some embodiments, pharmaceutical compositions can be prepared in a lyophilized form. The lyophilized preparations can comprise a cryoprotectant known in the art. The term “cryoprotectants” as used herein generally includes agents, which provide stability to the protein from freezing-induced stresses. Examples of cryoprotectants include polyols such as, for example, mannitol, and include saccharides such as, for example, sucrose, as well as including surfactants such as, for example, polysorbate, poloxamer or polyethylene glycol, and the like. Cryoprotectants also contribute to the tonicity of the formulations. Pharmaceutically suitable surfactants, suspending agents, emulsifying agents, may be added for oral or par-enteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, iso-propyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

For nasal administration, the pharmaceutical formulations and medicaments may be a spray or aerosol containing an appropriate solvent(s) and optionally other compounds such as, but not limited to, stabilizers, antimicrobial agents, antioxidants, pH modifiers, surfactants, bio-availability modifiers or any combination of these. A propellant for an aerosol formulation may include compressed air, nitrogen, carbon dioxide, or a hydrocarbon based low boiling solvent.

Injectable dosage forms generally include aqueous suspensions or oil suspensions which may be prepared using a suitable dispersant or wetting agent and a suspending agent. Injectable forms may be in solution phase or in the form of a suspension, which can be prepared with a solvent or diluent. Acceptable solvents or vehicles include sterilized water, Ringer's solution, or an isotonic aqueous saline solution. Alternatively, sterile oils may be employed as solvents or suspending agents. In some embodiments, the oil or fatty acid is non-volatile, including natural or synthetic oils, fatty acids, mono-, di- or tri-glycerides.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers or any combination of these.

For rectal administration, the pharmaceutical formulations and medicaments may be in the form of a suppository, an ointment, an enema, a tablet or a cream for release of compound in the intestines, sigmoid flexure and/or rectum. Rectal suppositories are prepared by mixing one or more compounds herein with acceptable vehicles, for example, cocoa butter or polyethylene glycol, which is present in a solid phase at normal storing temperatures, and present in a liquid phase at those temperatures suitable to release a drug inside the body, such as in the rectum. Oils may also be employed in the preparation of formulations of the soft gelatin type and suppositories. Water, saline, aqueous dextrose and related sugar solutions, and glycerols may be employed in the preparation of suspension formulations which may also contain suspending agents such as pectins, carbomers, methyl cellulose, hydroxypropyl cellulose or carboxymethyl cellulose, as well as buffers and preservatives.

The concentration of an engineered scaffold protein in these compositions can vary widely, e.g., from less than about 10%, least about 25% to as much as 75% or 90% by weight and will be selected primarily by fluid volumes, viscosities, etc., in accordance with the particular mode of administration selected.

In some embodiments, pharmaceutical compositions comprising an engineered scaffold protein described herein can be formulated using one or more physiologically acceptable carriers including excipients and auxiliaries which facilitate processing of the active compounds into preparations which can be used pharmaceutically. Proper formulation is dependent upon the route of administration chosen.

Pharmaceutical compositions are optionally manufactured such as, by way of example only, by means of mixing, dissolving, granulating, dragee-making, levigating, emulsifying, encapsulating, entrapping or compression processes.

In certain embodiments, compositions may also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range. In other embodiments, compositions may also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In some embodiments, sustained-release preparations can be used. Suitable examples of sustained-release preparations include semipermeable matrices of solid hydrophobic polymers containing an engineered scaffold protein of the present disclosure, in which the matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides, copolymers of L-glutamic acid and y ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they can denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thiodisulfide interchange, stabilization can be achieved by modifying sulfhydryl residues, lyophilizing from acidic solutions, controlling moisture content, using appropriate additives, and developing specific polymer matrix compositions. In certain situations, the pharmaceutical composition can be delivered in a controlled release system. In one embodiment, a pump may be. In another embodiment, polymeric materials can be used. In yet another embodiment, a controlled release system can be placed in proximity of the composition's target, thus requiring only a fraction of the systemic dose.

In some embodiments, an engineered scaffold protein can be administered with one or more agents capable of promoting penetration of the engineered scaffold protein across the blood-brain barrier. In some embodiments, an engineered scaffold protein can be linked with a viral vector, e.g., to render an engineered scaffold protein more effective or increase transport across the blood-brain barrier. For example, delivery of agents can be by administration of an adenovirus vector to motor neurons in muscle tissue. Delivery of vectors directly to the brain, include but are not limited to the striatum, the thalamus, the hippocampus, or the substantia nigra.

In embodiments, an engineered scaffold protein can be linked or conjugated with agents that provide desirable pharmaceutical or pharmacodynamic properties. In some embodiments, an engineered scaffold protein can be coupled to a substance that promotes penetration or transport across the blood-brain barrier, e.g., an antibody to the transferrin receptor. In some embodiments, osmotic blood brain barrier disruption can be assisted by infusion of sugars, e.g., meso erythritol, xylitol, D(+) galactose, D(+) lactose, D(+) xylose, dulcitol, myo-inositol, L(−) fructose, D(−) mannitol, D(+) glucose, D(+) arabinose, D(−) arabinose, cellobiose, D(+) maltose, D(+) raffinose, L(+) rhamnose, D(+) melibiose, D(−) ribose, adonitol, D(+) arabitol, L(−) arabitol, D(+) fucose, L(−) fucose, D(−) lyxose, L(+) lyxose, and L(−) lyxose, or amino acids, e.g., glutamine, lysine, arginine, asparagine, aspartic acid, cysteine, glutamic acid, glycine, histidine, leucine, methionine, phenylalanine, proline, serine, threonine, tyrosine, valine, and taurine. In some embodiments, the composition can be encapsulated in glucose-coated polymeric nanocarriers.

The compositions herein may be administered alone or in combination with another therapeutic. The additional therapeutic may be administered prior, concurrently, consecutively, or subsequently to the administration of the composition.

The compositions disclosed herein, comprising an engineered scaffold protein, described herein, can also contain more than one active agent as necessary for the particular indication being treated, such as those with complementary activities that do not adversely affect each other. For example, the composition can further comprise an anti-inflammatory, a therapeutic protein, a steroid, an analgesic, a non-steroidal anti-inflammatory, a corticosteroid, an immune system modulator, an additional engineered scaffold protein, more than one of the foregoing, or any combination thereof.

### Methods of Use

Provided herein are compositions, systems and methods for binding a target peptide comprising the use of an engineered scaffold protein or a fusion protein thereof described herein. Binding of a target peptide by an engineered scaffold protein or a fusion protein thereof described herein is capable of inducing a biological or therapeutic activity. For example, binding of a target peptide by an engineered scaffold protein or a fusion protein thereof described herein can result in: activation of a binding site; inhibition or occlusion of a binding site; induction of conformational biasing; prevention of nuclear localization; disruption of membrane binding; modulation of macromolecule interactions; occlusion or sequestering of post-translational modifications (PTMs); prevention of pro-protein processing; induction of cell degradation; blockade of substrate recruitment; and the like; or any combination thereof.

Also provided herein are compositions comprising an engineered scaffold protein or a fusion protein thereof described herein, wherein the engineered scaffold protein or the fusion protein thereof is bound, fused, conjugated, or otherwise attached to a therapeutic partner. Therefore, in some embodiments, binding of a target peptide by an engineered scaffold protein or a fusion protein thereof described herein can deliver or localize the therapeutic partner to the target peptide, a cellular microenvironment the target peptide is comprised in, a cell comprising the target peptide, or an extracellular environment which the target peptide is adjacent to or comprised in. Accordingly, in some embodiments, binding of a target peptide by an engineered scaffold protein or a fusion protein thereof described herein is capable of inducing a biological or therapeutic activity effected by a therapeutic partner described herein. For example, binding of a target peptide by an engineered scaffold protein or a fusion protein thereof described herein can result in: inducing cell death; inducing proteolytic cleavage; modulation of PTMs; inducing de-phosphorylation; inducing protein isomerization; inducing protein disaggregation and/or refolding; modulation of ATPase and/or GTPase activity; inducing a immune response; and the like; or any combination thereof. In some embodiments, binding of a target peptide by an engineered scaffold protein or a fusion protein thereof described herein is capable of modulating the bioavailability of a therapeutic partner described herein. For example, binding of a target peptide by an engineered scaffold protein or a fusion protein thereof to a therapeutic partner (e.g., a target peptide) can protect the therapeutic partner from degradation and enable slow release of the therapeutic partner from binding to the engineered scaffold protein over a certain period of time, extending the intracellular or extracellular half-life of the therapeutic partner or any combination thereof.

In some embodiments, binding of a target peptide by an engineered scaffold protein or a fusion protein thereof described herein can modulate native protein interactions. In some embodiments, binding of a target peptide by an engineered scaffold protein or a fusion protein thereof described herein can enhance or disrupt native protein interactions. Examples of modulating protein interactions include enhancing or disrupting receptor-ligand interactions resulting in modulation of corresponding cell signaling pathway(s). In some embodiments, binding of a target peptide by an engineered scaffold protein or a fusion protein thereof described herein can activate a binding site. In some embodiments, activation of a binding site comprises activation of a signaling pathway. In some embodiments, the signaling pathway that can be activated by an engineered scaffold protein or a fusion protein thereof can include GPCRs, Ion-Channels, Protein Pumps, Enzyme-linked receptors, Single-pass membrane proteins, or secreted proteins. Examples of signaling pathways that can be activated by an engineered scaffold protein described herein include MAPK-PK, RAS/RAF, RHO, FAK1, MEK/MAPK, MAK, MKK, AKT, PI3K-AKT, EGF receptor, Her2 receptor, Her3 receptor, Her4 receptor, p38, NF-Kβ, NGF, NT-3, NT-4, BDNF, JNK, neurotrophin, PLC-71, estrogen receptors, progesterone receptors, androgen receptors, GPER30, PIK3/PTEN, VEGF receptor pathway inhibitors, cell adhesion, TGFbeta/SMAD, WNT, Hedgehog/GLI, HIF1 alpha, JAK/STAT, Notch, CD95/Apo1L, mesolimbic dopamine, mesocortical dopamine, nigrostriatal dopamine, nigrostriatal dopamine, serotonergic pathways, acetylcholine, GABAnergic, glutamatergic, control of Gl/S transition, DNA damage control, and apoptosis. In some embodiments, the signaling pathways include other signaling pathways as disclosed herein. Accordingly, in some embodiments provided herein is a method of activation of a signaling pathway comprising the use of an engineered scaffold described herein.

In some embodiments, binding of a target peptide by an engineered scaffold protein or a fusion protein thereof described herein can inhibit or occlude a binding site. In some embodiments, inhibition or occlusion of the binding site comprises disruption of the protein binding to one or more binding partners. In some embodiments, the engineered scaffold protein or the fusion protein thereof described herein can form an intramolecular interaction with the target peptide. In some embodiments, the engineered scaffold protein or the fusion protein thereof described herein can form an intermolecular interaction with the target peptide. In some embodiments, the intramolecular and/or intermolecular interactions with the target peptide results in blocking a protein comprising the target peptide. In some embodiments, the intramolecular and/or intermolecular interactions can sterically occlude the target protein binding site. In some embodiments, the engineered scaffold protein or the fusion protein thereof may disrupt the protein binding activity by blocking of active site, hydrophobic pocket, or causing steric hinderance on the surface of the target. In some embodiments, binding of the target peptide on the protein by the engineered scaffold protein or the fusion protein thereof described herein can partially or fully blocks, occludes, inhibits, or neutralizes a biological activity of the protein. In some embodiments, binding of the target peptide on the protein by the engineered scaffold protein or the fusion protein thereof described herein can reduce access to the protein's binding site. In some embodiments, binding of the target peptide on the protein by the engineered scaffold protein or the fusion protein thereof described herein can block access to the protein's binding site. In some embodiments, binding of the target peptide on the protein by the engineered scaffold protein or the fusion protein thereof described herein can inhibit with signaling of the protein. In some embodiments, binding of the target peptide on the protein by the engineered scaffold protein or the fusion protein thereof described herein can block signaling pathways of the protein. In some embodiments, binding of the target peptide on the protein by the engineered scaffold protein or the fusion protein thereof described herein can inhibit binding of signaling molecules. Accordingly, in some embodiments provided herein is a method of occluding the binding of a protein to one or more binding partners or inhibiting a biological activity of the protein comprising the use of an engineered scaffold described herein. A schematic model of occlusion by an engineered scaffold protein described herein or a fusion protein thereof can be seen in FIG. 33.

In some embodiments, binding of a target peptide by an engineered scaffold protein or a fusion protein thereof described herein can induce conformational biasing. In some embodiments, induction of conformational biasing comprises stabilizing or locking a protein comprising the target peptide in a conformational state. In some embodiments, the conformation state is an active conformational state. In some embodiments, the conformational state is a partially active conformational state. In some embodiments, the conformational state is an inactive conformational state. In some embodiments, the engineered scaffold protein or the fusion protein thereof described herein selectively binds to the target peptide comprised in the protein, the binding of which may not affect the function of the protein comprising the target peptide. Accordingly, in some embodiments, use of the engineered scaffold protein or the fusion protein thereof described herein can stabilize a protein comprising the target peptide in a functional conformational state. In some embodiments, the functional conformation state is an active, a basal, a partially active, or an inactive conformational state. In some embodiments, the binding of the target peptide by the engineered scaffold protein or the fusion protein thereof described herein can correct misfolded proteins. In some embodiments, the binding of the target peptide by the engineered scaffold protein or the fusion protein thereof described herein can correct misfolded proteins. In some embodiments, correction of misfolded proteins can restore protein biological activity or function. In some embodiments, the binding of the target peptide by the engineered scaffold protein or the fusion protein thereof described herein can prevent protein dissociation. In some embodiments, the binding of the target peptide by the engineered scaffold protein or the fusion protein thereof described herein can prevent degradation of one or more proteins. Accordingly, in some embodiments provided herein is a method of stabilizing or locking a protein comprising a target peptide comprising the use of an engineered scaffold described herein.

In some embodiments, binding of a target peptide by an engineered scaffold protein or a fusion protein thereof described herein can prevent nuclear localization. In some embodiments, prevention of nuclear localization comprises blocking the transportation of a protein comprising the target peptide into the nucleus. In some embodiments, the binding of the target peptide by the engineered scaffold protein or the fusion protein thereof described herein can block a nuclear localization signal of the protein. In some embodiments, the binding of the target peptide by the engineered scaffold protein or the fusion protein thereof described herein can block a nuclear export signal of a protein. In some embodiments, prevention of nuclear localization by binding of the target peptide by the engineered protein described herein can occur in the presence of a nuclear localization signal. In some embodiments, binding of the target peptide by the engineered scaffold protein or the fusion protein thereof described herein can prevent the protein from one or more protein-protein interactions. In some embodiments, the protein-protein interactions can result in localization to the nucleus in the form of a complex. Thus, in some embodiments, the binding of the target peptide by the engineered protein or the fusion protein thereof described herein can prevent the protein from forming the complex and translocating to the nucleus. Accordingly, in some embodiments provided herein is a method of preventing nuclear localization comprising the use of an engineered scaffold protein or a fusion protein thereof described herein.

In some embodiments, binding of a target peptide by an engineered scaffold protein or a fusion protein thereof described herein can disrupt membrane binding. In some embodiments, disruption of membrane binding comprises reducing or preventing an attachment or insertion of a protein comprising the target peptide into the cell membrane. In some embodiments, the attachment can be prevented by blocking access to the protein's hydrophobic residues. In some embodiments, disruption of membrane binding can comprise an increase in soluble protein. In some embodiments, disruption of membrane binding can lead to aberrant targeting of the protein to a cell's cytoplasm and/or intracellular membrane in vitro. In some embodiments, disruption of membrane binding can lead to aberrant targeting of the protein to a cell's cytoplasm and/or intracellular membrane in vivo. In some embodiments, aberrant targeting of the protein can result in impaired access to pathways. In some embodiments, impaired access to pathways can prevent membrane binding partner molecules from signalling normally. Accordingly, in some embodiments provided herein is a method of disrupting membrane binding comprising the use of an engineered scaffold protein or a fusion protein thereof described herein.

In some embodiments, binding of a target peptide by an engineered scaffold protein or a fusion protein thereof described herein can modulate macromolecule interactions. In some embodiments, the macromolecule interactions comprise multivalent binding, dynamic binding, and coupled folding and binding interactions. In some embodiments, modulation of macromolecule interactions can include any suitable macromolecule. Non-limiting examples of macromolecules include disaccharides, polysaccharides, proteins, lipids, fatty acids, nucleic acids, crosslinked or non-crosslinked polymers, organometallic compounds, and other macromolecular scaffolds. In some embodiments, the macromolecule comprises the aggregation of two or more macromolecules. In some embodiments, the macromolecule interactions comprise protein-protein interactions, protein-DNA interactions, and protein-RNA interactions. In some embodiments, modulation of macromolecule involves a macromolecule binding to a protein. In some embodiments, the macromolecule that binds to the protein can be a protein binding partner, an antigen, an epitope, a peptide, a ligand, a receptor, a carbohydrate, a chemical, a small molecule, or an inhibitor. In some embodiments, binding of the target peptide by the engineered scaffold protein or the fusion protein thereof can result in increased protein binding to macromolecule. In some embodiments, binding of the target peptide by the engineered scaffold protein or the fusion protein thereof can result in decreased protein binding to macromolecule. In some embodiments, the binding of the target peptide by the engineered scaffold protein or the fusion protein thereof can be used to inhibit one or more macromolecule from binding the protein. In some embodiments, the binding of the target peptide by the engineered scaffold protein or the fusion protein thereof increases binding, reduces binding, or changing the conditions under which the protein binds to a macromolecule. Accordingly, in some embodiments provided herein is a method of modulating macromolecule interactions comprising the use of an engineered scaffold protein or a fusion protein thereof described herein.

In some embodiments, binding of a target peptide by an engineered scaffold protein or a fusion protein thereof described herein can occlude or sequester post-translational modifications (PTMs). In some embodiments, occlusion or sequestration of PTMs can involve one or more PTMs. Examples of PTMs can include phosphorylation, acetylation, methylation, N-linked glycosylation with different sugars, O-linked glycosylation with different sugars, citrullination or deimination, crotonylation, butyrylation, ubiquitination, C-mannosylation, methionine oxidation, sulfation, amidation, sumoylation, S-nitrosylation or nitrosylation, neddylation, deimination, OclcNAc, ADP-ribosylation, fattenylation, ufmylation, prenylation, myristoylation, S-palmitoylation, formylation, carboxylation, hydroxylation, and Proline cis-trans isomerization. In some embodiments, the binding of the target peptide by the engineered scaffold protein or the fusion protein thereof can be used to partially inhibit access to PTMs on a protein. In some embodiments, the protein can include, but is not limited to, chaperone proteins, effector proteins, assembler proteins, scavenger proteins, degradation proteins, or enzymes. In some embodiments, the binding of the target peptide by the engineered scaffold protein or the fusion protein thereof can be used to inhibit access to PTMs on the protein. In some embodiments, the binding of the target peptide by the engineered scaffold protein or the fusion protein thereof can be used to block and/or sequester PTMs from effector proteins. In some embodiments, the binding of the target peptide by the engineered scaffold protein or the fusion protein thereof can be used to block PTMs from enzymes that catalyze the removal of PTMs. In some embodiments, blocking PTMs can decrease the functional state in which the protein can exist in the cell. In some embodiments, blocking PTMs can decrease the activity of the protein. Accordingly, in some embodiments provided herein is a method of occluding or sequestering PTMs comprising the use of an engineered scaffold protein or a fusion protein thereof described herein.

In some embodiments, binding of a target peptide by an engineered scaffold protein or a fusion protein thereof described herein can modulate PTMs. Accordingly in some embodiments, modulation of PTMs comprises a method of stimulating the activity of an endogenous or exogenous PTM enzyme capable of producing and/or removing PTMs. In some embodiments, the endogeneous or exogenous PTM enzyme can result in a PTM alteration. In some embodiments, the PTM alteration comprises phosphorylation, acetylation, methylation, N-linked glycosylation with different sugars, O-linked glycosylation with different sugars, citrullination or deimination, crotonylation, butyrylation, ubiquitination, C-mannosylation, methionine oxidation, sulfation, amidation, sumoylation, S-nitrosylation or nitrosylation, neddylation, deimination, OclcNAc, ADP-ribosylation, fattenylation, ufmylation, prenylation, myristoylation, S-palmitoylation, formylation, carboxylation, hydroxylation, proline cis-trans isomerizationdeubiquitination (DUB), dephosphorylation, deglycosylation, desumoylation, deacetylation, de-S-nitrosylation or denitrosylation, decitrullination or dedeimination, deneddylation, removal of OclcNAc, de-ADP-ribosylation, demethylation, de-hydroxylation, defattenylation, deufmylation, deprenylation, demyristoylation, de-S-palmitoylation, tyrosine desulfation, deformylation, decarboxylation, deamidation, and any combination thereof. Examples of the endogenous or exogenous PTM enzymes include carboxylate-amine ligases, cyclases, dehydrogenases, cyclodehydratase decarboxylases, epimerases, hydroxylases, peptidases, dehydratases, transferases, esterases, oxygenases and isomerases, ubiquitin ligase, SUMO transferase, methyltransferase, demethylase, acetyltransferase, glycosyltransferase, palmitoyltransferase and/or related hydrolase, lanthionine bond forming enzymes, cytolysin forming enzymes, cyanobactin forming enzymes, thiopeptide forming enzymes, conopeptide forming enzymes, microviridin forming enzymes, cyclotide forming enzymes, bacteriocin forming enzymes and subtilosin forming enzymes. In some embodiments, binding of a target peptide by an engineered scaffold protein or a fusion protein thereof increased a biological activity of an endogenous or exogenous PTM enzyme. In some embodiments, binding of a target peptide by an engineered scaffold protein or a fusion protein thereof decreased the biological activity of an endogenous or exogenous PTM enzyme. Accordingly, in some embodiments provided herein is a method of modulating PTMs comprising the use of an engineered scaffold protein or a fusion protein thereof described herein.

In some embodiments, binding of a target peptide by an engineered scaffold protein or a fusion protein thereof described herein can induce de-phosphorylation. In some embodiments, induction of de-phosphorylation comprises increasing enzymatic activity of phosphatase enzymes. In some embodiments, the engineered scaffold protein or the fusion protein thereof increases the binding of phosphatase to a target protein comprising the target peptide. Examples of phosphatase enzymes include, but is not limited to, bacterial alkaline phosphatases, mammalian alkaline phosphatases, plant acid phosphatases, mammalian acid phosphatases and alkaline phosphatase conjugates. Accordingly, in some embodiments provided herein is a method of inducing de-phosphorylation comprising the use of an engineered scaffold protein or a fusion protein thereof described herein.

In some embodiments, binding of a target peptide by an engineered scaffold protein or a fusion protein thereof described herein can induce protein isomerization. In some embodiments, induction of protein isomerization involves the activation of an isomerase enzyme. In some embodiments, activation of the isomerase enzyme results in increase racemization of a target protein. In some embodiments, activation of the isomerase enzyme results in increase epimerization of a target protein. In some embodiments, the isomerase enzyme can be aldose atriose phosphate isomerase, bisphosphoglycerate mutase, triosephosphate isomerase, phosphomutases, epimerases, racemases, and carbon-skeleton mutases, or photoisomerase. In some embodiments, the engineered scaffold protein or the fusion protein thereof described herein can induce isomerization of an amino acid residue. In some embodiments, the amino acid residue is proline. In some embodiments, binding of the target peptide by the engineered scaffold protein or a fusion protein thereof described herein can increase the isomerization of proline. In some embodiments, the isomerization of proline is catalyzed by a proline isomerase. In some embodiments, binding of the target peptide by an engineered scaffold protein or the fusion protein thereof described herein increases the biological activity of the proline isomerase resulting in one or more conformations of proline. In some embodiments, the one or more conformations of proline include cis conformation and trans conformation. In some embodiments, binding of the target peptide by an engineered scaffold protein or the fusion protein thereof described herein increases cis-trans isomerization of proline. Accordingly, in some embodiments provided herein is a method of inducing protein isomerization comprising the use of an engineered scaffold protein or a fusion protein thereof described herein.

In some embodiments, binding of a target peptide by an engineered scaffold protein or a fusion protein thereof described herein can modulate ATPase/GTPase activity. In some embodiments, modulation of ATPase/GTPase activity comprises activating the ATPase/GTPase. In some embodiments, modulation of ATPase/GTPase activity comprises inhibiting the ATPase/GTPase. In some embodiments, binding of the target peptide by an engineered scaffold protein or the fusion protein thereof described herein increases ATP hydrolysis. In some embodiments, binding of the target peptide by an engineered scaffold protein or the fusion protein thereof described herein increases GTP hydrolysis. In some embodiments, modulation of ATPase activity can be used to treat a disease. In some embodiments, the disease comprises aberrant ATPase activity. In some embodiments, modulation of GTPase activity can be used to treat a disease. In some embodiments, the disease comprises aberrant GTPase activity. Examples of a disease with aberrant ATPase/GTPase activity include, but are not limited to, stroke, cardiovascular diseases (e.g., angina pectoris, myocardial infarction, chronic ischemic heart disease, hypertensive heart disease, pulmonary heart disease, valvular heart disease, rheumatic fever, rheumatic heart disease, endocarditis, mitral valve prolapse, aortic valve stenosis, congenital heart disease, valvular and vascular obstructive lesions, atrial or ventricular septal defect, patent ductus arteriosus, and myocardial disease), Wilson disease, Menkes syndrome, kidney disorders (e.g., acute and chronic glomerulonephritis, rapidly progressive glomerulonephritis, nephrotic syndrome, focal proliferative glomerulonephritis, systemic lupus erythematosus, Goodpasture's syndrome, multiple myeloma, diabetes, neoplasia, sickle cell disease, chronic inflammatory diseases, acute tubular necrosis, acute renal failure, polycystic renal diseasemedullary sponge kidney, medullary cystic disease, nephrogenic diabetes, renal tubular acidosis, tubulointerstitial diseases, acute and rapidly progressive renal failure, chronic renal failure, and nephrolithiasis), hypotension, hypertension, ischemic injury, neurological disorders (e.g., Alzheimer's disease and Parkinson's disease), muscle disorders, pulmonary disorders (e.g., emphysema, chronic bronchitis, bronchial asthma, bronchiectasis, sarcoidosis, pneumoconiosis, hypersensitivity pneumonitis, Goodpasture's syndrome, idiopathic pulmonary hemosiderosis, pulmonary alveolar proteinosis, desquamative interstitial pneumonitis, chronic interstitial pneumonia, fibrosing alveolitis, hamman-rich syndrome, pulmonary eosinophilia, diffuse interstitial fibrosis, Wegener's granulomatosis, lymphomatoid granulomatosis, and lipid pneumonia), hepatic disorders (e.g., hepatic vein thrombosis, portal vein obstruction, thrombosis, hepatitis, and cirrhosis), proliferative disorders (e.g., neoplasms or tumors such as carcinomas, sarcomas, adenomas, and myeloid leukemia), and disorders in which a positive ionotropic effect is desired. Accordingly, in some embodiments provided herein is a method of modulating ATPase/GTPase activity comprising the use of an engineered scaffold protein or a fusion protein thereof described herein.

In some embodiments, binding of a target peptide by an engineered scaffold protein or a fusion protein thereof described herein can prevent pro-protein processing. In some embodiments, prevention of pro-protein processing comprises blocking proteolytic cleavage. In some embodiments, blocking proteolytic cleavage involves binding, partial binding, blocking, or burying the catalytic site from one or more proteases. In some embodiments, the one or more proteases can include proprotein convertases, serine proteases, metalloproteases, cysteine proteases, threonine proteases and aspartic acid. In some embodiments, prevention of pro-protein processing can result in a reduction in the number of bioactive peptides. In some embodiments, prevention of pro-protein processing can result in a reduction in the number of bioactive proteins. In some embodiments, prevention of pro-protein processing can result in a reduction in the number of active enzymes. Accordingly, in some embodiments provided herein is a method of preventing pro-protein processing comprising the use of an engineered scaffold protein or a fusion protein thereof described herein.

In some embodiments, binding of a target peptide by an engineered scaffold protein or a fusion protein thereof described herein can induce protein disaggregation and/or refolding. In some embodiments, induction of protein disaggregation and/or refolding involves the activation of chaperone enzymes. Examples of chaperone enzymes can be Hsp90 family of proteins, Hsp70 family of proteins, immunophilin family of proteins, peptidase C56 family of proteins, Pplase family of proteins, 14-3-3 family of proteins, small heat-shock protein group of proteins, small GTPase family of proteins, Hsp40 (DnaJ) family of proteins, and clusterin, Grp170, calreticulin, Hsp105, Hsp70-interacting protein (CHIP), alpha-crystallin or any of the combinations thereof. In some aspects, the activation of chaperone enzymes results in an increase in the number of refolded proteins. In some aspects, the activation of chaperone enzymes results in an increase in the number of disaggregated proteins. In some embodiments, binding of the target peptide by the engineered scaffold protein or the fusion protein thereof described herein can increase the refolding of a multimeric protein. In some embodiments, the multimeric protein includes, but is not limited to, dimers, trimers, tetramers, pentamers, hexamers, septamers, octamers, or nonamer. The multimeric protein subunits can be identical (homodimer, etc.) or one or more can differ within the native protein (heterodimers, etc.). Accordingly, in some embodiments provided herein is a method inducing protein disaggregation and/or refolding comprising the use of an engineered scaffold protein or a fusion protein thereof described herein.

In some embodiments, binding of a target peptide by an engineered scaffold protein or a fusion protein thereof described herein can induce proteolytic cleavage. In some embodiments, induction of proteolytic cleavage comprises increasing proteolytic cleavage. In some embodiments, increasing proteolytic cleavage comprises an increase in accessibility to a protein to a catalytic enzyme. In some embodiments, the catalytic enzyme is a protease. In some embodiments, increasing proteolytic cleavage involves binding, partial binding, stabilizing, or exposing a catalytic site in the protein to one or more proteases. In some embodiments, the one or more proteases can include proprotein convertases, serine proteases, metalloproteases, cysteine proteases, threonine proteases and aspartic acid. In some embodiments, induction of proteolytic cleavage can result in an increase in the number of bioactive peptides. In some embodiments, induction of proteolytic cleavage can result in an increase in the number of bioactive proteins. In some embodiments, induction of proteolytic cleavage can result in an increase in the number of active enzymes. Accordingly, in some embodiments provided herein is a method of inducing proteolytic cleavage comprising the use of an engineered scaffold protein or a fusion protein thereof described herein.

In some embodiments, binding of a target peptide by an engineered scaffold protein or a fusion protein thereof described herein can induce cell degradation. In some embodiments, induction of cell degradation can induce cell death. In some embodiments, induction of cell degradation and/or cell death comprises activating apoptotic pathways, autophagic cell death pathways, necrotic pathways, complement dependent cytotoxicity, or antibody-dependent cellular cytotoxicity. In some embodiments, the therapeutic methods as disclosed herein can comprises a pharmacological drug conjugated to the engineered scaffold or fusion protein thereof and delivered to a specific cell as a therapeutic. In some embodiments, binding of the target peptide by the engineered scaffold protein or the fusion protein thereof described herein can be used in a therapeutic method wherein the induction of apoptosis is therapeutically desirable. In some embodiments, the therapeutic method comprises increase in apoptosis. In some embodiments, the increase in apoptosis can be used to selective kill a desired target cell. In some embodiments, the binding of the target peptide by the engineered scaffold protein or the fusion protein thereof described herein can induce greater apoptosis in the desired target cell than a non-target cell. In some embodiments, binding of the target peptide by the engineered scaffold protein or the fusion protein thereof described herein can be used in a therapeutic method wherein the induction of autophagy is therapeutically desirable. In some embodiments, the therapeutic method comprises increase in autophagy. In some embodiments, the increase in autophagy can be used to selective kill a desired target cell. In some embodiments, the binding of the target peptide by the engineered scaffold protein or the fusion protein thereof described herein can induce greater autophagy in the desired target cell than a non-target cell. In some embodiments, binding of the target peptide by the engineered scaffold protein or the fusion protein thereof described herein can be used in a therapeutic method wherein the induction of necrosis is therapeutically desirable. In some embodiments, the therapeutic method comprises increase in necrosis. In some embodiments, the increase in necrosis can be used to selective kill a desired target cell. In some embodiments, the binding of the target peptide by the engineered scaffold protein or the fusion protein thereof described herein can induce greater necrosis in the desired target cell than a non-target cell. In some embodiments, binding of the target peptide by the engineered scaffold protein or the fusion protein thereof described herein can be used in a therapeutic method wherein the induction of complement dependent cytotoxicity is therapeutically desirable. In some embodiments, the therapeutic method comprises increase in complement dependent cytotoxicity. In some embodiments, the increase in complement dependent cytotoxicity can be used to selective kill a desired target cell. In some embodiments, the binding of the target peptide by the engineered scaffold protein or the fusion protein thereof described herein can induce greater complement dependent cytotoxicity in the desired target cell than a non-target cell. In some embodiments, binding of the target peptide by the engineered scaffold protein or the fusion protein thereof described herein can be used in a therapeutic method wherein the induction of antibody-dependent cellular cytotoxicity is therapeutically desirable. In some embodiments, the therapeutic method comprises increase in antibody-dependent cellular cytotoxicity. In some embodiments, the increase in antibody-dependent cellular cytotoxicity can be used to selective kill a desired target cell. In some embodiments, the binding of the target peptide by the engineered scaffold protein or the fusion protein thereof described herein can induce greater antibody-dependent cellular cytotoxicity in the desired target cell than a non-target cell. In some embodiments, the therapeutic methods as described herein can be used to treat a disease. In some embodiments, the disease includes, but is not limited to, cancer, infectious disease, neurodegenerative disease, cardiovascular disease, pulmonary disease, muscular disease, hepatic disease, autoimmune and/or inflammatory diseases, and proteinopathies. In some embodiments, the therapeutic methods described herein can be used to selectively kill cancer cells that are resistant to other therapies. Accordingly, in some embodiments provided herein is a method of inducing cell degradation comprising the use of an engineered scaffold protein or a fusion protein thereof described herein.

In some embodiments, binding of a target peptide by an engineered scaffold protein or a fusion protein thereof described herein can block substrate recruitment. In some embodiments, blocking of substrate recruitment inhibits a protein-protein interaction. In some embodiments, binding of the target peptide by the engineered scaffold protein or the fusion protein thereof described herein prevents protein-substrate binding. In some embodiments, binding of the target peptide by the engineered scaffold protein or the fusion protein thereof described herein can restrict active site access. In some embodiments, binding of the target peptide by the engineered scaffold protein or the fusion protein thereof described herein can block essential substrate recruitment sites. In some embodiments, binding of the target peptide by the engineered scaffold protein or the fusion protein thereof described herein can block catalytic sites. In some embodiments, the engineered scaffold protein or the fusion protein thereof can physically block substrate binding. Accordingly, in some embodiments provided herein is a method of blocking substrate recruitment comprising the use of an engineered scaffold protein or a fusion protein thereof described herein.

In some embodiments, binding of a target peptide by an engineered scaffold protein or a fusion protein thereof described herein can induce an immune response. In some embodiments, binding of the target peptide by the engineered scaffold protein or the fusion protein thereof described herein can stimulate an immune response in vivo. In some embodiments, stimulating the immune response results in the upregulation of a gene that encodes a cytokine, chemokine or lymphokine. Examples of such genes include alpha-interferon, gamma-interferon, platelet derived growth factor (PDGF), TNFa, TNFP, GM-CSF, epidermal growth factor (EGF), IL-1, IL-2, IL-4, IL-5, IL-6, IL-10, IL 0 12, IL-18, MHC, CD80, CD86 and IL-15. In some embodiments, binding of the target peptide by the engineered scaffold protein or the fusion protein thereof described herein can be used in the form of a vaccine. In some embodiments, the vaccine comprises inactivated vaccines, live-attenuated vaccines, messenger RNA (mRNA) vaccines, subunit, recombinant, polysaccharide, and conjugate vaccines, neoepitope vaccine, neurodegeneration vaccine, infectious disease vaccine, toxoid vaccines or viral vector vaccines. Accordingly, the vaccines can be provided to induce a therapeutic or prophylactic immune response. In some embodiments, the means to deliver the immunogen is a DNA vaccine, a recombinant vaccine, a protein subunit vaccine, a composition comprising the immunogen, a live-attenuated vaccine or a killed vaccine. Accordingly, in some embodiments provided herein is a method of inducing an immune response comprising the use of an engineered scaffold protein or a fusion protein thereof described herein.

### Therapeutic Uses

Accordingly, the engineered scaffold proteins or fusion proteins thereof disclosed herein may be developed as therapeutic molecules. In some embodiments, the therapeutic molecule can bind to any protein on the cell surface, a secreted protein in the extracellular space, or a protein in the intracellular compartment. The protein target of the therapeutic molecule may be a receptor, cell surface marker, a membrane-bound protein, an enzyme, intracellular protein, a signaling pathway component, or other proteins. The therapeutic molecule may act as an inhibitor where the therapeutic molecule binds to the target protein and inhibits a critical biological activity. It can also act to modulate protein-protein interactions either as an antagonist or agonist. The therapeutic molecule may also modulate the enzymatic activity of the target protein or another protein that interacts with said target protein. For example, by fusing a protease enzyme with the engineered scaffold, the therapeutic molecule may be engineered to bind specifically to a target protein and site cleave the target protein. In some embodiments, the modality prevents the aggregation of proteins by binding to the target protein and thereby changing its biophysical properties. As an example, α-Synuclein is a precursor for plaque formation in many neurodegenerative diseases. The plaques form due to aggregation of α-Synuclein; by binding to the target protein and preventing its aggregation, it may be possible to treat neurodegenerative diseases. Similar to an antibody-drug conjugate, a pharmacological drug may be conjugated to the engineered scaffold or fusion protein thereof and delivered to a specific cell as a therapeutic.

Also provided herein are compositions, systems and methods comprising an engineered scaffold protein or a fusion protein thereof described herein and a therapeutic partner. In some embodiments, the engineered scaffold protein or the fusion protein thereof described herein can be used to deliver the therapeutic partner to a target. In some embodiments, the engineered scaffold protein or fusion protein thereof described herein can serve as a vehicle for delivering the therapeutic partner to the target. In some embodiments, the therapeutic partner is a therapeutic peptide. In some embodiments, the therapeutic peptide can be loaded on the engineered scaffold protein or fusion protein thereof described herein. In some embodiments, the engineered scaffold protein or fusion protein thereof described herein can form a covalent bond to the therapeutic peptide. In some embodiments, the covalent bond is a disulfide bond between a cysteine residue on the engineered scaffold protein or fusion protein thereof described herein and a cysteine residue on the therapeutic peptide. In some embodiments, the therapeutic peptide can be loaded onto part of an amino acid chain of the engineered scaffold protein or the fusion protein thereof described herein. In some embodiments, the amino acid chain of the engineered scaffold protein or fusion protein thereof described herein can include a N-terminal peptide, a C-terminal peptide, or germinating from an intermediary loop on the engineered scaffold protein or the fusion protein thereof described herein. In some embodiments, the engineered scaffold protein or the fusion protein thereof can be linked to the therapeutic peptide at the N-terminus of the engineered scaffold protein or the fusion protein thereof described herein. In some embodiments, the engineered scaffold protein or the fusion protein thereof described herein can be linked to the therapeutic peptide with a N-terminal linker. In some embodiments, the engineered scaffold protein or the fusion protein thereof described herein can be linked to the therapeutic peptide at the C-terminus of the engineered scaffold protein or the fusion protein thereof. In some embodiments, the engineered scaffold protein or the fusion protein thereof described herein can be linked to the therapeutic peptide with a C-terminal linker. In some embodiments, the engineered scaffold protein or the fusion protein thereof described herein can be linked to the therapeutic peptide at any residue on the engineered scaffold protein or the fusion protein thereof. In some embodiments, the engineered scaffold protein or the fusion protein thereof described herein can form a non-covalent bond to the peptide drug. In some embodiments, the non-covalent bond can include, but is not limited to, van der Waal forces, salt bridges or hydrogen bonds. In some embodiments, the engineered scaffold protein or fusion protein thereof described herein and the peptide drug are linked by a linker. In some embodiments, the linker is a proteolytically cleavable linker. Accordingly, an engineered scaffold protein or fusion protein thereof can serve as a vehicle for a therapeutic peptide.

Binding of an engineered scaffold protein or a fusion protein thereof described herein to a therapeutic peptide can serve multiple functions including, but not limited to, cell specificity, tissue specificity, and half-life extension. In some embodiments, the engineered scaffold protein or the fusion protein thereof described herein can be engineered to bind to a specific target. In some embodiments, the specific target is over-expressed on an intended cell type. In some embodiments, the intended cell type is a diseased cell. In some embodiments, the engineered scaffold protein or the fusion protein thereof described herein can selectively bind to the diseased cell over a normal cell. Accordingly, in some embodiments, the engineered scaffold protein or the fusion protein thereof described herein can deliver the therapeutic peptide specifically to the intended cell type. In some embodiments, the therapeutic peptide can be ensconced within the interface of the engineered scaffold protein or the fusion protein thereof described herein. In some embodiments, the engineered scaffold protein or the fusion protein thereof described herein can extend the half-life of the therapeutic peptide. In some embodiments, the engineered scaffold protein or the fusion protein thereof described herein can increase stability of the therapeutic peptide. In some embodiments, the engineered scaffold protein or the fusion protein thereof can protect the therapeutic peptide from proteolytic enzymes. Accordingly, in some embodiments, an engineered scaffold protein or a fusion protein thereof can be used increase target specificity and half-life of a therapeutic peptide.

Binding of an engineered scaffold protein or a fusion protein thereof described herein to a therapeutic peptide can be used for a targeted release of the therapeutic peptide. In some embodiments, the targeted release of the therapeutic peptide can include endocytosis, linker cleavage, release from binding interface, restrained relief, etc. In some embodiments, the therapeutic peptide can be released from the engineered scaffold protein or the fusion protein thereof described herein during the endocytotic process. In some embodiments, the therapeutic peptide can be released from the engineered scaffold protein or the fusion protein thereof described herein by linker cleavage of a specific linker under one or more cellular environmental conditions. In some embodiments, the specific linker can include a protease-sensitive peptide linker, an acid sensitive hydrazone linker, a pH-sensitive linker, or a glutathione-sensitive disulfide linker. In some embodiments, binding of the engineered scaffold protein or the fusion protein thereof to a target cell can induce a conformational change releasing the therapeutic peptide from the interface of the engineered scaffold protein or the fusion protein thereof described herein. In some embodiments, the therapeutic peptide can bind to a therapeutic target while being attached to the engineered scaffold protein or the fusion protein thereof described herein through a long linker. In some embodiments, the long linker can include a long peptide linker or a long non-peptide linker. In some embodiments, a long peptide linker comprises 10 to 50 amino acids in length. In some embodiments, the long peptide linker comprises 10 amino acids, 11 amino acids, 12 amino acids, 13 amino acids, 14 amino acids, 15 amino acids, 16 amino acids, 17 amino acids, 18 amino acids, 19 amino acids, 20 amino acids, 21 amino acids, 22 amino acids, 23 amino acids, 24 amino acids, 25 amino acids, 26 amino acids, 27 amino acids, 28 amino acids, 29 amino acids, 30 amino acids, 31 amino acids, 32 amino acids, 33 amino acids, 34 amino acids, 35 amino acids, 36 amino acids, 37 amino acids, 38 amino acids, 39 amino acids, 40 amino acids, 41 amino acids, 42 amino acids, 43 amino acids, 44 amino acids, 45 amino acids, 46 amino acids, 47 amino acids, 48 amino acids, 49 amino acids, 50 amino acids. Accordingly, an engineered scaffold protein or a fusion protein thereof described herein can be utilized as a targeted drug delivery system for a therapeutic peptide.

Also provided herein methods of treatment comprising the engineered scaffold protein or the fusion protein thereof described herein. In some embodiments, the engineered scaffold protein or the fusion protein thereof can enhance a therapeutic treatment for a disease in a subject. In some embodiments, the therapeutic treatment can include chimeric antigen receptor (CAR) T-cell therapy and lysosomal targeted chimera (LYTAC) therapy. CAR-T cell therapy is a type of cancer immunotherapy treatment that uses immune cells called T cells that are genetically altered to enable them in locating in destroying cancer cells more effectively. In some embodiments, the engineered scaffold protein or the fusion protein thereof described herein can replace a binding domain of a chimeric antigen receptor (CAR). In some embodiments, the binding domain of the CAR is the scFV domain. In some embodiments, the engineered scaffold protein or the fusion protein thereof described herein can be hinged to the endogenous transmembrane domain of the CAR. In some embodiments, the engineered scaffold protein or the fusion protein thereof described herein can be linked to the endogenous transmembrane domain of the CAR. In some embodiments, the engineered scaffold protein or the fusion protein thereof described herein linked to the CAR can improve stability, half-life and specificity of the CAR. Accordingly, the engineered scaffold protein or the fusion protein thereof can be used to enhance CAR T-cell therapy. Lysosomal targeted chimeras (LYTAC) are known in the art to induce the degradation of extracellular and membrane proteins via the endosome-lysosome pathway. In some embodiments, an engineered scaffold protein or a fusion protein thereof described herein can replace an antibody that is fused to a ligand. In some embodiments, the ligand can bind to a receptor. In some embodiments, the receptor can mediate internalization of a cell. In some embodiments, the ligand can be conjugated to the engineered scaffold protein of the fusion protein thereof described herein for use as a LYTAC to degrade targets on the cell membrane. Accordingly, the engineered scaffold protein or the fusion protein thereof described herein linked to the LYTAC can improve stability, half-life and specificity of the LYTAC.

Accordingly, disclosed herein are methods of prophylaxis/treatment of a disease or disorder associated with a target peptide. Further disclosed herein are methods of treatment comprising administering to a subject in need thereof an effective amount of an engineered scaffold protein, a fusion protein thereof, or a pharmaceutical composition comprising the same. A subject in need thereof can be a subject who is suffering from or is at a risk of developing a disease or disorder associated with a target peptide.

In some embodiments, methods, systems, and compositions comprising an engineered scaffold protein or fusion protein thereof for use as a treatment, pharmaceutical, therapy, or prophylaxis, also comprise a molecular transporter. Exemplary, non-limiting molecular transporters are described en supra. In some embodiments, a suitable molecular transporter can be selected for use in a treatment, pharmaceutical, therapy, or prophylaxis depending on the desired location for the treatment, pharmaceutical, therapy, or prophylaxis to have a biological or therapeutic activity. For example, a molecular transporter can be selected to penetrate a phospholipid, mitochondrial, endosomal or nuclear membrane; to cross the across the blood-brain, trans-mucosal, hematoretinal, skin, gastrointestinal and/or pulmonary barriers, or to target a cellular destination, e.g. into the cytoplasm (cytosol, endoplasmic reticulum, Golgi apparatus, the ribosome, the mitochondria, a lysosome, or a peroxisome, or etc.) or the nucleus; or any combination thereof.

### Devices and Diagnostics

Engineered scaffold proteins described herein can be used in a variety of detection methods including biochemical, histological, and cell-based methods, system, and compositions among others. Accordingly, provided herein are methods, system, and compositions of detecting one or more target peptides or a fragment thereof, comprising an engineered scaffold protein or a fusion protein thereof described herein. In some embodiments, methods, system, and compositions of detecting one or more target peptides or a fragment thereof, comprise an engineered scaffold protein or a fusion protein thereof and a detectable moiety. In some embodiments, the detectable moiety indicates the presence of the target peptide upon contact of engineered scaffold protein to the target peptide or fragment thereof, emitting or producing a detectable signal.

In some embodiments, methods and systems of detecting one or more target peptides or a fragment thereof comprises obtaining a sample from a subject (e.g., a biological sample), contacting the sample with one or more engineered scaffold protein, fusion protein thereof, and a detectable moiety or compositions comprising one or more engineered scaffold protein or fusion protein thereof, and a detectable moiety, and detecting the detectable signal emitted from the detectable moiety.

In some embodiments, the engineered scaffold protein may be linked, conjugated (e.g., covalently or non-covalently) or fused to the detectable moiety. In such embodiments, the engineered scaffold protein comprises the detectable moiety. In some embodiments, the detectable moiety comprises a primary moiety. In some embodiments, the primary moiety can emit or produce a detectable signal or act as a substrate to enable the emission or production of a detectable signal upon contact of engineered scaffold protein to the target peptide or fragment thereof. In some embodiments, the primary moiety can bind to or be attached to (e.g, by conjugation, fusion, linkers, etc.) the engineered scaffold protein. In some embodiments, the primary moiety comprises any suitable molecule that can emit or produce a detectable signal or act as a substrate to enable the emission or production of a detectable signal. In some embodiments, the primary moiety comprises peptides (e.g., a tag), nucleic acids, lipids, antibodies, aptamers, modified amino acids, fluorescence labels, nanoparticles, fluorescent proteins, oligos, antibodies, aptamers, and the like. In some embodiments, the detectable moiety comprises a signal amplification moiety which can amplify a detectable signal. In some embodiments, signal amplification moities can be attached to the primary moiety and/or the engineered scaffold protein, or be free floating in a detection reagent or on a substrate. In some embodiments, the detectable moiety comprises a signal amplification moiety which can amplify a detectable signal upon contact with a primary moiety, an engineered scaffold protein, or upon contact of an engineered scaffold protein to a target peptide or a fragment thereof. In some embodiments, signal amplification moities comprises tags, such as fluorescence labels, nanoparticles, fluorescent proteins, oligos, antibodies, aptamers, and the like. An exemplary embodiment of engineered scaffold proteins and detectable moities can be seen in FIG. 36.

In the context of an assay, a target peptide or a fragment thereof can be 1-5 amino acids in length. In some embodiments, assays described herein can detect short linear epitopes at a single molecule level. Such level of detection can be used to analyzed complex samples, including the proteom of a cell or tissue sample. In some embodiments, the target peptide or fragment thereof can be immobilised on a substrate, e.g., glass, beads, or DNA, by direct or indirect conjugation, or can be free floating in a sample. Non-limiting examples of suitable assays comprising an engineered scaffold protein or a fusion thereof include ELISA, western blot, or imaging of specific targets within cells or tissues.

In some embodiments, methods, systems, and compositions of detecting detects more than one target peptide or fragment thereof. In some embodiments, methods, systems, and compositions of detecting detects 2, 3, 4, 5, 6, 7, 8, 9, 10, 11, 12, 13, 14, 15, 16, 17, 18, 19, 20, or more target peptides or fragments thereof in a sample. In some embodiments, wherein methods, systems, and compositions of detecting detects 2, 3, 4, 5, 6, 7, 8, 9, 10, 11, 12, 13, 14, 15, 16, 17, 18, 19, 20, or more target peptides or fragments thereof in a sample, such methods, systems, and compositions of detecting comprises 2, 3, 4, 5, 6, 7, 8, 9, 10, 11, 12, 13, 14, 15, 16, 17, 18, 19, 20, or more uniquely labeled engineered scaffold proteins. Accordingly, in some embodiments provided herein are methods, compositions, and systems of obtaining a fingerprint, profile, signature, or read-counts of a sample.

Also provided herein are compositions, systems and methods for detecting the presence or absence of a bioanalyte comprising the use of a detection agent comprising an engineered scaffold protein or a fusion protein thereof described herein. A bioanalyte as described herein refers to a peptide, small molecule/metabolite (e.g., dNTP), nucleic acid (e.g., double stranded DNA, single stranded DNA, RNA, circular DNA, circular RNA, amino acid, etc.), or a whole cell, cellular compartment/organelle, antibody, serum component, hemoglobin, etc.

Detecting the presence or absence of a bioanalyte with the detection agent comprising the engineered scaffold protein or the fusion protein thereof described herein involves binding and labeling the bioanalyte. For example, labeling the bioanalyte by an engineered scaffold protein or a fusion protein thereof described herein includes a specific tag conjugated to the engineered scaffold protein or the fusion protein thereof described herein. Examples of the specific tag include, an imaging agent, a polypeptide linked to an imaging agent, a chelating agent, a fluorescent label, a luciferase enzyme, a self-labeling protein, a peptide tag (e.g., ALFA-tag, AviTag, FLAG-tag, HA-tag, iCapTag, etc.) and a Biotin Carboxyl Carrier Protein. By labeling the engineered scaffold protein or the fusion protein thereof with the specific tag, the presence or absence of a bioanalyte in a sample can be determined by interaction between the engineered scaffold protein or fusion protein thereof and the bioanalyte. Such interaction can identify the specific bioanalyte that the engineered scaffold or fusion protein interacts with. Similarly, a peptide/protein sequence encoding a post-translational modification (PTM) may be identified by engineering an engineered scaffold or fusion protein to a specific peptide sequence that encodes the PTM. The detection may be for protein sequencing, protein fingerprinting, protein quantification or protein identification. In some embodiments, a detection agent comprising an engineered scaffold protein or a fusion protein thereof described herein can be used to detect a bioanalyte or a biological molecule. In some embodiments, the bioanalyte or the biological molecule includes, but is not limited to, small molecule/metabolite (e.g., dNTP), nucleic acid (e.g., double stranded DNA, single stranded DNA, RNA, circular DNA, circular RNA, amino acid, etc.), glucose, potassium, lactate, uric acid, sodium, chloride, proteins (e.g., receptors, channels, soluble protein, etc.), lipids, and cells. An example of detecting such a bioanalyte with the any of the engineered scaffold proteins disclosed herein is to generate a diverse set of engineered scaffold proteins that bind an orthogonal set of peptide sequences serving as a peptide barcode.

In some embodiments, a detection agent comprising an engineered scaffold protein or a fusion protein thereof described herein is provided in a form of a device. In some embodiments, the device comprises a solid support, and a detection agent holding part. In some embodiments, the detection agent holding part includes a site on the device to which a liquid sample is added. Examples of the liquid sample includes, urine, blood, sweat, mucus, interstitial fluid, chyme, saliva, bile, semen, cerebrospinal fluid, phlegm, pus, breast milk, and synovial fluid. By adding the liquid sample containing the bioanalyte and/or biological molecule to the device, through the detection agent holding part of the device, the detection agent comprising the engineered scaffold protein or the fusion protein thereof described herein can bind to the bioanalyte and/or biological molecule to be measured or quantified. In some embodiments, the device can comprise a solid support such as a porous membrane or microfluidic chip. In some embodiments, the device is a microfluidic chip. Microfluidic chips can be manufactured by methods known in the art, for example, can be manufactured by preparing a flow channel having a mixing section and a reaction section, one or more inlets, and a waste liquid storage section, on a small piece of glass or plastic. The inlet is used for injecting a liquid sample containing the substance to be measured, and an inlet for injecting a washing liquid and/or an enzyme substrate solution into the channel may be provided separately, as necessary. In some embodiments, the device is formed by a microfluidic chip which includes a device having only a bioanalyte and/or biological molecule capturing part. In some embodiments, the bioanalyte and/or biological molecule capturing part comprises an engineered scaffold protein or a fusion protein thereof described herein. In some embodiments, the device can be a kit that comprises a detection agent comprising an engineered scaffold protein or a fusion protein thereof described herein.

In some embodiments, the compositions, systems and methods for detecting the presence or absence of a bioanalyte comprises an assay system in which a kit comprising a detection agent comprising the engineered scaffold protein or the fusion protein described herein and a measurement apparatus capable of measuring a signal generated from a reporter substance of the detection agent. In some embodiments, the detection agent is held in a detection agent holding part. In some embodiments, the detection agent holding part is combined with the measurement apparatus. In some embodiments, the signal generated can include fluorescence, luminescence, color, turbidity, radiation, or signal on a spectrometer. In some embodiments, the spectrometer includes, but is not limited to, an optical absorption spectrometer, an optical emission spectrometer, an electron spectroscopy, a mass spectrometer, a time-of-flight spectrometer, and a magnetic spectrometer. In some embodiments, the measurement apparatus can comprise a liquid pump. In some embodiments, the liquid feed pump can be used to control the speed at which the liquid sample containing the bioanalyte and/or biological molecule moves on the device.

In some embodiments, a device comprising a detection agent includes an engineered scaffold protein or a fusion protein thereof that is immobilized on a solid support in a state where the three-dimensional structure of the engineered scaffold protein or the fusion protein is retained. In some embodiments, the liquid sample is added to the device comprising the immobilized engineered scaffold protein or fusion protein thereof. In some embodiments, the immobilized engineered scaffold protein or fusion protein thereof forms a complex with a bioanalyte or biological molecule in the liquid sample, and a signal is generated upon binding. In some embodiments, the signal generated can be used to create a standard cure or a dose-effect curve. In some embodiments, a signal intensity measured when a liquid sample containing an unknown concentration of a bioanalyte or a biomolecule can be inserted into the standard curve to determine the amount of the bioanalyte or biomolecule. A biological molecule as described herein, refers to a protein (e.g., receptors, channels, soluble protein, membrane bound protein, post-translationally modified proteins), a lipid, a carbohydrate, etc.

In some embodiments, described herein assay method for detecting, monitoring, or measuring a given ligand, such as a target ligand, in a sample which comprises inducing an expression vector described herein to express an engineered scaffold protein or a fusion protein thereof and then contacting the engineered scaffold protein or a fusion protein thereof with the sample and observing whether the engineered scaffold protein or a fusion protein thereof interacts with the target ligand. In such embodiments, methods comprise the use of a polypeptide display system.

Further details and examples can be found in the Examples provided herein.

### Sequences and Tables

TABLE 1
Exemplary Human Protein Scaffolds and Associated Sequences

| PDB | | Protein Name | | |
|---|---|---|---|---|
| Entry ID | PDB Entry Name | (Protein Scaffold) | Protein Sequence | SEQ ID NO: |
| 1cp3 | CRYSTAL | APOPAIN | NSYKMDYPEMGLCIIINNKNFHKSTGMTSRSGTDVDAANLRE | 1 |
| | STRUCTURE OF THE | | TFRNLKYEVRNKNDLTREEIVELMRDVSKEDHSKRSSFVCVL | |
| | COMPLEX OF | | LSHGEEGIIFGTNGPVDLKKITNFFRGDRCRSLTGKPKLFIIQAC | |
| | APOPAIN WITH THE | | RGTELDCGIEHKIPVDADFLYAYSTAPGYYSWRNSKDGSWFI | |
| | TETRAPEPTIDE | | QSLCAMLKQYADKLEFMHILTRVNRKVATEFESFSFDATFHA | |
| | INHIBITOR ACE- | | KKQIPCIVSMLTKELYFYH | |
| | DVAD-FMC (SEQ ID | | | |
| | NO: 311) | | | |
| 1d5x | X-RAY CRYSTAL | HLA CLASS II | GDTRPRFLEQVKHECHFFNGTERVRFLDRYFYHQEEYVRFDS | 2 |
| | STRUCTURE OF HLA- | HISTOCOMPATI | DVGEYRAVTELGRPDAEYWNSQKDLLEQKRAAVDTYCRHN | |
| | DR4 COMPLEXED | BILITY | YGVGESFTVQRRVYPEVTVYPAKNLLVCSVNGFYPGSIEVRW | |
| | WITH DIPEPTIDE | ANTIGEN | FRNGQEEKTGVVSTGLIQNGDWTFQTLVMLETVPRSGEVYTC | |
| | MIMETIC AND SEB | | QVEHPSLTSPLTVEWRA | |
| 1fch | CRYSTAL | PEROXISOMAL | SATYDKGYQFEEENPLRDHPQPFEEGLRRLQEGDLPNAVLLFE | 3 |
| | STRUCTURE OF THE | TARGETING | AAVQQDPKHMEAWQYLGTTQAENEQELLAISALRRCLELKP | |
| | PTS1 COMPLEXED TO | SIGNAL 1 | DNQTALMALAVSFTNESLQRQACEILRDWLRYTPAYAHLVTR | |
| | THE TPR REGION OF | RECEPTOR | ILGSLLSDSLFLEVKELFLAAVRLDPTSIDPDVQCGLGVLFNLS | |
| | HUMAN PEX5 | | GEYDKAVDCFTAALSVRPNDYLLWNKLGATLANGNQSEEAV | |
| | | | AAYRRALELQPGYIRSRYNLGISCINLGAHREAVEHFLEALNM | |
| | | | QRKSGGAMSENIWSTLRLALSMLGQSDAYGAADARDLSTLL | |
| | | | TMFGLPQ | |
| 1g1g | CRYSTAL | PROTEIN | MEMEKEFEQIDKSGSWAAIYQDIRHEASDFPCRVAKLPKNKN | 4 |
| | STRUCTURE OF | TYROSINE | RNRYRDVSPFDHSRIKLHQEDNDYINASLIKMEEAQRSYILTQ | |
| | PROTEIN TYROSINE | PHOSPHATASE | GPLPNTCGHFWEMVWEQKSRGVVMLNRVMEKGSLKCAQY | |
| | PHOSPHATASE 1B | 1B | WPQKEEKEMIFEDTNLKLTLISEDIKSYYTVRQLELENLTTQET | |
| | COMPLEXED WITH A | | REILHFHYTTWPDFGVPESPASFLNFLFKVRESGSLSPEHGPVV | |
| | MONO- | | VHASAGIGRSGTFCLADTCLLLMDKRKDPSSVDIKKVLLEMR | |
| | PHOSPHORYLATED | | KFRMGLIQTADQLRFSYLAVIEGAKFIMGDSSVQDQWKELSH | |
| | PEPTIDE (ETDY(PTR) | | ED | |
| | RKGGKGLL) (SEQ ID | | | |
| | NO: 990)) FROM THE | | | |
| | INSULIN RECEPTOR | | | |
| | KINASE | | | |
| 1hcs | NMR STRUCTURE OF | HUMAN SRC | MDSIQAEEWYFGKITRRESERLLLNAENPRGTFLVRESETTKG | 5 |
| | THE HUMAN SRC SH2 | | AYCLSVSDFDNAKGLNVKHYKIRKLDSGGFYITSRTQFNSLQ | |
| | DOMAIN COMPLEX | | QLVAYYSKHADGLCHRLTTVCP | |
| 1nop | CRYSTAL | tyrosyl-DNA | NPFQFYLTRVSGVKPKYNSGALHIKDILSPLFGTLVSSAQFNY | 6 |
| | STRUCTURE OF | phosphodiesterase | CFDVDWLVKQYPPEFRKKPILLVHGDKREAKAHLHAQAKPY | |
| | HUMAN TYROSYL- | 1 | ENISLCQAKLDIAFGTHHTKMMLLLYEEGLRVVIHTSNLIHAD | |
| | DNA | | WHQKTQGIWLSPLYPRIADGTHKSGESPTHFKANLISYLTAYN | |
| | PHOSPHODIESTERASE | | APSLKEWIDVIHKHDLSETNVYLIGSTPGRFQGSQKDNWGHF | |
| | (TDP1) IN COMPLEX | | RLKKLLKDHASSMSWPVVGQFSSVGSLGADESKWLCSEFKES | |
| | WITH VANADATE, | | MLTLGVPLYLIYPSVENVRTSLEGYPAGGSLPYSIQTAEKQNW | |
| | DNA AND A HUMAN | | LHSYFHKWSAETSGRSNAMPHIKTYMRPSPDFSKIAWFLVTS | |
| | TOPOISOMERASE I- | | ANLSKAAWGALEKNGTQLMIRSYELGVLFLPSALGLDSFKVK | |
| | DERIVED PEPTIDE | | QKFATFPVPYDLPPELYGSKDRPWIWNIPYVKAPDTHGNMW | |
| | | | VPSX | |
| 1nvq | THE COMPLEX | Serine/threonine- | VPFVEDWDLVQTLGEGAYGEVQLAVNRVTEEAVAVKIVDM | 7 |
| | STRUCTURE OF | protein kinase | KNIKKEICINKMLNHENVVKFYGHRREGNIQYLFLEYCSGGEL | |
| | CHECKPOINT KINASE | Chk1 | FDRIEPDIGMPEPDAQRFFHQLMAGVVYLHGIGITHRDIKPENL | |
| | CHK1/UCN-01 | | LLDERDNLKISDFGLATVFRYNNRERLLNKMCGTLPYVAPEL | |
| | | | LKRREFHAEPVDVWSCGIVLTAMLAGELPWDQPSDSCQEYSD | |
| | | | WKEKKTYLNPWKKIDSAPLALLHKILVENPSARITIPDIKKDR | |
| | | | WYNKPLKKGAX | |
| 1pic | PHOSPHATIDYLINOSI | PHOSPHATIDYLI | GSPIPHHDEKTWNVGSSNRNKAENLLRGKRDGTFLVRESSKQ | 8 |
| | TOL 3-KINASE, P85- | NOSITOL 3- | GCYACSVVVDGEVKHCVINKTATGYGFAEPYNLYSSLKELVL | |
| | ALPHA SUBUNIT: C- | KINASE | HYQHTSLVQHNDSLNVTLAYPVYAQQRR | |
| | TERMINAL SH2 | | | |
| | DOMAIN | | | |
| | COMPLEXED WITH A | | | |
| | TYR751 | | | |
| | PHOSPHOPEPTIDE | | | |
| | FROM THE PDGF | | | |
| | RECEPTOR, NMR, | | | |
| | MINIMIZED MEAN | | | |
| | STRUCTURE | | | |
| 1twq | CRYSTAL | peptidoglycan | VCPNIIKRSAWEARETHCPKMNLPAKYVIIIHTAGTSCTVSTDC | 9 |
| | STRUCTURE OF THE | recognition | QTVVRNIQSFHMDTRNFCDIGYHFLVGQDGGVYEGVGWHIQ | |
| | C-TERMINAL PGN- | protein-I-alpha | GSHTYGFNDIALGIAFIGYFVEKPPNAAALEAAQDLIQCAVVE | |
| | BINDING DOMAIN OF | | GYLTPNYLLMGHSDVVNILSPGQALYNIISTWPHFKHX | |
| | HUMAN PGRP- | | | |
| | IALPHA IN COMPLEX | | | |
| | WITH PGN ANALOG | | | |
| | MURAMYL | | | |
| | TRIPEPTIDE | | | |
| 1tzs | CRYSTAL | Cathepsin E | KEPLINYLDMEYFGTISIGSPPQNFTVIFDTGSSNLWVPSVYCT | 10 |
| | STRUCTURE OF AN | | SPACKTHSRFQPSQSSTYSQPGQSFSIQYGTGSLSGIIGADQVS | |
| | ACTIVATION | | VEGLTVVGQQFGESVTEPGQTFVDAEFDGILGLGYPSLAVGG | |
| | INTERMEDIATE OF | | VTPVFDNMMAQNLVDLPMFSVYMSSNPGAGSELIFGGYDHS | |
| | CATHEPSIN E | | HFSGSLNWVPVTKQAYWQIALDNIQVGGTVMFCSEGCQAIV | |
| | | | DTGTSLITGPSDKIKQLQNAIGAAPVDGEYAVECANLNVMPD | |
| | | | VTFTINGVPYTLSPTAYTLLDQFCSSGFQGLDIHPPAGPLWILG | |
| | | | DVFIRQFYSVFDRGNNRVGLAPAV | |
| 1vbs | STRUCTURE OF | CYCLOPHILIN A | VNPTVFFDIAVDGEPLGRVSFELFADKVPKTAENFRALSTGEK | 11 |
| | CYCLOPHILIN | | GFGYKGSCFHRIIPGFMCQGGDFTRHNGTGGKSIYGEKFEDEN | |
| | COMPLEXED WITH | | FILKHTGPGILSMANAGPNINGSQFFICTAKTEWLDGKHVVFG | |
| | (D)ALA CONTAINING | | KVKEGMNIVEAMERFGSRNGKTSKKITIADCGQLE | |
| | TETRAPEPTIDE | | | |
| 1vbt | STRUCTURE OF | CYCLOPHILIN A | VNPTVFFDIAVDGEPLGRVSFELFADKVPKTAENFRALSTGEK | 12 |
| | CYCLOPHILIN | | GFGYKGSCFHRIIPGFMCQGGDFTRHNGTGGKSIYGEKFEDEN | |
| | COMPLEXED WITH | | FILKHTGPGILSMANAGPNINGSQFFICTAKTEWLDGKHVVFG | |
| | SULFUR- | | KVKEGMNIVEAMERFGSRNGKTSKKITIADCGQLE | |
| | SUBSTITUTED | | | |
| | TETRAPEPTIDE AAPF | | | |
| | (SEQ ID NO: 991) | | | |
| 1zys | CO-CRYSTAL | Serine/threonine- | VPFVEDWDLVQTLGEGAYGEVQLAVNRVTEEAVAVKIVDM | 13 |
| | STRUCTURE OF | protein kinase | KRAVDCPENIKKEICINAMLNHENVVKFYGHRREGNIQYLFLE | |
| | CHECKPOINT KINASE | Chk 1 | YCSGGELFDRIEPDIGMPEPDAQRFFHQLMAGVVYLHGIGITH | |
| | CHK1 WITH A | | RDIKPENLLLDERDNLKISDFGLATVFRYNNRERLLNKMCGTL | |
| | PYRROLO-PYRIDINE | | PYVAPELLKRREFHAEPVDVWSCGIVLTAMLAGELPWDQPSD | |
| | INHIBITOR | | SCQEYSDWKEKKTYLNPWKKIDSAPLALLHKILVENPSARITI | |
| | | | PDIKKDRWYNKPLKKGAX | |
| 2g99 | STRUCTURAL BASIS | WD-repeat protein | PVKPNYALKFTLAGHTKAVSSVKFSPNGEWLASSSADKLIKI | 14 |
| | FOR THE SPECIFIC | 5 | WGAYDGKFEKTISGHKLGISDVAWSSDSNLLVSASDDKTLKI | |
| | RECOGNITION OF | | WDVSSGKCLKTLKGHSNYVFCCNFNPQSNLIVSGSFDESVRI | |
| | METHYLATED | | WDVKTGKCLKTLPAHSDPVSAVHFNRDGSLIVSSSYDGLCRI | |
| | HISTONE H3 LYSINE 4 | | WDTASGQCLKTLIDDDNPPVSFVKFSPNGKYILAATLDNTLKL | |
| | BY THE WD-40 | | WDYSKGKCLKTYTGHKNEKYCIFANFSVTGGKWIVSGSEDN | |
| | PROTEIN WDR5 | | LVYIWNLQTKEIVQKLQGHTDVVISTACHPTENIIASAALEND | |
| | | | KTIKLWKSD | |
| 2h13 | CRYSTAL | WD-repeat protein | KPNYALKFTLAGHTKAVSSVKFSPNGEWLASSSADKLIKIWG | 15 |
| | STRUCTURE OF | 5 | AYDGKFEKTISGHKLGISDVAWSSDSNLLVSASDDKTLKIWD | |
| | WDR5/HISTONE H3 | | VSSGKCLKTLKGHSNYVFCCNFNPQSNLIVSGSFDESVRIWDV | |
| | COMPLEX | | KTGKCLKTLPAHSDPVSAVHFNRDGSLIVSSSYDGLCRIWDTA | |
| | | | SGQCLKTLIDDDNPPVSFVKFSPNGKYILAATLDNTLKLWDYS | |
| | | | KGKCLKTYTGHKNEKYCIFANFSVTGGKWIVSGSEDNLVYIW | |
| | | | NLQTKEIVQKLQGHTDVVISTACHPTENIIASAALENDKTIKL | |
| | | | WKSDC | |
| 2m0u | COMPLEX | Na(+)/H(+) | GIDPFTMLPRLCCLEKGPNGYGFHLHGEKGKLGQYIRLVEPGS | 16 |
| | STRUCTURE OF C- | exchange | PAEKAGLLAGDRLVEVNGENVEKETHQQVVSRIRAALNAVR | |
| | TERMINAL CFTR | regulatory cofactor | LLVVDPETDEQLQKLGVQVREELLRAQEAPGQA | |
| | PEPTIDE AND | NHE-RF1 | | |
| | EXTENDED PDZ1 | | | |
| | DOMAIN FROM | | | |
| | NHERF1 | | | |
| 2m0v | COMPLEX | Na(+)/H(+) | GIDPFTMLRPRLCTMKKGPSGYGFNLHSDKSKPGQFIRSVDPD | 17 |
| | STRUCTURE OF C- | exchange | SPAEASGLRAQDRIVEVNGVCMEGKQHGDVVSAIRAGGDET | |
| | TERMINAL CFTR | regulatory cofactor | KLLVVDRETDEFFKKCRVIPSQEHLNGPLPVPFTNGEIQKENSR | |
| | PEPTIDE AND | NHE-RF1 | | |
| | EXTENDED PDZ2 | | | |
| | DOMAIN FROM | | | |
| | NHERF1 | | | |
| 2o9k | WDR5 IN COMPLEX | WD repeat protein | VKPNYALKFTLAGHTKAVSSVKFSPNGEWLASSSADKLIKIW | 18 |
| | WITH | 5 | GAYDGKFEKTISGHKLGISDVAWSSDSNLLVSASDDKTLKIW | |
| | DIMETHYLATED | | DVSSGKCLKTLKGHSNYVFCCNFNPQSNLIVSGSFDESVRIWD | |
| | H3K4 PEPTIDE | | VKTGKCLKTLPAHSDPVSAVHFNRDGSLIVSSSYDGLCRIWDT | |
| | | | ASGQCLKTLIDDNPPVSFVKFSPNGKYILAATLDNTLKLWDYS | |
| | | | KGKCLKTYTGHKNEKYCIFANFSVTGGKWIVSGSEDNLVYIW | |
| | | | NLQTKEIVQKLQGHTDVVISTACHPTENIIASAALENDKTIKL | |
| | | | WKSDC | |
| 2pxj | THE COMPLEX | JmjC domain- | ASESETLNPSARIMTFYPTMEEFRNFSRYIAYIESQGAHRAGLA | 19 |
| | STRUCTURE OF | containing histone | KVVPPKEWKPRASYDDIDDLVIPAPIQQLVTGQSGLFTQYNIQ | |
| | JMJD2A AND | demethylation | KKAMTVREFRKIANSDKYCTPRYSEFEELERKYWKNLTFNPPI | |
| | MONOMETHYLATED | protein 3A | YGADVNGTLYEKHVDEWNIGRLRTILDLVEKESGITIEGVNTP | |
| | H3K36 PEPTIDE | | YLYFGMWKTSFAWHTEDMDLYSINYLHFGEPKSWYSVPPEH | |
| | | | GKRLERLAKGFFPGSAQSCEAFLRHKMTLISPLMLKKYGIPFD | |
| | | | KVTQEAGEFMITFPYGYHAGFNHGFNCAESTNFATRRWIEYG | |
| | | | KQAVLCSCRKDMVKISMDVFVRKFQPERYKLWKAGKDNTVI | |
| | | | DHTLPTPXXX | |
| 2q5a | HUMAN PIN1 BOUND | Peptidyl-prolyl cis- | LPPGWEKAMSRSSGRVYYFNHITNASQWERPSEPARVRCSHL | 20 |
| | TO L-PEPTIDE | trans isomerase | LVKHSQSRRPSSWRQEKITRTKEEALELINGYIQKIKSGEEDFE | |
| | | NIMA-interacting | SLASQFSDCSSAKARGDLGAFSRGQMQKPFEDASFALRTGEM | |
| | | 1 | SGPVFTDSGIHIILRTEX | |
| 3fvh | POLO-LIKE KINASE 1 | Serine/threonine- | DCHLSDMLQQLHSVNASKPSERGLVRQEEAEDPACIPIFWVSK | 21 |
| | POLO BOX DOMAIN | protein kinase | WVDYSDKYGLGYQLCDNSVGVLFNDSTRLILYNDGDSLQYIE | |
| | IN COMPLEX WITH | PLK1 | RDGTESYLTVSSHPNSLMKKITLLKYFRNYMSEHLLKAGANIT | |
| | AC-LHSPTA-NH2 (SEQ | | PREGDELARLPYLRTWFRTRSAIILHLSNGSVQINFFQDHTKLI | |
| | ID NO: 992) PEPTIDE | | LCPLMAAVTYIDEKRDFRTYRLSLLEEYGCCKELASRLRYAR | |
| | | | TMVDKLLSSRSASNRLKAS | |
| 3h11 | ZYMOGEN CASPASE-8: | Caspase-8 | DKVYQMKSKPRGYCLIINNHNFAKAREKVPKLHSIRDRNGTH | 22 |
| | C-FLIPL PROTEASE | | LDAGALTTTFEELHFEIKPHDDCTVEQIYEILKIYQLMDHSNM | |
| | DOMAIN COMPLEX | | DCFICCILSHGDKGIIYGTDGQEAPIYELTSQFTGLKCPSLAGKP | |
| | | | KVFFIQACQGDNYQQTRYIPDEADFLLGMATVNNCVSYRNPA | |
| | | | EGTWYIQSLCQSLRERCPRGDDILTILTEVNYEVSNKGKQMPQ | |
| | | | PTFTLRKKLVFPSD | |
| 3idi | CRYSTAL | 2F5 Fab light chain | ALQLTQSPSSLSASVGDRITITCRASQGVTSALAWYRQKPGSP | 23 |
| | STRUCTURE OF THE | | PQLLIYDASSLESGVPSRFSGSGSGTEFTLTISTLRPEDFATYYC | |
| | HIV-1 CROSS | | QQLHFYPHTFGGGTRVDVRRTVAAPSVFIFPPSDEQLKSGTAS | |
| | NEUTRALIZING | | VVCLLNNFYPREAKVQWKVDNALQSGNSQESVTEQDSKDST | |
| | MONOCLONAL | | YSLSSTLTLSKADYEKHKVYECEVTHQGLSSPVTKSFNRGE | |
| | ANTIBODY 2F5 FAB′ | | | |
| | FRAGMENT IN | | | |
| | COMPLEX WITH GP41 | | | |
| | PEPTIDE ALDKWNQ | | | |
| | (SEQ ID NO: 993) | | | |
| 3n7y | CRYSTAL | Growth factor | MKPHPWFFGKIPRAKAEEMLSKQRHDGAFLIRESESAPGDFSL | 24 |
| | STRUCTURE OF THE | receptor-bound | SVKFGNDVQHFKVLRDGAGKYFLWVVKFNSLNELVDYHRST | |
| | GRB2 SH2 DOMAIN IN | protein 2 | SVSRNQQIFLRDIE | |
| | COMPLEX WITH A 20- | | | |
| | MEMBERED | | | |
| | MACROCYCLIC | | | |
| | LIGAND HAVING THE | | | |
| | SEQUENCE PYVNV | | | |
| | (SEQ ID NO: 994) | | | |
| 3oa6 | HUMAN MSL3 | Male-specific | FKFHSGEKVLCFEPDPTKARVLYDAKIVDVIVGKDEKGRKIPE | 25 |
| | CHROMODOMAIN | lethal 3 homolog | YLIHFNGWNRSWDRWAAEDHVLRDTDENRRLQRKLARKAV | |
| | BOUND TO DNA AND | | A | |
| | H4K20ME1 PEPTIDE | | | |
| 3rq7 | POLO-LIKE KINASE 1 | Serine/threonine- | HLSDMLQQLHSVNASKPSERGLVRQEEAEDPACIPIFWVSKW | 26 |
| | POLO BOX DOMAIN | protein kinase | VDYSDKYGLGYQLCDNSVGVLFNDSTRLILYNDGDSLQYIER | |
| | IN COMPLEX WITH A | PLK1 | DGTESYLTVSSHPNSLMKKITLLKYFRNYMSEHLLKAGAPRLP | |
| | C6H5(CH2)8- | | YLRTWFRTRSAIILHLSNGSVQINFFQDHTKLILCPLMAAVTYI | |
| | DERIVATIZED | | DEKRDFRTYRLSLLEEYGCCKELASRLRYARTMVDKLLSSR | |
| | PEPTIDE INHIBITOR | | | |
| 4dfw | OXIME-BASED POST | Serine/threonine- | HLSDMLQQLHSVNASKPSERGLVRQEEAEDPACIPIFWVSKW | 27 |
| | SOLID-PHASE | protein kinase | VDYSDKYGLGYQLCDNSVGVLFNDSTRLILYNDGDSLQYIER | |
| | PEPTIDE | PLK1 | DGTESYLTVSPNSLMKKITLLKYFRNYMSEHLLKAGANITPRE | |
| | DIVERSIFICATION: | | GDELARLPYLRTWFRTRSAIILHLSNGSVQINFFQDHTKLILCP | |
| | IDENTIFICATION OF | | LMAAVTYIDEKRDFRTYRLSLLEEYGCCKELASRLRYARTMV | |
| | HIGH AFFINITY | | DKLLSSXX | |
| | POLO-LIKE KINASE 1 | | | |
| | (PLK1) POLO-BOX | | | |
| | DOMAIN BINDING | | | |
| | PEPTIDES | | | |
| 4dm9 | THE CRYSTAL | Ubiquitin | MQLKPMEINPEMLNKVLSRLGVAGQWRFVDVLGLEEESLGS | 28 |
| | STRUCTURE OF | carboxyl-terminal | VPAPACALLLLFPLTAQHENFRKKQIEELKGQEVSPKVYFMK | |
| | UBIQUITIN | hydrolase isozyme | QTIGNSCGTIGLIHAVANNQDKLGFEDGSVLKQFLSETEKMSP | |
| | CARBOXY-TERMINAL | L1 | EDRAKCFEKNEAIQAAHDAVAQEGQCRVDDKVNFHFILFNN | |
| | HYDROLASE L1 | | VDGHLYELDGRMPFPVNHGASSEDTLLKDAAKVCREFTEREQ | |
| | (UCHL1) BOUND TO A | | GEVRESAVALCKAA | |
| | TRIPEPTIDE | | | |
| | FLUOROMETHYL | | | |
| | KETONE Z- | | | |
| | VAE(OME)-FMK | | | |
| 4gm9 | CRYSTAL | WD repeat- | TQSKPTPVKPNYALKFTLAGHTKAVSSVKFSPNGEWLASSSA | 29 |
| | STRUCTURE OF | containing protein | DKLIKIWGAYDGKFEKTISGHKLGISDVAWSSDSNLLVSASDD | |
| | HUMAN WD REPEAT | 5 | KTLKIWDVSSGKCLKTLKGHSNYVFCCNFNPQSNLIVSGSFDE | |
| | DOMAIN 5 WITH | | SVRIWDVKTGKCLKTLPAHSDPVSAVHFNRDGSLIVSSSYDGL | |
| | COMPOUND MM-401 | | CRIWDTASGQCLKTLIDNPPVSFVKFSPNGKYILAATLDNTLK | |
| | | | LWDYSKGKCLKTYTGHKNEKYCIFANFSVTGGKWIVSGSED | |
| | | | NLVYIWNLQTKEIVQKLQGHTDVVISTACHPTENIIASAALEN | |
| | | | DKTIKLWKSDC | |
| 4iim | CRYSTAL | Intersectin-1 | LLQAQALYPWRAKKDNHLNFNKNDVITVLEQQDMWWFGEV | 30 |
| | STRUCTURE OF THE | | QGQKGWFPKSYVKLIXXXXX | |
| | SECOND SH3 DOMAIN | | | |
| | OF ITSN1 BOUND | | | |
| | WITH A SYNTHETIC | | | |
| | PEPTIDE | | | |
| 4joh | CFTR ASSOCIATED | Golgi-associated | GPIRKVLLLKEDHEGLGISITGGKEHGVPILISEIHPGQPADRCG | 31 |
| | LIGAND (CAL) PDZ | PDZ and coiled- | GLHVGDAILAVNGVNLRDTKHKEAVTILSQQRGEIEFEVVYV | |
| | DOMAIN BOUND TO | coil motif- | | |
| | PEPTIDE H-ICAL36 | containing protein | | |
| | (ANSRHPTSII (SEQ ID | | | |
| | NO: 995)) | | | |
| 4jr0 | HUMAN | Procaspase-3 | ADNSYKMDYPEMGLCIIINNKNFHKSTGMTSRSGTDVDAANL | 32 |
| | PROCASPASE-3 | | RETFRNLKYEVRNKNDLTREEIVELMRDVSKEDHSKRSSFVC | |
| | BOUND TO AC-DEVD- | | VLLSHGEEGIIFGTNGPVDLKKITNFFRGDRCRSLTGKPKLFIIQ | |
| | CMK (SEQ ID NO: 447) | | ACRGTKIPVEADFLYAYSTAPGYYSWRNSKDGSWFIQSLCAM | |
| | | | LKQYADKLEFMHILTRVNRKVATEFESFSFDATFHAKKQIPCI | |
| | | | VSMLTKELYFYHX | |
| 1a7c | HUMAN | PLASMINOGEN | HHPPSYVAHLASDFGVRVFQQVAQASKDRNVVFSPYGVASV | 33 |
| | PLASMINOGEN | ACTIVATOR | LAMLQLTTGGETQQQIQAAMGFKIDDKGMAPALRHLYKELM | |
| | ACTIVATOR | INHIBITOR TYPE | GPWNKDEISTTDAIFVQRDLKLVQGFMPHFFRLFRSTVKQVDF | |
| | INHIBITOR TYPE-1 IN | 1 | SEVERARFIINDWVKTHTKGMISNLLGKGAVDQLTRLVLVNA | |
| | COMPLEX WITH A | | LYFNGQWKTPFPDSSTHRRLFHKSDGSTVSVPMMAQTNKFN | |
| | PENTAPEPTIDE | | YTEFTTPDGHYYDILELPYHGDTLSMFIAAPYEKEVPLSALTNI | |
| | | | LSAQLISHWKGNMTRLPRLLVLPKFSLETEVDLRKPLENLGM | |
| | | | TDMFRQFQADFTSLSDQEPLHVAQALQKVKIEVNESGTAPEEI | |
| | | | IMDRPFLFVVRHNPTGTVLFMGQVMEPX | |
| 1awq | CYPA COMPLEXED | CYCLOPHILIN A | VNPTVFFDIAVDGEPLGRVSFELFADKVPKTAENFRALSTGEK | 34 |
| | WITH HAGPIA | | GFGYKGSCFHRIIPGFMCQGGDFTRHNGTGGKSIYGEKFEDEN | |
| | (PSEUDO- | | FILKHTGPGILSMANAGPNTNGSQFFICTAKTEWLDGKHVVFG | |
| | SYMMETRIC | | KVKEGMNIVEAMERFGSRNGKTSKKITIADCGQLE | |
| | MONOMER) | | | |
| 1bm2 | GRB2-SH2 DOMAIN IN | PROTEIN | MKPHPWFFGKIPRAKAEEMLSKQRHDGAFLIRESESAPGDFSL | 35 |
| | COMPLEX WITH | (GROWTH | SVKFGNDVQHFKVLRDGAGKYFLWVVKFNSLNELVDYHRST | |
| | CYCLO-[N-ALPHA- | FACTOR | SVSRNQQIFLRDIE | |
| | ACETYL-L-THI | RECEPTOR | | |
| | ALYSYL-O- | BOUND | | |
| | PHOSPHOTYROSYL- | PROTEIN 2) | | |
| | VALYL-ASPARAGYL- | | | |
| | VALYL-PROLYL] | | | |
| | (SEQ ID NO: 997) | | | |
| | (PKF273-791) | | | |
| 1f8h | STRUCTURE OF THE | EPIDERMAL | PWAVKPEDKAKYDAIFDSLSPVNGFLSGDKVKPVLLNSKLPV | 36 |
| | SECOND EPS15 | GROWTH | DILGRVWELSDIDHDGMLDRDEFAVAMFLVYCALEKEPVPM | |
| | HOMOLOGY DOMAIN | FACTOR | SLPPALVPPSKRX | |
| | OF HUMAN EPS15 IN | RECEPTOR | | |
| | COMPLEX WITH | SUBSTRATE 15 | | |
| | PTGSSSTNPFR (SEQ | | | |
| | ID NO: 998) | | | |
| 1ir3 | PHOSPHORYLATED | INSULIN | SSVFVPDEWEVSREKITLLRELGQGSFGMVYEGNARDIIKGEA | 37 |
| | INSULIN RECEPTOR | RECEPTOR | ETRVAVKTVNESASLRERIEFLNEASVMKGFTCHHVVRLLGV | |
| | TYROSINE KINASE IN | | VSKGQPTLVVMELMAHGDLKSYLRSLRPEAENNPGRPPPTLQ | |
| | COMPLEX WITH | | EMIQMAAEIADGMAYLNAKKFVHRDLAARNCMVAHDFTVKI | |
| | PEPTIDE SUBSTRATE | | GDFGMTRDIXETDXXRKGGKGLLPVRWMAPESLKDGVFTTS | |
| | AND ATP ANALOG | | SDMWSFGVVLWEITSLAEQPYQGLSNEQVLKFVMDGGYLDQ | |
| | | | PDNCPERVTDLMRMCWQFNPKMRPTFLEIVNLLKDDLHPSFP | |
| | | | EVSFFHSEENKXXX | |
| 1ywt | CRYSTAL | 14-3-3 protein | MERASLIQKAKLAEQAERYEDMAAFMKGAVEKGEELSCEER | 38 |
| | STRUCTURE OF THE | sigma | NLLSVAYKNVVGGQRAAWRVLSSIEQKSNPEVREYREKVETE | |
| | HUMAN SIGMA | | LQGVCDTVLGLLDSHLIKEAGDAESRVFYLKMKGDYYRYLA | |
| | ISOFORM OF 14-3-3 IN | | EVATGDDKKRIIDSARSAYQEAMDISKKEMPPTNPIRLGLALN | |
| | COMPLEX WITH A | | FSVFHYEIANSPEEAISLAKTTFDEAMADLHTLSEDSYKDSTLI | |
| | MODE-1 | | MQLLRDNLTLWT | |
| | PHOSPHOPEPTIDE | | | |
| 2oqs | STRUCTURE OF THE | Disks large | MEIKLIKGPKGLGFSIAGGVGNQHIPGDNSIYVTKIIEGGAAHK | 39 |
| | HDLG/SAP97 PDZ2 IN | homolog 1 | DGKLQIGDKLLAVNNVCLEEVTHEEAVTALKNTSDFVYLKV | |
| | COMPLEX WITH HPV- | | AKPTGSH | |
| | 18 PAPILLOMA VIRUS | | | |
| | E6 PEPTIDE | | | |
| 2pie | CRYSTAL | E3 ubiquitin- | GAHMAGGRSWCLRRVGMSAGWLLLEDGCEVTVGRGFGVTY | 40 |
| | STRUCTURE OF THE | protein ligase | QLVSKICPLMISRNHCVLKQNPEGQWTIMDNKSLNGVWLNR | |
| | FHA DOMAIN OF | RNF8 | ARLEPLRVYSIHQGDYIQLGVPLENKENAEYEYEVTEEDWETI | |
| | RNF8 IN COMPLEX | | YPCLSPKN | |
| | WITH ITS OPTIMAL | | | |
| | PHOSPHOPEPTIDE | | | |
| 3kqi | CRYSTAL | PHD finger protein | GSMATVPVYCVCRLPYDVTRFMIECDACKDWFHGSCVGVEE | 41 |
| | STRUCTURE OF PHF2 | 2 | EEAPDIDIYHCPNCEKTHGKSTLKKKRTWHXXXXX | |
| | PHD DOMAIN | | | |
| | COMPLEXED WITH | | | |
| | H3K4ME3 PEPTIDE | | | |
| 3tkz | STRUCTURE OF THE | Tyrosine-protein | RRWFHPNITGVEAENLLLTRGVDGSFLARPSKSNPGDFTLSVR | 42 |
| | SHP-2 N-SH2 DOMAIN | phosphatase non- | RNGAVTHIKIQNTGDYYDLYGGEKFATLAELVQYYMEHHGQ | |
| | IN A 1:2 COMPLEX | receptor type 11 | LKEKNGDVIELKYPLN | |
| | WITH RVIPYFVPLNR | | | |
| | PEPTIDE (SEQ ID NO: | | | |
| | 999) | | | |
| 3v4o | HUMAN MALT1 | Mucosa-associated | DQPLAKDKVALLIGNMNYREHPKLKAPLVDVYELTNLLRQL | 43 |
| | (CASPASE DOMAIN) | lymphoid tissue | DFKVVSLLDLTEYEMRNAVDEFLLLLDKGVYGLLYYAGHGY | |
| | IN COMPLEX WITH | lymphoma | ENFGNSFMVPVDAPNPYRSENCLCVQNILKLMQEKETGLNVF | |
| | AN IRREVERSIBLE | translocation | LLDMCRKRNDYDDTIPILDALKVTANIVFGYATCQGAEAFEIQ | |
| | PEPTIDIC INHIBITOR | protein 1 | HSGLANGIFMKFLKDRLLEDKKITVLLDEVAEDMGKCHLTKG | |
| | | | KQALEIRSSLSEKRALTDPIQGTEY | |
| 4jdi | CRYSTAL | Serine/threonine- | SHEQFRAALQLVVDPGDPRSYLDNFIKIGEGSTGIVCIATVRSS | 44 |
| | STRUCTURE OF | protein kinase PAK | GKLVAVKKMDLRKQQRRELLFNEVVIMRDYQHENVVEMYN | |
| | SERINE/THREONINE- | 4 | SYLVGDELWVVMEFLEGGALTDIVTHTRMNEEQIAAVCLAV | |
| | PROTEIN KINASE PAK | | LQALSVLHAQGVIHRDIKSDSILLTHDGRVKLSDFGFCAQVSK | |
| | 4 IN COMPLEX WITH | | EVPRRKXLVGTPYWMAPELISRLPYGPEVDIWSLGIMVIEMV | |
| | PAKTIDE S PEPTIDE | | DGEPPYFNEPPLKAMKMIRDNLPPRLKNLHKVSPSLKGFLDRL | |
| | SUBSTRATE | | LVRDPAQRATAAELLKHPFLAKAGPPASIVPLMRQNRXXX | |
| 4nmp | CFTR ASSOCIATED | Golgi-associated | GPIRKVLLLKEDHEGLGISITGGKEHGVPILISEIHPGQPADRCG | 45 |
| | LIGAND (CAL) PDZ | PDZ and coiled- | GLHVGDAILAVNGVNLRDTKHKEAVTILSQQRGEIEFEVVYV | |
| | DOMAIN BOUND TO | coil motif- | | |
| | PEPTIDE ICAL36(AC- | containing protein | | |
| | K-3) (ANSRWP[AC- | | | |
| | K]SII (SEQ ID NO: | | | |
| | 1000)) | | | |
| 4nnm | TAX-INTERACTING | Tax1-binding | TAVVQRVEIHKLRQGENLILGFSIGGGIDQDPSQNPFSEDKTDK | 46 |
| | PROTEIN-1 (TIP-1) | protein 3 | GIYVTRVSEGGPAEIAGLQIGDKIMQVNGWDMTMVTHDQAR | |
| | PDZ DOMAIN BOUND | | KRLTKRSEEVVRLLVTRQSLQKAVQQ | |
| | TO Y-ICAL36 (YPTSII) | | | |
| | PEPTIDE SEQ ID NO: | | | |
| | 356) | | | |
| 4q6h | CFTR ASSOCIATED | CFTR-associated | GPIRKVLLLKEDHEGLGISITGGKEHGVPILISEIHPGQPADRCG | 47 |
| | LIGAND (CAL) | ligand | GLHVGDAILAVNGVNLRDTKHKEAVTILSQQRGEIEFEVVYV | |
| | BOUND TO LAST 6 | | X | |
| | RESIDUES OF CFTR | | | |
| | (DECAMERIC | | | |
| | PEPTIDE: | | | |
| | ICAL36VQDTRL (SEQ | | | |
| | ID NO: 357)) | | | |
| 1f8a | STRUCTURAL BASIS | PEPTIDYL- | GSHGMADEEKLPPGWEKRMSRSSGRVYYFNHITNASQWERP | 48 |
| | FOR THE | PROLYL CIS- | SEPARVRCSHLLVKHSQSRRPSSWRQEKITRTKEEALELINGYI | |
| | PHOSPHOSERINE- | TRANS | QKIKSGEEDFESLASQFSDCSSAKARGDLGAFSRGQMQKPFED | |
| | PROLINE | ISOMERASE | ASFALRTGEMSGPVFTDSGIHIILRTE | |
| | RECOGNITION BY | NIMA- | | |
| | GROUP IV WW | INTERACTING 1 | | |
| | DOMAINS | | | |
| 1l7z | CRYSTAL | CALMODULIN | QLTEEQIAEFKEAFSLFDKDGDGTITTKELGTVMRSLGQNPTE | 49 |
| | STRUCTURE OF | | AELQDMINEVDADGNGTIDFPEFLTMMARKMKDSEEEIREAF | |
| | CA2+/CALMODULIN | | RVFDKDGNGYISAAELRHVMTNLGEKLTDEEVDEMIREADID | |
| | COMPLEXED WITH | | GDGQVNYEEFVQMMTXXXX | |
| | MYRISTOYLATED | | | |
| | CAP-23/NAP-22 | | | |
| | PEPTIDE | | | |
| 2gfa | DOUBLE TUDOR | Jumonji domain- | LQSITAGQKVISKHKNGRFYQCEVVRLTTETFYEVNFDDGSFS | 50 |
| | DOMAIN COMPLEX | containing protein | DNLYPEDIVSGPPAEGEVVQVRWTDGQVYGAKFVASHPIQM | |
| | STRUCTURE | 2A | YQVEFEDGSQLVVKRDDVYTLDEELP | |
| 2ovr | STRUCTURE OF THE | F-box/WD repeat | TQVKHMMQVIEPQFQRDFISLLPKELALYVLSFLEPKDLLQAA | 51 |
| | SKP1-FBW7- | protein 7 | QTCRYWRILAEDNLLWREKCKEEGIDEPLHIKRVIKPGFIHSP | |
| | CYCLINEDEGN | | WKSAYIRQHRIDTNWRRGELKSPKVLKGHDDHVITCLQFCGN | |
| | COMPLEX | | RIVSGSDDNTLKVWSAVTGKCLRTLVGHTGGVWSSQMRDNII | |
| | | | ISGSTDRTLKVWNAETGECIHTLYGHTSTVRCMHLHEKRVVS | |
| | | | GSRDATLRVWDIETGQCLHVLMGHVAAVRCVQYDGRRVVS | |
| | | | GAYDFMVKVWDPETETCLHTLQGHTNRVYSLQFDGIHVVSG | |
| | | | SLDTSIRVWDVETGNCIHTLTGHQSLTSGMELKDNILVSGNAD | |
| | | | STVKIWDIKTGQCLQTLQGPNKHQSAVTCLQFNKNFVITSSDD | |
| | | | GTVKLWDLKTGEFIRNLVTLESGGSGGVVWRIRASNTKLVCA | |
| | | | VGSRNGTEETKLLVLDFDVDM | |
| 3jpv | CRYSTAL | Proto-oncogene | PLESQYQVGPLLGSGGFGSVYSGIRVSDNLPVAIKHVEKDRIS | 52 |
| | STRUCTURE OF | serine/threonine- | DWGELPNGTRVPMEVVLLKKVSSGFSGVIRLLDWFERPDSFV | |
| | HUMAN PROTO- | protein kinase Pim- | LILERPEPVQDLFDFITERGALQEELARSFFWQVLEAVRHCHN | |
| | ONCOGENE SERINE | 1 | CGVLHRDIKDENILIDLNRGELKLIDFGSGALLKDTVYTDFDG | |
| | THREONINE KINASE | | TRVYSPPEWIRYHRYHGRSAAVWSLGILLYDMVCGDIPFEHD | |
| | (PIM1) IN COMPLEX | | EEIIGGQVFFRQRVSSECQHLIRWCLALRPSDRPTFEEIQNHPW | |
| | WITH A CONSENSUS | | MQDVLLPQETAEIHLHX | |
| | PEPTIDE AND A | | | |
| | PYRROLO[2,3- | | | |
| | A]CARBAZOLE | | | |
| | LIGAND | | | |
| 4E35 | CRYSTAL | Golgi-associated | GPIRKVLLLKEDHEGLGISITGGKEHGVPILISEIHPGQPADRCG | 53 |
| | STRUCTURE OF CFTR | PDZ and coiled- | GLHVGDAILAVNGVNLRDTKHKEAVTILSQQRGEIEFEVVYV | |
| | ASSOCIATED LIGAND | coil motif- | | |
| | (CAL) PDZ DOMAIN | containing protein | | |
| | BOUND TO ICAL36-L | | | |
| | (ANSRWPTSIL) | | | |
| | PEPTIDE (SEQ ID NO: | | | |
| | 1001) | | | |
| 4po2 | CRYSTAL | Heat shock 70 kDa | ENVQDLLLLDVAPLSLGLETAGGVMTALIKRNSTIPTKQTQIF | 54 |
| | STRUCTURE OF THE | protein 1A/1B | TTYSDNQPGVLIQVYEGERAMTKDNNLLGRFELSGIPPAPRGV | |
| | STRESS-INDUCIBLE | | PQIEVTFDIDANGILNVTATDKSTGKANKITITNDKGRLSKEEI | |
| | HUMAN HEAT SHOCK | | ERMVQEAEKYKAEDEVQRERVSAKNALESYAFNMKSAVEDE | |
| | PROTEIN HSP70 | | GLKGKISEADKKKVLDKCQEVISWLDANTLAEKDEFEHKRKE | |
| | SUBSTRATE-BINDING | | LEQVCNPIISGLYQG | |
| | DOMAIN IN | | | |
| | COMPLEX WITH | | | |
| | PEPTIDE SUBSTRATE | | | |
| 1cwd | HUMAN P56LCK | P56LCK | GSWFFKNLSRKDAERQLLAPGNTHGSFLIRESESTAGSFSLSV | 55 |
| | TYROSINE KINASE | TYROSINE | RDFDQNQGEVVKHYKIRNLDNGGFYISPRITFPGLHELVRHYT | |
| | COMPLEXED WITH | KINASE | NASDGLCTRLSR | |
| | PHOSPHOPEPTIDE | | | |
| 2qbw | THE CRYSTAL | PDZ-Fibronectin | SPELGFSISGGVGGRGNPFRPDDDGIFVTRVQPEGPASKLLQPG | 56 |
| | STRUCTURE OF PDZ- | fusion protein | DKIIQANGYSFINIEHGQAVSLLKTFQNTVELIIVREVGNGAKQ | |
| | FIBRONECTIN | | EIRVRVEKDSSVPTNLEVVAATPTSLLISWDASYYGVSYYRIT | |
| | FUSION PROTEIN | | YGETGGNSPVQEFTVPYSSSTATISGLKPGVDYTITVYAYSDY | |
| | | | YGSHHYSPISINYRT | |
| 3ch8 | THE CRYSTAL | fusion protein | SPELGFSISGGVGGRGNPFRPDDDGIFVTRVQPEGPASKLLQPG | 57 |
| | STRUCTURE OF PDZ- | PDZ-Fibronectin | DKIIQANGYSFINIEHGQAVSLLKTFQNTVELIIVREGAKQEIRV | |
| | FIBRONECTIN | | RVEKDGGSGGVSSVPTNLEVVAATPTSLLISWDAYRELPVSY | |
| | FUSION PROTEIN | | YRITYGETGGNSPVQEFTVPGSKSTATISGLKPGVDYTITVYA | |
| | | | HYNYHYYSSPISINYRX | |
| 3hna | CRYSTAL | Histone-lysine N- | GSNSQVWSALQMSKALPSPVERIVSRDIARGYERIPIPCVNAV | 58 |
| | STRUCTURE OF | methyltransferase, | DSEPCPSNYKYVSQNCVTSPMNIDRNITHLQYCVCIDDCSSSN | |
| | CATALYTIC DOMAIN | H3 lysine-9 | CMCGQLSMRCWYDKDGRLLPEFNMAEPPLIFECNHACSCWR | |
| | OF HUMAN | specific 5 | NCRNRVVQNGLRARLQLYRTRDMGWGVRSLQDIPPGTFVCE | |
| | EUCHROMATIC | | YVGELISDSEADVREEDSYLFDLDNKDGEVYCIDARFYGNVS | |
| | HISTONE | | RFINHHCEPNLVPVRVFMAHQDLRFPRIAFFSTRLIEAGEQLGF | |
| | METHYLTRANSFERASE | | DYGERFWDIKGKLFSCRCGSPKCRHSXXXXX | |
| | 1 IN COMPLEX | | | |
| | WITH SAH AND | | | |
| | MONO-METHYLATED | | | |
| | H3K9 PEPTIDE | | | |
| 4e3b | CRYSTAL | Tax1-binding | AVVQRVEIHKLRQGENLILGFSIGGGIDQDPSQNPFSEDKTDK | 59 |
| | STRUCTURE OF TAX- | protein 3 | GIYVTRVSEGGPAEIAGLQIGDKIMQVNGWDMTMVTHDQAR | |
| | INTERACTING | | KRLTKRSEEVVRLLVTRQ | |
| | PROTEIN-1 (TIP-1) | | | |
| | PDZ DOMAIN BOUND | | | |
| | TO ICAL36-L | | | |
| | (ANSRWPTSIL) | | | |
| | PEPTIDE | | | |
| 4okw | CRYSTAL | Androgen receptor | PIFLNVLEAIEPGVVCAGHDNNQPDSFAALLSSLNELGERQLV | 60 |
| | STRUCTURE OF | | HVVKWAKALPGFRNLHVDDQMAVIQYSLMGLMVFAMGWR | |
| | W741L-AR-LBD | | SFTNVNSAMLYFAPDLVFNEYRMHKSRMYSQCVRMRHLSQE | |
| | BOUND WITH CO- | | FGWLQITPQEFLCMKALLLFSIIPVDGLKNQKFFDELRMNYIK | |
| | REGULATOR PEPTIDE | | ELDRIIACSRRFYQLTKLLDSVQPIARELHQFTFDLLIKSHMVS | |
| | | | VDFPEMMAEIISVQVPKILSGKVKPIYFHX | |
| 1a9e | DECAMER-LIKE | HLA CLASS I | GSHSMRYFYTAMSRPGRGEPRFIAVGYVDDTQFVRFDSDAAS | 61 |
| | CONFORMATION OF | HISTOCOMPATI | PRTEPRAPWIEQEGPEYWDRNTQIFKTNTQTYRESLRNLRGYY | |
| | A NANO-PEPTIDE | BILITY | NQSEAGSHIIQRMYGCDLGPDGRLLRGHDQSAYDGKDYIALN | |
| | BOUND TO HLA- | ANTIGEN, B-35 | EDLSSWTAADTAAQITQRKWEAARVAEQLRAYLEGLCVEWL | |
| | B3501 DUE TO | B*3501 (ALPHA | RRYLENGKETLQRADPPKTHVTHHPVSDHEATLRCWALGFYP | |
| | NONSTANDARD | CHAIN) | AEITLTWQRDGEDQTQDTELVETRPAGDRTFQKWAAVVVPS | |
| | POSITIONING OF THE | | GEEQRYTCHVQHEGLPKPLTLRWEPS | |
| | C-TERMINUS | | | |
| 1cf0 | HUMAN PLATELET | PROTEIN | GWNAYIDNLMADGTCQDAAIVGYKDSPSVWAAVPGKTFVNI | 62 |
| | PROFILIN | (PROFILIN) | TPAEVGVLVGKDRSSFYVNGLTLGGQKCSVIRDSLLQDGEFS | |
| | COMPLEXED WITH | | MDLRTKSTGGAPTFNVTVTKTDKTLVLLMGKEGVHGGLINK | |
| | AN L-PRO10- | | KCYEMASHLRRSQY | |
| | IODOTYROSINE | | | |
| | PEPTIDE (SEQ ID NO: | | | |
| | 1002) | | | |
| 1eeo | CRYSTAL | PROTEIN | EMEKEFEQIDKSGSWAAIYQDIRHEASDFPCRVAKLPKNKNR | 63 |
| | STRUCTURE OF | TYROSINE | NRYRDVSPFDHSRIKLHQEDNDYINASLIKMEEAQRSYILTQG | |
| | PROTEIN TYROSINE | PHOSPHATASE | PLPNTCGHFWEMVWEQKSRGVVMLNRVMEKGSLKCAQYWP | |
| | PHOSPHATASE 1B | 1B | QKEEKEMIFEDTNLKLTLISEDIKSYYTVRQLELENLTTQETRE | |
| | COMPLEXED WITH | | ILHFHYTTWPDFGVPESPASFLNFLFKVRESGSLSPEHGPVVVH | |
| | ACETYL-E-L-E-F- | | SSAGIGRSGTFCLADTCLLLMDKRKDPSSVDIKKVLLEMRKFR | |
| | PTYR-M-D-Y-E-NH2 | | MGLIQTADQLRFSYLAVIEGAKFIMGDSSVQDQWKELSHEDX | |
| | (SEQ ID NO: 1003) | | | |
| 1hsa | THE THREE- | CLASS I | GSHSMRYFHTSVSRPGRGEPRFITVGYVDDTLFVRFDSDAASP | 64 |
| | DIMENSIONAL | HISTOCOMPATI | REEPRAPWIEQEGPEYWDRETQICKAKAQTDREDLRTLLRYY | |
| | STRUCTURE OF HLA- | BILITY | NQSEAGSHTLQNMYGCDVGPDGRLLRGYHQDAYDGKDYIAL | |
| | B27 AT 2.1 | ANTIGEN (HLA- | NEDLSSWTAADTAAQITQRKWEAARVAEQLRAYLEGECVEW | |
| | ANGSTROMS | B*2705) | LRRYLENGKETLQRADPPKTHVTHHPISDHEATLRCWALGFY | |
| | RESOLUTION | | PAEITLTWQRDGEDQTQDTELVETRPAGDRTFQKWAAVVVPS | |
| | SUGGESTS A | | GEEQRYTCHVQHEGLPKPLTLRWEP | |
| | GENERAL | | | |
| | MECHANISM FOR | | | |
| | TIGHT PEPTIDE | | | |
| | BINDING TO MHC | | | |
| 3fe7 | CRYSTAL | Mdm4 protein | QVRPKLPLLKILHAAGAQGEMFTVKEVMHYLGQYIMVKQLY | 65 |
| | STRUCTURE OF | | DQQEQHMVYCGGDLLGELLGRQSFSVKDPSPLYDMLRKNLV | |
| | HDMX BOUND TO | | T | |
| | THE P53- | | | |
| | PEPTIDOMIMETIC | | | |
| | AC-PHE-MET- AIB- | | | |
| | PMP-TRP-GLU-AC3C- | | | |
| | LEU-NH2 AT 1.35A | | | |
| 3kpq | CRYSTAL | HLA class I | GSHSMRYFYTAMSRPGRGEPRFITVGYVDDTLFVRFDSDATSP | 66 |
| | STRUCTURE OF HLA | histocompatibility | RKEPRAPWIEQEGPEYWDRETQISKTNTQTYRENLRTALRYY | |
| | B*4405 IN COMPLEX | antigen, B-44 alpha | NQSEAGSHIIQRMYGCDVGPDGRLLRGYDQYAYDGKDYIAL | |
| | WITH EEYLKAWTF | chain | NEDLSSWTAADTAAQITQRKWEAARVAEQDRAYLEGLCVES | |
| | (SEQ ID NO: 376), A | | LRRYLENGKETLQRADPPKTHVTHHPISDHEVTLRCWALGFY | |
| | MIMOTOPE | | PAEITLTWQRDGEDQTQDTELVETRPAGDRTFQKWAAVVVPS | |
| | | | GEEQRYTCHVQHEGLPKPLTLRWEP | |
| 4jij | CRYSTAL | Matrix | GFQTFEGDLKWHHHNITYWIQNYSEDLPRAVIDDAFARAFAL | 67 |
| | STRUCTURE OF AN | metalloproteinase- | WSAVTPLTFTRVYSRDADIVIQFGVAEHGDGYPFDGKDGLLA | |
| | INACTIVE MUTANT | 9 | HAFPPGPGIQGDAHFDDDELWSLGKGVGYSLFLVAAHAFGH | |
| | OF MMP-9 | | ALGLDHSSVPEALMYPMYRFTEGPPLHKDDVNGIRHLYGXX | |
| | CATALYTIC DOMAIN | | XXXXXX | |
| | IN COMPLEX WITH A | | | |
| | FLUOROGENIC | | | |
| | SYNTHETIC PEPTIDIC | | | |
| | SUBSTRATE | | | |
| 1aqc | X11 PTB DOMAIN- | X11 | EDLIDGIIFAANYLGSTQLLSDKTPSKNVRQAQEAVSRIKAQK | 68 |
| | 10MER PEPTIDE | | LTEVDLFILTQRIKVLNADTQETDHPLRTISYIADIGNIVVLARR | |
| | COMPLEX | | RYKICHVFESEDAQLIAQSIGQAFSVAYQEFLR | |
| 2axi | HDM2 IN COMPLEX | Ubiquitin-protein | EQETLVRPKPLLLKLLKSVGAQKDTYTMKEVLFYLGQYIMTK | 69 |
| | WITH A BETA- | ligase E3 Mdm2 | RLYDEKQQHIVYCSNDLLGDLFGVPSFSVKEHRKIYTMIYRNL | |
| | HAIRPIN | | VVVNQQEX | |
| 2o02 | PHOSPHORYLATION | 14-3-3 protein | MDKNELVQKAKLAEQAERYDDMAACMKSVTEQGAELSNEE | 70 |
| | INDEPENDENT | zeta/delta | RNLLSVAYKNVVGARRSSWRVVSSIEQKTEGAEKKQQMARE | |
| | INTERACTIONS | | YREKIETELRDICNDVLSLLEKFLIPNASQAESKVFYLKMKGD | |
| | BETWEEN 14-3-3 AND | | YYRYLAEVAAGDDKKGIVDQSQQAYQEAFEISKKEMQPTHPI | |
| | EXOENZYME S: FROM | | RLGLALNFSVFYYEILNSPEKACSLAKTAFDEAIAELDTLSEES | |
| | STRUCTURE TO | | YKDSTLIMQLLRDNLTLWTSX | |
| | PATHOGENESIS | | | |
| 3p9g | CRYSTAL | Tumor | AVSESQLKKMVSKYKYRDLTVRETVNVITLYKDLKPVLDSYG | 71 |
| | STRUCTURE OF THE | susceptibility gene | GSRELMNLTGTIPVPYRGNTYNIPICLWLLDTYPYNPPICFVKP | |
| | TSG101 UEV DOMAIN | 101 protein | TSSMTIKTGKHVDANGKIYLPYLHEWKHPQSDLLGLIQVMIV | |
| | IN COMPLEX WITH | | VFGDEPPVFSRP | |
| | FA459 PEPTIDE | | | |
| 3qhr | STRUCTURE OF A | Cell division | GHMENFQKVEKIGEGTYGVVYKARNKLTGEVVALKKIRLDT | 72 |
| | PCDK2/CYCLINA | protein kinase 2 | ETEGVPSTAIREISLLKELNHPNIVKLLDVIHTENKLYLVFEFLH | |
| | TRANSITION-STATE | | QDLKKFMDASALTGIPLPLIKSYLFQLLQGLAFCHSHRVLHRD | |
| | MIMIC | | LKPQNLLINTEGAIKLADFGLARAFGVPVRTYHEVVTLWYRA | |
| | | | PEILLGCKYYSTAVDIWSLGCIFAEMVTRRALFPGDSEIDQLFR | |
| | | | IFRTLGTPDEVVWPGVTSMPDYKPSFPKWARQDFSKVVPPLD | |
| | | | EDGRSLLSQMLHYDPNKRISAKAALAHPFFQDVTKPVPHLXX | |
| | | | XXX | |
| 3sfj | CRYSTAL | Tax1-binding | TAVVQRVEIHKLRQGENLILGFSIGGGIDQDPSQNPFSEDKTDK | 73 |
| | STRUCTURE OF TAX- | protein 3 | GIYVTRVSEGGPAEIAGLQIGDKIMQVNGWDMTMVTHDQAR | |
| | INTERACTING | | KRLTKRSEEVVRLLVTRQ | |
| | PROTEIN-1 (TIP-1) | | | |
| | PDZ DOMAIN BOUND | | | |
| | TO ICAL36 INHIBITOR | | | |
| | PEPTIDE | | | |
| 4j9c | CRYSTAL | Tyrosine-protein | PNLFVALYDFVASGDNTLSITKGEKLRVLGYNQTGEWCEAQT | 74 |
| | STRUCTURE OF THE | kinase ABL1 | KNGQGWVPSNYITPVNXX | |
| | ABL-SH3 DOMAIN | | | |
| | H59Q-N96T MUTANT | | | |
| | COMPLEXED WITH | | | |
| | THE DESIGNED HIGH- | | | |
| | AFFINITY PEPTIDE | | | |
| | LIGAND P17 | | | |
| 4rxz | CRYSTAL | Protein Mdm4 | NQVRPKLPLLKILHAAGAQGEMFTVKEVMHYLGQYIMVKQL | 75 |
| | STRUCTURE OF | | YDQQEQHMVYCGGDLLGELLGRQSFSVKDPSPLXDMLRKNL | |
| | MDMX | | VT | |
| | PHOSPORYLATED | | | |
| | TYR99 IN COMPLEX | | | |
| | WITH A 12-MER | | | |
| | PEPTIDE | | | |
| 1ov3 | STRUCTURE OF THE | Neutrophil cytosol | SPEFIILQTYRAIADYEKTSGSEMALSTGDVVEVVEKSESGWW | 76 |
| | P22PHOX-P47PHOX | factor 1 | FCQMKAKRGWIPASFLEPLDSPDEPNYAGEPYVAIKAYTAVE | |
| | COMPLEX | | GDEVSLLEGEAVEVIHKLLDGWWVIRKDDVTGYFPSMYLQK | |
| | | | S | |
| 2p81 | CRYSTAL | nmAb 2F5, light | ALQLTQSPSSLSASVGDRITITCRASQGVTSALAWYRQKPGSP | 77 |
| | STRUCTURE OF THE | chain | PQLLIYDASSLESGVPSRFSGSGSGTEFTLTISTLRPEDFATYYC | |
| | HIV-1 CROSS | | QQLHFYPHTFGGGTRVDVRRTVAAPSVFIFPPSDEQLKSGTAS | |
| | NEUTRALIZING | | VVCLLNNFYPREAKVQWKVDNALQSGNSQESVTEQDSKDST | |
| | MONOCLONAL | | YSLSSTLTLSKADYEKHKVYECEVTHQGLSSPVTKSFNRGEC | |
| | ANTIBODY 2F5 IN | | | |
| | COMPLEX WITH GP41 | | | |
| | PEPTIDE | | | |
| | ELLELDKWASLWN | | | |
| | (SEQ ID NO: 1005) | | | |
| 3p72 | STRUCTURE OF | Platelet | HPICEVSKVASHLEVNCDKRQLTALPPDLPKDTTILHLSENLL | 78 |
| | PLATELET | glycoprotein Ib | YTFSLATLMPYTRLTQLNLDRCELTKLQVDGTLPVLGTLDLS | |
| | GLYCOPROTEIN 1B | alpha chain | HNQLQSLPLLGQTLPALTVLDVSFNRLTSLPLGALRGLGELQE | |
| | ALPHA WITH A | | LYLKGNELKTLPPGLLTPTPKLEKLSLANNQLTELPAGLLNGL | |
| | BOUND PEPTIDE | | ENLDTLLLQENSLYTIPKGFFGSHLLPFAFLHGNPWLCNCEILY | |
| | INHIBITOR | | FRRWLQDNAENVYVWKQGVDVKAMTSNVASVQCDNSDKFP | |
| | | | VYKYPGKGCPLVPRX | |
| 1y98 | STRUCTURE OF THE | Breast cancer type | RMSMVVSGLTPEEFMLVYKFARKHHITLTNLITEETTHVVMK | 79 |
| | BRCT REPEATS OF | 1 susceptibility | TDAEFVCERTLKYFLGIAGGKWVVSYFWVTQSIKERKMLNEH | |
| | BRCA1 BOUND TO A | protein | DFEVRGDVVNGRNHQGPKRARESQDRKIFRGLEICCYGPFTN | |
| | CTIP | | MPTDQLEWMVQLCGASVVKELSSFTLGTGVHPIVVVQPDAW | |
| | PHOSPHOPEPTIDE. | | TEDNGFHAIGQMCEAPVVTREWVLDSVALYQCQELDTYLIPQ | |
| | | | IPX | |
| 21gk | NMR STRUCTURE OF | E3 ubiquitin- | SGPSCKHCKDDVNRLCRVCACHLCGGRQDPDKQLMCDECD | 80 |
| | UHRF1 PHD DOMAINS | protein ligase | MAFHIYCLDPPLSSVPSEDEWYCPECRNDXXX | |
| | IN A COMPLEX WITH | UHRF1 | | |
| | HISTONE H3 PEPTIDE | | | |
| 3eqy | CRYSTAL | Mdm4 protein | NQVRPKLPLLKILHAAGAQGEMFTVKEVMHYLGQYIMVKQL | 81 |
| | STRUCTURE OF | | YDAAAQHMVYCGGDLLGELLGRQSFSVKDPSPLYDMLRKNL | |
| | HUMAN MDMX IN | | VTX | |
| | COMPLEX WITH A 12- | | | |
| | MER PEPTIDE | | | |
| | INHIBITOR | | | |
| 2oj2 | NMR STRUCTURE | Hematopoetic Cell | GPLGSPGPNSHNSNTPGIREAGSEDIIVVALYDYEAIHHEDLSF | 82 |
| | ANALYSIS OF THE | Kinase, SH3 | QKGDQMVVLEESGEWWKARSLATRKEGYIPSNYVARVDSLE | |
| | HEMATOPOETIC | domain | T | |
| | CELL KINASE SH3 | | | |
| | DOMAIN | | | |
| | COMPLEXED WITH | | | |
| | AN ARTIFICIAL HIGH | | | |
| | AFFINITY LIGAND | | | |
| | (PD1) | | | |
| 3d8c | FACTOR INHIBITING | Hypoxia-inducible | VASGSGEPREEAGALGPAWDESQLRSYSFPTRPIPRLSQSDPR | 83 |
| | HIF-1 ALPHA D201G | factor 1 alpha | AEELIENEEPVVLTDTNLVYPALKWDLEYLQENIGNGDFSVYS | |
| | MUTANT IN | inhibitor | ASTHKFLYYDEKKMANFQNFKPRSNREEMKFHEFVEKLQDIQ | |
| | COMPLEX WITH | | QRGGEERLYLQQTLNDTVGRKIVMDFLGFNWNWINKQQGKR | |
| | ZN(II), ALPHA- | | GWGQLTSNLLLIGMEGNVTPAHYGEQQNFFAQIKGYKRCILF | |
| | KETOGLUTARATE | | PPDQFECLYPYPVHHPCDRQSQVDFDNPDYERFPNFQNVVGY | |
| | AND HIF-1 ALPHA | | ETVVGPGDVLYIPMYWWHHIESLLNGGITITVNFWYKGAPTP | |
| | 19 MER | | KRIEYPLKAHQKVAIMRNIEKMLGEALGNPQEVGPLLNTMIK | |
| | | | GRYNXX | |
| 2qki | HUMAN C3C IN | Complement C3 | PMYSIITPNILRLESEETMVLEAHDAQGDVPVTVTVHDFPGKK | 84 |
| | COMPLEX WITH THE | | LVLSSEKTVLTPATNHMGNVTFTIPANRGRNKFVTVQATFGT | |
| | INHIBITOR | | QVVEKVVLVSLQSGYLFIQTDKTIYTPGSTVLYRIFTVNHKLLP | |
| | COMPSTATIN | | VGRTVMVNIENPEGIPVKQDSLSSQNQLGVLPLSWDIPELVNM | |
| | | | GQWKIRAYYENSPQQVFSTEFEVKEYVLPSFEVIVEPTEKFYYI | |
| | | | YNEKGLEVTITARFLYGKKVEGTAFVIFGIQDGEQRISLPESLK | |
| | | | RIPIEDGSGEVVLSRKVLLDGVQRAEDLVGKSLYVSATVILHS | |
| | | | GSDMVQAERSGIPIVTSPYQIHFTKTPKYFKPGMPFDLMVFVT | |
| | | | NPDGSPAYRVPVAVQGEDTVQSLTQGDGVAKLSINTHPSQKP | |
| | | | LSITVRTKKQELSEAEQATRTMQALPYSTVGNSNNYLHLSVL | |
| | | | RTELRPGETLNVNFLLRMDRAHEAKIRYYTYLIMNKGRLLKA | |
| | | | GRQVREPGQDLVVLPLSITTDFIPSFRLVAYYTLIGASGQREVV | |
| | | | ADSVWVDVKDSCVGSLVVKSGQSQPVPGQQMTLKIEGDHGA | |
| | | | RVVLVAVDKGVFVLNKKNKLTQSKIWDVVEKADIGCTPGSG | |
| | | | KDYAGVFSDAGLTFTSSSGQQTAQRAELQCPQPXXXXXXXX | |
| | | | X | |
| 4fmq | CRYSTAL | Mitogen-activated | AGPEMVRGQVFDVGPRYTNLSYIGEGAYGMVCSAYDNVNK | 85 |
| | STRUCTURE OF | protein kinase 1 | VRVAIKKISPFEHQTYCQRTLREIKILLRFRHENIIGINDIIRAPTI | |
| | HUMAN ERK2 | | EQMKDVYIVQDLMETDLYKLLKTQHLSNDHICYFLYQILRGL | |
| | COMPLEXED WITH A | | KYIHSANVLHRDLKPSNLLLNTTCDLKICDFGLARVADPDHD | |
| | MAPK DOCKING | | HTGFLTEYVATRWYRAPEIMLNSKGYTKSIDIWSVGCILAEML | |
| | PEPTIDE | | SNRPIFPGKHYLDQLNHILGILGSPSQEDLNCIINLKARNYLLSL | |
| | | | PHKNKVPWNRLFPNADSKALDLLDKMLTFNPHKRIEVEQALA | |
| | | | HPYLEQYYDPSDEPIAEAPFKFDMELDDLPKEKLKELIFEETAR | |
| | | | FQPGYX | |
| 2m3m | SOLUTION | Disks large | MEIKLIKGPKGLGFSIAGGVGNQHIPGDNSIYVTKIIEGGAAHK | 86 |
| | STRUCTURE OF A | homolog 1 | DGKLQIGDKLLAVNNVCLEEVTHEEAVTALKNTSDFVYLKV | |
| | COMPLEX | | AKPTGSHHH | |
| | CONSISTING OF | | | |
| | HDLG/SAP-97 | | | |
| | RESIDUES 318-406 | | | |
| | AND HPV51 | | | |
| | ONCOPROTEIN E6 | | | |
| | RESIDUES 141-151 | | | |
| 5ic3 | CAL PDZ DOMAIN | Golgi-associated | GPIRKVLLLKEDHEGLGISITGGKEHGVPILISEIHPGQPADRCG | 87 |
| | WITH PEPTIDE AND | PDZ and coiled- | GLHVGDAILAVNGVNLRDTKHKEAVTILSQQRGEIEFEVVYV | |
| | INHIBITOR | coil motif- | X | |
| | | containing protein | | |
| 2kpl | MAGI-1 PDZ1/E6CT | Membrane- | GAMGKPFFTRNPSELKGKFIHTKLRKSSRGFGFTVVGGDEPDE | 88 |
| | | associated | FLQIKSLVLDGPAALDGKMETGDVIVSVNDTCVLGHTHAQVV | |
| | | guanylate kinase, | KIFQSIPIGASVDLELCRGYPLPFDPDDPNTSLVTSVAILDKEP | |
| | | WW and PDZ | | |
| | | domain-containing | | |
| | | protein 1 | | |
| 4pz5 | CRYSTAL | Fibronectin | EETCFDKYTGNTYRVGDTYERPKDSMIWDCTCIGAGRGRISC | 89 |
| | STRUCTURE OF THE | | TIANRCHEGGQSYKIGDTWRRPHETGGYMLECVCLGNGKGE | |
| | SECOND AND THIRD | | WTCKPI | |
| | FIBRONECTIN F1 | | | |
| | MODULES IN | | | |
| | COMPLEX WITH A | | | |
| | FRAGMENT OF BBK32 | | | |
| | FROM BORRELIA | | | |
| | BURGDORFERI | | | |
| 217u | STRUCTURE OF CEL- | Advanced | GSAQNITARIGEPLVLKCKGAPKKPPQRLEWKLNTGRTEAWK | 90 |
| | PEP-RAGE V DOMAIN | glycosylation end | VLSPQGGGPWDSVARVLPNGSLFLPAVGIQDEGIFRCQAMNR | |
| | COMPLEX | product-specific | NGKETKSNYRVRVYQIPGKPE | |
| | | receptor | | |
| 6hy7 | CRYSTAL | Neuronal | DGKYAQKLFNDLFEDYSNALRPVEDTDKVLNVTLQITLSQIK | 91 |
| | STRUCTURE OF | acetylcholine | DMDERNQILTAYLWIRQIWHDAYLTWDRDQYDGLDSIRIPSD | |
| | ALPHA9 NACHR | receptor subunit | LVWRPDIVLYNKADDEEPVNTNVVLRYDGLITWDAPAITKSS | |
| | EXTRACELLULAR | alpha-9 | CVVDVTYFPFDNQQCNLTFGSWTYNGNQVDIFNALDSGDLSD | |
| | DOMAIN IN | | FIEDVEWEVHGMPAVKNVISYGCCSEPYPDVTFTLLLKRRSH | |
| | COMPLEX WITH | | | |
| | ALPHA-CONOTOXIN | | | |
| | RGIA | | | |
| 41cy | CRYSTAL | HLA class I | GSHSMRYFYTAMSRPGRGEPRFIAVGYVDDTQFVRFDSDAAS | 92 |
| | STRUCTURE OF HLA- | histocompatibility | PRMAPRAPWIEQEGPEYWDRETQKYKRQAQTDRVSLRNLRG | |
| | B46 AT 1.6 | antigen, B-46 alpha | YYNQSEAGSHTLQRMYGCDVGPDGRLLRGHDQSAYDGKDYI | |
| | ANGSTROM | chain | ALNEDLSSWTAADTAAQITQRKWEAAREAEQWRAYLEGLCV | |
| | RESOLUTION | | EWLRRYLENGKETLQRADPPKTHVTHHPISDHEATLRCWALG | |
| | | | FYPAEITLTWQRDGEDQTQDTELVETRPAGDRTFQKWAAVV | |
| | | | VPSGEEQRYTCHVQHEGLPKPLTLRWXX | |
| 3hql | STRUCTURES OF | Speckle-type POZ | KVVKFSYMWTINNFSFCREEMGEVIKSSTFSSKLKWCLRVNP | 93 |
| | SPOP-SUBSTRATE | protein | KGLDEESKDYLSLYLLLVSCKSEVRAKFKFSILNAKGEETKA | |
| | COMPLEXES: | | MESQRAYRFVQGKDWGFKKFIRRGFLLDEANGLLPDDKLTLF | |
| | INSIGHTS INTO | | CEVSVV | |
| | MOLECULAR | | | |
| | ARCHITECTURES OF | | | |
| | BTB-CUL3 UBIQUITIN | | | |
| | LIGASES: SPOPMATH | | | |
| | X-PUCSBC1_PEP2 | | | |
| 3hqm | STRUCTURES OF | Speckle-type POZ | KVVKFSYMWTINNFSFCREEMGEVIKSSTFSSGDKLKWCLRV | 94 |
| | SPOP-SUBSTRATE | protein | NPKGLDEESKDYLSLYLLLVSCPSEVRAKFKFSILNAKGEETK | |
| | COMPLEXES: | | AMESQRAYRFVQGKDWGFKKFIRRGFLLDEANGLLPDDKLT | |
| | INSIGHTS INTO | | LFCEVSVVQ | |
| | MOLECULAR | | | |
| | ARCHITECTURES OF | | | |
| | BTB-CUL3 UBIQUITIN | | | |
| | LIGASES: | | | |
| | SPOPMATHX-CISBC2 | | | |
| 2ff6 | CRYSTAL | Gelsolin | EHPEFLKAGKEPGLQIWRVEKFDLVPVPTNLYGDFFTGDAYVI | 95 |
| | STRUCTURE OF | | LKTVQLRNGNLQYDLHYWLGNECSQDESGAAAIFTVQLDDY | |
| | GELSOLIN DOMAIN | | LNGRAVQHREVQGFESATFLGYFKSGLKYKKGGVASGFKHV | |
| | 1: CIBOULOT DOMAIN | | XX | |
| | 2 HYBRID IN | | | |
| | COMPLEX WITH | | | |
| | ACTIN | | | |
| 3f20 | CRYSTAL | SPRY domain- | DYCKPTRLDLLLDMPPVSYDVQLLHSWNNNDRSLNVFVKED | 96 |
| | STRUCTURE OF | containing SOCS | DKLIFHRHPVAQSTDAIRGKVGYTRGLHVWQITWAMRQRGT | |
| | HUMAN | box protein 1 | HAVVGVATADAPLHSVGYTTLVGNNHESWGWDLGRNRLYH | |
| | SPLA/RYANODINE | | DGKNQPSKTYPAFLEPDETFIVPDSFLVALDMDDGTLSFIVDG | |
| | RECEPTOR DOMAIN | | QYMGVAFRGLKGKKLYPVVSAVWGHCEIRMRYLNGLDPE | |
| | AND SOCS BOX | | | |
| | CONTAINING 1 | | | |
| | (SPSB1) IN COMPLEX | | | |
| | WITH A 20-RESIDUE | | | |
| | VASA PEPTIDE | | | |
| 2cia | HUMAN NCK2 SH2- | CYTOPLASMIC | SEWYYGNVTRHQAECALNERGVEGDFLIRDSESSPSDFSVSLK | 97 |
| | DOMAIN IN | PROTEIN NCK2 | ASGKNKHFKVQLVDNVYCIGQRRFHTMDELVEHYKKAPIFTS | |
| | COMPLEX WITH A | | EHGEKLYLVRALQXX | |
| | DECAPHOSPHOPEPTI | | | |
| | DE FROM | | | |
| | TRANSLOCATED | | | |
| | INTIMIN RECEPTOR | | | |
| | (TIR) OF EPEC | | | |
| 5h5q | CRYSTAL | Phospholipid | DDWRAARSMHEFSAKDIDGHMVNLDKYRGFVSIVTNVASQX | 99 |
| | STRUCTURE OF | hydroperoxide | GKTEVNYTQLVDLHARYAERGLRILAFPSNQFGKQEPGSNEEI | |
| | HUMAN GPX4 IN | glutathione | KEFAAGYNVKFDMFSKIEVNGDDAHPLWKWMKIQPKGKGIL | |
| | COMPLEX WITH | peroxidase, | GNAIKWNFTKFLIDKNGVVVKRYGPMEEPLVIEKDLPHYF | |
| | GXPEP-1 | mitochondrial | | |
| 1bt6 | P11 (S100A10), | S100A10 | PSQMEHAMETMMFTFHKFAGDKGYLTKEDLRVLMEKEFPGF | 100 |
| | LIGAND OF ANNEXIN | | LENQKDPLAVDKIMKDLDQCRDGKVGFQSFFSLIAGLTIACN | |
| | II IN COMPLEX WITH | | DYFVVHMK | |
| | ANNEXIN II N- | | | |
| | TERMINUS | | | |
| 6d3z | PROTEASE SFTI | Plasminogen | FDCGKPQVEPKKCPGVVGGCVAHPHSWPWQVSLRTRFGMHF | 101 |
| | COMPLEX | | CGGTLISPEWVLTAAHCLEKSPRPSSYKVILGAHQEVNLEPHV | |
| | | | QEIEVSRLFLEPTRKDIALLKLSSPAVITDKVIPACLPSPNYVVA | |
| | | | DRTECFITGWGETQGTFGAGLLKEAQLPVIENKVCNRYEFLN | |
| | | | GRVQSTELCAGHLAGGTDSCQGDAGGPLVCFEKDKYILQGVT | |
| | | | SWGLGCARPNKPGVYVRVSRFVTWIEGVMRNN | |
| 3iec | HELICOBACTER | Serine/threonine- | HIGNYRLLKTIGAKVKLARHILTGKEVAVKIIDKTQLNSSSLQ | 102 |
| | PYLORI CAGA | protein kinase | KLFREVRIMKVLNHPNIVKLFEVIETEKTLYLVMEYASGGEVF | |
| | INHIBITS PAR1/MARK | MARK2 | DYLVAHGWMKEKEARAKFRQIVSAVQYCHQKFIVHRDLKAE | |
| | FAMILY KINASES BY | | NLLLDADMNIKIADFGFSNEFTFGNKLDTFCGSPPYAAPELFQ | |
| | MIMICKING HOST | | GKKYDGPEVDVWSLGVILYTLVSGSLPFDGQNLKELRERVLR | |
| | SUBSTRATES | | GKYRIPFYMSTDCENLLKKFLILNPSKRGTLEQIMKDRWMNV | |
| | | | GHEDDELKPYVEPLPDYKDPRRTELMVSMGYTREEIQDSLVG | |
| | | | QRYNEVMATYLLLGY | |
| 3ua7 | CRYSTAL | Tyrosine-protein | GTGVTLFVALYDYEARTEDDLSFHKGEKFQILNSSEGDWWEA | 103 |
| | STRUCTURE OF THE | kinase Fyn | RSLTTGETGYIPSNYVAPVDSXX | |
| | HUMAN FYN SH3 | | | |
| | DOMAIN IN | | | |
| | COMPLEX WITH A | | | |
| | PEPTIDE FROM THE | | | |
| | HEPATITIS C VIRUS | | | |
| | NS5A-PROTEIN | | | |
| 4jlv | FUNCTIONAL AND | MOB kinase | LRQAVMLPEGEDLNEWIAVNTVDFFNQINMLYGTITEFCTEAS | 104 |
| | STRUCTURAL | activator 1A | CPVMSAGPRYEYHWADPIKCSAPKYIDYLMTWVQDQLDDET | |
| | STUDIES OF | | LFPSKIGVPFPKNFMSVAKTILKRLFRVYAHIYHQHFDSVMQL | |
| | MOBKLIB, A | | QEEAHLNTSFKHFIFFVQEFNLIDRRELAPLQELIEKLGX | |
| | SALVADOR/WARTS/ | | | |
| | HIPPO TUMOR | | | |
| | SUPPRESSOR | | | |
| | PATHWAY, IN HCV | | | |
| | REPLICATION | | | |
| 5jzi | CRYSTAL | HLA class I | GSHSMRYFFTSVSRPGRGEPRFIAVGYVDDTQFVRFDSDAASQ | 105 |
| | STRUCTURE OF 1406 | histocompatibility | RMEPRAPWIEQEGPEYWDGETRKVKAHSQTHRVDLGTLRGY | |
| | TCR BOUND TO HLA- | antigen, A-2 alpha | YNQSEAGSHTVQRMYGCDVGSDWRFLRGYHQYAYDGKDYI | |
| | A2 WITH HCV 1406- | chain | ALKEDLRSWTAADMAAQTTKHKWEAAHVAEQLRAYLEGTC | |
| | 1415 ANTIGEN | | VEWLRRYLENGKETLQRTDAPKTHMTHHAVDHEATLRCWA | |
| | PEPTIDE | | LSFYPAEILWQREDQTQDTEVETRPAGDGTFQKWAVVVPSGQ | |
| | | | EYTCHQHEGLPLTL | |
| 4jzo | THREE | Anti-HCV E2 Fab | SYVLTQPPSVSVSPGQTARITCSGDKLGDKYASWYQQKPGQS | 106 |
| | DIMENSIONAL | HC84-27 light | PVLVIYQDNKRPSEIPARFSGSNSGNTATLTISGAQAMDEADY | |
| | STRUCTURE OF | chain | YCQAWDSNTGVFGTGTKLTVLRTVAAPSVFIFPPSDEQLKSGT | |
| | BROADLY | | ASVVCLLNNFYPREAKVQWKVDNALQSGNSQESVTEQDSKD | |
| | NEUTRALIZING | | STYSLSSTLTLSKADYEKHKVYACEVTHQGLSSPVTKSFNRG | |
| | HUMAN ANTI - | | | |
| | HEPATITIS C VIRUS | | | |
| | (HCV) | | | |
| | GLYCOPROTEIN E2 | | | |
| | FAB FRAGMENT | | | |
| | HC84-27 | | | |
| 5kzp | STRUCTURE OF THE | HCV1-C1 | ELTLTQSPATLSLSPGERATLSCRASQSVSSYLAWYQQKPGQA | 107 |
| | HCV1-C1 ANTIBODY- | Antibody Fab | PRLLIYDASNRATGIPARFSGSGSGTDFTLTISSLEPEDFAVYYC | |
| | ANTIGEN COMPLEX | Light Chain | QQRSNWITFGQGTRLEIKRTVAAPSVFIFPPSDEQLKSGTASVV | |
| | | | CLLNNFYPREAKVQWKVDNALQSGNSQESVTEQDSKDSTYS | |
| | | | LSNTLTLSKADYEKHKVYACEVTHQGLSSPVTKSFNRGXXXX | |
| | | | X | |
| 3zhf | GAMMA 2 ADAPTIN | AP-1 COMPLEX | GSAPIPDLKVFEREGVQLNLSFIRPPENPALLLITITATNFSEGD | 108 |
| | EAR DOMAIN | SUBUNIT | VTHFICQAAVPKSLQLQLQAPSGNTVPARGGLPITQLFRILNPN | |
| | CRYSTAL | GAMMA-LIKE 2 | KAPLRLKLRLTYDHFHQSVQEIFEVNNLPVESWQX | |
| | STRUCTURE WITH | | | |
| | PRESI SITE1 PEPTIDE | | | |
| | NPDWDFN | | | |
| 1lhc | HUMAN ALPHA- | ALPHA- | IVEGSDAEIGMSPWQVMLFRKSPQELLCGASLISDRWVLTAA | 109 |
| | THROMBIN | THROMBIN | HCLLYPPWDKNFTENDLLVRIGKHSRTRYERNIEKISMLEKIYI | |
| | COMPLEXED WITH | | HPRYNWRENLDRDIALMKLKKPVAFSDYIHPVCLPDRETAAS | |
| | AC-(D)PHE-PRO- | | LLQAGYKGRVTGWGNLKEGQPSVLQVVNLPIVERPVCKDST | |
| | BOROARG-OH | | RIRITDNMFCAGYKPDEGKRGDACEGDSGGPFVMKSPFNNR | |
| | | | WYQMGIVSWGEGCDRDGKYGFYTHVFRLKKWIQKVIDQFX | |
| 3d49 | THROMBIN | Thrombin heavy | IVEGSDAEIGMSPWQVMLFRKSPQELLCGASLISDRWVLTAA | 110 |
| | INHIBITION | chain | HCLLYPPWDKNFTENDLLVRIGKHSRTRYERNIEKISMLEKIYI | |
| | | | HPRYNWRENLDRDIALMKLKKPVAFSDYIHPVCLPDRETAAS | |
| | | | LLQAGYKGRVTGWGNLKEGQPSVLQVVNLPIVERPVCKDST | |
| | | | RIRITDNMFCAGYKPDEGKRGDACEGDSGGPFVMKSPFNNR | |
| | | | WYQMGIVSWGEGCDRDGKYGFYTHVFRLKKWIQKVIDQFX | |
| | | | XX | |
| 1ai8 | HUMAN ALPHA- | ALPHA- | IVEGSDAEIGMSPWQVMLFRKSPQELLCGASLISDRWVLTAA | 111 |
| | THROMBIN TERNARY | THROMBIN | HCLLYPPWDKNFTENDLLVRIGKHSRTRYERNIEKISMLEKIYI | |
| | COMPLEX WITH THE | (LARGE | HPRYNWRENLDRDIALMKLKKPVAFSDYIHPVCLPDRETAAS | |
| | EXOSITE INHIBITOR | SUBUNIT) | LLQAGYKGRVTGWGNLKETKGQPSVLQVVNLPIVERPVCKD | |
| | HIRUGEN AND | | STRIRITDNMFCAGYKPDEGKRGDACEGDSGGPFVMKSPFNN | |
| | ACTIVE SITE | | RWYQMGIVSWGEGCDRDGKYGFYTHVFRLKKWIQKVIDQFX | |
| | INHIBITOR | | | |
| | PHCH2OCO-D-DPA- | | | |
| | PRO-BOROMPG | | | |
| 1hah | THE ISOMORPHOUS | ALPHA- | IVEGSDAEIGMSPWQVMLFRKSPQELLCGASLISDRWVLTAA | 112 |
| | STRUCTURES OF | THROMBIN | HCLLYPPWDKNFTENDLLVRIGKHSRTRYERNIEKISMLEKIYI | |
| | PRETHROMBIN2, | (LARGE | HPRYNWRENLDRDIALMKLKKPVAFSDYIHPVCLPDRETAAS | |
| | HIRUGEN-AND | SUBUNIT) | LLQAGYKGRVTGWGNLKETWGQPSVLQVVNLPIVERPVCKD | |
| | PPACK-THROMBIN: | | STRIRITDNMFCAGYKPDEGKRGDACEGDSGGPFVMKSPFNN | |
| | CHANGES | | RWYQMGIVSWGEGCDRDGKYGFYTHVFRLKKWIQKVIDQFG | |
| | ACCOMPANYING | | E | |
| | ACTIVATION AND | | | |
| | EXOSITE BINDING TO | | | |
| | THROMBIN | | | |
| 1hgt | STRUCTURE OF THE | ALPHA- | IVEGSDAEIGMSPWQVMLFRKSPQELLCGASLISDRWVLTAA | 113 |
| | HIRUGEN AND | THROMBIN | HCLLYPPWDKNFTENDLLVRIGKHSRTRYERNIEKISMLEKIYI | |
| | HIRULOG 1 | (LARGE | HPRYNWRENLDRDIALMKLKKPVAFSDYIHPVCLPDRETAAS | |
| | COMPLEXES OF | SUBUNIT) | LLQAGYKGRVTGWGNLKETWGQPSVLQVVNLPIVERPVCKD | |
| | ALPHA-THROMBIN | | STRIRITDNMFCAGYKPDEGKRGDACEGDSGGPFVMKSPFNN | |
| | | | RWYQMGIVSWGEGCDRDGKYGFYTHVFRLKKWIQKVIDQFG | |
| | | | E | |
| 1hxe | SERINE PROTEASE | THROMBIN | IVEGSDAEIGMSPWQVMLFRKSPQELLCGASLISDRWVLTAA | 114 |
| | | | HCLLYPPWDKNFTENDLLVRIGKHSRTRYERNIEKISMLEKIYI | |
| | | | HPRYNWRENLDRDIALMKLKKPVAFSDYIHPVCLPDRETAAS | |
| | | | LLQAGYKGRVTGWGNLKEGQPSVLQVVNLPIVERPVCKDST | |
| | | | RIRITDNMFCAGYKPDEGKRGDACEGDSGGPFVMKSPFNNR | |
| | | | WYQMGIVSWGEGCDRDGKYGFYTHVFRLKKWIQKVIDQXX | |
| 1zrb | THROMBIN IN | thrombin | DCGLRPLFEKKSLEDKTERELLESYIIVEGSDAEIGMSPWQVM | 115 |
| | COMPLEX WITH AN | | LFRKSPQELLCGASLISDRWVLTAAHCLLYPPWDKNFTENDL | |
| | AZAFLUORENYL | | LVRIGKHSRTRYERNIEKISMLEKIYIHPRYNWRENLDRDIAL | |
| | INHIBITOR 23B | | MKLKKPVAFSDYIHPVCLPDRETAASLLQAGYKGRVTGWGN | |
| | | | LKEGQPSVLQVVNLPIVERPVCKDSTRIRITDNMFCAGYKPDE | |
| | | | GKRGDACEGDSGGPFVMKSPFNNRWYQMGIVSWGEGCDRD | |
| | | | GKYGFYTHVFRLKKWIQKVIDQFGX | |
| 1mue | THROMBIN- | THROMBIN | IVEGSDAEIGMSPWQVMLFRKSPQELLCGASLISDRWVLTAA | 116 |
| | HIRUGEN-L405, 426 | | HCLLYPPWDKNFTENDLLVRIGKHSRTRYERNIEKISMLEKIYI | |
| | | | HPRYNWRENLDRDIALMKLKKPVAFSDYIHPVCLPDRETAAS | |
| | | | LLQAGYKGRVTGWGNLKEGQPSVLQVVNLPIVERPVCKDST | |
| | | | RIRITDNMFCAGYKPDEGKRGDACEGDSGGPFVMKSPFNNR | |
| | | | WYQMGIVSWGEGCDRDGKYGFYTHVFRLKKWIQKVIDQFG | |
| | | | X | |
| 1bhx | X-RAY STRUCTURE | ALPHA | IVEGSDAEIGMSPWQVMLFRKSPQELLCGASLISDRWVLTAA | 117 |
| | OF THE COMPLEX OF | THROMBIN | HCLLYPPWDKNFTENDLLVRIGKHSRTRYERNIEKISMLEKIYI | |
| | HUMAN ALPHA | | HPRYNWRENLDRDIALMKLKKPVAFSDYIHPVCLPDRETAAS | |
| | THROMBIN WITH THE | | LLQAGYKGRVTGWGNLKET | |
| | INHIBITOR SDZ 229- | | | |
| | 357 | | | |
| 1h9o | PHOSPHATIDYLINOSITOL | PHOSPHATIDYLI | GSPIPHHDEKTWNVGSSNRNKAENLLRGKRDGTFLVRESSKQ | 118 |
| | 3-KINASE, P85- | NOSITOL 3- | GCYACSVVVDGEVKHCVINKTATGYGFAEPYNLYSSLKELVL | |
| | ALPHA SUBUNIT: C- | KINASE | HYQHTSLVQHNDSLNVTLAYPVYAQ | |
| | TERMINAL SH2 | | | |
| | DOMAIN | | | |
| | COMPLEXED WITH A | | | |
| | TYR751 | | | |
| | PHOSPHOPEPTIDE | | | |
| | FROM THE PDGF | | | |
| | RECEPTOR, CRYSTAL | | | |
| | STRUCTURE AT 1.79 A | | | |
| 2aij | FORMYLGLYCINE | Sulfatase | LAHSKMVPIPAGVFTMGTDDPQIKQDGEAPARRVTIDAFYMD | 119 |
| | GENERATING | modifying factor 1 | AYEVSNTEFEKFVNSTGYLTEAEKFGDSFVFEGMLAAAPWW | |
| | ENZYME C336S | | LPVKGANWRHPEGPDSTILHRPDHPVLHVSWNDAVAYCTWA | |
| | MUTANT | | GKRLPTEAEWEYSCRGGLHNRLFPWGNKLQPKGQHYANIWQ | |
| | COVALENTLY | | GEFPVTNTGEDGFQGTAPVDAFPPNGYGLYNIVGNAWEWTS | |
| | BOUND TO | | DWWTVHHSVEETLNPKGPPSGKDRVKKGGSYMSHRSYCYR | |
| | SUBSTRATE PEPTIDE | | YRCAARSQNTPDSSASNLGFRCAADRLPXXX | |
| | CTPSR (SEQ ID NO: | | | |
| | 429) | | | |
| 2ce9 | A WRPW PEPTIDE | TRANSDUCIN- | DYFQGAMGSKPAYSFHVTADGQMQPVPFPPDALIGPGIPRHA | 120 |
| | BOUND TO THE | LIKE | RQINTLNHGEVVCAVTISNPTRHVYTGGKGCVKVWDISHPGN | |
| | GROUCHO-TLE WD40 | ENHANCER | KSPVSQLDCLNRDNYIRSCKLLPDGCTLIVGGEASTLSIWDLA | |
| | DOMAIN. | PROTEIN 1 | APTPRIKAELTSSAPACYALAISPDSKVCFSCCSDGNIAVWDLH | |
| | | | NQTLVRQFQGHTDGASCIDISNDGTKLWTGGLDNTVRSWDL | |
| | | | REGRQLQQHDFTSQIFSLGYCPTGEWLAVGMESSNVEVLHVN | |
| | | | KPDKYQLHLHESCVLSLKFAYCGKWFVSTGKDNLLNAWRTP | |
| | | | YGASIFQSKESSSVLSCDISVDDKYIVTGSGDKKATVYEVIY | |
| 2hi8 | HUMAN | Sulfatase- | LAHSKMVPIPAGVFTMGTDDPQIKQDGEAPARRVTIDAFYMD | 121 |
| | FORMYLGLYCINE | modifying factor 1 | AYEVSNTEFEKFVNSTGYLTEAEKFGDSFVFEGMLAAAPWW | |
| | GENERATING | | LPVKGANWRHPEGPDSTILHRPDHPVLHVSWNDAVAYCTWA | |
| | ENZYME, C336S | | GKRLPTEAEWEYSCRGGLHNRLFPWGNKLQPKGQHYANIWQ | |
| | MUTANT, BROMIDE | | GEFPVTNTGEDGFQGTAPVDAFPPNGYGLYNIVGNAWEWTS | |
| | CO- | | DWWTVHHSVEETLNPKGPPSGKDRVKKGGSYMSHRSYCYR | |
| | CRYSTALLIZATION | | YRCAARSQNTPDSSASNLGFRCAADRLPXXXXXXXXXXXXX | |
| | | | XXXXX | |
| 3cbo | SET7/9-ER-ADOHCY | Histone-lysine N- | HGVCWIYYPDGGSLVGEVNEDGEMTGEKIAYVYPDERTALY | 122 |
| | COMPLEX | methyltransferase | GKFIDGEMIEGKLATLMSTEEGRPHFELMPGNSVYHFDKSTSS | |
| | | SETD7 | CISTNALLPDPYESERVYVAESLISSAGEGLFSKVAVGPNTVM | |
| | | | SFYNGVRITHQEVDSRDWALNGNTLSLDEETVIDVPEPYNHV | |
| | | | SKYCASLGHKANHSFTPNCIYDMFVHPRFGPIKCIRTLRAVEA | |
| | | | DEELTVAYGYEAPEWYQVELKAFQATQX | |
| 3E50 | CRYSTAL | Insulin-degrading | NNPAIKRIGNHITKSPEDKREYRGLELANGIKVLLISDPTTDKS | 123 |
| | STRUCTURE OF | enzyme | SAALDVHIGSLSDPPNIAGLSHFCQHMLFLGTKKYPKENEYSQ | |
| | HUMAN INSULIN | | FLSEHAGSSNAFTSGEHTNYYFDVSHEHLEGALDRFAQFFLCP | |
| | DEGRADING ENZYME | | LFDESCKDREVNAVDSEHEKNVMNDAWRLFQLEKATGNPKH | |
| | IN COMPLEX WITH | | PFSKFGTGNKYTLETRPNQEGIDVRQELLKFHSAYYSSNLMA | |
| | TRANSFORMING | | VCVLGRESLDDLTNLVVKLFSEVENKNVPLPEFPEHPFQEEHL | |
| | GROWTH FACTOR- | | KQLYKIVPIKDIRNLYVTFPIPDLQKYYKSNPGHYLGHLIGHEG | |
| | ALPHA | | PGSLLSELKSKGWVNTLVGGQKEGARGFMFFIINVDLTEEGLL | |
| | | | HVEDIILHMFQYIQKLRAEGPQEWVFQECKDLNAVAFRFKDK | |
| | | | ERPRGYTSKIAGILHYYPLEEVLTAEYLLEEFRPDLIEMVLDKL | |
| | | | RPENVRVAIVSKSFEGKTDRTEEWYGTQYKQEAIPDEVIKKW | |
| | | | QNADLNGKFKLPTKNEFIPTNFEILPLEKEATPYPALIKDTAMS | |
| | | | KLWFKQDDKFFLPKACLNFEFFSPFAYVDPLHCNMAYLYLEL | |
| | | | LKDSLNEYAYAAELAGLSYDLQNTIYGMYLSVKGYNDKQPIL | |
| | | | LKKIIEKMATFEIDEKRFEIIKEAYMRSLNNFRAEQPHQHAMY | |
| | | | YLRLLMTEVAWTKDELKEALDDVTLPRLKAFIPQLLSRLHIEA | |
| | | | LLHGNITKQAALGIMQMVEDTLIEHAHTKPLLPSQLVRYREV | |
| | | | QLPDRGWFVYQQRNEVHNNCGIEIYYQTDMQSTSENMFLELF | |
| | | | CQIISEPCFNTLRTKEQLGYIVFSGPRRANGIQGLRFIIQSEKPPH | |
| | | | YLESRVEAFLITMEKSIEDMTEEAFQKHIQALAIRRLDKPKKLS | |
| | | | AECAKYWGEIISQQYNFDRDNTEVAYLKTLTKEDIIKFYKEML | |
| | | | AVDAPRRHKVSVHVLAREMDSCPVVGNLSQAPALPQPEVIQN | |
| | | | MTEFKRGLPLFPLVKPHX | |
| 3k2u | CRYSTAL | Hepatocyte growth | IIGGSSSLPGSHPWLAAIYIGDSFCAGSLVHTCWVVSAAHCFSH | 124 |
| | STRUCTURE OF HGFA | factor activator | SPPRDSVSVVLGQHFFNRTTDVTQTFGIEKYIPYTLYSVFNPSD | |
| | IN COMPLEX WITH | long chain | HDLVLIRLKKKGDRCATRSQFVQPICLPEPGSTFPAGHKCQIA | |
| | THE ALLOSTERIC | | GWGHLDVSGYSSSLREALVPLVADHKCSSPEVYGADISPNML | |
| | INHIBITORY | | CAGYFDCKSDACQGDSGGPLACEKNGVAYLYGIISWGCLHKP | |
| | ANTIBODY FAB40 | | GVYTRVANYVDWINDRIR | |
| 3o35 | CRYSTAL | Transcription | PNEDWCAVCQNGGELLCCEKCPKVFHLSCHVPTLTNFPSGEW | 125 |
| | STRUCTURE OF | intermediary factor | ICTFCRDLSKPEVEYDCDAEKKKTEGLVKLTPIDKRKCERLLL | |
| | TRIM24 PHD-BROMO | 1-alpha | FLYCHEMSLAFQDPVPLTVPDYYKIIKNPMDLSTIKKRLQEDY | |
| | COMPLEXED WITH | | SMYSKPEDFVADFRLIFQNCAEFNEPDSEVANAGIKLENYFEE | |
| | H3(23-31)K27AC | | LLKNLYPXX | |
| | PEPTIDE | | | |
| 3qj6 | THE CRYSTAL | Hepatoma-derived | PHAFKPGDLVFAKMKGYPHWPARIDDIAAVKPPPNKYPIFFFG | 126 |
| | STRUCTURE OF | growth factor- | THETAFLGPKDLFPYDKCKDKYGKPNKRKGFNEGLWEIQNNP | |
| | PWWP DOMAIN OF | related protein 2 | HASYSXX | |
| | HUMAN HEPATOMA- | | | |
| | DERIVED GROWTH | | | |
| | FACTOR 2 IN | | | |
| | COMPLEX WITH | | | |
| | H3K79ME3 PEPTIDE | | | |
| 3rbq | CO-CRYSTAL | Protein unc-119 | IGPEDVLGLQRITGDYLCSPEENIYKIDFVRFKIRDMDSGTVLF | 127 |
| | STRUCTURE OF | homolog A | EIKKPPNAGRFVRYQFTPAFLRLRQVGATVEFTVGDKPVNNF | |
| | HUMAN UNC119 | | RMIERHYFRNQLLKSFDFHFGFCIPSSKNTCEHIYDFPPLSEELI | |
| | (RETINA GENE 4) | | SEMIRHPYETQSDSFYFVDDRLVMHNKADYSYSG | |
| | AND AN N-TERMINAL | | | |
| | TRANSDUCIN-ALPHA | | | |
| | MIMICKING PEPTIDE | | | |
| 3rl7 | CRYSTAL | Disks large | MYEYEEITLERGNSGLGFSIAGGTDNPHIGDDSSIFITKIITGGA | 128 |
| | STRUCTURE OF | homolog 1 | AAQDGRLRVNDCILRVNEVDVRDVTHSKAVEALKEAGSIVRL | |
| | HDLG1-PDZ1 | | YVKRR | |
| | COMPLEXED WITH | | | |
| | APC | | | |
| 3vro | CRYSTAL | Signal transduction | EEARALGRAVRMLQRLEEQCVVSPPSLRDLLPRTAQLLREVA | 129 |
| | STRUCTURE OF THE | protein CBL-C | HSRRAGGPGGPGGSGDFLLIYLANLEAKSRQVAALLSRLRRQ | |
| | TYROSINE KINASE | | LAKLAIIFSHMHAELHALFPGGKYCGHMYQLTKAPAHTFWRE | |
| | BINDING DOMAIN OF | | SCGARCVLPWAEFESLLGTCHPVEPGCTALALRTTIDLTCSGH | |
| | CBL-C IN COMPLEX | | VSIFEFDVFTRLFQPWPTLLKNWQLLAVNHPGYMAFLTYDEV | |
| | WITH PHOSPHO-SRC | | QERLQACRDKPGSYIFRPSCTRLGQWAIGYVSSDGSILQTIPAN | |
| | PEPTIDE | | KPLSQVLLEGQKDGFYLYPDGKTHNPDLTX | |
| 3zdy | INTEGRIN ALPHAIIB | INTEGRIN | NICTTRGVSSCQQCLAVSPMCAWCSDEALPLGSPRCDLKENL | 130 |
| | BETA3 HEADPIECE | BETA-3 | LKDNCAPESIEFPVSEARVLEDRPLSDKGSGDSSQVTQVSPQRI | |
| | AND RGD PEPTIDE | | ALRLRPDDSKNFSIQVRQVEDYPVDIYYLMDLSYSMKDDLWS | |
| | COMPLEX | | IQNLGTKLATQMRKLTSNLRIGFGAFVDKPVSPYMYISPPEAL | |
| | | | ENPCYDMKTTCLPMFGYKHVLTLTDQVTRFNEEVKKQSVSR | |
| | | | NRDAPEGGFDAIMQATVCDEKIGWRNDASHLLVFTTDAKTHI | |
| | | | ALDGRLAGIVQPNDGQCHVGSDNHYSASTTMDYPSLGLMTE | |
| | | | KLSQKNINLIFAVTENVVNLYQNYSELIPGTTVGVLSMDSSNV | |
| | | | LQLIVDAYGKIRSKVELEVRDLPEELSLSFNATCLNNEVIPGLK | |
| | | | SCMGLKIGDTVSFSIEAKVRGCPQEKEKSFTIKPVGFKDSLIVQ | |
| | | | VTFDCDCACQAQAEPNSHRCNNGNGTFECGVCRCGPGWLGS | |
| | | | QCXXXXX | |
| 3zvy | PHD FINGER OF | E3 UBIQUITIN- | PSCKHCKDDVNRLCRVCACHLCGGRQDPDKQLMCDECDMA | 131 |
| | HUMAN UHRF1 IN | PROTEIN | FHIYCLDPPLSSVPSEDEWYCPECRXXXXX | |
| | COMPLEX WITH | LIGASE UHRF1 | | |
| | UNMODIFIED | | | |
| | HISTONE H3 N- | | | |
| | TERMINAL TAIL | | | |
| 4n3w | CRYSTAL | CREB-binding | KKIFKPEELRQALMPTLEALYRQDPESLPFRQPVDPQLLGIPDY | 132 |
| | STRUCTURE OF THE | protein | FDIVKNPMDLSTIKRKLDTGQYQEPWQYVDDVWLMFNNAW | |
| | BROMODOMAIN-PHD | | LYNRKTSRVYKFCSKLAEVFEQEIDPVMQSLGYCCGRKYEFS | |
| | FINGER MODULE OF | | PQTLCDDPSQPQTTKKKNDTLDPEPFVDCKECGRKMHQICVL | |
| | HUMAN | | HYDIIWPSGFVCDNCLXX | |
| | TRANSCRIPTIONAL | | | |
| | CO-ACTIVATOR CBP | | | |
| | IN COMPLEX WITH | | | |
| | ACETYLATED | | | |
| | HISTONE 4 PEPTIDE | | | |
| | (H4K20AC). | | | |
| 4ny3 | HUMAN PTPA IN | Serine/threonine- | NFIIPKKEIHTVPDMGKWKRSQAYADYIGFILTLNEGVKGKKL | 133 |
| | COMPLEX WITH | protein | TFEYRVSEAIEKLVALLNTLDRWIDETPPVDQPSRFGNKAYRT | |
| | PEPTIDE | phosphatase 2A | WYAKLDEEAENLVATVVPTHLAAAVPEVAVYLKESVGNSTR | |
| | | activator | IDYGTGHEAAFAAFLCCLCKIGVLRVDDQIAIVFKVFNRYLEV | |
| | | | MRKLQKTYRMEPAGGVWGLDDFQFLPFIWGSSQLIDHPYLEP | |
| | | | RHFVDEKAVNENHKDYMFLECILFITEMKTGPFAEHSNQLWN | |
| | | | ISAVPSWSKVNQGLIRMYKAECLEKFPVIQHFKFGSLLPIHPVT | |
| | | | S | |
| 4ztd | CRYSTAL | Proliferating cell | FEARLVQGSILKKVLEALKDLINEACWDISSSGVNLQSMDSSH | 134 |
| | STRUCTURE OF | nuclear antigen | VSLVQLTLRSEGFDTYRCDRNLAMGVNLTSMSKILKCAGNED | |
| | HUMAN PCNA IN | | IITLRAEDNADTLALVFEAPNQEKVSDYEMKLMDLDVEQLGIP | |
| | COMPLEX WITH A | | EQEYSCVVKMPSGEFARICRDLSHIGDAVVISCAKDGVKFSAS | |
| | TRAIP PEPTIDE | | GELGNGNIKLSQTSNVDKEEEAVTIEMNEPVQLTFALRYLNFF | |
| | | | TKATPLSSTVTLSMSADVPLVVEYKIADMGHLKYYLAP | |
| 5c27 | CRYSTAL | Tyrosine-protein | VYLDRKLLTLEDKELGSGNFGTVKKGYYQMKKVVKTVAVKI | 135 |
| | STRUCTURE OF SYK | kinase SYK | LKNEANDPALKDELLAEANVMQQLDNPYIVRMIGICEAESW | |
| | IN COMPLEX WITH | | MLVMEMAELGPLNKYLQQNRHVKDKNIIELVHQVSMGMKY | |
| | COMPOUND 2 | | LEESNFVHRDLAARNVLLVTQHYAKISDFGLSKALRADENXX | |
| | | | KAQTHGKWPVKWYAPECINYYKFSSKSDVWSFGVLMWEAF | |
| | | | SYGQKPYRGMKGSEVTAMLEKGERMGCPAGCPREMYDLMN | |
| | | | LCWTYDVENRPGFAAVELRLRNYYYDVVNHX | |
| 5ulo | CRYSTAL | 14-3-3 protein | MDKNELVQKAKLAEQAERYDDMAACMKSVTEQGAELSNEE | 136 |
| | STRUCTURE OF 14-3-3 | zeta/delta | RNLLSVAYKNVVGARRSSWRVVSSIEQKAEKKQQMAREYRE | |
| | ZETA IN COMPLEX | | KIETELRDICNDVLSLLEKFLIPNASQAESKVFYLKMKGDYYR | |
| | WITH A SERINE 124- | | YLAEVAADDKKGIVDQSQQAYQEAFEISKKEMQPTHPIRLGL | |
| | PHOSPHORYLATED | | ALNFSVFYYEILNSPEKACSLAKTAFDEAIAELDTLSEESYKDS | |
| | TBC1D7 PEPTIDE | | TLIMQLLRDNLTLWTXX | |
| 6bfj | CASPASE-3 MUTANT- | Caspase-3 | SGISLDNSYKMDYPEMGLCIIINNKNFHKSTGMTSRSGTDVDA | 137 |
| | T245D, S249D | | ANLRETFRNLKYEVRNKNDLTREEIVELMRDVSKEDHSKRSS | |
| | | | FVCVLLSHGEEGIIFGTNGPVDLKKITNFFRGDRCRSLTGKPKL | |
| | | | FIIQACRGTELDCGIETX | |
| 6dn6 | SPRY DOMAIN- | SPRY domain- | ENLYFQSMPEGLEELLSAPPPDLGAQRRHGWNPKDCSENIEV | 138 |
| | CONTAINING SOCS | containing SOCS | KEGGLYFERRPVAQSTDGARGKRGYSRGLHAWEISWPLEQR | |
| | BOX PROTEIN 2 | box protein 2 | GTHAVVGVATALAPLQTDHYAALLGSNSESWGWDIGRGKLY | |
| | COMPLEXED WITH | | HQSKGPGAPQYPEVPERLLVVLDMEEGTLGYAIGGTYLGPAF | |
| | INNN(ABU) (SEQ ID | | RGLKGRTLYPAVSAVWGQCQVRIRYLGE | |
| | NO: 448) CYCLIC | | | |
| | PEPTIDE INHIBITOR | | | |
| 6j8f | CRYSTAL | Tubulinyl-Tyr | DEATWERMWKHVAKIHPDGEKVAQRIRGATDLPKIPIPSVPTF | 139 |
| | STRUCTURE OF | carboxypeptidase 1 | QPSTPVPERLEAVQRYIRELQYNHTGTQFFEIKKSRPLTGLMD | |
| | SVBP-VASH1 WITH | | LAKEMTKEALPIKCLEAVILGIYLTNSMPTLERFPISFKTYFSG | |
| | PEPTIDE MIMIC THE | | NYFRHIVLGVNFAGRYGALGMSRREDLMYKPPAFRTLSELVL | |
| | C-TERMINAL OF | | DFEAAYGRCWHVLKKVKLGQSVSHDPHSVEQIEWKHSVLDV | |
| | ALPHA-TUBULIN | | ERLGRDDFRKELERHARDMRL | |
| 1csy | SYK TYROSINE | SYK PROTEIN | GSRRASVGSHEKMPWFHGKISREESEQIVLIGSKTNGKFLIRAR | 140 |
| | KINASE C-TERMINAL | TYROSINE | DNNGSYALCLLHEGKVLHYRIDKDKTGKLSIPEGKKFDTLWQ | |
| | SH2 DOMAIN | KINASE | LVEHYSYKADGLLRVLTVPCQKIGTQ | |
| | COMPLEXED WITH A | | | |
| | PHOSPHOPEPTIDEFROM | | | |
| | THE GAMMA | | | |
| | CHAIN OF THE HIGH | | | |
| | AFFINITY | | | |
| | IMMUNOGLOBIN G | | | |
| | RECEPTOR, NMR | | | |
| 1tw6 | STRUCTURE OF AN | Baculoviral IAP | GPAFPGMGSEELRLASFYDWPLTAEVPPELLAAAGFFHTGHQ | 141 |
| | ML-IAP/XIAP | repeat-containing | DKVRCFFCYGGLQSWKRGDDPWTEHAKWFPGCQFLLRSKGQ | |
| | CHIMERA BOUND TO | protein 7 | EYINNIHLTHSLXXX | |
| | A 9 MER PEPTIDE | | | |
| | DERIVED FROM | | | |
| | SMAC | | | |
| 2ain | SOLUTION | Afadin | MKEPEIITVTLKKQNGMGLSIVAAKGAGQDKLGIYVKSVVKG | 142 |
| | STRUCTURE OF THE | | GAADVDGRLAAGDQLLSVDGRSLVGLSQERAAELMTRTSSV | |
| | AF-6 PDZ DOMAIN | | VTLEVAKQGA | |
| | COMPLEXED WITH | | | |
| | THE C-TERMINAL | | | |
| | PEPTIDE FROM THE | | | |
| | BCR PROTEIN | | | |
| 2br9 | 14-3-3 PROTEIN | 14-3-3 PROTEIN | DREDLVYQAKLAEQAERYDEMVESMKKVAGMDVELTVEER | 143 |
| | EPSILON (HUMAN) | EPSILON | NLLSVAYKNVIGARRASWRIISSIEQKEENKGGEDKLKMIREY | |
| | COMPLEXED TO | | RQMVETELKLICCDILDVLDKHLIPAANTGESKVFYYKMKGD | |
| | PEPTIDE | | YHRYLAEFATGNDRKEAAENSLVAYKAASDIAMTELPPTHPI | |
| | | | RLGLALNFSVFYYEILNSPDRACRLAKAAFDDAIAELDTLSEE | |
| | | | SYKDSTLIMQLLRDNLTLWT | |
| 2eax | CRYSTAL | Peptidoglycan | CPGIVPRSVWGARETHCPRMTLPAKYGIIIHTAGRTCNISDECR | 144 |
| | STRUCTURE OF | recognition | LLVRDIQSFYIDRLKSCDIGYNFLVGQDGAIYEGVGWNVQGSS | |
| | HUMAN PGRP- | protein-I-beta | TPGYDDIALGITFMGTFTGIPPNAAALEAAQDLIQCAMVKGYL | |
| | IBETAC IN COMPLEX | | TPNYLLVGHSDVARTLSPGQALYNIISTWPHFKH | |
| | WITH GLYCOSAMYL | | | |
| | MURAMYL | | | |
| | PENTAPEPTIDE | | | |
| 2ri7 | CRYSTAL | Nucleosome- | TKLYCICKTPEDESKFYIGCDRCQNWYHGRCVGILQSEAELID | 145 |
| | STRUCTURE OF PHD | remodeling factor | EYVCPQCQSTEDAMTVLTPLTEKDYEGLKRVLRSLQAHKMA | |
| | FINGER-LINKER- | subunit BPTF | WPFLEPVDPNDAPDYYGVIKEPMDLATMEERVQRRYYEKLT | |
| | BROMODOMAIN Y17E | | EFVADMTKIFDNCRYYNPSDSPFYQCAEVLESFFVQKLKGFK | |
| | MUTANT FROM | | AXXX | |
| | HUMAN BPTF IN THE | | | |
| | H3(1-9)K4ME2 BOUND | | | |
| | STATE | | | |
| 3iqu | CRYSTAL | 14-3-3 protein | GAMGSMERASLIQKAKLAEQAERYEDMAAFMKGAVEKGEE | 146 |
| | STRUCTURE OF | sigma | LSXEERNLLSVAYKNVVGGQRAAWRVLSSIEQKSNEEGSEEK | |
| | HUMAN 14-3-3 SIGMA | | GPEVREYREKVETELQGVCDTVLGLLDSHLIKEAGDAESRVF | |
| | IN COMPLEX WITH | | YLKMKGDYYRYLAEVATGDDKKRIIDSARSAYQEAMDISKK | |
| | RAF1 PEPTIDE (6MER) | | EMPPTNPIRLGLALNFSVFHYEIANSPEEAISLAKTTFDEAMAD | |
| | | | LHTLSEDSYKDSTLIMQLLRDNLTLWTXXXX | |
| 3lny | SECOND PDZ | Tyrosine-protein | PKPGDIFEVELAKNDNSLGISVTGGVNTSVRHGGIYVKAVIPQ | 147 |
| | DOMAIN FROM | phosphatase non- | GAAESDGRIHKGDRVLAVNGVSLEGATHKQAVETLRNTGQV | |
| | HUMAN PTPIE IN | receptor type 13 | VHLLLEKGQSX | |
| | COMPLEX WITH RA- | | | |
| | GEF2 PEPTIDE | | | |
| 3u4s | HISTONE LYSINE | Lysine-specific | ETLNPSARIMTFYPTMEEFRNFSRYIAYIESQGAHRAGLAKVV | 148 |
| | DEMETHYLASE | demethylase 4A | PPKEWKPRASYDDIDDLVIPAPIQQLVTGQSGLFTQYNIQKKA | |
| | JMJD2A IN COMPLEX | | MTVREFRKIANSDKYCTPRYSEFEELERKYWKNLTFNPPIYGA | |
| | WITH T11C PEPTIDE | | DVNGTLYEKHVDEWNIGRLRTILDLVEKESGITIEGVNTPYLY | |
| | SUBSTRATE | | FGMWKTSFAWHTEDMDLYSINYLHFGEPKSWYSVPPEHGKR | |
| | CROSSLINKED TO N- | | LERLAKGFFPGSAQSCEAFLRHKMTLISPLMLKKYGIPFDKVT | |
| | OXALYL-D-CYSTEINE | | QEAGEFMITFPYGYHAGFNHGFNCAESTNFATRRWIEYGKQA | |
| | | | VLCSCRKDMVKISMDVFVRKFQPERYKLWKAGKDNTVIDHT | |
| | | | LPTPEAAEFLXXX | |
| 4bve | CRYSTAL | NAD- | GKLSLQDVAELIRARACQRVVVMVGAGISTPSGIPDFRSPGSG | 149 |
| | STRUCTURE OF | DEPENDENT | LYSNLQQYDLPYPEAIFELPFFFHNPKPFFTLAKELYPGNYKPN | |
| | HUMAN SIRT3 IN | PROTEIN | VTHYFLRLLHDKGLLLRLYTQNIDGLERVSGIPASKLVEAHGT | |
| | COMPLEX WITH | DEACETYLASE | FASATCTVCQRPFPGEDIRADVMADRVPRCPVCTGVVKPDIVF | |
| | THIOALKYLIMIDATE | SIRTUIN-3, | FGEPLPQRFLLHVVDFPMADLLLILGTSLEVEPFASLTEAVRSS | |
| | FORMED FROM THIO- | MITOCHONDRIAL | VPRLLINRDLVGPLAWHPRSRDVAQLGDVVHGVESLVELLG | |
| | ACETYL-LYSINE | | WTEEMRDLVQRETGKLXXX | |
| | ACS2-PEPTIDE | | | |
| 4fr3 | CRYSTAL | 14-3-3 protein | AMGSMERASLIQKAKLAEQAERYEDMAAFMKGAVEKGEELS | 150 |
| | STRUCTURE OF | sigma | XEERNLLSVAYKNVVGGQRAAWRVLSSIEQKSNKPEVREYRE | |
| | HUMAN 14-3-3 SIGMA | | KVETELQGVCDTVLGLLDSHLIKEAGDAESRVFYLKMKGDY | |
| | IN COMPLEX WITH | | YRYLAEVATGDDKKRIIDSARSAYQEAMDISKKEMPPTNPIRL | |
| | TASK-3 PEPTIDE AND | | GLALNFSVFHYEIANSPEEAISLAKTTFDEAMADLHTLSEDSY | |
| | STABILIZER 16-O-ME- | | KDSTLIMQLLRDNLTLWTXXX | |
| | FC-H | | | |
| 4hix | CRYSTAL | Humanized 3D6 | YVVMTQSPLSLPVTPGEPASISCKSSQSLLDSDGKTYLNWLLQ | 151 |
| | STRUCTURE OF A | Fab light chain | KPGQSPQRLIYLVSKLDSGVPDRFSGSGSGTDFTLKISRVEAED | |
| | HUMANISED 3D6 FAB | | VGVYYCWQGTHFPRTFGQGTKVEIKRTVAAPSVFIFPPSDEQL | |
| | BOUND TO AMYLOID | | KSGTASVVCLLNNFYPREAKVQWKVDNALQSGNSQESVTEQ | |
| | BETA PEPTIDE | | DSKDSTYSLSSTLTLSKADYEKHKVYACEVTHQGLSSPVTKSF | |
| | | | NRGEC | |
| 4pxm | THE ESTROGEN | Estrogen receptor | ALSLTADQMVSALLDAEPPILYSEYDPTRPFSEASMMGLLTNL | 153 |
| | RECEPTOR ALPHA | | ADRELVHMINWAKRVPGFVDLTLHDQVHLLECAWLEILMIG | |
| | LIGAND BINDING | | LVWRSMEHPGKLLFAPNLLLDRNQGKCVEGMVEIFDMLLAT | |
| | DOMAIN D538G | | SSRFRMMNLQGEEFVCLKSIILLNSGVYDHIHRVLDKITDTLIH | |
| | MUTANT IN | | LMAKAGLTLQQQHQRLAQLLLILSHIRHMSNKGMEHLYSMK | |
| | COMPLEX WITH | | CKNVVPLYGLLLEMLDAHRLX | |
| | ESTRADIOL AND A | | | |
| | GLUCOCORTICOID | | | |
| | RECEPTOR- | | | |
| | INTERACTING | | | |
| | PROTEIN 1 NR BOX II | | | |
| | PEPTIDE | | | |
| 4rh5 | CRYSTAL | Tyrosine-protein | DTLEGSMAQLKKGLESGTVLIQFEQLYRKKPGLAITFAKLPQN | 154 |
| | STRUCTURE OF | phosphatase non- | LDKNRYKDVLPYDTTRVLLQGNEDYINASYVNMEIPAANLV | |
| | PTPN3 (PTPH1) IN | receptor type 3 | NKYIATQGPLPHTCAQFWQVVWDQKLSLIVMLTTLTERGRTK | |
| | COMPLEX WITH | | CHQYWPDPPDVMNHGGFHIQCQSEDCTIAYVSREMLVTNTQ | |
| | EPS15 PTYR849 | | TGEEHTVTHLQYVAWPAHGVPDDSSDFLEFVNYVRSLRVDSE | |
| | PEPTIDE | | PVLVHSSAGIGRTGVLVTMETAMCLTERNLPIYPLDIVRKMRD | |
| | | | QRAMMVQTSSQYKFVCEAILRVYEEGLV | |
| 4wk2 | METAL ION AND | Integrin beta-1 | NRCLKANAKSCGECIQAGPNCGWCTNSTFLSARCDDLEALKK | 155 |
| | LIGAND BINDING OF | | KGCPPDDIENPRGSKDIKKNKNVTNKLKPEDITQIQPQQLVLR | |
| | INTEGRIN | | LRSGEPQTFTLKFKRAEDYPIDLYYLMDLSYSMKDDLENVKS | |
| | | | LGTDLMNEMRRITSDFRIGFGSFVEKTVMPYISTTPAKLRNPC | |
| | | | TSEQNCTTPFSYKNVLSLTNKGEVFNELVGKQRISGNLDSPEG | |
| | | | GFDAIMQVAVCGSLIGWRNVTRLLVFSTDAGFHFAGDGKLG | |
| | | | GIVLPNDGQCHLENNMYTMSHYYDYPSIAHLVQKLSENNIQTI | |
| | | | FAVTEEFQPVYKELKNLIPKSAVGTLSANSSNVIQLIIDAYNSL | |
| | | | SSEVILENGKLSEGVTISYKSYCKNGVNGTGENGRKCSNISIGD | |
| | | | EVQFEISITSNKCPKKDSDSFKIRPLGFTEEVEVILQYICECXXX | |
| 5eg2 | SET7/9 N265A IN | Histone-lysine N- | GVCWIYYPDGGSLVGEVNEDGEMTGEKIAYVYPDERTALYG | 156 |
| | COMPLEX WITH | methyltransferase | KFIDGEMIEGKLATLMSTEEGRPHFELMPGNSVYHFDKSTSSC | |
| | ADOHCY AND TAF10 | SETD7 | ISTNALLPDPYESERVYVAESLISSAGEGLFSKVAVGPNTVMSF | |
| | PEPTIDE | | YNGVRITHQEVDSRDWALNGATLSLDEETVIDVPEPYNHVSK | |
| | | | YCASLGHKANHSFTPNCIYDMFVHPRFGPIKCIRTLRAVEADE | |
| | | | ELTVAYGYDHSPPEAPEWYQVELKAFQATQXX | |
| 5f2u | STRUCTURE OF | Retinal rod | SAKDERAREILRGFKLNWMNLRDAETGKILWQGTEDLSVPGV | 157 |
| | FULLY MODIFIED | rhodopsin-sensitive | EHEARVPKKILKCKAVSRELNFSSTEQMEKFRLEQKVYFKGQ | |
| | FARNESYLATED | cGMP 3′,5′-cyclic | CLEEWFFEFGFVIPNSTNTWQSLIEAAPESQMMPASVLTGNVII | |
| | INPP5E PEPTIDE IN | phosphodiesterase | ETKFFDDDLLVSTSRVRLFYV | |
| | COMPLEX WITH | subunit delta | | |
| | PDE6D | | | |
| 5ggp | CRYSTAL | Protein O-linked- | VLDVEVYSSRSKVYVAVDGTTVLEDEAREQGRGIHVIVLNQA | 158 |
| | STRUCTURE OF N- | mannose beta-1,2- | TGHVMAKRVFDTYSPHEDEAMVLFLNMVAPGRVLICTVKDE | |
| | TERMINAL DOMAIN | N- | GSFHLKDTAKALLRSLGSQAGPALGWRDTWAFVGRKGGPVF | |
| | OF HUMAN PROTEIN | acetylglucosaminyl | GEKHSKSPALSSWGDPVLLKTDVPLSXX | |
| | O-MANNOSE BETA- | transferase 1 | | |
| | 1,2-N- | | | |
| | ACETYLGLUCOSAMI | | | |
| | NYLTRANSFERASE IN | | | |
| | COMPLEX WITH | | | |
| | GLCNAC- BETA1,2- | | | |
| | MAN-PEPTIDE | | | |
| 5lso | CRYSTAL | Splicing factor 45 | AMGKCPTKVVLLRNMVGAGEVDEDLEVETKEECEKYGKVG | 159 |
| | STRUCTURE OF SPF45 | | KCFIVEIPGAPDDEAVRIFLEFERVESAIKAVVDLNGRFGGRV | |
| | UHM DOMAIN WITH | | VKACFYNLDKFRVLDLAEQ | |
| | CYCLIC PEPTIDE | | | |
| | INHIBITOR | | | |
| 5xup | CRYSTAL | Telomeric repeat- | GLVAEAEAVAAGWMLDFLCLSLCRAFRDGRSEDFRRTRNSA | 160 |
| | STRUCTURE OF TRF1 | binding factor 1 | EAIIHGLSSLTACQLRTIYICQFLTRIAAGKTLDAQFENDERITP | |
| | AND TERB1 | | LESALMIWGSIEKEHDKLHEEIQNLIKIQAIAVCMENGNFKEA | |
| | | | EEVFERIFHMPFKSKLLMIISQKDTFHSFFQHFSYNHMMEKIKS | |
| | | | YVNYVLSEKSSTFLMKAAAKVVE | |
| 1ca9 | STRUCTURE OF TNF | PROTEIN (TNF | QDKIEALSSKVQQLERSIGLKDLAMADLEQKVLEMEASTYDG | 161 |
| | RECEPTOR | RECEPTOR | VFIWKISDFARKRQEAVAGRIPAIFSPAFYTSRYGYKMCLRIYL | |
| | ASSOCIATED | ASSOCIATED | NGDGTGRGTHLSLFFVVMKGPNDALLRWPFNQKVTLMLLDQ | |
| | FACTOR 2 IN | FACTOR 2) | NNREHVIDAFRPDVTSSSFQRPVNDMNIASGCPLFCPVSKMEA | |
| | COMPLEX WITH A | | KNSYVRDDAIFIKAIVDLTGL | |
| | PEPTIDE FROM TNF- | | | |
| | R2 | | | |
| 1qjb | 14-3-3 | 14-3-3 PROTEIN | MDKNELVQKAKLAEQAERYDDMAACMKSVTEQGAELSNEE | 162 |
| | ZETA/PHOSPHOPEPTIDE | ZETA/DELTA | RNLLSVAYKNVVGARRSSWRVVSSIEQKTEGAEKKQQMARE | |
| | COMPLEX (MODE | | YREKIETELRDICNDVLSLLEKFLIPNASQAESKVFYLKMKGD | |
| | 1) | | YYRYLAEVAAGDDKKGIVDQSQQAYQEAFEISKKEMQPTHPI | |
| | | | RLGLALNFSVFYYEILNSPEKACSLAKTAFDEAIAELDTLSEES | |
| | | | YKDSTLIMQLLRDNLTLWTSDT | |
| 1ujk | VHS DOMAIN OF | ADP-ribosylation | EPAMEPETLEARINRATNPLNKELDWASINGFCEQLNEDFEGP | 163 |
| | HUMAN GGA1 | factor binding | PLATRLLAHKIQSPQEWEAIQALTVLETCMKSCGKRFHDEVG | |
| | COMPLEXED WITH C- | protein GGA1 | KFRFLNELIKVVSPKYLGSRTSEKVKNKILELLYSWTVGLPEE | |
| | TERMINAL | | VKIAEAYQMLKKQGIVKXXX | |
| | PHOSPHOPEPTIDE | | | |
| | FROM BACE | | | |
| 3bun | CRYSTAL | E3 ubiquitin- | PGTVDKKMVEKCWKLMDKVVRLCQNPKLALKNSPPYILDLL | 164 |
| | STRUCTURE OF C- | protein ligase CBL | PDTYQHLRTILSRYEGKMETLGENEYFRVFMENLMKKTKQTI | |
| | CBL-TKB DOMAIN | | SLFKEGKERMYEENSQPRRNLTKLSLIFSHMLAELKGIFPSGLF | |
| | COMPLEXED WITH | | QGDTFRITKADAAEFWRKAFGEKTIVPWKSFRQALHEVHPISS | |
| | ITS BINDING MOTIF | | GLEAMALKSTIDLTCNDYISVFEFDIFTRLFQPWSSLLRNWNSL | |
| | IN SPROUTY4 | | AVTHPGYMAFLTYDEVKARLQKFIHKPGSYIFRLSCTRLGQW | |
| | | | AIGYVTADGNILQTIPHNKPLFQALIDGFREGFYLFPDGRNQNP | |
| | | | DLTG | |
| 3hqu | PHD2: FE: UN9: PARTIA | Egl nine homolog | LPALKLALEYIVPCMNKHGICVVDDFLGKETGQQIGDEVRAL | 165 |
| | L HIFI-ALPHA | 1 | HDTGKFTDGQLVSQKSDSSKDIRGDKITWIEGKEPGCETIGLL | |
| | SUBSTRATE | | MSSMDDLIRHCNGKLGSYKINGRTKAMVACYPGNGTGYVRH | |
| | COMPLEX | | VDNPNGDGRCVTCIYYLNKDWDAKVSGGILRIFPEGKAQFAD | |
| | | | IEPKFDRLLFFWSDRRNPHEVQPAYATRYAITVWYFDADERA | |
| | | | RAKVKXX | |
| 3qlc | COMPLEX | Transcriptional | GPLGSMGIVSCTACGQQVNHFQKDSIYRHPSLQVLICKNCFKY | 166 |
| | STRUCTURE OF ATRX | regulator ATRX | YMSDDISRDSDGMDEQCRWCAEGGNLICCDFCHNAFCKKCIL | |
| | ADD DOMAIN BOUND | | RNLGRRELSTIMDENNQWYCYICHPEPLLDLVTACNSVYENL | |
| | TO UNMODIFIED H3 | | XXX | |
| | 1-15 PEPTIDE | | | |
| 3shb | CRYSTAL | E3 ubiquitin- | SGPSCKHCKDDVNRLCRVCACHLCGGRQDPDKQLMCDECD | 167 |
| | STRUCTURE OF PHD | protein ligase | MAFHIYCLDPPLSSVPSEDEWYCPECRXXXX | |
| | DOMAIN OF UHRF1 | UHRF1 | | |
| 3sov | THE STRUCTURE OF | Low-density | APLLLYANRRDLRLVDATNGKENATIVVGGLEDAAAVDFVFS | 168 |
| | A BETA PROPELLER | lipoprotein | HGLIYWSDVSEEAIKRTEFNKTESVQNVVVSGLLSPDGLACD | |
| | DOMAIN IN | receptor-related | WLGEKLYWTDSETNRIEVSNLDGSLRKVLFWQELDQPRAIAL | |
| | COMPLEX WITH | protein 6 | DPSSGFMYWTDWGEVPKIERAGMDGSSRFIIINSEIYWPNGLT | |
| | PEPTIDE S | | LDYEEQKLYWADAKLNFIHKSNLDGTNRQAVVKGSLPHPFA | |
| | | | LTLFEDILYWTDWSTHSILACNKYTGEGLREIHSDIFSPMDIHA | |
| | | | FSQQRQPNATNPCGIDNGGCSHLCLMSPVKPFYQCACPTGVK | |
| | | | LLENGKTCKDXXXX | |
| 3v43 | CRYSTAL | Histone | HMEPIPICSFCLGTKEQNREKKPEELISCADCGNSGHPSCLKFS | 169 |
| | STRUCTURE OF MOZ | acetyltransferase | PELTVRVKALRWQCIECKTCSSCRDQGKNADNMLFCDSCDR | |
| | | KAT6A | GFHMECCDPPLTRMPKGMWICQICRPRXXXX | |
| 4es0 | X-RAY STRUCTURE | WD repeat- | VKPNYALKFTLAGHTKAVSSVKFSPNGEWLASSSADKLIKIW | 170 |
| | OF WDR5-SETDIB | containing protein | GAYDGKFEKTISGHKLGISDVAWSSDSNLLVSASDDKTLKIW | |
| | WIN MOTIF PEPTIDE | 5 | DVSSGKCLKTLKGHSNYVFCCNFNPQSNLIVSGSFDESVRIWD | |
| | BINARY COMPLEX | | VKTGKCLKTLPAHSDPVSAVHFNRDGSLIVSSSYDGLCRIWDT | |
| | | | ASGQCLKTLIDDDNPPVSFVKFSPNGKYILAATLDNTLKLWD | |
| | | | YSKGKCLKTYTGHKNEKYCIFANFSVTGGKWIVSGSEDNLVY | |
| | | | IWNLQTKEIVQKLQGHTDVVISTACHPTENIIASAALENDKTIK | |
| | | | LWKSDC | |
| 4l1u | CRYSTAL | RNA polymerase- | THMVSLPEELNRVRLSRHKLERWCHMPFFAKTVTGCFVRIGI | 171 |
| | STRUCTURE OF | associated protein | GNHNSKPVYRVAEITGVVETAKVYQLGGTRTNKGLQLRHGN | |
| | HUMAN RTF1 PLUS3 | RTF1 homolog | DQRVFRLEFVSNQEFTESEFMKWKEAMFSAGMQLPTLDEINK | |
| | DOMAIN IN | | KELSIKEAL | |
| | COMPLEX WITH SPT5 | | | |
| | CTR | | | |
| | PHOSPHOPEPTIDE | | | |
| 4l7x | CRYSTAL | Death-inducer | GPLPNALYCICRQPHNNRFMICCDRCEEWFHGDCVGISEARG | 172 |
| | STRUCTURE OF THE | obliterator 1 | RLLERNGEDYICPNCTXX | |
| | DIDO PHD FINGER IN | | | |
| | COMPLEX WITH | | | |
| | H3K4ME3 | | | |
| 4mvk | CRYSTAL | Neutrophil | LIPAPPLSKVPLQQNFQDNQFHGKWYVVGVAGNKSLREDKDP | 173 |
| | STRUCTURE OF AN | gelatinase- | WKMYATIYELKEDKSYNVTSVGFGTKKCHYKIRTFVPGSQPG | |
| | ENGINEERED | associated lipocalin | EFTLGRIKSRPGRTSALVRVVSTNYNQHAMVFFKVVQQNRES | |
| | LIPOCALIN | | FNITLYGRTKELTSELKENFIRFSKSLGLPENHIVFPVPIDQCID | |
| | (ANTICALIN US7) IN | | | |
| | COMPLEX WITH THE | | | |
| | ALZHEIMER | | | |
| | AMYLOID PEPTIDE | | | |
| | FRAGMENT VFFAED | | | |
| | (SEQ ID NO: 1006) | | | |
| 6jwm | CRYSTAL | SPRY domain- | LSCPEGLEELLSAPPPDLGAQRRHGWNPKDCSENIEVKEGGLY | 174 |
| | STRUCTURE OF THE | containing SOCS | FERRPVAQSTDGARGKRGYSRGLHAWEISWPLEQRGTHAVV | |
| | SPRY DOMAIN OF | box protein 2 | GVATALAPLQTDHYAALLGSNSESWGWDIGRGKLYHQSKGP | |
| | SPSB2 IN COMPLEX | | GAPQYPAGTQGEQLEVPERLLVVLDMEEGTLGYAIGGTYLGP | |
| | WITH CR7, A POTENT | | AFRGLKGRTLYPAVSAVWGQCQVRIRYLGERGSHHH | |
| | CYCLIC PEPTIDE | | | |
| | INHIBITOR OF SPSB2- | | | |
| | INOS INTERACTION | | | |
| 1gjb | ENGINEERING | UROKINASE- | IIGGEFTTIENQPWFAAIYRRHRGGSVTYVCGGSLMSPCWVIS | 175 |
| | INHIBITORS HIGHLY | TYPE | ATHCFIDYPKKEDYIVYLGRSRLNSNTQGEMKFEVENLILHKD | |
| | SELECTIVE FOR THE | PLASMINOGEN | YSADTLAHHNDIALLKIRSKEGRCAQPSRTIQTICLPSMYNDPQ | |
| | S1 SITES OF SER190 | ACTIVATOR | FGTSCEITGFGKEASTDYLYPEQLKMTVVKLISHRECQQPHYY | |
| | TRYPSIN-LIKE | | GSEVTTKMLCAADPQWKTDSCQGDSGGPLVCSLQGRMTLTG | |
| | SERINE PROTEASE | | IVSWGRGCALKDKPGVYTRVSHFLPWIRSHTKXXX | |
| | DRUG TARGETS | | | |
| 2blz | HUMAN ESTROGEN | Estrogen receptor | SLALSLTADQMVSALLDAEPPILYSEYDPTRPFSEASMMGLLT | 176 |
| | RECEPTOR ALPHA | | NLADRELVHMINWAKRVPGFVDLTLHDQVHLLEXAWLEILM | |
| | LIGAND-BINDING | | IGLVWRSMEHPGKLLFAPNLLLDRNQGKXVEGMVEIFDMLL | |
| | DOMAIN IN | | ATSSRFRMMNLQGEEFVCLKSIILLNSGVYTFEKDHIHRVLDKI | |
| | COMPLEX WITH | | TDTLIHLMAKAGLTLQQQHQRLAQLLLILSHIRHMSNKGMEH | |
| | 17METHYL-17ALPHA- | | LYSMKXKNVVPLSDLLLEMLDAHRX | |
| | DIHYDROEQUILENIN | | | |
| | AND A GLUCOC | | | |
| | INTERACTING | | | |
| | PROTEIN 1 NR BOX II | | | |
| | PEPTIDE | | | |
| 2kbs | SOLUTION | Harmonin | KEKKVFISLVGSRGLGCSISSGPIQKPGIFISHVKPGSLSAEVGL | 177 |
| | STRUCTURE OF | | EIGDQIVEVNGVDFSNLDHKEAVNVLKSSRSLTISIVAAAGRE | |
| | HARMONIN PDZ2 IN | | LFMT | |
| | COMPLEX WITH THE | | | |
| | CARBOXYL TAIL | | | |
| | PEPTIDE OF | | | |
| | CADHERIN23 | | | |
| 2rt5 | STRUCTURAL | Msx2-interacting | VDMVQLLKKYPIVWQGLLALKNDTAAVQLHFVSGNNVLAH | 178 |
| | INSIGHTS INTO THE | protein | RSLPLSEGGPPLRIAQRMRLEATQLEGVARRMTVETDYCLLL | |
| | RECRUITMENT OF | | ALPCGRDQEDVVSQTESLKAAFITYLQAKQAAGIINVPNPGSN | |
| | SMRT BY THE CO- | | QPAYVLQIFPPCEFSESHLSRLAPDLLASISNISPHLMIVIASV | |
| | REPRESSOR SHARP | | | |
| | UNDER | | | |
| | PHOSPHORYLATIVE | | | |
| | REGULATION | | | |
| 2w3o | CRYSTAL | BIFUNCTIONAL | GRLWLESPPGEAPPIFLPSDGQALVLGRGPLTQVTDRKCSRTQ | 179 |
| | STRUCTURE OF THE | POLYNUCLEOTI | VELVADPETRTVAVKQLGVNPSTTGTQELKPGLEGSLGVGDT | |
| | HUMAN PNKP FHA | DE | LYLVNGEHPLTLRWEXX | |
| | DOMAIN IN | PHOSPHATASE/ | | |
| | COMPLEX WITH AN | KINASE | | |
| | XRCC1-DERIVED | | | |
| | PHOSPHOPEPTIDE | | | |
| 3t03 | CRYSTAL | Peroxisome | DLRALAKHLYDSYIKSFPLTKAKARAILTGKTTDKSPFVIYDM | 180 |
| | STRUCTURE OF PPAR | proliferator- | NSLMMGEDKIKFKHIQEQSKEVAIRIFQGCQFRSVEAVQEITE | |
| | GAMMA LIGAND | activated receptor | YAKSIPGFVNLDLNDQVTLLKYGVHEIIYTMLASLMNKDGVLI | |
| | BINDING DOMAIN IN | gamma | SEGQGFMTREFLKSLRKPFGDFMEPKFEFAVKFNALELDDSDL | |
| | COMPLEX WITH A | | AIFIAVIILSGDRPGLLNVKPIEDIQDNLLQALELQLKLNHPESS | |
| | NOVEL PARTIAL | | QLFAKLLQKMTDLRQIVTEHVQLLQVIKKTETDMSLHPLLQEI | |
| | AGONIST GQ-16 | | YKDLYX | |
| 4bxf | 60S RIBOSOMAL | BIFUNCTIONAL | SALNFDSPSSLFESLISPIKTETFFKEFWEQKPLLIQRDDPALAT | 181 |
| | PROTEIN L27A | LYSINE- | YYGSLFKLTDLKSLCSRGMYYGRDVNVCRCVNGKKKVLNK | |
| | HISTIDINE | SPECIFIC | DGKAHFLQLRKDFDQKRATIQFHQPQRFKDELWRIQEKLECY | |
| | HYDROXYLASE | DEMETHYLASE | FGSLVGSNVYITPAGSQGLPPHYDDVEVFILQLEGEKHWRLY | |
| | (MINA53 Y209C) IN | AND HISTIDYL- | HPTVPLARECSVEAEERIGRPVHEFMLKPGDLLYFPRGTIHQA | |
| | COMPLEX WITH | HYDROXYLASE | DTPAGLAHSTHVTISTYQNNSWGDFLLDTISGLVFDTAKEDVE | |
| | MN(II), 2- | MINA | LRTGIPRQLLLQVESTTVATRRLSGFLRTLADRLEGTKELLSSD | |
| | OXOGLUTARATE | | MKKDFIMHRLPPYSAGDGAELSTPGGKLPRLDSVVRLQFKDH | |
| | (20G) AND 60S | | IVLTVLPQEKMVYIYHSLKNSRETHMMTEFHGLRFPLSHLDAL | |
| | RIBOSOMAL PROTEIN | | KQIWNSPAISVKDLKLTTDEEKESLVLSLWTECLIQVVXX | |
| | L27A (RPL27A G37C) | | | |
| | PEPTIDE FRAGMENT | | | |
| 4hrg | CRYSTAL | Protein S100-A10 | MPSQMEHAMETMMFTFHKFAGDKGYLTKEDLRVLMEKEFP | 182 |
| | STRUCTURE OF P11- | | GFLENQKDPLAVDKIMKDLDQCRDGKVGFQSFFSLIAGLTIAC | |
| | ANNEXIN A2(N- | | NDYFVVHMKQENLYFQGDSTVHEILSKLSLE | |
| | TERMINAL) FUSION | | | |
| | PROTEIN IN | | | |
| | COMPLEX WITH | | | |
| | AHNAKI PEPTIDE | | | |
| 4lxl | CRYSTAL | Lysine-specific | QNPSCKIMTFRPTMEEFKDFNKYVAYIESQGAHRAGLAKIIPP | 183 |
| | STRUCTURE OF | demethylase 4B | KEWKPRQTYDDIDDVVIPAPIQQVVTGQSGLFTQYNIQKKAM | |
| | JMJD2B COMPLEXED | | TVGEYRRLANSEKYCTPRHQDFDDLERKYWKNLTFVSPIYGA | |
| | WITH PYRIDINE-2,4- | | DISGSLYDDDVAQWNIGSLRTILDMVERECGTIIEGVNTPYLY | |
| | DICARBOXYLIC ACID | | FGMWKTTFAWHTEDMDLYSINYLHFGEPKSWYAIPPEHGKR | |
| | AND H3K9ME3 | | LERLAIGFFPGSSQGCDAFLRHKMTLISPIILKKYGIPFSRITQEA | |
| | | | GEFMITFPYGYHAGFNHGFNCAESTNFATLRWIDYGKVATQC | |
| | | | TCRKDMVKISMDVFVRILQPERYELWKQGKXXX | |
| 6e3g | STRUCTURE OF | Nuclear receptor | PYASLTEIEHLVQSVCKSYRETCQLRLEDLLRQRSNIFSREEVT | 184 |
| | RORGT IN COMPLEX | ROR-gamma | GYQRKSMWEMWERCAHHLTEAIQYVVEFAKRLSGFMELCQ | |
| | WITH A NOVEL | | NDQIVLLKAGAMEVVLVRMCRAYNADNRTVFFEGKYGGME | |
| | AGONIST. | | LFRALGCSELISSIFDFSHSLSALHFSEDEIALYTALVLINAHRP | |
| | | | GLQEKRKVEQLQYNLELAFHHHLCKTHRQSILAKLPPKGKLR | |
| | | | SLCSQHVERLQIFQHLHPIVVQAAFPPLYKELFSX | |
| 6uyz | CRYSTAL | Small ubiquitin- | EYIKLKVIGQDSSEIHFKVKMTTHLKXLKESYAQRQGVPMNS | 185 |
| | STRUCTURE OF K46- | related modifier 1 | LRFLFEGQRIADNHTPKELGMEEEDVIEVYQEQTG | |
| | ACETYLATED SUMO1 | | | |
| | IN COMPLEX WITH | | | |
| | PHOSPHORYLATED | | | |
| | DAXX | | | |
| 1gwq | HUMAN OESTROGEN | OESTROGEN | SKKNSLALSLTADQMVSALLDAEPPILYSEPFSEASMMGLLTN | 186 |
| | RECEPTOR ALPHA | RECEPTOR | LADRELVHMINWAKRVPGFVDLTLHDQVHLLECAWLEILMI | |
| | LIGAND-BINDING | | GLVWRSMEHPGKLLFAPNLLLDRNQGKCVEGMVEIFDMLLA | |
| | DOMAIN IN | | TSSRFRMMNLQGEEFVCLKSIILLNSGVYTFLSSTLKSLEEKDH | |
| | COMPLEX WITH | | IHRVLDKITDTLIHLMAKAGLTLQQQHQRLAQLLLILSHIRHM | |
| | RALOXIFENE CORE | | SNKGMEHLYSMKCKNVVPLYDLLLEMLDAHRX | |
| | AND TIF2 NRBOX2 | | | |
| | PEPTIDE | | | |
| 1lck | SH3-SH2 DOMAIN | P56==LCK== | DNLVIALHSYEPSHDGDLGFEKGEQLRILEQSGEWWKAQSLT | 187 |
| | FRAGMENT OF | TYROSINE | TGQEGFIPFNFVAKANSLEPEPWFFKNLSRKDAERQLLAPGNT | |
| | HUMAN P56-LCK | KINASE | HGSFLIRESESTAGSFSLSVRDFDQNQGEVVKHYKIRNLDNGG | |
| | TYROSINE KINASE | | FYISPRITFPGLHELVRHYTNASDGLCTRLSRPCQT | |
| | COMPLEXED WITH | | | |
| | THE 10 RESIDUE | | | |
| | SYNTHETIC | | | |
| | PHOSPHOTYROSYL | | | |
| | PEPTIDE | | | |
| | TEGQPYQPQPA (SEQ | | | |
| | ID NO: 1007) | | | |
| 1oga | A STRUCTURAL | T-CELL | GITQSPKYLFRKEGQNVTLSCEQNLNHDAMYWYRQDPGQGL | 188 |
| | BASIS FOR | RECEPTOR | RLIYYSQIVNDFQKGDIAEGYSVSREKKESFPLTVTSAQKNPT | |
| | IMMUNODOMINANT | BETA CHAIN C | AFYLCASSSRSSYEQYFGPGTRLTVTEDLKNVFPPEVAVFEPSE | |
| | HUMAN T-CELL | REGION | AEISHTQKATLVCLATGFYPDHVELSWWVNGKEVHSGVSTDP | |
| | RECEPTOR | | QPLKEQPALNDSRYSLSSRLRVSATFWQNPRNHFRCQVQFYG | |
| | RECOGNITION. | | LSENDEWTQDRAKPVTQIVSAEAWGRADQ | |
| 1qew | HUMAN CLASS I | PROTEIN (HLA | GSHSMRYFFTSVSRPGRGEPRFIAVGYVDDTQFVRFDSDAASQ | 189 |
| | HISTOCOMPATIBILITY | CLASS I | RMEPRAPWIEQEGPEYWDGETRKVKAHSQTHRVDLGTLRGY | |
| | ANTIGEN (HLA-A | HISTOCOMPATI | YNQSEAGSHTVQRMYGCDVGSDWRFLRGYHQYAYDGKDYI | |
| | 0201) COMPLEX WITH | BILITY | ALKEDLRSWTAADMAAQTTKHKWEAAHVAEQLRAYLEGTC | |
| | A NONAMERIC | ANTIGEN, B-35 | VEWLRRYLENGKETLQRTDAPKTHMTHHAVSDHEATLRCW | |
| | PEPTIDE FROM | B* 3501 ALPHA | ALSFYPAEITLTWQRDGEDQTQDTELVETRPAGDGTFQKWAA | |
| | MELANOMA- | CHAIN) | VVVPSGQEQRYTCHVQHEGLPKPLTLRWE | |
| | ASSOCIATED | | | |
| | ANTIGEN 3 | | | |
| | (RESIDUES 271- 279) | | | |
| 1xr9 | CRYSTAL | HLA class I | GSHSMRYFYTAMSRPGRGEPRFIAVGYVDDTQFVRFDSDAAS | 190 |
| | STRUCTURES OF | histocompatibility | PRMAPRAPWIEQEGPEYWDRETQISKTNTQTYRESLRNLRGY | |
| | HLA-B*1501 IN | antigen, B-15 alpha | YNQSEAGSHTLQRMYGCDVGPDGRLLRGHDQSAYDGKDYIA | |
| | COMPLEX WITH | chain | LNEDLSSWTAADTAAQITQRKWEAAREAEQWRAYLEGLCVE | |
| | PEPTIDES FROM | | WLRRYLENGKETLQRADPPKTHVTHHPISDHEATLRCWALGF | |
| | HUMAN UBCH6 AND | | YPAEITLTWQRDGEDQTQDTELVETRPAGDRTFQKWAAVVV | |
| | EPSTEIN-BARR VIRUS | | PSGEEQRYTCHVQHEGLPKPLTLRWEPX | |
| | EBNA-3 | | | |
| 2cbl | N-TERMINAL | PROTO- | PPGTVDKKMVEKCWKLMDKVVRLCQNPKLALKNSPPYILDL | 191 |
| | DOMAIN OF CBL IN | ONCOGENE CBL | LPDTYQHLRTILSRYEGKMETLGENEYFRVFMENLMKKTKQT | |
| | COMPLEX WITH ITS | | ISLFKEGKERMYEENSQPRRNLTKLSLIFSHMLAELKGIFPSGL | |
| | BINDING SITE ON | | FQGDTFRITKADAAEFWRKAFGEKTIVPWKSFRQALHEVHPIS | |
| | ZAP-70 | | SGLEAMALKSTIDLTCNDYISVFEFDIFTRLFQPWSSLLRNWNS | |
| | | | LAVTHPGYMAFLTYDEVKARLQKFIHKPGSYIFRLSCTRLGQ | |
| | | | WAIGYVTADGNILQTIPHNKPLFQALIDGFREGFYLFPDGRNQ | |
| | | | NPDLTGX | |
| 2dlx | THE CRYSTAL | cortactin isoform a | NDLGITAVALYDYQAAGDDEISFDPDDIITNIEMIDDGWWRG | 192 |
| | STRUCTURE OF THE | | VCKGRYGLFPANYVELRQ | |
| | CORTACTIN-SH3 | | | |
| | DOMAIN AND | | | |
| | AMAP1-PEPTIDE | | | |
| | COMPLEX | | | |
| 2yln | STRUCTURE OF C- | E3 UBIQUITIN- | DKKMVEKCWKLMDKVVRLCQNPKLALKNSPPYILDLLPDTY | 193 |
| | CBL-ZAP-70 PEPTIDE | PROTEIN | QHLRTILSRYEGKMETLGENEYFRVFMENLMKKTKQTISLFK | |
| | COMPLEX | LIGASE | EGKERMYEENSQPRRNLTKLSLIFSHMLAELKGIFPSGLFQGD | |
| | | | TFRITKADAAEFWRKAFGEKTIVPWKSFRQALHEVHPISSGLE | |
| | | | AMALKSTIDLTCNDYISVFEFDIFTRLFQPWSSLLRNWNSLAV | |
| | | | THPGYMAFLTYDEVKARLQKFIHKPGSYIFRLSCTRLGQWAIG | |
| | | | YVTADGNILQTIPHNKPLFQALIDGFREGFYLFPDGRNONPDL | |
| | | | TGLCEKVTQEQYELYCEMGSTFQLCKICAENDKDVKIEPCGH | |
| | | | LMCTSCLTSWQESEGQGCPFCRCEIKGTEPIVVDPFDXXX | |
| 3al3 | CRYSTAL | DNA | KQYIFQLSSLNPQERIDYCHLIEKLGGLVIEKQCFDPTCTHIVV | 194 |
| | STRUCTURE OF | topoisomerase 2- | GHPLRNEKYLASVAAGKWVLHRSYLEACRTAGHFVQEEDYE | |
| | TOPBP1 BRCT7/8- | binding protein 1 | WGSSSILDVLTGINVQQRRLALAAMRWRKKIQQRQESGIVEG | |
| | BACH1 PEPTIDE | | AFSGWKVILHVDQSREAGFKRLLQSGGAKVLPGHSVPLFKEA | |
| | COMPLEX | | THLFSDLGVNIAEAAAQNVYCLRTEYIADYLMQESPPHVENY | |
| | | | CLPEAIS | |
| 3uu7 | CRYSTAL | Estrogen receptor | LALSLTADQMVSALLDAEPPILYSEYDPTRPFSEASMMGLLTN | 195 |
| | STRUCTURE OF | | LADRELVHMINWAKRVPGFVDLTLHDQVHLLEXAWLEILMI | |
| | HERA-LBD (Y537S) IN | | GLVWRSMEHPGKLLFAPNLLLDRNQGKXVEGMVEIFDMLLA | |
| | COMPLEX WITH | | TSSRFRMMNLQGEEFVCLKSIILLNSGVYTFKSLEEKDHIHRVL | |
| | BISPHENOL-A | | DKITDTLIHLMAKAGLTLQQQHQRLAQLLLILSHIRHMSNKG | |
| | | | MEHLYSMKCKNVVPLSDLLLEMLDAHRLX | |
| 3uzd | CRYSTAL | 14-3-3 protein | VDREQLVQKARLAEQAERYDDMAAAMKNVTELNEPLSNEER | 196 |
| | STRUCTURE OF 14-3-3 | gamma | NLLSVAYKNVVGARRSSWRVISSIEQKTSNEKIEMVRAYREKI | |
| | GAMMA | | EKELEAVCQDVLSLLDNYLIKNCSETQYESKVFYLKMKGDYY | |
| | | | RYLAEVATGEKRATVVESSEKAYSEAHEISKEHMQPTHPIRLG | |
| | | | LALNYSVFYYEIQNAPEQACHLAKTAFDDAIAELDTLNEDSY | |
| | | | KDSTLIMQLLRDNLTLWTX | |
| 4ch9 | CRYSTAL | KELCH-LIKE | PKVMIVVGGQAPKAIRSVECYDFEEDRWDQIAELPSRRCRAG | 197 |
| | STRUCTURE OF THE | PROTEIN 3 | VVFMAGHVYAVGGFNGSLRVRTVDVYDGVKDQWTSIASMQ | |
| | HUMAN KLHL3 | | ERRSTLGAAVLNDLLYAVGGFDGSTGLASVEAYSYKTNEWFF | |
| | KELCH DOMAIN IN | | VAPMNTRRSSVGVGVVEGKLYAVGGYDGASRQCLSTVEQYN | |
| | COMPLEX WITH A | | PATNEWIYVADMSTRRSGAGVGVLSGQLYATGGHDGPLVRK | |
| | WNK4 PEPTIDE | | SVEVYDPGTNTWKQVADMNMCRRNAGVCAVNGLLYVVGG | |
| | | | DDGSCNLASVEYYNPVTDKWTLLPTNMSTGRSYAGVAVIHK | |
| | | | X | |
| 4jyi | CRYSTAL | Retinoic acid | SYEMTAELDDLTEKIRKAHQETFPSLCQLGKYTTNSSADHRV | 198 |
| | STRUCTURE OF | receptor beta | RLDLGLWDKFSELATKCIIKIVEFAKRLPGFTGLTIADQITLLK | |
| | RARBETA LBD IN | | AACLDILILRICTRYTPEQDTMTFSDGLTLNRTQMHNAGFGPL | |
| | COMPLEX WITH | | TDLVFTFANQLLPLEMDDTETGLLSAICLICGDRQDLEEPTKV | |
| | SELECTIVE PARTIAL | | DKLQEPLLEALKIYIRKRRPSKPHMFPKILMKITDLRSISAKGA | |
| | AGONIST BMS641 [3- | | ERVITLKMEIPGSMPPLIQEMMEX | |
| | CHLORO-4-[I-2-(5,5- | | | |
| | DIMETHYL-8- | | | |
| | PHENYL-5,6- | | | |
| | DIHYDRONAPHTHALEN- | | | |
| | 2-YL) | | | |
| | ETHENYL]BENZOIC | | | |
| | ACID] | | | |
| 4nnx | CRYSTAL | HLA class I | GSHSMRYFFTSVSRPGRGEPRFIAVGYVDDTQFVRFDSDAASQ | 199 |
| | STRUCTURE OF PKD2 | histocompatibility | RMEPRAPWIEQEGPEYWDGETRKVKAHSQTHRVDLGTLRGY | |
| | PHOSPHOPEPTIDE | antigen, A-2 alpha | YNQSEAGSHTVQRMYGCDVGSDWRFLRGYHQYAYDGKDYI | |
| | BOUND TO HLA-A2 | chain | ALKEDLRSWTAADMAAQTTKHKWEAAHVAEQLRAYLEGTC | |
| | | | VEWLRRYLENGKETLQRTDAPKTHMTHHAVSDHEATLRCW | |
| | | | ALSFYPAEITLTWQRDGEDQTQDTELVETRPAGDGTFQKWAA | |
| | | | VVVPSGQEQRYTCHVQHEGLPKPLTLRWXXX | |
| 5E50 | APLF/XRCC4 | Aprataxin and | GPLGSMSGGFELQPRDGGPRVALAPGETVIGRGPLLGITDKRV | 200 |
| | COMPLEX | PNK-like factor | SRRHAILEVAGGQLRIKPIHTNPCFYQSSEKSQLLPLKPNLWCY | |
| | | | LNPGDSFSMLVDKYIFRILSIPS | |
| 6ivx | DISCOVERY OF THE | Nuclear receptor | SLTEIEHLVQSVCKSYRETCQLRLEDLLRQRSNIFSREEVTGYQ | 201 |
| | SECOND | ROR-gamma | RKSMWEMWERCAHHLTEAIQYVVEFAKRLSGFMELCQNDQI | |
| | GENERATION ROR | | VLLKAGAMEVVLVRMCRAYNADNRTVFFEGKYGGMELFRA | |
| | GAMMA INHIBITORS | | LGCSELISSIFDFSHSLSALHFSEDEIALYTALVLINAHRPGLQE | |
| | COMPOSED OF AN | | KRKVEQLQYNLELAFHHHLCKTHRQSILAKLPPAGKLASLCS | |
| | AZOLE SCAFFOLD. | | QHVERLQIFQHLHPIX | |
| 1mw4 | SOLUTION | Growth factor | GSPASGTSLSAAIHRTQLWFHGRISREESQRLIGQQGLVDGLFL | 202 |
| | STRUCTURE OF THE | receptor-bound | VRESQRNPQGFVLSLCHLQKVKHYLILPSEEEGRLYFSMDDG | |
| | HUMAN GRB7-SH2 | protein 7 | QTRFTDLLQLVEFHQLNRGILPCLLRHCCTRVAL | |
| | DOMAIN IN | | | |
| | COMPLEX WITH A 10 | | | |
| | AMINO ACID PEPTIDE | | | |
| | PY1139 | | | |
| 1om9 | STRUCTURE OF THE | ADP-ribosylation | ASITVPLESIKPSNILPVTVYDQHGFRILFHFARDPLPGRSDVLV | 203 |
| | GGA1-APPENDAGE IN | factor binding | VVVSMLSTAPQPIRNIVFQSAVPKVMKVKLQPPSGTELPAFNPI | |
| | COMPLEX WITH THE | protein GGA1 | VHPSAITQVLLLANPQKEKVRLRYKLTFTMGDQTYNEMGDV | |
| | P56 BINDING PEPTIDE | | DQFPPPETWGSL | |
| 209v | THE SECOND SH3 | Ponsin | GIDPFTGEAIAKFNFNGDTQVEMSFRKGERITLLRQVDENWYE | 204 |
| | DOMAIN FROM | | GRIPGTSRQGIFPITYVDVIKRPL | |
| | PONSIN IN COMPLEX | | | |
| | WITH THE PAXILLIN | | | |
| | PROLINE RICH | | | |
| | REGION | | | |
| 2q7i | THE WILD TYPE | Androgen receptor | PIFLNVLEAIEPGVVCAGHDNNQPDSFAALLSSLNELGERQLV | 205 |
| | ANDROGEN | | HVVKWAKALPGFRNLHVDDQMAVIQYSWMGLMVFAMGWR | |
| | RECEPTOR LIGAND | | SFTNVNSRMLYFAPDLVFNEYRMHKSRMYSQCVRMRHLSQE | |
| | BINDING DOMAIN | | FGWLQITPQEFLCMKALLLFSIIPVDGLKNQKFFDELRMNYIK | |
| | BOUND WITH | | ELDRIISCSRRFYQLTKLLDSVQPIARELHQFTFDLLIKSHMVS | |
| | TESTOSTERONE AND | | VDFPEMMAEIISVQVPKILSGKVKPIYFHTQX | |
| | AN AR 20-30 PEPTIDE | | | |
| 2r01 | SHORT FORM HGFA | Hepatocyte growth | IIGGSSSLPGSHPWLAAIYIGDSFCAGSLVHTCWVVSAAHCFSH | 206 |
| | WITH INHIBITORY | factor activator | SPPRDSVSVVLGQHFFNRTTDVTQTFGIEKYIPYTLYSVFNPSD | |
| | FAB75 | | HDLVLIRLKKKGDRCATRSQFVQPICLPEPGSTFPAGHKCQIA | |
| | | | GWGHLDENVSGYSSSLREALVPLVADHKCSSPEVYGADISPN | |
| | | | MLCAGYFDCKSDACQGDSGGPLACEKNGVAYLYGIISWGDG | |
| | | | CGRLHKPGVYTRVANYVDWINDRIRX | |
| 2rr4 | COMPLEX | Zinc finger CW- | GSSGSSGEISGFGQCLVWVQCSFPNCGKWRRLCGNIDPSVLPD | 207 |
| | STRUCTURE OF THE | type PWWP | NWSCDQNTDVQYNRCDIPEETWTGLEX | |
| | ZF-CW DOMAIN AND | domain protein 1 | | |
| | THE H3K4ME3 | | | |
| | PEPTIDE | | | |
| 3oll | CRYSTAL | Estrogen receptor | ALSPEQLVLTLLEAEPPHVLISRPSAPFTEASMMMSLTKLADK | 208 |
| | STRUCTURE OF | beta | ELVHMISWAKKIPGFVELSLFDQVRLLESCWMEVLMMGLMW | |
| | PHOSPHORYLATED | | RSIDHPGKLIFAPDLVLDRDEGKCVEGILEIFDMLLATTSRFRE | |
| | ESTROGEN | | LKLQHKEYLCVKAMILLNSSMYPLVTATADSSRKLAHLLNAV | |
| | RECEPTOR BETA | | TDALVWVIAKSGISSQQQSMRLANLLMLLSHVRHASNKGME | |
| | LIGAND BINDING | | HLLNMKCKNVVPVXDLLLEMLNAHX | |
| | DOMAIN | | | |
| 3pfv | CRYSTAL | E3 ubiquitin- | RTVEKTWKLMDKVVRLCQNPKLQLKNSPPYILDILPDTYQHL | 209 |
| | STRUCTURE OF CBL- | protein ligase | RLILSKYDDNQKLAQLSENEYFKIYIDSLMKKSKRAIRLFKEG | |
| | B TKB DOMAIN IN | CBL-B | KERMYEEQSQDRRNLTKLSLIFSHMLAEIKAIFPNGQFQGDNF | |
| | COMPLEX WITH | | RITKADAAEFWRKFFGDKTIVPWKVFRQCLHEVHQISSGLEA | |
| | EGFR PY1069 PEPTIDE | | MALKSTIDLTCNDYISVFEFDIFTRLFQPWGSILRNWNFLAVTH | |
| | | | PGYMAFLTYDEVKARLQKYSTKPGSYIFRLSCTRLGQWAIGY | |
| | | | VTGDGNILQTIPHNKPLFQALIDGSREGFYLYPDGRSYNPDLT | |
| | | | GLAXXX | |
| 3qhe | CRYSTAL | Adenomatous | RREIRVLHLLEQIRAYCETCWEWQEAHNPMPAPVEHQICPAV | 210 |
| | STRUCTURE OF THE | polyposis coli | CVLMKLSFDEEHRHAMNELGGLQAIAELLQVDCEMYGLTND | |
| | COMPLEX BETWEEN | protein | HYSITLRRYAGMALTNLTFGDVANKATLCSMKGCMRALVAQ | |
| | THE ARMADILLO | | LKSESEDLQQVIASVLRNLSWRADVNSKKTLREVGSVKALME | |
| | REPEAT DOMAIN OF | | CALEVKKESTLKSVLSALWNLSAHCTENKADICAVDGALAFL | |
| | ADENOMATOUS | | VGTLTYRSQTNTLAIIESGGGILRNVSSLIATNEDHRQILRENN | |
| | POLYPOSIS COLI AND | | CLQTLLQHLKSHSLTIVSNACGTLWNLSARNPKDQEALWDM | |
| | THE TYROSINE-RICH | | GAVSMLKNLIHSKHKMIAMGSAAALRNLMA | |
| | DOMAIN OF SAM68 | | | |
| 3zke | STRUCTURE OF LC8 | DYNEIN LIGHT | KAVIKNADMSEEMQQDSVECATQALEKYNIEKDIAAHIKKEF | 211 |
| | IN COMPLEX WITH | CHAIN 1, | DKKYNPTWHCIVGRNFGSYVTHETKHFIYFYLGQVAILLFKSG | |
| | NEK9 PEPTIDE | CYTOPLASMIC | | |
| 4htp | CRYSTAL | DNA ligase 4 | SQTVASHVPFADLCSTLERIQKSKGRAEKIRHFREFLDSWRKF | 212 |
| | STRUCTURE OF THE | | HDALHKNHKDVTDSFYPAMRLILPQLERERMAYGIKETMLA | |
| | DBD DOMAIN OF | | KLYIELLNLPRDGKDALKLLNYGDFAMIAYFVLKPRCLQKGS | |
| | HUMAN DNA LIGASE | | LTIQQVNDLLDSIASNNSAKRKDLIKKSLLQLITQSSALEQKWL | |
| | IV BOUND TO | | IRMIIKDLKLGVSQQTIFSVFHNDAAELHNVTTDLEKVCRQLH | |
| | ARTEMIS PEPTIDE | | DPSVGLSD | |
| 4kmd | CRYSTAL | Sufu | SLFPPGLHAIYGECRRLYPDQPNPLQVTAIVKYWLGGPDPLDY | 213 |
| | STRUCTURE OF | | VSMYRNVGSPSANIPEHWHYISFGLSDLYGDNRVHEFTGTDG | |
| | SUFUD60-GLIIP | | PSGFGFELTFRLKRETGESAPPTWPAELMQGLARYVFQSENTF | |
| | | | CSGDHVSWHSPLDNSESRIQHMLLTEDPQMQPVQTPFGVVTF | |
| | | | LQIVGVCTEELHSAQQWNGQGILELLRTVPIAGGPWLITDMRR | |
| | | | GETIFEIDPHLQERVDKGIETDGSNLSGVSAKCAWDDLELIRTR | |
| | | | QLESVHLKFNQESGALIPLCLRGRLLHGRHFTYKSITGDMAITF | |
| | | | VSTGVEGAFATEEHPYAAHGPWLQILLTEEFVEKMLEDLEDL | |
| | | | KLPKEYSWPEKKLKVSILPDVVFDX | |
| 4n39 | CRYSTAL | UDP-N- | CPTHADSLNNLANIKREQGNIEEAVRLYRKALEVFPEFAAAHS | 214 |
| | STRUCTURE OF | acetylglucosam- | NLASVLQQQGKLQEALMHYKEAIRISPTFADAYSNMGNTLKE | |
| | HUMAN O-GLCNAC | e--peptide N- | MQDVQGALQCYTRAIQINPAFADAHSNLASIHKDSGNIPEAIA | |
| | TRANSFERASE | acetylglucosaminyl | SYRTALKLKPDFPDAYCNLAHCLQIVCDWTDYDERMKKLVSI | |
| | BOUND TO A PEPTIDE | transferase 110 | VADQLEKNRLPSVHPHHSMLYPLSHGFRKAIAERHGNLCLDK | |
| | FROM HCF-1 PRO- | kDa subunit | INVLHKPPYEHPKDLKLSDGRLRVGYVSSDFGNHPTSHLMQSI | |
| | REPEAT 2 (11-26) | | PGMHNPDKFEVFCYALSPDDGTNFRVKVMAEANHFIDLSQIP | |
| | | | CNGKAADRIHQDGIHILVNMNGYTKGARNELFALRPAPIQAM | |
| | | | WLGYPGTSGALFMDYIITDQETSPAEVAEQYSEKLAYMPHTF | |
| | | | FIGDHANMFPHLKKKAVIDFKIYDNRIVLNGIDLKAFLDSLPD | |
| | | | VKIVKMLNMPVIPMNTIAEAVIEMINRGQIQITINGFSISNGLAT | |
| | | | TQINNKAATGEEVPRTIIVTTRSQYGLPEDAIVYCNFNQLYKID | |
| | | | PSTLQMWANILKRVPNSVLWLLRFPAVGEPNIQQYAQNMGLP | |
| | | | QNRIIFSPVAPKEEHVRRGQLADVCLDTPLCNGHTTGMDVLW | |
| | | | AGTPMVTMPGETLASRVAASQLTCLGCLELIAKNRQEYEDIA | |
| | | | VKLGTDLEYLKKVRGKVWKQRISSPLFNTKQYTMELERLYLQ | |
| | | | MWEHYAAGNKPDHMIKX | |
| 4y29 | IDENTIFICATION OF | Peroxisome | SADLRALAKHLYDSYIKSFPLTKAKARAILTGKTTDKSPFVIY | 215 |
| | A NOVEL PPARG | proliferator- | DMNSLMMGEDKIKEVAIRIFQGCQFRSVEAVQEITEYAKSIPG | |
| | LIGAND THAT | activated receptor | FVNLDLNDQVTLLKYGVHEIIYTMLASLMNKDGVLISEGQGF | |
| | REGULATES | gamma | MTREFLKSLRKPFGDFMEPKFEFAVKFNALELDDSDLAIFIAVI | |
| | METABOLISM | | ILSGDRPGLLNVKPIEDIQDNLLQALELQLKLNHPESSQLFAKL | |
| | | | LQKMTDLRQIVTEHVQLLQVIKKTETDMSLHPLLQEIYKDLX | |
| 4yje | CRYSTAL | Adenomatous | HHHMLHLLEQIRAYCETCWEWQEAHEPGMDQDKNPMPAPV | 216 |
| | STRUCTURE OF APC- | polyposis coli | EHQICPAVCVLMKLSFDEEHRHAMNELGGLQAIAELLQVDCE | |
| | ARM IN COMPLEXED | protein | MYGLTNDHYSITLRRYAGMALTNLTFGDVANKATLCSMKGC | |
| | WITH AMER1-A1 | | MRALVAQLKSESEDLQQVIASVLRNLSWRADVNSKKTLREV | |
| | | | GSVKALMECALEVKKESTLKSVLSALWNLSAHCTENKADICA | |
| | | | VDGALAFLVGTLTYRSQTNTLAIIESGGGILRNVSSLIATNEDH | |
| | | | RQILRENNCLQTLLQHLKSHSLTIVSNACGTLWNLSARNPKDQ | |
| | | | EALWDMGAVSMLKNLIHSKHKMIAMGSAAALRNLMANRPA | |
| | | | KYK | |
| 5glf | STRUCTURAL | Transitional | NRLIVDEAINEDNSVVSLSQPKMDELQLFRGDTVLLKGKKRR | 217 |
| | INSIGHTS INTO THE | endoplasmic | EAVCIVLSDDTCSDEKIRMNRVVRNNLRVRLGDVISIQPCPDV | |
| | INTERACTION OF P97 | reticulum ATPase | KYGKRIHVLPIDDTVEGITGNLFEVYLKPYFLEAYRPIRKGDIF | |
| | N-TERMINAL | | LVRGGMRAVEFKVVETDPSPYCIVAPDTVIHCEGEPIKRE | |
| | DOMAIN AND SHP | | | |
| | MOTIF IN DERLIN-1 | | | |
| | RHOMBOID | | | |
| | PSEUDOPROTEASE | | | |
| 5h43 | STRUCTURAL AND | Importin subunit | NQGTVNWSVDDIVKGINSSNVENQLQATQAARKLLSREKQPP | 218 |
| | MECHANISTICAL | alpha-1 | IDNIIRAGLIPKFVSFLGRTDCSPIQFESAWALTNIASGTSEQTK | |
| | STUDIES OF THE | | AVVDGGAIPAFISLLASPHAHISEQAVWALGNIAGDGSVFRDL | |
| | NUCLEAR IMPORT BY | | VIKYGAVDPLLALLAVPDMSSLACGYLRNLTWTLSNLCRNKN | |
| | IMPORTIN-ALPHA | | PAPPIDAVEQILPTLVRLLHHDDPEVLADTCWAISYLTDGPNE | |
| | | | RIGMVVKTGVVPQLVKLLGASELPIVTPALRAIGNIVTGTDEQ | |
| | | | TQVVIDAGALAVFPSLLTNPKTNIQKEATWTMSNITAGRQDQI | |
| | | | QQVVNHGLVPFLVSVLSKADFKTQKEAVWAVTNYTSGGTVE | |
| | | | QIVYLVHCGIIEPLMNLLTAKDTKIILVILDAISNIFQAAEKLGE | |
| | | | TEKLSIMIEECGGLDKIEALQNHENESVYKASLSLIEKYFS | |
| 5ni8 | LIGAND COMPLEX OF | Nuclear receptor | ENLYFQGASLTEIEHLVQSVCKSYRETCQLRLEDLLRQRSNIFS | 219 |
| | RORG LBD | ROR-gamma | REEVTGYQRKSMWEMWERCAHHLTEAIQYVVEFAKRLSGF | |
| | | | MELCQNDQIVLLKAGAMEVVLVRMCRAYNADNRTVFFEGK | |
| | | | YGGMELFRALGCSELISSIFDFSHSLSALHFSEDEIALYTALVLI | |
| | | | NAHRPGLQEKRKVEQLQYNLELAFHHHLCKTHRQSILAKLPP | |
| | | | KGKLRSLCSQHVERLQIFQHLHPIVVQAAFPPLYKELFSGGXX | |
| 5y49 | A MODERATOR XD22 | Bile acid receptor | LTPDQQTLLHFIMDSYNKQRMPQEITNKILKEEFSAEENFLILT | 220 |
| | BINDING TO BILE | | EMATNHVQVLVEFTKKLPGFQTLDHEDQIALLKGSAVEAMFL | |
| | ACID RECEPTOR | | RSAEIFNKKLPSGHSDLLEERIRNSGISDEYITPMFSFYKSIGEL | |
| | | | KMTQEEYALLTAIVILSPDRQYIKDREAVEKLQEPLLDVLQKL | |
| | | | CKIHQPENPQHFACLLGRLTELRTFNHHHAEMLMSWRVNDH | |
| | | | KFTPLLCEIWDVX | |
| 1l2z | CD2BP2-GYF DOMAIN | CD2 ANTIGEN | DVMWEYKWENTGDAELYGPFTSAQMQTWVSEGYFPDGVYC | 221 |
| | IN COMPLEX WITH | (CYTOPLASMIC | RKLDPPGGQFYNSKRIDFDLYT | |
| | PROLINE-RICH CD2 | TAIL)-BINDING | | |
| | TAIL SEGMENT | PROTEIN 2 | | |
| | PEPTIDE | | | |
| 1w70 | SH3 DOMAIN OF | NEUTROPHIL | LIKHMRAEALFDFTGNSKLELNFKAGDVIFLLSRINKDWLEGT | 222 |
| | P40PHOX | CYTOSOL | VRGATGIFPLSFVKILKXX | |
| | COMPLEXED WITH C- | FACTOR 4 | | |
| | TERMINAL | | | |
| | POLYPROLINE | | | |
| | REGION OF P47PHOX | | | |
| 2krb | SOLUTION | Eukaryotic | DSVIVVDNVPQVGPDRLEKLKNVIHKIFSKFGKITNDFYPEED | 223 |
| | STRUCTURE OF | translation | GKTKGYIFLEYASPAHAVDAVKNADGYKLDKQHTFRVN | |
| | EIF3B-RRM BOUND | initiation factor 3 | | |
| | TO EIF3J PEPTIDE | subunit B | | |
| 2ks9 | SOLUTION | Substance-P | DNVLPVDSDLSPNISTNTSEPNQFVQPAWQIVLWAAAYTVIVV | 224 |
| | CONFORMATION OF | receptor | TSVVGNVVVMWIILAHKRMRTVTNYFLVNLAFAEASMAAFN | |
| | SUBSTANCE P IN | | TVVNFTYAVHNEWYYGLFYCKFHNFFPIAAVFASIYSMTAVA | |
| | WATER COMPLEXED | | FDRYMAIIHPLQPRLSATATKVVICVIWVLALLLAFPQGYYST | |
| | WITH NKIR | | TETMPSRVVCMIEWPEHPNKIYEKVYHICVTVLIYFLPLLVIGY | |
| | | | AYTVVGITLWASEIPGDSSDRYHEQVSAKRKVVKMMIVVVCT | |
| | | | FAICWLPFHIFFLLPYINPDLYLKKFIQQVYLAIMWLAMSSTM | |
| | | | YNPIIYCCLNDRFRLGFKHAFRCCPFISAGDYEGLEMKSTRYL | |
| | | | QTQGSVYKVSRLETTISTVVGA | |
| 3gn8 | X-RAY CRYSTAL | Glucocorticoid | APTLISLLEVIEPEVLYSGYDSTLPDTSTRLMSTLNRLGGRQVV | 225 |
| | STRUCTURE OF | receptor 2 | SAVKWAKALPGFRNLHLDDQMTLLQYSWMSLMAFSLGWRS | |
| | ANCGR2 IN COMPLEX | | YKQSNGNMLCFAPDLVINEERMQLPYMYDQCQQMLKISSEF | |
| | WITH | | VRLQVSYDEYLCMKVLLLLSTVPKDGLKSQAVFDEIRMTYIK | |
| | DEXAMETHASONE | | ELGKAIVKREGNSSQNWQRFYQLTKLLDSMHEMVGGLLQFC | |
| | | | FYTFVNKSLSVEFPEMLAEIISNQLPKFKAGSVKPLLFHQKX | |
| 3ll8 | CRYSTAL | Serine/threonine- | TDRVVKAVPFPPSHRLTAKEVFDNDGKPRVDILKAHLMKEGR | 226 |
| | STRUCTURE OF | protein | LEESVALRIITEGASILRQEKNLLDIDAPVTVCGDIHGQFFDLM | |
| | CALCINEURIN IN | phosphatase 2B | KLFEVGGSPANTRYLFLGDYVDRGYFSIECVLYLWALKILYPK | |
| | COMPLEX WITH | catalytic subunit | TLFLLRGNHECRHLTEYFTFKQECKIKYSERVYDACMDAFDC | |
| | AKAP79 PEPTIDE | alpha isoform | LPLAALMNQQFLCVHGGLSPEINTLDDIRKLDRFKEPPAYGPM | |
| | | | CDILWSDPLEDFGNEKTQEHFTHNTVRGCSYFYSYPAVCEFLQ | |
| | | | HNNLLSILRAHEAQDAGYRMYRKSQTTGFPSLITIFSAPNYLD | |
| | | | VYNNKAAVLKYENNVMNIRQFNCSPHPYWLPNFMDVFTWSL | |
| | | | PFVGEKVTEMLVNVLNXX | |
| 4wjp | CRYSTAL | Small ubiquitin- | GEYIKLKVIGQDSSEIHFKVKMTTHLKKLKESYAQRQGVPMN | 227 |
| | STRUCTURE OF | related modifier 1 | SLRFLFEGQRIADNHTPKELGMEEEDVIEVYQEQTGG | |
| | SUMO1 IN COMPLEX | | | |
| | WITH | | | |
| | PHOSPHORYLATED | | | |
| | DAXX | | | |
| 4yk6 | CRYSTAL | Adenomatous | HHHMLHLLEQIRAYCETCWEWQEAHEPGMDQDKNPMPAPV | 228 |
| | STRUCTURE OF APC- | polyposis coli | EHQICPAVCVLMKLSFDEEHRHAMNELGGLQAIAELLQVDCE | |
| | ARM IN COMPLEXED | protein | MYGLTNDHYSITLRRYAGMALTNLTFGDVANKATLCSMKGC | |
| | WITH AMER1-A4 | | MRALVAQLKSESEDLQQVIASVLRNLSWRADVNSKKTLREV | |
| | | | GSVKALMECALEVKKESTLKSVLSALWNLSAHCTENKADICA | |
| | | | VDGALAFLVGTLTYRSQTNTLAIIESGGGILRNVSSLIATNEDH | |
| | | | RQILRENNCLQTLLQHLKSHSLTIVSNACGTLWNLSARNPKDQ | |
| | | | EALWDMGAVSMLKNLIHSKHKMIAMGSAAALRNLMANRPA | |
| | | | KYKDAN | |
| 5mf9 | SOLUTION | RNA-binding | GAMGTKYAVPDTSTYQYDESSGYYYDPTTGLYYDPNSQYYY | 229 |
| | STRUCTURE OF THE | protein 5 | NSLTQQYLYWDGEKETYVPAAES | |
| | RBM5 OCRE DOMAIN | | | |
| | IN COMPLEX WITH | | | |
| | POLYPROLINE SMN | | | |
| | PEPTIDE. | | | |
| 2mwp | SOLUTION | Tumor suppressor | GHMNSFVGLRVVAKWSSNGYFYSGKITRDVGAGKYKLLFDD | 230 |
| | STRUCTURE OF 53BP1 | p53-binding | GYECDVLGKDILLCDPIPLDTEVTALSEDEYFSAGVVKGHRKE | |
| | TANDEM TUDOR | protein 1 | SGELYYSIEKEGQRKWYKRMAVILSLEQGNRLREQYGLG | |
| | DOMAINS IN | | | |
| | COMPLEX WITH A | | | |
| | P53K382ME2 PEPTIDE | | | |
| 2rol | STRUCTURAL BASIS | Epidermal growth | GSMGTAGKWVLCNYDFQARNSSELSVKQRDVLEVLDDSRK | 231 |
| | OF PXXDY MOTIF | factor receptor | WWKVRDPAGQEGYVPYNILTPYPG | |
| | RECOGNITION IN SH3 | kinase substrate 8- | | |
| | BINDING | like protein 1 | | |
| 2seb | X-RAY CRYSTAL | HLA CLASS II | IKEEHVIIQAEFYLNPDQSGEFMFDFDGDEIFHVDMAKKETVW | 232 |
| | STRUCTURE OF HLA- | HISTOCOMPATI | RLEEFGRFASFEAQGALANIAVDKANLEIMTKRSNYTPITNVP | |
| | DR4 COMPLEXED | BILITY | PEVTVLTNSPVELREPNVLICFIDKFTPPVVNVTWLRNGKPVT | |
| | WITH A PEPTIDE | ANTIGEN | TGVSETVFLPREDHLFRKFHYLPFLPSTEDVYDCRVEHWGLD | |
| | FROM HUMAN | | EPLLKHWEF | |
| | COLLAGEN II | | | |
| 3plz | HUMAN LRHI LBD | FTZ-F1 related | SIPHLILELLKCEPDEPQVQAKIMAYLQQEKLSTFGLMCKMAD | 233 |
| | BOUND TO GR470 | protein | QTLFSIVEWARSSIFFRELKVDDQMKLLQNCWSELLILDHIYR | |
| | | | QVVHGKEGSIFLVTGQQVDYSIIASQAGATLNNLMSHAQELV | |
| | | | AKLRSLQFDQREFVCLKFLVLFSLDVKNLENFQLVEGVQEQV | |
| | | | NAALLDYTMCNYPQQTEKFGQLLLRLPEIRAISMQAEEYLYY | |
| | | | KHLNGDVPYNNLLIEMLHAX | |
| 4d07 | DYNLL2 DYNEIN | DYNEIN LIGHT | GSHMSDRKAVIKNADMSEDMQQDAVDCATQAMEKYNIEKDI | 234 |
| | LIGHT CHAIN BINDS | CHAIN 2, | AAYIKKEFDKKYNPTWHCIVGRNFGSYVTHETKHFIYFYLGQ | |
| | TO AN EXTENDED, | CYTOPLASMIC | VAILLFKSGXX | |
| | UNSTRUCTURED | | | |
| | LINEAR MOTIF OF | | | |
| | MYOSIN 5A TAIL | | | |
| 4dk7 | CRYSTAL | Oxysterols receptor | HQLTAAQELIQQLVAAQLQCNKRSFSKVTPWPLSRDARQQRF | 235 |
| | STRUCTURE OF LXR | LXR-beta | AHFTELAIISVQEIVDFAKQVPGFLQLGREDQIALLKASTIEILL | |
| | LIGAND BINDING | | ETARRYNHETECITFLKDFTYSKDDFHRAGLQVEFINPIFEFSR | |
| | DOMAIN IN | | ARRLGLDDAEYALLIAINIFSADRPNVQEPGRVEALQQPYVEA | |
| | COMPLEX WITH FULL | | LLSYTRIKRPQDQLRFPRLKLVSLRTLSSVHSEQVFALRLQDK | |
| | AGONIST 1 | | KLPPLLSEIWDVXXXXXX | |
| 4naw | CRYSTAL | Ubiquitin-like | KKIDILLKAVGDTPIMKTKKWAVERTRTIQGLIDFIKKFLKLV | 236 |
| | STRUCTURE OF | protein ATG12 | ASEQLFIYVNQSFAPSPDQEVGTLYECFGSDGKLVLHYCKSQA | |
| | HUMAN | | WG | |
| | ATG12~ATG5-ATG16N | | | |
| | IN COMPLEX WITH A | | | |
| | FRAGMENT OF ATG3 | | | |
| 4p6w | CRYSTAL | Glucocorticoid | PQLTPTLVSLLEVIEPEVLYAGYDSSVPDSTWRIMTTLNMLGG | 237 |
| | STRUCTURE OF | receptor | RQVIAAVKWAKAIPGFRNLHLDDQMTLLQYSWMALMAFAL | |
| | MOMETASONE | | GWRSYRQSSANLLYFAPDLIINEQRMTLPCMYDQCKHMLYVS | |
| | FUROATE-BOUND | | SELHRLQVSYEEYLCMKVLLLLSTIPKDGLKSQELFDEIRMTYI | |
| | GLUCOCORTICOID | | KELGAAIVAREGNSSQNWQRFYQLTKLLDSMHEVVENLLNY | |
| | RECEPTOR LIGAND | | CFQTFLDKTMSIEFPEMLAEIITNQIPKYSNGNIKKLLFHQKX | |
| | BINDING DOMAIN | | | |
| 4qjr | CRYSTAL | Steroidogenic | SGGPNVPELILQLLQLEPDEDQVRARILGSLQEPTKSRPDQPAA | 238 |
| | STRUCTURE OF | factor 1 | FGLLCRMADQTFISIVDWARRCMVFKELEVADQMTLLQNCW | |
| | HUMAN NUCLEAR | | SELLVFDHIYRQVQHGKEGSILLVTGQEVELTTVATQAGSLLH | |
| | RECEPTOR SF-1 | | SLVLRAQELVLQLLALQLDRQEFVCLKFIILFSLDLKFLNNHIL | |
| | (NR5A1) BOUND TO | | VKDAQEKANAALLDYTLCHYPHSGDKFQQLLLCLVEVRALS | |
| | ITS HORMONE PIP3 | | MQAKEYLYHKHLGNEMPRNNLLIEMLQAKQTX | |
| | AT 2.4 A RESOLUTION | | | |
| 4z0r | CRYSTAL | Zinc finger CW- | ENMYVNKVWVQCENENCLKWRLLSSEDSAKVDHDEPWYCF | 239 |
| | STRUCTURE OF THE | type PWWP | MNTDSRYNNCSISEEDRXXXXXXXX | |
| | CW DOMAIN OF | domain protein 2 | | |
| | ZCWPW2 MUTANT | | | |
| | F78R IN COMPLEX | | | |
| | WITH HISTONE H3 | | | |
| | PEPTIDE | | | |
| 5buo | A RECEPTOR | Amyloid beta A4 | ENEHAHFQKAKERLEAKHRERMSQVMREWEEAERQAKNLP | 240 |
| | MOLECULE | protein | KADKKAVIQHFQEKVESLEQEAANERQQLVETHMARVEAML | |
| | | | NDRRRLALENYITALQAVPPRPRHVFNMLKKYVRAEQKDRQ | |
| | | | HTLKHFEHVRMVDPKKAAQIRSQVMTHLRVIYERMNQSLSLL | |
| | | | YNVPAVAEEIQDEVDELLQKEXXXKXXXXX | |
| 1aqd | HLA-DR1 (DRA, DRB1 | HLA-DRI CLASS | RPRFLWQLKFECHFFNGTERVRLLERCIYNQEESVRFDSDVGE | 241 |
| | 0101) HUMAN CLASS | II | YRAVTELGRPDAEYWNSQKDLLEQRRAAVDTYCRHNYGVG | |
| | II | HISTOCOMPATI | ESFTVQRRVEPKVTVYPSKTQPLQHHNLLVCSVSGFYPGSIEV | |
| | HISTOCOMPATIBILITY | BILITY PROTEIN | RWFRNGQEEKAGVVSTGLIQNGDWTFQTLVMLETVPRSGEV | |
| | PROTEIN | | YTCQVEHPSVTSPLTVEWRA | |
| | (EXTRACELLULAR | | | |
| | DOMAIN) | | | |
| | COMPLEXED WITH | | | |
| | ENDOGENOUS | | | |
| | PEPTIDE | | | |
| 1cqg | HIGH RESOLUTION | THIOREDOXIN | MVKQIESKTAFQEALDAAGDKLVVVDFSATWCGPAKMIKPFF | 242 |
| | SOLUTION NMR | | HSLSEKYSNVIFLEVDVDDAQDVASEAEVKATPTFQFFKKGQ | |
| | STRUCTURE OF | | KVGEFSGANKEKLEATINELV | |
| | MIXED DISULFIDE | | | |
| | INTERMEDIATE | | | |
| | BETWEEN HUMAN | | | |
| | THIOREDOXIN (C35A, | | | |
| | C62A, C69A, C73A) | | | |
| | MUTANT AND A 13 | | | |
| | RESIDUE PEPTIDE | | | |
| | COMPRISING ITS | | | |
| | TARGET SITE IN | | | |
| | HUMAN REF-1 | | | |
| | (RESIDUES 59-71 OF | | | |
| | THE P50 SUBUNIT OF | | | |
| | NFKB), NMR, 31 | | | |
| | STRUCTURES | | | |
| 1o0p | SOLUTION | Splicing factor | GHPTEVLCLMNMVLPEELLDDEEYEEIVEDVRDECSKYGLVK | 243 |
| | STRUCTURE OF THE | U2AF 65 kDa | SIEIPRPVDGVEVPGCGKIFVEFTSVFDCQKAMQGLTGRKFAN | |
| | THIRD RNA | subunit | RVVVTKYCDPDSYHRRDFW | |
| | RECOGNITION MOTIF | | | |
| | (RRM) OF U2AF65 IN | | | |
| | COMPLEX WITH AN | | | |
| | N-TERMINAL SF1 | | | |
| | PEPTIDE | | | |
| 3nmx | CRYSTAL | APC variant | HHHMLHLLEQIRAYCETCWEWQEAHNPMPAPVEHQICPAVC | 244 |
| | STRUCTURE OF APC | protein | VLMKLSFDEEHRHAMNELGGLQAIAELLQVDCEMYGLTNDH | |
| | COMPLEXED WITH | | YSITLRRYAGMALTNLTFGDVANKATLCSMKGCMRALVAQL | |
| | ASEF | | KSESEDLQQVIASVLRNLSWRADVNSKKTLREVGSVKALMEC | |
| | | | ALEVKKESTLKSVLSALWNLSAHCTENKADICAVDGALAFLV | |
| | | | GTLTYRSQTNTLAIIESGGGILRNVSSLIATNEDHRQILRENNCL | |
| | | | QTLLQHLKSHSLTIVSNACGTLWNLSARNPKDQEALWDMGA | |
| | | | VSMLKNLIHSKHKMIAMGSAAALRNLMANRPAK | |
| 3wxa | X-RAY CRYSTAL | Programmed cell | QSFLWNVFQRVDKDRSGVISDTELQQALSNGTWTPFNPVTVR | 245 |
| | STRUCTURAL | death protein 6 | SIISMFDRENKAGVNFSEFTGVWKYITDWQNVFRTYDRDNSG | |
| | ANALYSIS OF THE | | MIDKNELKQALSGFGYRLSDQFHDILIRKFDRQGRGQIAFDDFI | |
| | COMPLEX BETWEEN | | QGCIVLQRLTDIFRRYDTDQDGWIQVSYEQYLSMVFSIVXXX | |
| | ALG-2 AND SEC31A | | X | |
| | PEPTIDE | | | |
| 4m7c | CRYSTAL | Telomeric repeat- | ARLEEAVNRWVLKFYFHEALRAFRGSRYGDFRQIRDIMQALL | 246 |
| | STRUCTURE OF THE | binding factor 2 | VRPLGKEHTVSRLLRVMQCLSRIEEGENLDCSFDMEELTPLES | |
| | TRF2-BINDING MOTIF | | AINVLEMIKTEFTLTEAVVESSRKLVKEAAVIICIKNKEFEKAS | |
| | OF SLX4 IN COMPLEX | | KILKKHMPTTQKLRNDLLNIIREKNLAHPVIQNFSYETFQQKM | |
| | WITH THE TRFH | | LRFLESHLDDAEPYLLTMAKKAL | |
| | DOMAIN OF TRF2 | | | |
| 5v3r | CHMP4C IN COMPLEX | Programmed cell | ATFISVQLKKTSEVDLAKPLVKFIQQTYPSGGEEQAQYCRAAE | 247 |
| | WITH ALIX BRO1 | death 6-interacting | ELSKLRRAAVGRPLDKHEGALETLLRYYDQICSIEPKFPFSENQ | |
| | | protein | ICLTFTWKDAFDKGSLFGGSVKLALASLGYEKSCVLFNCAAL | |
| | | | ASQIAAEQNLDNDEGLKIAAKHYQFASGAFLHIKETVLSALSR | |
| | | | EPTVDISPDTVGTLSLIMLAQAQEVFFLKATRDKMKDAIIAKL | |
| | | | ANQAADYFGDAFKQCQYKDTLPKEVFPVLAAKHCIMQANAE | |
| | | | YHQSILAKQQKKFGEEIARLQHAAELIKTVASRYDEYVNVKD | |
| | | | FSDKINRALAAAKKDNDFIYHDRVPDLKDLDPIGKATLVKSTP | |
| | | | VNVPISQKFTDLFEKM | |
| 5xpt | CRYSTAL | Mitotic spindle | RQDLNFGQVVADVLCEFLEVAVHLILYVREVYPVGIFQKRKK | 248 |
| | STRUCTURE OF | assembly | YNVPVQMSCHPELNQYIQDTLHCVKPLLEKNDVEKVVVVILD | |
| | MAD2L2/REV7 IN | checkpoint protein | KEHRPVEKFVFEITQPPLLSISSDSLLSHVEQLLAAFILKISVCD | |
| | COMPLEX WITH A | MAD2B | AVLDHNPPGCTFTVLVHTREAIQVIKDFPWILADEQDVHMHD | |
| | CAMP FRAGMENT IN | | PRLIPLKTMTSDILKMQLYVEERAHK | |
| | A TETRAGONAL | | | |
| | CRYSTAL | | | |
| 6bnh | SOLUTION NMR | Bromodomain- | SEEEDKCKPMSYEEKRQLSLDINKLPGEKLGRVVHIIQSREPSL | 249 |
| | STRUCTURES OF | containing protein | KNSNPDEIEIDFETLKPSTLRELERYVTSCLRKKRKPQA | |
| | BRD4 ET DOMAIN | 4 | | |
| | WITH JMJD6 PEPTIDE | | | |
| 1mv0 | NMR STRUCTURE OF | Myc box- | GRLDLPPGFMFKVQAQHDYTATDTDELQLKAGDVVLVIPFQN | 250 |
| | THE TUMOR | dependent- | PEEQDEGWLMGVKESDWNQHKKLEKCRGVFPENFTERVP | |
| | SUPPRESSOR BINI: | interacting protein | | |
| | ALTERNATIVE | 1 | | |
| | SPLICING IN | | | |
| | MELANOMA AND | | | |
| | INTERACTION WITH | | | |
| | C-MYC | | | |
| 2loz | THE NOVEL BINDING | Tensin-like C1 | MSTAADLLRQGAACSVLYLTSVETESLTGPQAVARASSAALS | 251 |
| | MODE OF DLC1 AND | domain-containing | CSPRPTPAVVHFKVSAQGITLTDNQRKLFFRRHYPVNSITFSST | |
| | TENSIN2 PTB | phosphatase | DPQDRRWTNPDGTTSKIFGFVAKKPGSPWENVCHLFAELDPD | |
| | DOMAIN | | QPAGAIVTFITKVLLGQRK | |
| 3kv4 | STRUCTURE OF PHF8 | PHD finger protein | SVPVYCLCRLPYDVTRFMIECDMCQDWFHGSCVGVEEEKAA | 252 |
| | IN COMPLEX WITH | 8 | DIDLYHCPNCEVLHGPSIMKKKPVKTGSPTFVRELRSRTFDSS | |
| | HISTONE H3 | | DEVILKPTGNQLTVEFLEENSFSVPILVLKKDGLGMTLPSPSFT | |
| | | | VRDVEHYVGSDKEIDVIDVTRQADCKMKLGDFVKYYYSGKR | |
| | | | EKVLNVISLEFSDTRLSNLVETPKIVRKLSWVENLWPEECVFE | |
| | | | RPNVQKYCLMSVRDSYTDFHIDFGGTSVWYHVLKGEKIFYLI | |
| | | | RPTNANLTLFECWSSSSNQNEMFFGDQVDKCYKCSVKQGQT | |
| | | | LFIPTGWIHAVLTPVDCLAFGGNFLHSLNIEMQLKAYEIEKRLS | |
| | | | TADLFRFPNFETICWYVGKHILDIFRGLRENRRHPASYLVHGG | |
| | | | KALNLAFRAWTRKEALPDHEDEIPETVRTVQLIKDLAREIRLV | |
| | | | EDIFQQNXXXXX | |
| 5dbr | CA2+ CAM WITH | Calmodulin | LTEEQIAEFKEAFSLFDKDGDGTITTKELGTVMRSLGQNPTEA | 253 |
| | HUMAN CARDIAC | | ELQDMINEVDADGNGTIDFPEFLTMMARKMKDTDSEEEIREA | |
| | NA+ CHANNEL | | FRVFDKDGNGYISAAELRHVMTNLGEKLTDEEVDEMIREADI | |
| | (NAV1.5) | | DGDGQVNYEEFVQMMTAKXXXX | |
| | INACTIVATION GATE | | | |
| 5x8q | CRYSTAL | Nuclear receptor | SLTEIEHLVQSVCKSYRETCQLRLEDLLRQRSNIFSREEVTGYQ | 254 |
| | STRUCTURE OF THE | ROR-gamma | RKSMWEMWERCAHHLTEAIQYVVEFAKRLSGFMELCQNDQI | |
| | MUTANT HUMAN | | VLLKAGAMEVVLVRMCRAYNADNRTVFFEGKYGGMELFRA | |
| | ROR GAMMA LIGAND | | LGCSELISSIFDFSHSLSALHFSEDEIALYTALVLINAHRPGLQE | |
| | BINDING DOMAIN | | KRKVEQLQYNLELAFHHHLCKTHRQSILAKLPPAGKLASLCS | |
| | WITH ROCKOGENIN. | | QHVERLQIFQHLHPIVX | |
| 6b9h | COMPLEX OF HOOK | Protein Hook | AELCESLLTWIQTFNVDAPCQTVEDLTNGVVMAQVLQKIDPA | 255 |
| | DOMAIN WITH A | homolog 3 | YFDENWLNRIKTEVGDNWRLKISNLKKILKGILDYNHEILGQQ | |
| | DYNEIN LIGHT | | INDFTLPDVNLIGEHSDAAELGRMLQLILGCAVNCEQKQEYIQ | |
| | INTERMEDIATE | | AIMMMEESVQHVVMTAIQELMSK | |
| | CHAIN PEPTIDE | | | |
| 1klg | CRYSTAL | HLA CLASS II | GDTRPRFLWQLKFECHFFNGTERVRLLERCIYNQEESVRFDSD | 256 |
| | STRUCTURE OF HLA- | HISTOCOMPATI | VGEYRAVTELGRPDAEYWNSQKDLLEQRRAAVDTYCRHNY | |
| | DR1/TPI(23-37, THR28- | BILITY | GVGESFTVQRRVEPKVTVYPSKTQHHNLLVCSVSGFYPGSIEV | |
| | ->ILE MUTANT) | ANTIGEN, DR-1 | RWFRNGQEEKAGVVSTGLIQNGDWTFQTLVMLETVPRSGEV | |
| | COMPLEXED WITH | BETA CHAIN | YTCQVEHPSVTSPLTVEWRA | |
| | STAPHYLOCOCCAL | | | |
| | ENTEROTOXIN C3 | | | |
| | VARIANT 3B2 (SEC3- | | | |
| | 3B2) | | | |
| 2lbm | SOLUTION | Transcriptional | GAMADKRGDGLHGIVSCTACGQQVNHFQKDSIYRHPSLQVLI | 257 |
| | STRUCTURE OF THE | regulator ATRX | CKNCFKYYMSDDISRDSDGMDEQCRWCAEGGNLICCDFCHN | |
| | ADD DOMAIN OF | | AFCKKCILRNLGRKELSTIMDENNQWYCYICHPEPLLDLVTAC | |
| | ATRX COMPLEXED | | NSVFENLEQLLQQNKKXXX | |
| | WITH HISTONE TAIL | | | |
| | H3 1-15 K9ME3 | | | |
| 2mg5 | SOLUTION | Calmodulin | ADQLTEEQIAEFKEAFSLFDKDGDGTITTKELGTVMRSLGQNP | 258 |
| | STRUCTURE OF | | TEAELQDMINEVDADGNGTIDFPEFLTMMARKMKDTDSEEEI | |
| | CALMODULIN | | REAFRVFDKDGNGYISAAELRHVMTNLGEKLTDEEVDEMIRE | |
| | BOUND TO THE | | ADIDGDGQVNYEEFVQMMTAK | |
| | TARGET PEPTIDE OF | | | |
| | ENDOTHELIAL | | | |
| | NITROGEN OXIDE | | | |
| | SYNTHASE | | | |
| | PHOSPHORYLATED | | | |
| | AT THR495 | | | |
| 2mwy | MDMX-P53 | Protein Mdm4 | QINQVRPKLPLLKILHAAGAQGEMFTVKEVMHYLGQYIMVK | 259 |
| | | | QLYDQQEQHMVYCGGDLLGELLGRQSFSVKDPSPLYDMLRK | |
| | | | NLVTLAT | |
| 2r28 | THE COMPLEX | Calmodulin | TEEQIAEFKEAFSLFDKDGDGTITTKELGTVMRSLGQNPTEAE | 260 |
| | STRUCTURE OF | | LQDMINEVDADGNGTIDFPEFLTMMARKMKDTDSEEEIREAF | |
| | CALMODULIN | | RVFDKDGNGYISAAELRHVMTNLGEKLTDEEVDEMIREADID | |
| | BOUND TO A | | GDGQVNYEEFVQMMTAXXXX | |
| | CALCINEURIN | | | |
| | PEPTIDE | | | |
| 2x04 | CRYSTAL | POLYADENYLATE- | GPLGSLTASMLASAPPQEQKQMLGERLFPLIQAMHPTLAGKIT | 261 |
| | STRUCTURE OF THE | BINDING | GMLLEIDNSELLHMLESPESLRSKVDEAVAVLQAHQ | |
| | PABC-TNRC6C | PROTEIN 1 | | |
| | COMPLEX | | | |
| 3cfs | STRUCTURAL BASIS | Histone-binding | FEDTVEERVINEEYKIWKKNTPFLYDLVMTHALQWPSLTVQW | 262 |
| | OF THE | protein RBBP7 | LPEVTKPEGKDYALHWLVLGTHTSDEQNHLVVARVHIPNDD | |
| | INTERACTION OF | | TGKIECEIKINHEGEVNRARYMPQNPHIIATKTPSSDVLVFDYT | |
| | RBAP46/RBAP48 WITH | | KHPAKPDPSGECNPDLRLRGHQKEGYGLSWNSNLSGHLLSAS | |
| | HISTONE H4 | | DDHTVCLWDINAGPKEGKIVDAKAIFTGHSAVVEDVAWHLL | |
| | | | HESLFGSVADDQKLMIWDTRSNTTSKPSHLVDAHTAEVNCLS | |
| | | | FNPYSEFILATGSADKTVALWDLRNLKLKLHTFESHKDEIFQV | |
| | | | HWSPHNETILASSGTDRRLNVWDLSKIGEEQSAEDAEDGPPEL | |
| | | | LFIHGGHTAKISDFSWNPNEPWVICSVSEDNIMQIWQMAENIY | |
| | | | NDX | |
| 3l6f | STRUCTURE OF MHC | HLA class II | EEHVIIQAEFYLNPDQSGEFMFDFDGDEIFHVDMAKKETVWR | 263 |
| | CLASS II MOLECULE | histocompatibility | LEEFGRFASFEAQGALANIAVDKANLEIMTKRSNYTPITNVPP | |
| | HLA-DR1 | antigen, DR alpha | EVTVLTNSPVELREPNVLICFIDKFTPPVVNVTWLRNGKPVTT | |
| | COMPLEXED WITH | chain | GVSETVFLPREDHLFRKFHYLPFLPSTEDVYDCRVEHWGLDEP | |
| | PHOSPHOPEPTIDE | | LLKQWEF | |
| | MART-1 | | | |
| 4rav | CRYSTAL | Single-chain Fv, | QSALTQPASVSGSPGQSITISCTGTSSDIGAYNYVSWYQQYPG | 264 |
| | STRUCTURE OF | VL | KAPKLLIYDVSNRPSGISNRFSGSKSGDTASLTISGLQAEDEAD | |
| | SCFVC4 IN COMPLEX | | YYCSSFANSGPLFGGGTKVTVLG | |
| | WITH THE FIRST 17 | | | |
| | AA OF HUNTINGTIN | | | |
| 6d94 | CRYSTAL | Peroxisome | ESADLRALAKHLYDSYIKSFPLTKAKARAILTGKTTDKSPFVIY | 265 |
| | STRUCTURE OF PPAR | proliferator- | DMNSLMMGEDKIKFKHITPLQEQSKEVAIRIFQGCQFRSVEAV | |
| | GAMMA IN COMPLEX | activated receptor | QEITEYAKSIPGFVNLDLNDQVTLLKYGVHEIIYTMLASLMNK | |
| | WITH MEDIATOR OF | gamma | DGVLISEGQGFMTREFLKSLRKPFGDFMEPKFEFAVKFNALEL | |
| | RNA POLYMERASE II | | DDSDLAIFIAVIILSGDRPGLLNVKPIEDIQDNLLQALELQLKLN | |
| | TRANSCRIPTION | | HPESSQLFAKLLQKMTDLRQIVTEHVQLLQVIKKTETDMSLHP | |
| | SUBUNIT 1 | | LLQEIYKDLYX | |
| 2axf | THE | HLA class I | GSHSMRYFYTAMSRPGRGEPRFIAVGYVDDTQFVRFDSDAAS | 266 |
| | IMMUNOGENICITY | histocompatibility | PRTEPRAPWIEQEGPEYWDRNTQIFKTNTQTYRESLRNLRGYY | |
| | OF A VIRAL | antigen, B*3508 | NQSEAGSHIIQRMYGCDLGPDGRLLRGHDQSAYDGKDYIALN | |
| | CYTOTOXIC T CELL | heavy chain | EDLSSWTAADTAAQITQRKWEAARVAEQRRAYLEGLCVEWL | |
| | EPITOPE IS | | RRYLENGKETLQRADPPKTHVTHHPVSDHEATLRCWALGFYP | |
| | CONTROLLED BY ITS | | AEITLTWQRDGEDQTQDTELVETRPAGDRTFQKWAAVVVPS | |
| | MHC-BOUND | | GEEQRYTCHVQHEGLPKPLTLRWEPXX | |
| | CONFORMATION | | | |
| 2bvp | STRUCTURES OF | HLA CLASS I | GSHSMRYFYTAMSRPGRGEPRFIAVGYVDDTQFVRFDSDAAS | 267 |
| | THREE HIV-1 HLA- | HISTOCOMPATI | PRMAPRAPWIEQEGPEYWDGETRNMKASAQTYRENLRIALR | |
| | B5703-PEPTIDE | BILITY | YYNQSEAGSHIIQVMYGCDVGPDGRLLRGHNQYAYDGKDYI | |
| | COMPLEXES AND | ANTIGEN, B-57 | ALNEDLSSWTAADTAAQITQRKWEAARVAEQLRAYLEGLCV | |
| | IDENTIFICATION OF | ALPHA CHAIN | EWLRRYLENGKETLQRADPPKTHVTHHPISDHEATLRCWALG | |
| | RELATED HLAS | | FYPAEITLTWQRDGEDQTQDTELVETRPAGDRTFQKWAAVV | |
| | POTENTIALLY | | VPSGEEQRYTCHVQHEGLPKPLTLRW | |
| | ASSOCIATED WITH | | | |
| | LONG-TERM NON- | | | |
| | PROGRESSION | | | |
| 2f5b | CRYSTAL | PROTEIN | RITLKESGPPLVKPTQTLTLTCSFSGFSLSDFGVGVGWIRQPPG | 268 |
| | STRUCTURE OF FAB′ | (ANTIBODY 2F5 | KALEWLAIIYSDDDKRYSPSLNTRLTITKDTSKNQVVLVMTRV | |
| | FROM THE HIV-1 | (HEAVY | SPVDTATYFCAHRRGPTTLFGVPIARGPVNAMDVWGQGITVT | |
| | NEUTRALIZING | CHAIN)) | ISSTSTKGPSVFPLAPSSAGGAAALGCLVKDYFPEPVTVSWNS | |
| | ANTIBODY 2F5 IN | | GALTSGVHTFPAVLQSSGLYSLSSVVTVPSSSLGTQTYTCNVN | |
| | COMPLEX WITH ITS | | HKPSNTKVDKRVEPKSC | |
| | GP41 EPITOPE | | | |
| 1aln | MHC CLASS I | HLA class I | GSHSMRYFYTAMSRPGRGEPRFIAVGYVDDTQFVRFDSDAAS | 269 |
| | MOLECULE B*3501 | histocompatibility | PRTEPRPPWIEQEGPEYWDRNTQIFKTNTQTYRESLRNLRGYY | |
| | COMPLEXED WITH | antigen, BW-53 | NQSEAGSHIIQRMYGCDLGPDGRLLRGHDQSAYDGKDYIALN | |
| | PEPTIDE VPLRPMTY | B*5301 alpha | EDLSSWTAADTAAQITQRKWEAARVAEQLRAYLEGLCVEWL | |
| | (SEQ ID NO: 579) | chain | RRYLENGKETLQRADPPKTHVTHHPVSDHEATLRCWALGFYP | |
| | FROM THE NEF | | AEITLTWQRDGEDQTQDTELVETRPAGDRTFQKWAAVVVPS | |
| | PROTEIN (75-82) OF | | GEEQRYTCHVQHEGLPKPLTLRWEP | |
| | HIV1 | | | |
| 1agb | ANTAGONIST HIV-1 | B*0801 | GSHSMRYFDTAMSRPGRGEPRFISVGYVDDTQFVRFDSDAAS | 270 |
| | GAG PEPTIDES | | PREEPRAPWIEQEGPEYWDRNTQIFKTNTQTDRESLRNLRGYY | |
| | INDUCE | | NQSEAGSHTLQSMYGCDVGPDGRLLRGHNQYAYDGKDYIAL | |
| | STRUCTURAL | | NEDLRSWTAADTAAQITQRKWEAARVAEQDRAYLEGTCVE | |
| | CHANGES IN HLA B8- | | WLRRYLENGKDTLERADPPKTHVTHHPISDHEATLRCWALGF | |
| | HIV-1 GAG PEPTIDE | | YPAEITLTWQRDGEDQTQDTELVETRPAGDRTFQKWAAVVV | |
| | (GGRKKYKL (SEQ ID | | PSGEEQRYTCHVQHEGLPKPLTLRWEP | |
| | NO: 580)-3R | | | |
| | MUTATION) | | | |
| 1fgl | CYCLOPHILIN A | CYCLOPHILIN A | MVNPTVFFDIAVDGEPLGRVSFELFADKVPKTAENFRALSTGE | 271 |
| | COMPLEXED WITH A | | KGFGYKGSCFHRIIPGFMCQGGDFTRHNGTGGKSIYGEKFEDE | |
| | FRAGMENT OF HIV-1 | | NFILKHTGPGILSMANAGPNTNGSQFFICTAKTEWLDGKHVVF | |
| | GAG PROTEIN | | GKVKEGMNIVEAMERFGSRNGKTSKKITIADCGQLE | |
| 2r7g | STRUCTURE OF THE | Retinoblastoma- | NTIQQLMMILNSASDQPSENLISYFNNCTVNPKESILKRVKDIG | 272 |
| | RETINOBLASTOMA | associated protein | YIFKEKFAKAVGQGCVEIGSQRYKLGVRLYYRVMESMLKSEE | |
| | PROTEIN POCKET | | ERLSIQNFSKLLNDNIFHMSLLACALEVVMATYSRSTGTDLSF | |
| | DOMAIN IN | | PWILNVLNLKAFDFYKVIESFIKAEGNLTREMIKHLERCEHRI | |
| | COMPLEX WITH | | MESLAWLSDSPLFDLIKQSKDKSTSLSLFYKKVYRLAYLRLNT | |
| | ADENOVIRUS EIA | | LCERLLSEHPELEHIIWTLFQHTLQNEYELMRDRHLDQIMMCS | |
| | CR1 DOMAIN | | MYGICKVKNIDLKFKIIVTAYKDLPHAVQETFKRVLIKEEEYD | |
| | | | SIIVFYNSVFMQRLKTNILQYASTRPPTLSPIPHI | |
| 1gux | RB POCKET BOUND | RETINOBLASTOMA | TSLSLFYKKVYRLAYLRLNTLCERLLSEHPELEHIIWTLFQHTL | 273 |
| | TO E7 LXCXE MOTIF | PROTEIN | QNEYELMRDRHLDQIMMCSMYGICKVKNIDLKFKIIVTAYKD | |
| | | | LPHAVQETFKRVLIKEEEYDSIIVFYNSVFMQRLKTNILQYAST | |
| | | | RPPTLSPIPHI | |
| 4o45 | WDR5 IN COMPLEX | WD repeat- | PVKPNYALKFTLAGHTKAVSSVKFSPNGEWLASSSADKLIKI | 274 |
| | WITH INFLUENZA | containing protein | WGAYDGKFEKTISGHKLGISDVAWSSDSNLLVSASDDKTLKI | |
| | NS1 C-TERMINAL | 5 | WDVSSGKCLKTLKGHSNYVFCCNFNPQSNLIVSGSFDESVRI | |
| | TAIL | | WDVKTGKCLKTLPAHSDPVSAVHFNRDGSLIVSSSYDGLCRI | |
| | | | WDTASGQCLKTLIDDDNPPVSFVKFSPNGKYILAATLDNTLKL | |
| | | | WDYSKGKCLKTYTGHKNEKYCIFANFSVTGGKWIVSGSEDN | |
| | | | LVYIWNLQTKEIVQKLQGHTDVVISTACHPTENIIASAALEND | |
| | | | KTIKLWKSDCXXXXXXXXXXX | |
| 4o42 | TANDEM | Chromodomain- | EFETIERFMDCRIGRKGATGATTTIYAVEADGDPNAGFEKNKE | 275 |
| | CHROMODOMAINS | helicase-DNA- | PGEIQYLIKWKGWSHIHNTWETEETLKQQNVRGMKKLDNYK | |
| | OF HUMAN CHD1 IN | binding protein 1 | KKDQETKRWLKNASPEDVEYYNCQQELTDDLHKQYQIVERII | |
| | COMPLEX WITH | | AHSNQKSAAGYPDYYCKWQGLPYSECSWEDGALISKKFQACI | |
| | INFLUENZA NS1 C- | | DEYFSXXXXXXXXXX | |
| | TERMINAL TAIL | | | |
| | DIMETHYLATED AT | | | |
| | K229 | | | |
| 1pyw | HUMAN CLASS II | HLA class II | EHVIIQAEFYLNPDQSGEFMFDFDGDEIFHVDMAKKETVWRL | 276 |
| | MHC PROTEIN HLA- | histocompatibility | EEFGRFASFEAQGALANIAVDKANLEIMTKRSNYTPITNVPPE | |
| | DRI BOUND TO A | antigen, DR alpha | VTVLTNSPVELREPNVLICFIDKFTPPVVNVTWLRNGKPVTTG | |
| | DESIGNED PEPTIDE | chain | VSETVFLPREDHLFRKFHYLPFLPSTEDVYDCRVEHWGLDEPL | |
| | RELATED TO | | LKHWEFDA | |
| | INFLUENZA VIRUS | | | |
| | HEMAGGLUTININ, | | | |
| | FVKQNA(MAA)AL | | | |
| | (SEQ ID NO: 586), IN | | | |
| | COMPLEX WITH | | | |
| | STAPHYLOCOCCAL | | | |
| | ENTEROTOXIN C3 | | | |
| | VARIANT 3B2 (SEC3- | | | |
| | 3B2) | | | |
| 1aot | NMR STRUCTURE OF | FYN PROTEIN- | SIQAEEWYFGKLGRKDAERQLLSFGNPRGTFLIRESETTKGAY | 277 |
| | THE FYN SH2 | TYROSINE | SLSIRDWDDMKGDHVKHYKIRKLDNGGYYITTRAQFETLQQL | |
| | DOMAIN | KINASE | VQHYSERAAGLSSRLVVPSHK | |
| | COMPLEXED WITH A | | | |
| | PHOSPHOTYROSYL | | | |
| | PEPTIDE, MINIMIZED | | | |
| | AVERAGE | | | |
| | STRUCTURE | | | |
| 4i51 | STRUCTURAL | Serine/threonine- | DEKVFTKELDQWIEQLNECKQLSESQVKSLCEKAKEILTKESN | 278 |
| | MECHANISM OF | protein | VQEVRCPVTVCGDVHGQFHDLMELFRIGGKSPDTNYLFMGD | |
| | TRIMERIC PP2A | phosphatase 2A | YVDRGYYSVETVTLLVALKVRYRERITILRGNHESRQITQVYG | |
| | HOLOENZYME | catalytic subunit | FYDECLRKYGNANVWKYFTDLFDYLPLTALVDGQIFCLHGGL | |
| | INVOLVING PR70: | alpha isoform, | SPSIDTLDHIRALDRLQEVPHEGPMCDLLWSDPDDRGGWGISP | |
| | INSIGHT FOR CDC6 | PP2A-alpha | RGAGYTFGQDISETFNHANGLTLVSRAHQLVMEGYNWCHDR | |
| | DEPHOSPHORYLATION | | NVVTIFSAPNYCYRCGNQAAIMELDDTLKYSFLQFDPAPRRG | |
| | | | XXX | |
| 3w8j | CRYSTAL | Protein disulfide- | LYSSSDDVIELTPSNFNREVIQSDSLWLVEFYAPWCGHAQRLT | 279 |
| | STRUCTURE OF P5 A0 | isomerase A6 | PEWKKAATALKDVVKVGAVDADKHHSLGGQYGVQGFPTIKI | |
| | IN A COMPLEX WITH | | FGSNKNRPEDYQGGRTGEAIVDAALSALRQLVKDRLGXX | |
| | PRX4 C-TERM | | | |
| 5jiu | THE CRYSTAL | Microtubule- | NEQEKELQRRLKRLYPAVDEQETPLPRSWSPKDKFSYIGLSQN | 280 |
| | STRUCTURE OF | associated proteins | NLRVHYKGHGKTPKDAASVRATHPIPAACGIYYFEVKIVSKG | |
| | RANBPM/9 IUS-SPRY | 1A/1B light chain | RDGYMGIGLSAQGVNMNRLPGWKDHSYGYHGDDGHSFCSS | |
| | DOMAIN IN | 3B precurso | GTGQPYGPTFTTGDVIGCCVNLINNTCFYTKNGHSLGIAFTDL | |
| | COMPLEX WITH DDX- | | PPNLYPTVGLQTPGEVVDANFGQHPFVFDIEDYMREWRTKIX | |
| | 4 PEPTIDE | | | |
| 2zjd | CRYSTAL | Microtubule- | MPSEKTFKQRRTFEQRVEDVRLIREQHPTKIPVIIERYKGEKQL | 281 |
| | STRUCTURE OF LC3- | associated proteins | PVLDKTKFLVPDHVNMSELIKIIRRRLQLNANQAFFLLVNGHS | |
| | P62 COMPLEX | 1A/1B light chain | MVSVSTPISEVYESEKDEDGFLYMVYASQETFGMK | |
| | | 3B precursor | | |
| 1L2i | HUMAN ESTROGEN | ESTROGEN | SLALSLTADQMVSALLDAEPPILYSEYDPTRPFSEASMMGLLT | 282 |
| | RECEPTOR ALPHA | RECEPTOR | NLADRELVHMINWAKRVPGFVDLTLHDQVHLLECAWLEILM | |
| | LIGAND-BINDING | | IGLVWRSMEHPGKLLFAPNLLLDRNQGKXVEGMVEIFDMLL | |
| | DOMAIN IN | | ATSSRFRMMNLQGEEFVCLKSIILLNSGVYLEEKDHIHRVLDK | |
| | COMPLEX WITH | | ITDTLIHLMAKAGLTLQQQHQRLAQLLLILSHIRHMSNKGME | |
| | (R,R)-5,11-CIS- | | HLYSMKCKNVVPLYDLLLEMLDAHXX | |
| | DIETHYL-5,6, 11,12- | | | |
| | TETRAHYDROCHRYSENE- | | | |
| | 2,8-DIOL AND A | | | |
| | GLUCOCORTICOID | | | |
| | RECEPTOR | | | |
| | INTERACTING | | | |
| | PROTEIN 1 NR BOX II | | | |
| | PEPTIDE | | | |
| 4dma | CRYSTAL | Estrogen receptor | SLALSLTADQMVSALLDAEPPILYSEYDPTRPFSEASMMGLLT | 283 |
| | STRUCTURE OF ERA | | NLADRELVHMINWAKRVPGFVDLTLHDQVHLLECAWLEILM | |
| | LBD IN COMPLEX | | IGLVWRSMEHPGKLLFAPNLLLDRNQGKCVEGMVEIFDMLLA | |
| | WITH RU100132 | | TSSRFRMMNLQGEEFVCLKSIILLNSGVYTFLEEKDHIHRVLD | |
| | | | KITDTLIHLMAKAGLTLQQQHQRLAQLLLILSHIRHMSNKGM | |
| | | | EHLYSMKAKNVVPLYDLLLEMLDAHRLX | |
| 4Lx2 | CRYSTAL | Unconventional | DFQGMLEYKKEDEQKLVKNLILELKPRGVAVNLIPGLPAYILF | 284 |
| | STRUCTURE OF | myosin-Va | MCVRHADYLNDDQKVRSLLTSTINSIKKVLKKRGDDFETVSF | |
| | MY05A GLOBULAR | | WLSNTCRFLHCLKQYSGEEGFMKHNTSRQNEHCLTNFDLAE | |
| | TAIL DOMAIN IN | | YRQVLSDLAIQIYQQLVRVLENILQPMIVSGMLEHEGTYTLDSI | |
| | COMPLEX WITH | | LRQLNSFHSVMCQHGMDPELIKQVVKQMFYIIGAITLNNLLLR | |
| | MELANOPHILIN | | KDMCSWSKGMQIRYNVSQLEEWLRDKNLMNSGAKETLEPLI | |
| | GTBD | | QAAQLLQVKKKTDDDAEAICSMCNALTTAQIVKVLNLYTPV | |
| | | | NEFEERVSVSFIRTIQMRLRDRKDSPQLLMDAKHIFPVTFPFNP | |
| | | | SSLALETIQIPASLGLGFISRVX | |
| 4gxb | STRUCTURE OF THE | Sorting nexin-17 | VPTEEVSLEVLLSNGQKVLVNVLTSDQTEDVLEAVAAKLDLP | 285 |
| | SNX17 ATYPICAL | | DDLIGYFSLFLVREKEDGAFSFVRKLQEFELPYVSVTSLRSQEY | |
| | FERM DOMAIN | | KIVLRKSYWDSAYDDDVMENRVGLNLLYAQTVSDIERGWIL | |
| | BOUND TO THE NPXY | | VTKEQHRQLKSLQEKVSKKEFLRLAQTLRHYGYLRFDACVA | |
| | MOTIF OF P- | | DFPEKDCPVVVSAGNSELSLQLREGSFRVTRMRCWRVTSSVP | |
| | SELECTIN | | LPSGGRGEVRLELAFEYLMSKDRLQWVTITSPQAIMMSICLQS | |
| | | | MVDELMVKKS | |
| 5xxk | STRUCTURE- | E3 ubiquitin- | QIPASEQETLVRPKPLLLKLLKSVGAQKDTYTMKEVLFYLGQ | 286 |
| | ACTIVITY STUDIES | protein ligase | YIATKRLYDEKQQHIVYCSNDLLGDLFGVPSFSVKEHRKIYTM | |
| | OF MDM2/MDM4- | Mdm2 | IYRNLVVVNQ | |
| | BINDING STAPLED | | | |
| | PEPTIDES | | | |
| | COMPRISING NON- | | | |
| | NATURAL AMINO | | | |
| | ACIDS | | | |
| 1thr | STRUCTURES OF | ALPHA- | IVEGSDAEIGMSPWQVMLFRKSPQELLCGASLISDRWVLTAA | 287 |
| | THROMBIN | THROMBIN | HCLLYPPWDKNFTENDLLVRIGKHSRTRYERNIEKISMLEKIYI | |
| | COMPLEXES WITH A | (LARGE | HPRYNWRENLDRDIALMKLKKPVAFSDYIHPVCLPDRETAAS | |
| | DESIGNED AND A | SUBUNIT) | LLQAGYKGRVTGWGNLKETGQPSVLQVVNLPIVERPVCKDST | |
| | NATURAL EXOSITE | | RIRITDNMFCAGYKPDEGKRGDACEGDSGGPFVMKSPFNNR | |
| | INHIBITOR | | WYQMGIVSWGEGCDRDGKYGFYTHVFRLKKWIQKVIDQFGE | |
| 1bd2 | COMPLEX BETWEEN | HLA-A 0201 | GSHSMRYFFTSVSRPGRGEPRFIAVGYVDDTQFVRFDSDAASQ | 288 |
| | HUMAN T-CELL | | RMEPRAPWIEQEGPEYWDGETRKVKAHSQTHRVDLGTLRGY | |
| | RECEPTOR B7, VIRAL | | YNQSEAGSHTVQRMYGCDVGSDWRFLRGYHQYAYDGKDYI | |
| | PEPTIDE (TAX) AND | | ALKEDLRSWTAADMAAQTTKHKWEAAHVAEQLRAYLEGTC | |
| | MHC CLASS I | | VEWLRRYLENGKETLQRTDAPKTHMTHHAVSDHEATLRCW | |
| | MOLECULE HLA-A | | ALSFYPAEITLTWQRDGEDQTQDTELVETRPAGDGTFQKWAA | |
| | 0201 | | VVVPSGQEQRYTCHVQHEGLPKPLTLRWE | |
| 4e5x | CRYSTAL | HLA class I | GSHSMRYFFTSVSRPGRGEPRFIAVGYVDDTQFVRFDSDAASQ | 289 |
| | STRUCTURE OF A | histocompatibility | RMEPRAPWIEQEGPEYWDGETRKVKAHSQTHRVDLGTLRGY | |
| | COMPLEX BETWEEN | antigen, A-2 alpha | YNQSEAGSHTVQRMYGCDVGSDWRFLRGYHQYAYDGKDYI | |
| | THE HUMAN | chain | ALKEDLRSWTAADMAAQTTKHKWEAAHVAEQLRAYLEGTC | |
| | ADENOVIRUS TYPE 2 | | VEWLRRYLENGKETLQRTDAPKTHMTHHAVSDHEATLRCW | |
| | E3- 19K PROTEIN AND | | ALSFYPAEITLTWQRDGEDQTQDTELVETRPAGDGTFQKWAA | |
| | MHC CLASS I | | VVVPSGQEQRYTCHVQHEGLPKPLTLRWE | |
| | MOLECULE HLA- | | | |
| | A2/TAX | | | |
| 2kqf | SOLUTION | Microtubule- | GGSMRPPIIIHRAGKKYGFTLRAIRVYMGDSDVYTVHHMVW | 290 |
| | STRUCTURE OF | associated | HVEDGGPASEAGLRQGDLITHVNGEPVHGLVHTEVVELILKS | |
| | MAST205-PDZ | serine/threonine- | GNKVAISTTPLEN | |
| | COMPLEXED WITH | protein kinase 2 | | |
| | THE C-TERMINUS OF | | | |
| | A RABIES VIRUS G | | | |
| | PROTEIN | | | |
| 1yvh | CRYSTAL | CBL E3 ubiquitin | PGTVDKKMVEKCWKLMDKVVRLCQNPKLALKNSPPYILDLL | 291 |
| | STRUCTURE OF THE | protein ligase | PDTYQHLRTILSRYEGKMETLGENEYFRVFMENLMKKTKQTI | |
| | C-CBL TKB DOMAIN | | SLFKEGKERMYEENSQPRRNLTKLSLIFSHMLAELKGIFPSGLF | |
| | IN COMPLEX WITH | | QGDTFRITKADAAEFWRKAFGEKTIVPWKSFRQALHEVHPISS | |
| | THE APS PTYR-618 | | GLEAMALKSTIDLTCNDYISVFEFDIFTRLFQPWSSLLRNWNSL | |
| | PHOSPHOPEPTIDE | | AVTHPGYMAFLTYDEVKARLQKFIHKPGSYIFRLSCTRLGQW | |
| | | | AIGYVTADGNILQTIPHNKPLFQALIDGFREGFYLFPDGRNQNP | |
| | | | DLTGX | |
| 2iv9 | B2-APPENDAGE | AP-2 COMPLEX | GGYVAPKAVWLPAVKAKGLEISGTFTHRQGHIYMEMNFTNK | 292 |
| | FROM AP2 IN | SUBUNIT BETA- | ALQHMTDFAIQFNKNSFGVIPSTPLAIHTPLMPNQSIDVSLPLN | |
| | COMPLEX WITH | 2 | TLGPVMKMEPLNNLQVAVKNNIDVFYFSCLIPLNVLFVEDGK | |
| | EPS15 PEPTIDE | | MERQVFLATWKDIPNENELQFQIKECHLNADTVSSKLQNNNV | |
| | | | YTIAKRNVEGQDMLYQSLKLTNGIWILAELRIQPGNPNYTLSL | |
| | | | KCRAPEVSQYIYQVYDSILKN | |
| 6mue | VOLTAGE-GATED | Calmodulin-1 | EEQIAEFKEAFSLFDKDGDGTITTKELGTVMRSLGQNPTEAEL | 293 |
| | SODIUM CHANNEL | | QDMINEVDADGNGTIDFPEFLTMMARKMKDTDSEEEIREAFR | |
| | NAV1.4 IQ DOMAIN IN | | VFDKDGNGYISAAELRHVMTNKLTDEEVDEMIREADIDGDGQ | |
| | COMPLEX WITH | | VNYEEFVQMMTAXXXX | |
| | CA2+/CALMODULIN | | | |
| 4p9v | GRB2 SH2 | Growth factor | EMKPHPWFFGKIPRAKAEEMLSKQRHDGAFLIRESESAPGDFS | 294 |
| | COMPLEXED WITH A | receptor-bound | LSVKFGNDVQHFKVLRDGAGKYFLWVVKFNSLNELVDYHRS | |
| | PTYR-AC6CN-ASN | protein 2 | TSVSRNQQIFLRDIEQX | |
| | TRIPEPTIDE | | | |
| 5ttw | CRYSTAL | Polycomb protein | KYSFKCVNSLKEDHNQPLFGVQFNWHSKEGDPLVFATVGSN | 295 |
| | STRUCTURE OF EED | EED | RVTLYECHSQGEIRLLQSYVDADADENFYTCAWTYDSNTSHP | |
| | IN COMPLEX WITH | | LLAVAGSRGIIRIINPITMQCIKHYVGHGNAINELKFHPRDPNL | |
| | UNC4859 | | LLSVSKDHALRLWNIQTDTLVAIFGGVEGHRDEVLSADYDLL | |
| | | | GEKIMSCGMDHSLKLWRINSKRMMNAIKESYDYNPNKTNRP | |
| | | | FISQKIHFPDFSTRDIHRNYVDCVRWLGDLILSKSCENAIVCWK | |
| | | | PGKMEDDIDKIKPSESNVTILGRFDYSQCDIWYMRFSMDFWQ | |
| | | | KMLALGNQVGKLYVWDLEVEDPHKAKCTTLTHHKCGAAIR | |
| | | | QTSFSRDSSILIAVCDDASIWRWDRLXXXX | |
| 6ckz | HUMAN CASPASE-3 | Caspase-3 subunit | HKIPVEADFLYAYSTAPGYYSWRNSKDGSWFIQSLCAMLKQY | 296 |
| | IN COMPLEX WITH | p12 | ADKLEFMHILTRVNRKVATEFESFSFDATFHAKKQIPCIVSML | |
| | AC-DW3-KE | | TKELYFYHLE | |
| 4q13 | APO ESTROGEN | Estrogen receptor | LALSLTADQMVSALLDAEPPILYSEYDPTRPSEASMMGLLTNL | 297 |
| | RECEPTOR ALPHA | | ADRELVHMINWAKRVPGFVDLTLHDQVHLLECAWLEILMIG | |
| | LIGAND BINDING | | LVWRSMEHPGKLLFAPNLLLDRNQGKCVGMVEIFDMLLATS | |
| | DOMAIN D538G | | SRFRMMNLQGEEFVCLKSIILLNSGVYTDHIHRVLDKITDTLIH | |
| | MUTANT WITH A | | LMAKAGLTLQQQHQRLAQLLLILSHIRHMSNKGMEHLYSMK | |
| | GLUCOCORTICOID | | CKNLYGLLLEMLDAHR | |
| | RECEPTOR- | | | |
| | INTERACTING | | | |
| | PROTEIN 1 NR BOX II | | | |
| | PEPTIDE | | | |
| 5jmo | X-RAY STRUCTURE | Furin | YQEPTDPKFPQQWYLSGVTQRDLNVKAAWAQGYTGHGIVVS | 298 |
| | OF FURIN IN | | ILDDGIEKNHPDLAGNYDPGASFDVNDQDPDPQPRYTQMNDN | |
| | COMPLEX WITH THE | | RHGTRCAGEVAAVANNGVCGVGVAYNARIGGVRMLDGEVT | |
| | INHIBITORY | | DAVEARSLGLNPNHIHIYSASWGPEDDGKTVDGPARLAEEAF | |
| | ANTIBODY NB14 | | FRGVSQGRGGLGSIFVWASGNGGREHDSCNCDGYTNSIYTLSI | |
| | | | SSATQFGNVPWYSEACSSTLATTYSSGNQNEKQIVTTDLRQK | |
| | | | CTESHTGTSASAPLAAGIIALTLEANKNLTWRDMQHLVVQTS | |
| | | | KPAHLNANDWATNGVGRKVSHSYGYGLLDAGAMVALAQN | |
| | | | WTTVAPQRKCIIDILTEPKDIGKRLEVRKTVTACLGEPNHITRL | |
| | | | EHAQARLTLSYNRRGDLAIHLVSPMGTRSTLLAARPHDYSAD | |
| | | | GFNDWAFMTTHSWDEDPSGEWVLEIENTSEANNYGTLTKFTL | |
| | | | VLYGTASGSLXXXX | |
| 4wyu | CRYSTAL | Protein scribble | AAALEGPYPVEEIRLPRAGGPLGLSIVGGSDHSSHPFGVQEPG | 299 |
| | STRUCTURE OF | homolog | VFISKVLPRGLAARSGLRVGDRILAVNGQDVRDATHQEAVSA | |
| | SCRIBBLE PDZ34 | | LLRPCLELSLLVRRDPAPPGLRELCIQKAPGERLGISIRGGARG | |
| | TANDEM IN | | HAGNPRDPTDEGIFISKVSPTGAAGRDGRLRVGLRLLEVNQQS | |
| | COMPLEX WITH ITS | | LLGLTHGEAVQLLRSVGDTLTVLVCDGFEXXX | |
| | TARGET PEPTIDE | | | |
| 4xt9 | RORGAMMA (263-509) | Nuclear receptor | ASLTEIEHLVQSVCKSYRETCQLRLEDLLRQRSNIFSREEVTGY | 300 |
| | COMPLEXED WITH | ROR-gamma | QRKSMWEMWERCAHHLTEAIQYVVEFAKRLSGFMELCQND | |
| | GSK2435341A AND | | QIVLLKAGAMEVVLVRMCRAYNADNRTVFFEGKYGGMELFR | |
| | SRC2 | | ALGCSELISSIFDFSHSLSALHFSEDEIALYTALVLINAHRPGLQ | |
| | | | EKRKVEQLQYNLELAFHHHLCKTHRQSILAKLPPKGKLRSLCS | |
| | | | QHVERLQIFQHLHPIVVQAAFPPLYKELFSX | |
| 5bwn | CRYSTAL | NAD-dependent | GKLSLQDVAELIRARACQRVVVMVGAGISTPSGIPDFRSPGSG | 301 |
| | STRUCTURE OF SIRT3 | protein deacetylase | LYSNLQQYDLPYPEAIFELPFFFHNPKPFFTLAKELYPGNYKPN | |
| | WITH A H3K9 | sirtuin-3, | VTHYFLRLLHDKGLLLRLYTQNIDGLERVSGIPASKLVEAHGT | |
| | PEPTIDE | mitochondrial | FASATCTVCQRPFPGEDIRADVMADRVPRCPVCTGVVKPDIVF | |
| | CONTAINING A | | FGEPLPQRFLLHVVDFPMADLLLILGTSLEVEPFASLTEAVRSS | |
| | MYRISTOYL LYSINE | | VPRLLINRDLVGPLAWHPRSRDVAQLGDVVHGVESLVELLG | |
| | | | WTEEMRDLVQRETGKLX | |
| 5ulq | GRB7-SH2 WITH | Growth factor | QLWFHGRISREESQRLIGQQGLVDGLFLVRESQRNPQGFVLSL | 302 |
| | BICYCLIC PEPTIDE | receptor-bound | CHLQKVKHYLILPSEEEGRLYFSMDDGQTRFTDLLQLVEFHQ | |
| | INHIBITOR | protein 7 | LNRGILPCLLRHCCTX | |
| 4x1q | THE CRYSTAL | Urokinase-type | IIGGEFTTIENQPWFAAIYRRHRGGSVTYVCGGSLISPCWVISA | 303 |
| | STRUCTURE OF | plasminogen | THCFIDYPKKEDYIVYLGRSRLNSNTQGEMKFEVENLILHKDY | |
| | MUPAIN-1 IN | activator | SADTLAYHNDIALLKIRSKEGRCAQPSRTIQTIALPSMYNDPQF | |
| | COMPLEX WITH | | GTSCEITGFGKEQSTDYLYPEQLKMTVVKLISHRECQQPHYYG | |
| | MURINISED HUMAN | | SEVTTKMLCAADPQWKTDSCQGDSGGPLVCSLQGRMTLTGI | |
| | UPA AT PH7.4 | | VSWGRGCALKDKPGVYTRVSHFLPWIRSHTKE | |
| 2l3r | NMR STRUCTURE OF | E3 ubiquitin- | GMWDETELGLYKVNEYVDARDTNMGAWFEAQVVRVTRKA | 304 |
| | UHRF1 TANDEM | protein ligase | PSRDEPCSSTSRPALEEDVIYHVKYDDYPENGVVQMNSRDVR | |
| | TUDOR DOMAINS IN | UHRF1 | ARARTIIKWQDLEVGQVVMLNYNPDNPKERGFWYDAEISRK | |
| | A COMPLEX WITH | | RETRTARELYANVVLGDDSLNDCRIIFVDEVFKIERPGE | |
| | HISTONE H3 PEPTIDE | | | |
| 5f8z | THE CRYSTAL | Plasma kallikrein | IVGGTNSSWGEWPWQVSLQVKLTAQRHLCGGSLIGHQWVLT | 305 |
| | STRUCTURE OF | LIGHT CHAIN | AAHCFDGLPLQDVWRIYSGILNLSDITKDTPFSQIKEIIIHQNYK | |
| | HUMAN PLASMA | | VSEGNHDIALIKLQAPLNYTEFQKPISLPSKGDTSTIYTNCWVT | |
| | KALLIKREIN IN | | GWGFSKEKGEIQNILQKVNIPLVTNEECQKRYQDYKITQRMV | |
| | COMPLEX WITH ITS | | CAGYKEGGKDACKGDSGGPLVCKHNGMWRLVGITSWGEGC | |
| | PEPTIDE INHIBITOR | | ARREQPGVYTKVAEYMDWILEKTQSSD | |
| | PKALIN-1 | | | |
| 3ewt | CRYSTAL | Calmodulin | ADQLTEEQIAEFKEAFSLFDKDGDGTITTKELGTVMRSLGQNP | 306 |
| | STRUCTURE OF | | TEAELQDMINEVDADGNGTIDFPEFLTMMARKMKDTDSEEEI | |
| | CALMODULIN | | REAFRVFDKDGNGYISAAELRHVMTNLGEKLTDEEVDEMIRE | |
| | COMPLEXED WITH A | | ADIDGDGQVNYEEFVQMMXXXX | |
| | PEPTIDE | | | |
| 5vb9 | IL-17A IN COMPLEX | Interleukin-17A | GDKNFPRTVMVNLNIHNSDYYDRSTSPWNLHRNEDPERYPSV | 307 |
| | WITH PEPTIDE | | IWEAKCRHLGCINADGNVDYHMNSVPIQQEILVLRREPPHSPN | |
| | | | SFRLEKILVSVGCTCVTPIVHHV | |
| 1bck | HUMAN | PEPTIDYL- | MVNPTVFFDIAVDGEPLGRVSFELFADKVPKTAENFRALSTGE | 308 |
| | CYCLOPHILIN A | PROLYL CIS- | KGFGYKGSCFHRIIPGFMCQGGDFTRHNGTGGKSIYGEKFEDE | |
| | COMPLEXED WITH 2- | TRANS | NFILKHTGPGILSMANAGPNINGSQFFICTAKTEWLDGKHVVF | |
| | THR CYCLOSPORIN | ISOMERASE A | GKVKEGMNIVEAMERFGSRNGKTSKKITIADCGQLE | |
| likf | A CONFORMATION | IGG1-KAPPA | EVKLVESGGGLVQPGGSLKLSCATSGFTFSDYYMYWVRQNS | 309 |
| | OF CYCLOSPORIN A | R45-45-11 FAB | EKRLEWVAFISNGGGSAFYADIVKGRFTISRDNAKNTLYLQM | |
| | IN AQUEOUS | (HEAVY CHAIN) | SRLKSEDTAMYYCTRHTLYDTLYGNYPVWFADWGQGTLVT | |
| | ENVIRONMENT | | VSAAKTTPPSVYPLAPGSAAQTNSMVTLGCLVKGYFPEPVTV | |
| | REVEALED BY THE | | TWNSGSLSSGVHTFPAVLQSDLYTLSSSVTVPSSSRPSETVTCN | |
| | X-RAY STRUCTURE | | VAHPASSTKVDKKIVPRDC | |
| | OF A CYCLOSPORIN- | | | |
| | FAB COMPLEX | | | |
| 2h9m | WDR5 IN COMPLEX | WD-repeat protein | VKPNYALKFTLAGHTKAVSSVKFSPNGEWLASSSADKLIKIW | 310 |
| | WITH UNMODIFIED | 5 | GAYDGKFEKTISGHKLGISDVAWSSDSNLLVSASDDKTLKIW | |
| | H3K4 PEPTIDE | | DVSSGKCLKTLKGHSNYVFCCNFNPQSNLIVSGSFDESVRIWD | |
| | | | VKTGKCLKTLPAHSDPVSAVHFNRDGSLIVSSSYDGLCRIWDT | |
| | | | ASGQCLKTLIDDDNPPVSFVKFSPNGKYILAATLDNTLKLWD | |
| | | | YSKGKCLKTYTGHKNEKYCIFANFSVTGGKWIVSGSEDNLVY | |
| | | | IWNLQTKEIVQKLQGHTDVVISTACHPTENIIASAALENDKTIK | |
| | | | LWKSDC | |

TABLE 2
Functional Classification of Exemplary Human Protein Scaffolds

| PDB | | |
|---|---|---|
| Entry | | |
| ID | Protein Name (Protein Scaffold) | Functional Classification of Protein |
| 1cp3 | APOPAIN | HYDROLASE/HYDROLASE INHIBITOR |
| 1d5x | HLA CLASS II | IMMUNE SYSTEM/PEPTIDE INHIBITOR |
| | HISTOCOMPATIBILITY | |
| | ANTIGEN | |
| 1fch | PEROXISOMAL TARGETING | SIGNALING PROTEIN |
| | SIGNAL 1 RECEPTOR | |
| 1glg | PROTEIN TYROSINE | HYDROLASE, SIGNALING PROTEIN |
| | PHOSPHATASE 1B | |
| 1hcs | HUMAN SRC | COMPLEX (SIGNAL |
| | | TRANSDUCTION/PEPTIDE) |
| 1nop | tyrosyl-DNA phosphodiesterase 1 | HYDROLASE/DNA |
| 1nvq | Serine/threonine-protein kinase Chk1 | TRANSFERASE |
| 1pic | PHOSPHATIDYLINOSITOL 3- | COMPLEX |
| | KINASE | (PHOSPHOTRANSFERASE/RECEPTOR) |
| 1twq | peptidoglycan recognition protein- | IMMUNE SYSTEM, MEMBRANE PROTEIN |
| | I-alpha | |
| 1tzs | Cathepsin E | HYDROLASE |
| 1vbs | CYCLOPHILIN A | ISOMERASE/ISOMERASE SUBSTRATE |
| 1vbt | CYCLOPHILIN A | ISOMERASE/ISOMERASE SUBSTRATE |
| 1zys | Serine/threonine-protein kinase Chk1 | SIGNALING PROTEIN, TRANSFERASE |
| 2g99 | WD-repeat protein 5 | STRUCTURAL PROTEIN/DNA BINDING |
| | | PROTEIN |
| 2h13 | WD-repeat protein 5 | TRANSCRIPTION |
| 2m0u | Na(+)/H(+) exchange regulatory | PEPTIDE BINDING PROTEIN/PROTEIN |
| | cofactor NHE-RF1 | BINDING |
| 2m0v | Na(+)/H(+) exchange regulatory | PROTEIN BINDING/PROTEIN BINDING |
| | cofactor NHE-RF1 | |
| 2o9k | WD repeat protein 5 | GENE REGULATION |
| 2pxj | JmjC domain-containing histone | OXIDOREDUCTASE |
| | demethylation protein 3A | |
| 2q5a | Peptidyl-prolyl cis-trans isomerase | ISOMERASE/ISOMERASE INHIBITOR |
| | NIMA-interacting 1 | |
| 3fvh | Serine/threonine-protein kinase PLK1 | CELL CYCLE, PEPTIDE BINDING PROTEIN |
| 3h11 | Caspase-8 | APOPTOSIS |
| 3idi | 2F5 Fab light chain | IMMUNE SYSTEM |
| 3n7y | Growth factor receptor-bound | PROTEIN BINDING/PEPTIDE |
| | protein 2 | |
| 3oa6 | Male-specific lethal 3 homolog | DNA BINDING PROTEIN/DNA |
| 3rq7 | Serine/threonine-protein kinase | TRANSFERASE/TRANSFERASE |
| | PLK1 | INHIBITOR |
| 4dfw | Serine/threonine-protein kinase PLK1 | TRANSCRIPTION/ANTAGONIST |
| 4dm9 | Ubiquitin carboxyl-terminal | HYDROLASE, LIGASE/INHIBITOR |
| | hydrolase isozyme L1 | |
| 4gm9 | WD repeat-containing protein 5 | TRANSCRIPTION/TRANSCRIPTION |
| | | INHIBITOR |
| 4iim | Intersectin-1 | ENDOCYTOSIS |
| 4joh | Golgi-associated PDZ and coiled-coil | PEPTIDE BINDING PROTEIN |
| | motif-containing protein | |
| 4jr0 | Procaspase-3 | APOPTOSIS, HYDROLASE/HYDROLASE |
| | | INHIBITOR |
| 1a7c | PLASMINOGEN ACTIVATOR | HYDROLASE INHIBITOR/PEPTIDE |
| | INHIBITOR TYPE 1 | |
| 1awq | CYCLOPHILIN A | COMPLEX (ISOMERASE/PEPTIDE) |
| 1bm2 | PROTEIN (GROWTH FACTOR | HORMONE/GROWTH FACTOR |
| | RECEPTOR BOUND PROTEIN 2) | |
| 1f8h | EPIDERMAL GROWTH FACTOR | ENDOCYTOSIS/EXOCYTOSIS |
| | RECEPTOR SUBSTRATE 15 | |
| 1ir3 | INSULIN RECEPTOR | COMPLEX (TRANSFERASE/SUBSTRATE) |
| 1ywt | 14-3-3 protein sigma | SIGNALING PROTEIN/DE NOVO PROTEIN |
| 2oqs | Disks large homolog 1 | PEPTIDE-BINDING PROTEIN |
| 2pie | E3 ubiquitin-protein ligase RNF8 | LIGASE |
| 3kqi | PHD finger protein 2 | NUCLEAR PROTEIN |
| 3tkz | Tyrosine-protein phosphatase non- | HYDROLASE/PEPTIDE |
| | receptor type 11 | |
| 3v4o | Mucosa-associated lymphoid tissue | HYDROLASE/INHIBITOR |
| | lymphoma translocation protein 1 | |
| 4jdi | Serine/threonine-protein kinase | TRANSFERASE/PEPTIDE |
| | PAK 4 | |
| 4nmp | Golgi-associated PDZ and coiled-coil | PROTEIN TRANSPORT/INHIBITOR |
| | motif-containing protein | |
| 4nnm | Tax1-binding protein 3 | PROTEIN BINDING |
| 4q6h | CFTR-associated ligand | TRANSPORT PROTEIN |
| 1f8a | PEPTIDYL-PROLYL CIS-TRANS | ISOMERASE |
| | ISOMERASE NIMA- | |
| | INTERACTING 1 | |
| 1l7z | CALMODULIN | METAL BINDING PROTEIN/PROTEIN |
| | | BINDING |
| 2gfa | Jumonji domain-containing protein | METAL BINDING PROTEIN |
| | 2A | |
| 2ovr | F-box/WD repeat protein 7 | TRANSCRIPTION/CELL CYCLE |
| 3jpv | Proto-oncogene serine/threonine- | TRANSFERASE/TRANSFERASE |
| | protein kinase Pim-1 | INHIBITOR |
| 4E35 | Golgi-associated PDZ and coiled-coil | PROTEIN TRANSPORT/PROTEIN BINDING |
| | motif-containing protein | |
| 4po2 | Heat shock 70 kDa protein 1A/1B | CHAPERONE |
| 1cwd | P56LCK TYROSINE KINASE | PHOSPHOTRANSFERASE/TRANSFERASE |
| | | INHIBI |
| 2qbw | PDZ-Fibronectin fusion protein | UNKNOWN FUNCTION |
| 3ch8 | fusion protein PDZ-Fibronectin | STRUCTURAL PROTEIN |
| 3hna | Histone-lysine N-methyltransferase, | HYDROLASE/HYDROLASE REGULATOR |
| | H3 lysine-9 specific 5 | |
| 4e3b | Tax1-binding protein 3 | SIGNALING PROTEIN/PROTEIN BINDING |
| 4okw | Androgen receptor | HORMONE RECEPTOR/PEPTIDE |
| 1a9e | HLA CLASS I | COMPLEX (MHC CLASS I/PEPTIDE) |
| | HISTOCOMPATIBILITY | |
| | ANTIGEN, B-35 B*3501 (ALPHA | |
| | CHAIN) | |
| 1cf0 | PROTEIN (PROFILIN) | COMPLEX (ACTIN-BINDING |
| | | PROTEIN/PEPTID |
| 1eeo | PROTEIN TYROSINE | HYDROLASE/HYDROLASE SUBSTRATE |
| | PHOSPHATASE 1B | |
| 1hsa | CLASS I HISTOCOMPATIBILITY | HISTOCOMPATIBILITY ANTIGEN |
| | ANTIGEN (HLA-B*2705) | |
| 3fe7 | Mdm4 protein | CELL CYCLE |
| 3kpq | HLA class I histocompatibility | IMMUNE SYSTEM/PEPTIDE |
| | antigen, B-44 alpha chain | |
| 4jij | Matrix metalloproteinase-9 | HYDROLASE/SUBSTRATE |
| 1aqc | X11 | COMPLEX (PEPTIDE BINDING |
| | | MODULE/PEPTI |
| 2axi | Ubiquitin-protein ligase E3 Mdm2 | LIGASE/LIGASE INHIBITOR |
| 2o02 | 14-3-3 protein zeta/delta | PROTEIN BINDING/TOXIN |
| 3p9g | Tumor susceptibility gene 101 protein | PROTEIN TRANSPORT |
| 3qhr | Cell division protein kinase 2 | TRANSFERASE/PROTEIN BINDING |
| 3sfj | Tax1-binding protein 3 | SIGNALING PROTEIN/INHIBITOR |
| 4j9c | Tyrosine-protein kinase ABL1 | TRANSFERASE/UNKNOWN FUNCTION |
| 4rxz | Protein Mdm4 | INHIBITOR |
| 1ov3 | Neutrophil cytosol factor 1 | OXIDOREDUCTASE ACTIVATOR |
| 2p8l | nmAb 2F5, light chain | VIRAL PROTEIN |
| 3p72 | Platelet glycoprotein Ib alpha chain | BLOOD CLOTTING/INHIBITOR |
| 1y98 | Breast cancer type 1 susceptibility | ANTITUMOR PROTEIN |
| | protein | |
| 2lgk | E3 ubiquitin-protein ligase UHRF1 | LIGASE/DNA BINDING PROTEIN |
| 3eqy | Mdm4 protein | ONCOPROTEIN |
| 2oj2 | Hematopoetic Cell Kinase, SH3 | SIGNALING |
| | domain | PROTEIN, TRANSFERASE/INHIBIT |
| 3d8c | Hypoxia-inducible factor 1 alpha | TRANSCRIPTION REGULATOR, |
| | inhibitor | OXIDOREDUCTA |
| 2qki | Complement C3 | IMMUNE SYSTEM/HYDROLASE |
| | | INHIBITOR |
| 4fmq | Mitogen-activated protein kinase 1 | TRANSFERASE/SIGNALING PROTEIN |
| 2m3m | Disks large homolog 1 | ONCOPROTEIN/CELL ADHESION |
| 5ic3 | Golgi-associated PDZ and coiled-coil | PEPTIDE BINDING PROTEIN/INHIBITOR |
| | motif-containing protein | |
| 2kpl | Membrane-associated guanylate | PROTEIN BINDING/ONCOPROTEIN |
| | kinase, WW and PDZ domain- | |
| | containing protein 1 | |
| 4pz5 | Fibronectin | CELL ADHESION |
| 2l7u | Advanced glycosylation end product- | ALLERGEN |
| | specific receptor | |
| 6hy7 | Neuronal acetylcholine receptor | TOXIN |
| | subunit alpha-9 | |
| 4lcy | HLA class I histocompatibility | MEMBRANE PROTEIN |
| | antigen, B-46 alpha chain | |
| 3hql | Speckle-type POZ protein | PROTEIN BINDING, LIGASE/HYDROLASE |
| 3hqm | Speckle-type POZ protein | PROTEIN BINDING, LIGASE |
| 2ff6 | Gelsolin | STRUCTURAL PROTEIN/CONTRACTILE |
| | | PROTEI |
| 3f2o | SPRY domain-containing SOCS box | APOPTOSIS/HYDROLASE |
| | protein 1 | |
| 2cia | CYTOPLASMIC PROTEIN NCK2 | SH2-DOMAIN |
| 5h7g | B-cell lymphoma 6 protein | TRANSCRIPTION/INHIBITOR |
| 5h5q | Phospholipid hydroperoxide | OXIDOREDUCTASE/OXIDOREDUCTASE |
| | glutathione peroxidase, mitochondrial | INHIBIT |
| 1bt6 | S100A10 | COMPLEX (LIGAND/ANNEXIN) |
| 6d3z | Plasminogen | BLOOD CLOTTING |
| 3iec | Serine/threonine-protein kinase | SIGNALING PROTEIN/TOXIN |
| | MARK2 | |
| 3ua7 | Tyrosine-protein kinase Fyn | TRANSFERASE/VIRAL PROTEIN |
| 4j1v | MOB kinase activator 1A | PEPTIDE BINDING PROTEIN/VIRAL |
| | | PROTEIN |
| 5jzi | HLA class I histocompatibility | PROTEIN BINDING/IMMUNE SYSTEM |
| | antigen, A-2 alpha chain | |
| 4jzo | Anti-HCV E2 Fab HC84-27 light | IMMUNE SYSTEM/VIRAL PROTEIN |
| | chain | |
| 5kzp | HCV1-C1 Antibody Fab Light Chain | VIRAL PROTEIN/IMMUNE SYSTEM |
| 3zhf | AP-1 COMPLEX SUBUNIT | PROTEIN TRANSPORT/VIRAL PROTEIN |
| | GAMMA-LIKE 2 | |
| 1lhc | ALPHA-THROMBIN | COMPLEX (SERINE |
| | | PROTEASE/INHIBITOR) |
| 3d49 | Thrombin heavy chain | BLOOD CLOTTING/HYDROLASE |
| | | INHIBITOR |
| 1ai8 | ALPHA-THROMBIN (LARGE | BLOOD COAGULATION/HYDROLASE |
| | SUBUNIT) | INHIBITOR |
| 1hah | ALPHA-THROMBIN (LARGE | COMPLEX(SERINE |
| | SUBUNIT) | PROTEINASE/INHIBITOR) |
| 1hgt | ALPHA-THROMBIN (LARGE | HYDROLASE(SERINE PROTEASE) |
| | SUBUNIT) | |
| 1hxe | THROMBIN | HYDROLASEE/HYDROLASE INHIBITOR |
| 1zrb | thrombin | BLOOD |
| | | CLOTTING, HYDROLASE/INHIBITOR |
| 1mue | THROMBIN | HYDROLASE INHIBITOR |
| 1bhx | ALPHA THROMBIN | SERINE PROTEASE |
| 1h9o | PHOSPHATIDYLINOSITOL 3- | TRANSFERASE/RECEPTOR |
| | KINASE | |
| 2aij | Sulfatase modifying factor 1 | HYDROLASE ACTIVATOR, PROTEIN |
| | | BINDING |
| 2ce9 | TRANSDUCIN-LIKE ENHANCER | TRANSCRIPTION REGULATION |
| | PROTEIN 1 | |
| 2hi8 | Sulfatase-modifying factor 1 | HYDROLASE ACTIVATOR, PROTEIN |
| | | BINDING |
| 3cbo | Histone-lysine N-methyltransferase | TRANSFERASE/TRANSFERASE |
| | SETD7 | RECEPTOR |
| 3E50 | Insulin-degrading enzyme | HYDROLASE/HORMONE |
| 3k2u | Hepatocyte growth factor activator | HYDROLASE/IMMUNE SYSTEM |
| | long chain | |
| 3o35 | Transcription intermediary factor | TRANSCRIPTION/PROTEIN BINDING |
| | 1-alpha | |
| 3qj6 | Hepatoma-derived growth factor- | PROTEIN BINDING/NUCLEAR PROTEIN |
| | related protein 2 | |
| 3rbq | Protein unc-119 homolog A | PROTEIN TRANSPORT/LIPID BINDING |
| | | PROTE |
| 3rl7 | Disks large homolog 1 | MEMBRANE PROTEIN/SIGNALING |
| | | PROTEIN |
| 3vro | Signal transduction protein CBL-C | PROTEIN BINDING/TRANSFERASE |
| 3zdy | INTEGRIN BETA-3 | CELL ADHESION/IMMUNE |
| | | SYSTEM/PEPTIDE |
| 3zvy | E3 UBIQUITIN-PROTEIN LIGASE | LIGASE/PEPTIDE |
| | UHRF1 | |
| 4n3w | CREB-binding protein | TRANSFERASE/NUCLEAR PROTEIN |
| 4ny3 | Serine/threonine-protein phosphatase | HYDROLASE ACTIVATOR |
| | 2A activator | |
| 4ztd | Proliferating cell nuclear antigen | REPLICATION |
| 5c27 | Tyrosine-protein kinase SYK | TRANSFERASE/INHIBITOR |
| 5ulo | 14-3-3 protein zeta/delta | SIGNALING PROTEIN/PEPTIDE |
| 6bfj | Caspase-3 | APOPTOSIS/INHIBITOR |
| 6dn6 | SPRY domain-containing SOCS box | IMMUNE SYSTEM/INHIBITOR |
| | protein 2 | |
| 6j8f | Tubulinyl-Tyr carboxypeptidase 1 | PEPTIDE BINDING PROTEIN/HYDROLASE |
| 1csy | SYK PROTEIN TYROSINE | COMPLEX |
| | KINASE | (PHOSPHOTRANSFERASE/PEPTIDE) |
| 1tw6 | Baculoviral IAP repeat-containing | INHIBITOR/APOPTOSIS |
| | protein 7 | |
| 2ain | Afadin | CELL ADHESION/TRANSFERASE |
| 2br9 | 14-3-3 PROTEIN EPSILON | CELL REGULATOR PROTEIN |
| 2eax | Peptidoglycan recognition protein- | PEPTIDOGLYCAN-BINDING PROTEIN |
| | I-beta | |
| 2ri7 | Nucleosome-remodeling factor | TRANSCRIPTION/NUCLEAR PROTEIN |
| | subunit BPTF | |
| 3iqu | 14-3-3 protein sigma | PROTEIN BINDING, SIGNALING PROTEIN |
| 3lny | Tyrosine-protein phosphatase non- | SIGNALING PROTEIN/SIGNALING |
| | receptor type 13 | PROTEIN |
| 3u4s | Lysine-specific demethylase 4A | OXIDOREDUCTASE/OXIDOREDUCTASE |
| | | SUBSTRA |
| 4bve | NAD-DEPENDENT PROTEIN | HYDROLASE/LIGASE |
| | DEACETYLASE SIRTUIN-3, | |
| | MITOCHONDRIAL | |
| 4fr3 | 14-3-3 protein sigma | PEPTIDE BINDING PROTEIN/TOXIN |
| 4hix | Humanized 3D6 Fab light chain | PROTEIN FIBRIL/IMMUNE SYSTEM |
| 4o62 | Zinc finger CW-type PWWP domain | METAL/DNA BINDING PROTEIN |
| | protein 2 | |
| 4pxm | Estrogen receptor | HORMONE/PEPTIDE |
| 4rh5 | Tyrosine-protein phosphatase non- | HYDROLASE/PROTEIN BINDING |
| | receptor type 3 | |
| 4wk2 | Integrin beta-1 | CELL ADHESION/IMMUNE SYSTEM |
| 5eg2 | Histone-lysine N-methyltransferase | TRANSFERASE/TRANSCRIPTION FACTOR |
| | SETD7 | |
| 5f2u | Retinal rod rhodopsin-sensitive | LIPID BINDING PROTEIN |
| | cGMP 3′,5′-cyclic phosphodiesterase | |
| | subunit delta | |
| 5ggp | Protein O-linked-mannose beta-1,2- | SUGAR BINDING PROTEIN |
| | N-acetylglucosaminyltransferase 1 | |
| 5lso | Splicing factor 45 | SPLICING/INHIBITOR |
| 5xup | Telomeric repeat-binding factor 1 | DNA BINDING PROTEIN |
| 1ca9 | PROTEIN (TNF RECEPTOR | TNF SIGNALING |
| | ASSOCIATED FACTOR 2) | |
| 1qjb | 14-3-3 PROTEIN ZETA/DELTA | KINASE INHIBITOR/PEPTIDE |
| 1ujk | ADP-ribosylation factor binding | PROTEIN TRANSPORT/HYDROLASE |
| | protein GGA1 | |
| 3bun | E3 ubiquitin-protein ligase CBL | LIGASE/SIGNALING PROTEIN |
| 3hqu | Egl nine homolog 1 | OXIDOREDUCTASE/TRANSCRIPTION |
| 3qlc | Transcriptional regulator ATRX | TRANSCRIPTION/STRUCTURAL PROTEIN |
| 3shb | E3 ubiquitin-protein ligase UHRF1 | LIGASE/NUCLEAR PROTEIN |
| 3sov | Low-density lipoprotein receptor- | PROTEIN BINDING/ANTAGONIST |
| | related protein 6 | |
| 3v43 | Histone acetyltransferase KAT6A | TRANSFERASE/STRUCTURAL PROTEIN |
| 4es0 | WD repeat-containing protein 5 | TRANSCRIPTION/TRANSFERASE |
| 4l1u | RNA polymerase-associated protein | TRANSCRIPTION/PEPTIDE |
| | RTF1 homolog | |
| 4l7x | Death-inducer obliterator 1 | CELL CYCLE, GENE REGULATION |
| 4mvk | Neutrophil gelatinase-associated | PROTEIN BINDING/PROTEIN FIBRIL |
| | lipocalin | |
| 6jwm | SPRY domain-containing SOCS box | PROTEIN BINDING/INHIBITOR |
| | protein 2 | |
| 1gjb | UROKINASE-TYPE | BLOOD CLOTTING, HYDROLASE |
| | PLASMINOGEN ACTIVATOR | |
| 2b1z | Estrogen receptor | HORMONE/GROWTH FACTOR RECEPTOR |
| 2kbs | Harmonin | STRUCTURAL PROTEIN/CELL ADHESION |
| 2rt5 | Msx2-interacting protein | TRANSCRIPTION REGULATOR |
| 2w3o | BIFUNCTIONAL | HYDROLASE, TRANSFERASE/PEPTIDE |
| | POLYNUCLEOTIDE | |
| | PHOSPHATASE/KINASE | |
| 3t03 | Peroxisome proliferator-activated | TRANSCRIPTION/TRANSCRIPTION |
| | receptor gamma | REGULATOR |
| 4bxf | BIFUNCTIONAL LYSINE- | OXIDOREDUCTASE/TRANSLATION |
| | SPECIFIC DEMETHYLASE AND | |
| | HISTIDYL-HYDROXYLASE MINA | |
| 4hrg | Protein S100-A10 | CALCIUM-BINDING PROTEIN |
| 4lxl | Lysine-specific demethylase 4B | OXIDOREDUCTASE/PEPTIDE |
| 6e3g | Nuclear receptor ROR-gamma | NUCLEAR PROTEIN/AGONIST |
| 6uyz | Small ubiquitin-related modifier 1 | NUCLEAR PROTEIN/PROTEIN BINDING |
| 1gwq | OESTROGEN RECEPTOR | NUCLEAR RECEPTOR |
| 1lck | P56 == LCK == TYROSINE KINASE | COMPLEX (KINASE/PEPTIDE) |
| 1oga | T-CELL RECEPTOR BETA CHAIN | IMMUNE SYSTEM/RECEPTOR |
| | C REGION | |
| 1qew | PROTEIN (HLA CLASS I | COMPLEX (MHC PROTEIN/ANTIGEN) |
| | HISTOCOMPATIBILITY | |
| | ANTIGEN, B-35 B* 3501 ALPHA | |
| | CHAIN) | |
| 1xr9 | HLA class I histocompatibility | IMMUNE SYSTEM/LIGASE |
| | antigen, B-15 alpha chain | |
| 2cbl | PROTO-ONCOGENE CBL | COMPLEX (PROTO-ONCOGENE/PEPTIDE) |
| 2d1x | cortactin isoform a | CELL INVASION |
| 2y1n | E3 UBIQUITIN-PROTEIN LIGASE | LIGASE/TRANSFERASE |
| 3al3 | DNA topoisomerase 2-binding | DNA BINDING PROTEIN/PROTEIN |
| | protein 1 | BINDING |
| 3uu7 | Estrogen receptor | HORMONE RECEPTOR |
| 3uzd | 14-3-3 protein gamma | PROTEIN BINDING/HYDROLASE |
| 4ch9 | KELCH-LIKE PROTEIN 3 | SIGNALING PROTEIN/TRANSFERASE |
| 4jyi | Retinoic acid receptor beta | TRANSCRIPTION/AGONIST |
| 4nnx | HLA class I histocompatibility | IMMUNE SYSTEM/ANTIGEN |
| | antigen, A-2 alpha chain | |
| 5E50 | Aprataxin and PNK-like factor | LYASE |
| 6ivx | Nuclear receptor ROR-gamma | NUCLEAR PROTEIN/INHIBITOR |
| 1mw4 | Growth factor receptor-bound | HORMONE/GROWTH |
| | protein 7 | FACTOR/TRANSFERASE |
| 1om9 | ADP-ribosylation factor binding | PROTEIN TRANSPORT, SIGNALING |
| | protein GGA1 | PROTEIN |
| 2o9v | Ponsin | SIGNALING PROTEIN/CELL ADHESION |
| 2q7i | Androgen receptor | HORMONE |
| 2r0l | Hepatocyte growth factor activator | HYDROLASE, IMMUNE SYSTEM |
| 2rr4 | Zinc finger CW-type PWWP domain | METAL-BINDING PROTEIN/NUCLEAR |
| | protein 1 | PROTEIN |
| 3oll | Estrogen receptor beta | HORMONE RECEPTOR/ACTIVATOR |
| 3pfv | E3 ubiquitin-protein ligase CBL-B | LIGASE/PROTEIN BINDING |
| 3qhe | Adenomatous polyposis coli protein | SIGNALING PROTEIN/SPLICING |
| 3zke | DYNEIN LIGHT CHAIN 1, | CONTRACTILE PROTEIN/PEPTIDE |
| | CYTOPLASMIC | |
| 4htp | DNA ligase 4 | LIGASE/HYDROLASE |
| 4kmd | Sufu | PROTEIN BINDING/TRANSCRIPTION |
| 4n39 | UDP-N-acetylglucosamine--peptide | TRANSFERASE/SUBSTRATE |
| | N-acetylglucosaminyltransferase 110 | |
| | kDa subunit | |
| 4y29 | Peroxisome proliferator-activated | DNA BINDING PROTEIN/TRANSCRIPTION |
| | receptor gamma | |
| 4yje | Adenomatous polyposis coli protein | CELL ADHESION/PROTEIN BINDING |
| 5glf | Transitional endoplasmic reticulum | HYDROLASE/TRANSPORT PROTEIN |
| | ATPase | |
| 5h43 | Importin subunit alpha-1 | PROTEIN TRANSPORT/TRANSFERASE |
| 5ni8 | Nuclear receptor ROR-gamma | RAR-RELATED ORPHAN RECEPTOR-G |
| | | (RORG) |
| 5y49 | Bile acid receptor | TRANSCRIPTION/TRANSCRIPTION |
| | | ACTIVATOR |
| 1l2z | CD2 ANTIGEN (CYTOPLASMIC | PEPTIDE BINDING/SIGNALING PROTEIN |
| | TAIL)-BINDING PROTEIN 2 | |
| 1w70 | NEUTROPHIL CYTOSOL FACTOR | SH3 DOMAIN |
| | 4 | |
| 2krb | Eukaryotic translation initiation factor | TRANSLATION |
| | 3 subunit B | |
| 2ks9 | Substance-P receptor | NEUROPEPTIDE |
| | | RECEPTOR/NEUROPEPTIDE |
| 3gn8 | Glucocorticoid receptor 2 | HORMONE/HORMONE ACTIVATOR |
| 3ll8 | Serine/threonine-protein phosphatase | HYDROLASE/CALCIUM BINDING |
| | 2B catalytic subunit alpha isoform | PROTEIN |
| 4wjp | Small ubiquitin-related modifier 1 | PROTEIN BINDING/SIGNALING PROTEIN |
| 4yk6 | Adenomatous polyposis coli protein | PROTEIN BINDING/CELL ADHESION |
| 5mf9 | RNA-binding protein 5 | SPLICING |
| 2mwp | Tumor suppressor p53-binding | TRANSCRIPTION/ANTITUMOR PROTEIN |
| | protein 1 | |
| 2rol | Epidermal growth factor receptor | SPLICING/SIGNALING PROTEIN |
| | kinase substrate 8-like protein 1 | |
| 2seb | HLA CLASS II | COMPLEX (MHC CLASS |
| | HISTOCOMPATIBILITY | II/SUPERANTIGEN) |
| | ANTIGEN | |
| 3plz | FTZ-F1 related protein | TRANSCRIPTION/RECEPTOR/AGONIST |
| 4d07 | DYNEIN LIGHT CHAIN 2, | MOTOR PROTEIN |
| | CYTOPLASMIC | |
| 4dk7 | Oxysterols receptor LXR-beta | TRANSCRIPTION/PEPTIDE/AGONIST |
| 4naw | Ubiquitin-like protein ATG12 | PROTEIN TRANSPORT/LIGASE |
| 4p6w | Glucocorticoid receptor | HORMONE RECEPTOR/HORMONE |
| | | ACTIVATOR |
| 4qjr | Steroidogenic factor 1 | TRANSCRIPTION FACTOR/HORMONE |
| 4z0r | Zinc finger CW-type PWWP domain | PROTEIN BINDING/STRUCTURAL |
| | protein 2 | PROTEIN |
| 5buo | Amyloid beta A4 protein | METAL TRANSPORT |
| 1aqd | HLA-DR1 CLASS II | COMPLEX (MHC PROTEIN/ANTIGEN) |
| | HISTOCOMPATIBILITY PROTEIN | |
| 1cqg | THIOREDOXIN | COMPLEX (ELECTRON |
| | | TRANSPORT/PEPTIDE) |
| 1o0p | Splicing factor U2AF 65 kDa subunit | RNA BINDING PROTEIN |
| 3nmx | APC variant protein | CELL ADHESION/CELL CYCLE |
| 3wxa | Programmed cell death protein 6 | APOPTOSIS/TRANSPORT PROTEIN |
| 4m7c | Telomeric repeat-binding factor 2 | HYDROLASE REGULATOR |
| 5v3r | Programmed cell death 6-interacting | CELL DIVISION, TRANSPORT PROTEIN |
| | protein | |
| 5xpt | Mitotic spindle assembly checkpoint | TRANSCRIPTION/METAL BINDING |
| | protein MAD2B | PROTEIN |
| 6bnh | Bromodomain-containing protein 4 | TRANSCRIPTION/OXIDOREDUCTASE |
| 1mv0 | Myc box-dependent-interacting | ENDOCYTOSIS/EXOCYTOSIS, |
| | protein 1 | TRANSCRIPTION |
| 2loz | Tensin-like C1 domain-containing | HYDROLASE/HYDROLASE ACTIVATOR |
| | phosphatase | |
| 3kv4 | PHD finger protein 8 | H3K4ME3 BINDING PROTEIN, |
| | | TRANSFERASE |
| 5dbr | Calmodulin | METAL BINDING PROTEIN/TRANSPORT |
| | | PROTE |
| 5x8q | Nuclear receptor ROR-gamma | TRANSFERASE/IHIBITOR |
| 6b9h | Protein Hook homolog 3 | PROTEIN TRANSPORT/MOTOR PROTEIN |
| 1klg | HLA CLASS II | IMMUNE SYSTEM/TOXIN |
| | HISTOCOMPATIBILITY | |
| | ANTIGEN, DR-1 BETA CHAIN | |
| 2lbm | Transcriptional regulator ATRX | METAL BINDING PROTEIN/STRUCTURAL |
| | | PROT |
| 2mg5 | Calmodulin | METAL BINDING PROTEIN/TARGET |
| | | PEPTIDE |
| 2mwy | Protein Mdm4 | CELL CYCLE/ANTITUMOR PROTEIN |
| 2r28 | Calmodulin | METAL BINDING PROTEIN/HYDROLASE |
| 2x04 | POLYADENYLATE-BINDING | PEPTIDE/RNA BINDING PROTEIN |
| | PROTEIN 1 | |
| 3cfs | Histone-binding protein RBBP7 | HISTONE/CHAPERONE |
| 3l6f | HLA class II histocompatibility | IMMUNE SYSTEM/PEPTIDE BINDING |
| | antigen, DR alpha chain | PROTEIN |
| 4rav | Single-chain Fv, VL | IMMUNE SYSTEM/APOPTOSIS |
| 6d94 | Peroxisome proliferator-activated | DNA BINDING PROTEIN/AGONIST |
| | receptor gamma | |
| 2axf | HLA class I histocompatibility | IMMUNE SYSTEM/GENE REGULATION |
| | antigen, B*3508 heavy chain | |
| 2bvp | HLA CLASS I | GLYCOPROTEIN/PEPTIDE |
| | HISTOCOMPATIBILITY | |
| | ANTIGEN, B-57 ALPHA CHAIN | |
| 2f5b | PROTEIN (ANTIBODY 2F5 | IMMUNOGLOBULIN |
| | (HEAVY CHAIN)) | |
| 1a1n | HLA class I histocompatibility | COMPLEX (ANTIGEN/PEPTIDE) |
| | antigen, BW-53 B*5301 alpha chain | |
| 1agb | B*0801 | HISTOCOMPATIBILITY COMPLEX |
| 1fgl | CYCLOPHILIN A | ISOMERASE/VIRAL PROTEIN |
| 2r7g | Retinoblastoma-associated protein | TRANSCRIPTION REPRESSOR, CELL |
| | | CYCLE |
| 1gux | RETINOBLASTOMA PROTEIN | COMPLEX (TRANSCRIPTION |
| | | REG/PEPTIDE) |
| 4o45 | WD repeat-containing protein 5 | TRANSCRIPTION/RNA BINDING PROTEIN |
| 4o42 | Chromodomain-helicase-DNA- | DNA BINDING PROTEIN/VIRAL PROTEIN |
| | binding protein 1 | |
| 1pyw | HLA class II histocompatibility | IMMUNE SYSTEM/PROTEIN |
| | antigen, DR alpha chain | BINDING/TOXIN |
| 1aot | FYN PROTEIN-TYROSINE | COMPLEX (PROTO-ONCOGENE/EARLY |
| | KINASE | PROTEIN |
| 4i5l | Serine/threonine-protein phosphatase | HYDROLASE/TOXIN |
| | 2A catalytic subunit alpha isoform, | |
| | PP2A-alpha | |
| 3w8j | Protein disulfide-isomerase A6 | OXIDOREDUCTASE/ISOMERASE |
| 5jiu | Ran-binding protein 9 | RAN BINDING PROTEIN/PEPTIDE |
| 2zjd | Microtubule-associated proteins | APOPTOSIS INHIBITOR/APOPTOSIS |
| | 1A/1B light chain 3B precursor | |
| 1l2i | ESTROGEN RECEPTOR | TRANSCRIPTION |
| | | RECEPTOR/COACTIVATOR |
| 4dma | Estrogen receptor | TRANSCRIPTION/PROTEIN |
| | | BINDING/AGONIST |
| 4lx2 | Unconventional myosin-Va | CONTRACTILE PROTEIN/PROTEIN |
| | | TRANSPORT |
| 4gxb | Sorting nexin-17 | PROTEIN TRANSPORT/CELL ADHESION |
| 5xxk | E3 ubiquitin-protein ligase Mdm2 | ONCOPROTEIN/INHIBITOR |
| 1thr | ALPHA-THROMBIN (LARGE | HYDROLASE(SERINE PROTEINASE) |
| | SUBUNIT) | |
| 1bd2 | HLA-A 0201 | COMPLEX (MHC/VIRAL |
| | | PEPTIDE/RECEPTOR) |
| 4e5x | HLA class I histocompatibility | IMMUNE SYSTEM/TRANSCRIPTION |
| | antigen, A-2 alpha chain | |
| 2kqf | Microtubule-associated | SIGNALING PROTEIN/PEPTIDE BINDING |
| | serine/threonine-protein kinase 2 | PRO |
| 1yvh | CBL E3 ubiquitin protein ligase | LIGASE, SIGNALING PROTEIN, IMMUNE |
| | | SYSTE |
| 2iv9 | AP-2 COMPLEX SUBUNIT BETA- | ENDOCYTOSIS/REGULATOR |
| | 2 | |
| 6mue | Calmodulin-1 | CALCIUM BINDING/TRANSPORT |
| | | PROTEIN |
| 4p9v | Growth factor receptor-bound protein | SIGNALING PROTEIN/ANTAGONIST |
| | 2 | |
| 5ttw | Polycomb protein EED | TRANSCRIPTION |
| | | REGULATOR/INHIBITOR |
| 6ckz | Caspase-3 subunit p12 | APOPTOSIS, HYDROLASE |
| 4q13 | Estrogen receptor | HORMONE BINDING PROTEIN/PEPTIDE |
| 5jmo | Furin | HYDROLASE/ANTIBODY |
| 4wyu | Protein scribble homolog | STRUCTURAL PROTEIN/PEPTIDE |
| 4xt9 | Nuclear receptor ROR-gamma | UNKNOWN PROTEIN |
| 5bwn | NAD-dependent protein deacetylase | PEPTIDE/HYDROLASE |
| | sirtuin-3, mitochondrial | |
| 5u1q | Growth factor receptor-bound protein | SIGNALING PROTEIN/PEPTIDE |
| | 7 | INHIBITOR |
| 4x1q | Urokinase-type plasminogen activator | HYDROLASE INHIBITOR/HYDROLASE |
| 2l3r | E3 ubiquitin-protein ligase UHRF1 | DNA BINDING PROTEIN/GENE |
| | | REGULATION |
| 5f8z | Plasma kallikrein LIGHT CHAIN | HYDROLASE-HYDROLASE INHIBITOR |
| 3ewt | Calmodulin | CALCIUM BINDING PROTEIN |
| 5vb9 | Interleukin-17A | IMMUNE SYSTEM/INHIBITOR |
| 1bck | PEPTIDYL-PROLYL CIS-TRANS | ISOMERASE/IMMUNOSUPPRESSANT |
| | ISOMERASE A | |
| 1ikf | IGG1-KAPPA R45-45-11 FAB | IMMUNE |
| | (HEAVY CHAIN) | SYSTEM/IMMUNOSUPPRESSANT |
| 2h9m | WD-repeat protein 5 | STRUCTURAL GENOMICS, GENE |
| | | REGULATION |
| 4U6Y | HLA-A | HLA-A HUMAN MHC I molecule |
| 6AT5 | HLA-B | HLA-B Human MHCI I molecule |
| 5VGE | HLA-C | HLA-C Human MHC I molecule |
| 1KPR | HLA-E | human non-classical major histocompatibility |
| | | complex molecule HLA-E |
| | HLA-F | human non-classical major histocompatibility |
| | | complex molecule HLA-F |
| 1YDP | HLA-G | human nonclassical MHC class I molecule |
| | | expressed at the fetal-maternal interface |
| 2IPK | HLA-DR | HLA -DR1 human MHC II |
| 3LQZ | HLA-DP | HLA -DP2 human MHC II |
| 5KSU | HLA-DQ | HLA-DQ2.5 human MHC II |
| 1I1A | Fc-Rn | neonatal Fc receptor that transports |
| | | immunoglobulin G across epithelia |
| 1ZAG | ZAG1 | Zinc alpha glycoprotein involved in lipid |
| | | degradation |
| | ULBP3 (NKG2D Ligands) | Cell surface glycoprotein natural ligand for |
| | | NKGD2 receptor |
| 1HYR | MICA | MHC class I homolog, a stress induced ligand |
| | | for NKGD2 receptor |
| 1JE6 | MICB | MHC class I homolog, a stress induced ligand |
| | | for NKGD2 receptor |
| 3D2U | UL18 (viral) | peptide binding viral MHC mimic |
| 4L4V | MR1 | MHC like molecle |
| 1A6Z | HFE | Hemochromatosis protein |
| 4FQX | HLA-DM | MHC chaperone protein |
| 1L8J | EPCR (Lipid presenting) | Endothelial Protein C receptor. Lipid presenting |
| | | MHC like molecule |
| 1ONQ | CD1 (Lipid presenting) | Cluster of Differentiation 1 gycoprotein |
| | | moleucle Lipid presenting MHC like molecule |

The following table provides information about the peptide known to bind to each human protein scaffold (i.e., “bound peptide”).

TABLE 3
Bound Peptide Information

| PDB | | | | | Bound |
|---|---|---|---|---|---|
| Entry | Protein Name | Bound Peptide | Bound Peptide | | Peptide |
| ID | (Protein Scaffold) | Sequence | SEQ ID NO: | Bound Peptide Description | Organism |
| 1cp3 | APOPAIN | DVADX | 311 | ACETYL-ASP-VAL-ALA-ASP- | — |
| | | | | FLUOROMETHYLKETONE (SEQ ID NO: | |
| | | | | 311) | |
| 1d5x | HLA CLASS II | XRXXX | 312 | DIPEPTIDE MIMETIC INHIBITOR | — |
| | HISTOCOMPATIBILI | | | | |
| | TY ANTIGEN | | | | |
| 1fch | PEROXISOMAL | YQSKL | 313 | PTS1-CONTAINING PEPTIDE | — |
| | TARGETING SIGNAL | | | | |
| | 1 RECEPTOR | | | | |
| 1g1g | PROTEIN TYROSINE | TDYXR | 314 | MONO-PHOSPHORYLATED PEPTIDE | — |
| | PHOSPHATASE 1B | | | FROM THE INSULIN RECEPTOR KINASE | |
| 1hcs | HUMAN SRC | XEEIE | 315 | ACETYL-PYEEIE-OH (SEQ ID NO: 315) | — |
| 1nop | tyrosyl-DNA | KLNYL | 316 | topoisomerase I-derived peptide | — |
| | phosphodiesterase 1 | | | | |
| 1nvq | Serine/threonine- | ASVSA | 317 | Peptide ASVSA (SEQ ID NO: 317) | — |
| | protein kinase Chk1 | | | | |
| 1pic | PHOSPHATIDYLINO | XVPML | 318 | BETA-PLATELET-DERIVED GROWTH | — |
| | SITOL 3-KINASE | | | FACTOR RECEPTOR | |
| 1twq | peptidoglycan | XAXKX | 319 | muramyl tripeptide | — |
| | recognition protein-I- | | | | |
| | alpha | | | | |
| 1tzs | Cathepsin E | PAMAM | 320 | 23-mer peptide from PelB-IgG kappa | — |
| | | | | light chain fusion protein | |
| 1vbs | CYCLOPHILIN A | AXPFX | 321 | TETRAPEPTIDE | — |
| 1vbt | CYCLOPHILIN A | AXPFX | 322 | SULFUR-SUBSTITUTED TETRAPEPTIDE | — |
| 1zys | Serine/threonine- | ASVSA | 323 | pentapeptide fragment | — |
| | protein kinase Chk1 | | | | |
| 2g99 | WD-repeat protein 5 | ARTXQ | 324 | Histone H3 | — |
| 2h13 | WD-repeat protein 5 | ARTXQ | 325 | histone H3 lys-4 dimethylated | — |
| 2m0u | Na(+)/H(+) exchange | QDTRL | 326 | C-terminal CFTR peptide | — |
| | regulatory cofactor | | | | |
| | NHE-RF1 | | | | |
| 2m0v | Na(+)/H(+) exchange | QDTRL | 327 | C-terminal CFTR peptide | — |
| | regulatory cofactor | | | | |
| | NHE-RF1 | | | | |
| 209k | WD repeat protein 5 | ARTXQ | 328 | H3 HISTONE | — |
| 2pxj | JmjC domain- | GGVXK | 329 | monomethylated Histone H3K36 peptide | — |
| | containing histone | | | | |
| | demethylation protein | | | | |
| | 3A | | | | |
| 2q5a | Peptidyl-prolyl cis- | FXXQX | 330 | Five residue peptide | — |
| | trans isomerase NIMA- | | | | |
| | interacting 1 | | | | |
| 3fvh | Serine/threonine- | LHSAX | 331 | Acetyl-Leu-His-Ser-phospho Thr-Ala-NH2 (SEQ | — |
| | protein kinase PLK1 | | | ID NO: 992) peptide | |
| 3h11 | Caspase-8 | AIETX | 332 | IETD aldehyde inhibitor | — |
| 3idi | 2F5 Fab light chain | ALDKW | 333 | gp41 MPER peptide | — |
| 3n7y | Growth factor receptor- | XVNVX | 334 | 20-membered peptide-like macrocyclic ligand | — |
| | bound protein 2 | | | | |
| 30a6 | Male-specific lethal 3 | HRXVL | 335 | H4 peptide monomethylated at lysine 20 | — |
| | homolog | | | | |
| 3rq7 | Serine/threonine- | PLXSX | 336 | C6H5(CH2)8-derivatized peptide inhibitor | — |
| | protein kinase PLK1 | | | | |
| 4dfw | Serine/threonine- | XLHSX | 337 | Peptide | — |
| | protein kinase PLK1 | | | | |
| 4dm9 | Ubiquitin carboxyl- | XVAXX | 338 | Tripeptide fluoromethyl ketone inhibitor Z- | — |
| | terminal hydrolase | | | VAE(OMe)-FMK | |
| | isozyme L1 | | | | |
| 4gm9 | WD repeat-containing | XXRXX | 339 | MM-401 | — |
| | protein 5 | | | | |
| 4iim | Intersectin-1 | VMGPW | 340 | peptide ligand | — |
| 4joh | Golgi-associated PDZ | PTSII | 341 | H-iCAL36 peptide | — |
| | and coiled-coil motif- | | | | |
| | containing protein | | | | |
| 4jr0 | Procaspase-3 | DEVXX | 342 | ACE-DEVD-CMK* (SEQ ID NO: 342) | — |
| | | | | *CMK: chloromethylketone | |
| 1a7c | PLASMINOGEN | TVASSX | 343 | PENTAPEPTIDE | — |
| | ACTIVATOR | | | | |
| | INHIBITOR TYPE 1 | | | | |
| 1awq | CYCLOPHILIN A | HAGPIA | 344 | PEPTIDE FROM THE HIV-1 CAPSID | — |
| | | | | PROTEIN | |
| 1bm2 | PROTEIN (GROWTH | XXVNVP | 345 | PROTEIN (PKF273-791) | — |
| | FACTOR RECEPTOR | | | | |
| | BOUND PROTEIN 2) | | | | |
| 1f8h | EPIDERMAL | STNPFR | 346 | PTGSSSTNPFR (SEQ ID NO: 998) | — |
| | GROWTH FACTOR | | | | |
| | RECEPTOR | | | | |
| | SUBSTRATE 15 | | | | |
| 1ir3 | INSULIN RECEPTOR | GDYMNM | 347 | PEPTIDE SUBSTRATE | — |
| 1ywt | 14-3-3 protein sigma | SHXYPA | 348 | synthetic optimal phosphopeptide (mode-1) | — |
| 2oqs | Disks large homolog 1 | RRETQV | 349 | C-terminal HPV-18 E6 peptide | — |
| 2pie | E3 ubiquitin-protein | ELKERY | 350 | phosphopeptide | — |
| | ligase RNF8 | | | | |
| 3kqi | PHD finger protein 2 | ARTXQT | 351 | H3K4Me3 peptide | — |
| 3tkz | Tyrosine-protein | VIXFVP | 352 | PROTEIN (RVIpYFVPLNR (SEQ ID NO: 999) | — |
| | phosphatase non- | | | peptide) | |
| | receptor type 11 | | | | |
| 3v40 | Mucosa-associated | XVRPRX | 353 | MALT1 Inhibitor | — |
| | lymphoid tissue | | | | |
| | lymphoma | | | | |
| | translocation protein 1 | | | | |
| 4jdi | Serine/threonine- | RRRRSW | 354 | Paktide S | — |
| | protein kinase PAK 4 | | | | |
| 4nmp | Golgi-associated PDZ | WPXSII | 355 | iCAL36(Ac-K-3) peptide | — |
| | and coiled-coil motif- | | | | |
| | containing protein | | | | |
| 4nnm | Tax1-binding protein 3 | YPTSII | 356 | TIP-1 PDZ domain | — |
| 4q6h | CFTR-associated | VQDTRL | 357 | iCAL36-VQDTRL peptide | — |
| | ligand | | | | |
| 1f8a | PEPTIDYL-PROLYL | YXPTXPS | 358 | Y(SEP)*PT(SEP)S PEPTIDE (SEQ ID NO: | — |
| | CIS-TRANS | | | 1011) | |
| | ISOMERASE NIMA- | | | *SEP: phosphoserine | |
| | INTERACTING 1 | | | | |
| 117z | CALMODULIN | GGKLSKX | 359 | CAP-23/NAP-22 | — |
| 2gfa | Jumonji domain- | ARTXQTA | 360 | peptide | — |
| | containing protein 2A | | | | |
| 2ovr | F-box/WD repeat | SLIPPDK | 361 | cyclinE N-terminal degron | — |
| | protein 7 | | | | |
| 3jpv | Proto-oncogene | KRRRHPS | 362 | Peptide (PIMTIDE) ARKRRRHPSGPPTA (SEQ | — |
| | serine/threonine-protein | | | ID NO: 1012) | |
| | kinase Pim-1 | | | | |
| 4E35 | Golgi-associated PDZ | RWPTSIL | 363 | iCAL50 peptide | — |
| | and coiled-coil motif- | | | | |
| | containing protein | | | | |
| 4po2 | Heat shock 70 kDa | NRLLLTG | 364 | HSP70 substrate peptide | — |
| | protein 1A/1B | | | | |
| 1cwd | P56LCK TYROSINE | EGDXEEVL | 365 | (PHOSPHONOMETHYL)PHENYLALANINE- | — |
| | KINASE | | | CONTAINING PEPTIDE PRO-GLU-GLY- | |
| | | | | ASP-PM3-GLU-GLU-VAL-LEU- (SEQ ID NO: | |
| | | | | 1013) | |
| 2qbw | PDZ-Fibronectin fusion | PQPVDSWV | 366 | Polypeptide | — |
| | protein | | | | |
| 3ch8 | fusion protein PDZ- | PQPVDSWV | 367 | C-terminal octapeptide from protein ARVCF | — |
| | Fibronectin | | | (SEQ ID NO: 989) | |
| 3hna | Histone-lysine N- | KQTARXST | 368 | Mono-Methylated H3K9 Peptide | — |
| | methyltransferase, H3 | | | | |
| | lysine-9 specific 5 | | | | |
| 4e3b | Tax1-binding protein 3 | SRWPTSIL | 369 | iCAL50 peptide | — |
| 4okw | Androgen receptor | DTLFSQHY | 370 | co-regulator peptide | — |
| 1a9e | HLA CLASS I | LPPLDITPY | 371 | PEPTIDE LPPLDITPY (SEQ ID NO: 371) | — |
| | HISTOCOMPATIBILI | | | | |
| | TY ANTIGEN, B-35 | | | | |
| | B*3501 (ALPHA | | | | |
| | CHAIN) | | | | |
| 1cf0 | PROTEIN (PROFILIN) | PPPPPPPP(IYR)* | 372 | PROTEIN (L-PRO10-IODOTYROSINE (SEQ | — |
| | | *IYR: 3-IODO- | | ID NO: 1002)) | |
| | | TYROSINE | | | |
| 1eeo | PROTEIN TYROSINE | LEFXMDYEX | 373 | ACETYL-E-L-E-F-PTR-M-D-Y-E-NH2 | — |
| | PHOSPHATASE 1B | | | PEPTIDE (SEQ ID NO: 1003) | |
| 1hsa | CLASS I | ARAAAAAAA | 374 | MODEL PEPTIDE SEQUENCE - | — |
| | HISTOCOMPATIBILI | | | ARAAAAAAA (SEQ ID NO: 374) | |
| | TY ANTIGEN (HLA- | | | | |
| | B*2705) | | | | |
| 3fe7 | Mdm4 protein | FMXXWEXLX | 375 | p53-peptidomimetic (Ac-Phe-Met-Aib *- Pmp | — |
| | | | | **- Trp-Glu-Ac3c ***- Leu-NH2) | |
| | | | | *Aib: ALPHA-AMINOISOBUTYRIC ACID | |
| | | | | ** Pmp: 4'-DEOXY-4'-AMINOPYRIDOXAL-5'- | |
| | | | | PHOSPHATE | |
| | | | | Ac3c: 1- | |
| | | | | AMINOCYCLOPROPANECARBOXYLIC | |
| | | | | ACID | |
| 3kpq | HLA class I | EEYLKAWTF | 376 | EEYLKAWTF (SEQ ID NO: 376), mimotope | — |
| | histocompatibility | | | peptide | |
| | antigen, B-44 alpha | | | | |
| | chain | | | | |
| 4jij | Matrix | XPLGXXARX | 377 | fluorogenic peptidic substrate | — |
| | metalloproteinase-9 | | | (8MC)*PLG(PHI) ** (DNW) *** AR(NH2) (SEQ | |
| | | | | ID NO: 377) | |
| | | | | *8MC: (7-methoxy-2-oxo-2H-chromen-4- | |
| | | | | yl)acetic acid; | |
| | | | | ** PHI: Iodo-phenylalanine; | |
| | | | | DNW: 3-[(2,4-dinitrophenyl)amino]-L- | |
| | | | | alanine | |
| 1aqc | X11 | GYENPTYKFF | 378 | PEPTIDE | — |
| 2axi | Ubiquitin-protein ligase | PFEXLDWEFX | 379 | cyclic 8-mer peptide | — |
| | E3 Mdm2 | | | | |
| 2002 | 14-3-3 protein | GLLDALDLAS | 380 | ExoS (416-430) peptide | — |
| | zeta/delta | | | | |
| 3p9g | Tumor susceptibility | PEXTAPPEEX | 381 | Gag polyprotein | — |
| | gene 101 protein | | | | |
| 3qhr | Cell division protein | PKTPKKAKKL | 382 | CDK2 substrate peptide: PKTPKKAKKL (SEQ | — |
| | kinase 2 | | | ID NO: 382) | |
| 3sfj | Tax1-binding protein 3 | ANSRWPTSII | 383 | decameric peptide iCAL36 | — |
| 4j9c | Tyrosine-protein kinase | APTYSPPLPP | 384 | P17 | — |
| | ABL1 | | | | |
| 4rxz | Protein Mdm4 | TSFAEYWNLL | 385 | 12-MER PEPTIDE INHIBITOR | — |
| 1ov3 | Neutrophil cytosol | QPPSNPPPRPP | 386 | Flavocytochrome b558 alpha polypeptide | — |
| | factor 1 | | | | |
| 2p81 | nmAb 2F5, light chain | ELLELDKWASL | 387 | gp41 peptide | — |
| 3p72 | Platelet glycoprotein Ib | CTERMALHNL | 388 | OS1 peptide | — |
| | alpha chain | C | | | |
| 1y98 | Breast cancer type 1 | PTRVSXPVFGA | 389 | CtIP PHOSPHORYLATED PEPTIDE | — |
| | susceptibility protein | T | | | |
| 21gk | E3 ubiquitin-protein | ARTKQTARXST | 390 | histone H3 peptide | — |
| | ligase UHRF1 | G | | | |
| 3eqy | Mdm4 protein | TSFAEYWNLLS | 39 | 12-mer peptide inhibitor | — |
| | | P | | | |
| 20j2 | Hematopoetic Cell | HSKYPLPPLPSL | 392 | artificial peptide PD1 | — |
| | Kinase, SH3 domain | X | | | |
| 3d8c | Hypoxia-inducible | QLTSYDCEVN | 393 | HYPOXIA-INDUCIBLE FACTOR 1 ALPHA | — |
| | factor 1 alpha inhibitor | API | | | |
| 2qki | Complement C3 | ICVWQDWGAH | 394 | compstatin | — |
| | | RCTX | | | |
| 4fmq | Mitogen-activated | LSSLAASSLAK | 395 | MAPK DOCKING PEPTIDE | — |
| | protein kinase 1 | RRQQ | | | |
| 2m3m | Disks large homolog 1 | XRTRORNETQ | 396 | Protein E6 | Alphapapillo |
| | | V | | | mavirus 5 |
| 5ic3 | Golgi-associated PDZ | RRETQV | 397 | HPV18E6 Peptide | Alphapapillo |
| | and coiled-coil motif- | | | | mavirus 7 |
| | containing protein | | | | |
| 2kpl | Membrane-associated | RSSRTRRETQV | 398 | Protein E6 | Alphapapillo |
| | guanylate kinase, WW | | | | mavirus 9 |
| | and PDZ domain- | | | | |
| | containing protein 1 | | | | |
| 4pz5 | Fibronectin | SISYTDEIEEED | 399 | Fibronectin-binding protein BBK32 | Borreliella |
| | | YD | | | burgdorferi |
| 217u | Advanced | DEFXADE | 400 | Serum albumin peptide | Bos taurus |
| | glycosylation end | | | | |
| | product-specific | | | | |
| | receptor | | | | |
| 6hy7 | Neuronal acetylcholine | GCCSDPRCRYR | 401 | Alpha-conotoxin RgIA | Conus regius |
| | receptor subunit alpha- | CX | | | |
| | 9 | | | | |
| 4lcy | HLA class I | FTMRLLSPV | 402 | dengue l poly peptide | Dengue virus |
| | histocompatibility | | | | |
| | antigen, B-46 alpha | | | | |
| | chain | | | | |
| 3hql | Speckle-type POZ | DEVTSTT | 403 | Puckered | Drosophila |
| | protein | | | | melanogaster |
| 3hqm | Speckle-type POZ | PDVSSST | 404 | Protein cubitus interruptus | Drosophila |
| | protein | | | | melanogaster |
| 2ff6 | Gelsolin | ETNEKNPLPDK | 405 | CG4944-PC, isoform C | Drosophila |
| | | | | | melanogaster |
| 3f20 | SPRY domain- | DINNNNNIVED | 406 | 20-mer peptide from ATP-dependent RNA | Drosophila |
| | containing SOCS box | VERK | | helicase vasa | melanogaster |
| | protein 1 | | | | |
| 2cia | CYTOPLASMIC | HIXDEVAAD | 407 | TRANSLOCATED INTIMIN RECEPTOR | Escherichia |
| | PROTEIN NCK2 | | | | coli |
| 5h7g | B-cell lymphoma 6 | LWYTDIRMSW | 408 | F1324 peptide | Escherichia |
| | protein | RVP | | | virus T7 |
| 5h5q | Phospholipid | CRVDLQGWRR | 409 | GXpep-1 | Escherichia |
| | hydroperoxide | CRRX | | | virus T7 |
| | glutathione peroxidase, | | | | |
| | mitochondrial | | | | |
| 1bt6 | S100A10 | STVHEILSKLS | 410 | ANNEXIN II | Gallus gallus |
| 6d3z | Plasminogen | GRCYKSRPPIC | 411 | Trypsin inhibitor 1 | Helianthus |
| | | FPN | | | annuus |
| 3iec | Serine/threonine- | FPLKRHDKVD | 412 | Cytotoxicity-associated immunodominant | Helicobacter |
| | protein kinase MARK2 | DLSK | | antigen | pylori |
| 3ua7 | Tyrosine-protein kinase | APPIPPPR | 413 | Non-structural protein 5A | Hepacivirus C |
| | Fyn | | | | |
| 4j1v | MOB kinase activator | ALPAWARPD | 414 | NS5A domain II peptide | Hepacivirus C |
| | 1A | | | | |
| 5jzi | HLA class I | KLVALGINAV | 415 | KLV peptide | Hepacivirus C |
| | histocompatibility | | | | |
| | antigen, A-2 alpha | | | | |
| | chain | | | | |
| 4jzo | Anti-HCV E2 Fab | GWLAGLFYQH | 416 | Envelope glycoprotein E2 | Hepacivirus C |
| | HC84-27 light chain | K | | | |
| 5kzp | HCV1-C1 Antibody | CQLINTNGSWH | 417 | C1 Epitope | Hepacivirus C |
| | Fab Light Chain | INCK | | | |
| 3zhf | AP-1 COMPLEX | PDWDFNX | 418 | LARGE ENVELOPE PROTEIN | Hepatitis B |
| | SUBUNIT GAMMA- | | | | virus |
| | LIKE 2 | | | | |
| 11hc | ALPHA-THROMBIN | GDFEEIP | 419 | HIRUDIN | Hirudo |
| | | | | | medicinalis |
| 3d49 | Thrombin heavy chain | DFEEIPEEX | 420 | Hirudin variant-1 | Hirudo |
| | | | | | medicinalis |
| 1ai8 | ALPHA-THROMBIN | DFEEIPEEXL | 421 | HIRUDIN IIIB | Hirudo |
| | (LARGE SUBUNIT) | | | | medicinalis |
| 1hah | ALPHA-THROMBIN | DFEEIPEEXL | 422 | HIRUGEN | Hirudo |
| | (LARGE SUBUNIT) | | | | medicinalis |
| 1hgt | ALPHA-THROMBIN | DFEEIPEEXL | 423 | HIRUGEN | Hirudo |
| | (LARGE SUBUNIT) | | | | medicinalis |
| 1hxe | THROMBIN | DFEEIPGEYL | 424 | HIRUDIN VARIANT-1 | Hirudo |
| | | | | | medicinalis |
| 1zrb | thrombin | DFEEIPEEXL | 425 | 11-peptide hirudin fragment | Hirudo |
| | | | | | medicinalis |
| 1mue | THROMBIN | DFEEIPEEXLQ | 426 | HIRUDIN IIB | Hirudo |
| | | | | | medicinalis |
| 1bhx | ALPHA THROMBIN | DFEEI | 427 | ALPHA THROMBIN | Homo sapiens |
| 1h90 | PHOSPHATIDYLINO | XVPML | 428 | BETA-PLATELET-DERIVED GROWTH | Homo sapiens |
| | SITOL 3-KINASE | | | FACTOR RECEPTOR | |
| 2aij | Sulfatase modifying | CTPSR | 429 | CTPSR peptide from Arylsulfatase A | Homo sapiens |
| | factor 1 | | | | |
| 2ce9 | TRANSDUCIN-LIKE | MWRPW | 430 | WRPW PEPTIDE (SEQ ID NO: 1010) | Homo sapiens |
| | ENHANCER | | | | |
| | PROTEIN 1 | | | | |
| 2hi8 | Sulfatase-modifying | CTPSR | 431 | CTPSR (SEQ ID NO: 429) | Homo sapiens |
| | factor 1 | | | | |
| 3cbo | Histone-lysine N- | KRSXK | 432 | Estrogen receptor | Homo sapiens |
| | methyltransferase | | | | |
| | SETD7 | | | | |
| 3E50 | Insulin-degrading | VVSHF | 433 | Protransforming growth factor alpha | Homo sapiens |
| | enzyme | | | | |
| 3k2u | Hepatocyte growth | ACGRR | 434 | Hepatocyte growth factor activator | Homo sapiens |
| | factor activator long | | | short chain | |
| | chain | | | | |
| 3035 | Transcription | ARXSA | 435 | Histone H3.1 | Homo sapiens |
| | intermediary factor 1- | | | | |
| | alpha | | | | |
| 3qj6 | Hepatoma-derived | QDFXT | 436 | H3K79me3 | Homo sapiens |
| | growth factor-related | | | | |
| | protein 2 | | | | |
| 3rbq | Protein unc-119 | XGAGA | 437 | Guanine nucleotide-binding protein G(t) | Homo sapiens |
| | homolog A | | | subunit alpha-1 | |
| 3r17 | Disks large homolog 1 | YLVTS | 438 | 11-mer peptide from Adenomatous polyposis | Homo sapiens |
| | | | | coli protein | |
| 3vro | Signal transduction | NEXTA | 439 | Proto-oncogene tyrosine-protein kinase Src | Homo sapiens |
| | protein CBL-C | | | | |
| 3zdy | INTEGRIN BETA-3 | GRGDS | 440 | RGD PEPTIDE | Homo sapiens |
| 3zvy | E3 UBIQUITIN- | ARTKQ | 441 | HISTONE H3.1 | Homo sapiens |
| | PROTEIN LIGASE | | | | |
| | UHRF1 | | | | |
| 4n3w | CREB-binding protein | RHRXV | 442 | Histone H4 peptide | Homo sapiens |
| 4ny3 | Serine/threonine- | PDYFL | 443 | Serine/threonine-protein phosphatase 2A | Homo sapiens |
| | protein phosphatase 2A | | | catalytic subunit alpha isoform | |
| | activator | | | | |
| 4ztd | Proliferating cell | AGAGA | 444 | ALA-GLY-ALA-GLY-ALA (SEQ ID NO: 444) | Homo sapiens |
| | nuclear antigen | | | | |
| 5c27 | Tyrosine-protein kinase | EVYES | 445 | GLU-VAL-TYR-GLU-SER (SEQ ID NO: 445) | Homo sapiens |
| | SYK | | | | |
| 5ulo | 14-3-3 protein | SPXFX | 446 | TBC1 domain family member 7 | Homo sapiens |
| | zeta/delta | | | | |
| 6bfj | Caspase-3 | DEVDX | 447 | Ac-Asp-Glu-Val-Asp-CMK* | Homo sapiens |
| | | | | (SEQ ID NO: 447) | |
| | | | | *CMK: chloromethylketone | |
| 6dn6 | SPRY domain- | INNNX | 448 | INNN(ABU)* (SEQ ID NO: 448) cyclic peptide | Homo sapiens |
| | containing SOCS box | | | inhibitor | |
| | protein 2 | | | *ABU: gamma-amino-butanoic acid | |
| 6j8f | Tubulinyl-Tyr | EGECY | 449 | 8-mer peptide | Homo sapiens |
| | carboxypeptidase 1 | | | | |
| 1csy | SYK PROTEIN | TXETLX | 450 | ACETYL-THR-PTR-GLU-THR-LEU-NH2 | Homo sapiens |
| | TYROSINE KINASE | | | (SEQ ID NO: 450) | |
| 1tw6 | Baculoviral IAP repeat- | AVPIAQ | 451 | Diablo homolog, mitochondrial | Homo sapiens |
| | containing protein 7 | | | | |
| 2ain | Afadin | LFSTEV | 452 | 6-mer peptide from Breakpoint cluster region | Homo sapiens |
| | | | | protein | |
| 2br9 | 14-3-3 PROTEIN | RQRXAP | 453 | CONSENSUS PEPTIDE FOR 14-3-3 | Homo sapiens |
| | EPSILON | | | PROTEINS | |
| 2eax | Peptidoglycan | AXKXXX | 454 | GLYCOSAMYL MURAMYL PENTAPEPTIDE | Homo sapiens |
| | recognition protein-I- | | | | |
| | beta | | | | |
| 2ri7 | Nucleosome- | ARTXQT | 455 | histone H3.1 | Homo sapiens |
| | remodeling factor | | | | |
| | subunit BPTF | | | | |
| 3iqu | 14-3-3 protein sigma | QRSTXT | 456 | 6-mer from RAF proto-oncogene | Homo sapiens |
| | | | | serine/threonine-protein kinase | |
| 3lny | Tyrosine-protein | EQVSAV | 457 | Rap guanine nucleotide exchange factor 6 | Homo sapiens |
| | phosphatase non- | | | | |
| | receptor type 13 | | | | |
| 3u4s | Lysine-specific | ARXSCG | 458 | HISTONE 3 TAIL ANALOG (T11C Peptide) | Homo sapiens |
| | demethylase 4A | | | | |
| 4bve | NAD-DEPENDENT | SGXVMR | 459 | ACETYL-COENZYME A SYNTHETASE 2- | Homo sapiens |
| | PROTEIN | | | LIKE, MITOCHONDRIAL | |
| | DEACETYLASE | | | | |
| | SIRTUIN-3, | | | | |
| | MITOCHONDRIAL | | | | |
| 4fr3 | 14-3-3 protein sigma | KRRKXV | 460 | TASK-3 peptide | Homo sapiens |
| 4hix | Humanized 3D6 Fab | DAEFRH | 461 | Beta-amyloid protein 40 | Homo sapiens |
| | light chain | | | | |
| 4062 | Zinc finger CW-type | ARTXQT | 462 | Histone H3.3 | Homo sapiens |
| | PWWP domain protein | | | | |
| | 2 | | | | |
| 4pxm | Estrogen receptor | ILHRLL | 463 | Nuclear receptor coactivator 2 | Homo sapiens |
| 4rh5 | Tyrosine-protein | FSAXPS | 464 | Epidermal growth factor receptor | Homo sapiens |
| | phosphatase non- | | | substrate 15 | |
| | receptor type 3 | | | | |
| 4wk2 | Integrin beta-1 | GRGDSP | 465 | GLY-ARG-GLY-ASP-SER-PRO (SEQ ID NO: | Homo sapiens |
| | | | | 465) | |
| 5eg2 | Histone-lysine N- | SKSXDR | 466 | Transcription initiation factor TFIID | Homo sapiens |
| | methyltransferase | | | subunit 10 | |
| | SETD7 | | | | |
| 5f2u | Retinal rod rhodopsin- | SSTIXX | 467 | Phosphatidylinositol 4,5-bisphosphate 5- | Homo sapiens |
| | sensitive cGMP 3',5'- | | | phosphatase, s-farnesyl-l-cysteine | |
| | cyclic | | | methyl ester | |
| | phosphodiesterase | | | | |
| | subunit delta | | | | |
| 5ggp | Protein O-linked- | TPTPVX | 468 | 10-mer Peptide from Dystroglycan | Homo sapiens |
| | mannose beta-1,2-N- | | | | |
| | acetylglucosaminyltran | | | | |
| | sferase 1 | | | | |
| 5lso | Splicing factor 45 | KSRWDE | 469 | LYS-SER-ARG-TRP-ASP-GLU (SEQ ID NO: | Homo sapiens |
| | | | | 469) | |
| 5xup | Telomeric repeat- | ILLTPR | 470 | Telomere repeats-binding bouquet formation | Homo sapiens |
| | binding factor 1 | | | protein 1 | |
| 1ca9 | PROTEIN (TNF | PFSKEEC | 471 | PROTEIN (TNF-R2) | Homo sapiens |
| | RECEPTOR | | | | |
| | ASSOCIATED | | | | |
| | FACTOR 2) | | | | |
| 1qjb | 14-3-3 PROTEIN | RSHXYPA | 472 | PHOSPHOPEPTIDE | Homo sapiens |
| | ZETA/DELTA | | | | |
| 1ujk | ADP-ribosylation | DDIXLLK | 473 | C-terminal peptide from Beta-secretase | Homo sapiens |
| | factor binding protein | | | | |
| | GGA1 | | | | |
| 3bun | E3 ubiquitin-protein | NDXIDNP | 474 | 13-meric peptide from Portein sprouty homolog | Homo sapiens |
| | ligase CBL | | | 4 | |
| 3hqu | Egl nine homolog 1 | MDDDFQL | 475 | Hypoxia-inducible factor 1 alpha | Homo sapiens |
| 3qlc | Transcriptional | ARTKQTA | 476 | peptide of Histone H3.3 | Homo sapiens |
| | regulator ATRX | | | | |
| 3shb | E3 ubiquitin-protein | ARTKQTA | 477 | Histone H3 peptide | Homo sapiens |
| | ligase UHRF1 | | | | |
| 3sov | Low-density | LPNAIGR | 478 | Sclerostin | Homo sapiens |
| | lipoprotein receptor- | | | | |
| | related protein 6 | | | | |
| 3v43 | Histone | ARTKQTA | 479 | Histone H3.1 | Homo sapiens |
| | acetyltransferase | | | | |
| | KAT6A | | | | |
| 4es0 | WD repeat-containing | GCARSEG | 480 | Histone-lysine N-methyltransferase SETD1B | Homo sapiens |
| | protein 5 | | | | |
| 411u | RNA polymerase- | GSRPMYG | 481 | Transcription elongation factor SPT5 | Homo sapiens |
| | associated protein | | | | |
| | RTF1 homolog | | | | |
| 417x | Death-inducer | ARTXQTA | 482 | Histone H3 peptide | Homo sapiens |
| | obliterator 1 | | | | |
| 4mvk | Neutrophil gelatinase- | VFFAEDX | 483 | Amyloid peptide fragment VFFAED (SEQ ID | Homo sapiens |
| | associated lipocalin | | | NO: 1006) | |
| 6jwm | SPRY domain- | RGDINNN | 484 | Nitric oxide synthase, inducible | Homo sapiens |
| | containing SOCS box | | | | |
| | protein 2 | | | | |
| 1gjb | UROKINASE-TYPE | LKFQCGQK | 48.5 | UROKINASE-TYPE PLASMINOGEN | Homo sapiens |
| | PLASMINOGEN | | | ACTIVATOR | |
| | ACTIVATOR | | | | |
| 2blz | Estrogen receptor | KILHRLLQ | 486 | Nuclear receptor coactivator 2 | Homo sapiens |
| 2kbs | Harmonin | TPLEITEL | 487 | octameric peptide from Cadherin-23 | Homo sapiens |
| 2rt5 | Msx2-interacting | YETLXDXE | 488 | peptide from Silencing mediator of retinoic | Homo sapiens |
| | protein | | | acid and thyroid hormone receptor | |
| 2w30 | BIFUNCTIONAL | YAGXDENX | 489 | DNA REPAIR PROTEIN XRCCI | Homo sapiens |
| | POLYNUCLEOTIDE | | | | |
| | PHOSPHATASE/KIN | | | | |
| | ASE | | | | |
| 3t03 | Peroxisome | HKILHRLL | 490 | Nuclear receptor coactivator 1 | Homo sapiens |
| | proliferator-activated | | | | |
| | receptor gamma | | | | |
| 4bxf | BIFUNCTIONAL | GCLHHHRI | 491 | 60S RIBOSOMAL PROTEIN L27A | Homo sapiens |
| | LYSINE-SPECIFIC | | | | |
| | DEMETHYLASE | | | | |
| | AND HISTIDYL- | | | | |
| | HYDROXYLASE | | | | |
| | MINA | | | | |
| 4hrg | Protein S100-A10 | MKIPKFTF | 492 | Neuroblast differentiation-associated protein | Homo sapiens |
| | | | | AHNAK | |
| 41xl | Lysine-specific | ARXSTGGK | 493 | H3 peptide | Homo sapiens |
| | demethylase 4B | | | | |
| 6e3g | Nuclear receptor ROR- | KILHRLLQ | 494 | co-activator peptide | Homo sapiens |
| | gamma | | | | |
| 6uyz | Small ubiquitin-related | AEERIIVL | 495 | phosphorylated DAXX | Homo sapiens |
| | modifier 1 | | | | |
| 1gwq | OESTROGEN | KILHRLLQD | 496 | NUCLEAR RECEPTOR COACTIVATOR 2 | Homo sapiens |
| | RECEPTOR | | | | |
| 1lck | P56==LCK== | EGQXQPQPA | 497 | TAIL PHOSPHOPEPTIDE | Homo sapiens |
| | TYROSINE KINASE | | | TEGQ(PHOSPHO)YQPQPA (SEQ ID NO: | |
| | | | | 1004) | |
| 1oga | T-CELL RECEPTOR | GILGFVFTL | 498 | GILGFVFTL (SEQ ID NO: 498) | Homo sapiens |
| | BETA CHAIN C | | | | |
| | REGION | | | | |
| 1qew | PROTEIN (HLA | FLWGPRALV | 499 | PROTEIN (MELANOMA-ASSOCIATED | Homo sapiens |
| | CLASS I | | | ANTIGEN 3) | |
| | HISTOCOMPATIBILI | | | | |
| | TY ANTIGEN, B-35 | | | | |
| | B* 3501 ALPHA | | | | |
| | CHAIN) | | | | |
| 1xr9 | HLA class I | ILGPPGSVY | 500 | Ubiquitin-conjugating enzyme E2 E1 | Homo sapiens |
| | histocompatibility | | | | |
| | antigen, B-15 alpha | | | | |
| | chain | | | | |
| 2cbl | PROTO-ONCOGENE | SDGXTPEPA | 501 | ZAP-70 | Homo sapiens |
| | CBL | | | | |
| 2dlx | cortactin isoform a | KRPPPPPPG | 502 | proline rich region from development and | Homo sapiens |
| | | | | differentiation enhancing factor 1 | |
| 2y ln | E3 UBIQUITIN- | SDGXTPEPA | 503 | TYROSINE-PROTEIN KINASE ZAP-70 ZAP- | Homo sapiens |
| | PROTEIN LIGASE | | | 70,70 KDA ZETA-ASSOCIATED PROTEIN, | |
| | | | | SYK-RELATED TYROSINE KINASE | |
| 3al3 | DNA topoisomerase 2- | SIYFPELYD | 504 | Peptide of Fanconi anemia group J protein | Homo sapiens |
| | binding protein 1 | | | | |
| 3uu7 | Estrogen receptor | KILHRLLQE | 505 | Nuclear receptor coactivator 1 | Homo sapiens |
| 3uzd | 14-3-3 protein gamma | SPXLPNITL | 506 | Histone deacetylase 4 | Homo sapiens |
| 4ch9 | KELCH-LIKE | EPEEPEADQ | 507 | SERINE/THREONINE-PROTEIN KINASE | Homo sapiens |
| | PROTEIN 3 | | | WNK4 | |
| 4jy | Retinoic acid receptor | HKILHRLLQ | 508 | Nuclear receptor coactivator 1 | Homo sapiens |
| | beta | | | | |
| 4nnx | HLA class I | RQAXLSISV | 509 | Serine/threonine-protein kinase D2 | Homo sapiens |
| | histocompatibility | | | | |
| | antigen, A-2 alpha | | | | |
| | chain | | | | |
| 5E50 | Aprataxin and PNK- | AYDEXDEEX | 510 | XRCC4 | Homo sapiens |
| | like factor | | | | |
| 6ivx | Nuclear receptor ROR- | EAIIRKALM | 511 | Nuclear receptor corepressor 2 | Homo sapiens |
| | gamma | | | | |
| 1mw4 | Growth factor receptor- | PQPEXVNQPD | 512 | Receptor protein-tyrosine kinase erbB-2 | Homo sapiens |
| | bound protein 7 | | | | |
| 1om9 | ADP-ribosylation | DFGGFEAAET | 513 | 15-mer peptide fragment of p56 | Homo sapiens |
| | factor binding protein | | | | |
| | GGA1 | | | | |
| 209v | Ponsin | VPPPVPPPPS | 514 | Paxillin | Homo sapiens |
| 2q7i | Androgen receptor | GAFQNLFQSV | 515 | Androgen receptor | Homo sapiens |
| 2r01 | Hepatocyte growth | PGRQACGRRH | 516 | Hepatocyte growth factor activator | Homo sapiens |
| | factor activator | | | | |
| 2rr4 | Zinc finger CW-type | ARTXQTARKS | 517 | Histone H3 | Homo sapiens |
| | PWWP domain protein | | | | |
| | 1 | | | | |
| 3oll | Estrogen receptor beta | RHKILHRLLQ | 518 | Nuclear receptor coactivator 1 | Homo sapiens |
| 3pfv | E3 ubiquitin-protein | LQRXSSDPTG | 519 | 11-meric peptide from Epidermal growth factor | Homo sapiens |
| | ligase CBL-B | | | receptor | |
| 3qhe | Adenomatous polyposis | SYEGYEGYYS | 520 | KH domain-containing, RNA-binding, signal | Homo sapiens |
| | coli protein | | | transduction-associated protein 1 | |
| 3zke | DYNEIN LIGHT | GMHSKGTQTA | 521 | NEK9 PROTEIN | Homo sapiens |
| | CHAIN 1, | | | | |
| | CYTOPLASMIC | | | | |
| 4htp | DNA ligase 4 | DVPQWEVFFK | 522 | Protein artemis | Homo sapiens |
| 4kmd | Sufu | GSYGHLSIGT | 523 | Zinc finger protein GLII | Homo sapiens |
| 4n39 | UDP-N- | ETGTTNTATT | 524 | Host cell factor 1 | Homo sapiens |
| | acetylglucosamine -- | | | | |
| | peptide N- | | | | |
| | acetylglucosaminyltran | | | | |
| | sferase 110 kDa subunit | | | | |
| 4y29 | Peroxisome | KSLLQQLLTE | 525 | Peptide from Nuclear receptor coactivator 1 | Homo sapiens |
| | proliferator-activated | | | | |
| | receptor gamma | | | | |
| 4yje | Adenomatous polyposis | LTGCGDIIAE | 526 | APC membrane recruitment protein 1 | Homo sapiens |
| | coli protein | | | | |
| 5glf | Transitional | HNWGQGFRLG | 527 | Derlin-1 | Homo sapiens |
| | endoplasmic reticulum | | | | |
| | ATPase | | | | |
| 5h43 | Importin subunit alpha- | RNQKRKHDEI | 528 | Histone acetyltransferase KAT8 | Homo sapiens |
| | 1 | | | | |
| 5ni8 | Nuclear receptor ROR- | KILHRLLQDS | 529 | The Tethered SRC2-2 peptide | Homo sapiens |
| | gamma | | | | |
| 5y49 | Bile acid receptor | ENLLLRYLLD | 530 | Peptide from Nuclear receptor coactivator 2 | Homo sapiens |
| 112z | CD2 ANTIGEN | SHRPPPPGHRV | 531 | T-CELL SURFACE ANTIGEN CD2 | Homo sapiens |
| | (CYTOPLASMIC | | | | |
| | TAIL)-BINDING | | | | |
| | PROTEIN 2 | | | | |
| 1w70 | NEUTROPHIL | KPQPAVPPRPS | 532 | NEUTROPHIL CYTOSOL FACTOR 1 | Homo sapiens |
| | CYTOSOL FACTOR 4 | | | | |
| 2krb | Eukaryotic translation | DEDVKDNWDD | 533 | Eukaryotic translation initiation | Homo sapiens |
| | initiation factor 3 | D | | factor 3 subunit J | |
| | subunit B | | | | |
| 2ks9 | Substance-P receptor | RPKPQQFFGLM | 534 | Substance P | Homo sapiens |
| 3gn8 | Glucocorticoid receptor | NALLRYLLDK | 535 | Nuclear receptor coactivator 2 | Homo sapiens |
| | 2 | D | | | |
| 3118 | Serine/threonine- | EPIAIIITDTE | 536 | AKAP79 peptide | Homo sapiens |
| | protein phosphatase 2B | | | | |
| | catalytic subunit alpha | | | | |
| | isoform | | | | |
| 4wjp | Small ubiquitin-related | EAEERIIVLXD | 537 | Daxx | Homo sapiens |
| | modifier 1 | | | | |
| 4yk6 | Adenomatous polyposis | YQGGGEEMAL | 538 | APC membrane recruitment protein 1 | Homo sapiens |
| | coli protein | P | | | |
| 5mf9 | RNA-binding protein 5 | GMRPPPPGIRG | 539 | Survival motor neuron protein | Homo sapiens |
| 2mwp | Tumor suppressor p53- | STSRHKXLMFK | 540 | Cellular tumor antigen p53 | Homo sapiens |
| | binding protein 1 | T | | | |
| 2rol | Epidermal growth | PPVPNPDYEPIR | 541 | 12-meric peptide from T-cell surface | Homo sapiens |
| | factor receptor kinase | | | glycoprotein CD3 epsilon chain | |
| | substrate 8-like protein | | | | |
| | 1 | | | | |
| 2seb | HLA CLASS II | AYMRADAAAG | 542 | PEPTIDE FROM COLLAGEN II | Homo sapiens |
| | HISTOCOMPATIBILI | GA | | | |
| | TY ANTIGEN | | | | |
| 3plz | FTZ-F1 related protein | KENALLRYLLD | 543 | Nuclear receptor coactivator 2 | Homo sapiens |
| | | K | | | |
| 4d07 | DYNEIN LIGHT | QPKDDKNTMT | 544 | MYOSIN VA VARIANT | Homo sapiens |
| | CHAIN 2, | DS | | | |
| | CYTOPLASMIC | | | | |
| 4dk7 | Oxysterols receptor | DHQLLRYLLD | 545 | Nuclear receptor coactivator 1 | Homo sapiens |
| | LXR-beta | KD | | | |
| 4naw | Ubiquitin-like protein | AADMEEYEES | 546 | Ubiquitin-like-conjugating enzyme ATG3 | Homo sapiens |
| | ATG12 | GL | | | |
| 4p6w | Glucocorticoid receptor | ANALLRYLLD | 547 | Nuclear receptor coactivator 2 | Homo sapiens |
| | | KD | | | |
| 4qjr | Steroidogenic factor 1 | PSLLKKLLLAP | 548 | Peroxisome proliferator-activated receptor | Homo sapiens |
| | | A | | gamma coactivator 1-alpha | |
| 4z0r | Zinc finger CW-type | ARTXQTARKS | 549 | Histone H3.1 | Homo sapiens |
| | PWWP domain protein | XX | | | |
| | 2 | | | | |
| 5buo | Amyloid beta A4 | FSLDDLQPWHS | 550 | Amyloid beta A4 protein | Homo sapiens |
| | protein | F | | | |
| 1aqd | HLA-DRI CLASS II | SDWRFLRGYH | 551 | HLA-A2 | Homo sapiens |
| | HISTOCOMPATIBILI | QYA | | | |
| | TY PROTEIN | | | | |
| 1cqg | THIOREDOXIN | PATLKICSWNV | 552 | REF-1 PEPTIDE | Homo sapiens |
| | | DG | | | |
| 100p | Splicing factor U2AF | PSKKRKRSRW | 553 | Splicing Factor SF1 | Homo sapiens |
| | 65 kDa subunit | NOD | | | |
| 3nmx | APC variant protein | GGGEQLAINEL | 554 | Rho guanine nucleotide exchange factor 4 | Homo sapiens |
| | | IS | | | |
| 3wxa | Programmed cell death | NPPPPGFIMHG | 555 | Protein transport protein Sec31A | Homo sapiens |
| | protein 6 | NX | | | |
| 4m7c | Telomeric repeat- | SRGLEVSHRLA | 556 | Peptide from Structure-specific endonuclease | Homo sapiens |
| | binding factor 2 | PW | | subunit SLX4 | |
| 5v3r | Programmed cell death | EDDDIKQLAA | 557 | Charged multivesicular body protein 4c | Homo sapiens |
| | 6-interacting protein | WAT | | | |
| 5xpt | Mitotic spindle | SGPWKPAKPAP | 558 | Chromosome alignment-maintaining | Homo sapiens |
| | assembly checkpoint | SV | | phosphoprotein 1 | |
| | protein MAD2B | | | | |
| 6bnh | Bromodomain- | KWTLERLKRK | 559 | Bifunctional arginine demethylase and lysyl- | Homo sapiens |
| | containing protein 4 | YRN | | hydroxylase JMJD6 | |
| 1mv0 | Myc box-dependent- | LLPTPPLSPSRR | 560 | Myc proto-oncogene protein | Homo sapiens |
| | interacting protein 1 | SG | | | |
| 2loz | Tensin-like Cl domain- | EDHKPGTFPKA | 561 | Rho GTPase-activating protein 7 | Homo sapiens |
| | containing phosphatase | LTN | | | |
| 3kv4 | PHD finger protein 8 | ARTXQTARXST | 562 | Histone H3-like | Homo sapiens |
| | | GGK | | | |
| 5dbr | Calmodulin | LNKYQGFIFDI | 563 | Sodium channel protein type 5 subunit alpha | Homo sapiens |
| | | VTK | | | |
| 5x8q | Nuclear receptor ROR- | NMGLEAIIRKA | 564 | Nuclear receptor corepressor 2 | Homo sapiens |
| | gamma | LMG | | | |
| 6b9h | Protein Hook homolog | EGVLANFFNSL | 565 | Cytoplasmic dynein 1 light intermediate | Homo sapiens |
| | 3 | LSK | | chain 1 | |
| 1klg | HLA CLASS II | GELIGILNAAK | 566 | Triosephosphate isomerase peptide | Homo sapiens |
| | HISTOCOMPATIBILI | VPAD | | | |
| | TY ANTIGEN, DR-1 | | | | |
| | BETA CHAIN | | | | |
| 21bm | Transcriptional | ARTKQTARXST | 567 | histone tail H3 K9me3 | Homo sapiens |
| | regulator ATRX | GGKA | | | |
| 2mg5 | Calmodulin | FKEVANAVKIS | 568 | target peptide | Homo sapiens |
| | | ASLM | | | |
| 2mwy | Protein Mdm4 | SQETFSDLWKL | 569 | Cellular tumor antigen p53 | Homo sapiens |
| | | LPEN | | | |
| 2r28 | Calmodulin | IRNKIRAIGKM | 570 | Serine/threonine-protein phosphatase 2B | Homo sapiens |
| | | ARVF | | catalytic subunit alpha isoform | |
| 2×04 | POLYADENYLATE- | WPPEFHPGVP | 571 | TRINUCLEOTIDE REPEAT-CONTAINING | Homo sapiens |
| | BINDING PROTEIN 1 | WKGLQ | | GENE 6C PROTEIN | |
| 3cfs | Histone-binding protein | QGITKPAIRRL | 572 | Histone H4 | Homo sapiens |
| | RBBP7 | ARRG | | | |
| 316f | HLA class II | APPAYEKLXAE | 573 | Melanoma antigen recognized by T-cells 1 | Homo sapiens |
| | histocompatibility | QSPP | | | |
| | antigen, DR alpha | | | | |
| | chain | | | | |
| 4rav | Single-chain Fv, VL | TLEKLMKAFES | 574 | Huntingtin | Homo sapiens |
| | | LKSF | | | |
| 6d94 | Peroxisome | GNTKNHPMLM | 575 | Mediator of RNA polymerase II transcription | Homo sapiens |
| | proliferator-activated | NLLKD | | subunit 1 | |
| | receptor gamma | | | | |
| 2axf | HLA class I | APQPAPENAY | 576 | 10-mer peptide from BZLFI trans-activator | Human |
| | histocompatibility | | | protein | gammaherpes |
| | antigen, B*3508 heavy | | | | virus 4 |
| | chain | | | | |
| 2bvp | HLA CLASS I | ISPRTLDAW | 577 | HIV-P24 | Human |
| | HISTOCOMPATIBILI | | | | immunodeficiency |
| | TY ANTIGEN, B-57 | | | | virus |
| | ALPHA CHAIN | | | | |
| 2f5b | PROTEIN | ELDKWAS | 578 | PROTEIN (GP41 EPITOPE) | Human |
| | (ANTIBODY 2F5 | | | | immunodeficiency |
| | (HEAVY CHAIN)) | | | | virus 1 |
| 1aln | HLA class I | VPLRPMTY | 579 | PEPTIDE VPLRPMTY | Human |
| | histocompatibility | | | | immunodeficiency |
| | | | | | virus 1 |
| | antigen, BW-53 | | | | |
| | B*5301 alpha chain | | | | |
| 1agb | B*0801 | GGRKKYKL | 580 | HIV-1 GAG PEPTIDE (GGRKKYKL (SEQ ID | Human |
| | | | | NO: 580) - 3R MUTATION) | immunodeficiency |
| | | | | | virus 1 |
| 1fgl | CYCLOPHILIN A | VHAGPIAPGQX | 581 | HIV-1 GAG PROTEIN | Human |
| | | R | | | immunodeficiency |
| | | | | | virus 1 |
| 2r7g | Retinoblastoma- | PPTLHELYD | 582 | Early ElA 32 kDa protein | Human |
| | associated protein | | | | mastadenovirus |
| | | | | | C |
| 1gux | RETINOBLASTOMA | DLYCYEQLN | 583 | ONCOPROTEIN | Human |
| | PROTEIN | | | | papillomavirus |
| 4045 | WD repeat-containing | ARTAR | 584 | Nonstructural protein 1 | Influenza A |
| | protein 5 | | | | virus |
| 4042 | Chromodomain- | RTARSXVX | 585 | Nonstructural protein 1 | Influenza A |
| | helicase-DNA-binding | | | | virus |
| | protein 1 | | | | |
| 1pyw | HLA class II | FVKQNAXAL | 586 | 9-residue influenza virus hemagglutinin | Influenza A |
| | histocompatibility | | | related peptide FVKQNA(MAA)*AL (SEQ ID NO: | virus |
| | antigen, DR alpha | | | 586) | |
| | chain | | | *MAA: N-methyl-L-alanine | |
| 1aot | FYN PROTEIN- | EPQXEEIPIYL | 587 | PHOSPHOTYROSYL PEPTIDE | Mesocricetus |
| | TYROSINE KINASE | | | | auratus |
| | | | | | polyomavirus 1 |
| 4i51 | Serine/threonine- | XLXRXXX | 588 | Microcystin-LR (MCLR) bound form | Microcystis |
| | protein phosphatase 2A | | | | aeruginosa |
| | catalytic subunit alpha | | | | |
| | isoform, PP2A-alpha | | | | |
| 3w8j | Protein disulfide- | EVCPAG | 589 | C-terminal peptide from Peroxiredoxin-4 | Mus musculus |
| | isomerase A6 | | | | |
| 5jiu | Ran-binding protein 9 | ESSDSQ | 590 | Probable ATP-dependent RNA helicase DDX4 | Mus musculus |
| 2zjd | Microtubule-associated | DDWTHLS | 591 | undecameric peptide from Sequestosome-1 | Mus musculus |
| | proteins 1A/1B light | | | | |
| | chain 3B precursor | | | | |
| 112i | ESTROGEN | HKILHRLLQD | 592 | GLUCOCORTICOID RECEPTOR- | Mus musculus |
| | RECEPTOR | | | INTERACTING PROTEIN 1 | |
| 4dma | Estrogen receptor | HKILHRLLQEG | 593 | Nuclear receptor coactivator 1 | Mus musculus |
| 41x2 | Unconventional | KKRLLSFRDVD | 594 | Melanophilin | Mus musculus |
| | myosin-Va | F | | | |
| 4gxb | Sorting nexin-17 | GTYGVFTNAA | 595 | P-selectin | Mus musculus |
| | | YDPTP | | | |
| 5xxk | E3 ubiquitin-protein | XSFXEYXALLX | 596 | Hydrocarbon stapled peptide THC*- SER-PHE- | Phage display |
| | ligase Mdm2 | X | | OEH**- GLU-TYR-6CW***- ALA-LEU-LEU- | vector pTDisp |
| | | | | MK8****- NH2 | |
| | | | | (SEQ ID NO: 596) | |
| | | | | *THC: N-methylcarbonylthreonine | |
| | | | | **0EH: (2R)-2-amino-2-methylnonanoic acid | |
| | | | | ***6CW: 6-CHLORO-L-TRYPTOPHAN | |
| | | | | ****MK8: 2-methyl-L-norleucine | |
| 1thr | ALPHA-THROMBIN | SDFEEFSLDDIE | 597 | HIRULLIN | Poecilobdella |
| | (LARGE SUBUNIT) | Q | | | manillensis |
| 1bd2 | HLA-A 0201 | LLFGYPVYV | 598 | TAX PEPTIDE | Primate T- |
| | | | | | lymphotropic |
| | | | | | virus 1 |
| 4e5x | HLA class I | LLFGYPVYV | 599 | Protein Tax-1 | Primate T- |
| | histocompatibility | | | | lymphotropic |
| | antigen, A-2 alpha | | | | virus 1 |
| | chain | | | | |
| 2kqf | Microtubule-associated | SWESHKSGGET | 600 | C-terminal motif from Glycoprotein | Rabies |
| | serine/threonine-protein | RL | | | lyssavirus |
| | kinase 2 | | | | |
| 1yvh | CBL E3 ubiquitin | RAVENQXSF | 601 | 13-mer fragment of SH2 and PH domain- | Rattus |
| | protein ligase | | | containing adapter protein APS | norvegicus |
| 2iv9 | AP-2 COMPLEX | SFGDGFADF | 602 | EPIDERMAL GROWTH FACTOR | Rattus |
| | SUBUNIT BETA-2 | | | RECEPTOR SUBSTRATE 15 ISOFORM B | norvegicus |
| 6mue | Calmodulin-1 | EVCAIKIQRAY | 603 | Sodium channel protein type 4 subunit alpha | Rattus |
| | | RRHL | | | norvegicus |
| 4p9v | Growth factor receptor- | XXXNX | 604 | PHQ-PTR-02K-ASN-NH2 | synthetic |
| | bound protein 2 | | | | construct |
| 5ttw | Polycomb protein EED | PSXFX | 605 | UNC4859 | synthetic |
| | | | | | construct |
| 6ckz | Caspase-3 subunit p12 | XDXFX | 606 | ACE-1MH-ASP-B3L-PHE-1U8 | synthetic |
| | | | | | construct |
| 4q13 | Estrogen receptor | ILHRLL | 607 | Glucocorticoid receptor-interacting | synthetic |
| | | | | protein 1 NR box II peptide | construct |
| 5jmo | Furin | XRVKXX | 608 | CMK-inhibitor | synthetic |
| | | | | | construct |
| 4wyu | Protein scribble | SWFQTDL | 609 | peptide SER-TRP-PHE-GLN-THR-ASP-LEU | synthetic |
| | homolog | | | (SEQ ID NO: 609) | construct |
| 4xt9 | Nuclear receptor ROR- | KILHRLLQ | 610 | LYS-ILE-LEU-HIS-ARG-LEU-LEU-GLN (SEQ | synthetic |
| | gamma | | | ID NO: 610) | construct |
| 5bwn | NAD-dependent | TARXSTGGW | 611 | myristoyl H3K9 peptide | synthetic |
| | protein deacetylase | | | | construct |
| | sirtuin-3, mitochondrial | | | | |
| 5ulq | Growth factor receptor- | KFEGYDNEX | 612 | LYS-PHE-GLU-GLY-TYR-ASP-ASN-GLU- | synthetic |
| | bound protein 7 | | | CST* (SEQ ID NO: 612) | construct |
| | | | | *CST: 48V ( {[(2R)-2,3-diamino-3- | |
| | | | | oxopropyl\|sulfanyl }acetic acid ) | |
| 4x1q | Urokinase-type | CPAYSRYLDC | 613 | mupain-1 | synthetic |
| | plasminogen activator | | | | construct |
| 213r | E3 ubiquitin-protein | ARTKQTARXST | 614 | Histone H3 | synthetic |
| | ligase UHRF1 | | | | construct |
| 5f8z | Plasma kallikrein | CPARFAALFCX* | 615 | CYS-PRO-ALA-ARG-PHE-ALA-ALA-LEU- | synthetic |
| | LIGHT CHAIN | *X: intrachain | | PHE-CYS (SEQ ID NO: 615) | construct |
| | | disulfide bond | | | |
| 3ewt | Calmodulin | SKYITTIAGVM | 616 | Tumor necrosis factor receptor superfamily | synthetic |
| | | TLS | | member 6 | construct |
| 5vb9 | Interleukin-17A | CWVLEYDMFG | 617 | Peptide inhibitor | synthetic |
| | | ALHCR | | | construct |
| 1bck | PEPTIDYL-PROLYL | XXXXXTXXVX | 618 | CYCLOSPORIN C | Tolypocladium |
| | CIS-TRANS | A | | | inflatum |
| | ISOMERASE A | | | | |
| 1ikf | IGG1-KAPPA R45-45- | XXXXXXXXVX | 619 | CYCLOSPORIN A | Tolypocladium |
| | 11 FAB (HEAVY | A | | | inflatum |
| | CHAIN) | | | | |
| 2h9m | WD-repeat protein 5 | ARTKQ | 620 | H3 histone | Xenopus |
| | | | | | tropicalis |

“X” indicates any naturally occurring amino acid unless otherwise indicated.

The following table provides exemplary sequences for use in a binding unit in a binding domain of an engineered scaffold protein as described herein.

TABLE 4
Exemplary Binding unit Sequences

| Exemplary Amino Acid Sequence | Sequence ID NO: |
|---|---|
| MIKEEHVIIQAEFYLNPDQSGEFMFDFDGDEIFHVDMAKKETVWRLEEFGRFASFEAQGALANIAVDKANLEIMTKRSNY | 621 |
| TPITNV | |
| QSEAGSHTVQRMYGCDVGSDWRFLRGYHQYAY | 622 |
| MIKEEHVIIQAEFYLNPDQSGEFMFDFDGDEIFHVDMAKKETVWRLEEFGRFASFEAQGALANIAVDKANLEIMTKRSNY | 623 |
| TPITNVPPEVTVLTNSPVELREPNVLICFIDKFTPPVVNVTWLRNGKPVTTGVSETVFLPREDHLFRKFHYLPFLPSTED | |
| VYDCRVEHWGLDEPLLKHWEFDA | |
| GSHSMRYFFTSVSRPGRGEPRFIAVGYVDDTQFVRFDSDAASQRMEP | 624 |
| ADMAAQTTKHKWEAAHVAEQLRAYLEGTCVEWLRRYLENGKETLQRTDAPKTHMTHHAVSDHEATLRCWALSFYPAE | 625 |
| ITLTWQRDGEDQTQDTELVETRPAGDGTFQKWAAVVVPSGQEQRYTCHVQHEGLPKPLTLRWE | |
| QSEAGSHTVQRMYGCDVGSDWRFLRGYHQYAYDGKDYIALKEDLRSWTAADMAAQTTKHKWEAAHVAEQLRAYLEG | 626 |
| TCVEWLRRYLENGKETLQRTDAPKTHMTHHAVSDHEATLRCWALSFYPAEITLTWQRDGEDQTQDTELVETRPAGDGTF | |
| QKWAAVVVPSGQEQRYTCHVQHEGLPKPLTLRWE | |
| MGSHSMRYFFTSVSRPGRGEPRFIAVGYVDDTQFVRFDSDAASQRMEPRAPWIEQEGPEYWDGETRKVKAHSQTHRVDL | 627 |
| GTLRGYYNQSEAGSH | |

The following table provides exemplary sequences for use in a hinge unit in a binding domain of an engineered scaffold protein as described herein.

TABLE 5
Exemplary Hinge unit Sequences

| Exemplary Amino Acid Sequence | SEQ ID NO: |
|---|---|
| SDVGEYRAVTELGRPDAEYWNSQKDLLEQRRAAVDTYCRHNYGVGESFTVQRRVE | 628 |
| GDTRPRFLWQLKFECHFFNGTERVRLLERCIYNQEESVRFDSDVGEYRAVTELGRPDAEYWNSQKDLLEQRRAA | 629 |
| VDTYCRHNYGVGESFTVQRRVE | |
| MGDTRPRFLWQLKFECHFFNGTERVRLLERCIYNQEESVRFDSDVGEYRAVTELGRPDAEYWNSQKDLLEQRRA | 630 |
| AVDTYCRHNYGVGESFTVQRRVE | |
| MGDTRPRFLWQLKFECHFFNGTERVRLLERCIYNQEESVRFDSDVGEYRAVTELGRPDAEYWNSQKDLLEQRRA | 631 |
| AVDTYCRHNYGVGESFTVQRRVE | |
| MGDTRPRFLWQLKFECHFFNGTERVRLLERCIYNQEESVRFDSDVGEYRAVTELGRPDAEYWNSQKDLLEQRRA | 632 |
| AVDTYCRHNYGVGESFTVQRRVE | |
| GDTRPRFLWQLKFECHFFNGTERVRLLERCIYNQEESVRFDSDVGEYRAVTELGRPDAEYWNSQKDLLEQRRAA | 633 |
| VDTYCRHNYGVGESFTVQRRVEPKVTVYPSKTQPLQHHNLLVCSVSGFYPGSIEVRWFRNGQEEKAGVVSTGLI | |
| QNGDWTFQTLVMLETVPRSGEVYTCQVEHPSVTSPLTVEWRA | |
| GDTRPRFLWQLKFECHFFNGTERVRLLERCIYNQEESVRFDSDVGEYRA | 634 |
| GDTRPRFLWQLKFECHFFNGTERVRLLERCIYNQEESVRFDSDVGEYRAVTELGRPDAEYWNSQKDLLEQRRAA | 635 |
| VDTYCRHNYGVGESFTVQRRVEPKVTVYPSKTQPLQHHNLLVCSVSGFYPGSIEVRWFRNGQEEKAGVVSTGLI | |
| QNGDWTFQTLVMLETVPRSGEVYTCQVEHPSVTSPLTVEWRA | |

The following table provides exemplary sequences comprised in a disordered region as described herein.

TABLE 6
Exemplary Engineered Scaffold Sequences

| SEQ | | |
|---|---|---|
| ID | Scaffold | |
| NO: | Name | Amino Acid Sequence |
| 636 | Chimera 1 | MIKEEHVIIQAEFYLNPDQSGEFMFDFDGDEIFHVDMAKKETVWRLEEFGRFASFEAQGALANIAVDKANL |
| | | EIMTKRSNYTPITNVQSEAGSHTVQRMYGCDVGSDWRFLRGYHQYAYSDVGEYRAVTELGRPDAEYWNS |
| | | QKDLLEQRRAAVDTYCRHNYGVGESFTVQRRVE |
| 637 | Chimera 2-1 | MIKEEHVIIQAEFYLNPDQSGEFMFDFDGDEIFHVDMAKKETVWRLEEFGRFASFEAQGALANIAVDKANL |
| | | EIMTKRSNYTPITNVGGGGSGGGGSGGGGSGDTRPRFLWQLKFECHFFNGTERVRLLERCIYNQEESVRFD |
| | | SDVGEYRAVTELGRPDAEYWNSQKDLLEQRRAAVDTYCRHNYGVGESFTVQRRVE |
| 638 | Chimera 2-2 | MIKEEHVIIQAEFYLNPDQSGEFMFDFDGDEIFHVDMAKKETVWRLEEFGRFASFEAQGALANIAVDKANL |
| | | EIMTKRSNYTPITNVGGGGGGGGGDTRPRFLWQLKFECHFFNGTERVRLLERCIYNQEESVRFDSDVGEYR |
| | | AVTELGRPDAEYWNSQKDLLEQRRAAVDTYCRHNYGVGESFTVQRRVE |
| 639 | Chimera 2-3 | MIKEEHVIIQAEFYLNPDQSGEFMFDFDGDEIFHVDMAKKETVWRLEEFGRFASFEAQGALANIAVDKANL |
| | | EIMTKRSNYTPITNVGGGGGGGDTRPRFLWQLKFECHFFNGTERVRLLERCIYNQEESVRFDSDVGEYRAV |
| | | TELGRPDAEYWNSQKDLLEQRRAAVDTYCRHNYGVGESFTVQRRVE |
| 640 | Chimera 2-4 | MIKEEHVIIQAEFYLNPDQSGEFMFDFDGDEIFHVDMAKKETVWRLEEFGRFASFEAQGALANIAVDKANL |
| | | EIMTKRSNYTPITNVGGGGSGDTRPRFLWQLKFECHFFNGTERVRLLERCIYNQEESVRFDSDVGEYRAVTE |
| | | LGRPDAEYWNSQKDLLEQRRAAVDTYCRHNYGVGESFTVQRRVE |
| 641 | Chimera 2-5 | MIKEEHVIIQAEFYLNPDQSGEFMFDFDGDEIFHVDMAKKETVWRLEEFGRFASFEAQGALANIAVDKANL |
| | | EIMTKRSNYTPITNVLEGDTRPRFLWQLKFECHFFNGTERVRLLERCIYNQEESVRFDSDVGEYRAVTELGR |
| | | PDAEYWNSQKDLLEQRRAAVDTYCRHNYGVGESFTVQRRVE |
| 642 | Chimera 2-6 | MIKEEHVIIQAEFYLNPDQSGEFMFDFDGDEIFHVDMAKKETVWRLEEFGRFASFEAQGALANIAVDKANL |
| | | EIMTKRSNYTPITNVGGGGSGGGGSGDTRPRFLWQLKFECHFFNGTERVRLLERCIYNQEESVRFDSDVGE |
| | | YRAVTELGRPDAEYWNSQKDLLEQRRAAVDTYCRHNYGVGESFTVQRRVE |
| 643 | Chimera 2-7 | MGDTRPRFLWQLKFECHFFNGTERVRLLERCIYNQEESVRFDSDVGEYRAVTELGRPDAEYWNSQKDLLE |
| | | QRRAAVDTYCRHNYGVGESFTVQRRVELEMIKEEHVIIQAEFYLNPDQSGEFMFDFDGDEIFHVDMAKKET |
| | | VWRLEEFGRFASFEAQGALANIAVDKANLEIMTKRSNYTPITNV |
| 644 | Chimera 2-8 | MGDTRPRFLWQLKFECHFFNGTERVRLLERCIYNQEESVRFDSDVGEYRAVTELGRPDAEYWNSQKDLLE |
| | | QRRAAVDTYCRHNYGVGESFTVQRRVEQSEAGSHMIKEEHVIIQAEFYLNPDQSGEFMFDFDGDEIFHVDM |
| | | AKKETVWRLEEFGRFASFEAQGALANIAVDKANLEIMTKRSNYTPITNV |
| 645 | Chimera 2-9 | MGDTRPRFLWQLKFECHFFNGTERVRLLERCIYNQEESVRFDSDVGEYRAVTELGRPDAEYWNSQKDLLE |
| | | QRRAAVDTYCRHNYGVGESFTVQRRVEQSEAGSHMIKEEHVIIQAEFYLNPDQSGEFMFDFDGDEIFHVDM |
| | | AKKETVWRLEEFGRFASFEAQGALANIAVDKANLEIMTKRSNYTPITNVPPEVTVLTNSPVELREPNVLICFI |
| | | DKFTPPVVNVTWLRNGKPVTTGVSETVFLPREDHLFRKFHYLPFLPSTEDVYDCRVEHWGLDEPLLKHWE |
| | | FDA |
| 646 | Chimera 2- | MIKEEHVIIQAEFYLNPDQSGEFMFDFDGDEIFHVDMAKKETVWRLEEFGRFASFEAQGALANIAVDKANL |
| | 10 | EIMTKRSNYTPITNVLEGDTRPRFLWQLKFECHFFNGTERVRLLERCIYNQEESVRFDSDVGEYRAVTELGR |
| | | PDAEYWNSQKDLLEQRRAAVDTYCRHNYGVGESFTVQRRVEPKVTVYPSKTQPLQHHNLLVCSVSGFYP |
| | | GSIEVRWFRNGQEEKAGVVSTGLIQNGDWTFQTLVMLETVPRSGEVYTCQVEHPSVTSPLTVEWRA |
| 647 | Chimera 2-5- | MIKEEHVIIQAEFYLNPDQSGEFMFDFDGDEIFHVDMAKKETVWRLEEFGRFASFEAQGALANIAVDKANL |
| | Fusion | EIMTKRSNYTPITNVLEGDTRPRFLWQLKFECHFFNGTERVRLLERCIYNQEESVRFDSDVGEYRAVTELGR |
| | | PDAEYWNSQKDLLEQRRAAVDTYCRHNYGVGESFTVQRRVESSGMKAIFVLNAQHDEAVDANSLAEAKV |
| | | LANRELDKYGVSDYYKNLINNAKTVEGVKALIDEILAALP |
| 648 | Chimera 3 | GSHSMRYFFTSVSRPGRGEPRFIAVGYVDDTQFVRFDSDAASQRMEPRLEEFGRFASFEAQGALANIAVDK |
| | | ANLEIMTKRSNYTPITNVLEGDTRPRFLWQLKFECHFFNGTERVRLLERCIYNQEESVRFDSDVGEYRAAD |
| | | MAAQTTKHKWEAAHVAEQLRAYLEGTCVEWLRRYLENGKETLQRTDAPKTHMTHHAVSDHEATLRCW |
| | | ALSFYPAEITLTWQRDGEDQTQDTELVETRPAGDGTFQKWAAVVVPSGQEQRYTCHVQHEGLPKPLTLRW |
| | | E |
| 649 | Chimera 4-1 | MGDTRPRFLWQLKFECHFFNGTERVRLLERCIYNQEESVRFDSDVGEYRAVTELGRPDAEYWNSQKDLLE |
| | | QRRAAVDTYCRHNYGVGESFTVQRRVEQSEAGSHTVQRMYGCDVGSDWRFLRGYHQYAYDGKDYIALK |
| | | EDLRSWTAADMAAQTTKHKWEAAHVAEQLRAYLEGTCVEWLRRYLENGKETLQRTDAPKTHMTHHAV |
| | | SDHEATLRCWALSFYPAEITLTWQRDGEDQTQDTELVETRPAGDGTFQKWAAVVVPSGQEQRYTCHVQH |
| | | EGLPKPLTLRWE |
| 650 | Chimera 4-2 | MGSHSMRYFFTSVSRPGRGEPRFIAVGYVDDTQFVRFDSDAASQRMEPRAPWIEQEGPEYWDGETRKVKA |
| | | HSQTHRVDLGTLRGYYNQSEAGSHGDTRPRFLWQLKFECHFFNGTERVRLLERCIYNQEESVRFDSDVGEY |
| | | RAVTELGRPDAEYWNSQKDLLEQRRAAVDTYCRHNYGVGESFTVQRRVEPKVTVYPSKTQPLQHHNLLV |
| | | CSVSGFYPGSIEVRWFRNGQEEKAGVVSTGLIQNGDWTFQTLVMLETVPRSGEVYTCQVEHPSVTSPLTVE |
| | | WRA |
| 651 | S001 | MIKEEHVIIQAEFYLNPDQSGEFMFDFDGDEIFHVDMAKKETVWRLEEFGRFASFEAQGALANIAVDKANL |
| | | EIMTKRSNYTPLEMGDTRPRFLWQLKFECHFFNGTERVRLLERCIYNQEESVRFDSDVGEYRAVTELGRPD |
| | | AEYWNSQKDLLEQRRAAVDTYCRHNYGVGESFTVQGGSHHHHHHLE |
| 652 | S002 | MIKEEHVIIQAEFYLNPDQSGEFMFDFDGDEIFHVDMAKKETVWRLEEFGRFASFEAQGALANIAVDKANL |
| | | EIMTKRSNYTPGGSMGDTRPRFLWQLKFECHFFNGTERVRLLERCIYNQEESVRFDSDVGEYRAVTELGRP |
| | | DAEYWNSQKDLLEQRRAAVDTYCRHNYGVGESFTVQGGSHHHHHHLE |
| 653 | S003 | MIKEEHVIIQAEFYLNPDQSGEFMFDFDGDEIFHVDMAKKETVWRLEEFGRFASFEAQGALANIAVDKANL |
| | | EIMTKRSNYTPSSGMGDTRPRFLWQLKFECHFFNGTERVRLLERCIYNQEESVRFDSDVGEYRAVTELGRP |
| | | DAEYWNSQKDLLEQRRAAVDTYCRHNYGVGESFTVQGGSHHHHHHLE |
| 654 | S004 | MIKEEHVIIQAEFYLNPDQSGEFMFDFDGDEIFHVDMAKKETVWRLEEFGRFASFEAQGALANIAVDKANL |
| | | EIMTKRSNYTPGGGGSMGDTRPRFLWQLKFECHFFNGTERVRLLERCIYNQEESVRFDSDVGEYRAVTELG |
| | | RPDAEYWNSQKDLLEQRRAAVDTYCRHNYGVGESFTVQGGSHHHHHHLE |
| 655 | S005 | MIKEEHVIIQAEFYLNPDQSGEFMFDFDGDEIFHVDMAKKETVWRLEEFGRFASFEAQGALANIAVDKANL |
| | | EIMTKRSNYTPMGDTRPRFLWQLKFECHFFNGTERVRLLERCIYNQEESVRFDSDVGEYRAVTELGRPDAE |
| | | YWNSQKDLLEQRRAAVDTYCRHNYGVGESFTVQGGSHHHHHHLE |
| 656 | S006 | MIKEEHVIIQAEFYLNPDQSGEFMFDFDGDEIFHVDMAKKETVWRLEEFGRFASFEAQGALANIAVDKANL |
| | | EIMTKRSNYTPPVSKMRMATPLLMQALPMGDTRPRFLWQLKFECHFFNGTERVRLLERCIYNQEESVRFDS |
| | | DVGEYRAVTELGRPDAEYWNSQKDLLEQRRAAVDTYCRHNYGVGESFTVQGGSHHHHHHLE |
| 657 | S007 | MIKEEHVIIQAEFYLNPDQSGEFMFDFDGDEIFHVDMAKKETVWRLEEFGRFASFEAQGALANIAVDKANL |
| | | EIMTKRSNYTPMGDTRPRFLWQLKFECHFFNGTERVRLLERCIYNQEESVRFDSDVGEYRAVTELGRPDAE |
| | | YWNSQKDLLEQRRAAVDTYCRHNYGVGESFTVQRRVEPQRTPKIQVYSRHPAENGKSNFLNCYVSGFHPS |
| | | DIEVDLLKNGERIEKVEHSDLSFSKDWSFYLLYYTEFTPTEKDEYACRVNHVTLSQPKIVKWDRDMGGSHH |
| | | HHHHLE |
| 658 | S008 | MIKEEHVIIQAEFYLNPDQSGEFMFDFDGDEIFHVDMAKKETVWRLEEFGRFASFEAQGALANIAVDKANL |
| | | EIMTKRSNYTPLEMGDTRPRFLWQLKFECHFFNGTERVRLLERCIYNQEESVRFDSDVGEYRAVTELGRPD |
| | | AEYWNSQKDLLEQRRAAVDTYCRHNYGVGESFTVQGGSMKAIFVLNAQHDEAVDANSLAEAKVLANRE |
| | | LDKYGVSDYYKNLINNAKTVEGVKALIDEILAALPGGSHHHHHHLE |
| 659 | S009 | MIKEEHVIIQAEFYLNPDQSGEFMFDFDGDEIFHVDMAKKETVWRLEEFGRFASFEAQGALANIAVDKANL |
| | | EIMTKRSNYTPLEMGDTRPRFLWQLKFECHFFNGTERVRLLERCIYNQEESVRFDSDVGEYRAVTELGRPD |
| | | AEYWNSQKDLLEQRRAAVDTYCRHNYGVGESFTVQGGSKTKNVVCNEELTELAEAKVLANRELDKYGVS |
| | | DYHKNLINNAKTVEGVKDLQAQVVESAGGSHHHHHHLE |
| 660 | S010 | MIKEEHVIIQAEFYLNPDQSGEFMFDFDGDEIFHVDMAKKETVWRLEEFGRFASFEAQGALANIAVDKANL |
| | | EIMTKRSNYTPLEMGDTRPRFLWQLKFECHFFNGTERVRLLERCIYNQEESVRFDSDVGEYRAVTELGRPD |
| | | AEYWNSQKDLLEQRRAAVDTYCRHNYGVGESFTVQGGSKTKNVVCNEELTALAKAKVLANRELDKYGV |
| | | SDYYKKLVNKAKAAETVATLQAQVVESAKKARGGSHHHHHHLE |
| 661 | S011 | MIKEEHVIIQAEFYLNPDQSGEFMFDFDGDEIFHVDMAKKETVWRLEEFGRFASFEAQGALANIAVDKANL |
| | | EIMTKRSNYTPLEMGDTRPRFLWQLKFECHFFNGTERVRLLERCIYNQEESVRFDSDVGEYRAVTELGRPD |
| | | AEYWNSQKDLLEQRRAAVDTYCRHNYGVGESFTVQGGSMKAIFVLNAQHDEAVDANSALAKAKVLANR |
| | | ELDKYGVSDYYKKLVNNAKTVEGVKDLQAQVVESAKKARGGSHHHHHHLE |
| 662 | S012 | MIKEEHVIIQAEFYLNPDQSGEFMFDFDGDEIFHVDMAKKETVWRLEEFGRFASFEAQGALANIAVDKANL |
| | | EIMTKRSNYTPMGDTRPRFLWQLKFECHFFNGTERVRLLERCIYNQEESVRFDSDVGEYRAVTELGRPDAE |
| | | YWNSQKDLLEQRRAAVDTYCRHNYGVGESFTVQRRVEPQRTPKIQVYSRHPAENGKSNFLNCYVSGFHPS |
| | | DIEVDLLKNGERIEKVEHSDLSFSKDWSFYLLYYTEFTPTEKDEYACRVNHVTLSQPKIVKWDRDMGGSM |
| | | KAIFVLNAQHDEAVDANSLAEAKVLANRELDKYGVSDYYKNLINNAKTVEGVKALIDEILAALPGGSENL |
| | | YFQGGSHHHHHHLE |
| 663 | S013 | MIKEEHVIIQAEFYLNPDQSGEFMFDFDGDEIFHVDMAKKETVWRLEEFGRFASFEAQGALANIAVDKANL |
| | | EIMTKRSNYTPMGDTRPRFLWQLKFECHFFNGTERVRLLERCIYNQEESVRFDSDVGEYRAVTELGRPDAE |
| | | YWNSQKDLLEQRRAAVDTYCRHNYGVGESFTVQRRVEPQRTPKIQVYSRHPAENGKSNFLNCYVSGFHPS |
| | | DIEVDLLKNGERIEKVEHSDLSFSKDWSFYLLYYTEFTPTEKDEYACRVNHVTLSQPKIVKWDRDMGGSM |
| | | KAIFVLNAQHDEAVDANSALAKAKVLANRELDKYGVSDYYKKLVNNAKTVEGVKDLQAQVVESAKKAR |
| | | GGSHHHHHHLE |
| 664 | S014 | MIKEEHVIIQAEFYLNPDQSGEFMFDFDGDEIFHVDMAKKETVWRLEEFGRFASFEAQGALANIAVDKANL |
| | | EIMTKRSNYTPMGDTRPRFLWQLKFECHFFNGTERVRLLERCIYNQEESVRFDSDVGEYRAVTELGRPDAE |
| | | YWNSQKDLLEQRRAAVDTYCRHNYGVGESFTVQRRVEPMKAIFVLNAQHDEAVDANSLAEAKVLANRE |
| | | LDKYGVSDYYKNLINNAKTVEGVKALIDEILAALPGGSQRTPKIQVYSRHPAENGKSNFLNCYVSGFHPSDI |
| | | EVDLLKNGERIEKVEHSDLSFSKDWSFYLLYYTEFTPTEKDEYACRVNHVTLSQPKIVKWDRDMGGSHHH |
| | | HHHLE |
| 665 | S015 | MIKEEHVIIQAEFYLNPDQSGEFMFDFDGDEIFHVDMAKKETVWRLEEFGRFASFEAQGALANIAVDKANL |
| | | EIMTKRSNYTPMGDTRPRFLWQLKFECHFFNGTERVRLLERCIYNQEESVRFDSDVGEYRAVTELGRPDAE |
| | | YWNSQKDLLEQRRAAVDTYCRHNYGVGESFTVQRRVEPMKAIFVLNAQHDEAVDANSALAKAKVLANR |
| | | ELDKYGVSDYYKKLVNNAKTVEGVKDLQAQVVESAKKARGGSQRTPKIQVYSRHPAENGKSNFLNCYVS |
| | | GFHPSDIEVDLLKNGERIEKVEHSDLSFSKDWSFYLLYYTEFTPTEKDEYACRVNHVTLSQPKIVKWDRDM |
| | | GGSHHHHHHLE |
| 666 | S016 | MKAIFVLNAQHDEAVDANSLAEAKVLANRELDKYGVSDYYKNLINNAKTVEGVKALIDEILAALPGGSMI |
| | | KEEHVIIQAEFYLNPDQSGEFMFDFDGDEIFHVDMAKKETVWRLEEFGRFASFEAQGALANIAVDKANLEI |
| | | MTKRSNYTPMGDTRPRFLWQLKFECHFFNGTERVRLLERCIYNQEESVRFDSDVGEYRAVTELGRPDAEY |
| | | WNSQKDLLEQRRAAVDTYCRHNYGVGESFTVQRRVEPQRTPKIQVYSRHPAENGKSNFLNCYVSGFHPSD |
| | | IEVDLLKNGERIEKVEHSDLSFSKDWSFYLLYYTEFTPTEKDEYACRVNHVTLSQPKIVKWDRDMGGSHHH |
| | | HHHLE |
| 667 | S017 | MKAIFVLNAQHDEAVDANSALAKAKVLANRELDKYGVSDYYKKLVNNAKTVEGVKDLQAQVVESAKK |
| | | ARGGSMIKEEHVIIQAEFYLNPDQSGEFMFDFDGDEIFHVDMAKKETVWRLEEFGRFASFEAQGALANIAV |
| | | DKANLEIMTKRSNYTPMGDTRPRFLWQLKFECHFFNGTERVRLLERCIYNQEESVRFDSDVGEYRAVTELG |
| | | RPDAEYWNSQKDLLEQRRAAVDTYCRHNYGVGESFTVQRRVEPQRTPKIQVYSRHPAENGKSNFLNCYVS |
| | | GFHPSDIEVDLLKNGERIEKVEHSDLSFSKDWSFYLLYYTEFTPTEKDEYACRVNHVTLSQPKIVKWDRDM |
| | | GGSHHHHHHLE |
| 668 | S018 | MIKEEHVIIQAEFYLNPDQSGEFMFDFDGDEIFHVDMAKKETVWRLEEFGRFASFEAQGALANIAVDKANL |
| | | EIMTKRSNYTPMGDTRPRFLWQLKFECHFFNGTERVRLLERCIYNQEESVRFDSDVGEYRAVTELGRPDAE |
| | | YWNSQKDLLEQRRAAVDTYCRHNYGVGESFTVQRRVEPKVTVYPSKTQPLQHHNLLVCSVSGFYPGSIEV |
| | | RWFRNGQEEKAGVVSTGLIQNGDWTFQTLVMLETVPRSGEVYTCQVEHPSVTSPLTVEWRARSESAQSKG |
| | | GSMKAIFVLNAQHDEAVDANSLAEAKVLANRELDKYGVSDYYKNLINNAKTVEGVKALIDEILAALPGGS |
| | | HHHHHHLE |
| 669 | S019 | MIKEEHVIIQAEFYLNPDQSGEFMFDFDGDEIFHVDMAKKETVWRLEEFGRFASFEAQGALANIAVDKANL |
| | | EIMTKRSNYTPMGDTRPRFLWQLKFECHFFNGTERVRLLERCIYNQEESVRFDSDVGEYRAVTELGRPDAE |
| | | YWNSQKDLLEQRRAAVDTYCRHNYGVGESFTVQRRVEPKVTVYPSKTQPLQHHNLLVCSVSGFYPGSIEV |
| | | RWFRNGQEEKAGVVSTGLIQNGDWTFQTLVMLETVPRSGEVYTCQVEHPSVTSPLTVEWRARSESAQSKG |
| | | GSMKAIFVLNAQHDEAVDANSALAKAKVLANRELDKYGVSDYYKKLVNNAKTVEGVKDLQAQVVESAK |
| | | KARGGSHHHHHHLE |
| 670 | S020 | MIKEEHVIIQAEFYLNPDQSGEFMFDFDGDEIFHVDMAKKETVWRLEEFGRFASFEAQGALANIAVDKANL |
| | | EIMTKRSNYTPLEMGDTRPRFLWQLKFECHFFNGTERVRLLERCIYNQEESVRFDSDVGEYRAVTELGRPD |
| | | AEYWNSQKDLLEQRRAAVDTYCRHNYGVGESFTVQGGSMKAIFVLNAQHDEAVDANSALAKAKVLANR |
| | | ELDKYGVSDYYKKLVNNAKTVEGVKDLQAQVVESAKKARGGSQRTPKIQVYSRHPAENGKSNFLNCYVS |
| | | GFHPSDIEVDLLKNGERIEKVEHSDLSFSKDWSFYLLYYTEFTPTEKDEYACRVNHVTLSQPKIVKWDRDM |
| | | GGSHHHHHHLE |
| 671 | S021 | MIKEEHVIIQAEFYLNPDQSGEFMFDFDGDEIFHVDMAKKETVWRLEEFGRFASFEAQGALANIAVDKANL |
| | | EIMTKRSNYTPLEMGDTRPRFLWQLKFECHFFNGTERVRLLERCIYNQEESVRFDSDVGEYRAVTELGRPD |
| | | AEYWNSQKDLLEQRRAAVDTYCRHNYGVGESFTVQGGSQRTPKIQVYSRHPAENGKSNFLNCYVSGFHPS |
| | | DIEVDLLKNGERIEKVEHSDLSFSKDWSFYLLYYTEFTPTEKDEYACRVNHVTLSQPKIVKWDRDMGGSM |
| | | KAIFVLNAQHDEAVDANSALAKAKVLANRELDKYGVSDYYKKLVNNAKTVEGVKDLQAQVVESAKKAR |
| | | GGSHHHHHHLE |
| 672 | S022 | MKAIFVLNAQHDEAVDANSALAKAKVLANRELDKYGVSDYYKKLVNNAKTVEGVKDLQAQVVESAKK |
| | | ARGGSMIKEEHVIIQAEFYLNPDQSGEFMFDFDGDEIFHVDMAKKETVWRLEEFGRFASFEAQGALANIAV |
| | | DKANLEIMTKRSNYTPLEMGDTRPRFLWQLKFECHFFNGTERVRLLERCIYNQEESVRFDSDVGEYRAVTE |
| | | LGRPDAEYWNSQKDLLEQRRAAVDTYCRHNYGVGESFTVQGGSQRTPKIQVYSRHPAENGKSNFLNCYV |
| | | SGFHPSDIEVDLLKNGERIEKVEHSDLSFSKDWSFYLLYYTEFTPTEKDEYACRVNHVTLSQPKIVKWDRD |
| | | MGGSHHHHHHLE |
| 673 | S023 | MSDSEVNQEAKPEVKPEVKPETHINLKVSDGSSEIFFKIKKTTPLRRLMEAFAKRQGKEMDSLRFLYDGIRI |
| | | QADQTPEDLDMEDNDIIEAHREQIGGMIKEEHVIIQAEFYLNPDQSGEFMFDFDGDEIFHVDMAKKETVWR |
| | | LEEFGRFASFEAQGALANIAVDKANLEIMTKRSNYTPLEMGDTRPRFLWQLKFECHFFNGTERVRLLERCIY |
| | | NQEESVRFDSDVGEYRAVTELGRPDAEYWNSQKDLLEQRRAAVDTYCRHNYGVGESFTVQGGSHHHHH |
| | | HLE |
| 674 | S024 | MSEDDEEKEEDGMIKEEHVIIQAEFYLNPDQSGEFMFDFDGDEIFHVDMAKKETVWRLEEFGRFASFEAQG |
| | | ALANIAVDKANLEIMTKRSNYTPLEMGDTRPRFLWQLKFECHFFNGTERVRLLERCIYNQEESVRFDSDVG |
| | | EYRAVTELGRPDAEYWNSQKDLLEQRRAAVDTYCRHNYGVGESFTVQGGSHHHHHHLE |
| 675 | S025 | MSEDDEEKEEDGLVGSHSMRYFYTSVSRPGRGEPRFISVGYVDDTQFVRFDSDAASPREEPRAPWIEQEGPE |
| | | YWDRNTQIYKAQAQTDRESLRNLRGYYNQSEAGSHTLQSMYGCDVGPDGRLLRGHDQYAYDGKDYIAL |
| | | NEDLRSWTAADTAAQITQRKWEAAREAEQRRAYLEGECVEWLRRYLENGKDKLERADPPKTHVTHHPIS |
| | | DHEATLRCWALGFYPAEITLTWQRDGEDQTQDTELVETRPAGDRTFQKWAAVVVPSGEEQRYTCHVQHE |
| | | GLPKPLTLRWEPSSQSTVPIVGIVAGLAVLAVVVIGAVVAAVMCRRKSSGGKGGSYSQAACSDSAQGSDVS |
| | | LTAGGSHHHHHH |
| 676 | S026 | MGSHSMRYFYTSVSRPGRGEPRFISVGYVDDTQFVRFDSDAASPREEPRAPWIEQEGPEYWDRNTQIYKAQ |
| | | AQTDRESLRNLRGYYNQSEAGSHTLQSMYGCDVGPDGRLLRGHDQYAYDGKDYIALNEDLRSWTAADT |
| | | AAQITQRKWEAAREAEQRRAYLEGECVEWLRRYLENGKDKLERADPPKTHVTHHPISDHEATLRCWALG |
| | | FYPAEITLTWQRDGEDQTQDTELVETRPAGDRTFQKWAAVVVPSGEEQRYTCHVQHEGLPKPLTLRWEPS |
| | | SQSTVPIVGIVAGLAVLAVVVIGAVVAAVMCRRKSSGGKGGSYSQAACSDSAQGSDVSLTAGGSHHHHHH |
| 677 | S027 | MGSHSMRYFYTSVSRPGRGEPRFISVGYVDDDQFVRFDSDAASPREEPRAPWIEQEGPEYWDRNTQIYKAQ |
| | | AQTDRESLRNLRGYYNQSEAGSHTLQSMYGCDVGPDGRLLRGHDQYAYDGKDYIALNEDLRSWTAADT |
| | | AAQITQRKWEAAREAEQRRAYLEGECVEWLRRYLENGKDKLERADGGSHHHHHH |
| 678 | S028 | MGSHSMRYFYTSVSRPGRGEPRFISVGYVDGDQFVRFDSDAASPREEPRAPWIEQEGPEYWDRNTQIYKAQ |
| | | AQTDRESLRNLRGYYNQSEAGSHTLQSMYGCDVGPDGRLLRGHDQYAYDGKDYIALNEDLRSWTAADT |
| | | AAQITQRKWEAAREAEQRRAYLEGECVEWLRRYLENGKDKLERADGGSHHHHHH |
| 679 | S029 | MGSHSMRYFYTSVSRPGRGEPRFISVGYVDGDEIFHVDSDAASPREEPRAPWIEQEGPEYWDRNTQIYKAQ |
| | | AQTDRESLRNLRGYYNQSEAGSHTLQSMYGCDVGPDGRLLRGHDQYAYDGKDYIALNEDLRSWTAADT |
| | | AAQITQRKWEAAREAEQRRAYLEGECVEWLRRYLENGKDKLERADGGSHHHHHH |
| 680 | S030 | MGSHSMRYFYTSVSRPGRGEPRFISVGYVDGDEIFHVDSDAASPREEPRAPWIEQEGPSYGDRNTQIYKAQ |
| | | AQTDRESLRNLRGYYNQSEAGSHTLQSMYGCDVGPDGRLLRGHDQYAYDGKDYIALNEDLRSWTAADT |
| | | AAQITQRKWEAAREAEQRRAYLEGECVEWLRRYLENGKDKLERADGGSHHHHHH |
| 681 | S031 | MGSHSMRYFYTSVSRPGRGEPRFISVGYVDGDEIFHVDSDAASPREEPRAPWIEQEGPSYGDRNTQIYKAQ |
| | | AQTDRESLRNLRGYYNQSEAGSHTLQSMYGCDVGPDGRLLRGHDQYAYDGKDYIALNEDLRSWTAADT |
| | | AAQITQRKWETNREAEQRRAYLEGECVEWLRRYLENGKDKLERADGGSHHHHHH |
| 682 | S032 | MGSHSMRYFYTSVSRPGRGEPRFISVGYVDGDEIFHVDSDAASPREEPRAPWIEQEGPSYGDRNTQIYKAQ |
| | | AQTDRESLRNLRGYYNQSEAGSHTLQSMYGCDVGPDGRLLRGHDQYAYDGKDYIALNEDLRSWTAADT |
| | | AAQITQRKWETQKEAEQRRAYLEGECVEWLRRYLENGKDKLERADGGSHHHHHH |
| 683 | S033 | MGSHSMRYFYTSVSRPGRGEPRFISVGYVDGDEIFHVDSDAASPREEPRAPWIEQEGPSYGDRNTQIYKAQ |
| | | AQTDRESLRNLRGYYNQSEAGSHTLQSMYGCDVGPDGRLLRGHDQYAYDGKDYIALNEDLRSWTAADT |
| | | AAQITQRKWETQKDLLQRRAYLEGECVEWLRRYLENGKDKLERADGGSHHHHHH |
| 684 | S034 | MGSHSMRYFYTSVSRPGRGEPRFISVGYVDGDEIFHVDSDAASPREEPRLEEFEQEGPSYGDRNTQIYKAQA |
| | | QTDRESLRNLRGYYNQSEAGSHTLQSMYGCDVGPDGRLLRGHDQYAYDGKDYIALNEDLRSWTAADTA |
| | | AQITQRKWETQKEAEQRRAYLEGECVEWLRRYLENGKDKLERADGGSHHHHHH |
| 685 | S035 | MGSHSMRYFYTSVSRPGRGEPRFISVGYVDGDEIFHVDSDAASPREEPRLEEFEQEGPSYGDRNTQIYKAQA |
| | | QTDRESLRNLRGYYNQSEAGSHTLQSMYGCDVGPDGRLLRGHDQYAYDGKDYIALNEDLRSWTAADTA |
| | | AQITQRKWETQKEAEQRRAYLEGECVEWLRRYLENGKDKLERADQRTPKIQVYSRHPAENGKSNFLNCY |
| | | VSGFHPSDIEVDLLKNGERIEKVEHSDLSFSKDWSFYLLYYTEFTPTEKDEYACRVNHVTLSQPKIVKWDRD |
| | | MGGSMKAIFVLNAQHDEAVDANSLAEAKVLANRELDKYGVSDYYKNLINNAKTVEGVKALIDEILAALPG |
| | | GSENLYFQGGSHHHHHHLE |
| 686 | S036 | MGSHSMRYFYTSVSRPGRGEPRFISVGYVDGDEIFHVDSDAASPREEPRLEEFEQEGPSYGDRNTQIYKAQA |
| | | QTDRESLRNLRGYYNQSEAGSHTLQSMYGCDVGPDGRLLRGHDQYAYDGKDYIALNEDLRSWTAADTA |
| | | AQITQRKWETQKEAEQRRAYLEGECVEWLRRYLENGKDKLERADQRTPKIQVYSRHPAENGKSNFLNCY |
| | | VSGFHPSDIEVDLLKNGERIEKVEHSDLSFSKDWSFYLLYYTEFTPTEKDEYACRVNHVTLSQPKIVKWDRD |
| | | MGGSHHHHHHLE |
| 687 | S037 | MGSHSMRYFYTSVSRPGRGEPRFISVGYVDGDEIFHVDSDAASPREEPRLEEFEQEGPSYGDRNTQIYKAQA |
| | | QTDRESLRNLRGYYNQSEAGSHTLQSMYGCDVGPDGRLLRGHDQYAYDGKDYIALNEDLRSWTAADTA |
| | | AQITQRKWETQKEAEQRRAYLEGECVEWLRRYLENGKDKLERADMKAIFVLNAQHDEAVDANSLAEAK |
| | | VLANRELDKYGVSDYYKNLINNAKTVEGVKALIDEILAALPGGSHHHHHHLE |
| 688 | S038 | MGSHSMRYFYTSVSRPGRGEPRFISVGYVDGDEIFHVDSDAASPREEPRLEEFEQEGPSYGDRNTQIYKAQA |
| | | QTDRESLRNLRGYYNQSEAGSHTLQSMYGCDVGPDGRLLRGHDQYAYDGKDYIALNEDLRSWTAADTA |
| | | AQITQRKWETQKEAEQRRAYLEGECVEWLRRYLENGKDKLERADMKAIFVLNAQHDEAVDANSLAEAK |
| | | VLANRELDKYGVSDYYKNLINNAKTVEGVKALIDEILAALPGGSHHHHHHLESEDDEEKEEDG |
| 689 | S039 | MSDSEVNQEAKPEVKPEVKPETHINLKVSDGSSEIFFKIKKTTPLRRLMEAFAKRQGKEMDSLRFLYDGIRI |
| | | QADQTPEDLDMEDNDIIEAHREQIGGLVMAPRTVLLLLSAALALTETWAGSHSMRYFYTSVSRPGRGEPRF |
| | | ISVGYVDDDQFVRFDSDAASPREEPRAPWIEQEGPEYWDRNTQIYKAQAQTDRESLRNLRGYYNQSEAGS |
| | | HTLQSMYGCDVGPDGRLLRGHDQYAYDGKDYIALNEDLRSWTAADTAAQITQRKWEAAREAEQRRAYL |
| | | EGECVEWLRRYLENGKDKLERADGGSHHHHHH |
| 690 | S040 | MGSHSMRYFYTSVSRPGRGEPRFISVGYVDDTQFVRFDSDAASPREEPRAPWIEQEGPEYWDRNTQIYKAQ |
| | | AQTDRESLRNLRGYYNQSEAGSHTLQSMYGCDVGPDGRLLRGHDQYAYDGKDYIALNEDLRSWTAADT |
| | | AAQITQRKWEAAREAEQRRAYLEGECVEWLRRYLENGKDKLERADPPKTHVTHHPISDHEATLRCWALG |
| | | FYPAEITLTWQRDGEDQTQDTELVETRPAGDRTFQKWAAVVVPSGEEQRYTCHVQHEGLPKPLTLRWEPS |
| | | SQSTVPIVGIVAGLAVLAVVVIGAVVAAVMCRRKSSGGKGGSYSQAACSDSAQGSDVSLTAGGSHHHHHH |
| | | SEDDEEKEEDG |
| 691 | S041 | MIKEEHVIIQAEFYLNPDQSGEFMFDFDGDEIFHVDMAKKETVWRLEEFEQEGPSYGDRNTQIYKAQAQTD |
| | | RESLRNLRGYYNQSEAGSHTLQSMYGCDVGPDGRLLRGHDQYAYDGKDYIALNEDLRSWTAADTAAQIT |
| | | QRKWETQKEAEQRRAYLEGECVEWLRRYLENGKDKLERADPPKTHVTHHPISDHEATLRCWALGFYPAEI |
| | | TLTWQRDGEDQTQDTELVETRPAGDRTFQKWAAVVVPSGEEQRYTCHVQHEGLPKPLTLRWEPSSQSTVP |
| | | IVGIVAGLAVLAVVVIGAVVAAVMCRRKSSGGKGGSYSQAACSDSAQGSDVSLTAGGSHHHHHHLE |
| 692 | S042 | MIKEEHVIIQAEFYLNPDQSGEFMFDFDGDEIFHVDMAKKETVWRLEEFEQEGPSYGDRNTQIYKAQAQTD |
| | | RESLRNLRGYYNQSEAGSHFLWQLKFECHFFNGTERVRLLERCIYDGKDYIALNEDLRSWTAADTAAQITQ |
| | | RKWETQKEAEQRRAYLEGECVEWLRRYLENGKDKLERADPPKTHVTHHPISDHEATLRCWALGFYPAEIT |
| | | LTWQRDGEDQTQDTELVETRPAGDRTFQKWAAVVVPSGEEQRYTCHVQHEGLPKPLTLRWEPSSQSTVPI |
| | | VGIVAGLAVLAVVVIGAVVAAVMCRRKSSGGKGGSYSQAACSDSAQGSDVSLTAGGSHHHHHHLE |
| 693 | S043 | MIKEEHVIIQAEFYLNPDQSGEFMFDFDGDEIFHVDMAKKETVWRLEEFEQEGPSYGDRNTQIYKAQAQTD |
| | | RESLRNLRGYYNQSEAGSHFLWQLKFECHFFNGTERVRLLERCIYDGKDYIALNEDLRSWTAADTAAQITQ |
| | | RKWETQKEAEQRRAYLEGECVEWLRRYLENGKDKLERADGGSHHHHHHLE |
| 694 | S044 | MIKEEHVIIQAEFYLNPDQSGEFMFDFDGDEIFHVDMAKKETVWRLEEFEQEGPSYGDRNTQIYKAQAQTD |
| | | RESLRNLRGYYNQSEAGSHFLWQLKFECHFFNGTERVRLLERCIYDGKDYIALNEDLRSWTAADTAAQITQ |
| | | RKWETQKEAEQRRAYLEGECVEWLRRYLENGKDKLERADQRTPKIQVYSRHPAENGKSNFLNCYVSGFH |
| | | PSDIEVDLLKNGERIEKVEHSDLSFSKDWSFYLLYYTEFTPTEKDEYACRVNHVTLSQPKIVKWDRDMGGS |
| | | HHHHHHLE |
| 695 | S045 | IQRTPKIQVYSRHPAENGKSNFLNCYVSGFHPSDIEVDLLKNGERIEKVEHSDLSFSKDWSFYLLYYTEFTPT |
| | | EKDEYACRVNHVTLSQPKIVKWDRDMGGSGGSGGSGGSGSHSMRYFYTSVSRPGRGEPRFISVGYVDDTQ |
| | | FVRFDSDAASPREEPRAPWIEQEGPEYWDRNTQIYKAQAQTDRESLRNLRGYYNQSEAGSHTLQSMYGCD |
| | | VGPDGRLLRGHDQYAYDGKDYIALNEDLRSWTAADTAAQITQRKWEAAREAEQRRAYLEGECVEWLRR |
| | | YLENGKDKLERADPPKTHVTHHPISDHEATLRCWALGFYPAEITLTWQRDGEDQTQDTELVETRPAGDRTF |
| | | QKWAAVVVPSGEEQRYTCHVQHEGLPKPLTLRWEGGSHHHHHHLE |
| 696 | S046 | IQRTPKIQVYSRHPAENGKSNFLNCYVSGFHPSDIEVDLLKNGERIEKVEHSDLSFSKDWSFYLLYYTEFTPT |
| | | EKDEYACRVNHVTLSQPKIVKWDRDMTSGGGGSGGGGSGGGGSGGGGSSSGSHSMRYFYTSVSRPGRGE |
| | | PRFISVGYVDDTQFVRFDSDAASPREEPRAPWIEQEGPEYWDRNTQIYKAQAQTDRESLRNLRGYYNQSEA |
| | | GSHTLQSMYGCDVGPDGRLLRGHDQYAYDGKDYIALNEDLRSWTAADTAAQITQRKWEAAREAEQRRA |
| | | YLEGECVEWLRRYLENGKDKLERADPPKTHVTHHPISDHEATLRCWALGFYPAEITLTWQRDGEDQTQDT |
| | | ELVETRPAGDRTFQKWAAVVVPSGEEQRYTCHVQHEGLPKPLTLRWEGGSHHHHHHLE |
| 697 | S047 | GSHSMRYFYTSVSRPGRGEPRFISVGYVDDTQFVRFDSDAASPREEPRAPWIEQEGPEYWDRNTQIYKAQA |
| | | QTDRESLRNLRGYYNQSEAGSHTLQSMYGCDVGPDGRLLRGHDQYAYDGKDYIALNEDLRSWTAADTA |
| | | AQITQRKWEAAREAEQRRAYLEGECVEWLRRYLENGKDKLERADPPKTHVTHHPISDHEATLRCWALGF |
| | | YPAEITLTWQRDGEDQTQDTELVETRPAGDRTFQKWAAVVVPSGEEQRYTCHVQHEGLPKPLTLRWESGG |
| | | GGSGGGGSGGGGSGGGGIQRTPKIQVYSRHPAENGKSNFLNCYVSGFHPSDIEVDLLKNGERIEKVEHSDLS |
| | | FSKDWSFYLLYYTEFTPTEKDEYACRVNHVTLSQPKIVKWDRDMGGSHHHHHHLE |
| 698 | S048 | GSHSMRYFYTSVSRPGRGEPRFISVGYVDDTQFVRFDSDAASPREEPRAPWIEQEGPEYWDRNTQIYKAQA |
| | | QTDRESLRNLRGYYNQSEAGSHTLQSMYGCDVGPDGRLLRGHDQYAYDGKDYIALNEDLRSWTAADTA |
| | | AQITQRKWEAAREAEQRRAYLEGECVEWLRRYLENGKDKLERADPPKTHVTHHPISDHEATLRCWALGF |
| | | YPAEITLTWQRDGEDQTQDTELVETRPAGDRTFQKWAAVVVPSGEEQRYTCHVQHEGLPKPLTLRWESGG |
| | | GGSGGGGSGGGGSGGGGSGGGGSGGGGIQRTPKIQVYSRHPAENGKSNFLNCYVSGFHPSDIEVDLLKNGE |
| | | RIEKVEHSDLSFSKDWSFYLLYYTEFTPTEKDEYACRVNHVTLSQPKIVKWDRDMGGSHHHHHHLE |
| 699 | S049 | GSHSMRYFYTSVSRPGRGEPRFISVGYVDDTQFVRFDSDAASPREEPRAPWIEQEGPEYWDRNTQIYKAQA |
| | | QTDRESLRNLRGYYNQSEAGSHTLQSMYGCDVGPDGRLLRGHDQYAYDGKDYIALNEDLRSWTAADTA |
| | | AQITQRKWEAAREAEQRRAYLEGECVEWLRRYLENGKDKLERADSGGGGSGGGGSGGGGIQRTPKIQVY |
| | | SRHPAENGKSNFLNCYVSGFHPSDIEVDLLKNGERIEKVEHSDLSFSKDWSFYLLYYTEFTPTEKDEYACRV |
| | | NHVTLSQPKIVKWDRDMGGSHHHHHHLE |
| 700 | S050 | GSHSMRYFYTSVSRPGRGEPRFISVGYVDDTQFVRFDSDAASPREEPRAPWIEQEGPEYWDRNTQIYKAQA |
| | | QTDRESLRNLRGYYNQSEAGSHTLQSMYGCDVGPDGRLLRGHDQYAYDGKDYIALNEDLRSWTAADTA |
| | | AQITQRKWEAAREAEQRRAYLEGECVEWLRRYLENGKDKLERADTSGGGGSGGGGSGGGGSGGGGSSSI |
| | | QRTPKIQVYSRHPAENGKSNFLNCYVSGFHPSDIEVDLLKNGERIEKVEHSDLSFSKDWSFYLLYYTEFTPTE |
| | | KDEYACRVNHVTLSQPKIVKWDRDMGGSHHHHHHLE |
| 701 | S051 | IQRTPKIQVYSRHPAENGKSNFLNCYVSGFHPSDIEVDLLKNGERIEKVEHSDLSFSKDWSFYLLYYTEFTPT |
| | | EKDEYACRVNHVTLSQPKIVKWDRDMSGGGGSGGGGSGGGGGSHSMRYFYTSVSRPGRGEPRFISVGYV |
| | | DDTQFVRFDSDAASPREEPRAPWIEQEGPEYWDRNTQIYKAQAQTDRESLRNLRGYYNQSEAGSHTLQSM |
| | | YGCDVGPDGRLLRGHDQYAYDGKDYIALNEDLRSWTAADTAAQITQRKWEAAREAEQRRAYLEGECVE |
| | | WLRRYLENGKDKLERADGGSHHHHHHLE |
| 702 | S052 | IQRTPKIQVYSRHPAENGKSNFLNCYVSGFHPSDIEVDLLKNGERIEKVEHSDLSFSKDWSFYLLYYTEFTPT |
| | | EKDEYACRVNHVTLSQPKIVKWDRDMTSGGGGSGGGGSGGGGSGGGGSSSGSHSMRYFYTSVSRPGRGE |
| | | PRFISVGYVDDTQFVRFDSDAASPREEPRAPWIEQEGPEYWDRNTQIYKAQAQTDRESLRNLRGYYNQSEA |
| | | GSHTLQSMYGCDVGPDGRLLRGHDQYAYDGKDYIALNEDLRSWTAADTAAQITQRKWEAAREAEQRRA |
| | | YLEGECVEWLRRYLENGKDKLERADGGSHHHHHHLE |
| 703 | S053 | CGGGSHSMRYFYTSVSRPGRGEPRFISVGYVDDTQFVRFDSDAASPREEPRAPWIEQEGPEYWDRNTQIYK |
| | | AQAQTDRESLRNLRGYYNQSEAGSHTLQSMYGCDVGPDGRLLRGHDQYAYDGKDYIALNEDLRSWTAA |
| | | DTAAQITQRKWEAAREAEQRRAYLEGECVEWLRRYLENGKDKLERADGGCGGSHHHHHHLE |
| 704 | S054 | GSHSMRYYYTSVSRPGRGEPRFNSTGYVDDTQFVRFDSDAASPREEPRAPWIEQEGPEYWDRNTQIYKAQ |
| | | AQTDRESLRNLRGYYNQSEAGSHTLQSKYGCDVGPDGRLLRGHDQYAYDGKDYIALNEDLRSWTAADTA |
| | | AQITQRKWEAAREAEQRRAYLEGECVEWLRRYLENGKDKLERADGGSHHHHHHLE |
| 705 | S055 | GSHSMRYNYTSVSRPGRGEPRFLSEGHVDDTQFVRFDSDAASPREEPRAPWIEQEGPEYWDRNTQIYKAQA |
| | | QTDRESLRNLRGYYNQSEAGSHTLQSIYGCDVGPDGRLLRGHDQYYYDGKDYISLNEDLRSWTAADTAA |
| | | QITQRKWEAAREAEQRRAYLEGECVEWLRRYLENGKDKLERADGGSHHHHHHLE |
| 706 | S056 | GSHSMRYNYTSVSRPGRGEPRFLSEGHVDGQPFVRFDSDAASPREEPRAPWIEQEGPEYWDRNTQIYKAQA |
| | | QTDRESLRNLRGYYNQSEAGLHSLNSIYVCDVGPDGRLLRGHDHYYYDGELYISLNEDLRSWTAADTAAQ |
| | | ITQRKWEAAREAEQRRAYLEGECVEWLRRYLENGKDKLERADGGSHHHHHHLE |
| 707 | S057 | EPHSLRYNLTVLSWDGSVQSGFLTEVHLDGQPFLRCDRQKCRAKPQGQWAEDVLGNKTWDRNTQIYKAQ |
| | | AQTDRESLRNLRGYYNQKEGLHSLQEIRVCEIHEDNSTRSSQHFYYDGELFLSQNLETKEWTMPQSSRADT |
| | | AAQITQRKWEAAREAEQRRAYLEGECVEWLRRYLENGKDKLERADGGSHHHHHHLE |
| 708 | S058 | GSHSMRYNYTSVSRPGRGEPRFISEGYVDDTQFVRFDSDAASPREEPRAPWIEQEGPEYWDRNTQIYKAQA |
| | | QTDRESLRNLRGYYNQSEAGSHTLQSMYGCDVGPDGRLLRGHDQYYYDGKDYISLNEDLRSWTAADTAA |
| | | QITQRKWEAAREAEQRRAYLEGECVEWLRRYLENGKDKLERADGGSHHHHHHLE |
| 709 | S059 | GSHSMRYNYMSLSRPGRGEPRFLSEGHVDGTPFVRFDSDAASPREEPRAPWIEQEGPEYWDRNTQIYKAQA |
| | | QTDRESLRNLRGYYNQSEAGLHSLQSIYVCEVGPDGRLLRGSDHYYYNGKLYISLNEDLRSWTAADTAAQI |
| | | TQRKWEAAREAEQRRAYLEGECVEWLRRYLENGKDKLERADGGSHHHHHHLE |
| 710 | S060 | EPHSLRYNLMVLSQDGSVQSGFLAEGHLDGQPFLRYDRQKRRAKPRAPWIEQEGPEYWDRNTQIYKAQAQ |
| | | TDRESLRNLRGYYNQSEAGSHSLQEIRVCEIHEDSSTRGSRHFYYNGELFLSQNLETQESTAADTAAQITQR |
| | | KWEAAREAEQRRAYLEGECVEWLRRYLENGKDKLERADGGSHHHHHHLE |
| 711 | S061 | GSHSMRYFYTSVSRPGRGEPRFISVGYVDDTQFVRFDSDAASPREEPRAPWIEQEGPESWDRNTQIYKAQA |
| | | QTDRESLRNLRGRYNQSEAGSHTLQSMYGCDVGPDGRLLRGHDQYAYDGKDYIALNEDLRSWTAADTAA |
| | | QPTQRYWEAAREAEQRRAYLEGECVEHLRRYLENGKDKLERADGGSHHHHHHLE |
| 712 | S062 | GSHSMRYFYTSVSRPGRGEPRFISVGYVDDTQFVRFDSDAASPREEPRAPWIEQSGPESWDRNTQIYKAQA |
| | | QTDRESLRTLRGRYNQSEAGSHTLQSMYGCDVGPDGRLLRGHDQYAYDGKDFIALNEDLRSWTAADTAA |
| | | QPTQRYWEAAREAEQRRAYLEGECVEHLRLYLENGKDKLERADGGSHHHHHHLE |
| 713 | S063 | GSHSMRYFYTSVSRPGRGEPRFISVGYVDDTQFVRFDSDAASPREEPRAPWIEQEGPEGWDRNTQGYKAQA |
| | | QTDRESLRNMRGYYNQSEAGSHTLQSMYGCDVGPDGRLLRGHDQYAYDGKDYIALNEDLRSWTAADTA |
| | | AQIDQRKWEAAREAEQRRAYLEGECVEWLRRYLENGKDKLERADGGSHHHHHHLE |
| 714 | S064 | GSHSMRYFYTSVSRPGRGEPRFISVGYVDDTQFVRFDSDAASPREEPRAPWIEQEGPEGWDRNPQGYKAQA |
| | | QTDRESLRNMRGRYNQSEAGSHTLQSMYGCDVGPDGRLLRGHDQYAYDGKDYIALNEDLRSWTAADTA |
| | | AQIDQRYWEAAREAEQRRAYLEGECVEWLRRYLENGKDKLERADGGSHHHHHHLE |
| 715 | S065 | GSHSMRYFYTSVSRPGRGEPRFISVGYVDDTQFVRFDSDAASPREEPRAPWIEQEGPEAWDRNTQIYKAQA |
| | | QTDRESLRNLRGYYNQSEAGSHTLQSMYGCDVGPDGRLLRGHDQYAYDGKDSIALNEDLRSWTAADTAA |
| | | QITQRYWEAAREAEQRRAYLEGECVEHLRRVLENGKDKLERADGGSHHHHHHLE |
| 716 | S066 | GSHSMRYFYTSVSRPGRGEPRFISVGYVDDTQIVRFDSDAASPREEPRAPFIEQAGPEAWDRNTQIYKAQAQ |
| | | TDRASLRILRGYYNQSEAGSHTLQSMYGCDVGPDGRLLRGHDQYAYDGKDSIALNEDLRSWTAADTAAQI |
| | | TQRYWEAAREAEQRRAYLEGECVEHLRGVLENGKDKLERADGGSHHHHHHLE |
| 717 | S067 | KPVSKMRMATPLLMQALPMGGGGSGGGGGDTRPRFLWQLKFECHFFNGTERVRLLERCIYNQEESVRFDS |
| | | DVGEYRAVTELGRPDAEYWNSQKDLLEQRRAAVDTYCRHNYGVGESFTVQRRVEPKVTVYPSKTQPLQH |
| | | HNLLVCSVSGFYPGSIEVRWFRNGQEEKAGVVSTGLIQNGDWTFQTLVMLETVPRSGEVYTCQVEHPSVTS |
| | | PLTVEWRASGGGGSGGGGSGGGGIKEEHVIIQAEFYLNPDQSGEFMFDFDGDEIFHVDMAKKETVWRLEEF |
| | | GRFASFEAQGALANIAVDKANLEIMTKRSNYTPITNVPPEVTVLTNSPVELREPNVLICFIDKFTPPVVNVTW |
| | | LRNGKPVTTGVSETVFLPREDHLFRKFHYLPFLPSTEDVYDCRVEHWGLDEPLLKHWEFDAPSGGSHHHH |
| | | HHLE |
| 718 | S068 | GDTRPRFLWQLKFECHFFNGTERVRLLERCIYNQEESVRFDSDVGEYRAVTELGRPDAEYWNSQKDLLEQ |
| | | RRAAVDTYCRHNYGVGESFTVQRRVEPKVTVYPSKTQPLQHHNLLVCSVSGFYPGSIEVRWFRNGQEEKA |
| | | GVVSTGLIQNGDWTFQTLVMLETVPRSGEVYTCQVEHPSVTSPLTVEWRASGGGGSGGGGSGGGGIKEEH |
| | | VIIQAEFYLNPDQSGEFMFDFDGDEIFHVDMAKKETVWRLEEFGRFASFEAQGALANIAVDKANLEIMTKR |
| | | SNYTPITNVPPEVTVLTNSPVELREPNVLICFIDKFTPPVVNVTWLRNGKPVTTGVSETVFLPREDHLFRKFH |
| | | YLPFLPSTEDVYDCRVEHWGLDEPLLKHWEFDAPSGGSHHHHHHLE |
| 719 | S069 | GDTRPRFLWQLKFECHFFNGTERVRLLERCIYNQEESVRFDSDVGEYRAVTELGRPDAEYWNSQKDLLEQ |
| | | RRAAVDTYCRHNYGVGESFTVQRRVEPKVTVYPSKTQPLQHHNLLVCSVSGFYPGSIEVRWFRNGQEEKA |
| | | GVVSTGLIQNGDWTFQTLVMLETVPRSGEVYTCQVEHPSVTSPLTVEWRATSGGGGSGGGGSGGGGSGGG |
| | | GSSSIKEEHVIIQAEFYLNPDQSGEFMFDFDGDEIFHVDMAKKETVWRLEEFGRFASFEAQGALANIAVDKA |
| | | NLEIMTKRSNYTPITNVPPEVTVLTNSPVELREPNVLICFIDKFTPPVVNVTWLRNGKPVTTGVSETVFLPRE |
| | | DHLFRKFHYLPFLPSTEDVYDCRVEHWGLDEPLLKHWEFDAPSGGSHHHHHHLE |
| 720 | S070 | IKEEHVIIQAEFYLNPDQSGEFMFDFDGDEIFHVDMAKKETVWRLEEFGRFASFEAQGALANIAVDKANLEI |
| | | MTKRSNYTPITNVPPEVTVLTNSPVELREPNVLICFIDKFTPPVVNVTWLRNGKPVTTGVSETVFLPREDHLF |
| | | RKFHYLPFLPSTEDVYDCRVEHWGLDEPLLKHWEFDAPSSGGGGSGGGGSGGGGGDTRPRFLWQLKFECH |
| | | FFNGTERVRLLERCIYNQEESVRFDSDVGEYRAVTELGRPDAEYWNSQKDLLEQRRAAVDTYCRHNYGVG |
| | | ESFTVQRRVEPKVTVYPSKTQPLQHHNLLVCSVSGFYPGSIEVRWFRNGQEEKAGVVSTGLIQNGDWTFQT |
| | | LVMLETVPRSGEVYTCQVEHPSVTSPLTVEWRAGGSHHHHHHLE |
| 721 | S071 | IKEEHVIIQAEFYLNPDQSGEFMFDFDGDEIFHVDMAKKETVWRLEEFGRFASFEAQGALANIAVDKANLEI |
| | | MTKRSNYTPITNVPPEVTVLTNSPVELREPNVLICFIDKFTPPVVNVTWLRNGKPVTTGVSETVFLPREDHLF |
| | | RKFHYLPFLPSTEDVYDCRVEHWGLDEPLLKHWEFDAPSTSGGGGSGGGGSGGGGSGGGGSSSGDTRPRF |
| | | LWQLKFECHFFNGTERVRLLERCIYNQEESVRFDSDVGEYRAVTELGRPDAEYWNSQKDLLEQRRAAVDT |
| | | YCRHNYGVGESFTVQRRVEPKVTVYPSKTQPLQHHNLLVCSVSGFYPGSIEVRWFRNGQEEKAGVVSTGLI |
| | | QNGDWTFQTLVMLETVPRSGEVYTCQVEHPSVTSPLTVEWRAGGSHHHHHHLE |
| 722 | S072 | IKEEHVIIQAEFYLNPDQSGEFMFDFDGDEIFHVDMAKKETVWRLEEFGRFASFEAQGALANIAVDKANLEI |
| | | MTKRSNYTPITNVPPEVTVLTNSPVELREPNVLICFIDKFTPPVVNVTWLRNGKPVTTGVSETVFLPREDHLF |
| | | RKFHYLPFLPSTEDVYDCRVEHWGLDEPLLKHWEFDAPSSGGGGSGGGGSGGGGGDTRPRFLWQLKFECH |
| | | FFNGTERVRLLERCIYNQEESVRFDSDVGEYRAVTELGRPDAEYWNSQKDLLEQRRAAVDTYCRHNYGVG |
| | | ESFTVQRRGGSHHHHHHLE |
| 723 | S073 | IKEEHVIIQAEFYLNPDQSGEFMFDFDGDEIFHVDMAKKETVWRLEEFGRFASFEAQGALANIAVDKANLEI |
| | | MTKRSNYTPITNVPPEVTVLTNSPVELREPNVLICFIDKFTPPVVNVTWLRNGKPVTTGVSETVFLPREDHLF |
| | | RKFHYLPFLPSTEDVYDCRVEHWGLDEPLLKHWEFDAPSSGGGGSGGGGSGGGGGDTRPRFLWQLKFECH |
| | | FFNGTERVRLLERCIYNQEESVRFDSDVGEYRAVTELGRPDAEYWNSQKDLLEQRRAAVDTYCRHNYGVG |
| | | ESFTVQRRGGSGGSKPVSKMRMATPLLMQALPMGGSHHHHHHLE |
| 724 | S074 | GDTRPRFLWQLKFECHFFNGTERVRLLERCIYNQEESVRFDSDVGEYRAVTELGRPDAEYWNSQKDLLEQ |
| | | RRAAVDTYCRHNYGVGESFTVQRRASGGGGSGGGIKEEHVIIQAEFYLNPDQSGEFMFDFDGDEIFHVDM |
| | | AKKETVWRLEEFGRFASFEAQGALANIAVDKANLEIMTKRSNYTPITNVPPEVTVLTNSPVELREPNVLICFI |
| | | DKFTPPVVNVTWLRNGKPVTTGVSETVFLPREDHLFRKFHYLPFLPSTEDVYDCRVEHWGLDEPLLKHWE |
| | | FDAPSGGSHHHHHHLE |
| 725 | S075 | KPVSKMRMATPLLMQALPMASGGGGSGGGGDTRPRFLWQLKFECHFFNGTERVRLLERCIYNQEESVRFD |
| | | SDVGEYRAVTELGRPDAEYWNSQKDLLEQRRAAVDTYCRHNYGVGESFTVQRRASGGGGSGGGIKEEHV |
| | | IIQAEFYLNPDQSGEFMFDFDGDEIFHVDMAKKETVWRLEEFGRFASFEAQGALANIAVDKANLEIMTKRS |
| | | NYTPITNVPPEVTVLTNSPVELREPNVLICFIDKFTPPVVNVTWLRNGKPVTTGVSETVFLPREDHLFRKFHY |
| | | LPFLPSTEDVYDCRVEHWGLDEPLLKHWEFDAPSGGSHHHHHHLE |
| 726 | S076 | GDTRPRFLWQLKFECHFFNGTERVRLLERCIYNQEESVRFDSDVGEYRAVTELGRPDAEYWNSQKDLLEQ |
| | | RRAAVDTYCRHNYGVGESFTVQTRSGGGGSGGGGIKEEHVIIQAEFYLNPDQSGEFMFDFDGTEFFHVDM |
| | | AKKETVWRLEEWGRFASFEAQGALANIAVDKANLEIMTKRSNYTPITNVPPEVTVLTNSPVELREPNVLICF |
| | | IDKFTPPVVNVTWLRNGKPVTTGVSETVFLPREDHLFRKFHYLPFLPSTEDVYDCRVEHWGLDEPLLKHWE |
| | | FDAPSGGSHHHHHHLE |
| 727 | S077 | KPVSKMRMATPLLMQALPMASGGGGSGGGGDTRPRFLWQLKFECHFFNGTERVRLLERCIYNQEESVRFD |
| | | SDVGEYRAVTELGRPDAEYWNSQKDLLEQRRAAVDTYCRHNYGVGESFTVQRRSGGGIKEEHVIIQAEFY |
| | | LNPDQSGEFMFDFDGDEIFHVDMAKKETVWRLEEFGRFASFEAQGALANIAVDKANLEIMTKRSNYTGGS |
| | | HHHHHHLE |
| 728 | S078 | GDTRPRFLWQLKFECHFFNGTERVRLLERCIYNQEESVRFDSDVGEYRAVTELGRPDAEYWNSQKDLLEQ |
| | | RRAAVDTYCRHNYGVGESFTVQRRSGGGIKEEHVIIQAEFYLNPDQSGEFMFDFDGDEIFHVDMAKKETV |
| | | WRLEEFGRFASFEAQGALANIAVDKANLEIMTKRSNYTGGSHHHHHHLE |
| 729 | S079 | IKEEHVIIQAEFYLNPDQSGEFMFDFDGDEIFHVDMAKKETVWRLEEFGRFASFEAQGALANIAVDKANLEI |
| | | MTKRSNYTGDTRPRFLWQLKFECHFFNGTERVRLLERCIYNQEESVRFDSDVGEYRAVTELGRPDAEYWN |
| | | SQKDLLEQRRAAVDTYCRHNYGVGESFTVQRRGSGSGSKPVSKMRMATPLLMQALPMGGSHHHHHHLE |
| 730 | S080 | IKEEHVIIQAEFYLNPDQSGEFMFDFDGDEIFHVDMAKKETVWRLEEFGRFASFEAQGALANIAVDKANLEI |
| | | MTKRSNQSEAGSHLWQLKFECHFFNGTERVRLLERCIYNQEESVRFDSDVGEYRAVTELGRPDAEYWNSQ |
| | | KDLLEQRRAAVDTYCRHNYGVGESFTVQRRGGSHHHHHHLE |
| 731 | S081 | IKEEHVIIQAEFYLNPDQSGEFMFDFDGDEIFHVDMAKKETVWRLEEFGRFASFEAQGALANIAVDKANLEI |
| | | MTKRYNQSEAGSFLWQLKFECHFFNGTERVRLLERCIYNQEESVRFDSDVGEYRAVTELGRPDAEYWNSQ |
| | | KDLLEQRRAAVDTYCRHNYGVGESFTVQRRGGSHHHHHHLE |
| 732 | S082 | IKEEHVIIQAEFYLNPDQSGEFMFDFDGDEIFHVDMAKKETVWRLEEFGRFASFEAQGALANIAVDKANLEI |
| | | MTKRSNSGGGGFLWQLKFECHFFNGTERVRLLERCIYNQEESVRFDSDVGEYRAVTELGRPDAEYWNSQK |
| | | DLLEQRRAAVDTYCRHNYGVGESFTVQRRGGSHHHHHHLE |
| 733 | S083 | IKEEHVIIQAEFYLNPDQSGEFMFDFDGDEIFHVDMAKKETVWRLEEFGRFASFEAQGALANIAVDKANLEI |
| | | MTKRSNYTPRFLWQLKFECHFFNGTERVRLLERCIYNQEESVRFDSDVGEYRAVTELGRPDAEYWNSQKD |
| | | LLEQRRAAVDTYCRHNYGVGESFTVQRRGGSHHHHHHLE |
| 734 | S084 | IKEEHVIQQSEYYTNPDQSGEFQFDFDGDEIFHVDMAKKETVWRLEEFGRFASFEAQGALANIAVDKANLEI |
| | | MTKRSNYTGDTRPRFNWQLKFECHFFNGTERVRLLERCNYNQEESVRFDSDVGEYRAVTELGRPDAEYW |
| | | NSQKDLLEQRRAAVDTYCRHNYGVGESFTVQRRGGSHHHHHHLE |
| 735 | S085 | IKEEHVIIQAEFYLNPDQSGEFMFDFDGDEIFHVDMAKKETVWRLEEFGRFASFEAQGALANIAVDKANLEI |
| | | MTKYYNQSEAGSHLWQLKFECHFFNGTERVRLLERYIYNQEEYIRFDSDVGEWRAVTELGRITAEYWNSQ |
| | | KDLLEQRRAAVDTYCRHNYGVGESFTVQRRGGSHHHHHHLE |
| 736 | S086 | IKEEHVIIQAEFYLNPDQSGEFMFDFDGTEFFHVDMAKKETVWRLEEWGRFASFEAQGALANIAVDKANLE |
| | | IMTKRSNQSEAGSHLWQLKFECHFFNGTERVRLLERCIYNQEESVRFDSDVGEYRAVTELGRPDAEYWNS |
| | | QKDLLEQRRAAVDTYCRHNYGVYESWTVQRRGGSHHHHHHLE |
| 737 | S087 | IKEEHVIRQNETYLNPDQSGLFEFHLDGDPIFRVDMAKKETRWRLEEFGRFASFEAQGALANIAVDKANLEI |
| | | MTKRSNQSEAGSHSWQLIFVCEFHNGTERVRLSEHCYYNQELSVSFDSDVGEYRAVTELGRPDAEYWNSQ |
| | | KDLLEQRRAAVDTYCRHNYGVGESFTVQRRGGSHHHHHHLE |
| 738 | S088 | IKEEHVIRQNETYLNPDQSGEFEFDLDGDEIFRVDMAKKETRWRLEEFGRFASFEAQGALANIAVDKANLEI |
| | | MTKRSNQSEAGSHSWQLKFECEFHNGTERVRLSERCYYNQEESVRFDSDVGEYRAVTELGRPDAEYWNS |
| | | QKDLLEQRRAAVDTYCRHNYGVGESFTVQRRGGSHHHHHHLE |
| 739 | S089 | EPHSLRYNLTVLSWDGSVQSGFLTEVHLDGQEIFHVDMAKKETVWRLEEFGRFASFEAQGALANIAVDKA |
| | | NLEIMTKRDQKEGLHSLQEIRVCEIHEDNSTRSSQHFYYDGELFLSQNSDVGEYRAVTELGRPDAEYWNSQ |
| | | KDLLEQRRAAVDTYCRHNYGVGESFTVQRRGGSHHHHHHLE |
| 740 | S090 | IKEEHSIRQNEMYLNPDQSGLFEFHLDGQPIFRVDMQKKRTKWQLEEFGRFASFEAQGALANIAVDKANLE |
| | | IMTKRSNQSEAGSHSWQLIFVCEFHEGTESVRLSERCYYNQELSVSFNSDVGEYRAVTELGRPDAEYWNSQ |
| | | KDLLEQRRAAVDTYCRHNYGVGESFTVQRRGGSHHHHHHLE |
| 741 | S091 | IKEEHSIRQNEFYLNPDQSGEFEFDLDGDEIFRVDMQKKETKWRLEEFGRFASFEAQGALANIAVDKANLEI |
| | | MTKRSNQSEAGSHSWQLKFECEFHEGTERVRLSERCYYNQEESVRFDSDVGEYRAVTELGRPDAEYWNSQ |
| | | KDLLEQRRAAVDTYCRHNYGVGESFTVQRRGGSHHHHHHLE |
| 742 | S092 | EPHSLRYNLMVLSQDGSVQSGFLAEGHLDGQPFLRYDMAKKETVWRLEEFGRFASFEAQGALANIAVDKA |
| | | NLEIMTKRSDQKGGLHSLQEIRVCEIHEDSSTRGSRHFYYNGELFLSQNSDVGEYRAVTELGRPDAEYWNS |
| | | QKDLLEQRRAAVDTYCRHNYGVGESFTVQRRGGSHHHHHHLE |
| 743 | S093 | IKEEHVIIQAEFYLNPDQSGEFMFDFDGHEIFHVDMAKKETVWRLEEFGRFASFEAQGALANIAVDKANLEI |
| | | MTKRSNYTGDTRPRFLWQLKFECHFFNGTERVRLLERCIYNQEESVRFDSDVGEYRAVTELGRPDAEYWN |
| | | SQKDLLEQRRAAVDTYCRHNYGVGESFFVQRRGGSHHHHHHLE |
| 744 | S094 | GDTRPRFLWQLKFECHFFNGTERVRLLERCIYNQEESVRFDSDVGEYRAVTELGRPDAEYWNSQKDLLEQ |
| | | RRAAVDTYCRHNYGVGESFTVQRRGGYGGWSGGGGSGGGGIKEEHVIIQAEFYLNPDQSGEFMFDFDGDE |
| | | IFHVDMAKKETVWRLEEFGRFASFEAQGALANIAVDKANLEIMTKRSNYTPITNVPPEVTVLTNSPVELREP |
| | | NVLICFIDKFTPPVVNVTWLRNGKPVTTGVSETVFLPREDHLFRKFHYLPFLPSTEDVYDCRVEHWGLDEPL |
| | | LKHWEFDAPSGGSHHHHHHLE |
| 745 | S095 | VPTLKFVIFLDVKHFSPEDLTVKGRDIKEEHVIIQAEFYLNPDQSGEFMFDFDGDEIFHVDMAKKETVWRLE |
| | | EFGRFASFEAQGALANIAVDKANLEIMTKRSNYTPGGSMGDTRPRFLWQLKFECHFFNGTERVRLLERCIY |
| | | NQEESVRFDSDVGEYRAVTELGRPDAEYWNSQKDLLEQRRAAVDTYCRHNYGVGESFTVQGGSHHHHH |
| | | HLECCPGCCLE |
| 746 | S096 | SWDPFRDWIKEEHVIIQAEFYLNPDQSGEFMFDFDGDEIFHVDMAKKETVWRLEEFGRFASFEAQGALANI |
| | | AVDKANLEIMTKRSNYTPGGSMGDTRPRFLWQLKFECHFFNGTERVRLLERCIYNQEESVRFDSDVGEYR |
| | | AVTELGRPDAEYWNSQKDLLEQRRAAVDTYCRHNYGVGESFTVQGGSHHHHHHLE |
| 747 | S097 | SHSLKYFHTSVSRPGRGEPRFISVGYVDDTQFVRFDNDAASPRMVPRAPWMEQEGSEYWDRETRSARDTA |
| | | QIFRVNLRTLRGYYNQSEAGSHTLQWMHGCELGPDGRFLRGYEQFAYDGKDYLTLNEDLRSWTAVDTAA |
| | | QISEQKSNDASEAEHQRAYLEDTCVEWLHKYLEKGKETLLHLEPPKTHVTHHPISDHEATLRCWALGFYPA |
| | | EITLTWQQDGEGHTQDTELVETRPAGDGTFQKWAAVVVPSGEEQRYTCHVQHEGLPEPVTLRWKPASQPT |
| | | IPGGSHHHHHHLE |
| 748 | S098 | PENYLFQGRQECNGTQFLERYIYNREEFVRFDEFRAVGRPDEEYWNSQKDILEEERAVPDRMCRHNYELG |
| | | GPMTLQRRVQPRVNIKADHVSTYAAFVQRPFMFEFDEDEMFYVDLHLEEFGQAFSFEAQGGLANIAILNNN |
| | | LNTRSNHTQATNDPPHHHHHHLE |
| 749 | S099 | EDIVADHVASYGVNLYQSYGPSGQYTHEFDGDEQFYVDLGRKETVWCLPVLRQFRFDPQFALTNIAVLKH |
| | | NLNSLIKRSNSTAATNEVPRDSPEDFVYQFKGMCYFTNGTERVRLVSRSIYNREEIVRFDSDVGEFRAVTLL |
| | | GLPAAEYWNSQKDILERKRAAVDRVCRHNYQLELRTTLQRRVEPGGSMKAIFVLNAQHDEAVDANSALA |
| | | KAKVLANRELDKYGVSDYYKKLVNNAKTVEGVKDLQAQVVESAKKARGGSHHHHHHLE |
| 750 | S100 | HVLRYGYTGIFDDTSHMTLTVVGIFDGQHFFTYHVQSSDKASSRANGTISWMANVSAAYPTYLDGERAKG |
| | | DLIFNQTEQNLLELEIALGYRSQSVLTWTHECNTTENGSFVAGYEGFGWDGETLMELKDNLTLWTGPNYEI |
| | | SWLKQQKTYIDGKIKNISEGDTTIQRNYLKGNCTQWSVIYSGFQPPVTHPGGSHHHHHHLE |
| 751 | S101 | IKEEHVIIQAEFYLNPDQSGEFMFDFDGDEIFHVDMAKKETVWRLEEFGRFASFEAQGALANIAVDKANLEI |
| | | MTKRSNYTPMGDTRPRFLWQLKFESHFFNGTERVRLLERCIYNQEESVRFDSDVGEYRAVTELGRPDAEY |
| | | WNSQKDLLEQRRAAVDTYSRHNYGVGESFTVQGGSHHHHHHLE |
| 752 | S102 | IKEEHVIIQAEFYLNPDQSGEFMFDFDGDEIFHVDMAKKETVWRLEEFGRFASFEAQGALANIAVDKANLEI |
| | | MTKRSNYTPMGDTRPRFLWQLKFEKHFFNGTERVRLLERCIYNQEESVRFDSDVGEYRAVTELGRPDAEY |
| | | WNSQKDLLEQRRAAVDTYDRHNYGVGESFTVQGGSHHHHHHLE |
| 753 | S103 | DTRPRFLWQLKFEKHFFNGTERVRLLERCIYNQEESVRFDSDVGEYRAVTELGRPDAEYWNSQKDLLEQR |
| | | RAAVDTYDRHNYGVGESFTVQMIKEEHVIIQAEFYLNPDQSGEFMFDFDGDEIFHVDMAKKETVWRLEEF |
| | | GRFASFEAQGALANIAVDKANLEIMTKRSNYTPGGSHHHHHHLE |
| 754 | S104 | DTRPRFLWQLKFESHFFNGTERVRLLERCIYNQEESVRFDSDVGEYRAVTELGRPDAEYWNSQKDLLEQRR |
| | | AAVDTYSRHNYGVGESFTVQMIKEEHVIIQAEFYLNPDQSGEFMFDFDGDEIFHVDMAKKETVWRLEEFG |
| | | RFASFEAQGALANIAVDKANLEIMTKRSNYTPGGSHHHHHHLE |
| 755 | S105 | HVLRYGYTGIFDDTSHMTLTVVGIFDGQHFFTYHVQSSDKASSRANGLEEFGRFASFEAQGALANIAVDKA |
| | | NLEIMTKRSGYRSQSVLTWTHEKNTTENGSFVAGYEGFGWDGETLMELKDNLTLWTGPNYEIAVTELGRP |
| | | DAEYWNSQKDLLEQRRAAVDTYDRHNYGVGESFTVQGGSHHHHHHLE |
| 756 | S106 | HVLRYGYTGIFDDTSHMTLTVVGIFDGQHFFTYHVQSSDKASSRANGAVTELGRPDAEYWNSQKDLLEQR |
| | | RAAVDTYCRHNYGVGESGYRSQSVLTWTHECNTTENGSFVAGYEGFGWDGETLMELKDNLTLWTGPNY |
| | | EILEEFGRFASFEAQGALANIAVDKANLEIMTKRSFTVQGGSHHHHHHLE |
| 757 | S107 | GGGSGGGGSDTRPRFLWQLKFEKHFFNGTERVRLLERCIYNQEESVRFDSDVGEYRAVTELGRPDAEYWN |
| | | SQKDLLEQRRAAVDTYDRHNYGVGESFTVQMIKEEHVIIQAEFYLNPDQSGEFMFDFDGDEIFHVDMAKK |
| | | ETVWRLEEFGRFASFEAQGALANIAVDKANLEIMTKRSNYTPGGSHHHHHHLE |
| 758 | S108 | PENYLFQGRQEKNGTQFLERYIYNREEFVRFDEFRAVGRPDEEYWNSQKDILEEERAVPDRMDRHNYELG |
| | | GPMTLQRRVQPRVNIKADHVSTYAAFVQRPFMFEFDEDEMFYVDLHLEEFGQAFSFEAQGGLANIAILNNN |
| | | LNTRSNHTQATNDPPHHHHHHLE |
| 759 | S109 | SHSMRYFYTSVSRPGRGEPRFISVGYVDDTQFVRFDSDAASPREEPRAPWIEQEGPEYWDRNTQIYKAQAQ |
| | | TDRESLRNLRGYYNQSEAGSHTLQSMYGKDVGPDGRLLRGHDQYAYDGKDYIALNEDLRSWTAADTAA |
| | | QITQRKWEAAREAEQRRAYLEGEDVEWLRRYLENGKDKLERADPPGGSHHHHHHLE |
| 760 | S110 | GSSHHHHHHGGGGSGGGGSGDTRPRFLWQLKFEKHFFNGTERVRLLERCIYNQEESVRFDSDVGEYRAVT |
| | | ELGRPDAEYWNSQKDLLEQRRAAVDTYDRHNYGVGESFTVQRRVEPKVTVYPSKTQPLQHHNLLVDSVS |
| | | GFYPGSIEVRWFRNGQEEKAGVVSTGLIQNGDWTFQTLVMLETVPRSGEVYTKQVEHPSVTSPLTVEWRA |
| | | SGGGGSGGGGSGGGGIKEEHVIIQAEFYLNPDQSGEFMFDFDGDEIFHVDMAKKETVWRLEEFGRFASFEA |
| | | QGALANIAVDKANLEIMTKRSNYTPITNVPPEVTVLTNSPVELREPNVLIDFIDKFTPPVVNVTWLRNGKPV |
| | | TTGVSETVFLPREDHLFRKFHYLPFLPSTEDVYDKRVEHWGLDEPLLKHWEFDAPS |
| 761 | S111 | GKEEHVIIQAEFYLNPDQSGEFMFDFDGDEIFHVDMAKKETVWRLEEFEQEGPSYGDRNTQIYKAQAQTDR |
| | | ESLRNLRGYYNQSEAGSHFLWQLKFEKHFFNGTERVRLLERCIYDGKDYIALNEDLRSWTAADTAAQITQR |
| | | KWETQKEAEQRRAYLEGEDVEWLRRYLENGKDKLERADQRTPKIQVYSRHPAENGKSNFLNKYVSGFHP |
| | | SDIEVDLLKNGERIEKVEHSDLSFSKDWSFYLLYYTEFTPTEKDEYADRVNHVTLSQPKIVKWDRDMGGSH |
| | | HHHHH |
| 762 | S112 | GKEEHVIIQAEFYLNPDQSGEFMFDFDGDEIFHVDMAKKETVWRLEEFGRFASFEAQGALANIAVDKANLE |
| | | IMTKRSNYTPMGDTRPRFLWQLKFEKHFFNGTERVRLLERCIYNQEESVRFDSDVGEYRAVTELGRPDAEY |
| | | WNSQKDLLEQRRAAVDTYDRHNYGVGESFTVQQRTPKIQVYSRHPAENGKSNFLNKYVSGFHPSDIEVDL |
| | | LKNGERIEKVEHSDLSFSKDWSFYLLYYTEFTPTEKDEYADRVNHVTLSQPKIVKWDRDMGGSHHHHHHL |
| | | E |
| 763 | S113 | GSHSMRYYYTSVSRPGRGEPRFNSTGYVDDTQFVRFDSDAASPREEPRAPWIEQEGPEYWDRNTQIYKAQ |
| | | AQTDRESLRNLRGYYNQSEAGSHTLQSKYGCDVGPDGRLLRGHDQYAYDGKDYIALNEDLRSWTAADTA |
| | | AQITQRKWEAAREAEQRRAYLEGECVEWLRRYLENGKDKLERADQRTPKIQVYSRHPAENGKSNFLNCY |
| | | VSGFHPSDIEVDLLKNGERIEKVEHSDLSFSKDWSFYLLYYTEFTPTEKDEYACRVNHVTLSQPKIVKWDRD |
| | | MGGSHHHHHHLE |
| 764 | S114 | GSHSMRYFYTSVSRPGRGEPRFISVGYVDDTQFVRFDSDAASPREEPRAPWIEQEGPEYWDRNTQIYKAQA |
| | | QTDRESLRNLRGYYNQSEAGSHTLQSMYGCDVGPDGRLLRGHDQYAYDGKDYIALNEDLRSWTAADTA |
| | | AQITQRKWEAAREAEQRRAYLEGECVEWLRRYLENGKDKLERADPPQRTPKIQVYSRHPAENGKSNFLNC |
| | | YVSGFHPSDIEVDLLKNGERIEKVEHSDLSFSKDWSFYLLYYTEFTPTEKDEYACRVNHVTLSQPKIVKWDR |
| | | DMGGSHHHHHHLE |
| 765 | S115 | GSHSMRYFYTSVSRPGRGEPRFISVGYVDDTQFVRFDSDAASPREEPRAPWIEQEGPEAWDRNTQIYKAQA |
| | | QTDRESLRNLRGYYNQSEAGSHTLQSMYGCDVGPDGRLLRGHDQYAYDGKDSIALNEDLRSWTAADTAA |
| | | QITQRYWEAAREAEQRRAYLEGECVEHLRRVLENGKDKLERADQRTPKIQVYSRHPAENGKSNFLNCYVS |
| | | GFHPSDIEVDLLKNGERIEKVEHSDLSFSKDWSFYLLYYTEFTPTEKDEYACRVNHVTLSQPKIVKWDRDM |
| | | GGSHHHHHHLE |
| 766 | S116 | GDTRPRFLWQLKFECHFFNGTERVRLLERCIYNQEESVRFDSDVGEYRAVTELGRPDAEYWNSQKDLLEQ |
| | | RRAAVDTYCRHNYGVGESFTVQRRVEPKVTVYPSKTQPLQHHNLLVCSVSGFYPGSIEVRWFRNGQEEKA |
| | | GVVSTGLIQNGDWTFQTLVMLETVPRSGEVYTCQVEHPSVTSPLTVEWRASGGGGSGGGGSGGGGIKEEH |
| | | VIIQAEFYLNPDQSGEFMFDFDGDEIFHVDMAKKETVWRLEEFGRFASFEAQGALANIAVDKANLEIMTKR |
| | | SNYTPITNVPPEVTVLTNSPVELREPNVLICFIDKFTPPVVNVTWLRNGKPVTTGVSETVFLPREDHLFRKFH |
| | | YLPFLPSTEDVYDCRVEHWGLDEPLLKHWEFDAPSGGSHHHHHH |
| 767 | S117 | GDTRPRFLWQLKFECHFFNGTERVRLLERCIYNQEESVRFDSDVGEYRAVTELGRPDAEYWNSQKDLLEQ |
| | | RRAAVDTYCRHNYGVGESFTVQRRVEPKVTVYPSKTQPLQHHNLLVCSVSGFYPGSIEVRWFRNGQEEKA |
| | | GVVSTGLIQNGDWTFQTLVMLETVPRSGEVYTCQVEHPSVTSPLTVEWRASGGGGSGGGGSGGGGIKEEH |
| | | VIIQAEFYLNPDQSGEFMFDFDGDEIFHVDMAKKETVWRLEEFGRFASFEAQGALANIAVDKANLEIMTKR |
| | | SNYTPITNVPPEVTVLTNSPVELREPNVLICFIDKFTPPVVNVTWLRNGKPVTTGVSETVFLPREDHLFRKFH |
| | | YLPFLPSTEDVYDCRVEHWGLDEPLLKHWEFDAPSGGSHHHHHH |
| 768 | S118 | MVKYQKGDTRPRFLWQLKFECHFFNGTERVRLLERCIYNQEESVRFDSDVGEYRAVTELGRPDAEYWNSQ |
| | | KDLLEQRRAAVDTYCRHNYGVGESFTVQRRVEPKVTVYPSKTQPLQHHNLLVCSVSGFYPGSIEVRWFRN |
| | | GQEEKAGVVSTGLIQNGDWTFQTLVMLETVPRSGEVYTCQVEHPSVTSPLTVEWRASGGGGSGGGGSGG |
| | | GGIKEEHVIIQAEFYLNPDQSGEFMFDFDGDEIFHVDMAKKETVWRLEEFGRFASFEAQGALANIAVDKAN |
| | | LEIMTKRSNYTPITNVPPEVTVLTNSPVELREPNVLICFIDKFTPPVVNVTWLRNGKPVTTGVSETVFLPRED |
| | | HLFRKFHYLPFLPSTEDVYDCRVEHWGLDEPLLKHWEFDAPSGGSHHHHHH |
| 769 | S119 | MRRRIGDTRPRFLWQLKFECHFFNGTERVRLLERCIYNQEESVRFDSDVGEYRAVTELGRPDAEYWNSQK |
| | | DLLEQRRAAVDTYCRHNYGVGESFTVQRRVEPKVTVYPSKTQPLQHHNLLVCSVSGFYPGSIEVRWFRNG |
| | | QEEKAGVVSTGLIQNGDWTFQTLVMLETVPRSGEVYTCQVEHPSVTSPLTVEWRASGGGGSGGGGSGGG |
| | | GIKEEHVIIQAEFYLNPDQSGEFMFDFDGDEIFHVDMAKKETVWRLEEFGRFASFEAQGALANIAVDKANL |
| | | EIMTKRSNYTPITNVPPEVTVLTNSPVELREPNVLICFIDKFTPPVVNVTWLRNGKPVTTGVSETVFLPREDH |
| | | LFRKFHYLPFLPSTEDVYDCRVEHWGLDEPLLKHWEFDAPSGGSHHHHHH |
| 770 | S120 | MRIRRGDTRPRFLWQLKFECHFFNGTERVRLLERCIYNQEESVRFDSDVGEYRAVTELGRPDAEYWNSQK |
| | | DLLEQRRAAVDTYCRHNYGVGESFTVQRRVEPKVTVYPSKTQPLQHHNLLVCSVSGFYPGSIEVRWFRNG |
| | | QEEKAGVVSTGLIQNGDWTFQTLVMLETVPRSGEVYTCQVEHPSVTSPLTVEWRASGGGGSGGGGSGGG |
| | | GIKEEHVIIQAEFYLNPDQSGEFMFDFDGDEIFHVDMAKKETVWRLEEFGRFASFEAQGALANIAVDKANL |
| | | EIMTKRSNYTPITNVPPEVTVLTNSPVELREPNVLICFIDKFTPPVVNVTWLRNGKPVTTGVSETVFLPREDH |
| | | LFRKFHYLPFLPSTEDVYDCRVEHWGLDEPLLKHWEFDAPSGGSHHHHHH |
| 771 | S121 | MRRIRGDTRPRFLWQLKFECHFFNGTERVRLLERCIYNQEESVRFDSDVGEYRAVTELGRPDAEYWNSQK |
| | | DLLEQRRAAVDTYCRHNYGVGESFTVQRRVEPKVTVYPSKTQPLQHHNLLVCSVSGFYPGSIEVRWFRNG |
| | | QEEKAGVVSTGLIQNGDWTFQTLVMLETVPRSGEVYTCQVEHPSVTSPLTVEWRASGGGGSGGGGSGGG |
| | | GIKEEHVIIQAEFYLNPDQSGEFMFDFDGDEIFHVDMAKKETVWRLEEFGRFASFEAQGALANIAVDKANL |
| | | EIMTKRSNYTPITNVPPEVTVLTNSPVELREPNVLICFIDKFTPPVVNVTWLRNGKPVTTGVSETVFLPREDH |
| | | LFRKFHYLPFLPSTEDVYDCRVEHWGLDEPLLKHWEFDAPSGGSHHHHHH |
| 772 | S122 | MRIRRGDTRPRFLWQLKFECHFFNGTERVRLLERCIYNQEESVRFDSDVGEYRAVTELGRPDAEYWNSQK |
| | | DLLEQRRAAVDTYCRHNYGVGESFTVQRRVEPKVTVYPSKTQPLQHHNLLVCSVSGFYPGSIEVRWFRNG |
| | | QEEKAGVVSTGLIQNGDWTFQTLVMLETVPRSGEVYTCQVEHPSVTSPLTVEWRASGGGGSGGGGSGGG |
| | | GIKEEHVIIQAEFYLNPDQSGEFMFDFDGDEIFHVDMAKKETVWRLEEFGRFASFEAQGALANIAVDKANL |
| | | EIMTKRSNYTPITNVPPEVTVLTNSPVELREPNVLICFIDKFTPPVVNVTWLRNGKPVTTGVSETVFLPREDH |
| | | LFRKFHYLPFLPSTEDVYDCRVEHWGLDEPLLKHWEFDAPSGGSHHHHHHLE |
| 773 | S123 | MIRPRGDTRPRFLWQLKFECHFFNGTERVRLLERCIYNQEESVRFDSDVGEYRAVTELGRPDAEYWNSQKD |
| | | LLEQRRAAVDTYCRHNYGVGESFTVQRRVEPKVTVYPSKTQPLQHHNLLVCSVSGFYPGSIEVRWFRNGQ |
| | | EEKAGVVSTGLIQNGDWTFQTLVMLETVPRSGEVYTCQVEHPSVTSPLTVEWRASGGGGSGGGGSGGGGI |
| | | KEEHVIIQAEFYLNPDQSGEFMFDFDGDEIFHVDMAKKETVWRLEEFGRFASFEAQGALANIAVDKANLEI |
| | | MTKRSNYTPITNVPPEVTVLTNSPVELREPNVLICFIDKFTPPVVNVTWLRNGKPVTTGVSETVFLPREDHLF |
| | | RKFHYLPFLPSTEDVYDCRVEHWGLDEPLLKHWEFDAPSGGSHHHHHH |
| 774 | S124 | GSHSMRYFYTSVSRPGRGEPRFISVGYVDDTQFVRFDSDAASPREEPRAPWIEQEGPEYWDRNTQIYKAQA |
| | | QTDRESLRNLRGYYNQSEAGSHTLQSMYGCDVGPDGRLLRGHDQYAYDGKDYIALNEDLRSWTAADTA |
| | | AQITQRKWEAAREAEQRRAYLEGECVEWLRRYLENGKDKLERADPPKTHVTHHPISDHEATLRCWALGF |
| | | YPAEITLTWQRDGEDQTQDTELVETRPAGDRTFQKWAAVVVPSGEEQRYTCHVQHEGLPKPLTLRWESGG |
| | | GGSGGGGSGGGGSGGGGIQRTPKIQVYSRHPAENGKSNFLNCYVSGFHPSDIEVDLLKNGERIEKVEHSDLS |
| | | FSKDWSFYLLYYTEFTPTEKDEYACRVNHVTLSQPKIVKWDRDMGGSHHHHHH |
| 775 | S125 | GSHSMRYFYTSVSRPGRGEPRFISVGYVDDTQFVRFDSDAASPREEPRAPWIEQEGPEYWDRNTQIYKAQA |
| | | QTDRESLRNLRGYYNQSEAGSHTLQSMYGCDVGPDGRLLRGHDQYAYDGKDYIALNEDLRSWTAADTA |
| | | AQITQRKWEAAREAEQRRAYLEGECVEWLRRYLENGKDKLERADPPKTHVTHHPISDHEATLRCWALGF |
| | | YPAEITLTWQRDGEDQTQDTELVETRPAGDRTFQKWAAVVVPSGEEQRYTCHVQHEGLPKPLTLRWESGG |
| | | GGSGGGGSGGGGSGGGGIQRTPKIQVYSRHPAENGKSNFLNCYVSGFHPSDIEVDLLKNGERIEKVEHSDLS |
| | | FSKDWSFYLLYYTEFTPTEKDEYACRVNHVTLSQPKIVKWDRDMGGSHHHHHH |
| 776 | S126 | MVKYQKGSHSMRYFYTSVSRPGRGEPRFISVGYVDDTQFVRFDSDAASPREEPRAPWIEQEGPEYWDRNT |
| | | QIYKAQAQTDRESLRNLRGYYNQSEAGSHTLQSMYGCDVGPDGRLLRGHDQYAYDGKDYIALNEDLRSW |
| | | TAADTAAQITQRKWEAAREAEQRRAYLEGECVEWLRRYLENGKDKLERADPPKTHVTHHPISDHEATLRC |
| | | WALGFYPAEITLTWQRDGEDQTQDTELVETRPAGDRTFQKWAAVVVPSGEEQRYTCHVQHEGLPKPLTLR |
| | | WESGGGGSGGGGSGGGGSGGGGIQRTPKIQVYSRHPAENGKSNFLNCYVSGFHPSDIEVDLLKNGERIEKV |
| | | EHSDLSFSKDWSFYLLYYTEFTPTEKDEYACRVNHVTLSQPKIVKWDRDMGGSHHHHHH |
| 777 | S127 | MRRRIGSHSMRYFYTSVSRPGRGEPRFISVGYVDDTQFVRFDSDAASPREEPRAPWIEQEGPEYWDRNTQIY |
| | | KAQAQTDRESLRNLRGYYNQSEAGSHTLQSMYGCDVGPDGRLLRGHDQYAYDGKDYIALNEDLRSWTA |
| | | ADTAAQITQRKWEAAREAEQRRAYLEGECVEWLRRYLENGKDKLERADPPKTHVTHHPISDHEATLRCW |
| | | ALGFYPAEITLTWQRDGEDQTQDTELVETRPAGDRTFQKWAAVVVPSGEEQRYTCHVQHEGLPKPLTLRW |
| | | ESGGGGSGGGGSGGGGSGGGGIQRTPKIQVYSRHPAENGKSNFLNCYVSGFHPSDIEVDLLKNGERIEKVE |
| | | HSDLSFSKDWSFYLLYYTEFTPTEKDEYACRVNHVTLSQPKIVKWDRDMGGSHHHHHH |
| 778 | S128 | MRIRRGSHSMRYFYTSVSRPGRGEPRFISVGYVDDTQFVRFDSDAASPREEPRAPWIEQEGPEYWDRNTQIY |
| | | KAQAQTDRESLRNLRGYYNQSEAGSHTLQSMYGCDVGPDGRLLRGHDQYAYDGKDYIALNEDLRSWTA |
| | | ADTAAQITQRKWEAAREAEQRRAYLEGECVEWLRRYLENGKDKLERADPPKTHVTHHPISDHEATLRCW |
| | | ALGFYPAEITLTWQRDGEDQTQDTELVETRPAGDRTFQKWAAVVVPSGEEQRYTCHVQHEGLPKPLTLRW |
| | | ESGGGGSGGGGSGGGGSGGGGIQRTPKIQVYSRHPAENGKSNFLNCYVSGFHPSDIEVDLLKNGERIEKVE |
| | | HSDLSFSKDWSFYLLYYTEFTPTEKDEYACRVNHVTLSQPKIVKWDRDMGGSHHHHHH |
| 779 | S129 | MRRIRGSHSMRYFYTSVSRPGRGEPRFISVGYVDDTQFVRFDSDAASPREEPRAPWIEQEGPEYWDRNTQIY |
| | | KAQAQTDRESLRNLRGYYNQSEAGSHTLQSMYGCDVGPDGRLLRGHDQYAYDGKDYIALNEDLRSWTA |
| | | ADTAAQITQRKWEAAREAEQRRAYLEGECVEWLRRYLENGKDKLERADPPKTHVTHHPISDHEATLRCW |
| | | ALGFYPAEITLTWQRDGEDQTQDTELVETRPAGDRTFQKWAAVVVPSGEEQRYTCHVQHEGLPKPLTLRW |
| | | ESGGGGSGGGGSGGGGSGGGGIQRTPKIQVYSRHPAENGKSNFLNCYVSGFHPSDIEVDLLKNGERIEKVE |
| | | HSDLSFSKDWSFYLLYYTEFTPTEKDEYACRVNHVTLSQPKIVKWDRDMGGSHHHHHH |
| 780 | S130 | MRIRRGSHSMRYFYTSVSRPGRGEPRFISVGYVDDTQFVRFDSDAASPREEPRAPWIEQEGPEYWDRNTQIY |
| | | KAQAQTDRESLRNLRGYYNQSEAGSHTLQSMYGCDVGPDGRLLRGHDQYAYDGKDYIALNEDLRSWTA |
| | | ADTAAQITQRKWEAAREAEQRRAYLEGECVEWLRRYLENGKDKLERADPPKTHVTHHPISDHEATLRCW |
| | | ALGFYPAEITLTWQRDGEDQTQDTELVETRPAGDRTFQKWAAVVVPSGEEQRYTCHVQHEGLPKPLTLRW |
| | | ESGGGGSGGGGSGGGGSGGGGIQRTPKIQVYSRHPAENGKSNFLNCYVSGFHPSDIEVDLLKNGERIEKVE |
| | | HSDLSFSKDWSFYLLYYTEFTPTEKDEYACRVNHVTLSQPKIVKWDRDMGGSHHHHHHLE |
| 781 | S131 | MIRPRGSHSMRYFYTSVSRPGRGEPRFISVGYVDDTQFVRFDSDAASPREEPRAPWIEQEGPEYWDRNTQIY |
| | | KAQAQTDRESLRNLRGYYNQSEAGSHTLQSMYGCDVGPDGRLLRGHDQYAYDGKDYIALNEDLRSWTA |
| | | ADTAAQITQRKWEAAREAEQRRAYLEGECVEWLRRYLENGKDKLERADPPKTHVTHHPISDHEATLRCW |
| | | ALGFYPAEITLTWQRDGEDQTQDTELVETRPAGDRTFQKWAAVVVPSGEEQRYTCHVQHEGLPKPLTLRW |
| | | ESGGGGSGGGGSGGGGSGGGGIQRTPKIQVYSRHPAENGKSNFLNCYVSGFHPSDIEVDLLKNGERIEKVE |
| | | HSDLSFSKDWSFYLLYYTEFTPTEKDEYACRVNHVTLSQPKIVKWDRDMGGSHHHHHH |
| 782 | S132 | MGDTRPRFLWQPKRECHFFNGTERVRFLDRYFYNQEESVRFDSDVGEFRAVTELGRPDAEYWNSQKDILE |
| | | QARAAVDTYCRHNYGVVESFTVQRRVIKEEHVIIQAEFYLNPDQSGEFMFDFDGDEIFHVDMAKKETVWR |
| | | LEEFGRFASFEAQGALANIAVDKANLEIMTKRSNYTPITN |
| 783 | S133 | MGDTRPRFLWQPKRECHFFNGTERVRFLDRYFYNQEESVRFDSDVGEFRAVTELGRPDAEYWNSQKDILE |
| | | QARAAVDTYCRHNYGVVESFTVQRRVIKEEHVIIQAEFYLNPDQSGEFMFDFDGDEIFHVDMAKKETVWR |
| | | LEEFGRFASFEAQGALANIAVDKANLEIMTKRSNYTPITNGGSHHHHHH |
| 784 | S134 | MVKYHHHHHHGGSENLYFQGMGDTRPRFLWQPKRECHFFNGTERVRFLDRYFYNQEESVRFDSDVGEFR |
| | | AVTELGRPDAEYWNSQKDILEQARAAVDTYCRHNYGVVESFTVQRRVIKEEHVIIQAEFYLNPDQSGEFMF |
| | | DFDGDEIFHVDMAKKETVWRLEEFGRFASFEAQGALANIAVDKANLEIMTKRSNYTPITN |
| 785 | S135 | MGDTPENYLFQGRQECYAFNGTQRFLERYIYNREEFVRFDSDVGEFRAVTELGRPDEEYWNSQKDILEEER |
| | | AVPDRMCRHNYELGGPMTLQRRVIKADHVSTYAAFVQTHRPTGEFMFEFDEDEMFYVDLDKKETVWHLE |
| | | EFGQAFSFEAQGGLANIAILNNNLNTLIQRSNHTQATN |
| 786 | S136 | MVKYHHHHHHGGSMGDTPENYLFQGRQECYAFNGTQRFLERYIYNREEFVRFDSDVGEFRAVTELGRPDE |
| | | EYWNSQKDILEEERAVPDRMCRHNYELGGPMTLQRRVIKADHVSTYAAFVQTHRPTGEFMFEFDEDEMFY |
| | | VDLDKKETVWHLEEFGQAFSFEAQGGLANIAILNNNLNTLIQRSNHTQATN |
| 787 | S137 | MGDTPENYLFQGRQECYAFNGTQRFLERYIYNREEFARFDSDVGEFRAVTELGRPAAEYWNSQKDILEEKR |
| | | AVPDRMCRHNYELGGPMTLQRRVIKADHVSTYAAFVQTHRPTGEFMFEFDEDEMFYVDLDKKETVWHLE |
| | | EFGQAFSFEAQGGLANIAILNNNLNTLIQRSNHTQATN |
| 788 | S138 | MVKYHHHHHHGGSGDTPENYLFQGRQECYAFNGTQRFLERYIYNREEFARFDSDVGEFRAVTELGRPAAE |
| | | YWNSQKDILEEKRAVPDRMCRHNYELGGPMTLQRRVIKADHVSTYAAFVQTHRPTGEFMFEFDEDEMFY |
| | | VDLDKKETVWHLEEFGQAFSFEAQGGLANIAILNNNLNTLIQRSNHTQATN |
| 789 | S139 | MIKEEHVIIQAEFYLNPDQSGEFMFDFDGDEIFHVDMAKKETVWRLEEFGRFASFEAQGALANIAVDKANL |
| | | EIMTKRSNYTPTINGDTRPRFLWQLKFECHFFNGTERVRLLERCIYNQEESVRFDSDVGEYRAVTELGRPDA |
| | | EYWNSQKDLLEQRRAAVDTYCRHNYGVGESFTVQRRV |
| 790 | S140 | MVKYHHHHHHGGSIKEEHVIIQAEFYLNPDQSGEFMFDFDGDEIFHVDMAKKETVWRLEEFGRFASFEAQ |
| | | GALANIAVDKANLEIMTKRSNYTPTINGDTRPRFLWQLKFECHFFNGTERVRLLERCIYNQEESVRFDSDVG |
| | | EYRAVTELGRPDAEYWNSQKDLLEQRRAAVDTYCRHNYGVGESFTVQRRV |
| 791 | S141 | MGDTRPRFLWQLKFECHFFNGTERVRLLERCIYNQEESVRFDSDVGEYRAVTELGRPDAEYWNSQKDLLE |
| | | QRRAAVDTYCRHNYGVGESFTVQRRVIKEEHVIIQAEFYLNPDQSGEFMFDFDGDEIFHVDMAKKETVWR |
| | | LEEFGRFASFEAQGALANIAVDKANLEIMTKRSNYTPTIN |
| 792 | S142 | MVKYHHHHHHGGSGDTRPRFLWQLKFECHFFNGTERVRLLERCIYNQEESVRFDSDVGEYRAVTELGRPD |
| | | AEYWNSQKDLLEQRRAAVDTYCRHNYGVGESFTVQRRVIKEEHVIIQAEFYLNPDQSGEFMFDFDGDEIFH |
| | | VDMAKKETVWRLEEFGRFASFEAQGALANIAVDKANLEIMTKRSNYTPTIN |
| 793 | S143 | MGDTRPRFLWQLKFECHFFNGTERVRLLERCIYNQEESVRFDSDVGEYRAVTELGRPDAEYWNSQKDLLE |
| | | QRRAAVDTYCRHNYGVGESFTVQRRVIKEEHVIIQAEFYLNPDQSGEFMFDFDGDEIFHVDMAKKETVWR |
| | | LEEFGRFASFEAQGALANIAVDKANLEIMTKRSNYTPTINGGSHHHHHH |
| 794 | S144 | MRDSPEDFVYQFKGMCYFTNGTERVRLVSRSIYNREEIVRFDSDVGEFRAVTLLGLPAAEYWNSQKDILER |
| | | KRAAVDRVCRHNYQLELRTTLQRRVVEDIVADHVASYGVNLYQSYGPSGQYTHEFDGDEQFYVDLGRKE |
| | | TVWCLPVLRQFRFDPQFALTNIAVLKHNLNSLIKRSNSTAATN |
| 795 | S145 | MVKYHHHHHHGGSRDSPEDFVYQFKGMCYFTNGTERVRLVSRSIYNREEIVRFDSDVGEFRAVTLLGLPA |
| | | AEYWNSQKDILERKRAAVDRVCRHNYQLELRTTLQRRVVEDIVADHVASYGVNLYQSYGPSGQYTHEFD |
| | | GDEQFYVDLGRKETVWCLPVLRQFRFDPQFALTNIAVLKHNLNSLIKRSNSTAATN |
| 796 | S146 | MGDTRPRFLWQLKFECHFFNGTERVRLLERCIYNQEESVRFDSDVGEYRAVTELGRPDAEYWNSQKDLLE |
| | | QRRAAVDTYCRHNYGVGESFTVQRRVEDIVADHVASYGVNLYQSYGPSGQYTHEFDGDEQFYVDLGRKE |
| | | TVWCLPVLRQFRFDPQFALTNIAVLKHNLNSLIKRSNSTAATN |
| 797 | S147 | MVKYHHHHHHGGSGDTRPRFLWQLKFECHFFNGTERVRLLERCIYNQEESVRFDSDVGEYRAVTELGRPD |
| | | AEYWNSQKDLLEQRRAAVDTYCRHNYGVGESFTVQRRVEDIVADHVASYGVNLYQSYGPSGQYTHEFDG |
| | | DEQFYVDLGRKETVWCLPVLRQFRFDPQFALTNIAVLKHNLNSLIKRSNSTAATN |
| 798 | S148 | GIKEEHVIIQAEFYLNPDQSGEFMFDFDGDEIFHVDMAKKETVWRLEEFGRFASFEAQGALANIAVDKANL |
| | | EIMTKRSNYTPITNGDTQPRFLEQAKCECHFLNGTERVWNLIRYIYNQEEYARYNSDLGEYQAVTELGRPD |
| | | AEYWNSQKDLLERRRAEVDTYCRYNYGVVESFTVQRR |
| 799 | S149 | GDTQPRFLEQAKCECHFLNGTERVWNLIRYIYNQEEYARYNSDLGEYQAVTELGRPDAEYWNSQKDLLER |
| | | RRAEVDTYCRYNYGVVESFTVQRRIKEEHVIIQAEFYLNPDQSGEFMFDFDGDEIFHVDMAKKETVWRLEE |
| | | FGRFASFEAQGALANIAVDKANLEIMTKRSNYTPITN |
| 800 | S150 | GRDPPEDFVLQFKAMCYFTNGTERVRYVTRYIYNREEDVRFDSDVGVYRAVTPQGRPDAEYWNSQKDILE |
| | | RTRAELDTVCRHNYEVAFRGILQRREDIVADHVASYGVNLYQSYGPSGQFTHEFDGDEEFYVDLERKETV |
| | | WKLPLFHRLRFDPQFALTNIAVLKHNLNILIKRSNSTAATN |
| 801 | S151 | GEDIVADHVASYGVNLYQSYGPSGQFTHEFDGDEEFYVDLERKETVWKLPLFHRLRFDPQFALTNIAVLKH |
| | | NLNILIKRSNSTAATNRDPPEDFVLQFKAMCYFTNGTERVRYVTRYIYNREEDVRFDSDVGVYRAVTPQGR |
| | | PDAEYWNSQKDILERTRAELDTVCRHNYEVAFRGILQRR |
| 802 | S152 | GEDIVADHVASYGVNLYQSYGPSGQYTHEFDGDEQFYVDLGRKETVWCLPVLRQFRFDPQFALTNIAVLK |
| | | HNLNSLIKRSNSTAATNRDSPEDFVYQFKGMCYFTNGTERVRLVSRSIYNREEIVRFDSDVGEFRAVTLLGL |
| | | PAAEYWNSQKDILERKRAAVDRVCRHNYQLELRTTLQRR |
| 803 | S153 | GDTRPRFLWQLIFVCEFHEGTESVRLSERCYYNQELSVSFNSDVGEYRAVTELGRPDAEYWNSQKDLLEQR |
| | | RAAVDTYCRHNYGVGESFTVQRRSGGGKEEHSIRQNEMYLNPDQSGLFEFHLDGQPIFRVDMQKKRTKW |
| | | QLEEFGRFASFEAQGALANIAVDKANLEIMTKRSNYT |
| 804 | S154 | GIKEEHSIRQNEMYLNPDQSGLFEFHLDGQPIFRVDMQKKRTKWQLEEFGRFASFEAQGALANIAVDKANL |
| | | EIMTKRSNQSEAGSHSWQLIFVCEFHEGTESVRLSERCYYNQELSVSFNSDVGEYRAVTELGRPDAEYWNS |
| | | QKDLLEQRRAAVDTYCRHNYGVGESFTVQRR |
| 805 | S155 | GSHSMRYYYTSVSRPGRGEPRFNSTGYVDDTQFVRFDSDAASPRYEPRAPWIEQETPEYWDRNTQDYKAQ |
| | | AQTDREYLRNLRWYYNQSEAGSHTLQSKYGCDVGPDGRLLRGHDQYAYDGKDYIALNEDLRSWTAADT |
| | | AAQYTQRLPEAARLAEQRRATLEGLCVEWLRRSLENGKDKLER |
| 806 | S156 | GSHSMRYYDTSVSRPGRGEPRFNSTGYVDDTQFVRFDSDAASPREEPRAPWIEQEGPEYWDRNTQDYKAQ |
| | | AQDDRMYLRNLRGWYNQSEAGSHTLQSKDGCDVGPDGRLLRGHDQLAYDGKDYIALNEDLRSWTAADT |
| | | AAQILQRLWEAARLAELIRAYLEGERVEWLRRDLENGKDKLER |
| 807 | S157 | GSHSMRYYDTSVSRPGRGEPRFNSTGYVDDTQFVRFDSDAASPRMEPRAPWIEQEGPEPVDRNTQIYKAQA |
| | | QTDRESLRNLRGYYNQSEAGSHTLQSKAGCDVGPDGRLLRGHDQWAYDGKDYIALNEDLRSWTAADTA |
| | | AQITQRHWEAAREAEQRRAYLEGECVETLRRQLENGKDKLER |
| 808 | S158 | GSHSMRYYDTSVSRPGRGEPRFNSTGYVDDTQFVRFDSDAASPRMEPRAPWIEQEGPEPVDRNTQIYKAQA |
| | | QTDRESLRNLRGYYNQSEAGSHTLQSKAGCDVGPDGRLLRGHDQWAYDGKDYIALNEDLRSWTAADTA |
| | | AQITQRHWEAAREAEQRRAYLEGECVETLRRQLENGKDKLER |
| 809 | S159 | GSHSMRYYDTSVSRPGRGEPRFNSTGYVDDTQFVRFDSDAASPRMEPRAPWIEQEGPEPVDRNTQIYKAQA |
| | | QTDRESLRNLRGYYNQSEAGSHTLQSKAGCDVGPDGRLLRGHDQWAYDGKDYIALNEDLRSWTAADTA |
| | | AQITQRHWEAAREAEQRRAYLEGECVETLRRQLENGKDKLER |
| 810 | S160 | GSHSMRYFYTSVSRPGRGEPRFISVGYVDDTQFVRFDSDAASPREEPRAPWIEQEGPEGWDRNTQGYKAQA |
| | | QTDRESLRNMRGYYNQSEAGSHTLQSMYGCDVGPDGRLLRGHDQYAYDGKDYIALNEDLRSWTAADTA |
| | | AQIDQRKWEAAREAEQRRAYLEGECVEWLRRYLENGKDKLERAD |
| 811 | S161 | GIKEEHSIRQNEKYLNPDQSGNFEFHLDGQPIFRVDMQKKRTKWQLEEFGRFASFEAQGALANIAVDKANL |
| | | EIMTKRSNQSEAGSHSWQLNFVCEFHEGTESVRLSERCYYNQENSVSFNSDVGEYRAVTELGRPDAEYWN |
| | | SQKDLLEQRRAAVDTYCRHNYGVGESFTVQRR |
| 812 | S162 | GIKEEHSIRQNEKYNNPDQSGNFEFHLDGQPIFRVDMQKKRTKWQLEEFGRFASFEAQGALANIAVDKANL |
| | | EIMTKRSNQSEAGSHSWQLNFVCEFHEGTESVRLSERCYYNQENSVSFNSDVGEYRAVTELGRPDAEYWN |
| | | SQKDLLEQRRAAVDTYCRHNYGVGESFTVQRR |
| 813 | S163 | GIKEEHSIRQNEKYNNPDQSGNFEFHLDGQPIFRVDMQKKRTKWQLEEFGRFASFEAQGALANIAVDKANL |
| | | EIMTKRSNQSEAGSHSWQLNFTCEFHEGTESVRLSERCYYNQENSVSFNSDVGEYRATTELGRPDAEYWNS |
| | | QKDLLEQRRAAVDTYCRHNYGVGESFTVQRR |
| 814 | S164 | GIKEEHSIRQNEKYNNPDQSGNFEFHLDGQPIFRVDMQKKRTKWQLEEFGRFASFEAQGALANIAVFKINLE |
| | | IMTKRSNQSEAGSHSWQLNFTCEFHEGTESVRLSERSYYNQENFVSFNSDVGEYRATTELGRPMAEYVNSQ |
| | | KDTLEQRRAAVDTYCRHNYGVGEYFTVQRR |
| 815 | S165 | GIKEEHSIRYNEKYNNPDQSGNFEFHLDGQPIWRVDMQKKRTKWQLEEFGRFASFEAQQALANIADDKYN |
| | | LEIMTSRSNQSEAGSHSWQLNFTCEFHEGTESVRLSNRCYYNQENSVSFNSDVGEYRATTELGRPDAEYWN |
| | | SQKDSLEQRRAIVDTYCRHNYGVGESFTVQRR |
| 816 | S166 | GIKEEHSIRQNEKYNNPDQSGNFEFHLDGQPISRVDMQKKRTKWQLEEFGRYASFEAQEALAFIAVDKANL |
| | | EIMTKRSNQSEAGSHSWQLNFTCEFHEGTESVRLSERCYYNQENSVSFNSDVGEYRATTELGRPIAEYGNS |
| | | QKDLLEKRRAIVDTSCRHNYGVGEEFTVQRR |
| 817 | S167 | GIKEEHSIRQNVKYNNPDQSGNFEFHLDGQPIFRVDMQKKRTKWQLEEFGREPSFEAQGYLANIAVDKYNL |
| | | EIMTKNSNQSEAGSHSWQLNMTCEFHEGTESVRLSERDYYNQENSVSFNSDVGEYRATTELGRPDAEYRN |
| | | SQKDLLEQRRAVVDTVCRHNYGVGESFTVNRR |
| 818 | S168 | GIKEEHSYRTNEKYNNPDQSGNFEFHLDGQPIFRVDMQKKRTKWQLEEFGREASFEAQPYLANIAVDKAN |
| | | LEIMTKRSNQSEAGSHSWQINFTCEFHEGTESVRLSERCYYNQENSVSFNSDVGEYRATTELGRPDANYQN |
| | | SQKDLLEQRRAWVDTYCRHNYGVGELFTVQRR |
| 819 | S169 | GDTRPRFLWQLKFECHFYNGTERVRLNERCNFYNQEESVRFDSDVGEYRAVTELGRPDAEYWNSQKDLLE |
| | | QRRAAVDTYCRHNYGVGESFTVQRRSGGGIKEEHVINQAEYYLNPDQSGEFKFDFDGDEIFHVDMAKKET |
| | | VWRLEEFGRFASFEAQGALANIAVDKANLEIMTKRSNYT |
| 820 | S170 | GDTRPRFLWQLKFECHFYNGTERVRLNERCNYNQEESVRFDSDVGEYRAVTELGRPVAEYVNSQKDLLEQ |
| | | HRAAVDTECRHNYGVGESFTVQRRSGGGIKEEHVINQAEYYLNPDQSGEFKFDFDGDEIFHVDMAKKETV |
| | | WRLEEFGRFASTEAQGALYNIAVSKANLEIMTKRSNYT |
| 821 | S171 | GDTRPRFLWQLKWECHFYNGTERVRLNERCNYNQEESVRFDSDVGEYRAVTELGRPDAEFSNSQKDNLE |
| | | QRRAAVDTLCRHNYGVGESFTVQRRSGGGIKEEHVINQAEYYLNPDQSGEFKFDFDGDEIFHVDMAKKET |
| | | VWRLEEFGRFAIIEAYGALANIAVDKANLEIMTKQSNYT |
| 822 | S172 | GDTRPRFLWQLKFECHFYNGTERVRLNERCNYNQEESVRFDSDVGEYRAVTELGRPDAEDSNSQKDLLET |
| | | RRAGVDTYCRHNYGVGESFTVQRRSGGGIKEEHVINQAEYYLNPDQSGEFKFDFDGDEWFHVDMAKKET |
| | | VWRLEEFGRFASNEAQGALANIAVDVANLEIMLKRSNYT |
| 823 | S173 | GDTRPRFLWQLKFECHFYNGTERVRLNERNNYNQEESVRFDSDVGEYRAVTELGRPDAEYTNSQKDALEQ |
| | | RRAWVDTYCRMNEGVGESFTVQRRSGGGIKEEHVINQAEYYLNPDQSGEFKTDFDGDEIFHVDMAKKETV |
| | | WRLEEFGRFASFEAQGALENIAVDKANLEIMTKRSNYT |
| 824 | S174 | GDTRPRFLWQLKFECHFYNGTERVRLNERSNYNQEESVRFDSDVGEYRAVTELGRPDAEYWNSGKDQLE |
| | | QRRAAVDTICRFNYGVGESFTVQRRSGGGIKEEHVINVAEYYLNPDQSGEFKFDFDGDEIFHVDMAKKETV |
| | | WRLEEFGRENSFEAEGALANIAVDKANLEIMTKRSNYT |
| 825 | S175 | MIKEEHSIRQNEKYLNPDQSGNFEFHLDGQPIFRVDMQKKRTKWQLEEFGRFASFPAQGALANIAVDKANL |
| | | EIMTKRSNQSEAGSHSWQLNFVCEFHEGTESVRLSERCYYNQELSVSFNSDVGEYRAVTELGRPDAEYWN |
| | | SQKDLLEQRRAAVDHVCRHNYGVGESFTVQRRGGSHHHHHHLE |
| 826 | S176 | MIKEEHSIRWNEKYLNPDQSGNFEFHLDGQPIFRVDMQKKRTKWQLEEFGRFASFEAQFPLAIIAVKKANL |
| | | EKMTKHSNQSEAGSHSWQHNFVCEFHEGTESVRLSERCYYNQELSVSFNSDVGEGRAVTELGRPDAEYHN |
| | | SQKDLLEQRRAAVDHYCRYNYGVGESFTVQRRGGSHHHHHHLE |
| 827 | S177 | MIKEEHVINQAEYYLNPDQSGEFKFDFDGDEIFHVDMAKKETVWRLEEFGRFASFEAQGALAHIAVDKAN |
| | | LEIMTKRSNYTPMGDTRPRFLWQLKYECHFFNGTERVRLNERCIYNQEESVRFDSDVGEYRAVTELGRPDA |
| | | EQWNSQKDHLEQRRAAVDTKCRHNYGVGESFTVQGGSHHHHHHLE |
| 828 | S178 | MIKEEHVINQAEYYLNPDQSGEFKFDFDGDEIFHVDMAKKETVWRLEEFGRFASMEAQGWLARIAVDKAN |
| | | LEIMTVPSNYTPMGDTRPRFLWQLKFECHFFNGTERVRLNERDIYNQEESVRFDSDVGENRAVTELGRPTA |
| | | EDKNSQKDLLEQRRAAVDTYCRDNYGVGESFTVQGGSHHHHHHLE |
| 829 | S179 | MIKEEHVINYAEYYLNPDQSGEFKFDFDGDEIFHVDMAKKETVWRLEEFGRFASFEAQGPLANIAVDKANL |
| | | ESMTKDSNYTPMGDTRPRFLYQLKFECHFFNGTERVRLNERCIYNQEESVRFDSDVGEYRAVTELGRKDAE |
| | | HHNSQKDLLEQRRAAVDKACRHAYGSGESFTVQGGSHHHHHHLE |
| 830 | S180 | MKEEHVIIQAEFYLNPDQSGEFMFDFDGDEIFHVDMAKKETVWRLEEFGRFASFEAQGALANIAVDKANLE |
| | | IMTKRSNYTPITNTRPRFLWQPKRECHFFNGTERVRFLDRYFYNQEESVRFDSDVGEFRAVTELGRPDAEY |
| | | WNSQKDILEQARAAVDTYCRHNYGVVESFTVQRRVQGGSHHHHHHLE |
| 831 | S181 | MSHSLKYFHTSVSRPGRGEPRFISVGYVDDTQFVRFDNDAASPRMVPRAPWMEQEGSEYWDRETRSARDT |
| | | AQIFRVNLRTLRGYYNQSEAGSHTLQWMHGCELGPDGRFLRGYEQFAYDGKDYLTLNEDLRSWTAVDTA |
| | | AQISEQKSNDASEAEHQRAYLEDTCVEWLHKYLEKGKETLLHLEGGSHHHHHHLE |
| 832 | S182 | MSHSMRYFSAAVSRPGRGEPRFIAMGYVDDTQFVRFDSDSACPRMEPRAPWVEQEGPEYWEEETRNTKA |
| | | HAQTDRMNLQTLRGYYNQSEASSHTLQWMIGCDLGSDGRLLRGYEQYAYDGKDYLALNEDLRSWTAAD |
| | | TAAQISKRKCEAANVAEQRRAYLEGTCVEWLHRYLENGKEMLQRADGGSHHHHHHLE |
| 833 | S183 | MGSHSLRYFSTAVSRPGRGEPRYIAVEYVDDTQFLRFDSDAAIPRMEPREPWVEQEGPQYWEWTTGYAKA |
| | | NAQTDRVALRNLLRRYNQSEAGSHTLQGMNGCDMGPDGRLLRGYHQHAYDGKDYISLNEDLRSWTAAD |
| | | TVAQITQRFYEAEEYAEEFRTYLEGECLELLRRYLENGKETLGGSHHHHHHLE |
| 834 | S184 | MGSHLSLLYHITAVTDPHGGTPSFFVSGWLGPQQYLSYSNRRSEAEPYGAWIWESQVSWYWEKETVDLKN |
| | | KQQLFLEALEVLGEGSYTLQGLLGCELGPDNASVPTAKFALNGEDFMSFDPKQGSWAGEWPEALRISTRW |
| | | QQQAEAVSKERTFLLTSCPQRLLGHLERGRGNLEWKEPGGSHHHHHHLE |
| 835 | S185 | EPHSLRYNLTVLSWDGSVQSGFLTEVHLDGQPFLRCDRQKCRAKPQGQWAEDVLGNKTWDRETRDLTGN |
| | | GKDLRMTLAHIKDQKEGLHSLQEIRVCEIHEDNSTRSSQHFYYDGELFLSQNLETKEWTMPQSSRAQTLAM |
| | | NVRNFLKEDAMKTKTHYHAMHADCLQELRRYLKSGVVGGSHHHHHH |
| 836 | S186 | MGRSHSLHYLFMGASEQDLGLSLFEALGYVDDQLFVFYDHESRRVEPRTPWVSSRISSQMWLQLSQSLKG |
| | | WDHMFTVDFWTIMENHNHSKESHTLQVILGCEMQEDNSTEGYWKYGYDGQDHLEFCPDTLDWRAAEPR |
| | | AWPTKLEWERHKIRARQNRAYLERDCPAQLQQLLELGRGVGGSHHHHHH |
| 837 | S187 | MGRTHSLRYFRLGVSDPIHGVPEFISVGYVDSHPITTYDSVTRQKEPRAPWMAENLAPDHWERYTQLLRG |
| | | WQQMFKVELKRLQRHYNHSGSHTYQRMIGCELLEDGSTTGFLQYAYDGQDFLIFNKDTLSWLAVDNVAH |
| | | TIKQAWEANQHELLYQKNWLEEECIAWLKRFLEYGKGGSHHHHHH |
| 838 | S188 | MGSDGLQRLHMLQISYFRDPYHVWYQGNASLGGHLTHVLEGPDTNTTIIQLQPLQEPESWARTQSGLQSY |
| | | LLQFHGLVRLVHQERTLAFPLTIRCFLGCELPPEGSRAHVFFEVAVNGSSFVSFRPERALWQADTQVTSGVV |
| | | TFTLQQLNAYNRTRYELREFLEDTCVQYVQKHISAGGSHHHHHH |
| 839 | S189 | MGIKEEHVINQAEYCLNPDQSGEFKFDFDGDEIFHVDMAKKETVWRLEEFGRFASFEAQGALAHIAVDCA |
| | | NLEIMTKRSNYTPMGDTRPRFLWQLKYECHFFNGTERVRLNERCIYNQEESVRFDSDVGEYRAVTELGRPD |
| | | AEQWNSQKDHLEQRRAAVDTKCRHNYGVGESFTVQGGSHHHHHH |
| 840 | S190 | MGKEEHVIIQAEFCLNPDQSGEFMFDFDGDEIFHVDMAKKETVWRLEEFGRFASFEAQGALANIAVDCANL |
| | | EIMTKRSNYTPITNTRPRFLWQPKRECHFFNGTERVRFLDRYFYNQEESVRFDSDVGEFRAVTELGRPDAEY |
| | | WNSQKDILEQARAAVDTYCRHNYGVVESFTVQRRGGSHHHHHH |
| 841 | S191 | MGSKARMATGALAQAGGGGSGGGGSMIKEEHVINQAEYYLNPDQSGEFKFDFDGDEIFHVDMAKKETV |
| | | WRLEEFGRFASFEAQGALAHIAVDKANLEIMTKRSNYTPMGDTRPRFLWQLKYECHFFNGTERVRLNERCI |
| | | YNQEESVRFDSDVGEYRAVTELGRPDAEQWNSQKDHLEQRRAAVDTKCRHNYGVGESFTVQGGSHHHH |
| | | HH |
| 842 | S192 | MGKPVSKMRMATPLLMQALPMASGGGGSGGGGMIKEEHVINQAEYYLNPDQSGEFKFDFDGDEIFHVDM |
| | | AKKETVWRLEEFGRFASFEAQGALAHIAVDKANLEIMTKRSNYTPMGDTRPRFLWQLKYECHFFNGTERV |
| | | RLNERCIYNQEESVRFDSDVGEYRAVTELGRPDAEQWNSQKDHLEQRRAAVDTKCRHNYGVGESFTVQG |
| | | GSHHHHHH |
| 843 | S193 | MGSKARMATGALAQAGGSGGSMIKEEHVINQAEYYLNPDQSGEFKFDFDGDEIFHVDMAKKETVWRLEE |
| | | FGRFASFEAQGALAHIAVDKANLEIMTKRSNYTPMGDTRPRFLWQLKYECHFFNGTERVRLNERCIYNQEE |
| | | SVRFDSDVGEYRAVTELGRPDAEQWNSQKDHLEQRRAAVDTKCRHNYGVGESFTVQGGSHHHHHH |
| 844 | S194 | MGIKEEHVINQAEYYLNPDQSGEFKFDFDGDEIFHVDMAKKETVWRLEEFGRFASFEAQGALAHIAVDKA |
| | | NLEIMTKRSNYTPMGGGSKARMATGALAQAGGSGGSGDTRPRFLWQLKYECHFFNGTERVRLNERCIYN |
| | | QEESVRFDSDVGEYRAVTELGRPDAEQWNSQKDHLEQRRAAVDTKCRHNYGVGESFTVQGGSHHHHHH |
| 845 | S195 | MGKEEHVINQAEYYLNPDQSGEFKFDFDGDEIFHVDMAKKETVWRLEEFGRFASFEAQGALANIAVDKAN |
| | | LEIMTKRSNYTPITNTRPRFLWQPKRECHFFNGTERVRFNDRYFYNQEESVRFDSDVGEFRAVTESGRPDAE |
| | | YDNSQKDTLEVARAMVDTNCRHRYGHVESFTVQRRGGSHHHHHH |
| 846 | S196 | MGKEEHVINQAEYYLNPDQSGEFKFDFDGDEIFHVDMAKKETVWRLEEFGRFASFEAQGALANIAVDKAN |
| | | LEIMTKRSNYTPITNTRPRFLWQPKRECHFFNGTERVRFNDRYFYNQEESVRFDSDVGEFRAVTEPGRPEAE |
| | | YRNSQKDMLEAARAQVDTMCRHTYGQVESFTVQRRGGSHHHHHH |
| 847 | S197 | MAPTPMWPDDLQNHTFLHTVYCQDGSPSVGLSEAYDEDQLFFFDFSQNTRVPRLPEFADWAQEQGDAPAI |
| | | LFDKEFCEWMIQQIGPKLDGKIPGGFVAHVESTCLLDDAGTPKDFTYCISFNKDLLTCWDPEENKMAPCEF |
| | | GVLNSLANVLSQHLNQKDTLMQRLRNGLQNCATHTQPFWGSLTNRTRPGGSHHHHHH |
| 848 | S198 | MVKEEHVINQAEYYLNPDQSGEFKFDFDGDEIFHVDMAKKETVWRLEEFGRFASFEAQGALANIAVDKAN |
| | | LEIMTKRSNYTPMGDTRPRFLWQLKFECHFFNGTERVRLNERCIYNQEESVRFDSDVGEYRAVTELGRPDA |
| | | EYWNSQKDLLEQRRAAVDTYCRHNYGVGESFTVQGGSHHHHHHLE |
| 849 | S199 | MGIKEEHSIRQNEKYLNPDQSGNFEFHLDGQPIFRVDMQKKRTKWQLEEFGRFASFEAQGALANIAVDKA |
| | | NLEIMTKRSNQSEAGSHSWQLNFVCEFHEGTESVRLSERCYYNQENSVSFNSDVGEYRAVTELGRPDAEY |
| | | WNSQKDLLEQRRAAVDTYCRHNYGVGESFTVQRRGGSHHHHHHLE |
| 850 | S200 | MVKEEHVINQAEYYLNPDQSGEFKFDFDGDEIFHVDMAKKETVWRLEEFGRFASFEAQGALANIAVDKAN |
| | | LEIMTKRSNYTPMGDTRPRFLWQLKFECHFFNGTERVRLNERCIYNQEESVRFDSDVGEYRAVTELGRPDA |
| | | EYWNSQKDLLEQRRAAVDTYCRHNYGVGESFTVQGGSHHHHHHLE |
| 851 | S201 | MGIKEEHSIRQNEKYLNPDQSGNFEFHLDGQPIFRVDMQKKRTKWQLEEFGRFASFEAQGALANIAVDKA |
| | | NLEIMTKRSNQSEAGSHSWQLNFVCEFHEGTESVRLSERCYYNQENSVSFNSDVGEYRAVTELGRPDAEY |
| | | WNSQKDLLEQRRAAVDTYCRHNYGVGESFTVQRRGGSHHHHHHLE |
| 852 | S202 | MGSHLSLLYHITAVTDPHGGTPSFFVSGWLGPQQYLSYSNRRSEAEPYGAWIWESQVSWYWEKETVDLKN |
| | | KQQLFLEALEVLGEGSYTLQGLLGCELGPDNASVPTAKFALNGEDFMSFDPKQGSWAGEWPEALRISTRW |
| | | QQQAEAVSKERTFLLTSCPQRLLGHLERGRGNLEWKEPVSGWRLFKKISGKQKGSGSGEK |
| 853 | S203 | MGRSHSLHYLFMGASEQDLGLSLFEALGYVDDQLFVFYDHESRRVEPRTPWVSSRISSQMWLQLSQSLKG |
| | | WDHMFTVDFWTIMENHNHSKESHTLQVILGCEMQEDNSTEGYWKYGYDGQDHLEFCPDTLDWRAAEPR |
| | | AWPTKLEWERHKIRARQNRAYLERDCPAQLQQLLELGRGVVSGWRLFKKISGKQKGSGSGEK |
| 854 | S204 | IKEEHVIIQAEFYLNPDQSGEFQFDFDGDEIFHVDMAKKETVWRLEEFGRFASFEAQEALANIAVDKAKLEI |
| | | MTKRSNYTPMGDTRPRFLSQSKFECHFFNGTERVRLNERCIYNQEESVRFDSDVGEYRAVTELGRPDAEY |
| | | WNSQKDLLEQRRAAVDEYCRHNYGVGESFTVQRGGSHHHHHHLE |
| 855 | S205 | IKEEHVIIQAEFYLNPDQSGEFQGDFDGDEIFHVDMAKKETVWRLEEFGRQASFEAQGALANIAVDKANLEI |
| | | MTKRSNYTPMGDTRPRFLWQLKFECHFFNGTERVRLNERCIYNQEESVRFDSDVGEYRAVTELGRPDAEK |
| | | WNSQKDLLEQRRAAVDLHCRHNYGVGESFTVQRGGSHHHHHHLE |
| 856 | S206 | IKEEHVIIQAEFYLNPDQSGEFKFDFDGDEIFHVDMAKKETVWRLEEFGRFASFEAQRALANIAVDKANLEI |
| | | MTKRSNYTPMGDTRPRFLWQLKFECHFFNGTERVRLLERCIYNQEESVRFDSDVGEYRAVTELGRPDAEY |
| | | WNSQKDLLEQRRAAVDTQCRHNYGVGESFTVQRRVEGGSHHHHHHLE |
| 857 | S207 | IKEEHVIIQAEFYLNPDQSGEFKFDFDGDEIFHVDMAKKETVWRLEEFGRFASFEAQGALANIAVDKANLEK |
| | | MTKRSNYTPMGDTRPRFLWQLKFECHFFNGTERVRLLERCIYNQEESVRFDSDVGEYRAVTELGRPDAEYS |
| | | NSQKDLLEQRRAAVDTQCRHNYGVGESFTVQRRVEGGSHHHHHHLE |
| 858 | S208 | SHSMRYNYTSVSRPGRGEPRFNSEGYVDDTQFVRFDSDAASPREEPRAPWIEQEGPEDWDRNTQIYKAQA |
| | | QTDRESLRNLRGYYNQSEAGSHTLQSQYGCDVGPDGRLLRGHDQYYYDGKDYISLNEDLRSWTAADTAA |
| | | QITQRKAEAARIAEQRRAYLEGECVEWLRRYLENGKDKLERADGGSHHHHHHLE |
| 859 | S209 | SHSMRANYTSVSRPGRGEPRFNSEGYVDDTQFVRFDSDAASPREEPRAPWIEQEGPEYWDRNTQIYKAQA |
| | | QTDRESLRNLRGYYNQSEAGSHTLQSQVGCDVGPDGRLLRGHDQVYYDGKDYISLNEDLRSWTAADTAA |
| | | QITQRKNEAAREAEQRRAYLEGECVEWLRRYLENGKDKLERADGGSHHHHHHLE |
| 860 | S210 | DTRPRFLWQLKFECHFFNGTERVRLLERCIYNQEESVRFDSDVGEYRAVTELGRPDAEKWNSQKDLLEQR |
| | | RAAVDTYCRHNYGVGESFTVQRRSGGGIKEEHVIIQAEYYLNPDQSGEFQVDFDGDEIFHVDMAKKETVW |
| | | RLEEFGRFASFEAQLALANIAVDKANLEIMTKRSNYTPGGSHHHHHHLE |
| 861 | S211 | DTRPRFLWQLKFECHFFNGTERVRLLERCIYNQEESVRFDSDVGEYRAVTELGRPDAEYVNSQKDQLEQRR |
| | | AAVDTYCRHNYGVGESFTVQRRSGGGIKEEHVIIQAEYYLNPDQSGEFQFDFDGDEIFHVDMAKKETVWR |
| | | LEEFGRFASFEAQGALANIAQDKHNLEIMTKRSNYTPGGSHHHHHHLE |
| 862 | S212 | IKEEHSIRQNEQYLNPDQSGNFEFHLDGQPIFRVDMQKKRTKWQLEEFGRFASFEAQEALAGIAVDKANLEI |
| | | MTKRSNQSEAGSHSWQLNFVCEFHEGTESVRLSERCYYNQELSVSFNSDVGEYRAVTELGRPDAEYKNSQ |
| | | KDLLEQRRAAVDTYCRHNYGVGESFTVQRRGGSHHHHHHLE |
| 863 | S213 | IKEEHSIRQNEQYLNPDQSGNFEFHLDGQPIFRVDMQKKRTKWQLEEFGRFASEEAQGALANIAHDKANLE |
| | | IMTKRSNQSEAGSHSWQLNFVCEFHEGTESVRLSERCYYNQELSVSFNSDVGEYRAVTELGRPDAEKANSQ |
| | | KDNLEQRRAAVDTYCRHNYGVGESFTVQRRGGSHHHHHHLE |
| 864 | S214 | PENYLFQGRQECYAFNGTQRFLERANYNREEFVRFDSDVGEFRAVTELGRPDEEYWNSQKDILEEERAVPD |
| | | RACRHNYELGGPMTLQRRVQPRVNIKADHVSTYAAYVQTHRPTGEFKFEFDEDEMFYVDLDKKETVWHL |
| | | EEFGQAFSFEAQGGLANIAILNNNLNTLIQRSNHTQATNDPPHHHHHHLE |
| 865 | S215 | PENYLFQGRQECYAFNGTQRFLERYNYNREEIVRFDSDVGEFRAVTELGRPDEEYWNSQKDILEEERAVPD |
| | | RMCRHNYELGGPMTLQRRVQPRVNIKADHVSTYAAYVQTHRPTGEFKFEFDEDEMFYVDLDKKETVWHL |
| | | EEFGQAFSFEAQEGLALIAILNNNLNKLIQRSNHTQATNDPPHHHHHHLE |
| 866 | S216 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTERGRPTAEYY |
| | | NSQKDVLESARATVDTYCRHGYGRVESFTVQRRVQGGSHHHHHH |
| 867 | S217 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTEGGRPQAEY |
| | | QNSQKDVLEAARAGVDTTCRHRYGRVESFTVQRRVQ |
| 868 | S218 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTEKGRPQAEY |
| | | RNSQKDLLEHARADVDTPCRHRYGEVESFTVQRRVQ |
| 869 | S219 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTEGGRPMAEY |
| | | ANSQKDLLEHARAVVDTACRHSYGVVESFTVQRRVQ |
| 870 | S220 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTEGGRPPAEYR |
| | | NSQKDSLEKARASVDTLCRHPYGLVESFTVQRRVQ |
| 871 | S221 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTEFGRPLAEYS |
| | | NSQKDALERARAMVDTSCRHRYGPVESFTVQRRVQ |
| 872 | S222 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTEMGRPVAEY |
| | | ANSQKDFLESARAKVDTTCRHRYGRVESFTVQRRVQ |
| 873 | S223 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTEPGRPIAEYR |
| | | NSQKDSLELARARVDTSCRHRYGMVESFTVQRRVQ |
| 874 | S224 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTETGRPAAEYT |
| | | NSQKDLLESARAIVDTVCRHLYGLVESFTVQRRVQ |
| 875 | S225 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTEDGRPYAEY |
| | | HNSQKDYLEIARAYVDTMCRHFYGSVESFTVQRRVQ |
| 876 | S226 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTEKGRPAAEY |
| | | LNSQKDLLEWARAFVDTDCRHFYGSVESFTVQRRVQ |
| 877 | S227 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTEFGRPYAEYG |
| | | NSQKDLLEVARAFVDTVCRHQYGQVESFTVQRRVQGGSHHHHHH |
| 878 | S228 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTERGRPAAEY |
| | | RNSQKDKLEPARAVVDTMCRHPYGLVESFTVQRRVQ |
| 879 | S229 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTEPGRPMAEY |
| | | LNSQKDQLEVARAIVDTRCRHTYGTVESFTVQRRVQ |
| 880 | S230 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTEGGRPHAEY |
| | | VNSQKDELEWARAQVDTTCRHLYGMVESFTVQRRVQ |
| 881 | S231 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTEVGRPIAEYW |
| | | NSQKDTLELARAAVDTLCRHGYGLVESFTVQRRVQ |
| 882 | S232 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTEYGRPGAEY |
| | | GNSQKDKLEEARALVDTQCRHLYGVVESFTVQRRVQGGSHHHHHH |
| 883 | S233 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTERGRPDAEYL |
| | | NSQKDRLETARADVDTPCRHGYGSVESFTVQRRVQ |
| 884 | S234 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTETGRPDAEY |
| | | GNSQKDALEDARAMVDTLCRHSYGVVESFTVQRRVQ |
| 885 | S235 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTEVGRPDAEY |
| | | LNSQKDQLEAARAVVDTMCRHVYGWVESFTVQRRVQ |
| 886 | S236 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTEDGRPDAEY |
| | | ANSQKDALESARASVDTSCRHRYGYVESFTVQRRVQGGSHHHHHH |
| 887 | S237 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTEGGRPDAEY |
| | | VNSQKDRLEEARARVDTLCRHGYGRVESFTVQRRVQ |
| 888 | S238 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTEFGRPDAEYV |
| | | NSQKDFLEHARARVDTFCRHGYGHVESFTVQRRVQ |
| 889 | S239 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTEYGRPDAEY |
| | | LNSQKDRLELARAGVDTGCRHYYGLVESFTVQRRVQ |
| 890 | S240 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTEFGRPDAEYL |
| | | NSQKDWLEKARAFVDTACRHRYGAVESFTVQRRVQGGSHHHHHH |
| 891 | S241 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTEIGRPDAEYA |
| | | NSQKDILEAARALVDTGCRHGYGMVESFTVQRRVQ |
| 892 | S242 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTESGRPTAEYL |
| | | NSQKDILEQARAAVDTHCRHPYGAVESFTVQRRVQGGSHHHHHH |
| 893 | S243 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTELGRPLAEYV |
| | | NSQKDLLERARATVDTDCRHNYGVVESFTVQRRVQ |
| 894 | S244 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTEYGRPFAEY |
| | | WNSQKDVLEQARAAVDTLCRHVYGLVESFTVQRRVQ |
| 895 | S245 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTELGRPSAEYY |
| | | NSQKDPLEHARAAVDTICRHSYGVVESFTVQRRVQ |
| 896 | S246 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTELGRPFAEYK |
| | | NSQKDFLEQARAIVDTQCRHWYGVVESFTVQRRVQ |
| 897 | S247 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTELGRPDAEY |
| | | VNSQKDVLEWARAHVDTPCRHVYGVVESFTVQRRVQ |
| 898 | S248 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTELGRPDAEYR |
| | | NSQKDFLEEARAAVDTVCRHGYGGVESFTVQRRVQ |
| 899 | S249 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTERGRPSAEY |
| | | WNSQKDTLELARAAVDTGCRHMYGVVESFTVQRRVQGGSHHHHHH |
| 900 | S250 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTELGRPGAEYP |
| | | NSQKDYLEWARAAVDTTCRHVYGVVESFTVQRRVQ |
| 901 | S251 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTELGRPNAEY |
| | | WNSQKDHLEGARAAVDTRCRHQYGRVESFTVQRRVQ |
| 902 | S252 | KEEHVIIQAEFYLNPDQSGEFMFDFDGDEIFHVDMAKKETVWRLEEFGRFASFEAQGALANIAVDKANLEI |
| | | MTKRSNYTPITNTRPRFLWQPKRECHFFNGTERVRFLDRYFYNQEESVRFDSDVGEFRAVTELGRPDAEYW |
| | | NSQKDILEQARAAVDTYCRHNYGVVESFTVQRRVQ |
| 903 | S253 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTELGRPDAEY |
| | | WNSQKDILEQARAAVDTYCRHNYGVVESFTVQRRVQ |
| 904 | S254 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTEFGRPNAEY |
| | | MNSQKDLLEVARAMVDTSCRHWYGQVESFTVQRRVQ |
| 905 | S255 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTEAGRPVAEY |
| | | RNSQKDMLESARARVDTECRHYYGGVESFTVQRRVQ |
| 906 | S256 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTEYGRPAAEY |
| | | LNSQKDWLEMARATVDTVCRHQYGIVESFTVQRRVQ |
| 907 | S257 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTELGRPFAEYS |
| | | NSQKDALEPARAFVDTYCRHTYGRVESFTVQRRVQ |
| 908 | S258 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTEHGRPRAEYE |
| | | NSQKDTLEQARADVDTGCRHDYGPVESFTVQRRVQGGSHHHHHH |
| 909 | S259 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTERGRPSAEYF |
| | | NSQKDHLEGARAHVDTICRHGYGAVESFTVQRRVQ |
| 910 | S260 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTEVGRPGAEYP |
| | | NSQKDELELARAAVDTKCRHAYGLVESFTVQRRVQ |
| 911 | S261 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTESGRPEAEY |
| | | WNSQKDSLEKARASVDTRCRHLYGMVESFTVQRRVQ |
| 912 | S262 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTEGGRPQAEY |
| | | QNSQKDVLEAARAGVDTTCRHRYGHVESFTVQRRVQ |
| 913 | S263 | KEEHVIIQAEYYLNPDQSGEFKFDFDGDEIFHVDMAKKETVWRLEEFGRFASFEAQGALANIAVDKANLEI |
| | | MTKRSNYTPITNTRPRFLWQPKRECHFYNGTERVRFLDRYYYNQEESVRFDSDVGEFRAVTEKGRPWAEY |
| | | ANSQKDKLENARALVDTLCRHMYGNVESFTVQRRVQ |
| 914 | | IKRSKKNSLALSLTADQMVSALLDAEPPILYSEYDPTRPFSEASMMGLLTNLADRELVHMINWAKRVPGFV |
| | | DLTLHDQVHLLEXAWLEILMIGLVWRSMEHPGKLLFAPNLLLDRNQGKXVEGMVEIFDMLLATSSRFRM |
| | | MNLQGEEFVCLKSIILLNSGVYTFEKDHIHRVLDKITDTLIHLMAKAGLTLQQQHQRLAQLLLILSHIRHMS |
| | | NKGMEHLYSMKXKNVVPLSDLLLEMLDAHRLHAP |
| 915 | | PYASLTEIEHLVQSVCKSYRETCQLRLEDLLRQRSNIFSREEVTGYQRKSMWEMWERCAHHLTEAIQYVVE |
| | | FAKRLSGFMELCQNDQIVLLKAGAMEVVLVRMCRAYNADNRTVFFEGKYGGMELFRALGCSELISSIFDFS |
| | | HSLSALHFSEDEIALYTALVLINAHRPGLQEKRKVEQLQYNLELAFHHHLCKTHRQSILAKLPPKGKLRSLC |
| | | SQHVERLQIFQHLHPIVVQAAFPPLYKELFS |
| 916 | | SMPSQMEHAMETMMFTFHKFAGDKGYLTKEDLRVLMEKEFPGFLENQKDPLAVDKIMKDLDQCRDGKV |
| | | GFQSFFSLIAGLTIACNDYFVVHMKQENLYFQGDSTVHEILSKLSLE |
| 917 | | SPELGFSISGGVGGRGNPFRPDDDGIFVTRVQPEGPASKLLQPGDKIIQANGYSFINIEHGQAVSLLKTFQNT |
| | | VELIIVREVGNGAKQEIRVRVEKDSSVPTNLEVVAATPTSLLISWDASYYGVSYYRITYGETGGNSPVQEFT |
| | | VPYSSSTATISGLKPGVDYTITVYAYSDYYGSHHYSPISINYRT |
| 918 | | EATLGSGNLRQAVMLPEGEDLNEWIAVNTVDFFNQINMLYGTITEFCTEASCPVMSAGPRYEYHWADPIKC |
| | | SAPKYIDYLMTWVQDQLDDETLFPSKIGVPFPKNFMSVAKTILKRLFRVYAHIYHQHFDSVMQLQEEAHLN |
| | | TSFKHFIFFVQEFNLIDRRELAPLQELIEKLGSKDR |
| 919 | | ASITVPLESIKPSNILPVTVYDQHGFRILFHFARDPLPGRSDVLVVVVSMLSTAPQPIRNIVFQSAVPKVMKV |
| | | KLQPPSGTELPAFNPIVHPSAITQVLLLANPQKEKVRLRYKLTFTMGDQTYNEMGDVDQFPPPETWGSL |
| 920 | | EDLIDGIIFAANYLGSTQLLSDKTPSKNVRQAQEAVSRIKAQKLTEVDLFILTQRIKVLNADTQETDHPLRTIS |
| | | YIADIGNIVVLARRRYKICHVFESEDAQLIAQSIGQAFSVAYQEFLRANGINP |
| 921 | | EARLEEAVNRWVLKFYFHEALRAFRGSRYGDFRQIRDIMQALLVRPLGKEHTVSRLLRVMQCLSRIEEGEN |
| | | LDCSFDMEELTPLESAINVLEMIKTEFTLTEAVVESSRKLVKEAAVIICIKNKEFEKASKILKKHMPTTQKLR |
| | | NDLLNIIREKNLAHPVIQNFSYETFQQKMLRFLESHLDDAEPYLLTMAKKAL |

The following table provides exemplary sequences comprised in a disordered region as described herein.

TABLE 7
Disordered region amino acid sequences

| | | | SEQ |
|---|---|---|---|
| | | | ID |
| Source | Name | Exemplary Amino Acid Sequence | NO: |
| MTOR | mTOR | ITNATTAATTAATATTTASTEGSNSESEAESTENSPTP | 922 |
| MTOR | mTOR | HKPLRHPGMPKGLAHQLASPGLTTLPEASD | 923 |
| SDC-1 | Syndecan 1 | DMQPGHHETSTPAGPSQADLHTPHTEDGG | 924 |
| AAT | α1-antitrypsin | ALVNYIFFKGK | 925 |
| CYSLT2R | cysteinyl leukotriene | MERKFMSLQPSISVSEMEPNGTFSNNNSRNCTIENFKREFFP | 926 |
| | receptor 2 | | |
| CYSLT2R | cysteinyl leukotriene | MERKFMSLQPSISVSEMEPNG | 927 |
| | receptor 3 | | |
| CYSLT2R | cysteinyl leukotriene | SISVSEMEPNGTFSNNNSRNC | 928 |
| | receptor 4 | | |
| CYSLT2R | cysteinyl leukotriene | TFSNNNSRNCTIENFKREFFP | 929 |
| | receptor 5 | | |
| CYSLT2R | cysteinyl leukotriene | GMERKFMSLQPSISVSEMEPNGTFSNNNSRNCTIENFKREFFP | 930 |
| | receptor 6 | | |
| KCNA6 | Potassium voltage-gated | QQQEQQPASGGGGQNGQQAMS | 931 |
| | channel subfamily A | | |
| | member 6 | | |
| TNFRSF13B | TNF receptor superfamily | LPPELRRQRSGEVENNSDNSGRYQ | 932 |
| | member 13B | | |
| CD74 | CLIP | PPKPVSKMRMATPLLMQALPMGALP | 933 |
| KAAG1 | Kidney Associated DCDC2 | PGAAAAHLPRWPPPQLAASRREA | 934 |
| | Antisense RNA 1 | | |
| CD40LG | CD40 ligand | NKEETKKENSFEMQKGDQNPQIAAH | 935 |
| CHRM4 | Muscarinic acetylcholine | MANFTPVNGSSGNQSVRLVTSSSHNRYETVE | 936 |
| | receptor M4 | | |
| MRGX2 | Mas-related gene 2 | MDPTTPAWGTESTTVNGNDQALLLLCGKETLIP | 937 |
| MLNR | Motilin receptor | MGSPWNGSDGPEGAREPPWPALPPCDERRCSPFPL | 938 |
| C5AR1 | C5a anaphylatoxin | MDSFNYTTPDYGHYDDKDTLDLNTPVDKTSNTLRVPD | 939 |
| | chemotactic receptor | | |
| SSTR5 | Somatostatin receptor | MEPLFPASTPSWNASSPGAASGGGDNRTLVGPAPSAGA | 940 |
| | type 5 | | |
| H3R | Histamine H3 receptor | MERAPPDGPLNASGALAGEAAAAGGARGFSAAWTAVLAA | 941 |
| BDKRB1 | B1 bradykinin receptor | MASSWPPLELQSSNQSQLFPQNATACDNAPEAWDLLHRVL | 942 |
| BRS3 | Bombesin receptor subtype- | MAQRQPHSPNQTLISITNDTESSSSVVSNDNTNKGWSGDNS | 943 |
| | 3 | | |
| HCRTR1 | Orexin receptor type 1 | MEPSATPGAQMGVPPGSREPSPVPPDYEDEFLRYLWRDYLYPKQYE | 944 |
| ADRA2A | Adrenergic receptor alpha- | MFRQEQPLAEGSFAPMGSLQPDAGNASWNGTEAPGGGARATPYSLQV | 945 |
| | 2A | T | |
| HTR1B | 5-HT 1B receptor ( | MEEPGAQCAPPPPAGSETWVPQANLSSAPSQNCSAKDYIYQDSISLPW | 946 |
| | | K | |
| NPY2R | Neuropeptide Y receptor | MGPIGAEADENQTVEEMKVEQYGPQTTPRGELVPDPEPELIDSTKLIEV | 947 |
| | type 2 | QVW | |
| NPSR1B | Neuropeptide Y receptor | MPANFTEGSFDSSGTGQTLDSSPVACTETVTFTEVVEGKEWGSFYYSF | 948 |
| | type 2b | KTEQ | |
| AVPR1A | Vasopressin V1b receptor | MRLSAGPDAGPSGNSSPWWPLATGAGNTSREAEALGEGNGPPRDVRN | 949 |
| | | EELAK | |
| PTGER3 | Prostaglandin E2 receptor | MKETRGYGGDAPFCTRLNHSYTGMWAPERSAEARGNLTRPPGSGEDC | 950 |
| | EP3 | GSVSVA | |
| HCRTR2 | Orexin receptor type 2 | MSGTKLEDSPPCRNWSSASELNETQEPFLNPTDYDDEEFLRYLWREYL | 951 |
| | | HPKEYE | |
| CCKBR | Gastrin/cholecystokinin | MELLKLNRSVQGTGPGPGASLCRPGAPLLNSSSVGNLSCEPPRIRGAG | 952 |
| | type B receptor | TRELELAIRW | |
| NTSR1 | Neurotensin receptor | MRLNSSAPGTPGTPAADPFQRAQAGLEEALLAPGFGNASGNASERVL | 953 |
| | type 1 | AAPSSELDVNTDIYSKVLVTW | |
| LPAR1 | Lysophosphatidic Acid | MAAISTSIPVISQPQFTAMNEPQCFYNESIAFFYNRSGKHLATEWN | 954 |
| | Receptor 1 | | |
| AGTR1 | Angiotensin II receptor | MILNSSTEDGIKRIQDDCPKAGRHN | 955 |
| | type-2 | | |
| MRGPRX1 | Mas-related gene 1 | MDPTISTLDTELTPINGTEETLCYKQTLSLT | 956 |
| PTGER4 | Prostaglandin E2 Receptor | MSTPGVNSSASLSPDRLNS | 957 |
| | EP4 Subtype | | |
| HTR1A | 5-HT 1A receptor | MDVLSPGQGNNTTSPPAPFETGGNT | 958 |
| HTR2A | 5-Hydroxytryptamine | MDILCEENTSLSSTTNSLMQLNDDTRLYSNDFNSGEANTSDAFNWTVD | 959 |
| | Receptor 2A | SENRTNLSCEGCLSPS | |
| ADRA2C | Adrenoceptor Alpha 2C | MASPALAAALAVAAAAGPNASGAGERGSGGVANASGASWG | 960 |
| ADRB1 | Adrenoceptor Beta 1 | MGAGVLVLGASEPGNLSSAAPLPDGAATAARLLVPASPPASLLPPASE | 961 |
| | | SPEPL | |
| ADRB2 | Adrenoceptor Beta 2 | MGQPGNGSAFLLAPNGSHAPDHDVTQERDEVWVV | 962 |
| GPR55 | G Protein-Coupled Receptor | MSQQNTSGDCLFDGVNELMKT | 963 |
| | 55 | | |
| DRD3 | Dopamine Receptor D3 | MASLSQLSGHLNYTCGAENSTGASQARPHAYY | 964 |
| CNR1 | Cannabinoid Receptor 1 | MKSILDGLADTTFRTITTDLLYVGSNDIQYEDIKGDMASKLGYFPQKF | 965 |
| | | PLTSFRGSPFQEKMTAGDNPQLVPADQVNITEFYNKSLSSFKENEENI | |
| | | QCGENFMDIECFMVLNPSQQ | |
| MTNR1A | Melatonin Receptor 1A | MQGNGSALPNASQPVLRGDGARPSWLASA | 966 |
| PTGER2 | Prostaglandin E2 receptor | MGNASNDSQSEDCETRQWLPPGES | 967 |
| | EP2 subtype | | |
| P2RY12 | Purinergic Receptor P2Y12 | MQAVDNLTSAPGNTSLCTRDYKITQVLF | 968 |
| CCR1 | C-C Motif Chemokine | METPNTTEDYDTTTEFDYGDATPCQKVNERAFGAQ | 969 |
| | Receptor 1 | | |
| CYSLTR1 | Cysteinyl Leukotriene | MDETGNLTVSSATCHDTIDDFRNQVYST | 970 |
| | Receptor 1 | | |
| MRGPRX4 | MAS Related GPR Family | MDPTVPVFGTKLTPINGREETPCYNQTLSFTV | 971 |
| | Member X4 | | |
| UTS2R | Urotensin-2 receptor (UR- | MALTPESPSSFPGLAATGSSVPEPPGGPNATLNSSWASPTEPSSLEDL | 972 |
| | II-R) | VATGTI | |

The following examples are included for illustrative purposes only and are not intended to limit the scope of the invention.

### EXAMPLES

### Example 1. Detecting a Bioanalyte Using Engineered Scaffold Protein

An engineered scaffold protein generated from the scaffolds disclosed herein can target a specific protein that is present on only one type of cells' information about the type of cells, and quantitative measures of cell counts and relative ratio of different types of cells can be determined. For example, by identifying the population of T cells and their relative counts compared to B cells, an engineered scaffold is designed for CD28 (CTLA) present on T cells, but not on B Cells, and CD22, which is present on B cells and not T cells. Each engineered scaffold is labeled with a different fluorophore or a different peptide tag that binds to a fluorescent antibody. Labeled binders are incubated with a population of T cells and B cells and are subsequently washed to be rid of free binders. Flow cytometry is then used to sort and separate out the cells with two different colors. A quantitative assessment on the different types of cells may then follow.

Similar assays can also be set up to interrogate the presence of other bioanalytes using fluorescently labeled engineered scaffolds or engineered scaffolds with peptide tags that can bind to fluorescent antibodies. For example, an engineered scaffold for a particular target protein expressed by am mRNA, which may be a biomarker for a cell, can be designed, and the presence of particular cells can be determined in a population of cells. This can be made quantitative by combining the assay with single molecule microscopy.

### Example 2. Scaffold Engineering

The following non-limiting example demonstrates the development of an engineered scaffold that is designed to target a specific epitope.

C-myc is an oncogene that is a well validated cancer target, which is very difficult to drug because of its intrinsic disorder. To target this disordered region, the sequence EPLVLHE (SEQ ID NO: 996) that is present in C-myc in its disordered region is identified as the target epitope.

TEV protease that lacks its catalytic activity (C151A) is chosen as the scaffold for the binder development. Library of variants (up to a theoretical maximum of 108) is generated using Twist Biosciences' SOLD approach of spreading the variants across the full length of the enzyme sequence, maintaining the C151A mutation.

The library of variants are cloned into a yeast vector and fused to Aga1 protein at either the N-terminus or at the C-terminus along with a 6×Histidine tag (SEQ ID NO: 979) at the C-terminal end of the Aga1-TEV variant fusion, and transfected in yeast cells.

Functionally expressed and displayed variants are incubated with magnetic beads carrying the recognition peptide EPLVLHE (SEQ ID NO: 996) and selected for binding. Binders from this selection were washed out and labeled with the tag containing protein and subjected to FACS sorting to separate binders from non-binders.

From the first round of selection, positive binders and negative binders are separated out and yeast cells are subjected to NGS sequencing to identify the binders and non-binders. The binders from the first round of selection are grown up and further subjected to successive rounds of screening with more stringent binding conditions to arrive at a potent binder for the target sequence.

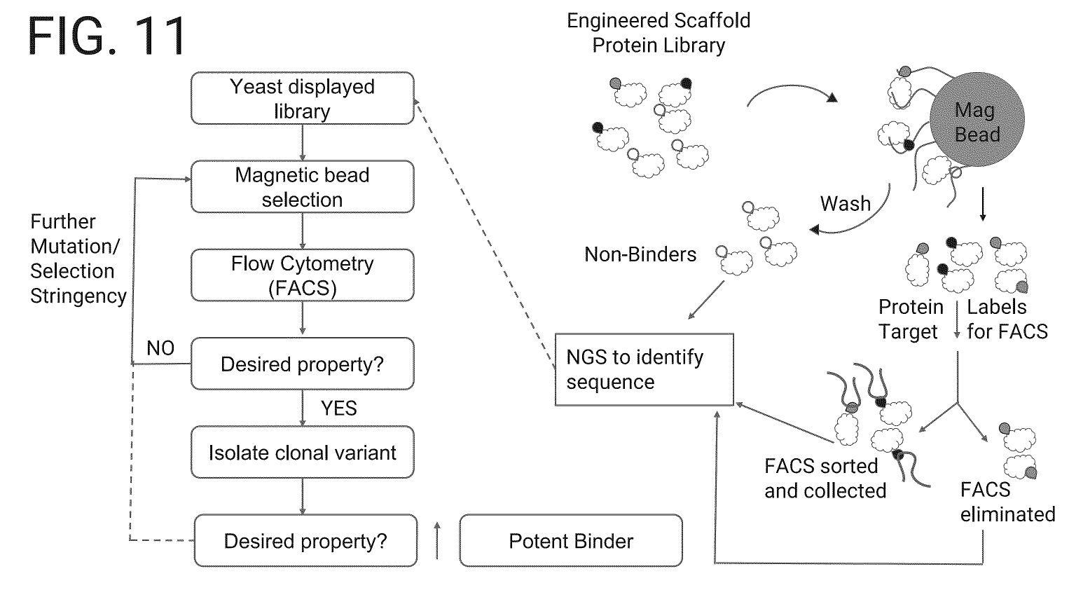

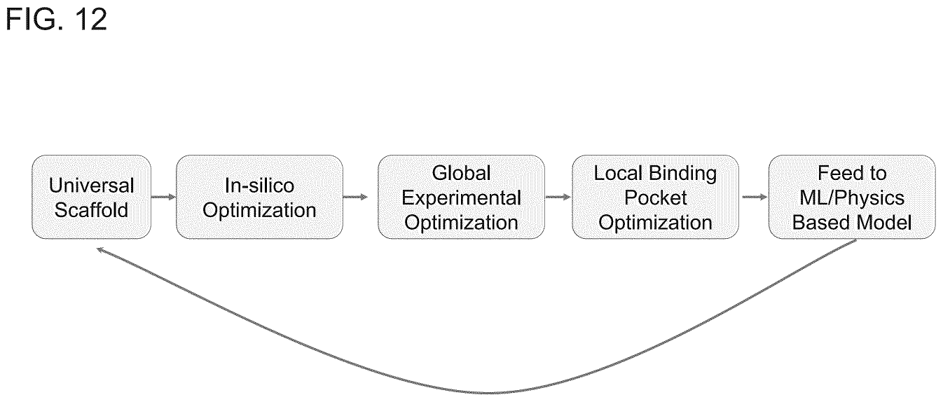

Data generated from each round of selection are used to generate a database of engineered scaffold proteins and their relative strengths for the target. A flow-chart demonstrating the scaffold engineering process can be seen in FIG. 11.

### Example 3. Binder Development Pipeline

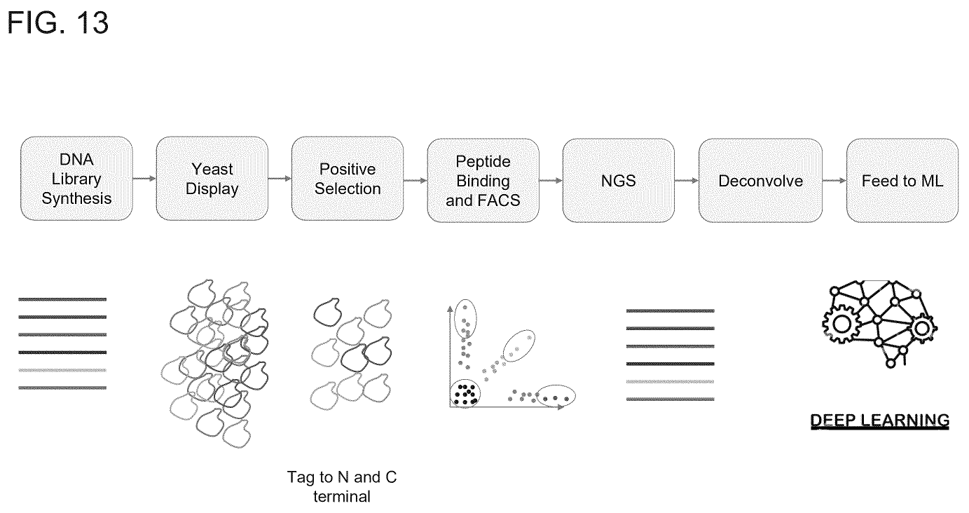

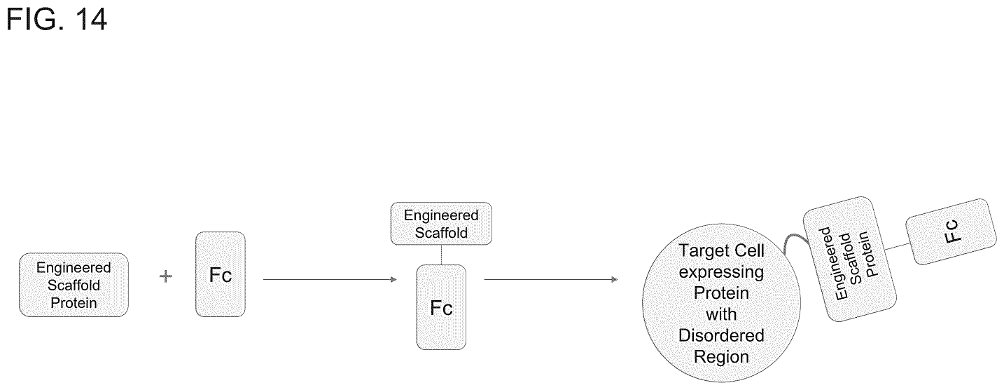

A non-limiting example of the binder development pipeline is displayed in FIG. 13. Yeast display of cell surface protein scaffolds is performed using well known methods such as those described in Angelini et al 2015, Methods in molecular Biology, ‘Protein Engineering and Selection Using Yeast Surface Display’. This procedure involves starting with the design of engineered scaffold libraries for each scaffold protein. The yeast strain with Aga1 gene stably integrated into the chromosome is chosen. The nucleic acids encoding engineered scaffold proteins, fused with the Aga2 gene, is cloned into a circular yeast display vector using Gibson assembly and further established protocols. The yeast library is transformed into competent cells and selected using a URA3 complementation method. The yeast library cells are initially grown in YPD medium, which are subsequently used to inoculate SD-CAA medium. Cells are passaged once and induced, where the induced cells are then used to screen for binders. Screening is done in two steps using a magnetic bead-based screening followed by flow cytometry.

In the first step, a negative selection for magnetic bead binders is performed to deplete the library of streptavidin coated magnetic bead binders by incubating the library with magnetic beads that do not have the immobilized target. The flow through from the negative selection is then incubated with magnetic beads that have the target immobilized via biotin streptavidin linkage. After stringent washing to remove non-binders, the beads were collected and the binder library was isolated.

Selected yeast cells from the magnetic beads screening step are further subjected to fluorescence-activated cell sorting (FACS) to enrich for higher affinity binders to target protein. Yeast display vectors are designed to have a tag epitope (His or FLAG) at the C terminus end of the engineered scaffold protein to be able to be labeled with an antibody. The target protein will be labeled with a different tag (His, FLAG, myc). Using a two color FACS sorting, only yeast cells showing engineered scaffold proteins binding to target proteins exhibits dual color and are collected. Repeated cycles of selection and screening yields potent binders to the target protein.

### Example 4. Monomeric MHC Designs

The following non-limiting example displays various engineered monomeric MHC constructs and designs for an engineered scaffold protein derived from MHC proteins. The engineered scaffold proteins may be generated in accordance with any of the methods and examples disclosed herein.

TABLE 8
MHC Specific Sequences for Chimera 1-4

| Molecule | | Sequence |
|---|---|---|
| Type | Molecule Sequence | ID NO: |
| Chimera 1 | MIKEEHVIIQAEFYLNPDQSGEFMFDFDGDEIFHVDMAKKET | 636 |
| | VWRLEEFGRFASFEAQGALANIAVDKANLEIMTKRSNYTPIT | |
| | NVQSEAGSHTVQRMYGCDVGSDWRFLRGYHQYAYSDVGEYR | |
| | AVTELGRPDAEYWNSQKDLLEQRRAAVDTYCRHNYGVGESFTVQRR | |
| | VE | |
| Chimera 2-1 | MIKEEHVIIQAEFYLNPDQSGEFMFDFDGDEIFHVDMAKKET | 637 |
| | VWRLEEFGRFASFEAQGALANIAVDKANLEIMTKRSNYTPIT | |
| | NVGGGGSGGGGSGGGGSGDTRPRFLWQLKFECHFFNGTERVRLL | |
| | ERCIYNQEESVRFDSDVGEYRAVTELGRPDAEYWNSQKDLLEQRRAA | |
| | VDTYCRHNYGVGESFTVQRRVE | |
| Chimera 2-2 | MIKEEHVIIQAEFYLNPDQSGEFMFDFDGDEIFHVDMAKKET | 638 |
| | VWRLEEFGRFASFEAQGALANIAVDKANLEIMTKRSNYTPIT | |
| | NVGGGGGGGGGDTRPRFLWQLKFECHFENGTERVRLLERCIYNQE | |
| | ESVRFDSDVGEYRAVTELGRPDAEYWNSQKDLLEQRRAAVDTYCRH | |
| | NYGVGESFTVQRRVE | |
| Chimera 2-3 | MIKEEHVIIQAEFYLNPDQSGEFMFDFDGDEIFHVDMAKKET | 639 |
| | VWRLEEFGRFASFEAQGALANIAVDKANLEIMTKRSNYTPIT | |
| | NVGGGGGGGDTRPRFLWQLKFECHFFNGTERVRLLERCIYNQEES | |
| | VRFDSDVGEYRAVTELGRPDAEYWNSQKDLLEQRRAAVDTYCRHNY | |
| | GVGESFTVQRRVE | |
| Chimera 2-4 | MIKEEHVIIQAEFYLNPDQSGEFMFDFDGDEIFHVDMAKKET | 640 |
| | VWRLEEFGRFASFEAQGALANIAVDKANLEIMTKRSNYTPIT | |
| | NVGGGGSGDTRPRFLWQLKFECHFFNGTERVRLLERCIYNQEESVR | |
| | FDSDVGEYRAVTELGRPDAEYWNSQKDLLEQRRAAVDTYCRHNYGV | |
| | GESFTVQRRVE | |
| Chimera 2-5 | MIKEEHVIIQAEFYLNPDQSGEFMFDFDGDEIFHVDMAKKET | 641 |
| | VWRLEEFGRFASFEAQGALANIAVDKANLEIMTKRSNYTPIT | |
| | NVLEGDTRPRFLWQLKFECHFFNGTERVRLLERCIYNQEESVREDS | |
| | DVGEYRAVTELGRPDAEYWNSQKDLLEQRRAAVDTYCRHNYGVGES | |
| | FTVQRRVE | |
| Chimera 2-6 | MIKEEHVIIQAEFYLNPDQSGEFMFDFDGDEIFHVDMAKKET | 642 |
| | VWRLEEFGRFASFEAQGALANIAVDKANLEIMTKRSNYTPIT | |
| | NVGGGGSGGGGSGDTRPRFLWQLKFECHFFNGTERVRLLERCIYN | |
| | QEESVRFDSDVGEYRAVTELGRPDAEYWNSQKDLLEQRRAAVDTYC | |
| | RHNYGVGESFTVQRRVE | |
| Chimera 2-7 | MGDTRPRFLWQLKFECHFFNGTERVRLLERCIYNQEESVRFDSDVG | 643 |
| | EYRAVTELGRPDAEYWNSQKDLLEQRRAAVDTYCRHNYGVGESFTV | |
| | QRRVELEMIKEEHVIIQAEFYLNPDQSGEFMFDFDGDEIFHVD | |
| | MAKKETVWRLEEFGRFASFEAQGALANIAVDKANLEIMTKR | |
| | SNYTPITNV | |
| Chimera 2-8 | MGDTRPRFLWQLKFECHFFNGTERVRLLERCIYNQEESVRFDSDVG | 644 |
| | EYRAVTELGRPDAEYWNSQKDLLEQRRAAVDTYCRHNYGVGESFTV | |
| | QRRVEQSEAGSHMIKEEHVIIQAEFYLNPDQSGEFMFDFDGDEI | |
| | FHVDMAKKETVWRLEEFGRFASFEAQGALANIAVDKANLEI | |
| | MTKRSNYTPITNV | |
| Chimera 2-9 | MGDTRPRFLWQLKFECHFFNGTERVRLLERCIYNQEESVRFDSDVG | 645 |
| | EYRAVTELGRPDAEYWNSQKDLLEQRRAAVDTYCRHNYGVGESFTV | |
| | QRRVEQSEAGSHMIKEEHVIIQAEFYLNPDQSGEFMFDFDGDEI | |
| | FHVDMAKKETVWRLEEFGRFASFEAQGALANIAVDKANLEI | |
| | MTKRSNYTPITNVPPEVTVLTNSPVELREPNVLICFIDKFTPPV | |
| | VNVTWLRNGKPVTTGVSETVFLPREDHLFRKFHYLPFLPSTE | |
| | DVYDCRVEHWGLDEPLLKHWEFDA | |
| Chimera 2- | MIKEEHVIIQAEFYLNPDQSGEFMFDFDGDEIFHVDMAKKET | 646 |
| 10 | VWRLEEFGRFASFEAQGALANIAVDKANLEIMTKRSNYTPIT | |
| | NVLEGDTRPRFLWQLKFECHFFNGTERVRLLERCIYNQEESVREDS | |
| | DVGEYRAVTELGRPDAEYWNSQKDLLEQRRAAVDTYCRHNYGVGES | |
| | FTVQRRVEPKVTVYPSKTQPLQHHNLLVCSVSGFYPGSIEVRWFRNG | |
| | QEEKAGVVSTGLIQNGDWTFQTLVMLETVPRSGEVYTCQVEHPSVTS | |
| | PLTVEWRA | |
| Chimera 2- | MIKEEHVIIQAEFYLNPDQSGEFMFDFDGDEIFHVDMAKKET | 647 |
| 5-Fusion | VWRLEEFGRFASFEAQGALANIAVDKANLEIMTKRSNYTPIT | |
| | NVLEGDTRPRFLWQLKFECHFENGTERVRLLERCIYNQEESVREDS | |
| | DVGEYRAVTELGRPDAEYWNSQKDLLEQRRAAVDTYCRHNYGVGES | |
| | FTVQRRVESSGMKAIFVLNAQHDEAVDANSLAEAKVLANRELDKYGV | |
| | SDYYKNLINNAKTVEGVKALIDEILAALP | |
| Chimera 3 | GSHSMRYFFTSVSRPGRGEPRFIAVGYVDDTQFVREDSDAAS | 648 |
| | QRMEPRLEEFGRFASFEAQGALANIAVDKANLEIMTKRSNYT | |
| | PITNVLEGDTRPRFLWQLKFECHFFNGTERVRLLERCIYNQEESVRF | |
| | DSDVGEYRAADMAAQTTKHKWEAAHVAEQLRAYLEGTCVE | |
| | WLRRYLENGKETLQRTDAPKTHMTHHAVSDHEATLRCWAL | |
| | SFYPAEITLTWQRDGEDQTQDTELVETRPAGDGTFQKWAAV | |
| | VVPSGQEQRYTCHVQHEGLPKPLTLRWE | |
| Chimera 4-1 | MGDTRPRFLWQLKFECHFFNGTERVRLLERCIYNQEESVRFDSD | 649 |
| | VGEYRAVTELGRPDAEYWNSQKDLLEQRRAAVDTYCRHNYGV | |
| | GESFTVQRRVEQSEAGSHTVQRMYGCDVGSDWRFLRGYHQY | |
| | AYDGKDYIALKEDLRSWTAADMAAQTTKHKWEAAHVAEQL | |
| | RAYLEGTCVEWLRRYLENGKETLQRTDAPKTHMTHHAVSD | |
| | HEATLRCWALSFYPAEITLTWQRDGEDQTQDTELVETRPAG | |
| | DGTFQKWAAVVVPSGQEQRYTCHVQHEGLPKPLTLRWE | |
| Chimera 4-2 | MGSHSMRYFFTSVSRPGRGEPRFIAVGYVDDTQFVRFDSDAA | 650 |
| | SQRMEPRAPWIEQEGPEYWDGETRKVKAHSQTHRVDLGTL | |
| | RGYYNQSEAGSHGDTRPRFLWQLKFECHFFNGTERVRLLERCIYN | |
| | QEESVRFDSDVGEYRAVTELGRPDAEYWNSQKDLLEQRRAAVDTYC | |
| | RHNYGVGESFTVQRRVEPKVTVYPSKTQPLQHHNLLVCSVSGFYPGS | |
| | IEVRWFRNGQEEKAGVVSTGLIQNGDWTFQTLVMLETVPRSGEVYTC | |
| | QVEHPSVTSPLTVEWRA | |

Bold text indicates an MHC II alpha (a) chain sequence. Underlined text indicates a linker. Italicized text indicates an MHC II beta (B) sheet sequence. Bold and underlined text indicates an MHC I alpha chain sequence. Underlined and italicized text indicates a sequence for other protein fusions.

The specific linker sequences used in the MHC constructs are also listed below.

TABLE 9
Specific Linker Sequences

| | Linker Sequence | SEQ ID NO: |
|---|---|---|
| 1. | (GGGGS)n; where n = 1-5 | 973 |
| 2. | GGGGGG | 974 |
| 3. | GGGGGGGG | 975 |
| 4. | QSEAGSH | 976 |
| 5. | LE | 977 |
| 6. | SSG | 978 |

The (GGGGS)n (SEQ ID NO: 1008) linkers are flexible linkers and are believed to increase solubility and stability. The LE linker is believed to increase PK in an antibody (e.g., acts as a dipeptide linker). QSEAGSH (SEQ ID NO: 976) is an MHC I linker from the crystal structure of 2XPG. This is the linker between the alpha helix and the second part of the beta sheet.

The specific fusion partner sequences used in the MHC constructs disclosed herein are also listed below.

TABLE 10
Fusion Sequences

| Classification | Fusion Sequence | SEQ ID NO: |
|---|---|---|
| 6x His Tag | HHHHHH | 979 |
| FLAG Tag | (DYKDDDDK)n; where n = 1-3 | 980 |
| Strep Tag II | WSHPQFEK | 981 |
| TEV protease | ENLYFQS | 982 |
| cleavage site | | |
| Albumin Binding | MKAIFVLNAQHDEAVDANSLAEAKVLANRELD | 983 |
| Domain | KYGVSDYYKNLINNAKTVEGVKALIDEILAALP | |
| Albumin Binding | KTKNVVCNEELTELAEAKVLANRELDKYGVSDY | 984 |
| Domain | HKNLINNAKTVEGVKDLQAQVVESA | |
| Albumin Binding | MKAIFVLNAQHDEAVDANSALAKAKVLANREL | 985 |
| Domain | DKYGVSDYYKKLVNNAKTVEGVKDLQAQVVES | |
| | AKKAR | |
| Albumin Binding | KTKNVVCNEELTALAKAKVLANRELDKYGVSD | 986 |
| Domain | YYKKLVNKAKAAETVATLQAQVVESAKKAR | |

### Constructs for Chimera 1-4

The constructs for each of chimera 1-4 listed in TABLE 8 are described as follows.

Chimera 1. The construct for chimera 1 is shown below, where domain 1 and domain 3 can be a part of MHC II α chain or MHC II β chain, and domain 2 can be a part of an MHC I α chain.

Chimera 2. The construct for chimera 2 is shown below, where domain 1 can be a part of an MHC II α chain or MHC II β chain, and domain 2 can likewise be a part of a MHC II α chain or a MHC II β chain. The two domains are connected by a short peptide linker.

Chimera 3. The construct for chimera 3 is shown below, where domain 1 or domain 3 can be part of an MHC I α chain, while domains 2 and 4 can be part of an MHC II α/β chain and domains 2 and 3 are connected by a short peptide linker

Chimera 4. The construct for chimera 4 is shown below, where domain 1 can be part of an MHC I α chain or an MHC II α/β chain and similarly for domain 2.

All of the above constructs can have C or N terminal fusions that can include purification tags such as a His tag (HHHHHH (SEQ ID NO: 979)), FLAG tag, Myc Tag, GST tag, a solubility tag like SUMO, a protease cleavage tag such as TEV protease cleavage tag (ENLYFS (SEQ ID NO: 1009)) or Thrombin cleavage tag, a serum albumin binding domain or transferrin domain or transferrin binding domain.

### Example 5. Engineered Scaffold Protein Production from Inclusion Bodies

The present example describes methods for generating engineered scaffold proteins as described herein. E. coli pET28 vectors or pD451 vectors encoding engineered scaffold proteins of interest were transformed into BL21 DE3 cells. Overnight starter cultures were grown from single colonies, which were then used to seed 1 L cultures with appropriate antibiotics and incubated at 37 C. When the optical density reached 0.6-0.8, protein production was induced by addition of 1 mM IPTG and the flasks were maintained at 37 C with shaking for 16 hours. Subsequently the cells were harvested by centrifugation at 8000×g for 10 minutes, resuspended in Lysis buffer (50 mM Tris pH 8, 500 mM NaCl, 10 mM Imidazole) and lysed using a sonicator. The sonicator was set to pulse mode with 15 s on time and 45 s off time for a total of 10 cycles, while the cells were maintained in an ice bucket.

Cell lysate was subjected to centrifugation at 18000×g for 30 minutes to clarify the solution and the cell pellet containing the protein of interest in inclusion bodies was preserved while the supernatant was discarded. Pellets were resuspended in 10 mL of Urea Buffer (50 mM Tris pH 8, 500 mM NaCl, 10 mM Imidazole, 6M Urea) and allowed to incubate on a rotator at room temperature overnight to re-solubilize the protein of interest from inclusion bodies. The resolubilized solution was clarified once again to remove aggregates by centrifuging at 18000×g for 30 minutes and supernatant was saved. The re-solubilized protein was applied to a Nickel column that had previously been equilibrated with Lysis Buffer and allowed to bind under gravity flow. The column was then washed extensively with a wash buffer (50 mM Tris pH 8, 500 mM NaCl, 25 mM Imidazole) to remove Urea and refold the protein on the column. Protein of interest was eluted with an elution buffer (50 mM Tris pH 8, 500 mM NaCl, 250 mM Imidazole). Eluted protein was buffer exchanged into PBS, flash frozen and stored.

Engineered scaffold proteins generated by the above method include S001, S005-S007, S031, S043-S044, S049, S055-S060, S065-S069, S077-S078, S082, S090, S095-S096, S098-S102, S107, S117-S118, S132-S134, S139-S140, S143-S145, S161-S167, S169-S180, S183-S215, S227, S232, S236, S240, S242, S249, S258, and SEQ ID NO: 914.

### Example 6: Characterizing Engineered Scaffold Proteins

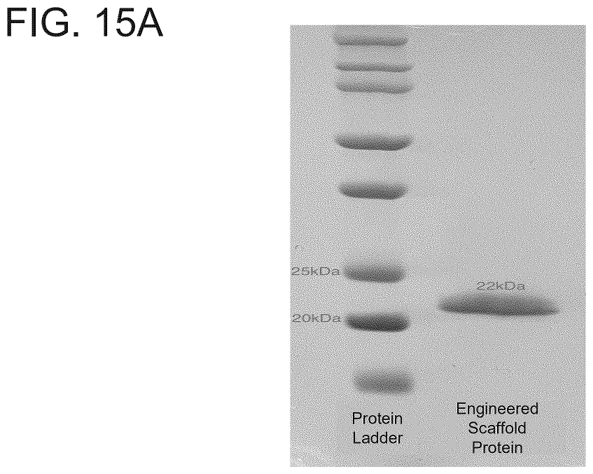

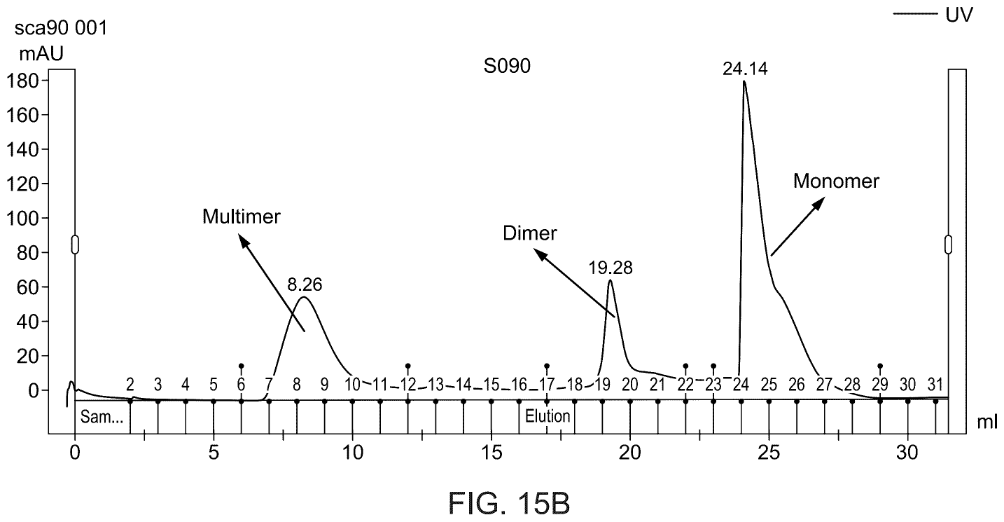

The present example characterizes generated engineered scaffold proteins as described herein. The molecular weight of engineered scaffold proteins generated from the method of Example 5 range from about 20 kDa to about 50 kDa. SDS PAGE was run on an engineered scaffold protein (S177 (SEQ ID NO: 827) which confirmed the expected size (21.92 kDa) on the gel and can be produced in soluable form as demonstrated by FIG. 15A.

To analyze the soluble proteins produced, an engineered scaffold protein (S090 (SEQ ID NO: 740)) were run on a Superdex 200 Increase 10/300 column by Cytiva, using an AKTA FPLC. The UV absorbance at 280 nM was recorded as a function of elution volume. Three peaks at different elution volumes were recorded for S090 and as seen in FIG. 15B. The results in FIG. 15B indicates that the engineered scaffold proteins are generated as a mixture of monomers, dimers and multimers with at least 50% of the protein existing as monomers.

### Example 7. Modified Sandwich ELISA Assay for Protein-Peptide Interactions

Disordered epitopes derived from target proteins with a biotin were synthesized either chemically or expressed in E. coli and subsequently labeled with a biotin using a sortase mediated ligation of a short biotinylated peptide.

Binder proteins were coated on the surface of a high binding capacity 96 well plate by incubating 100 μL of protein at 5 μg/mL in a coating solution (10 mM Tris pH 8.8, 50 mM NaCl), overnight at 4 C. The next morning, the coating solution was aspirated out and wells were washed once with 300 μL ELISA wash buffer (1×PBS, 0.05% Tween-20) and solution was aspirated out. The wells were blocked with 200 μL of a blocking buffer (1×PBS, 0.05% Tween-20, 3% BSA) at room temperature for 1 hour. After one hour, the blocking solution was aspirated out and 100 μL of biotinylated polypeptide in a binding buffer (1×PBS, 0.05% Tween-20, 0.5% BSA) at various concentrations was added to the appropriate well and incubated for 20 minutes. Peptide solution was aspirated out after 20 minutes and wells were washed 5 times with 300 μL of ELISA wash buffer. To detect the biotinylated peptide, 100 μL of streptavidin-HRP (50 ng/ml) was added to each well and incubated for 1 hour. The wells were subsequently washed 5 times with 300 μL of ELISA wash buffer. 100 μL of QuantaBlu Fluorogenic Peroxidase substrate solution was added to each well, incubated for 30 minutes and reaction stopped by adding 100 μL of stop solution. Fluorescent signal from each well was measured using a Biotek Cytation5 plate reader by exciting the fluorophore at 325 nM and measuring emission at 420 nM. Fluorescent signals were plotted as a function of polypeptide concentration.

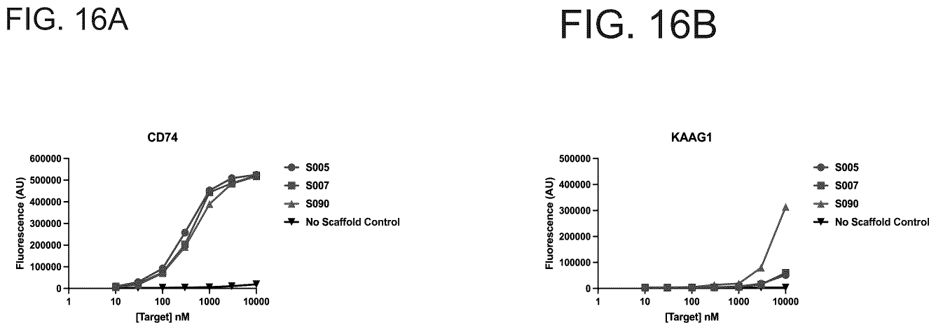

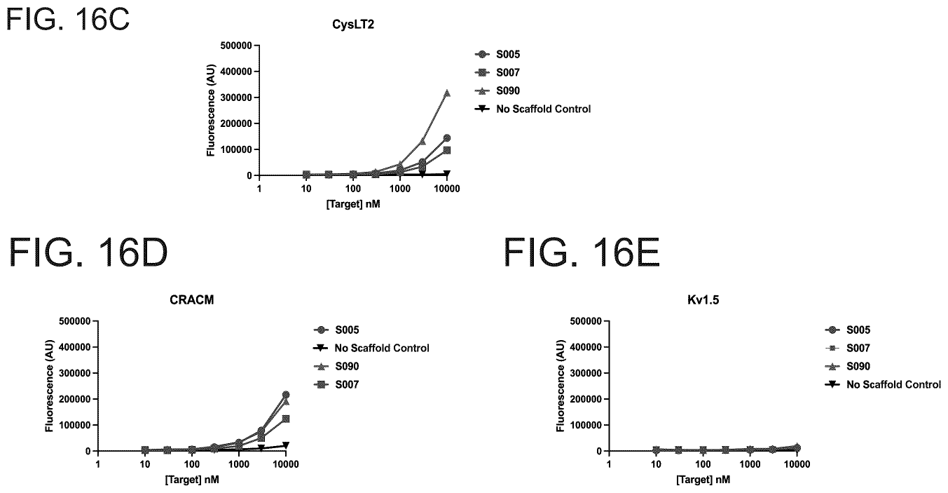

Engineered protein scaffolds S007, S005 and S090 were derived from MHC II molecules and the binding activities of these engineered scaffolds was measured in an ELISA assay as described herein. Results for engineered protein scaffolds S007, S005 and S090 binding increasing amounts of disordered regions of CD74, KAAG1, CysLT2, CRACM, and Kv1.5 relative to a negative control are shown in FIGS. 16A-E. In human cells, every MHC II based molecule is associated with a CLIP peptide derived from CD74 and accordingly, CD74 binding is used as a positive control. The data indicates that the engineered scaffold proteins can bind to long disordered polypeptides derived from extracellular disease target proteins covering GPCRs (e.g., CysLT2), ion channels (e.g., CRACM, Kv1.5) and membrane proteins (e.g., CD74, KAAG1).

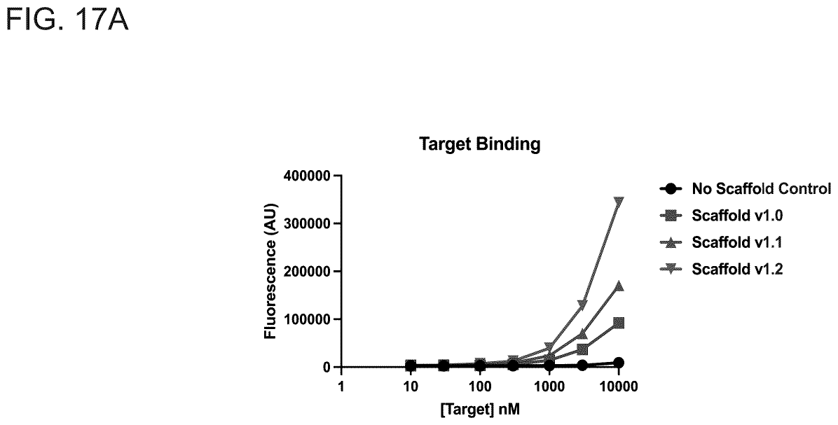

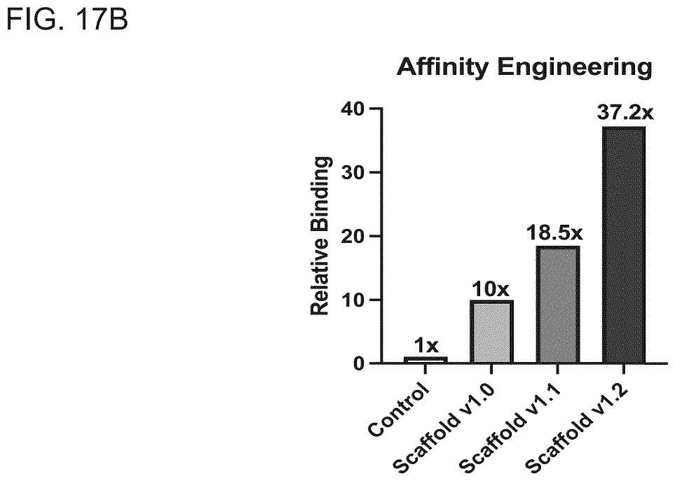

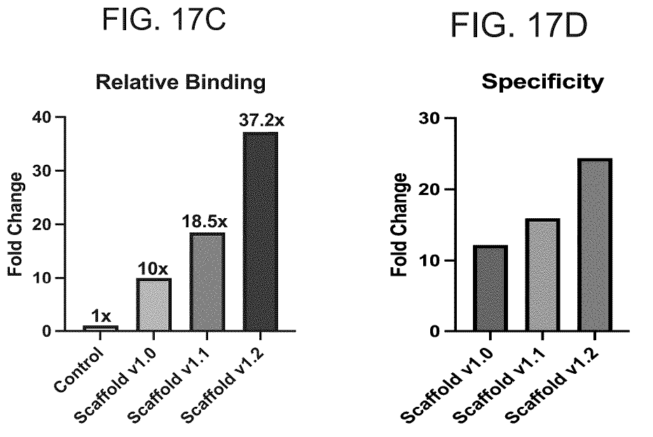

The results in FIG. 17A demonstrate that scaffold optimization and rational design increases binding of a target peptide, which is CysLT2 in this assay. In FIG. 17A, scaffold v1.0 refers to S007 which was derived from MHC II and comprises a binding unit (comprising an N-terminal alpha chain of 82 amino acids), a hinge unit (comprising an N-terminal beta chain of 98 amino acids) and an immunoglobulin unit (i.e., a beta-globulin domain), and includes a binding groove architecture as described herein. The binding unit is fused to the N terminal of the hinge unit, which is fused at its C terminal to the immunoglobulin unit. Scaffold v1.1 refers to S005, which is derived from S007 by removing the immunoglobulin unit. Scaffold v1.2 refers to S090, which was rationally designed based on v1.1 (S005), and includes amino acid alterations to increase binding efficiency of the target peptide without compromising the binding groove architecture.

Binding affinity, relative binding and binding specificity relative to the negative control was measured at 10,000 nM of the target peptide, results of which can be seen in FIG. 17B-D. The fluorescent binding signals were normalized to controls, which consisted of binding reactions without any scaffold. As such, data was derived from the binding isotherms by dividing the fluorescent signal at that particular target concentration for a particular engineered scaffold protein by the fluorescent signal for a control reaction which does not contain any scaffold protein. FIG. 17B indicates that scaffold optimization and rational design increases binding affinity of an engineered scaffold protein to a target peptide (CYSLTR2). Likewise, FIGS. 17C and 17D indicate that scaffold optimization and rational design increases relative binding and binding specificity of an engineered scaffold protein to a target peptide (CYSLTR2), respectively.

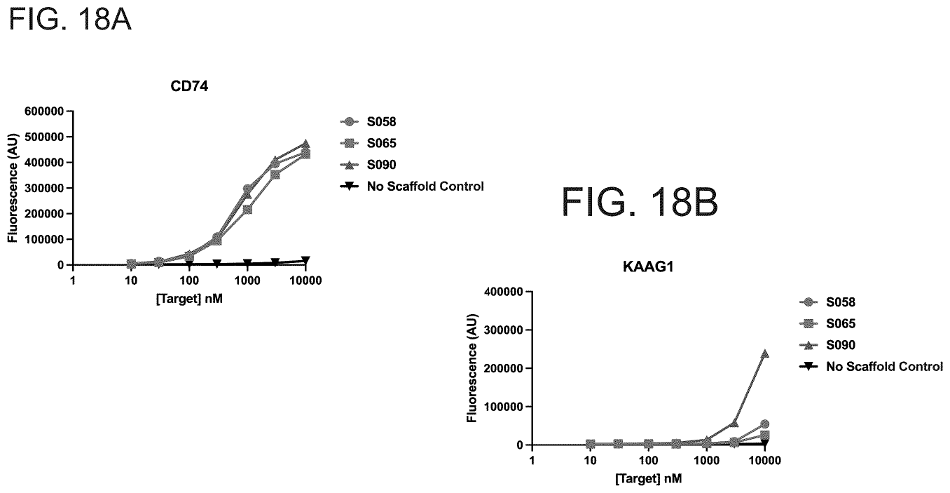

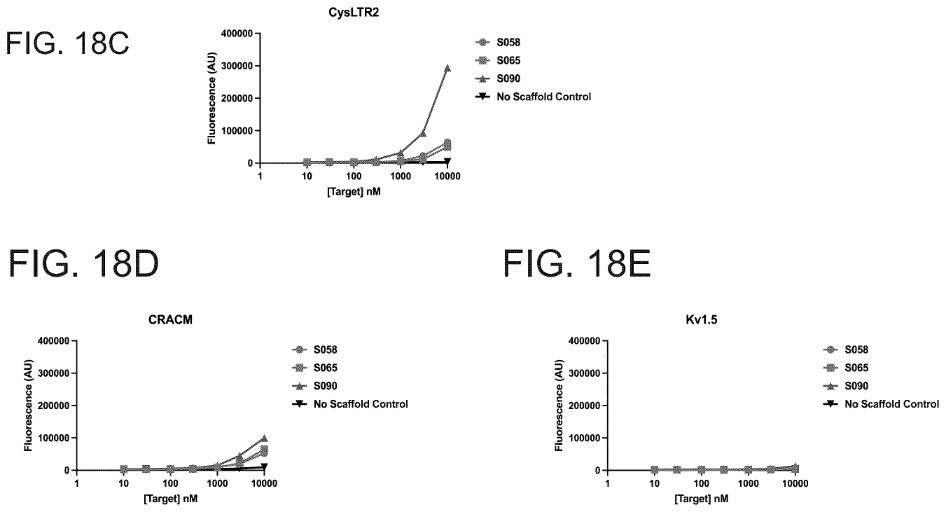

Engineered scaffolds S058 and S065 were derived from MHC I proteins. The binding activities of these engineered scaffolds were measured in an ELISA assay as described herein, relative to S090, which was derived from MHC II proteins, and relative to a negative control. Results for binding of CD74 (positive control), KAAG1, CRACM, CysLTR2, and Kv1.5 can be seen in FIGS. 18A-18E.

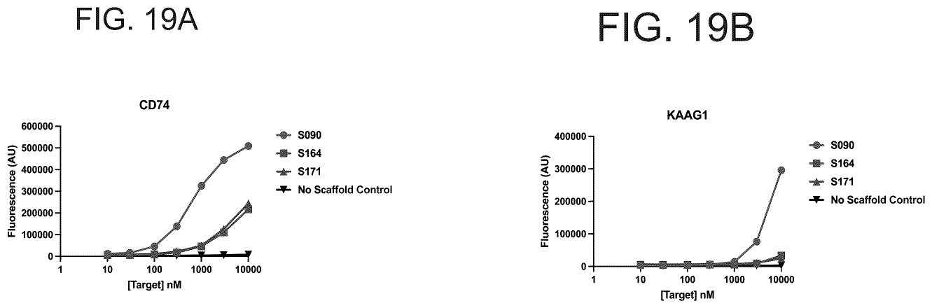

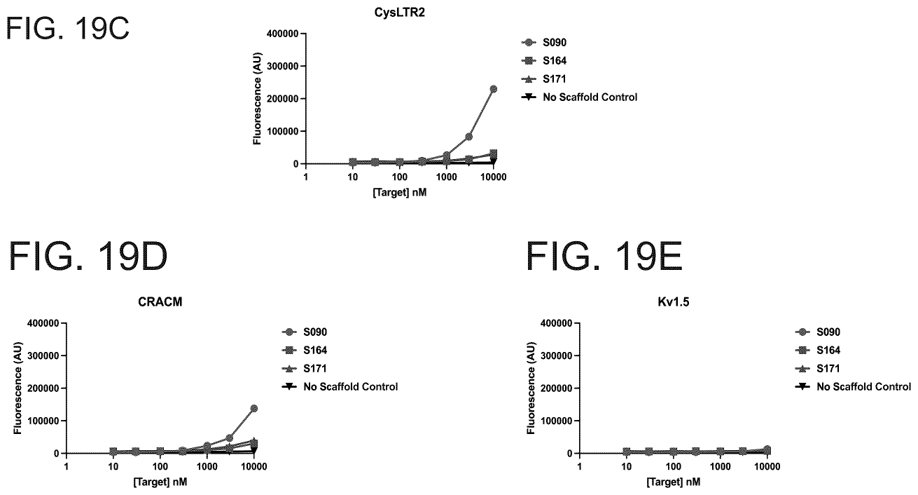

Engineered scaffolds S164 and S171 were computationally derived as described herein and the binding activities of these engineered scaffolds were measured in an ELISA assay as described herein relative to S090 and a negative control. Results for binding of CD74 (positive control), KAAG1, CRACM, CysLTR2, and Kv1.5 can be seen in FIG. 19A-19E.

TABLE 11 summarizes the results of the assays described herein and shows that the engineered scaffold proteins bind to target peptides with selectivity, and even if more than one target peptide can be bound, binding strength differs. FlhG is a non-specific hexamer protein, used as a positive control to demonstrate that it does not bind the target of interest.

TABLE 11
Targets vs Engineered Scaffolds

| | | | | | | | Urotensin | | |
|---|---|---|---|---|---|---|---|---|---|
| | CD74 | KAAG1 | Kv1.5 | CysLTR2 | CRACM | TRPV1 | IIR | TNFRSF13b | Kv1.6 |
| S005 | ++++ | + | − | ++ | ++ | ++ | − | − | − |
| S007 | ++++ | + | − | + | ++ | − | − | − | − |
| S090 | ++++ | +++ | − | +++ | + | ++ | + | − | − |
| S058 | ++++ | + | − | + | + | − | − | | |
| S065 | ++++ | − | − | − | + | − | − | − | − |
| S164 | ++++ | − | − | − | − | | | | |
| S171 | ++++ | − | − | − | − | | | | |
| FlhG | ++ | − | | | | | | | |
| S098 | ++++ | − | | ++ | + | − | − | − | − |
| S177 | | | | ++ | | | | | |

Key

| | | |
|---|---|---|
| − | <5000 | No significant binding |
| + | 50000 < x < 100000 | Weak binding |
| ++ | 100000 < x < 250000 | Moderate-weak binding |
| +++ | 250000 < x < 400000 | Moderate binding |
| ++++ | >400000 | Strong binding |

### Example 8. Selective Binding of Linear Epitopes

Next, ELISA assays as described in Example 7 were undertaken to determine linear epitopes of disordered regions of targets of interest. MERKFMSLQPSISVSEMEPNGTFSNNNSRNCTIENFKREFFP (SEQ ID NO: 926) is the N-terminal disordered region of CYSLTR2, a target of interest. Naturally occurring MHC molecules usually bind to peptides that range in size from 8-20 amino acids. However, certain linear epitopes may be greater than 20 amino acids in length, accordingly, narrowing the targeted linear epitope is beneficial to enhance binding activity. In the present assay, the ˜42 amino acids N-terminal disordered region was split into 3 N-terminally biotinylated peptides with a length of ˜21AA and an overlap of ˜10AA, MERKFMSLQPSISVSEMEPNG (SEQ ID NO: 927), SISVSEMEPNGTFSNNNSRNC (SEQ ID NO: 928), and TFSNNNSRNCTIENFKREFFP (SEQ ID NO: 929).

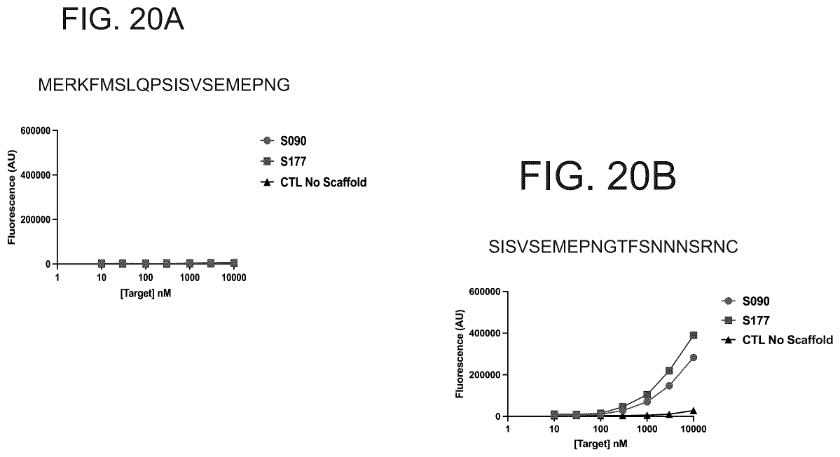

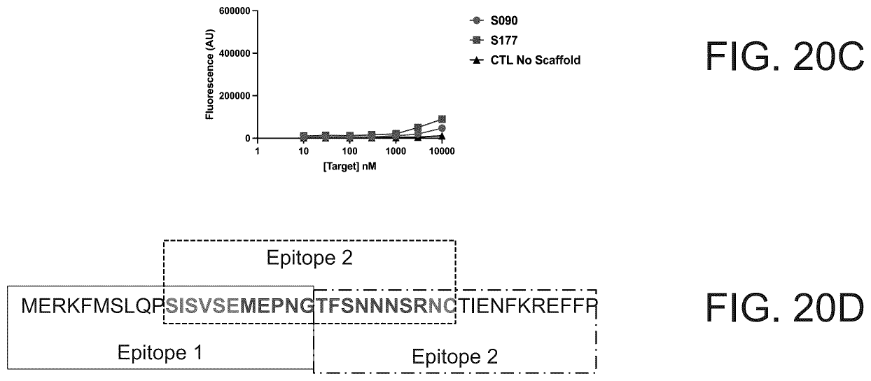

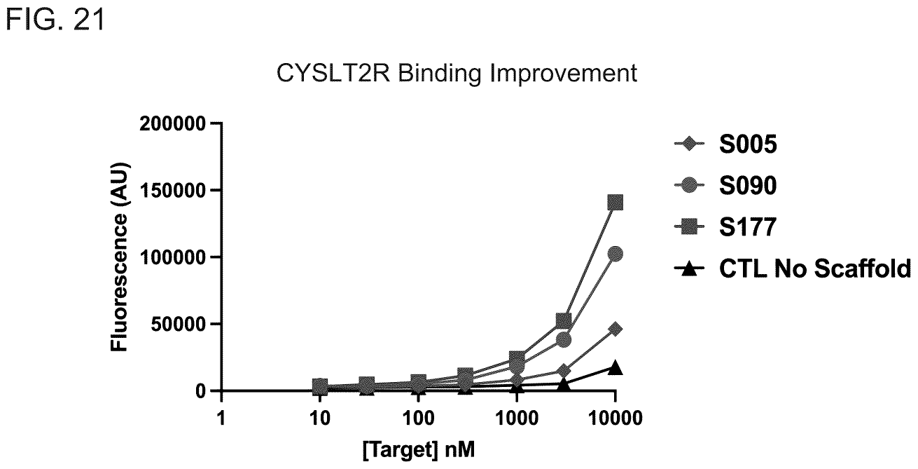

Results of S090 and S177 binding to the three separate linear epitopes relative to a negative control can be seen in FIGS. 20A-20D. Results indicate that targeting SISVSEMEPNGTFSNNNSRNC (SEQ ID NO: 928) results in the most increased amount of binding. S177 was computationally derived from S005 by methods described herein. Upon targeting the optimized linear epitope of CYSLT2R, SISVSEMEPNGTFSNNNSRNC (SEQ ID NO: 928), binding improvement can be seen with S005, S090 and S177 relative to a negative control in FIG. 21.

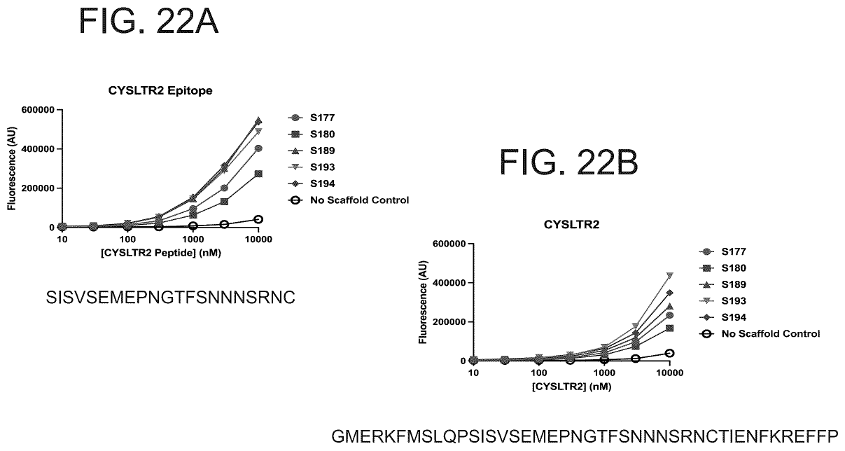

A similar assay was undertaken to compare binding of a short linear epitope (SISVSEMEPNGTFSNNNSRNC (SEQ ID NO: 928)) against a longer amino acid sequence of the disordered region of CYSLTR2 (GMERKFMSLQPSISVSEMEPNGTFSNNNSRNCTIENFKREFFP (SEQ ID NO: 930)). Assayed scaffolds include: S177 a computationally engineered variant, S189 which is derived from S177 with an extra dicysteine, S180 which is a wild-type, S193 which is a engineered scaffold protein linked to an N terminal low affinity class II-associated invariant chain peptide (CLIP peptide) with short linker, S194 which is an engineered scaffold protein with a low affinity CLIP peptide with linker in middle of the construct. Results can be seen compared to a negative control in FIGS. 22A-22B.

### Example 9: Engineered Scaffold Protein Binding Selectivity

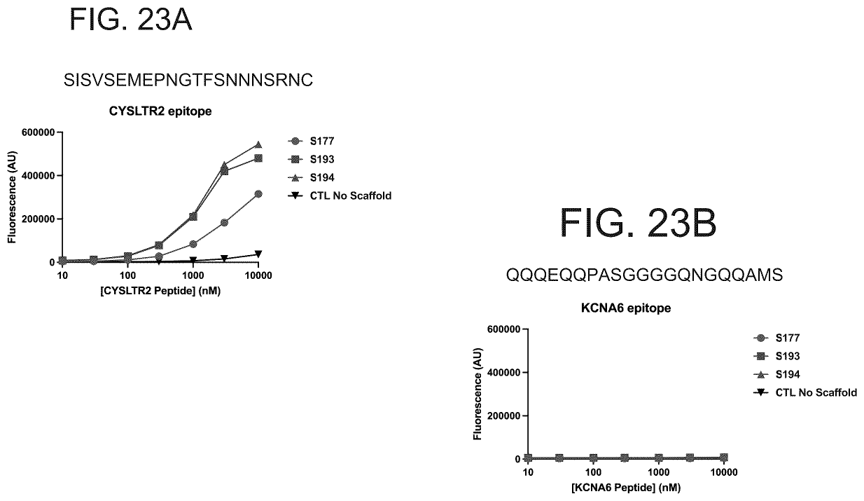

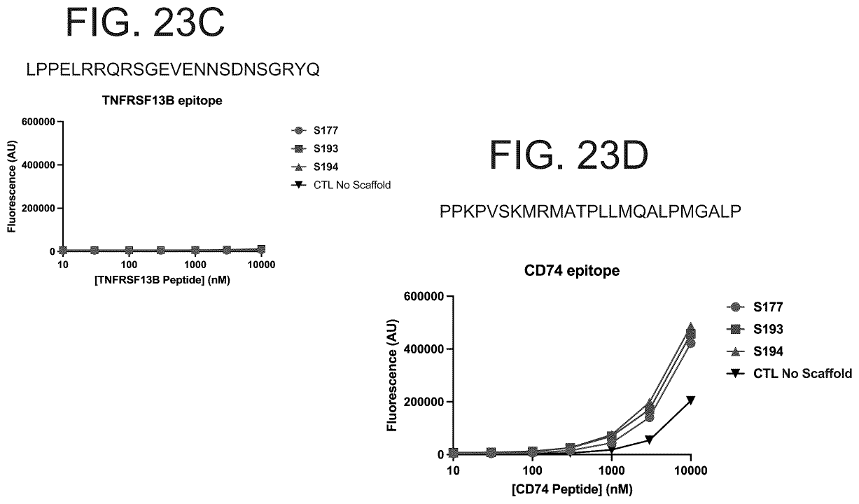

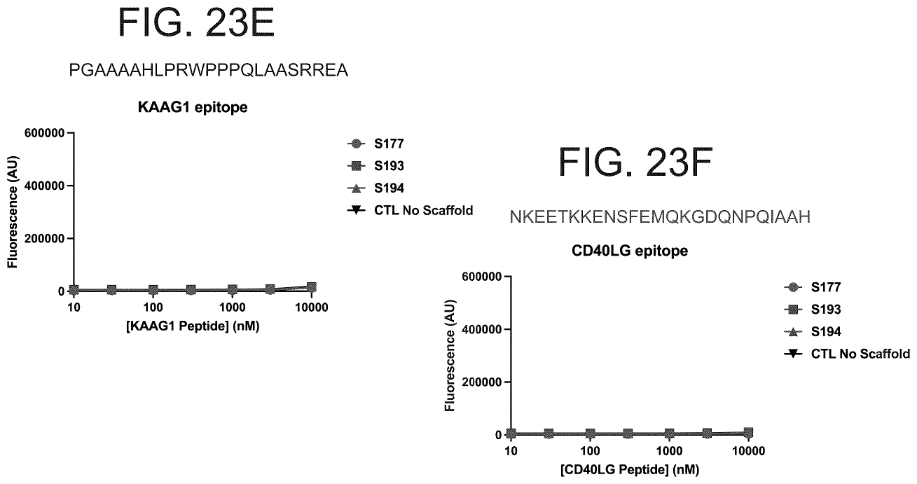

Next, binding selectivity of the short linear epitope (SISVSEMEPNGTFSNNNSRNC (SEQ ID NO: 928)) of CYSLTR2 was measured against the short linear epitopes for KCNA6, TNFRSF13B, KAAG1, CD40LG, and CD74 (positive control). Results can be seen in FIGS. 23A-23F. Selective binding was only found for CD74 (positive control) and CYSLTR2.

Likewise, TABLE 12 summarizes the results of the assays described herein, and represents a heat map of engineered scaffold proteins and binding selectivity to disordered regions of target peptides as measured by fluorescent signal strength. TABLE 12 shows that engineered scaffold proteins bind to target peptides with selectivity, and even if more than one target peptide can be bound, binding strength differs.

TABLE 12
Targets vs Scaffolds

| | | | | | | | Urotensin | | |
|---|---|---|---|---|---|---|---|---|---|
| | CD74 | KAAG1 | Kv1.5 | CysLTR2 | CRACM | TRPV1 | IIR | TNFRSF13b | Kv1.6 |
| S049 | +++ | − | | | | | | | |
| S055 | ++++ | + | | | | | | | |
| S056 | ++++ | + | | | | | | | |
| S068 | ++++ | − | | | | + | − | − | − |
| S077 | ++++ | − | | ++ | − | − | − | − | − |
| S078 | ++++ | − | | + | − | − | − | − | − |
| S084 | ++++ | − | | − | − | − | − | − | − |
| S134 | ++ | − | | | | | | | |
| S143 | ++++ | − | | | | | | | |

Key

| | | |
|---|---|---|
| − | <5000 | No significant binding |
| + | 50000 < x < 100000 | Weak binding |
| ++ | 100000 < x < 250000 | Moderate-weak binding |
| +++ | 250000 < x < 400000 | Moderate binding |
| ++++ | >400000 | Strong binding |

### Example 10: BLI Assay Using OCTET RED

Disordered epitopes of target proteins with a biotin were synthesized either chemically or expressed in E. coli and subsequently labeled with a biotin using a sortase mediated ligation of a short biotinylated peptide.

Streptavidin sensor tips for the BLI instrument were equilibrated in an equilibration buffer (1×PBS, 0.05% Tween 20, 0.5% BSA) for 5 minutes. Biotinylated polypeptide in the equilibration buffer at 0.5 μM was loaded on the streptavidin sensor over 60 s. After substrate loading, the sensor tips were washed in the equilibrating buffer for 5 minutes to block non-specific binding and to obtain a baseline reading. Peptide loaded and blocked tips were then introduced into wells containing polypeptides in the equilibrating buffer at varying concentrations. The binding and dissociation data are fit using the OCTET software to obtain kₒₙ, k_{off} and consequently KD, the binding affinity.

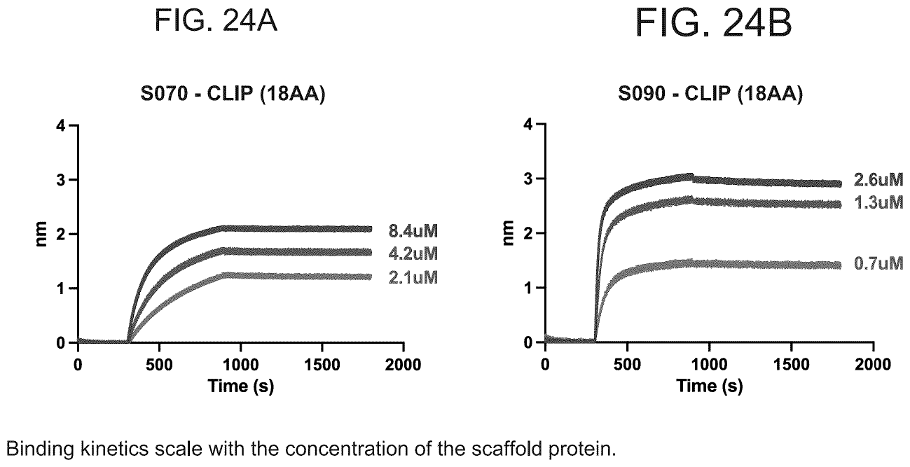

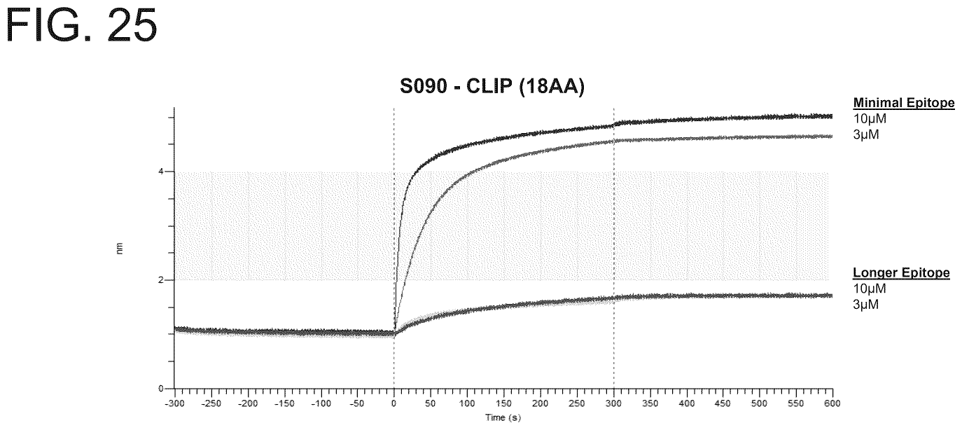

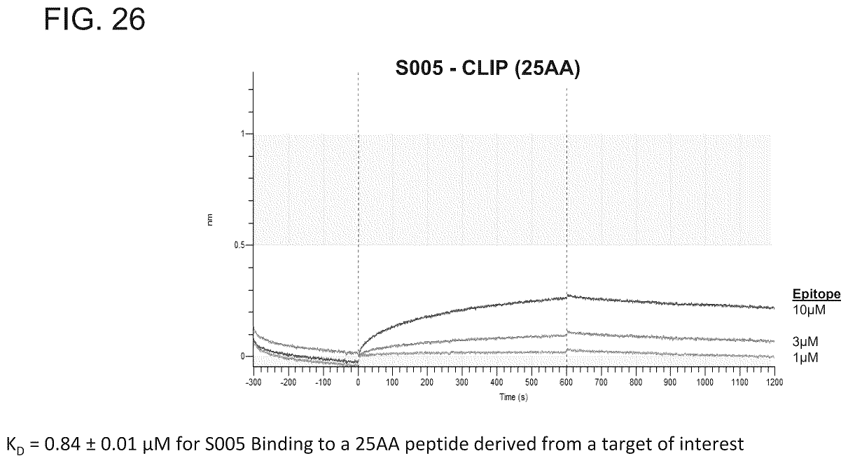

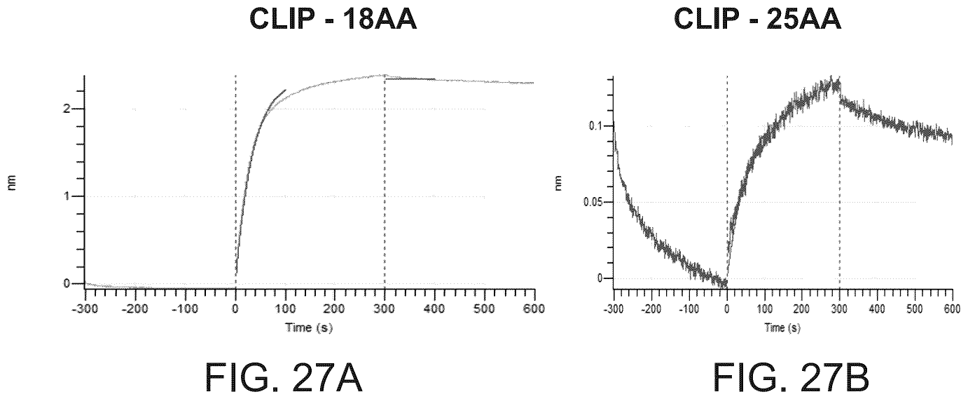

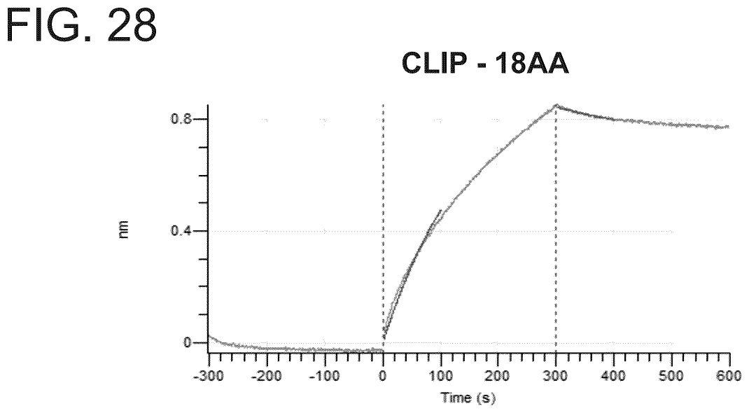

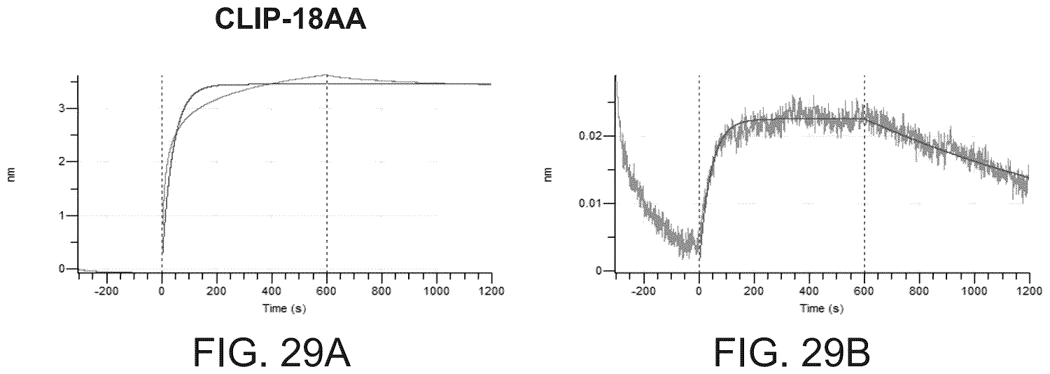

Results for S078 and S090 targeting an 18 amino acid linear epitope of CLIP can be seen in FIGS. 24A and 24B. FIGS. 25-29 demonstrates that engineered scaffold proteins, although engineered to target an 18 amino acid epitope of CLIP (CD74), can bind an 18 amino acid epitope and a 25 amino acid epitope. This effect is unexpected as native MHC II molecules are only known to bind to short peptides that are about 18-20 amino acids in length. Moreover, by comparing the binding of S090 vs. S005 to an 25AA long peptide sequence, binding by S090 (which is the iterative version of S005) to the target peptide is seen to be improved over binding by S005 (v1.1) demonstrating that optimizing the scaffold and rational design the scaffold has improved binding affinity (FIG. 25 and FIG. 26).

### Example 11: Binding of GPCRs

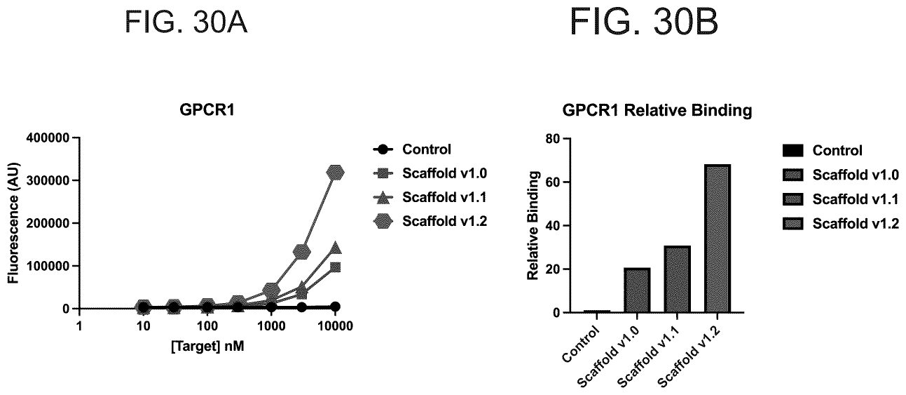

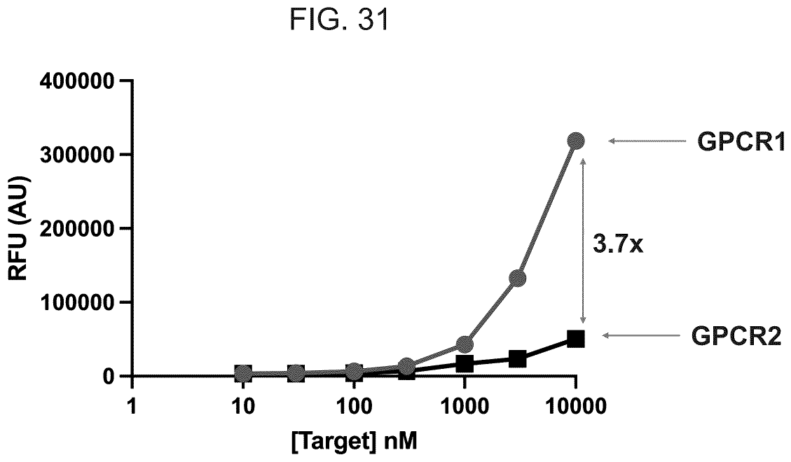

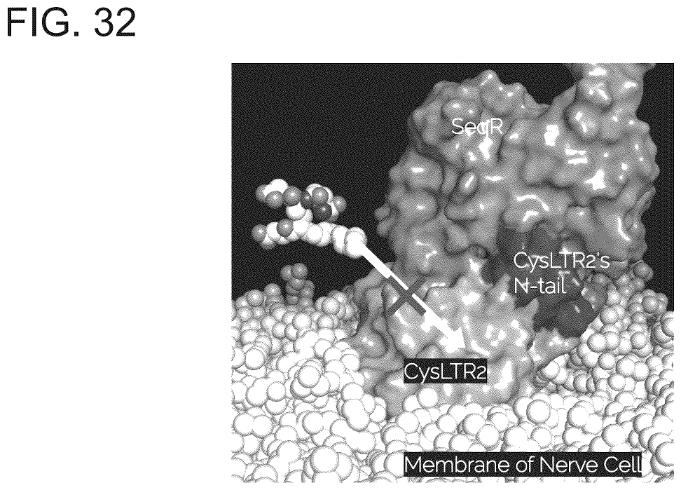

Engineered scaffold proteins were designed by rational engineering to enhance binding affinity to selected target peptides in the GPCR family. Engineered scaffolds included v1.0 (S007), v1.1 (S005), v1.2 (S090). The engineered scaffold proteins were generated as described in Example 5 and ELISA assays were run as described in Example 7. FIGS. 30A and 30B demonstrates that v1.2 exhibits the best performance and demonstrates that the engineered scaffold proteins bind the target peptide with >65-fold higher affinity compared to a non-specific protein binder. The best performing engineered scaffold was assayed to establish selectivity of the GPCR 1 target over a related GPCR2 target. Results in FIG. 31 demonstrates that v1.2 exhibits binding selectivity for the targeted GPCR over a related GPCR.

### Example 12: Engineered Scaffold Proteins Derived from Non-MHC Proteins

Caspases are proteases that bind to and cleave tetrapeptide sequences after an aspartate residue at the C-terminal end of the substrate sequence. For the proteolytic activity, a specific cysteine residue in the catalytic core is necessary. Caspase-derived engineered scaffold proteins are generated by mutating the cysteine in the enzyme active site to an alanine or other residue. By doing so, the proteolytic activity of caspases is abrogated. Moreover, residues in the binding unit of the caspase-derived engineered scaffold proteins are altered to increase specificity to particular sequences of target peptides and with a high binding affinity. This same approach can be employed to any class of proteins that bind to short polypeptide sequences from 4-50 amino acids, such as those described herein.

### Example 13: Engineered Scaffold Fusion Proteins

An E3 ligase is fused to an engineered scaffold protein at its C terminal end. The engineered scaffold protein is used to specifically bind to a disordered region on a target peptide with high affinity and selectivity. When the fusion protein binds to the target protein, the C-terminally fused E3 ligase is brought in close contact with the target peptide and can ubiquitinate the target peptide. The ubiquitinated protein is then sequestered and shuffled to a degradation pathway by natural cellular mechanisms, e.g., as described. This same approach can be used for modulating post translational modifications on the target peptide by fusing for example a kinase, protein phosphatase, etc.

Steps include producing the fusion protein of interest in soluble form, and separately, engineered scaffold protein and the fusion partner (e.g., E3 Ligase). Specificity of engineered scaffold protein-Ligase fusion to target peptide of interest is established using ELISA. Once the specificity is established, activity of the fusion protein is validated with a cell free ubiquitination assay. To establish activity inside cells, the fusion protein is encoded in a mammalian expression plasmid and transfected into mammalian cells (e.g., HEK293) and expression of the fusion protein is verified by Western blot analysis. Targeted degradation of the target peptide or protein is determined and validated by transfection and western blot analysis. The expected outcome is that in the presence of the fusion protein, the target protein is ubiquitinated whereas in the absence of the fusion protein, the target protein is not ubiquitinated.

From the foregoing description, it will be apparent that variations and modifications can be made to the invention described herein to adapt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1.-144. (canceled)

145. An engineered scaffold protein that can specifically bind to a disordered region in a target peptide, wherein the engineered scaffold protein comprises:
a. one or more binding domain comprising one or more binding units and one or more hinge units, wherein:
i. the one or more binding units is capable of binding to the disordered region of the target peptide and comprises one or more amino acid alterations relative to a wildtype (WT) counterpart, and
ii. the one or more hinge units is capable of stabilizing the structure of the binding domain for binding; and
b. one or more molecular transporters,
wherein binding activity of the target peptide by the engineered scaffold protein is increased relative to a WT counterpart of the engineered scaffold.

146. The engineered scaffold protein of claim 145, wherein the one or more binding units each comprises one or more helices comprising alpha helices, 3.10 helices, and/or pi helices.

147. The engineered scaffold protein of claim 145, wherein the binding unit comprises an amino acid sequence that is at least 65% identical to any one of the sequences in TABLE 4.

148. The engineered scaffold protein of claim 145, wherein the hinge unit comprises an amino acid sequence that is at least 65% identical to any one of the sequences in TABLE 5.

149. The engineered scaffold protein of claim 145, wherein the engineered scaffold protein comprises an amino acid sequence that is at least 65% identical to any one of the sequences set forth in TABLE 6.

150. The engineered scaffold protein of claim 145, wherein the binding unit comprises an elongated, clamshell, or binding groove architecture.

151. The engineered scaffold protein of claim 145, wherein the target peptide is comprised in a membrane protein, a secreted protein, or an extracellular protein.

152. The engineered scaffold protein of claim 145, wherein the disordered region is located in an internal loop, N-terminal tail, or C-terminal tail.

153. The engineered scaffold protein of claim 145, wherein the disordered region comprises a linear epitope of about 4 to about 40 amino acids.

154. The engineered scaffold protein of claim 145, wherein the one or more hinge units comprises a (a) concave (b) partially concave configuration, or a (c) flexible configuration that conforms to the disordered regions of the target peptide when the disordered region is bound by the engineered scaffold protein or any combination thereof.

155. The engineered scaffold protein of claim 145, wherein the one or more hinge units comprises one or more beta sheet strands, linear peptides, covalent interactions, non-covalent interactions, chemical agents, or any combination thereof.

156. The engineered scaffold protein of claim 145, wherein the engineered scaffold comprises
a) decreased immunogenicity relative to a corresponding WT;
b) increased solubility relative to a corresponding WT;
c) increased stability relative to a corresponding WT;
d) increased or decreased hydrophobicity relative to a corresponding WT;
e) increased or decreased hydrophilicity relative to a corresponding WT;
f) increased or decreased surface charge relative to a corresponding WT; or any combination of (a)-(f).

157. The engineered scaffold protein of claim 145, wherein upon being folded in its tertiary conformation, the binding unit comprises a three-dimensional conformation with one or more amino acids that are antisense to one or more amino acids of the disordered region.

158. The engineered scaffold protein of claim 145, wherein the molecular transporter is selected from the group consisting of cell penetrating peptides (CPPs), lipids, liposomes, lipid nanoparticles, steroids, polyamines, nanotubes, nanoparticles, dendrimers, peptoids, oligogcarbamates, HIV-binding peptides, HIV-1 Tat (HIV), Tat-derived peptides, Penetratin, VP22 derived or analog peptides, Pestivirus Erns, HSV, VP22 (Herpes simplex), MAP, KALA, PpT620, prolin-rich peptides, arginine-rich peptides, lysine-rich peptides, MPG-peptide(s), Pep-1, L-oligomers, Calcitonin peptide(s), Antennapedia-derived peptides, pAntp, pIsl, FGF, Lactoferrin, Transportan, Buforin-2, Bac715-24, SynB, SynB (1), pVEC, hCT-derived peptides, SAP, histones, modified or phosphorothioated single-stranded DNA (ssDNA), polymer-based materials, protein nanocarriers, transmembrane antibodies, nanocapsules (NCs), PEG coated hyaluronic acid nanoparticles (NPs), polymer NPs with glucose head groups, protein transduction domain mimics, anionic peptides, pyridylthiourea-modified polyethyleneimine (PEI), mutated lycotoxin, Sesbania mosaic virus like particles (VLPs), PEG- and arginine-capped lipids, microinjection and electroporation, and poly(lactide-co-glycolic acid) (PLGA) NPs.

159. The engineered scaffold protein of claim 145, wherein the engineered scaffold protein is configured as a multimer.

160. A fusion protein comprising the engineered scaffold protein of claim 145 and a fusion partner selected from an enzyme, a binding protein, a therapeutic agent, or a molecular tag.

161. The fusion protein of claim 160, wherein the fusion partner catalyzes post-translational modification, ubiquitination, proteolysis, or has chaperone activity.

162. The fusion protein of claim 160, wherein the fusion partner binds a region of the target protein distinct from the disordered region.

163. The fusion protein of claim 160, further comprising a half-life extension moiety selected from polyethylene glycol (PEG), human serum albumin (HSA), or a variant Fc region.

164. The engineered scaffold protein of claim 145, further comprising an epitope masking unit that is displaced upon binding the target peptide.

165. A method of binding a disordered region of a target protein, comprising contacting a target protein comprising said disordered region with the engineered scaffold protein of claim 145.

166. The method of claim 165, wherein binding by the engineered scaffold protein modulates the conformation or activity of the target protein.

167. The method of claim 165, wherein the target protein is a GPCR, receptor, ion channel, or secreted protein.

168. A method of treating or managing a disease or disorder in a subject in need thereof, comprising administering to the subject the engineered scaffold protein of claim 145.

169. A method of inducing an immune response in a subject in need thereof, comprising administering to the subject the engineered scaffold protein of claim 145.

170. A chimeric antigen receptor (CAR) T-cell comprising the engineered scaffold protein of claim 145 as a targeting domain.

171. The method of claim 168, wherein the one or more molecular transporter is a tissue-specific or cell-specific targeting moiety.

172. A method of detecting a target protein in a sample, comprising contacting the sample with the engineered scaffold protein of claim 145 labeled with a detectable moiety, and detecting the signal from the detectable moiety.

173. A method of screening binding partners comprising the use of the engineered scaffold protein of claim 145 to screen for screening protein-protein interactions or identifying binding partners.

174. A kit comprising the engineered scaffold protein of claim 145 for detecting a target protein.

175. A device comprising the engineered scaffold protein of claim 145 for detecting a compound in a sample.

176. An isolated nucleic acid encoding the engineered scaffold protein of claim 145.

177. An expression vector comprising the nucleic acid of claim 176.

178. A host cell comprising the expression vector of claim 177.

179. A polypeptide display library comprising a plurality of engineered scaffold proteins of claim 145, displayed on a virus, yeast, ribosome, or as an RNA-conjugated molecule, and a method of selecting scaffold proteins that bind to a disordered region of a target peptide.
